(19) United States
(12) Patent Application Publication
(10) Pub. No.: US 2026/0234709 A1
(43) Pub. Date: Aug. 13, 2026

(54) Methods of Making DNA Molecules and Compositions and Uses Thereof

(71) Applicant: ANJARIUM BIOSCIENCES AG, Schlieren (CH)

(72) Inventors: Joel De Beer, Schlieren (CH); Nicolas Meier, Schlieren (CH); Alexander Pekarsky, Schlieren (CH); Jorge Omar Yanez-Cuna, Schlieren (CH); Ivana Pastierikova, Schlieren (CH)

(21) Appl. No.: 19/156,797

(22) PCT Filed: Feb. 16, 2024
(86) PCT No.: PCT/EP2024/054075

§ 371 Date: Aug. 14, 2025

Related U.S. Application Data
(60) Provisional application No. 63/446,577, filed on Feb. 17, 2023.

(51) Int. Cl. C12Q 1/6853; A61K 48/00; C12N 15/864; C12N 15/867; C12Q 1/34; C12Q 1/48

(52) U.S. Cl. CPC C12Q 1/6853; A61K 48/005; A61K 48/0091; C12N 15/8645; C12N 15/867; C12Q 1/34; C12Q 1/48; C12Y 207/07; C12Y 301/00; C12N 2310/531; C12N 2740/15043; C12N 2740/15052; C12N 2750/14143; C12N 2750/14152

(57) ABSTRACT

The present disclosure relates to methods and kits for making hairpin-ended DNA molecules through amplification (e.g., isothermal amplification, e.g., rolling circle amplification (RCA), multiple displacement amplification (MDA)) of a circular DNA template, compositions comprising such made hairpin-ended DNA molecules, and uses thereof. Methods disclosed herein can produce transfection/transcription-ready high fidelity and high purity DNA molecules that are suitable for various uses (e.g., gene therapies).

This application claims the benefit of priority to U.S. Ser. No. 63/446,577, filed Feb. 17, 2023, which is incorporated herein by reference in its entirety.

This application contains a computer readable Sequence Listing which has been submitted in XML file format with this application, the entire content of which is incorporated by reference herein in its entirety. The Sequence Listing XML file submitted with this application is entitled “14497-014-228_SEQLISTING.xml”, was created on Feb. 15, 2024, and is 504,613 bytes in size.

### 1. FIELD

The present disclosure relates to methods and kits for making hairpin-ended DNA molecules through amplification (e.g., isothermal amplification, e.g., rolling circle amplification (RCA), multiple displacement amplification (MDA)) of circular DNA template, compositions comprising such made hairpin-ended DNA molecules, and uses thereof. Methods disclosed herein can produce transfection-ready and transcription-ready high fidelity and high purity DNA molecules that are suitable for various uses (e.g., gene therapies, in vitro transcription).

### 2. BACKGROUND

Gene therapy aims to introduce genes into target cells to treat or prevent disease. By supplying a transcription cassette with an active gene product (e.g., transgene), gene therapy can improve clinical outcomes, such as a gain of positive function effect and a loss of negative function effect. Other improved clinical outcomes include anti-tumor effects. Delivery and expression of a corrective gene in target cells of patients can achieved by non-viral delivery (e.g., liposomal) or viral delivery methods (e.g., engineered viruses and viral gene delivery vectors). Of the known viral vectors (e.g., recombinant retrovirus, recombinant lentivirus, recombinant adenovirus, and the like), AAV systems are gaining popularity as versatile vectors in gene therapy.

However, viral vectors have several deficiencies as gene delivery vectors. First, packaging the transcription cassette into the viral vectors depends on viral life cycle and viral proteins. Such dependency limits the size of transgenes (e.g., less than 150,000 Da protein coding capacity for AAV) that can be delivered by the viral vectors and requires the presence of specific viral sequences to ensure efficient replication and packaging (e.g., Rep-Binding Element), which can destabilize the expression cassette. Thus, more than one viral particle may be required to deliver large transgenes (e.g., transgenes encoding proteins larger than 150,000 Da, or transgenes longer than about 4.7 Kb). However, use of two or more viral constructs can increase the risk of re-activation of the viral genome. Furthermore, the use of a viral Rep or Nonstructural Protein 1 Binding Element may increase the risk of vector mobilization in patients.

Moreover, viral particles used for gene therapy are often derived from wild-type viruses to which a subset of human population has been exposed during their lifetime. These patients carry neutralizing antibodies which can hinder gene therapy efficacy as further described in Snyder, Richard O., and Philippe Moullier. Adeno-associated virus: methods and protocols. Totowa, NJ: Humana Press, 2011. For seronegative patients, the capsids of viral vectors are often immunogenic, preventing the re-administration of the viral vector therapy to patients should an initial dose not be sufficient or the therapy wears off.

Isothermal amplification, such as rolling circle amplification (RCA) and multiple displacement amplification (MDA), has been used for detecting the presence of circular DNA (e.g., viral DNA). However, it is challenging to use isothermal amplification in generating therapeutic DNA molecules because of the high viscosity and low fidelity of the hyper-branched DNA structures generated by isothermal amplification such as MDA. The high viscosity of the DNA products generated by MDA renders it difficult to digest or process these products for further subsequence application.

As such, there is unmet need for making transfection-ready and transfection-ready DNA molecules using isothermal amplification. There is also unmet need for making non-viral vehicles to deliver transgenes for gene therapies.

### 3. SUMMARY

In one aspect, the present disclosure provides a method for preparing hairpin-ended DNA molecules, wherein the method comprises:
a. providing a circular DNA molecule as a template;
b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
   i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
   ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
   iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
   iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand;
c. incubating the amplification product with one or more nicking endonucleases recognizing the four restriction sites;
d. denaturing and thereby creating a DNA fragment that comprises the two single strand DNA overhangs as specified in step b upon separation of the top from the bottom strand; and
e. annealing the single strand DNA overhangs and thereby creating a hairpinned inverted repeat on each end of the DNA fragment resulting from the denaturing step to produce the hairpin-ended DNA molecule.

In another aspect, the present disclosure provides a method for amplifying precursors of hairpin-ended DNA molecules, wherein the method comprises:
a. providing a circular DNA molecule as a template; and
b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
   i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
   ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
   iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
   iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand.

In certain embodiments, the method further comprises: c. incubating the amplification product with one or more nicking endonucleases recognizing the four restriction sites; d. denaturing and thereby creating a DNA fragment that comprises the two single strand DNA overhangs as specified in step b; and e. annealing the single strand DNA overhangs and thereby creating a hairpinned inverted repeat on each end of the DNA fragment resulting from the denaturing step to produce the hairpin-ended DNA molecule.

In another aspect, the present disclosure provides a method for preparing precursors of hairpin-ended DNA molecules, wherein the method comprises:
a. providing a circular DNA molecule as a template;
b. incubating the template with a polymerase and a primer pair under conditions suitable for amplification to produce at least one amplification product and suitable for at least 2-fold amplification of the template, wherein the amplification product comprises
   i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
   ii. a restriction enzyme site wherein the restriction enzyme site is located outside a segment comprising the first inverted repeat at one end, the sequence of interest, and the second inverted repeat at the other end; and
c. incubating the amplification product with a restriction enzyme that cleaves the restriction enzyme site to produce the precursor of the hairpin-ended DNA molecule comprising the first inverted repeat, the sequence of interest, and the second inverted repeat.

In certain embodiments, the template comprises no more than one type of restriction enzyme site, wherein the restriction enzyme site is present 1, 2, 3, 4, 5, 6, 7, 8, 9, 10, or more times in the template.

In another aspect, the present disclosure provides a method for preparing precursors of a hairpin-ended DNA molecules, wherein the method comprises:
a. providing a circular DNA molecule as a template;
b. incubating the template with a polymerase and a primer pair under conditions suitable for amplification to produce at least one amplification product and suitable for at least 2-fold amplification of the template, wherein the amplification product comprises
   i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
   ii. a fifth and a sixth restriction sites for nicking endonuclease arranged on opposite strands, wherein the fifth and sixth restriction sites are located outside a segment comprising the first inverted repeat at one end, the sequence of interest, and the second inverted repeat at the other end; and
c. incubating the amplification product with a nicking endonuclease that nicks the fifth and sixth restriction sites to produce the precursor of the hairpin-ended DNA molecule comprising the first inverted repeat, the sequence of interest, and the second inverted repeat.

In certain embodiments, the template comprises no additional restriction sites for nicking endonuclease, optionally wherein each of the fifth and sixth restriction sites for nicking endonuclease is present 1, 2, 3, 4, 5, 6, 7, 8, 9, 10 or more times in the template.

In certain embodiments, (i) nicks created by nicking at the fifth and sixth restriction sites are 1, 2, 3, 4, 5, 6, 7, 8, 9, 10, 11, 12, 13, 14, 15, 16, 17, 18, 19, or 20 nucleotides apart; and (ii) single strand DNA overhangs created by nicking at the fifth and sixth restriction sites do not anneal at detectable levels inter- or intramolecularly under conditions that favor annealing of the first and/or second inverted repeat.

In certain embodiments, the method further comprises: d. incubating the precursor of the hairpin-ended DNA molecule with one or more nicking endonucleases recognizing the first, second, third, and fourth restriction site; e. denaturing and thereby creating a DNA fragment that comprises the two single strand DNA overhangs as specified in step b upon separation of the top from the bottom strand; and f. annealing the single strand DNA overhangs of the DNA fragment and thereby creating a hairpinned inverted repeat on each end of the DNA fragment resulting from the denaturing step to produce the hairpin-ended DNA molecule.

In certain embodiments, the method produces non-hairpin-ended DNA molecules comprising at least one non-hairpin end, and the method further comprises digesting the non-hairpin-ended DNA molecules with an exonuclease, wherein the hairpin-ended DNA molecule is resistant to digestion by the exonuclease.

In certain embodiments, the amplification product comprises an additional restriction enzyme site and/or additional restriction sites for nicking endonuclease located outside a segment comprising the first inverted repeat at one end, the sequence of interest, and the second inverted repeat at the other end, and the method further comprises creating additional non-hairpin-ended DNA molecules by cleaving the additional restriction enzyme site and/or nicking the additional restriction sites for nicking endonuclease.

In another aspect, the present disclosure provides a method for preparing a composition comprising pure hairpin-ended DNA molecules, wherein the method comprises:
a. providing a circular DNA molecule as a template;
b. incubating the template with a polymerase and a primer pair under conditions suitable for amplification to produce at least one amplification product and suitable for at least 2-fold amplification of the template, wherein the amplification product comprises
   i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
   ii. a restriction enzyme site wherein the restriction enzyme site is located outside a segment comprising the first inverted repeat at one end, the sequence of interest, and the second inverted repeat at the other end;
c. incubating the amplification product with one or more nicking endonucleases recognizing the four restriction sites;
d. denaturing and thereby creating a DNA fragment that comprises the two single strand DNA overhangs as specified in step b upon separation of the top from the bottom strand;
e. annealing the single strand DNA overhangs and thereby creating a hairpinned inverted repeat on each end of the DNA fragment resulting from the denaturing step to produce a hairpin-ended DNA molecule comprising the sequence of interest and a hairpin-ended DNA molecule comprising the restriction enzyme site;
f. incubating the hairpin-ended DNA molecule comprising the restriction enzyme site with a restriction enzyme that cleaves at the restriction enzyme site to produce a non-hairpin-ended DNA molecule comprising at least one non-hairpin end; and
g. digesting the non-hairpin-ended DNA molecules with an exonuclease, wherein the hairpin-ended DNA molecule comprising the sequence of interest is resistant to digestion by the exonuclease.

In certain embodiments, the template comprises no more than one of the restriction enzyme site.

In another aspect, the present disclosure provides a method for preparing a composition comprising pure hairpin-ended DNA molecules, wherein the method comprises:
a. providing a circular DNA molecule as a template;
b. incubating the template with a polymerase and a primer pair under conditions suitable for amplification to produce at least one amplification product and suitable for at least 2-fold amplification of the template, wherein the amplification product comprises
   i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
   ii. a fifth and a sixth restriction sites for nicking endonuclease arranged on opposite strands, wherein the fifth and sixth restriction sites are located outside a segment comprising the first inverted repeat at one end, the sequence of interest, and the second inverted repeat at the other end; and
c. incubating the amplification product with one or more nicking endonucleases recognizing the four restriction sites;
d. denaturing and thereby creating a DNA fragment that comprises the two single strand DNA overhangs as specified in step b upon separation of the top from the bottom strand;
e. annealing the single strand DNA overhangs and thereby creating a hairpinned inverted repeat on each end of the DNA fragment resulting from the denaturing step to produce a hairpin-ended DNA molecule comprising the sequence of interest and a hairpin-ended DNA molecule comprising the fifth and sixth restriction site;
f. incubating the hairpin-ended DNA molecule comprising the fifth and sixth restriction sites with a nicking endonuclease that nicks the fifth and sixth restriction sites to produce a non-hairpin-ended DNA molecule comprising at least one non-hairpin end; and
g. digesting the non-hairpin-ended DNA molecules with an exonuclease, wherein the hairpin-ended DNA molecule comprising the sequence of interest is resistant to digestion by the exonuclease.

In certain embodiments, (i) the nicks created by nicking at the fifth and sixth restriction sites are 1, 2, 3, 4, 5, 6, 7, 8, 9, 10, 11, 12, 13, 14, 15, 16, 17, 18, 19, or 20 nucleotides apart; and (ii) the single strand DNA overhangs created by nicking at the fifth and sixth restriction sites do not anneal at detectable levels inter- or intramolecularly under conditions that favor annealing of the first and/or second inverted repeat.

In certain embodiments, the amplification product comprises an additional restriction enzyme site and/or additional restriction sites for nicking endonuclease located outside a segment comprising the first inverted repeat at one end, the sequence of interest, and the second inverted repeat at the other end, and the method further comprises creating additional non-hairpin-ended DNA molecules by cleaving the additional restriction enzyme site and/or nicking the additional restriction sites for nicking endonuclease.

In another aspect, the present disclosure provides a method for amplifying precursors of hairpin-ended DNA molecules, wherein the method comprises:
a. providing a circular DNA molecule as a template comprising a methylated methylation-sensitive restriction enzyme (MSRE)-recognition site; and
b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
   i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
   ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
   iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
   iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
c. incubating the template with an MSRE, wherein the amplification product comprises an unmethylated MSRE-recognition site located outside a segment comprising the first inverted repeat at one end, the sequence of interest, and the second inverted repeat at the other end, and the MSRE cleaves the amplification product at the unmethylated MSRE-recognition site.

In certain embodiments, the circular DNA molecule is incubated with the polymerase and the MSRE concurrently, or the circular DNA molecule is incubated with the polymerase prior to the MSRE.

In certain embodiments, the method further comprises: d. incubating the MSRE-cleaved amplification products with one or more nicking endonucleases recognizing the four restriction sites, thereby creating the two single strand DNA overhangs as specified in step b upon separation of the top from the bottom strand; e. denaturing and thereby creating a DNA fragment that comprises the two single strand DNA overhangs; and f. annealing the single strand DNA overhangs and thereby creating a hairpinned inverted repeat on each end of the DNA fragment resulting from the denaturing step to produce a hairpin-ended DNA molecule.

In another aspect, the present disclosure provides a method for preparing hairpin-ended DNA molecules, wherein the method comprises:
a. providing a circular DNA molecule comprising a methylated methylation-sensitive nicking endonuclease (MSNE)-restriction site;
b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
   i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
   ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
   iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
   iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
c. incubating the template with an MSNE, wherein the amplification product comprises two unmethylated MSNE-recognition sites located outside a segment comprising the first inverted repeat at one end, the sequence of interest, and the second inverted repeat at the other end, and the MSNE cleaves the amplification product at the two unmethylated MSNE-recognition sites.

In certain embodiments, the circular DNA molecule is incubated with the polymerase and the MSNE concurrently, or the circular DNA molecule is incubated with the polymerase prior to the MSNE.

In certain embodiments, the method further comprises: d. incubating the MSNE-cleaved amplification products with one or more nicking endonucleases recognizing the four restriction sites, thereby creating the two single strand DNA overhangs as specified in step b upon separation of the top from the bottom strand; e. denaturing and thereby creating a DNA fragment that comprises the two single strand DNA overhangs; and f. annealing the single strand DNA overhangs and thereby creating a hairpinned inverted repeat on each end of the DNA fragment resulting from the denaturing step to produce a hairpin-ended DNA molecule.

In another aspect, the present disclosure provides a method for preparing hairpin-ended DNA molecules, wherein the method comprises:
a. providing a circular DNA molecule as a template comprising a methylated MSRE-recognition site; and
b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
   i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
   ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
   iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
   iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand;
c. incubating the template with an MSRE, wherein the amplification product comprises an unmethylated MSRE-recognition site located outside the segment comprising the first inverted repeat at one end, the sequence of interest, and the second inverted repeat at the other end, and the MSRE cleaves the amplification product at the unmethylated MSRE-recognition site;
d. incubating the MSRE-cleaved amplification products with one or more nicking endonucleases recognizing the four restriction sites, thereby creating the two single strand DNA overhangs as specified in step b upon separation of the top from the bottom strand;
e. denaturing and thereby creating a DNA fragment that comprises the two single strand DNA overhangs; and
f. annealing the single strand DNA overhangs and thereby creating a hairpinned inverted repeat on each end of the DNA fragment resulting from the denaturing step to produce a hairpin-ended DNA molecule.

In certain embodiments, the method produces non-hairpin-ended DNA molecules comprising at least one non-hairpin end, and the method further comprises digesting the non-hairpin-ended DNA molecules with an exonuclease, wherein the hairpin-ended DNA molecule is resistant to digestion by the exonuclease.

In another aspect, the present disclosure provides a method for preparing hairpin-ended DNA molecules, wherein the method comprises:
a. providing a circular DNA molecule comprising a methylated MSNE-restriction site;
b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
   i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
   ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
   iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
   iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
c. incubating the template with an MSNE, wherein the amplification product comprises two unmethylated MSNE-recognition sites located outside the segment comprising the first inverted repeat at one end, the sequence of interest, and the second inverted repeat at the other end, and the MSNE cleaves the amplification product at the two unmethylated MSNE-recognition sites;
d. incubating the MSNE-cleaved amplification products with one or more nicking endonucleases recognizing the four restriction sites, thereby creating the two single strand DNA overhangs as specified in step b upon separation of the top from the bottom strand;
e. denaturing and thereby creating a DNA fragment that comprises the two single strand DNA overhangs; and
f. annealing the single strand DNA overhangs and thereby creating a hairpinned inverted repeat on each end of the DNA fragment resulting from the denaturing step to produce a hairpin-ended DNA molecule.

In certain embodiments, (i) the nicks created by nicking at the two unmethylated restriction sites are 1, 2, 3, 4, 5, 6, 7, 8, 9, 10, 11, 12, 13, 14, 15, 16, 17, 18, 19, or 20 nucleotides apart; and (ii) the single strand DNA overhangs created by nicking at the two unmethylated restriction sites do not anneal at detectable levels inter- or intramolecularly under conditions that favor annealing of the first and/or second inverted repeat.

In certain embodiments, the method further produces non-hairpin-ended DNA molecules comprising at least one non-hairpin end, and the method further comprises digesting the non-hairpin-ended DNA molecules with one or more exonucleases, wherein the hairpin-ended DNA molecule is resistant to digestion by the one or more exonucleases.

In certain embodiments, the method further comprises exchanging a buffer, concentrating the amplification product, and/or removing the circular DNA molecule, polymerase, and/or primer pair, after completion of step b and before initiation of step c.

In certain embodiments, the method further comprises exchanging a buffer, concentrating the amplification product, and/or removing the circular DNA molecule, polymerase, and/or primer pair, after completion of step c and before initiation of step d.

In certain embodiments, the sequence of interest comprises a transcription unit encoding a therapeutic protein. In certain embodiments, the sequence of interest comprises a transcription unit encoding an RNA for in vitro transcription (IVT). In certain embodiments, the sequence of interest comprises a gene promoter, an AAV ITR, or a synthetic DNA template to be integrated into a genome. In certain embodiments, the circular DNA molecule is a single-stranded circular DNA molecule or a double stranded circular DNA molecule. In certain embodiments, the amplification is an isothermal amplification.

In certain embodiments, the isothermal amplification is rolling circle amplification (RCA) and/or multiple displacement amplification (MDA).

In another aspect, the present disclosure provides a kit for preparing hairpin-ended DNA molecules, comprising:
a. a circular DNA molecule as a template, wherein an amplification product amplified from the template comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
   i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
   ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
   iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
   iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand;
b. a DNA polymerase suitable for amplification;
c. a primer pair; and
d. one or more nicking endonucleases recognizing the four restriction sites in the amplification product.

In another aspect, the present disclosure provides a kit for amplifying precursors of hairpin-ended DNA molecules, comprising:
a. a circular DNA molecule as a template, wherein an amplification product amplified from the template comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
   i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
   ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
   iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
   iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand;
b. a DNA polymerase suitable for amplification; and
c. a primer pair.

In certain embodiments, the kit further comprises one or more nicking endonucleases recognizing the four restriction sites in the amplification product.

In another aspect, the present disclosure provides a kit for preparing precursors of hairpin-ended DNA molecules, comprising:
a. a circular DNA molecule as a template, wherein an amplification product amplified from the template comprises:
   i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
   ii. a restriction enzyme site wherein the restriction enzyme site is located outside a segment comprising the first inverted repeat at one end, the sequence of interest, and the second inverted repeat at the other end;
b. a DNA polymerase suitable for amplification;
c. a primer pair; and
d. a restriction enzyme that recognizes the restriction enzyme site.

In another aspect, the present disclosure provides a kit for preparing precursors of hairpin-ended DNA molecules, comprising:
a. a circular DNA molecule as a template, wherein an amplification product amplified from the template comprises:
   i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
   ii. a fifth and a sixth restriction sites for nicking endonuclease arranged on opposite strands, wherein the fifth and sixth restriction sites are located outside a segment comprising the first inverted repeat at one end, the sequence of interest, and the second inverted repeat at the other end;
b. a DNA polymerase suitable for amplification;
c. a primer pair; and
d. a nicking endonuclease that recognizes the fifth and a sixth restriction site.

In certain embodiments, the kit further comprises one or more nicking endonucleases recognizing the first, second, third, and forth restriction sites in the amplification product.

In another aspect, the present disclosure provides a kit for preparing a composition comprising pure hairpin-ended DNA molecules, comprising:
a. a circular DNA molecule as a template, wherein an amplification product amplified from the template comprises:
   i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
   ii. a restriction enzyme site wherein the restriction enzyme site is located outside a segment comprising the first inverted repeat at one end, the sequence of interest, and the second inverted repeat at the other end;
b. a DNA polymerase suitable for amplification;
c. a primer pair;
d. a restriction enzyme that recognizes the restriction enzyme site;
e. one or more nicking endonucleases that recognizes the first, second, third, and forth restriction sites in the amplification product; and
f. an exonuclease.

In another aspect, the present disclosure provides a kit for preparing a composition comprising pure hairpin-ended DNA molecules, comprising:
a. a circular DNA molecule as a template, wherein an amplification product amplified from the template comprises:
   i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
   ii. a fifth and a sixth restriction sites for nicking endonuclease arranged on opposite strands, wherein the fifth and sixth restriction sites are located outside a segment comprising the first inverted repeat at one end, the sequence of interest, and the second inverted repeat at the other end;
b. a DNA polymerase suitable for amplification;
c. a primer pair;
d. a nicking endonuclease that recognizes the fifth and a sixth restriction site;
e. one or more nicking endonucleases that recognizes the first, second, third, and forth restriction sites in the amplification product; and
f. an exonuclease.

In another aspect, the present disclosure provides a kit for amplifying precursors of hairpin-ended DNA molecules, comprising:
a. a circular DNA molecule as a template comprising a methylated MSRE-recognition site, wherein an amplification product amplified from the template comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
   i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
   ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
   iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
   iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
b. an MSRE that recognizes and cleaves the amplification product at an unmethylated MSRE-recognition site located outside a segment comprising the first inverted repeat at one end, the sequence of interest, and the second inverted repeat at the other end;
c. a DNA polymerase suitable for amplification; and
d. a primer pair.

In another aspect, the present disclosure provides a kit for preparing hairpin-ended DNA molecules, comprising:
a. a circular DNA molecule comprising a methylated MSNE-restriction site, wherein an amplification product amplified from the template comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
   i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
   ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
   iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
   iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
b. an MSNE that recognizes and nicks the amplification product at the two unmethylated MSNE-recognition sites located outside a segment comprising the first inverted repeat at one end, the sequence of interest, and the second inverted repeat at the other end;
c. a DNA polymerase suitable for amplification; and
d. a primer pair.

In certain embodiments, the kit further comprises one or more nicking endonucleases that recognize the first, second, third, and forth restriction sites in the amplification product.

In certain embodiments, the kit further comprises an exonuclease.

In certain embodiments, the amplification is an isothermal amplification. In certain embodiments, the isothermal amplification is rolling circle amplification (RCA) and/or multiple displacement amplification (MDA).

Also provided herein is a method of producing AAV vectors for use in gene therapy comprising: (a) transfecting a host cell with at least one hairpin-ended DNA molecule for production of AAV particles, wherein the hairpin-ended DNA molecule has been produced according to a method and/or using a kit described herein; and (b) harvesting the AAV particles. In certain embodiments, step a comprises co-transfecting the host cell with (i) a hairpin-ended DNA molecule encoding an AAV vector genome and (ii) one or more DNA molecules encoding Rep protein(s), AAV capsid protein(s), and/or helper plasmid(s). In certain embodiments, step a comprises co-transfecting the host cell with (i) a hairpin-ended DNA molecule encoding an AAV vector genome; (ii) a hairpin-ended DNA molecule encoding Rep proteins and AAV capsid proteins; and (iii) a hairpin-ended DNA molecule encoding helper plasmids.

Also provided herein is a method of producing lentiviral vectors for use in gene therapy comprising: (a) transfecting a host cell with at least one hairpin-ended DNA molecule for production of lentiviral particles, wherein the hairpin-ended DNA molecule has been produced according to a method and/or using a kit provided herein; and (b) harvesting the lentiviral particles. In certain embodiments, step a comprises co-transfecting the host cell with (i) a hairpin-ended DNA molecule encoding a lentiviral transfer vector and (ii) one or more DNA molecules encoding packaging and/or envelope proteins selected from the group consisting of VSV-G protein(s)), Tat proteins, Rev protein(s), Gag protein(s), and Pol protein(s). In certain embodiments, step a comprises co-transfecting the host cell with (i) a hairpin-ended DNA molecule encoding a lentiviral transfer vector; (ii) a hairpin-ended DNA molecule encoding Rev protein; (iii) a hairpin-ended DNA molecule encoding Gag and Pol proteins; and (iv) a hairpin-ended DNA molecule encoding VSV-G protein.

Also provided herein is a method of producing RNA comprising: (a) transcribing a hairpin-ended DNA molecule, or a fragment thereof, for production of RNA, wherein the hairpin-ended DNA molecule comprises a transcription unit suitable for in vitro transcription (IVT) and has been produced according to a method described herein; and (b) harvesting the RNA product. In certain embodiments, the transcribing comprises the contacting the hairpin-ended DNA molecule, or fragment thereof, with an in vitro transcription reaction system comprising an RNA polymerase and ribonucleotides.

### 3.1 Illustrative Embodiments

Illustrative Embodiments of the present disclosure are provided in the paragraphs below:
1. A method for preparing hairpin-ended DNA molecules, wherein the method comprises:
   a. providing a circular DNA molecule as a template;
   b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand;
   c. incubating the amplification product with one or more nicking endonucleases recognizing the four restriction sites;
   d. denaturing and thereby creating a DNA fragment that comprises the two single strand DNA overhangs as specified in step b upon separation of the top from the bottom strand; and
   e. annealing the single strand DNA overhangs and thereby creating a hairpinned inverted repeat on each end of the DNA fragment resulting from the denaturing step to produce the hairpin-ended DNA molecule.
2. A method for amplifying precursors of hairpin-ended DNA molecules, wherein the method comprises:
   a. providing a circular DNA molecule as a template; and
   b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand.
3. The method of paragraph 2, further comprising
   c. incubating the amplification product with one or more nicking endonucleases recognizing the four restriction sites;
   d. denaturing and thereby creating a DNA fragment that comprises the two single strand DNA overhangs as specified in step b; and
   e. annealing the single strand DNA overhangs and thereby creating a hairpinned inverted repeat on each end of the DNA fragment resulting from the denaturing step to produce the hairpin-ended DNA molecule.
4. A method for preparing precursors of hairpin-ended DNA molecules, wherein the method comprises:
   a. providing a circular DNA molecule as a template;
   b. incubating the template with a polymerase and a primer pair under conditions suitable for amplification to produce at least one amplification product and suitable for at least 2-fold amplification of the template, wherein the amplification product comprises
      i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
         (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
         (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
         (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
         (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
      ii. a restriction enzyme site wherein the restriction enzyme site is located outside a segment comprising the first inverted repeat at one end, the sequence of interest, and the second inverted repeat at the other end; and
   c. incubating the amplification product with a restriction enzyme that cleaves the restriction enzyme site to produce the precursor of the hairpin-ended DNA molecule comprising the first inverted repeat, the sequence of interest, and the second inverted repeat.
5. The method of paragraph 4, wherein the template comprises no more than one type of restriction enzyme site, wherein the restriction enzyme site is present 1, 2, 3, 4, 5, 6, 7, 8, 9, 10, or more times in the template.
6. A method for preparing precursors of a hairpin-ended DNA molecules, wherein the method comprises:
   a. providing a circular DNA molecule as a template;
   b. incubating the template with a polymerase and a primer pair under conditions suitable for amplification to produce at least one amplification product and suitable for at least 2-fold amplification of the template, wherein the amplification product comprises
      i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
         (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
         (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
         (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
         (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
      ii. a fifth and a sixth restriction sites for nicking endonuclease arranged on opposite strands, wherein the fifth and sixth restriction sites are located outside a segment comprising the first inverted repeat at one end, the sequence of interest, and the second inverted repeat at the other end; and
   c. incubating the amplification product with a nicking endonuclease that nicks the fifth and sixth restriction sites to produce the precursor of the hairpin-ended DNA molecule comprising the first inverted repeat, the sequence of interest, and the second inverted repeat.
7. The method of paragraph 6, wherein the template comprises no additional restriction sites for nicking endonuclease, optionally wherein each of the fifth and sixth restriction sites for nicking endonuclease is present 1, 2, 3, 4, 5, 6, 7, 8, 9, 10 or more times in the template.
8. The method of paragraph 6 or 7, wherein (i) nicks created by nicking at the fifth and sixth restriction sites are 1, 2, 3, 4, 5, 6, 7, 8, 9, 10, 11, 12, 13, 14, 15, 16, 17, 18, 19, or 20 nucleotides apart; and (ii) single strand DNA overhangs created by nicking at the fifth and sixth restriction sites do not anneal at detectable levels inter- or intramolecularly under conditions that favor annealing of the first and/or second inverted repeat.
9 The method of any one of paragraphs 4 to 8, further comprising:
   a. incubating the precursor of the hairpin-ended DNA molecule with one or more nicking endonucleases recognizing the first, second, third, and fourth restriction site;
   b. denaturing and thereby creating a DNA fragment that comprises the two single strand DNA overhangs as specified in step b upon separation of the top from the bottom strand; and
   c. annealing the single strand DNA overhangs of the DNA fragment and thereby creating a hairpinned inverted repeat on each end of the DNA fragment resulting from the denaturing step to produce the hairpin-ended DNA molecule.
10. The method of paragraph 9, wherein the method produces non-hairpin-ended DNA molecules comprising at least one non-hairpin end, and the method further comprises digesting the non-hairpin-ended DNA molecules with an exonuclease, wherein the hairpin-ended DNA molecule is resistant to digestion by the exonuclease.
11. The method of any one of paragraphs 4-10, wherein the amplification product comprises an additional restriction enzyme site and/or additional restriction sites for nicking endonuclease located outside a segment comprising the first inverted repeat at one end, the sequence of interest, and the second inverted repeat at the other end, and the method further comprises creating additional non-hairpin-ended DNA molecules by cleaving the additional restriction enzyme site and/or nicking the additional restriction sites for nicking endonuclease.
12. A method for preparing a composition comprising pure hairpin-ended DNA molecules, wherein the method comprises:
   a. providing a circular DNA molecule as a template;
   b. incubating the template with a polymerase and a primer pair under conditions suitable for amplification to produce at least one amplification product and suitable for at least 2-fold amplification of the template, wherein the amplification product comprises
      i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
         (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
         (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
         (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
         (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
      ii. a restriction enzyme site wherein the restriction enzyme site is located outside a segment comprising the first inverted repeat at one end, the sequence of interest, and the second inverted repeat at the other end;
   c. incubating the amplification product with one or more nicking endonucleases recognizing the four restriction sites;
   d. denaturing and thereby creating a DNA fragment that comprises the two single strand DNA overhangs as specified in step b upon separation of the top from the bottom strand;
   e. annealing the single strand DNA overhangs and thereby creating a hairpinned inverted repeat on each end of the DNA fragment resulting from the denaturing step to produce a hairpin-ended DNA molecule comprising the sequence of interest and a hairpin-ended DNA molecule comprising the restriction enzyme site;
   f. incubating the hairpin-ended DNA molecule comprising the restriction enzyme site with a restriction enzyme that cleaves at the restriction enzyme site to produce a non-hairpin-ended DNA molecule comprising at least one non-hairpin end; and
   g. digesting the non-hairpin-ended DNA molecules with an exonuclease, wherein the hairpin-ended DNA molecule comprising the sequence of interest is resistant to digestion by the exonuclease.
13. The method of paragraph 12, wherein the template comprises no more than one of the restriction enzyme site.
14. A method for preparing a composition comprising pure hairpin-ended DNA molecules, wherein the method comprises:
   a. providing a circular DNA molecule as a template;
   b. incubating the template with a polymerase and a primer pair under conditions suitable for amplification to produce at least one amplification product and suitable for at least 2-fold amplification of the template, wherein the amplification product comprises
      i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
         (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
         (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
         (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
         (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
      ii. a fifth and a sixth restriction sites for nicking endonuclease arranged on opposite strands, wherein the fifth and sixth restriction sites are located outside a segment comprising the first inverted repeat at one end, the sequence of interest, and the second inverted repeat at the other end; and
   c. incubating the amplification product with one or more nicking endonucleases recognizing the four restriction sites;
   d. denaturing and thereby creating a DNA fragment that comprises the two single strand DNA overhangs as specified in step b upon separation of the top from the bottom strand;
   e. annealing the single strand DNA overhangs and thereby creating a hairpinned inverted repeat on each end of the DNA fragment resulting from the denaturing step to produce a hairpin-ended DNA molecule comprising the sequence of interest and a hairpin-ended DNA molecule comprising the fifth and sixth restriction site;
   f. incubating the hairpin-ended DNA molecule comprising the fifth and sixth restriction sites with a nicking endonuclease that nicks the fifth and sixth restriction sites to produce a non-hairpin-ended DNA molecule comprising at least one non-hairpin end; and
   g. digesting the non-hairpin-ended DNA molecules with an exonuclease, wherein the hairpin-ended DNA molecule comprising the sequence of interest is resistant to digestion by the exonuclease.
15. The method of paragraph 14, wherein (i) the nicks created by nicking at the fifth and sixth restriction sites are 1, 2, 3, 4, 5, 6, 7, 8, 9, 10, 11, 12, 13, 14, 15, 16, 17, 18, 19, or 20 nucleotides apart; and (ii) the single strand DNA overhangs created by nicking at the fifth and sixth restriction sites do not anneal at detectable levels inter- or intramolecularly under conditions that favor annealing of the first and/or second inverted repeat.
16. The method of any one of paragraphs 12-15, wherein the amplification product comprises an additional restriction enzyme site and/or additional restriction sites for nicking endonuclease located outside a segment comprising the first inverted repeat at one end, the sequence of interest, and the second inverted repeat at the other end, and the method further comprises creating additional non-hairpin-ended DNA molecules by cleaving the additional restriction enzyme site and/or nicking the additional restriction sites for nicking endonuclease.
17. A method for amplifying precursors of hairpin-ended DNA molecules, wherein the method comprises:
   a. providing a circular DNA molecule as a template comprising a methylated MSRE-recognition site; and
   b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
   c. incubating the template with an MSRE, wherein the amplification product comprises an unmethylated MSRE-recognition site located outside a segment comprising the first inverted repeat at one end, the sequence of interest, and the second inverted repeat at the other end, and the MSRE cleaves the amplification product at the unmethylated MSRE-recognition site.
18. The method of paragraph 17, wherein the circular DNA molecule is incubated with the polymerase and the MSRE concurrently, or the circular DNA molecule is incubated with the polymerase prior to the MSRE.
19. The method of paragraph 17 or 18, further comprising:
   a. incubating the MSRE-cleaved amplification products with one or more nicking endonucleases recognizing the four restriction sites, thereby creating the two single strand DNA overhangs as specified in step b upon separation of the top from the bottom strand;
   b. denaturing and thereby creating a DNA fragment that comprises the two single strand DNA overhangs; and
   c. annealing the single strand DNA overhangs and thereby creating a hairpinned inverted repeat on each end of the DNA fragment resulting from the denaturing step to produce a hairpin-ended DNA molecule.
20. A method for preparing hairpin-ended DNA molecules, wherein the method comprises:
   a. providing a circular DNA molecule comprising a methylated MSNE-restriction site;
   b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
   c. incubating the template with an MSNE, wherein the amplification product comprises two unmethylated MSNE-recognition sites located outside a segment comprising the first inverted repeat at one end, the sequence of interest, and the second inverted repeat at the other end, and the MSNE cleaves the amplification product at the two unmethylated MSNE-recognition sites.
21. The method of paragraph 20, wherein the circular DNA molecule is incubated with the polymerase and the MSNE concurrently, or the circular DNA molecule is incubated with the polymerase prior to the MSNE.
22. The method of paragraph 20 or 21, further comprising:
   a. incubating the MSNE-cleaved amplification products with one or more nicking endonucleases recognizing the four restriction sites, thereby creating the two single strand DNA overhangs as specified in step b upon separation of the top from the bottom strand;
   b. denaturing and thereby creating a DNA fragment that comprises the two single strand DNA overhangs; and
   c. annealing the single strand DNA overhangs and thereby creating a hairpinned inverted repeat on each end of the DNA fragment resulting from the denaturing step to produce a hairpin-ended DNA molecule.
23. A method for preparing hairpin-ended DNA molecules, wherein the method comprises:
   a. providing a circular DNA molecule as a template comprising a methylated MSRE-recognition site; and
   b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand;
   c. incubating the template with an MSRE, wherein the amplification product comprises an unmethylated MSRE-recognition site located outside the segment comprising the first inverted repeat at one end, the sequence of interest, and the second inverted repeat at the other end, and the MSRE cleaves the amplification product at the unmethylated MSRE-recognition site;
   d. incubating the MSRE-cleaved amplification products with one or more nicking endonucleases recognizing the four restriction sites, thereby creating the two single strand DNA overhangs as specified in step b upon separation of the top from the bottom strand;
   e. denaturing and thereby creating a DNA fragment that comprises the two single strand DNA overhangs; and
   f. annealing the single strand DNA overhangs and thereby creating a hairpinned inverted repeat on each end of the DNA fragment resulting from the denaturing step to produce a hairpin-ended DNA molecule.
24. The method of paragraph 23, wherein the method produces non-hairpin-ended DNA molecules comprising at least one non-hairpin end, and the method further comprises digesting the non-hairpin-ended DNA molecules with an exonuclease, wherein the hairpin-ended DNA molecule is resistant to digestion by the exonuclease.
25. A method for preparing hairpin-ended DNA molecules, wherein the method comprises:
   a. providing a circular DNA molecule comprising a methylated MSNE-restriction site;
   b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
   c. incubating the template with an MSNE, wherein the amplification product comprises two unmethylated MSNE-recognition sites located outside the segment comprising the first inverted repeat at one end, the sequence of interest, and the second inverted repeat at the other end, and the MSNE cleaves the amplification product at the two unmethylated MSNE-recognition sites;
   d. incubating the MSNE-cleaved amplification products with one or more nicking endonucleases recognizing the four restriction sites, thereby creating the two single strand DNA overhangs as specified in step b upon separation of the top from the bottom strand;
   e. denaturing and thereby creating a DNA fragment that comprises the two single strand DNA overhangs; and
   f. annealing the single strand DNA overhangs and thereby creating a hairpinned inverted repeat on each end of the DNA fragment resulting from the denaturing step to produce a hairpin-ended DNA molecule.
26. The method of paragraph 25, wherein (i) the nicks created by nicking at the two unmethylated restriction sites are 1, 2, 3, 4, 5, 6, 7, 8, 9, 10, 11, 12, 13, 14, 15, 16, 17, 18, 19, or 20 nucleotides apart; and (ii) the single strand DNA overhangs created by nicking at the two unmethylated restriction sites do not anneal at detectable levels inter- or intramolecularly under conditions that favor annealing of the first and/or second inverted repeat.
27. The method of paragraph 25 or 26, wherein the method further produces non-hairpin-ended DNA molecules comprising at least one non-hairpin end, and the method further comprises digesting the non-hairpin-ended DNA molecules with one or more exonucleases, wherein the hairpin-ended DNA molecule is resistant to digestion by the one or more exonucleases.
28. The method of any one of paragraphs 1 to 16, further comprising exchanging a buffer, concentrating the amplification product, and/or removing the circular DNA molecule, polymerase, and/or primer pair, after completion of step b and before initiation of step c.
29. The method of any one of paragraphs 17 to 27, further comprising exchanging a buffer, concentrating the amplification product, and/or removing the circular DNA molecule, polymerase, and/or primer pair, after completion of step c and before initiation of step d.
30. The method of paragraph any one of paragraphs 1 to 29, wherein the sequence of interest comprises a transcription unit encoding a therapeutic protein.
31. The method of any one of paragraphs 1 to 29, wherein the sequence of interest comprises a transcription unit encoding an RNA for in vitro transcription (IVT).
32. The method of any one of paragraphs 1 to 29, wherein the sequence of interest comprises a gene promoter, an AAV ITR, or a synthetic DNA template to be integrated into a genome.
33. The method of any one of paragraphs 1-32, wherein the circular DNA molecule is a single-stranded circular DNA molecule or a double stranded circular DNA molecule.
34. The method of any one of paragraphs 1-33, wherein the amplification is an isothermal amplification.
35. The method of paragraph 34, wherein the isothermal amplification is rolling circle amplification (RCA) and/or multiple displacement amplification (MDA).
36. A kit for preparing hairpin-ended DNA molecules, comprising:
   a. a circular DNA molecule as a template, wherein an amplification product amplified from the template comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand;
   b. a DNA polymerase suitable for amplification;
   c. a primer pair; and
   d. one or more nicking endonucleases recognizing the four restriction sites in the amplification product.
37. A kit for amplifying precursors of hairpin-ended DNA molecules, comprising:
   a. a circular DNA molecule as a template, wherein an amplification product amplified from the template comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand;
   b. a DNA polymerase suitable for amplification; and
   c. a primer pair.
38. The kit of paragraph 37, further comprising one or more nicking endonucleases recognizing the four restriction sites in the amplification product.
39. A kit for preparing precursors of hairpin-ended DNA molecules, comprising:
   a. a circular DNA molecule as a template, wherein an amplification product amplified from the template comprises:
      i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
         (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
         (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
         (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
         (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
      ii. a restriction enzyme site wherein the restriction enzyme site is located outside a segment comprising the first inverted repeat at one end, the sequence of interest, and the second inverted repeat at the other end;
   b. a DNA polymerase suitable for amplification;
   c. a primer pair; and
   d. a restriction enzyme that recognizes the restriction enzyme site.
40. A kit for preparing precursors of hairpin-ended DNA molecules, comprising:
   a. a circular DNA molecule as a template, wherein an amplification product amplified from the template comprises:
      i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
         (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
         (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
         (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
         (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
      ii. a fifth and a sixth restriction sites for nicking endonuclease arranged on opposite strands, wherein the fifth and sixth restriction sites are located outside a segment comprising the first inverted repeat at one end, the sequence of interest, and the second inverted repeat at the other end;
   b. a DNA polymerase suitable for amplification;
   c. a primer pair; and
   d. a nicking endonuclease that recognizes the fifth and a sixth restriction site.
41. The kit of paragraph 39 or 40, further comprising one or more nicking endonucleases recognizing the first, second, third, and forth restriction sites in the amplification product.
42. A kit for preparing a composition comprising pure hairpin-ended DNA molecules, comprising:
   a. a circular DNA molecule as a template, wherein an amplification product amplified from the template comprises:
      i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
         (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
         (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
         (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
         (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
      ii. a restriction enzyme site wherein the restriction enzyme site is located outside a segment comprising the first inverted repeat at one end, the sequence of interest, and the second inverted repeat at the other end;
   b. a DNA polymerase suitable for amplification;
   c. a primer pair;
   d. a restriction enzyme that recognizes the restriction enzyme site;
   e. one or more nicking endonucleases recognizing the first, second, third, and forth restriction sites in the amplification product; and
   f. an exonuclease.
43. A kit for preparing a composition comprising pure hairpin-ended DNA molecules, comprising:
   a. a circular DNA molecule as a template, wherein an amplification product amplified from the template comprises:
      i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
         (1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
         (2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
         (3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
         (4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
      ii. a fifth and a sixth restriction sites for nicking endonuclease arranged on opposite strands, wherein the fifth and sixth restriction sites are located outside a segment comprising the first inverted repeat at one end, the sequence of interest, and the second inverted repeat at the other end;
   b. a DNA polymerase suitable for amplification;
   c. a primer pair;
   d. a nicking endonuclease that recognizes the fifth and a sixth restriction site;
   e. one or more nicking endonucleases that recognizes the first, second, third, and forth restriction sites in the amplification product; and
   f. an exonuclease.
44. A kit for amplifying precursors of hairpin-ended DNA molecules, comprising:
   a. a circular DNA molecule as a template comprising a methylated MSRE-recognition site, wherein an amplification product amplified from the template comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
   b. an MSRE that recognizes and cleaves the amplification product at an unmethylated MSRE-recognition site located outside a segment comprising the first inverted repeat at one end, the sequence of interest, and the second inverted repeat at the other end;
   c. a DNA polymerase suitable for amplification; and
   d. a primer pair.
45. A kit for preparing hairpin-ended DNA molecules, comprising:
   a. a circular DNA molecule comprising a methylated MSNE-restriction site, wherein an amplification product amplified from the template comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
      i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
      ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
      iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
      iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
   b. an MSNE that recognizes and nicks the amplification product at the two unmethylated MSNE-recognition sites located outside a segment comprising the first inverted repeat at one end, the sequence of interest, and the second inverted repeat at the other end;
   c. a DNA polymerase suitable for amplification; and
   d. a primer pair.
46. The kit of paragraph 44 or 45, further comprising one or more nicking endonucleases that recognizes the first, second, third, and forth restriction sites in the amplification product.
47. The kit of paragraph 46, further comprising an exonuclease.
48. The kit of any one of paragraphs 39-47, wherein the amplification is an isothermal amplification.
49. The kit of paragraph 48, wherein the isothermal amplification is rolling circle amplification (RCA) and/or multiple displacement amplification (MDA).
50. A method of producing AAV vectors for use in gene therapy comprising:
   a. transfecting a host cell with at least one hairpin-ended DNA molecule for production of AAV particles, wherein the hairpin-ended DNA molecule has been produced according to the method of any one of paragraphs 1 to 36 and/or using the kit of any one of paragraphs 36 to 49; and
   b. harvesting the AAV particles.
51. The method of paragraph 50, wherein step a comprises co-transfecting the host cell with (i) a hairpin-ended DNA molecule encoding an AAV vector genome and (ii) one or more DNA molecules encoding Rep protein(s), AAV capsid protein(s), and/or helper plasmid(s).
52. The method of paragraph 50, wherein step a comprises co-transfecting the host cell with (i) a hairpin-ended DNA molecule encoding an AAV vector genome; (ii) a hairpin-ended DNA molecule encoding Rep proteins and AAV capsid proteins; and (iii) a hairpin-ended DNA molecule encoding helper plasmids.
53. A method of producing lentiviral vectors for use in gene therapy comprising:
   a. transfecting a host cell with at least one hairpin-ended DNA molecule for production of lentiviral particles, wherein the hairpin-ended DNA molecule has been produced according to the method of any one of paragraphs 1 to 36 and/or using the kit of any one of paragraphs 36 to 49; and
   b. harvesting the lentiviral particles.
54. The method of paragraph 53, wherein step a comprises co-transfecting the host cell with (i) a hairpin-ended DNA molecule encoding a lentiviral transfer vector and (ii) one or more DNA molecules encoding packaging and/or envelope proteins selected from the group consisting of VSV-G protein(s), Tat proteins, Rev protein(s), Gag protein(s), and Pol protein(s).
55. The method of paragraph 53, wherein step a comprises co-transfecting the host cell with (i) a hairpin-ended DNA molecule encoding a lentiviral transfer vector; (ii) a hairpin-ended DNA molecule encoding Rev protein; (iii) a hairpin-ended DNA molecule encoding Gag and Pol proteins; and (iv) a hairpin-ended DNA molecule encoding VSV-G protein.
56. A method of producing RNA comprising:
   a. transcribing a hairpin-ended DNA molecule, or a fragment thereof, for production of RNA, wherein the hairpin-ended DNA molecule comprises a transcription unit suitable for in vitro transcription (IVT) and has been produced according to the method of any one of paragraphs 1 to 36;
   b. harvesting the RNA product.
57. The method of paragraph 56, wherein the transcribing comprises the contacting the hairpin-ended DNA molecule, or fragment thereof, with an in vitro transcription reaction system comprising an RNA polymerase and ribonucleotides.

### 4. BRIEF DESCRIPTION OF THE DRAWINGS

FIGS. 1A-1C depict representative images of 1% agarose gels. FIG. 1A shows the digestion product after an amplification reaction. FIG. 1B shows the formation of hairpin-ended DNA. FIG. 1C shows the removal of non-hairpin ended DNA by an exonuclease.

FIGS. 2A-2B depict graphs showing the effect of increasing the Mg:dNTP ratio and KCl concentration on (FIG. 2A) final DNA concentration and (FIG. 2B) DNA amplification factors in amplification reactions.

FIGS. 3A-3B depict images and graphs showing cell transfection of amplified hairpin-ended DNA of the present disclosure. FIG. 3A depicts representative images of Huh-7 cells transfected with 17.5 fmol of amplified hairpin-ended DNA or its corresponding plasmid DNA template. Light grey-shaded areas correspond to EGFP-positive cells. Scale bar: 400 μm. FIG. 3B depicts the dose-response of firefly luciferase activity of cells transfected with the amplified hairpin-ended DNA of the present disclosure or its corresponding plasmid DNA template. RLU: relative luminescence units.

FIGS. 4A-4D depict representative images showing hairpin-ended DNA used for mRNA production from in vitro transcription (IVT) according to the present disclosure. FIG. 4A is a schematic of a genetic map of a hairpin-ended DNA used for mRNA production from IVT. FIGS. 4B-4D are representative images of 1% agarose gels showing (FIG. 4B) the digestion product after an amplification reaction, followed by (FIG. 4C) the formation of hairpin-ended DNA and subsequent exonuclease digest of non-hairpin-ended DNA, and (FIG. 4D) the purified hairpin-ended DNA for later use in IVT reactions to produce mRNA.

FIGS. 5A-5B provide a (FIG. 5A) representative 1% agarose gel image and (FIG. 5B) electropherograms (EPGs) of mRNA produced by in vitro transcription using a plasmid DNA template (pDNA) or amplified hairpin-ended DNA molecules (hpDNA).

FIGS. 6A-6B depict images and graphs showing cell transfection of amplified hairpin-ended DNA of the present disclosure. FIG. 6A depicts representative images of HEK293T cells transfected with 100 ng of mRNA from hairpin-ended DNA. Light grey-shaded areas correspond to EGFP-positive cells. Scale bar: 400 μm. FIG. 6B depicts firefly luciferase activity, as determined by the Bio-Glo Luciferase assay, for cells transfected with mRNA from hairpin-ended DNA. RLU: relative luminescence units.

FIG. 7 depicts a representative 1% agarose gel image of exonuclease V digestion of either amplified hairpin-ended DNA (lane 1) or a linear DNA (lane 2).

FIGS. 8A-8B depict Illumina read coverage of (FIG. 8A) an amplified hairpin-ended DNA and (FIG. 8B) its corresponding plasmid DNA template along the reference sequence. Highlighted areas show the ITRs (highlighted areas in FIG. 8A, and the first two highlighted areas in FIG. 8B) and the resistance gene (last highlighted area in FIG. 8B).

FIG. 9 depicts a representative image of a 1% agarose gel showing digestion of either a double-stranded circular template (“T”; lanes 2-3, 6, and 8) or precursors of hairpin-ended DNA (“hpDNA”; lanes 4-5, 7, and 9) by the MSREs ClaI (lanes 6-7) and BspDI (lanes 8-9). As controls, an undigested double-stranded circular template (lanes 2-3) and undigested precursors of hairpin-ended DNA (lanes 4-5) are shown.

FIGS. 10A-10D show the hairpin-ended DNA used for AAV production according to the present disclosure. FIG. 10A depicts a schematic of a genetic map of the hairpin-ended DNA for AAV production. FIGS. 10B-10D depict representative images of 1% agarose gels showing (FIG. 10B) the digestion product after amplification reaction, followed by (FIG. 10C) formation of hairpin-ended DNA, and (FIG. 10D) the subsequent exonuclease digest of non-hairpin-ended DNA.

FIGS. 11A-11C show characterization of AAV produced from amplified hairpin-ended DNA in combination with RepCap encoding for AAV9 and Helper plasmids. FIG. 11A depicts AAV yield, as determined by ITR2 qPCR. FIG. 11B depicts a representative image of a denaturing agarose gel showing the viral genome packaged in AAV particles. FIG. 11C depicts a representative image of a western blot showing the viral capsid proteins produced, and their relative abundancy.

FIG. 12 shows characterization of the DNA content of rAAV vectors produced from hairpin-ended DNA by next generation sequencing (NGS).

FIG. 13 shows the quantification of AAV9 preparation from triple transfection of amplified hairpin-ended DNA with Fecto VIR®-AAV. On the y axis the concentration of viral genome is shown as determined by transgene-specific qPCR.

FIG. 14A shows an electrophoresis for Cap1-mRNA with unmodified or modified nucleotides produced from hairpin-ended DNA and plasmid DNA template. FIG. 14B shows EGFP expression kinetics following transfection with unmodified or modified Cap1-mRNA synthesized with either hairpin-ended DNA or plasmid DNA template.

FIG. 15A shows a schematic representation of hairpin-ended DNA digestion sites with restriction enzymes. FIG. 15B shows an electrophoresis for Cap1-mRNA synthesized from hairpin-ended DNA digested with different enzymes. FIG. 15C shows EGFP expression kinetics following transfection with Cap1-mRNA synthesized from hairpin-ended DNA digested with different enzymes.

FIG. 16 depicts the transduction of HEK293T and Jurkat cells with lentiviral vectors (LVV) produced by HEK293T cells transfected with 4 amplified hairpin-ended DNA encoding CD19 CAR as sequence of interest, Gag/pol, Rev and VSV-G, respectively. FIG. 16A shows microscopy images of transduced HEK293T and Jurkat cells showing effective CD19-PE binding (depicted in grey). Mock LVV were produced by replacing hairpin-ended DNA encoding Gag/pol with a non-coding mock plasmid DNA, rendering production of LVV ineffective. Scale bar: 400 μm. FIG. 16B shows infectious viral titer of CD19 CAR LVV produced using hairpin-ended DNA, as determined by target cell transduction.

FIG. 17 depicts the usage of ClaI restriction endonuclease, a methylation-sensitive restriction enzyme (MSRE), on circular DNA substrates and within a DNA amplification process for production of hairpin-ended DNA to reduce overall viscosity of the reaction. FIG. 17A shows 1% agarose gel of DNA digestions using ClaI enzyme or Nt.BspQI enzyme on ~5 kB plasmid DNA extracted from bacteria, containing naturally methylated ClaI cutting sites compared to non-digested plasmid references (Ref). FIG. 17B shows dynamic viscosity of samples from a DNA amplification reaction performed according to the methods described in Example 1, with the same ~5 KB plasmid as starting template and at 30° C. Viscosity of samples after 3 h, 5.5 h, after addition of 0.1 U/uL ClaI enzyme and incubation for 0.5 h (=6.0 h of total amplification reaction) and after 1.5 h incubation after ClaI addition (=7.0 h of total amplification reaction) is shown.

### 5. DETAILED DESCRIPTION

Provided herein are methods and kits for making hairpin-ended DNA molecules and precursors thereof using a cell-free system through amplification of a circular DNA template, compositions comprising such made hairpin-ended DNA molecules, and uses thereof. Methods disclosed herein can produce transfection-ready high fidelity and high purity DNA molecules that are suitable for various uses (e.g., gene therapies). The DNA molecules are also transcription ready if the sequences of interest comprised in the DNA molecules encode in vitro transcribed (IVT) mRNAs.

In certain embodiments, methods disclosed herein comprise providing a circular DNA molecule as a template (see Section 5.1), and amplifying the template (see Section 5.2) to produce at least one amplification product (see Section 5.1). In certain embodiments, the amplification product is further processed to generate a hairpin-ended DNA molecule comprising a sequence of interest (see Section 5.3). In certain embodiments, undesired DNA molecules (e.g., non-hairpin-ended and hairpin-ended DNA molecules that do not comprise the sequence of interest) are removed from the reaction mixture to produce transfection/transcription-ready and high purity hairpin-ended DNA molecule (see Section 5.4).

In certain embodiments, the DNA molecules disclosed herein (e.g., DNA templates, amplification products amplified therefrom, and hairpin-ended DNA molecule produced therefrom) comprise at least two inverted repeats (see Section 5.1.1(a)). In certain embodiments, the DNA molecules comprise nicking endonuclease sites for creating single strand DNA overhangs comprising the inverted repeats (see Section 5.1.1(b)). In certain embodiments the DNA molecules comprise a sequence of interest (see Section 5.1.1(c)). Exemplary hairpin-ended DNA molecules made by the methods disclosed herein include the hairpin-ended DNA molecules disclosed in International Patent Publication No. WO 2022/023284, the content of which is incorporated by reference herein.

In certain embodiments, the amplification product amplified from the DNA template (see Section 5.2) is first processed to generate a precursor of the hairpin-ended DNA molecule (see Section 5.3.1). In certain embodiments, the precursor is further processed to generate the hairpin-ended DNA molecule (see Sections 5.3.2-5.3.5). In certain embodiments, the amplification product comprises a restriction enzyme site (see Section 5.1.3), and the precursor of the hairpin-ended DNA molecule is generated by incubating the amplification product with a restriction enzyme that cleaves at the restriction enzyme site (see Section 5.3.1). In certain embodiments, the amplification product comprises nicking endonuclease sites (see Section 5.1.4), where the precursor of the hairpin-ended DNA molecule is generated by incubating the amplification product with one or more nicking endonucleases that nick at the nicking endonuclease sites (see Section 5.3.1). In certain embodiments, processing the precursor of the hairpin-ended DNA molecule generates a hairpin-ended DNA molecule comprising the sequence of interest, and undesired non-hairpin-ended DNA molecules (e.g., DNA molecules comprising at least one non-hairpin end) (see Section 5.3.2-5.3.5).

In certain embodiments, the amplification product generated by the methods disclosed herein is directly processed to generate hairpin-ended DNA molecules (see Sections 5.3.2-5.3.5) without the step of generating a precursor of the hairpin-ended DNA molecule (see Section 5.3.1).

In certain embodiments, methods disclosed herein generate undesired DNA molecules, such as non-hairpin-ended and hairpin-ended DNA molecules that do not comprise the sequence of interest. Methods disclosed herein further comprise removing the undesired DNA molecules from the reaction mixture (see Section 5.4). In certain embodiments, the undesired DNA molecules (e.g., undesired hairpin-ended DNA molecules) comprise a restriction enzyme site (see Section 5.1.3), where the method further comprises incubating the undesired DNA molecules with a restriction enzyme that cleaves at the restriction enzyme site (see Section 5.4.1) to generate non-hairpin-ended DNA molecules. In certain embodiments, the undesired DNA molecules (e.g., undesired hairpin-ended DNA molecules) comprise nicking endonuclease sites (see Section 5.1.4), where the method further comprises incubating the undesired DNA molecules with one or more nicking endonucleases that nick at the nicking endonuclease sites (see Section 5.4.1) to generate non-hairpin-ended DNA molecules. In certain embodiments, the methods further comprise digesting the non-hairpin-ended DNA molecules with an exonuclease, whereas the hairpin-ended DNA molecule is resistant to the digestion by the exonuclease (see Section 5.4.2). Such digestion assists the removal of undesired DNA molecules from the reaction mix, and improves the purity of the generated hairpin-ended DNA molecules.

In certain embodiments, the DNA template amplification is further improved by using a methylation-sensitive restriction enzyme (MSRE) or a methylation-sensitive nicking endonuclease (MSNE), to cleave or nick the generated amplification products, which comprise an unmethylated MSRE or MSNE site (see Section 5.2.4). The DNA template remains intact, as the DNA template comprises a methylated MSRE or MSNE site, which is not cleavable or nickable by the MSRE or MSNE. Such process reduces the viscosity of the amplification products and improves the fidelity of the amplification.

In certain embodiments, DNA amplification process disclosed herein comprises continuously supplying components of the reaction (e.g., enzymes, polymerases, primers, dNTPs, and/or buffers) in batches or in a continuous flow, and thus is suitable for industrial scale production of the hairpin-ended DNA molecule (see Section 5.2.5).

In certain embodiments, the amplification product comprises a unmethylated MSRE site (see Section 5.1.3(a)), and the method comprises incubating the amplification product with an MSRE to cleave the amplification product at the MSRE site (see Section 5.2.4). In certain embodiments, the method further comprises processing the MSRE-cleaved amplification products to generate the hairpin-ended DNA molecules (see Sections 5.3.2-5.3.5). In certain embodiments, the amplification product comprises two unmethylated MSNE sites (see Section 5.1.4(a)), and the method comprises incubating the amplification product with one or more MSNEs to nick the amplification product at the MSNE sites (see Section 5.2.4). In certain embodiments, the method further comprises processing the MSNE-nicked amplification products to generate the hairpin-ended DNA molecules (see Sections 5.3.2-5.3.5). In certain embodiments, the methods further comprise digesting the non-hairpin-ended DNA molecules generated by MSRE or MSNE digestion with an exonuclease, whereas the hairpin-ended DNA molecule is resistant to the digestion by the exonuclease (see Section 5.4.2). The DNA amplification and MSRE/MSNE-mediated digestion can occur concurrently or the MSRE/MSNE can be added to the reaction mixture after the amplification is initiated, during the amplification, or after the amplification has ended.

As used herein and unless otherwise specified, the term “about” means within plus or minus 10% of a given value or range.

### 5.1 DNA Molecules

The present disclosure provides methods for generating hairpin-ended DNA molecules from amplification products, which are amplified from DNA templates, e.g., through isothermal amplification, e.g., RCA and/or MDA. DNA molecules provided herein include DNA templates, amplification products, and hairpin-ended DNA molecules disclosed herein.

In certain embodiments, the DNA template is a circular DNA. In certain embodiments, the DNA template is a double-stranded circular DNA. In certain embodiments, the DNA template is a single-stranded circular DNA.

In certain embodiments, the amplification products are double-stranded DNA molecules, which comprise at least one copy of the sequence of the DNA template. In certain embodiments, the amplification products are branched double-stranded DNA molecules, which comprise two or more copies of the sequence of the DNA template. In certain embodiments, the amplification products are further processed to generate hairpin-ended DNA molecules.

In certain embodiments, the DNA template comprises a primer binding site suitable for amplification of the DNA template. In certain embodiments, the amplification product comprises a primer binding site suitable for amplification to generate a complementary strand from a single-stranded DNA (e.g., the single-stranded DNA generated from amplifying a single-stranded circular DNA template). Primer binding site(s) for said amplification are described in Section 5.1.2 below.

In certain embodiments, the DNA template and amplification product comprise sequences forming the hairpin-ended DNA molecule. In certain embodiments, the sequences forming the hairpin-ended DNA molecule comprise inverted repeats from which the hairpin ends are formed (see Section 5.1.1(a)), nicking endonuclease sites for creating single strand DNA overhangs (see Section5.1.1(b)), and a sequence of interest (see Section 5.1.1(c)).

The DNA template and the amplification product can further comprise a restriction enzyme site (see Section 5.1.3) or additional nicking endonuclease sites (see Section 5.1.4) to create double strand breaks in the generated amplification product, for producing precursors of the hairpin-ended DNA molecules (see Section 5.3.1), producing double strand breaks for exonuclease digestion (see Section 5.4.1 and Section 5.4.2), and/or for reducing viscosity and improving fidelity of the amplification (e.g., isothermal amplification, e.g., RCA and MDA) (see Section 5.2.4).

In certain embodiments, the DNA template and/or amplification product disclosed herein is a double-stranded DNA molecule comprising in 5′ to 3′ direction of the top strand: i) a first inverted repeat (e.g., as described in Section 5.1.1(a)), wherein a first and a second restriction sites for nicking endonuclease are arranged on opposite strands in proximity of the first inverted repeat such that nicking results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g., as described in Sections 5.1.1(b), 5.3.3, and 5.3.4); ii) a sequence of interest (e.g., as described in Section 5.1.1(c)); and iii) a second inverted repeat (e.g., as described in Section 5.1.1(b)), wherein a third and a fourth restriction sites for nicking endonuclease are arranged on opposite strands in proximity of the second inverted repeat such that nicking results in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g., as described in Sections 5.1.1(b), 5.3.3, and 5.3.4). In certain embodiments, the top strand 5′ overhang comprises the first inverted repeat. In certain embodiments, the top strand 3′ overhang comprises the second inverted repeat. In certain embodiments, the top strand 5′ overhang comprises the first inverted repeat and the top strand 3′ overhang comprises the second inverted repeat.

In certain embodiments, the DNA template and/or amplification product disclosed herein is a double strand DNA molecule comprising in 5′ to 3′ direction of the top strand: i) a first inverted repeat (e.g., as described in Section 5.1.1(a)), wherein a first and a second restriction sites for nicking endonuclease are arranged on opposite strands in proximity of the first inverted repeat such that nicking results in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g., as described in Sections 5.1.1(b), 5.3.3, and 5.3.4); ii) a sequence of interest (e.g., as described in Section 5.1.1(c)); and iii) a second inverted repeat (e.g., as described in Section 5.1.1(a)), wherein a third and a fourth restriction sites for nicking endonuclease are arranged on opposite strands in proximity of the second inverted repeat such that nicking results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g., as described in Sections 5.1.1(b), 5.3.3, and 5.3.4). In certain embodiments, the bottom strand 3′ overhang comprises the first inverted repeat. In certain embodiments, the bottom strand 5′ overhang comprises the second inverted repeat. In certain embodiments, the bottom strand 3′ overhang comprises the first inverted repeat and the bottom strand 5′ overhang comprises the second inverted repeat.

In certain embodiments, the DNA template and/or amplification product disclosed herein is a double-stranded DNA molecule comprising in 5′ to 3′ direction of the top strand: i) a first inverted repeat (e.g., as described in Section 5.1.1(a)), wherein a first and a second restriction sites for nicking endonuclease are arranged on opposite strands in proximity of the first inverted repeat such that nicking results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g., as described in Sections 5.1.1(b), 5.3.3, and 5.3.4); ii) a sequence of interest (e.g., as described in Section 5.1.1(c)); and iii) a second inverted repeat (e.g., as described in Section 5.1.1(a)), wherein a third and a fourth restriction sites for nicking endonuclease are arranged on opposite strands in proximity of the second inverted repeat such that nicking results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g., as described in Sections 5.1.1(b), 5.3.3, and 5.3.4). In certain embodiments, the top strand 5′ overhang comprises the first inverted repeat. In certain embodiments, the bottom strand 5′ overhang comprises the second inverted repeat. In certain embodiments, the top strand 5′ overhang comprises the first inverted repeat and the bottom strand 5′ overhang comprises the second inverted repeat.

In certain embodiments, the DNA template and/or amplification product disclosed herein is a double strand DNA molecule comprising in 5′ to 3′ direction of the top strand: i) a first inverted repeat (e.g., as described in Section 5.1.1(a)), wherein a first and a second restriction sites for nicking endonuclease are arranged on opposite strands in proximity of the first inverted repeat such that nicking results in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g., as described in Sections 5.1.1(b), 5.3.3, and 5.3.4); ii) a sequence of interest (e.g., as described in Section 5.1.1(c)); and iii) a second inverted repeat (e.g., as described in Section 5.1.1(a)), wherein a third and a fourth restriction sites for nicking endonuclease are arranged on opposite strands in proximity of the second inverted repeat or a fragment thereof (e.g., at least 50%, 60%, 70%, 75%, 80%, 85%, 90%, 95%, or 99% of the second inverted repeat) such that nicking results in a top strand 3′ overhang comprising the second inverted repeat upon separation of the top from the bottom strand of the second inverted repeat (e.g., as described in Sections 5.1.1(b), 5.3.3, and 5.3.4). In certain embodiments, the bottom strand 3′ overhang comprises the first inverted repeat. In certain embodiments, the top strand 3′ overhang comprises the second inverted repeat. In certain embodiments, the bottom strand 3′ overhang comprises the first inverted repeat and the top strand 3′ overhang comprises the second inverted repeat.

In certain embodiments, the DNA template and/or amplification product disclosed herein is a double-stranded DNA molecule comprising in 5′ to 3′ direction of the top strand: i) a first inverted repeat (e.g., as described in Section 5.1.1(a)), wherein a first and a second target site for the guide nucleic acids for programmable nicking enzyme are arranged on opposite strands in proximity of the first inverted repeat such that nicking by programmable nicking enzyme results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g., as described in Sections 5.1.1(b), 5.3.3, and 5.3.4); ii) a sequence of interest (e.g., as described in Section 5.1.1(c)); and iii) a second inverted repeat (e.g., as described in Section 5.1.1(a)), wherein a third and a fourth target site for the guide nucleic acids for programmable nicking enzyme are arranged on opposite strands in proximity of the second inverted repeat such that nicking by programmable nicking enzyme results in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g., as described in Sections 5.1.1(b), 5.3.3, and 5.3.4). In certain embodiments, the top strand 5′ overhang comprises the first inverted repeat. In certain embodiments, the top strand 3′ overhang comprises the second inverted repeat. In certain embodiments, the top strand 5′ overhang comprises the first inverted repeat and the top strand 3′ overhang comprises the second inverted repeat.

In certain embodiments, the DNA template and/or amplification product disclosed herein is a double strand DNA molecule comprising in 5′ to 3′ direction of the top strand: i) a first inverted repeat (e.g., as described in Section 5.1.1(a)), wherein a first and a second target site for the guide nucleic acids for programmable nicking enzyme are arranged on opposite strands in proximity of the first inverted repeat such that nicking by programmable nicking enzyme results in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g., as described in Sections 5.1.1(b), 5.3.3, and 5.3.4); ii) a sequence of interest (e.g., as described in Section 5.1.1(c)); and iii) a second inverted repeat (e.g., as described in Section 5.1.1(a)), wherein a third and a fourth target site for the guide nucleic acids for programmable nicking enzyme are arranged on opposite strands in proximity of the second inverted repeat such that nicking by programmable nicking enzyme results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g., as described in Sections 5.1.1(b), 5.3.3, and 5.3.4). In certain embodiments, the bottom strand 3′ overhang comprises the first inverted repeat. In certain embodiments, the bottom strand 5′ overhang comprises the second inverted repeat. In certain embodiments, the bottom strand 3′ overhang comprises the first inverted repeat and the bottom strand 5′ overhang comprises the second inverted repeat.

In certain embodiments, the DNA template and/or amplification product disclosed herein is a double-stranded DNA molecule comprising in 5′ to 3′ direction of the top strand: i) a first inverted repeat (e.g., as described in Section 5.1.1(a)), wherein a first and a second target site for the guide nucleic acids for programmable nicking enzyme are arranged on opposite strands in proximity of the first inverted repeat such that nicking by programmable nicking enzyme results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g., as described in Sections 5.1.1(b), 5.3.3, and 5.3.4); ii) a sequence of interest (e.g., as described in Section 5.1.1(c)); and iii) a second inverted repeat (e.g., as described in Section 5.1.1(a)), wherein a third and a fourth target site for the guide nucleic acids for programmable nicking enzyme are arranged on opposite strands in proximity of the second inverted repeat such that nicking by programmable nicking enzyme results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g., as described in Sections 5.1.1(b), 5.3.3, and 5.3.4). In certain embodiments, the top strand 5′ overhang comprises the first inverted repeat. In certain embodiments, the bottom strand 5′ overhang comprises the second inverted repeat. In certain embodiments, the top strand 5′ overhang comprises the first inverted repeat and the bottom strand 5′ overhang comprises the second inverted repeat.

In certain embodiments, the DNA template and/or amplification product disclosed herein is a double strand DNA molecule comprising in 5′ to 3′ direction of the top strand: i) a first inverted repeat (e.g., as described in Section 5.1.1(a)), wherein a first and a second target site for the guide nucleic acids for programmable nicking enzyme are arranged on opposite strands in proximity of the first inverted repeat such that nicking by programmable nicking enzyme results in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g., as described in Sections 5.1.1(b), 5.3.3, and 5.3.4); ii) a sequence of interest (e.g., as described in Section 5.1.1(c)); and iii) a second inverted repeat (e.g., as described in Section 5.1.1(a)), wherein a third and a fourth target site for the guide nucleic acids for programmable nicking enzyme are arranged on opposite strands in proximity of the second inverted repeat such that nicking by programmable nicking enzyme results in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g., as described in Sections 5.1.1(b), 5.3.3, and 5.3.4). In certain embodiments, the bottom strand 3′ overhang comprises the first inverted repeat. In certain embodiments, the top strand 3′ overhang comprises the second inverted repeat. In certain embodiments, the bottom strand 3′ overhang comprises the first inverted repeat and the top strand 3′ overhang comprises the second inverted repeat. In certain embodiments, the first, second, third, and fourth target site for programmable nicking enzyme in this and the preceding three paragraphs are all the same. In certain embodiments, three of the first, second, third, and fourth target site for programmable nicking enzyme in this and the preceding three paragraphs are the same. In certain embodiments, two of the first, second, third, and fourth target site for programmable nicking enzyme in this and the preceding three paragraphs are the same. In a further embodiment, the first, second, third, and fourth target site for programmable nicking enzyme in this and the preceding three paragraphs are all different.

The DNA molecules provided herein comprise various features and have various embodiments as described in Section 3 and the preceding paragraphs of Section 5.1, which features and embodiments are further described in the various subsections below: the embodiments for the inverted repeats, including the first inverted repeat and/or the second inverted repeat, are described in Section 5.1.1(a), the embodiments for the restriction enzymes, nicking endonucleases, and their respective restriction sites are described in Sections 5.1.1(b) and 5.3.2, the embodiments for the programmable nicking enzymes and their targeting sites are described in Section 5.3.2, and the embodiments for the expression cassette are described in Section 5.1.1(c). As such, the disclosure provides DNA molecules comprising any permutations and combinations of the various embodiments of DNA molecules and embodiments of features of the DNA molecules described herein. In certain embodiments, the arrangement among the ITR, the sequence of interest, the restriction sites for nicking endonuclease or restriction enzymes, and the programmable nicking enzyme and their targeting sites can be any arrangement as described in Sections 5.3.2-5.3.4 and 5.1.1(a)-5.1.1(d).

In certain embodiments, the DNA template and/or amplification product disclosed herein is a double-stranded DNA molecule comprising in the 5′ to 3′ direction of the top strand: i) a first viral replication deficient inverted repeat (e.g., as described in Sections 5.1.1(a) and 5.1.5), wherein a first and a second restriction sites for nicking endonuclease are arranged on opposite strands in proximity of the first inverted repeat such that nicking results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g., as described in Sections 5.1.1(b), 5.3.3, and 5.3.4); ii) a sequence of interest (e.g., as described in Section 5.1.1(c)); and iii) a second viral replication deficient inverted repeat (e.g., as described in Sections 5.1.1(a) and 5.1.5), wherein a third and a fourth restriction sites for nicking endonuclease are arranged on opposite strands in proximity of the second inverted repeat such that nicking results in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g., as described in Sections 5.1.1(b), 5.3.3, and 5.3.4). In certain embodiments, the top strand 5′ overhang comprises the first viral replication deficient inverted repeat. In certain embodiments, the top strand 3′ overhang comprises the second viral replication deficient inverted repeat. In certain embodiments, the top strand 5′ overhang comprises the first viral replication deficient inverted repeat and the top strand 3′ overhang comprises the second viral replication deficient inverted repeat.

In certain embodiments, the DNA template and/or amplification product disclosed herein is a double strand DNA molecule comprising in the 5′ to 3′ direction of the top strand: i) a first viral replication deficient inverted repeat (e.g., as described in Sections 5.1.1(a) and 5.1.5), wherein a first and a second restriction sites for nicking endonuclease are arranged on opposite strands in proximity of the first inverted repeat such that nicking results in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g., as described in Sections 5.1.1(b), 5.3.3, and 5.3.4); ii) a sequence of interest (e.g., as described in Section 5.1.1(c)); and iii) a second viral replication deficient inverted repeat (e.g., as described in Sections 5.1.1(a) and 5.1.5), wherein a third and a fourth restriction sites for nicking endonuclease are arranged on opposite strands in proximity of the second inverted repeat such that nicking results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g., as described in Sections 5.1.1(b), 5.3.3, and 5.3.4). In certain embodiments, the bottom strand 3′ overhang comprises the first viral replication deficient inverted repeat. In certain embodiments, the bottom strand 5′ overhang comprises the second viral replication deficient inverted repeat. In certain embodiments, the bottom strand 3′ overhang comprises the first viral replication deficient inverted repeat and the bottom strand 5′ overhang comprises the second viral replication deficient inverted repeat.

In certain embodiments, the DNA template and/or amplification product disclosed herein is a double-stranded DNA molecule comprising in the 5′ to 3′ direction of the top strand: i) a first viral replication deficient inverted repeat (e.g., as described in Sections 5.1.1(a) and 5.1.5), wherein a first and a second restriction sites for nicking endonuclease are arranged on opposite strands in proximity of the first inverted repeat such that nicking results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g., as described in Sections 5.1.1(b), 5.3.3, and 5.3.4); ii) a sequence of interest (e.g., as described in Section 5.1.1(c)); and iii) a second viral replication deficient inverted repeat (e.g., as described in Sections 5.1.1(a) and 5.1.5), wherein a third and a fourth restriction sites for nicking endonuclease are arranged on opposite strands in proximity of the second inverted repeat such that nicking results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g., as described in Sections 5.1.1(b), 5.3.3, and 5.3.4). In certain embodiments, the top strand 5′ overhang comprises the first viral replication deficient inverted repeat. In certain embodiments, the bottom strand 5′ overhang comprises the second viral replication deficient inverted repeat. In certain embodiments, the top strand 5′ overhang comprises the first viral replication deficient inverted repeat and the bottom strand 5′ overhang comprises the second viral replication deficient inverted repeat.

In certain embodiments, the DNA template and/or amplification product disclosed herein is a double stranded DNA molecule comprising in 5′ to 3′ direction of the top strand: i) a first viral replication deficient inverted repeat (e.g., as described in Section 5.1.1(a) and 5.1.5), wherein a first and a second restriction sites for nicking endonuclease are arranged on opposite strands in proximity of the first inverted repeat such that nicking results in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g., as described in Sections 5.1.1(b), 5.3.3, and 5.3.4); ii) a sequence of interest (e.g., as described in Section 5.1.1(c)); and iii) a second viral replication deficient inverted repeat (e.g., as described in Sections 5.1.1(a) and 5.1.5), wherein a third and a fourth restriction sites for nicking endonuclease are arranged on opposite strands in proximity of the second inverted repeat or a fragment thereof (e.g., at least 50%, 60%, 70%, 75%, 80%, 85%, 90%, 95%, or 99% of the second inverted repeat) such that nicking results in a top strand 3′ overhang comprising the second inverted repeat upon separation of the top from the bottom strand of the second inverted repeat (e.g., as described in Sections 5.1.1(b), 5.3.3, and 5.3.4). In certain embodiments, the bottom strand 3′ overhang comprises the first viral replication deficient inverted repeat. In certain embodiments, the top strand 3′ overhang comprises the second viral replication deficient inverted repeat. In certain embodiments, the bottom strand 3′ overhang comprises the first viral replication deficient inverted repeat and the top strand 3′ overhang comprises the second viral replication deficient inverted repeat.

In certain embodiments, the hairpin-ended DNA molecule is of at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% purity in a composition disclosed herein. In certain embodiments, the hairpin-ended DNA molecule is of about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% purity in a composition disclosed herein. Certain embodiments of the hairpin-ended DNA molecules provided herein and compositions comprising said hairpin-ended DNA molecules in terms of purities are further described in Section 5.1.1(f), which can be combined in any suitable combination with the embodiments provided in this paragraph. In certain embodiments, the hairpin-ended DNA molecules are purified or isolated further from the reaction mixture disclosed herein. In certain embodiments, the hairpin-ended DNA molecules are not purified or isolated further from the reaction mixture disclosed herein.

The DNA molecules disclosed herein can lack certain sequences or features as further described in Section 5.1.5.

### 5.1.1 Hairpin-ended DNA Molecules

Hairpin-ended DNA molecules made by the methods disclosed herein include the hairpin-ended DNA molecules disclosed in International Patent Publication No. WO 2022/023284, the content of which is incorporated by reference herein. The present disclosure provides a cell-free manufacture of such hairpin-ended DNA molecules, which are transfection-ready. Some of the elements described below are present in the DNA template and amplification product from which the hairpin-ended DNA molecules are generated but may then no longer present in the resulting hairpin-ended DNA molecules.

### (a) Inverted Repeats

In certain embodiments, the sequences forming part of the hairpin-ended DNA molecule comprise inverted repeats from which the hairpin ends are formed. In certain embodiments, the DNA templates disclosed herein and/or amplification products produced therefrom comprise the inverted repeats disclosed herein.

“Inverted repeat” or “IR” refers to a single stranded nucleic acid sequence that comprises a palindromic sequence region. This palindromic region comprises a sequence of nucleotides as well as its reverse complement, i.e., “palindromic sequence”, on the same strand. In certain embodiments, the IR is an inverted terminal repeat (ITR). In certain embodiments, the IR comprise an ITR. In certain embodiments the IR can be a hairpinned inverted repeat. In certain embodiments, an inverted repeat, once folded upon itself, can create a hairpin loop (also known as stem loop) in which an unpaired loop of single stranded DNA is created when the DNA strand folds and forms base pairs with another section of the same strand. Upon folding, an inverted repeat can comprise one, two, three, four, five, six, seven, eight, nine, or ten such hairpin loop structures.

“Inverted terminal repeat” or “ITR” refers to an inverted repeat region that is at or proximal to a terminal of a single strand DNA molecule or an inverted repeat that is at or in the single strand overhang of a dsDNA molecule. An ITR can fold onto itself as a result of the palindromic sequence in the ITR. In certain embodiments, an ITR is at or proximal to one end of single-stranded DNA (ssDNA) or double-stranded DNA (dsDNA). In certain embodiments, two ITRs are each at or proximal to the two respective ends of an ssDNA or a dsDNA. In certain embodiments, the non-ITR part of the ssDNA or dsDNA comprises sequence(s) that are heterologous or homologous to the ITR. In a denatured state, the ITR comprising nucleic acid sequence is present in a random coil state (e.g., at high temperature, presence of chemical agents, high pH). In certain embodiments, as conditions become more suitable for annealing as described in Section 5.3.4, the ITR can fold on itself into a structure that is non-covalently held together by base pairing while the heterologous non-ITR part of the dsDNA remain intact or the heterologous non-ITR part of the ssDNA molecule can hybridize with a second ssDNA molecule comprising the reverse complement sequence of the heterologous DNA molecule. The resulting complex of two hybridized DNA strands encompass three distinct regions, a first folded single stranded ITR covalently linked to a double stranded DNA region that is in turn covalently linked to a second folded single stranded ITR. In certain embodiments, the ITR sequence can start at one of the restriction site for nicking endonuclease described in Sections 5.1.1(b) and end at the last base before the dsDNA. In certain embodiments, as opposed to a linear double stranded DNA molecule, the ITR present at the 5′ and 3′ termini of the top and bottom strand at either end of the DNA molecule can fold in and face each other (e.g., 3′ to 5′, 5′ to 3′ or vice versa) and therefore do not expose a free 5′ or 3′ terminus at either end of the nucleic acid duplex. When the ITR folds on itself, the dsDNA in the folded ITR can be immediately next to the dsDNA of the non-ITR part of the DNA molecule, creating a nick flanked by dsDNA in certain embodiments, or the dsDNA in the folded ITR can be one or more nucleotide apart from the dsDNA of the non-ITR part of the DNA molecule, creating a “ssDNA gap” flanked by dsDNA in certain embodiments. The two ITRs that flank the non-ITR DNA sequence are referred to an “ITR pair”. In certain embodiments, when the ITR assumes its folded state, it is resistant to exonuclease digestion (e.g., exonuclease V), e.g., for over an hour at 37° C.

The boundary between the terminal base of the ITR folded into its secondary structure and the terminal base of the DNA hybridized duplex can further be stabilized by stacking interactions (e.g., coaxial stacking) between base pairs flanking the nick or ssDNA gap and these interactions are sequence-dependent. In the case of a structure resembling a nick, an equilibrium between two conformations can exist wherein, the first conformation is very close to that of the intact double helix where stacking between the base pairs flanking the nick is conserved while the other conformation corresponds to complete loss of stacking at the nick site thus inducing a kink in DNA. Without being bound by theory, cellular proteins can recognize parallel 5′ and 3′ termini as double strand breaks and can engage as well as process these, which can adversely affect the fate of the DNA in a cell. Hence, the ITR can prevent premature, unwanted degradation of the presently disclosed hairpin-ended DNA molecules.

By placing a first and a second restriction sites for nicking endonucleases on opposite strands and in proximity of the inverted repeats and subsequent separation of the top from the bottom strand of the inverted repeat, the resulting overhang can fold back on itself and form a double stranded end that contains at least one restriction site for the nicking endonuclease. In certain embodiments, the folded ITR resembles the secondary structure conformation of viral ITRs. In certain embodiments, the ITR is located on both the 5′ and 3′ terminus of the bottom strand (e.g., a left ITR and right ITR). In certain embodiments, the ITR is located on both the 5′ and 3′ terminus of the top strand. In certain embodiments, one ITR is located at the 5′ terminus of the top strand, and the other ITR is located at the opposite end of the bottom strand (e.g., the left ITR at the 5′ terminus on the top strand and the right ITR at the 5′ terminus of the bottom). In certain embodiments, one ITR is located at the 3′ terminus of the top strand, and the other ITR is located at the 3′ terminus of the bottom strand.

In one aspect, the DNA template and/or amplification product disclosed herein comprise palindromic sequences. “Palindromic sequences” or “palindromes” are self-complimentary DNA sequences that can fold back to form a stretch of dsDNA in the self-complimentary region under a condition that favors intramolecular annealing. In certain embodiments, a palindromic sequence comprises a contiguous stretch of polynucleotides that is identical when read forwards as when read backwards on the complementary strand. In certain embodiments, a palindromic sequence comprises a stretch of polynucleotides that is identical when read forwards as when read backwards on the complementary strand, wherein such stretch is interrupted by one or more stretches of non-palindromic polynucleotides. In certain embodiments, a palindromic sequence comprises a stretch of polynucleotides that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical when read forwards as when read backwards on the complementary strand. In certain embodiments, a palindromic sequence comprises a stretch of polynucleotides that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical when read forwards as when read backwards on the complementary strand, wherein such stretch is interrupted by one or more stretches of non-palindromic polynucleotides. An ssDNA encoding one or more palindromic sequences can fold back upon itself, to form double stranded base pairs comprising a secondary structure (e.g., a hairpin loop, or a three-way junction).

Under appropriate conditions, for example as described in Section 5.3.4, an IR or an ITR provided herein can fold and form hairpin structures, including stems, a primary stem, loops, turning points, bulges, branches, branch loops, internal loops, and/or any combination or permutation of the structural features.

In certain embodiments, an IR or an ITR for the methods and compositions provided herein comprises one or more palindromic sequences. In certain embodiments, an IR or ITR described herein comprises palindromic sequences or domains that in addition to forming the primary stem domain can form branched hairpin structures. In certain embodiments, an IR or ITR comprises palindromic sequences that can form any number of branched hairpins. In certain specific embodiments, an IR or ITR comprises palindromic sequences that can form 1 to 30, or any subranges of 1 to 30, branched hairpins. In certain embodiments, an IR or ITR comprises palindromic sequences that can form 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 branched hairpins. In certain embodiments, an IR or ITR comprises sequence that can form two branched hairpin structures that lead to a three-way junction domain (T-shaped). In certain embodiments, an IR or ITR comprises sequence that can form three branched hairpin structures that lead to a four-way junction domain (or cruciform structure). In certain embodiments, an IR or ITR comprises sequence that can form a non-T-shaped hairpin structure, e.g., a U-shaped hairpin structure. In certain embodiments, an IR or ITR comprises sequence that can form interrupted U-shaped hairpin structure including a series of bulges and base pair mismatches. In certain embodiments, the branched hairpins all have the same length of stem and/or loop. In certain embodiments, one branched hairpin is smaller (e.g., truncated) than the other branched hairpins.

“Hairpin closing base pair” refers to the first base pair following the unpaired loop sequence. Certain stem loop sequences have preferred closing base pairs (e.g., GC in AAV2 ITRs). In certain embodiments, the stem loop sequence comprises G-C pair as the closing base pair. In certain embodiments, the stem loop sequence comprises C-G pair as the closing base pair.

“ITR closing base pair” refers to the first and last nucleotide that forms a base pair in a folded ITR. The terminal base pair is usually the pair of nucleotides of the primary stem domain that are most proximal to the non-ITR sequences (e.g., expression cassette) of the DNA molecule. The ITR closing base pair can be any type of base pair (e.g., CG, AT, GC or TA). In certain embodiments, the ITR closing base pair is a G-C base pair. In certain embodiments, the ITR closing base pair is an A-T base pair. In certain embodiments, the ITR closing base pair is a C-G base pair. In certain embodiments, the ITR closing base pair is a T-A base pair.

In certain embodiments, the ITR promotes the long-term survival of the nucleic acid molecule in the nucleus of a cell. In certain embodiments, the ITR promotes the permanent survival of the nucleic acid molecule in the nucleus of a cell (e.g., for the entire life-span of the cell). In certain embodiments, the ITR promotes the stability of the nucleic acid molecule in the nucleus of a cell. In certain embodiments, the ITR inhibits or prevents the degradation of the nucleic acid molecule in the nucleus of a cell.

In certain embodiments, IRs or ITRs can comprise any viral ITR. In certain embodiments, IRs or ITRs can comprise a synthetic palindromic sequence that can form a palindrome hairpin structure that does not expose a 5′ or 3′ terminus at the outmost apex or turning point of the repeat.

In certain embodiments, the single stranded ITR sequence stretching from one nucleotide of the ITR closing base pair to the other nucleotide of the ITR closing base pair has a Gibbs free energy (ΔG) of unfolding under physiological conditions in the range of −10 kcal/mol to −100 kcal/mol. In certain embodiments, the Gibbs free energy (ΔG) of unfolding referred to in the preceding sentence is no more than −10 (meaning≤−10, including e.g., −20, −30, etc.), no more than −11, no more than −12, no more than −13, no more than −14, no more than −15, no more than −16, no more than −17, no more than −18, no more than −19, no more than −20, no more than −21, no more than −22, no more than −23, no more than −24, no more than −25, no more than −26, no more than −27, no more than −28, no more than −29, no more than −30, no more than −31, no more than −32, no more than −33, no more than −34, no more than −35, no more than −36, no more than −37, no more than −38, no more than −39, no more than −40, no more than −41, no more than −42, no more than −43, no more than −44, no more than −45, no more than −46, no more than −47, no more than −48, no more than −49, no more than −50, no more than −51, no more than −52, no more than −53, no more than −54, no more than −55, no more than −56, no more than −57, no more than −58, no more than −59, no more than −60, no more than −61, no more than −62, no more than −63, no more than −64, no more than −65, no more than −66, no more than −67, no more than −68, no more than −69, no more than −70, no more than −71, no more than −72, no more than −73, no more than −74, no more than −75, no more than −76, no more than −77, no more than −78, no more than −79, no more than −80, no more than −81, no more than −82, no more than −83, no more than −84, no more than −85, no more than −86, no more than −87, no more than −88, no more than −89, no more than −90, no more than −91, no more than −92, no more than −93, no more than −94, no more than −95, no more than −96, no more than −97, no more than −98, no more than −99, or no more than −100 kcal/mol. In certain embodiments, the ΔG of unfolding referred to in the preceding sentence is about −10, about −11, about −12, about −13, about −14, about −15, about −16, about −17, about −18, about −19, about −20, about −21, about −22, about −23, about −24, about −25, about −26, about −27, about −28, about −29, about −30, about −31, about −32, about −33, about −34, about −35, about −36, about −37, about −38, about −39, about −40, about −41, about −42, about −43, about −44, about −45, about −46, about −47, about −48, about −49, about −50, about −51, about −52, about −53, about −54, about −55, about −56, about −57, about −58, about −59, about −60, about −61, about −62, about −63, about −64, about −65, about −66, about −67, about −68, about −69, about −70, about −71, about −72, about −73, about −74, about −75, about −76, about −77, about −78, about −79, about −80, about −81, about −82, about −83, about −84, about −85, about −86, about −87, about −88, about −89, about −90, about −91, about −92, about −93, about −94, about −95, about −96, about −97, about −98, about −99, or about −100 kcal/mol. In certain embodiments, the ITR sequence stretching from one nucleotide of the ITR closing base pair to the other nucleotide of the ITR closing base pair has a ΔG of unfolding under physiological conditions in the range of from −26 kcal/mol to −95 kcal/mol. In certain embodiments, the ITR sequence stretching from one nucleotide of the ITR closing base pair to the other nucleotide of the ITR closing base pair contribute to all of the ΔG of unfolding for the ITR sequence under physiological conditions.

In certain embodiments, in the folded state, the single stranded IR or ITR has an overall Watson-Crick self-complementarity of from about 50% to 98%. In certain embodiments, in the folded state, the single stranded IR or ITR has an overall Watson-Crick self-complementarity of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%. In certain embodiments, in the folded state, the single stranded IR or ITR has an overall Watson-Crick self-complementarity of at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In certain embodiments, in the folded state, IR or ITR has an overall Watson Crick complementarity of about 60% to 98%.

In certain embodiments, the single stranded IR or ITR has an overall GC content of between about 60 and 95%. In certain embodiments, the single stranded IR or ITR has an overall GC content of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95%. In certain embodiments, the single stranded IR or ITR has an overall GC content of about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%. In certain embodiments, the single stranded IR has an overall GC content of between about 60 and 91%.

Table 1 lists the folding free energy, GC content, percent of complementation, length of exemplary ITRs and lists the Sequences of the ITRs in Table 1.

TABLE 1
Folding free energy, GC content, percent of complementation, length of exemplary ITRs

| | | | | | Paired | GC | ΔG | Compl. | Unpaired |
|---|---|---|---|---|---|---|---|---|---|
| ITR | Length | A-T | G-C | G-T | Total | % | kcal/mol | % | % |
| SEQ ID NO: 1 | 85 | 8 | 31 | | 39 | 79% | −83.0 | 92% | 8% |
| SEQ ID NO: 2 | 77 | 7 | 28 | | 35 | 80% | −72.7 | 91% | 9% |
| SEQ ID NO: 3 | 69 | 5 | 26 | | 31 | 84% | −63.6 | 90% | 10% |
| SEQ ID NO: 4 | 89 | 7 | 34 | | 41 | 83% | −90.0 | 92% | 8% |
| SEQ ID NO: 5 | 71 | 6 | 26 | | 32 | 81% | −65.2 | 90% | 10% |
| SEQ ID NO: 6 | 59 | 4 | 22 | | 26 | 85% | −50.7 | 88% | 12% |
| SEQ ID NO: 7 | 51 | 2 | 20 | | 22 | 91% | −41.9 | 86% | 14% |
| SEQ ID NO: 8 | 70 | 7 | 13 | | 20 | 65% | −26.6 | 57% | 43% |
| SEQ ID NO: 10 | 92 | 6 | 18 | 1 | 25 | 75% | −52.1 | 52% | 48% |
| SEQ ID NO: 9 | 102 | 12 | 26 | | 38 | 68% | −72.8 | 75% | 25% |
| SEQ ID NO: 11 | 87 | 13 | 23 | | 36 | 64% | −63.0 | 83% | 17% |
| SEQ ID NO: 12 | 113 | 18 | 31 | | 49 | 63% | −93.6 | 87% | 13% |
| SEQ ID NO: 13 | 83 | 6 | 32 | | 38 | 84% | −83.0 | 92% | 8% |
| SEQ ID NO: 14 | 83 | 7 | 31 | | 38 | 82% | −80.0 | 92% | 8% |
| SEQ ID NO: 15 | 67 | 6 | 26 | | 32 | 81% | −79.1 | 96% | 4% |

TABLE 2
Sequences of the ITRs in Table 1

| SEQ ID NO | Sequence |
|---|---|
| SEQ ID NO: 1 | GCTCGACTCGCTCACTGAGGCCGGGCGACCAAAGGTCGCCCGACGCCCGG |
| | GCTTTGCCCGGGCGGCCTCAGTGAGCGAGTCGAGC |
| SEQ ID NO: 2 | GCTCGACTCACTGAGGCCGGGCGACCAAAGGTCGCCCGACGCCCGGGCTT |
| | TGCCCGGGCGGCCTCAGTGAGTCGAGC |
| SEQ ID NO: 3 | CGCTGACTCAGGCCGGGCGACCAAAGGTCGCCCGACGCCCGGGCTTTGCC |
| | CGGGCGGCCTGAGTCAGCG |
| SEQ ID NO: 4 | CGCGCTCGCTCGCTCACTGAGGCCGGGCGACCAAAGGTCGCCCGACGCCC |
| | GGGCTTTGCCCGGGCGGCCTCAGTGAGCGAGCGAGCGCG |
| SEQ ID NO: 5 | TCGCTCACTGAGGCCGGGCGACCAAAGGTCGCCCGACGCCCGGGCTTTGCC |
| | CGGGCGGCCTCAGTGAGCGA |
| SEQ ID NO: 6 | ACTGAGGCCGGGCGACCAAAGGTCGCCCGACGCCCGGGCTTTGCCCGGGC |
| | GGCCTCAGT |
| SEQ ID NO: 7 | AGGCCGGGCGACCAAAGGTCGCCCGACGCCCGGGCTTTGCCCGGGCGGCC |
| | T |
| SEQ ID NO: 8 | CCATGCATCCGGCTTTAAACGGGCAACTGCGTCTCATTCACGTTAGAGACT |
| | ACAACCGTCGGATGCATGG |
| SEQ ID NO: 9 | TTCAAACCTGCCGGGGGAGAAGCGGCGTTTTTTCCCGGCCGCCGCTTCTCT |
| | TCTTCTCCCGCCGCCGGGAAAAAAGGCGGGAGAAGCCCCGGCAGGTTTGA |
| | A |
| SEQ ID NO: 10 | GTCCGGGCCATGCTTCAAACCTGCCGGGGCTTCTCCCGCCTTTTTTCCCGGC |
| | GGCGGGAGAAGTAGATTTCTCGTACCTGCATGGCCCGGAC |
| SEQ ID NO: 11 | CCAGCGCTTGGGGTTGACGTGCCACTAAGATCAAGCGGCGCGCGCGCGCC |
| | GCTTGTCTTAGTGTCAAGGCAACCCCAAGCAAGCTGG |
| SEQ ID NO: 12 | GGTTGACTCTGGGCCAGCTTGCTTGGGGTTGCCTTGACACTAAGACAAGCG |
| | GCGCGCGCGCGCCGCTTGATCTTAGTGGCACGTCAACCCCAAGCGCTGGCC |
| | CAGAGTCAACC |
| SEQ ID NO: 13 | CGCGCTCGCTCGCTCACTGAGGCCGGGCCAAAGGCCCGACGCCCGGGCTTT |
| | GCCCGGGCGGCCTCAGTGAGCGAGCGAGCGCG |
| SEQ ID NO: 14 | CGCGCTCGCTCGCTCACTGAGGCCGGGCGACCAAAGGTCGCCCGACGCCC |
| | GTTTCGGGCGGCCTCAGTGAGCGAGCGAGCGCG |
| SEQ ID NO: 15 | CGCGCTCGCTCGCTCACTGAGGCCGCCCGGGCTTTGCCCGGGCGGCCTCAG |
| | TGAGCGAGCGAGCGCG |

The hairpin-ended DNA molecules made by methods disclosed herein can comprise IR or ITRs of various origins. In certain embodiments, the IR or ITR in the DNA molecule is a viral ITR. “Viral ITR” includes any viral terminal repeat or synthetic sequence that comprises at least one minimal required origin of replication and a region comprising a palindrome hairpin structure. In certain embodiments, the IR or ITR comprises functional elements capable of promoting the replication of the hairpin-ended DNA molecule in the nucleus of a cell. In certain embodiments, the IR or ITR comprises functional elements capable of promoting the packaging of the hairpin-ended DNA into a viral particle. In certain embodiments, the IR or ITR comprises functional elements capable of promoting the replication of the hairpin-ended DNA molecule in the nucleus of a cell and the packaging of the hairpin-ended DNA into a viral particle. In certain embodiments, the replication and/or packaging of the hairpin-ended DNA molecule are dependent on the presence and/or activity of viral and/or endogenous protein complexes. In certain embodiments, the IR or ITR is selected such that the hairpin-ended DNA molecule is “replication-ready” for the production of the viral vectors. In certain embodiments, the viral ITR is derived from Parvoviridae. In certain embodiments, the viral ITR derived from Parvoviridae comprises a minimal required origin of replication that comprises at least one viral replication-associated protein binding sequence (“RABS”). RABS refers to a DNA sequence to which a viral DNA replication-associated protein (“RAP”) or an isoform thereof, encoded by the Parvoviridae gene Rep and/or NS1, can bind. In certain embodiments, the RABS is a Rep binding sequence (“RBS”). In certain embodiments the RABS comprises a Rep binding sequence (“RBS”). Rep can bind to two elements within the ITR. It can bind to a nucleotide sequence in the stem structure of the ITR (i.e., the nucleotide sequence recognized by a Rep protein for replication of viral nucleic acid molecules). Such an RBS is also referred to as RBE (Rep-binding element). Rep can also bind to a nucleotide sequence, which forms a small palindrome comprising a single tip of an internal hairpin within the ITR, thereby stabilizing the association between Rep and the ITR. Such an RBS is also referred to as RBE′. In certain embodiments, the viral ITR derived from Parvoviridae comprises an RABS which comprises NS1-binding elements (“NSBEs”) that replication-associated viral protein NS1 can bind. In certain embodiments, the RABS is an NS1-binding element (“NSBE”) to which replication-associated viral protein NS1 can bind. In certain embodiments, viral ITR is derived from Parvoviridae and comprises a terminal resolution site (“TRS”) at which the viral DNA replication-associated proteins NS1 and/or Rep can perform an endonucleolytic nick within a sequence at the TRS. In certain embodiments, the viral ITR comprises at least one RBS or NSBE and at least one TRS. In the context of a virus or recombinant RAP (i.e., Rep or NS1) based production of viral genomes, the ITRs mediate replication and virus packaging. Hairpin-ended DNA molecules similar to viral ITRs can be produced without the need for Rep or NS1 proteins and consequently independent of the RABS or TRS sequence for DNA replication. Accordingly, the RABS and TRS can optionally be encoded in the nucleotide sequence disclosed herein but are not required and offer flexibility with regard to designing the ITRs. In certain embodiments, the ITR for the methods and compositions provided herein does not comprise at least one RABS (e.g., one RABS, two RABS, or more than two RABS). In certain embodiments, the ITR for the methods and compositions provided herein does not comprise any RABS. In certain embodiments, the ITR for the methods and compositions provided herein does not comprise at least one RBS. In certain embodiments, the ITR for the methods and compositions provided herein does not comprise any RBS. In certain embodiments, the ITR for the methods and compositions provided herein does not comprise RBE. In certain embodiments, the ITR for the methods and compositions provided herein does not comprise RBE′. In certain embodiments, the ITR for the methods and compositions provided herein does not comprise RBE and RBE′. In certain embodiments, the ITR for the methods and compositions provided herein does not comprise NSBE. In certain embodiments, the ITR for the methods and compositions provided herein does not comprise TRS. In a further embodiment, the ITR for the methods and compositions provided herein does not comprise at least one RABS (e.g., one RABS, two RABS, or more than two RABS) and does not comprise TRS. In a further embodiment, the ITR for the methods and compositions provided herein does not comprise any RABS and does not comprise TRS. In certain embodiments, the ITR for the methods and compositions provided herein comprises RBS (i.e., RBE and/or RBE′), TRS, or both RBS (i.e., RBE and/or RBE′) and TRS. In certain embodiments, the ITR for the methods and compositions provided herein comprises NBSE, TRS, or both NBSE and TRS.

“An ITR pair” refers to two ITRs within a single DNA molecule. In certain embodiments, the two ITRs in the ITR pair are both derived from wild type viral ITRs (e.g., AAV2 ITR) that have an inverse complement sequence across their entire length. An ITR can be considered to be a wild-type sequence, even if it has one or more nucleotides that deviate from the canonical naturally occurring sequence, so long as the changes do not affect the properties and overall three-dimensional structure of the sequence. The present disclosure provides that, in certain embodiments, the insertion, deletion or substitution of one or more nucleotides can provide the generation of a restriction site for nicking endonuclease without changing the overall three-dimensional structure of the viral ITR. In certain embodiments, the deviating nucleotides represent conservative sequence changes. In certain embodiments, the sequence of an ITR provided herein can have at least 95%, at least 96%, at least 97%, at least 98%, or at least 99% sequence identity to the canonical sequence (as measured, e.g., using BLAST at default settings), and also has a restriction site for nicking endonuclease, such that the 3D structures are the same shape in geometrical space. In certain embodiments, the sequence of an ITR provided herein can have about 95%, about 96%, about 97%, about 98%, or about 99% sequence identity to the canonical sequence (as measured, e.g., using BLAST at default settings), and also has a restriction site for nicking endonuclease, such that the 3D structures are the same shape in geometrical space.

In certain embodiments, a hairpin-ended DNA molecule made by methods disclosed herein comprises a pair of wildtype (wt)-ITRs. In certain specific embodiments, a hairpin-ended DNA molecule made by methods disclosed herein comprises a pair of wt-ITRs selected from the group shown in Table 3. Table 3 shows exemplary ITRs from the same serotype or different serotypes, or different parvoviruses, including AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12); AAVrh8, AAVrhlO, AAV-DJ, and AAV-DJ8 genome (e.g., NCBI: NC 002077; NC 001401; NC001729; NC001829; NC006152; NC 006260; NC 006261), ITRs from warm-blooded animals (avian AAV (AAAV), bovine AAV (BAAV), canine, equine, and ovine AAV), ITRs from B19 parvovirus (GenBank Accession No: NC 000883), Minute Virus from Mouse (MVM) (GenBank Accession No. NC 001510); Goose: goose parvovirus (GenBank Accession No. NC 001701); snake: snake parvovirus 1 (GenBank Accession No. NC 006148).

TABLE 3
Exemplary ITR sequences

| Virus | | |
|---|---|---|
| (accession | | |
| number) | Left ITR | Right ITR |
| AAV1 | TTGCCCACTCCCTCTCTGCGCGCTCG | TTGCCCACTCCCTCTCTGCGCGCTCGCT |
| | CTCGCTCGGTGGGGCCTGCGGACCA | CGCTCGGTGGGGCCTGCGGACCAAAGG |
| | AAGGTCCGCAGACGGCAGAGCTCTG | TCCGCAGACGGCAGAGCTCTGCTCTGCC |
| | CTCTGCCGGCCCCACCGAGCGAGCG | GGCCCCACCGAGCGAGCGAGCGCGCAG |
| | AGCGCGCAGAGAGGGAGTGGGCAA | AGAGGGAGTGGGCAA (SEQ ID NO: 17) |
| | (SEQ ID NO: 16) | |
| AAV2 | TTGGCCACTCCCTCTCTGCGCGCTCG | TTGGCCACTCCCTCTCTGCGCGCTCGCT |
| | CTCGCTCACTGAGGCCGGGCGACCA | CGCTCACTGAGGCCGGGCGACCAAAGG |
| | AAGGTCGCCCGACGCCCGGGCTTTG | TCGCCCGACGCCCGGGCTTTGCCCGGGC |
| | CCCGGGCGGCCTCAGTGAGCGAGCG | GGCCTCAGTGAGCGAGCGAGCGCGCAG |
| | AGCGCGCAGAGAGGGAGTGGCCAA | AGAGGGAGTGGCCAA (SEQ ID NO: 19) |
| | (SEQ ID NO: 18) | |
| AAV3 | TTGGCCACTCCCTCTATGCGCACTCG | TTGGCCACTCCCTCTATGCGCACTCGCT |
| | CTCGCTCGGTGGGGCCTGGCGACCA | CGCTCGGTGGGGCCTGGCGACCAAAGG |
| | AAGGTCGCCAGACGGACGTGCTTTG | TCGCCAGACGGACGTGCTTTGCACGTCC |
| | CACGTCCGGCCCCACCGAGCGAGCG | GGCCCCACCGAGCGAGCGAGTGCGCAT |
| | AGTGCGCATAGAGGGAGTGGCCAA | AGAGGGAGTGGCCAA (SEQ ID NO: 21) |
| | (SEQ ID NO: 20) | |
| AAV4 | TTGGCCACTCCCTCTATGCGCGCTCG | CTATGCGCGCTCGCTCACTCACTCGGCC |
| | CTCACTCACTCGGCCCTGGAGACCA | CTGGAGACCAAAGGTCTCCAGACTGCC |
| | AAGGTCTCCAGACTGCCGGCCTCTG | GGCCTCTGGCCGGCAGGGCCGAGTGAG |
| | GCCGGCAGGGCCGAGTGAGTGAGC | TGAGCGAGCGCGCATAGAGGGAGTGGC |
| | GAGCGCGCATAGAGGGAGTGGCCA | CAA (SEQ ID NO: 23) |
| | A (SEQ ID NO: 22) | |
| AAV5 | CTCTCCCCCCTGTCGCGTTCGCTCGC | CTCTCCCCCCTGTCGCGTTCGCTCGCTC |
| (NC_006152) | TCGCTGGCTCGTTTGGGGGGGTGGC | GCTGGCTCGTTTGGGGGGGTGGCAGCT |
| | AGCTCAAAGAGCTGCCAGACGACG | CAAAGAGCTGCCAGACGACGGCCCTCT |
| | GCCCTCTGGCCGTCGCCCCCCCAAA | GGCCGTCGCCCCCCCAAACGAGCCAGC |
| | CGAGCCAGCGAGCGAGCGAACGCG | GAGCGAGCGAACGCGACAGGGGGGAG |
| | ACAGGGGGGAGAG (SEQ ID NO: 24) | AG (SEQ ID NO: 25) |
| AAV7 | TTGGCCACTCCCTCTATGCGCGCTCG | TTGGCCACTCCCTCTATGCGCGCTCGCT |
| (NC_006260) | CTCGCTCGGTGGGGCCTGCGGACCA | CGCTCGGTGGGGCCTGCGGACCAAAGG |
| | AAGGTCCGCAGACGGCAGAGCTCTG | TCCGCAGACGGCAGAGCTCTGCTCTGCC |
| | CTCTGCCGGCCCCACCGAGCGAGCG | GGCCCCACCGAGCGAGCGAGCGCGCAT |
| | AGCGCGCATAGAGGGAGTGGCCAA | AGAGGGAGTGGCCAA (SEQ ID NO: 27) |
| | (SEQ ID NO: 26) | |
| HBOV | GTGGTTGTACAGACGCCATCTTGGA | TTGCTTATGCAATCGCGAAACTCTATAT |
| (JQ923422) | ATCCAATATGTCTGCCGGCTCAGTC | CTTTTAATGTGTTGTTGTTGTACATGCG |
| | ATGCCTGCGCTGCGCGCAGCGCGCT | CCATCTTAGTTTTATATCAGCTGGCGCC |
| | GCGCGCGCGCATGATCTAATCGCCG | TTAGTTATATAACATGCATGTTATATAA |
| | GCAGACATATTGGATTCCAAGATGG | CTAAGGCGCCAGCTGATATAAAACTAA |
| | CGTCTGTACAACCAC (SEQ ID NO: 28) | GATGGCGCATGTACAACAACAACACAT |
| | | TAAAAGATATAGAGTTTCGCGATTGCAT |
| | | AAGCAA (SEQ ID NO: 29) |
| hB19 | TGGGCCAGCTTGCTTGGGGTTGCCT | TGGGCCAGCGCTTGGGGTTGACGTGCC |
| (AY386330) | TGACACTAAGACAAGCGGCGCGCCG | ACTAAGATCAAGCGGCGCGCCGCTTGT |
| | CTTGATCTTAGTGGCACGTCAACCC | CTTAGTGTCAAGGCAACCCCAAGCAAG |
| | CAAGCGCTGGCCCA (SEQ ID NO: 30) | CTGGCCCA (SEQ ID NO: 31) |

In certain embodiments, the hairpin-ended DNA molecule comprises one or more ITRs derived from a wild-type AAV ITR (e.g., a wild-type AAV ITR listed in Table 3) by substitution, deletion, and/or addition of nucleotides in the nucleotide sequence of the wild-type AAV ITR. In certain embodiments, the one or more ITRs comprise derived from a wild-type AAV ITR comprise the nucleotide sequence of the wild-type AAV ITR (e.g. a nucleotide sequence listed in Table 3), and one or more nucleotides at the 5′ and/or 3′ end of the ITR. In certain embodiments, the hairpin-ended DNA molecule comprises of a pair of AAV ITRs derived from wt AAV2 ITRs. In certain embodiments, the hairpin-ended DNA molecule comprises a first ITR comprising a nucleotide sequence comprising SEQ ID NO 529, or a variant thereof, and a second ITR comprising a nucleotide sequence comprising SEQ ID NO 530, or a variant thereof. In certain embodiments, the hairpin-ended DNA molecule comprises a first ITR comprising a nucleotide sequence comprising SEQ ID NO 529 and a second ITR comprising a nucleotide sequence comprising SEQ ID NO 530. In certain embodiments, the hairpin-ended DNA molecule comprises a first ITR consisting of a nucleotide sequence comprising SEQ ID NO 529 and a second ITR consisting of a nucleotide sequence comprising SEQ ID NO 530. In certain embodiments, the hairpin-ended DNA molecule comprises a first ITR which is a variant of the nucleotide sequence of SEQ ID NO 529 and/or a second ITR which is a variant of the nucleotide sequence of SEQ ID NO 530. In certain embodiments, the variant of the DNA sequence of SEQ ID NO 529 and/or the variant of the nucleotide sequence of SEQ ID NO 530 is such that the D-loop of the ITR has been truncated or deleted. In certain embodiments, the variant of the nucleotide sequence of SEQ ID NO 529 and/or the variant of the nucleotide sequence of SEQ ID NO 530 is such that the TRS of the ITR has been mutated, truncated, or deleted. In certain embodiments, the variant of the nucleotide sequence of SEQ ID NO 529 and/or the variant of the nucleotide sequence of SEQ ID NO 530 is such the TRS and the D-loop of the ITR has been deleted. In certain embodiments, the variant of the nucleotide sequence of SEQ ID NO 529 and/or the variant of the nucleotide sequence of SEQ ID NO 530 is such the B-loop and the C-loop of the ITR have been swapped. In certain embodiments, the hairpin-ended DNA molecule comprises two variant ITRs.

In certain embodiments, the hairpin-ended DNA molecule comprises whole or part of the parvoviral genome. The parvoviral genome is linear, 3.9-6.3 kb in size, and the coding region is bracketed by terminal repeats that can fold into hairpin-like structures, which are either different (heterotelomeric, e.g., HBoV) or identical (homotelomeric, e.g., AAV2). In certain embodiments, the hairpin-ended DNA molecule comprises 2 different ITRs at the 2 ends of the DNA molecule. In certain embodiments, the hairpin-ended DNA molecule comprises 2 identical ITRs at the 2 ends of the DNA molecule. In certain embodiments, the hairpin-ended DNA molecule comprises 2 different ITRs at the 2 ends of the DNA molecule corresponding to the 2 HBoV ITRs. In certain embodiments the hairpin-ended DNA molecule comprises 2 identical ITRs at the 2 ends of the DNA molecule corresponding to the AAV2 ITR.

In certain embodiments, the ITR in the hairpin-ended DNA molecule can be an AAV ITR. In certain embodiments, the ITR can be a non-AAV ITR. In certain embodiments, the ITRs in the hairpin-ended DNA molecules can be derived from an AAV ITR or a non-AAV ITR. In certain embodiments, the ITR can be derived from any one of the family Parvoviridae, which encompasses parvoviruses and dependoviruses (e.g., canine parvovirus, bovine parvovirus, mouse parvovirus, porcine parvovirus, human parvovirus B-19). In certain embodiments, the ITR can be derived from the SV40 hairpin that serves as the origin of SV40 replication. Parvoviridae family viruses consist of two subfamilies: Parvovirinae, which infect vertebrates, and Densovirinae, which infect invertebrates. As such, in certain embodiments, the ITR can be derived from any one of the subfamily Parvovirinae. In certain embodiments, the ITR can be derived from any one of the subfamily Densovirinae.

In comparison to the T-shaped AAV ITRs, the human erythrovirus B19 has ITRs that terminate in imperfect, palindromes that can fold into long linear duplexes with a few unpaired nucleotides, creating a series of small, but highly conserved, mismatched bulges. In certain embodiments, any parvovirus ITR can be used as an ITR for the hairpin-ended DNA molecules (e.g., wild type or modified ITR) or can act as a template ITR for modification and then incorporation in the hairpin-ended DNA molecules. In certain embodiments, the parvovirus, from which the ITRs of the hairpin-ended DNA molecules are derived, is a dependovirus, an erythroparvovirus, or a bocaparvovirus. In certain embodiments, the ITRs of the hairpin-ended DNA molecules are derived from AAV, B19 or HBoV. In certain embodiments, the serotype of AAV ITRs chosen for the hairpin-ended DNA molecules can be based upon the tissue tropism of the serotype. AAV2 has a broad tissue tropism, AAV1 preferentially targets to neuronal and skeletal muscle, and AAV5 preferentially targets neuronal, retinal pigmented epithelia, and photoreceptors. AAV6 preferentially targets skeletal muscle and lung. AAV8 preferentially targets liver, skeletal muscle, heart, and pancreatic tissues. AAV9 preferentially targets liver, skeletal and lung tissue. In certain embodiments, the ITR or modified ITR of the hairpin-ended DNA molecules is based on an AAV2 ITR. In certain embodiments, the ITR or modified ITR of the hairpin-ended DNA molecules is based on an AAV1 ITR. In certain embodiments, the ITR or modified ITR of the hairpin-ended DNA molecules is based on an AAV5 ITR. In certain embodiments, the ITR or modified ITR of the hairpin-ended DNA molecules is based on an AAV6 ITR. In certain embodiments, the ITR or modified ITR of the hairpin-ended DNA molecules is based on an AAV8 ITR. In certain embodiments, the ITR or modified ITR of the hairpin-ended DNA molecules is based on an AAV9 ITR.

In certain embodiments, the hairpin-ended DNA molecules comprise at least one non-AAV ITR. In certain embodiments, such non-AAV ITR can be derived from hairpin sequences found in the mammalian genome. In certain embodiments, such non-AAV ITR can be derived from the hairpin sequences found in the mitochondrial genome including the OriL hairpin sequence (SEQ ID NO:32: 5′CTTCTCCCGCCGCCGGGAAAAAAGGCGGGAGAAGCCCCGGCAGGTTTGAA′3), which adopts a stem-loop structure and is involved in initiating the DNA synthesis of mitochondrial DNA (see Fuste et al., Molecular Cell, 37, 67-78, Jan. 15, 2010, which is incorporated herein in its entirety by reference). In certain embodiments, the hairpin-ended DNA molecules comprise an ITR derived from the OriL sequence that is mirrored to form a T junction with two self-complimentary palindromic regions and a 12-nucleotide loop at either apex of the hairpin. In certain embodiments, the hairpin-ended DNA molecules comprise an ITR derived from the OriL sequence that maintains OriL hairpin loop followed by an unpaired bulge and a GC-rich stem.

In certain embodiments, the hairpin-ended DNA molecules comprise one or more non-AAV ITRs that are derived from aptamer. Similar to viral ITRs, aptamers are composed of ssDNA that folds into a three-dimensional structure and have the ability to recognize biological targets with high affinity and specificity. DNA aptamers can be generated by systematic evolution of ligands by exponential enrichment (SELEX). For example, it has previously been shown that some aptamers can target the nuclei of human cells (See Shen et al ACS Sens. 2019, 4, 6, 1612-1618, which is herein incorporated in its entirety by reference). In certain embodiments, the hairpin-ended DNA molecules comprise nucleus targeting aptamer ITRs or their derivatives, wherein the aptamer specifically binds nuclear protein. In certain embodiments, the aptamer ITRs fold into a secondary structure that can contain such as hairpins as well as internal loops as well bulges and a stem region.

In certain embodiments, the hairpin-ended DNA molecules comprise one or more AAV2 ITR, human erythrovirus B19 ITR goose parvovirus ITR, and/or their derivatives in any combination. In certain embodiments, the hairpin-ended DNA molecules comprise two ITRs selected from AAV2 ITR, human erythrovirus B19 ITR goose parvovirus ITR, and their derivatives, in any combination. In certain embodiments, the hairpin-ended DNA molecules comprise one or more AAV2 ITR, human erythrovirus B19 ITR goose parvovirus ITR, and/or their derivatives, in any combination, wherein the ITRs remain functional regardless of whether the palindromic regions of their ITRs are in direct, reverse, or any possible combination of 5′ and 3′ ITR directionality with respect to the expression cassette (as described in WO2019143885, which is herein incorporated in its entirety by reference).

In certain embodiments, a modified IR or ITR in the hairpin-ended DNA molecules is a synthetic IR sequence that comprises a restriction site for endonuclease such as 5′-GAGTC-3′ (SEQ ID NO: 33) in addition to various palindromic sequence allowing for hairpin secondary structure formation as described in this Section (Section 5.1.1(a)).

In certain embodiments, the IR or ITR in the hairpin-ended DNA molecules can be an IR or ITR having various sequence homology with the IR or ITR sequences described in this Section (Section 5.1.1(a)). In certain embodiments, the IR or ITR in the hairpin-ended DNA molecules can be an IR or ITR having various sequence homology with the known IR or ITR sequences of various ITR origins described in this Section (Section 5.1.1(a)) (e.g., viral ITR, mitochondria ITR, artificial or synthetic ITR such as aptamers, etc.). In certain embodiments, such homology provided in this paragraph can be a homology of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In certain embodiments, such homology provided in this paragraph can be a homology of about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%.

In certain embodiments, the IR or ITR in the hairpin-ended DNA molecules can comprise any one or more features described in this Section (Section 5.1.1(a)), in various permutations and combinations.

### (b) Nicking Endonuclease Sites for Creating Single Strand DNA Overhangs

In certain embodiments, the DNA templates disclosed herein and/or amplification products produced therefrom comprise nicking endonuclease sites (i.e., restriction sites for nicking endonucleases) for creating single strand DNA overhangs. Exemplary nicking endonucleases that nick at nicking endonuclease sites are disclosed in Section 5.3.2. Alternatively, a programmable nicking enzyme can be used to nick and the presently disclosed restriction sites (see Section 5.3.2).

In certain embodiments, the first, second, third, and fourth restriction sites for nicking endonuclease comprised by the amplification products disclosed herein are targeted and nicked by the same nicking endonuclease. In certain embodiments, the first, second, third, and fourth restriction sites for nicking endonuclease comprised by the amplification products are targeted and nicked by two or more different nicking endonucleases, e.g., two, three or four different nicking endonucleases. In certain embodiments, each of the two or more different nicking endonucleases targets different restriction sites of the first, second, third, and fourth restriction sites.

In certain embodiments, the nicking endonuclease and restriction sites for the nicking endonuclease (e.g., the first, second, third, and fourth restriction sites) is selected from those described in Section 5.3.2 (e.g., Table 21).

Exemplary modified AAV ITR sequences that harbor two antiparallel recognition sites for the same nicking endonuclease, grouped by nicking endonuclease species are disclosed in Tables 7-16 of International Patent Publication No. WO 2022/023284 and are reproduced in Table 4-Table 13 below.

TABLE 4
Exemplary AAV derived ITRs harboring antiparallel recognition sites for nicking
endonuclease Nb.BvCI:

| SEQ ID NO | Name | Full Sequence |
|---|---|---|
| SEQ ID NO: 34 | source: AAV1; Recogn. | TTGCCCACTCCCCCTCAGCGCGCTCGCTCGCTCGGTGGGGCCTGCG |
| | Site: Nb.BbvCI; Format: | GACCAAAGGTCCGCAGACGGCAGAGCTCTGCTCTGCCGGCCCCACC |
| | bl | GAGCGAGCGAGCGCGCTGAGGGGGAGTGGGCAA |
| SEQ ID NO: 35 | source: AAV1; Recogn. | TTGCCCACTCCCGCTGAGGGCGCTCGCTCGCTCGGTGGGGCCTGCG |
| | Site: Nb.BbvCI; Format: | GACCAAAGGTCCGCAGACGGCAGAGCTCTGCTCTGCCGGCCCCACC |
| | tl | GAGCGAGCGAGCGCCCTCAGCGGGAGTGGGCAA |
| SEQ ID NO: 36 | source: AAV2; Recogn. | TTGGCCACTCCCCCTCAGCGCGCTCGCTCGCTCACTGAGGCCGGGC |
| | Site: Nb.BbvCI; Format: | GACCAAAGGTCGCCCGACGCCCGGGCTTTGCCCGGGCGGCCTCAGT |
| | bl | GAGCGAGCGAGCGCGCTGAGGGGGAGTGGCCAA |
| SEQ ID NO: 37 | source: AAV2; Recogn. | TTGGCCACTCCCGCTGAGGGCGCTCGCTCGCTCACTGAGGCCGGGC |
| | Site: Nb.BbvCI; Format: | GACCAAAGGTCGCCCGACGCCCGGGCTTTGCCCGGGCGGCCTCAGT |
| | t1 | GAGCGAGCGAGCGCCCTCAGCGGGAGTGGCCAA |
| SEQ ID NO: 38 | source: AAV3; Recogn. | TTGGCCACTCCCCCTCAGCGCACTCGCTCGCTCGGTGGGGCCTGGC |
| | Site: Nb.BbvCI; Format: | GACCAAAGGTCGCCAGACGGACGTGCTTTGCACGTCCGGCCCCACC |
| | bl | GAGCGAGCGAGTGCGCTGAGGGGGAGTGGCCAA |
| SEQ ID NO: 39 | source: AAV3; Recogn. | TTGGCCACTCCCGCTGAGGGCACTCGCTCGCTCGGTGGGGCCTGGC |
| | Site: Nb.BbvCI; Format: | GACCAAAGGTCGCCAGACGGACGTGCTTTGCACGTCCGGCCCCACC |
| | t1 | GAGCGAGCGAGTGCCCTCAGCGGGAGTGGCCAA |
| SEQ ID NO: 40 | source: AAV4 left; | TTGGCCACTCCCCCTCAGCGCGCTCGCTCACTCACTCGGCCCTGGA |
| | Recogn. Site: Nb.BbvCI; | GACCAAAGGTCTCCAGACTGCCGGCCTCTGGCCGGCAGGGCCGAGT |
| | Format: bl | GAGTGAGCGAGCGCGCTGAGGGGGAGTGGCCAA |
| SEQ ID NO: 41 | source: AAV4 left; | TTGGCCACTCCCGCTGAGGGCGCTCGCTCACTCACTCGGCCCTGGA |
| | Recogn. Site: Nb.BbvCI; | GACCAAAGGTCTCCAGACTGCCGGCCTCTGGCCGGCAGGGCCGAGT |
| | Format: tl | GAGTGAGCGAGCGCCCTCAGCGGGAGTGGCCAA |
| SEQ ID NO: 42 | source: AAV4 right; | TTGGCCACATTACCTCAGCGCGCTCGCTCACTCACTCGGCCCTGGA |
| | Recogn. Site: Nb.BbvCI; | GACCAAAGGTCTCCAGACTGCCGGCCTCTGGCCGGCAGGGCCGAGT |
| | Format: bl | GAGTGAGCGAGCGCGCTGAGGGGGAGTGGCCAA |
| SEQ ID NO: 43 | source: AAV4 right; | TTGGCCACATTAGCTGAGGGCGCTCGCTCACTCACTCGGCCCTGGA |
| | Recogn. Site: Nb.BbvCI; | GACCAAAGGTCTCCAGACTGCCGGCCTCTGGCCGGCAGGGCCGAGT |
| | Format: tl | GAGTGAGCGAGCGCCCTCAGCGGGAGTGGCCAA |
| SEQ ID NO: 44 | source: AAV5; Recogn. | CTCTCCCCTCAGCCGCGTTCGCTCGCTCGCTGGCTCGTTTGGGGGG |
| | Site: Nb.BbvCI; Format: | GTGGCAGCTCAAAGAGCTGCCAGACGACGGCCCTCTGGCCGTCGCC |
| | bl | CCCCCAAACGAGCCAGCGAGCGAGCGAACGCGGCTGAGGGGAGAG |
| SEQ ID NO: 45 | source: AAV5; Recogn. | CTCTCCCCGCTGAGGCGTTCGCTCGCTCGCTGGCTCGTTTGGGGGG |
| | Site: Nb.BbvCI; Format: | GTGGCAGCTCAAAGAGCTGCCAGACGACGGCCCTCTGGCCGTCGCC |
| | tl | CCCCCAAACGAGCCAGCGAGCGAGCGAACGCCTCAGCGGGGAGAG |
| SEQ ID NO: 46 | source: AAV7; Recogn. | TTGGCCACTCCCCCTCAGCGCGCTCGCTCGCTCGGTGGGGCCTGCG |
| | Site: Nb.BbvCI; Format: | GACCAAAGGTCCGCAGACGGCAGAGCTCTGCTCTGCCGGCCCCACC |
| | bl | GAGCGAGCGAGCGCGCTGAGGGGGAGTGGCCAA |
| SEQ ID NO: 47 | source: AAV7; Recogn. | TTGGCCACTCCCGCTGAGGGCGCTCGCTCGCTCGGTGGGGCCTGCG |
| | Site: Nb.BbvCI; Format: | GACCAAAGGTCCGCAGACGGCAGAGCTCTGCTCTGCCGGCCCCACC |
| | t1 | GAGCGAGCGAGCGCCCTCAGCGGGAGTGGCCAA |

TABLE 5
Exemplary AAV derived ITRs harboring antiparallel recognition sites for nicking
endonuclease Nb.BsmI

| SEQ ID NO | Name | Full Sequence |
|---|---|---|
| SEQ ID NO: 48 | source: AAV1; Recogn. | TTGCCCACTCCCTGAATGCGCGCTCGCTCGCTCGGTGGGGCCTGCGG |
| | Site: Nb.Bsml; Format: bl | ACCAAAGGTCCGCAGACGGCAGAGCTCTGCTCTGCCGGCCCCACCGA |
| | | GCGAGCGAGCGCGCATTCAGGGAGTGGGCAA |
| SEQ ID NO: 49 | source: AAV1; Recogn. | TTGCCCACTCCCTCTCTGCGCATTCGCTCGCTCGGTGGGGCCTGCGG |
| | Site: Nb.Bsml; Format: tl | ACCAAAGGTCCGCAGACGGCAGAGCTCTGCTCTGCCGGCCCCACCGA |
| | | GCGAGCGAATGCGCAGAGAGGGAGTGGGCAA |
| SEQ ID NO: 50 | source: AAV2; Recogn. | TTGGCCACTCCCTGAATGCGCGCTCGCTCGCTCACTGAGGCCGGGCG |
| | Site: Nb.Bsml; Format: bl | ACCAAAGGTCGCCCGACGCCCGGGCTTTGCCCGGGCGGCCTCAGTGA |
| | | GCGAGCGAGCGCGCATTCAGGGAGTGGCCAA |
| SEQ ID NO: 51 | source: AAV2; Recogn. | TTGGCCACTCCCTCTCTGCGCATTCGCTCGCTCACTGAGGCCGGGCG |
| | Site: Nb.Bsml; Format: tl | ACCAAAGGTCGCCCGACGCCCGGGCTTTGCCCGGGCGGCCTCAGTGA |
| | | GCGAGCGAATGCGCAGAGAGGGAGTGGCCAA |
| SEQ ID NO: 52 | source: AAV3; Recogn. | TTGGCCACTCCCTGAATGCGCACTCGCTCGCTCGGTGGGGCCTGGCG |
| | Site: Nb.Bsml; Format: bl | ACCAAAGGTCGCCAGACGGACGTGCTTTGCACGTCCGGCCCCACCGA |
| | | GCGAGCGAGTGCGCATTCAGGGAGTGGCCAA |
| SEQ ID NO: 53 | source: AAV3; Recogn. | TTGGCCACTCCCTCTATGCGCATTCGCTCGCTCGGTGGGGCCTGGCG |
| | Site: Nb.Bsml; Format: tl | ACCAAAGGTCGCCAGACGGACGTGCTTTGCACGTCCGGCCCCACCGA |
| | | GCGAGCGAATGCGCATAGAGGGAGTGGCCAA |
| SEQ ID NO: 54 | source: AAV4 left; | TTGGCCACTCCCTGAATGCGCGCTCGCTCACTCACTCGGCCCTGGAG |
| | Recogn. Site: Nb.Bsml; | ACCAAAGGTCTCCAGACTGCCGGCCTCTGGCCGGCAGGGCCGAGTGA |
| | Format: bl | GTGAGCGAGCGCGCATTCAGGGAGTGGCCAA |
| SEQ ID NO: 55 | source: AAV4 left; | TTGGCCACTCCCTCTATGCGCATTCGCTCACTCACTCGGCCCTGGAG |
| | Recogn. Site: Nb.Bsml; | ACCAAAGGTCTCCAGACTGCCGGCCTCTGGCCGGCAGGGCCGAGTGA |
| | Format: tl | GTGAGCGAATGCGCATAGAGGGAGTGGCCAA |
| SEQ ID NO: 56 | source: AAV4 right; | TTGGCCACATTAGGAATGCGCGCTCGCTCACTCACTCGGCCCTGGAG |
| | Recogn. Site: Nb.Bsml; | ACCAAAGGTCTCCAGACTGCCGGCCTCTGGCCGGCAGGGCCGAGTGA |
| | Format: bl | GTGAGCGAGCGCGCATTCAGGGAGTGGCCAA |
| SEQ ID NO: 57 | source: AAV4 right; | TTGGCCACATTAGCTATGCGCATTCGCTCACTCACTCGGCCCTGGAG |
| | Recogn. Site: Nb.Bsml; | ACCAAAGGTCTCCAGACTGCCGGCCTCTGGCCGGCAGGGCCGAGTGA |
| | Format: tl | GTGAGCGAATGCGCATAGAGGGAGTGGCCAA |
| SEQ ID NO: 58 | source: AAV5; Recogn. | CTCTCCCCGAATGCGCGTTCGCTCGCTCGCTGGCTCGTTTGGGGGGG |
| | Site: Nb.Bsml; Format: bl | TGGCAGCTCAAAGAGCTGCCAGACGACGGCCCTCTGGCCGTCGCCCC |
| | | CCCAAACGAGCCAGCGAGCGAGCGAACGCGCATTCGGGGAGAG |
| SEQ ID NO: 59 | source: AAV5; Recogn. | CTCTCCCCCCTGTCGCATTCGCTCGCTCGCTGGCTCGTTTGGGGGGG |
| | Site: Nb.Bsml; Format: tl | TGGCAGCTCAAAGAGCTGCCAGACGACGGCCCTCTGGCCGTCGCCCC |
| | | CCCAAACGAGCCAGCGAGCGAGCGAATGCGACAGGGGGGAGAG |
| SEQ ID NO: 60 | source: AAV7; Recogn. | TTGGCCACTCCCTGAATGCGCGCTCGCTCGCTCGGTGGGGCCTGCGG |
| | Site: Nb.Bsml; Format: bl | ACCAAAGGTCCGCAGACGGCAGAGCTCTGCTCTGCCGGCCCCACCGA |
| | | GCGAGCGAGCGCGCATTCAGGGAGTGGCCAA |
| SEQ ID NO: 61 | source: AAV7; Recogn. | TTGGCCACTCCCTCTATGCGCATTCGCTCGCTCGGTGGGGCCTGCGG |
| | Site: Nb.Bsml; Format: tl | ACCAAAGGTCCGCAGACGGCAGAGCTCTGCTCTGCCGGCCCCACCGA |
| | | GCGAGCGAATGCGCATAGAGGGAGTGGCCAA |

TABLE 6
Exemplary AAV derived ITRs harboring antiparallel recognition sites for nicking
endonuclease Nb.BsrDI

| SEQ ID NO | Name | Full Sequence |
|---|---|---|
| SEQ ID NO: 62 | source: AAV1; Recogn. | TTGCCCACTCCCGCAATGCGCGCTCGCTCGCTCGGTGGGGCCTGCG |
| | Site: Nb.BsrDI; Format: | GACCAAAGGTCCGCAGACGGCAGAGCTCTGCTCTGCCGGCCCCACC |
| | bl | GAGCGAGCGAGCGCGCATTGCGGGAGTGGGCAA |
| SEQ ID NO: 63 | source: AAV1; Recogn. | TTGCCCACTCCCTCATTGCGCGCTCGCTCGCTCGGTGGGGCCTGCG |
| | Site: Nb.BsrDI; Format: tl | GACCAAAGGTCCGCAGACGGCAGAGCTCTGCTCTGCCGGCCCCACC |
| | | GAGCGAGCGAGCGCGCAATGAGGGAGTGGGCAA |
| SEQ ID NO: 64 | source: AAV2; Recogn. | TTGGCCACTCCCGCAATGCGCGCTCGCTCGCTCACTGAGGCCGGGC |
| | Site: Nb.BsrDI; Format: | GACCAAAGGTCGCCCGACGCCCGGGCTTTGCCCGGGCGGCCTCAGT |
| | bl | GAGCGAGCGAGCGCGCATTGCGGGAGTGGCCAA |
| SEQ ID NO: 65 | source: AAV2; Recogn. | TTGGCCACTCCCTCATTGCGCGCTCGCTCGCTCACTGAGGCCGGGC |
| | Site: Nb.BsrDI; Format: tl | GACCAAAGGTCGCCCGACGCCCGGGCTTTGCCCGGGCGGCCTCAGT |
| | | GAGCGAGCGAGCGCGCAATGAGGGAGTGGCCAA |
| SEQ ID NO: 66 | source: AAV3; Recogn. | TTGGCCACTCCCGCAATGCGCACTCGCTCGCTCGGTGGGGCCTGGC |
| | Site: Nb.BsrDI; Format: | GACCAAAGGTCGCCAGACGGACGTGCTTTGCACGTCCGGCCCCACC |
| | bl | GAGCGAGCGAGTGCGCATTGCGGGAGTGGCCAA |
| SEQ ID NO: 67 | source: AAV3; Recogn. | TTGGCCACTCCCTCATTGCGCACTCGCTCGCTCGGTGGGGCCTGGC |
| | Site: Nb.BsrDI; Format: tl | GACCAAAGGTCGCCAGACGGACGTGCTTTGCACGTCCGGCCCCACC |
| | | GAGCGAGCGAGTGCGCAATGAGGGAGTGGCCAA |
| SEQ ID NO: 68 | source: AAV4 left; | TTGGCCACTCCCGCAATGCGCGCTCGCTCACTCACTCGGCCCTGGA |
| | Recogn. Site: Nb.BsrDI; | GACCAAAGGTCTCCAGACTGCCGGCCTCTGGCCGGCAGGGCCGAGT |
| | Format: bl | GAGTGAGCGAGCGCGCATTGCGGGAGTGGCCAA |
| SEQ ID NO: 69 | source: AAV4 left; | TTGGCCACTCCCTCATTGCGCGCTCGCTCACTCACTCGGCCCTGGA |
| | Recogn. Site: Nb.BsrDI; | GACCAAAGGTCTCCAGACTGCCGGCCTCTGGCCGGCAGGGCCGAGT |
| | Format: tl | GAGTGAGCGAGCGCGCAATGAGGGAGTGGCCAA |
| SEQ ID NO: 70 | source: AAV4 right; | TTGGCCACATTAGCAATGCGCGCTCGCTCACTCACTCGGCCCTGGA |
| | Recogn. Site: Nb.BsrDI; | GACCAAAGGTCTCCAGACTGCCGGCCTCTGGCCGGCAGGGCCGAGT |
| | Format: bl | GAGTGAGCGAGCGCGCATTGCGGGAGTGGCCAA |
| SEQ ID NO: 71 | source: AAV4 right; | TTGGCCACATTAGCATTGCGCGCTCGCTCACTCACTCGGCCCTGGA |
| | Recogn. Site: Nb.BsrDI; | GACCAAAGGTCTCCAGACTGCCGGCCTCTGGCCGGCAGGGCCGAGT |
| | Format: tl | GAGTGAGCGAGCGCGCAATGAGGGAGTGGCCAA |
| SEQ ID NO: 72 | source: AAV5; Recogn. | CTCTCCGCAATGTCGCGTTCGCTCGCTCGCTGGCTCGTTTGGGGGG |
| | Site: Nb.BsrDI; Format: | GTGGCAGCTCAAAGAGCTGCCAGACGACGGCCCTCTGGCCGTCGCC |
| | bl | CCCCCAAACGAGCCAGCGAGCGAGCGAACGCGACATTGCGGAGAG |
| SEQ ID NO: 73 | source: AAV5; Recogn. | CTCTCCCCCATTGCGCGTTCGCTCGCTCGCTGGCTCGTTTGGGGGG |
| | Site: Nb.BsrDI; Format: tl | GTGGCAGCTCAAAGAGCTGCCAGACGACGGCCCTCTGGCCGTCGCC |
| | | CCCCCAAACGAGCCAGCGAGCGAGCGAACGCGCAATGGGGGAGAG |
| SEQ ID NO: 74 | source: AAV7; Recogn. | TTGGCCACTCCCGCAATGCGCGCTCGCTCGCTCGGTGGGGCCTGCG |
| | Site: Nb.BsrDI; Format: | GACCAAAGGTCCGCAGACGGCAGAGCTCTGCTCTGCCGGCCCCACC |
| | bl | GAGCGAGCGAGCGCGCATTGCGGGAGTGGCCAA |
| SEQ ID NO: 75 | source: AAV7; Recogn. | TTGGCCACTCCCTCATTGCGCGCTCGCTCGCTCGGTGGGGCCTGCG |
| | Site: Nb.BsrDI; Format: tl | GACCAAAGGTCCGCAGACGGCAGAGCTCTGCTCTGCCGGCCCCACC |
| | | GAGCGAGCGAGCGCGCAATGAGGGAGTGGCCAA |

TABLE 7
Exemplary AAV derived ITRs harboring antiparallel recognition sites for nicking
endonuclease Nb.BssSi

| SEQ ID NO: | Name | Full Sequence |
|---|---|---|
| SEQ ID NO: 76 | source: AAV1; Recogn. | TTGCCCACGAGCTCTCTGCGCGCTCGCTCGCTCGGTGGGGCCTGCGG |
| | Site: Nb.BssSI; Format: | ACCAAAGGTCCGCAGACGGCAGAGCTCTGCTCTGCCGGCCCCACCGA |
| | bl | GCGAGCGAGCGCGCAGAGAGCTCGTGGGCAA |
| SEQ ID NO: 77 | source: AAV1; Recogn. | TTGCCCACTCCCTCGTGGCGCGCTCGCTCGCTCGGTGGGGCCTGCGG |
| | Site: Nb.BssSI; Format: tl | ACCAAAGGTCCGCAGACGGCAGAGCTCTGCTCTGCCGGCCCCACCGA |
| | | GCGAGCGAGCGCGCCACGAGGGAGTGGGCAA |
| SEQ ID NO: 78 | source: AAV2; Recogn. | TTGGCCACGAGCTCTCTGCGCGCTCGCTCGCTCACTGAGGCCGGGCG |
| | Site: Nb.BssSI; Format: | ACCAAAGGTCGCCCGACGCCCGGGCTTTGCCCGGGCGGCCTCAGTGA |
| | bl | GCGAGCGAGCGCGCAGAGAGCTCGTGGCCAA |
| SEQ ID NO: 79 | source: AAV2; Recogn. | TTGGCCACTCCCTCGTGGCGCGCTCGCTCGCTCACTGAGGCCGGGCG |
| | Site: Nb.BssSI; Format: tl | ACCAAAGGTCGCCCGACGCCCGGGCTTTGCCCGGGCGGCCTCAGTGA |
| | | GCGAGCGAGCGCGCCACGAGGGAGTGGCCAA |
| SEQ ID NO: 80 | source: AAV3; Recogn. | TTGGCCACGAGCTCTATGCGCACTCGCTCGCTCGGTGGGGCCTGGCG |
| | Site: Nb.BssSI; Format: | ACCAAAGGTCGCCAGACGGACGTGCTTTGCACGTCCGGCCCCACCGA |
| | bl | GCGAGCGAGTGCGCATAGAGCTCGTGGCCAA |
| SEQ ID NO: 81 | source: AAV3; Recogn. | TTGGCCACTCCCTCGTGGCGCACTCGCTCGCTCGGTGGGGCCTGGCG |
| | Site: Nb.BssSI; Format: tl | ACCAAAGGTCGCCAGACGGACGTGCTTTGCACGTCCGGCCCCACCGA |
| | | GCGAGCGAGTGCGCCACGAGGGAGTGGCCAA |
| SEQ ID NO: 82 | source: AAV4 left; | TTGGCCACGAGCTCTATGCGCGCTCGCTCACTCACTCGGCCCTGGAG |
| | Recogn. Site: Nb.BssSI; | ACCAAAGGTCTCCAGACTGCCGGCCTCTGGCCGGCAGGGCCGAGTGA |
| | Format: bl | GTGAGCGAGCGCGCATAGAGCTCGTGGCCAA |
| SEQ ID NO: 83 | source: AAV4 left; | TTGGCCACTCCCTCGTGGCGCGCTCGCTCACTCACTCGGCCCTGGAG |
| | Recogn. Site: Nb.BssSI; | ACCAAAGGTCTCCAGACTGCCGGCCTCTGGCCGGCAGGGCCGAGTGA |
| | Format: tl | GTGAGCGAGCGCGCCACGAGGGAGTGGCCAA |
| SEQ ID NO: 84 | source: AAV4 right; | TTGGCCACGAGAGCTATGCGCGCTCGCTCACTCACTCGGCCCTGGAG |
| | Recogn. Site: Nb.BssSI; | ACCAAAGGTCTCCAGACTGCCGGCCTCTGGCCGGCAGGGCCGAGTGA |
| | Format: bl | GTGAGCGAGCGCGCATAGAGCTCGTGGCCAA |
| SEQ ID NO: 85 | source: AAV4 right; | TTGGCCACATTCTCGTGGCGCGCTCGCTCACTCACTCGGCCCTGGAG |
| | Recogn. Site: Nb.BssSI; | ACCAAAGGTCTCCAGACTGCCGGCCTCTGGCCGGCAGGGCCGAGTGA |
| | Format: tl | GTGAGCGAGCGCGCCACGAGGGAGTGGCCAA |
| SEQ ID NO: 86 | source: AAV5; Recogn. | CTCACGAGCCTGTCGCGTTCGCTCGCTCGCTGGCTCGTTTGGGGGGG |
| | Site: Nb.BssSI; Format: | TGGCAGCTCAAAGAGCTGCCAGACGACGGCCCTCTGGCCGTCGCCCC |
| | bl | CCCAAACGAGCCAGCGAGCGAGCGAACGCGACAGGCTCGTGAG |
| SEQ ID NO: 87 | source: AAV5; Recogn. | CTCTCCCTCGTGTCGCGTTCGCTCGCTCGCTGGCTCGTTTGGGGGGG |
| | Site: Nb.BssSI; Format: tl | TGGCAGCTCAAAGAGCTGCCAGACGACGGCCCTCTGGCCGTCGCCCC |
| | | CCCAAACGAGCCAGCGAGCGAGCGAACGCGACACGAGGGAGAG |
| SEQ ID NO: 88 | source: AAV7; Recogn. | TTGGCCACGAGCTCTATGCGCGCTCGCTCGCTCGGTGGGGCCTGCGG |
| | Site: Nb.BssSI; Format: | ACCAAAGGTCCGCAGACGGCAGAGCTCTGCTCTGCCGGCCCCACCGA |
| | bl | GCGAGCGAGCGCGCATAGAGCTCGTGGCCAA |
| SEQ ID NO: 89 | source: AAV7; Recogn. | TTGGCCACTCCCTCGTGGCGCGCTCGCTCGCTCGGTGGGGCCTGCGG |
| | Site: Nb.BssSI; Format: tl | ACCAAAGGTCCGCAGACGGCAGAGCTCTGCTCTGCCGGCCCCACCGA |
| | | GCGAGCGAGCGCGCCACGAGGGAGTGGCCAA |

TABLE 8
Exemplary AAV derived ITRs harboring antiparallel recognition sites for nicking
endonuclease Nb.BtsI:

| SEQ ID NO: | Name | Full Sequence |
|---|---|---|
| SEQ ID NO: 90 | source: AAV1; Recogn. | TTGCCCACTCCCGCAGTGCGCGCTCGCTCGCTCGGTGGGGCCTGCGG |
| | Site: Nb.BtsI; Format: bl | ACCAAAGGTCCGCAGACGGCAGAGCTCTGCTCTGCCGGCCCCACCGA |
| | | GCGAGCGAGCGCGCACTGCGGGAGTGGGCAA |
| SEQ ID NO: 91 | source: AAV1; Recogn. | TTGCCCACTCCCTCACTGCGCGCTCGCTCGCTCGGTGGGGCCTGCGG |
| | Site: Nb.BtsI; Format: tl | ACCAAAGGTCCGCAGACGGCAGAGCTCTGCTCTGCCGGCCCCACCGA |
| | | GCGAGCGAGCGCGCAGTGAGGGAGTGGGCAA |
| SEQ ID NO: 92 | source: AAV2; Recogn. | TTGGCCACTCCCGCAGTGCGCGCTCGCTCGCTCACTGAGGCCGGGCG |
| | Site: Nb.BtsI; Format: bl | ACCAAAGGTCGCCCGACGCCCGGGCTTTGCCCGGGCGGCCTCAGTGA |
| | | GCGAGCGAGCGCGCACTGCGGGAGTGGCCAA |
| SEQ ID NO: 93 | source: AAV2; Recogn. | TTGGCCACTCCCTCACTGCGCGCTCGCTCGCTCACTGAGGCCGGGCG |
| | Site: Nb.BtsI; Format: tl | ACCAAAGGTCGCCCGACGCCCGGGCTTTGCCCGGGCGGCCTCAGTGA |
| | | GCGAGCGAGCGCGCAGTGAGGGAGTGGCCAA |
| SEQ ID NO: 94 | source: AAV3; Recogn. | TTGGCCACTCCCGCAGTGCGCACTCGCTCGCTCGGTGGGGCCTGGCG |
| | Site: Nb.BtsI; Format: bl | ACCAAAGGTCGCCAGACGGACGTGCTTTGCACGTCCGGCCCCACCGA |
| | | GCGAGCGAGTGCGCACTGCGGGAGTGGCCAA |
| SEQ ID NO: 95 | source: AAV3; Recogn. | TTGGCCACTCCCTCACTGCGCACTCGCTCGCTCGGTGGGGCCTGGCG |
| | Site: Nb.BtsI; Format: tl | ACCAAAGGTCGCCAGACGGACGTGCTTTGCACGTCCGGCCCCACCGA |
| | | GCGAGCGAGTGCGCAGTGAGGGAGTGGCCAA |
| SEQ ID NO: 96 | source: AAV4 left; | TTGGCCACTCCCGCAGTGCGCGCTCGCTCACTCACTCGGCCCTGGAG |
| | Recogn. Site: Nb.BtsI; | ACCAAAGGTCTCCAGACTGCCGGCCTCTGGCCGGCAGGGCCGAGTGA |
| | Format: bl | GTGAGCGAGCGCGCACTGCGGGAGTGGCCAA |
| SEQ ID NO: 97 | source: AAV4 left; | TTGGCCACTCCCTCACTGCGCGCTCGCTCACTCACTCGGCCCTGGAG |
| | Recogn. Site: Nb.BtsI; | ACCAAAGGTCTCCAGACTGCCGGCCTCTGGCCGGCAGGGCCGAGTGA |
| | Format: tl | GTGAGCGAGCGCGCAGTGAGGGAGTGGCCAA |
| SEQ ID NO: 98 | source: AAV4 right; | TTGGCCACATTAGCAGTGCGCGCTCGCTCACTCACTCGGCCCTGGAG |
| | Recogn. Site: Nb.BtsI; | ACCAAAGGTCTCCAGACTGCCGGCCTCTGGCCGGCAGGGCCGAGTGA |
| | Format: bl | GTGAGCGAGCGCGCACTGCGGGAGTGGCCAA |
| SEQ ID NO: 99 | source: AAV4 right; | TTGGCCACATTAGCACTGCGCGCTCGCTCACTCACTCGGCCCTGGAG |
| | Recogn. Site: Nb.BtsI; | ACCAAAGGTCTCCAGACTGCCGGCCTCTGGCCGGCAGGGCCGAGTGA |
| | Format: tl | GTGAGCGAGCGCGCAGTGAGGGAGTGGCCAA |
| SEQ ID | source: AAV5; Recogn. | CTCTCCGCAGTGTCGCGTTCGCTCGCTCGCTGGCTCGTTTGGGGGGG |
| NO: 100 | Site: Nb.BtsI; Format: bl | TGGCAGCTCAAAGAGCTGCCAGACGACGGCCCTCTGGCCGTCGCCCC |
| | | CCCAAACGAGCCAGCGAGCGAGCGAACGCGACACTGCGGAGAG |
| SEQ ID | source: AAV5; Recogn. | CTCTCCCCACTGCCGCGTTCGCTCGCTCGCTGGCTCGTTTGGGGGGG |
| NO: 101 | Site: Nb.BtsI; Format: tl | TGGCAGCTCAAAGAGCTGCCAGACGACGGCCCTCTGGCCGTCGCCCC |
| | | CCCAAACGAGCCAGCGAGCGAGCGAACGCGGCAGTGGGGAGAG |
| SEQ ID | source: AAV7; Recogn. | TTGGCCACTCCCGCAGTGCGCGCTCGCTCGCTCGGTGGGGCCTGCGG |
| NO: 102 | Site: Nb.BtsI; Format: bl | ACCAAAGGTCCGCAGACGGCAGAGCTCTGCTCTGCCGGCCCCACCGA |
| | | GCGAGCGAGCGCGCACTGCGGGAGTGGCCAA |
| SEQ ID | source: AAV7; Recogn. | TTGGCCACTCCCTCACTGCGCGCTCGCTCGCTCGGTGGGGCCTGCGG |
| NO: 103 | Site: Nb.BtsI; Format: tl | ACCAAAGGTCCGCAGACGGCAGAGCTCTGCTCTGCCGGCCCCACCGA |
| | | GCGAGCGAGCGCGCAGTGAGGGAGTGGCCAA |

TABLE 9
Exemplary AAV derived ITRs harboring antiparallel recognition sites for nicking
endonuclease Nt.AlwI:

| SEQ ID NO: | Name | Full Sequence |
|---|---|---|
| SEQ ID | source: AAV1; Recogn. | TTGCCCACTCCCTCGATCCGCGCTCGCTCGCTCGGTGGGGCCTGCGG |
| NO: 104 | Site: Nt.AlwI; Format: bl | ACCAAAGGTCCGCAGACGGCAGAGCTCTGCTCTGCCGGCCCCACCGA |
| | | GCGAGCGAGCGCGGATCGAGGGAGTGGGCAA |
| SEQ ID | source: AAV1; Recogn. | TTGCCCACTGGATCTCTGCGCGCTCGCTCGCTCGGTGGGGCCTGCGG |
| NO: 105 | Site: Nt.AlwI; Format: tl | ACCAAAGGTCCGCAGACGGCAGAGCTCTGCTCTGCCGGCCCCACCGA |
| | | GCGAGCGAGCGCGCAGAGATCCAGTGGGCAA |
| SEQ ID | source: AAV2; Recogn. | TTGGCCACTCCCTCGATCCGCGCTCGCTCGCTCACTGAGGCCGGGCG |
| NO: 106 | Site: Nt.AlwI; Format: bl | ACCAAAGGTCGCCCGACGCCCGGGCTTTGCCCGGGCGGCCTCAGTGA |
| | | GCGAGCGAGCGCGGATCGAGGGAGTGGCCAA |
| SEQ ID | source: AAV2; Recogn. | TTGGCCACTGGATCTCTGCGCGCTCGCTCGCTCACTGAGGCCGGGCG |
| NO: 107 | Site: Nt.AlwI; Format: tl | ACCAAAGGTCGCCCGACGCCCGGGCTTTGCCCGGGCGGCCTCAGTGA |
| | | GCGAGCGAGCGCGCAGAGATCCAGTGGCCAA |
| SEQ ID | source: AAV3; Recogn. | TTGGCCACTCCCTCGATCCGCACTCGCTCGCTCGGTGGGGCCTGGCG |
| NO: 108 | Site: Nt.AlwI; Format: bl | ACCAAAGGTCGCCAGACGGACGTGCTTTGCACGTCCGGCCCCACCGA |
| | | GCGAGCGAGTGCGGATCGAGGGAGTGGCCAA |
| SEQ ID | source: AAV3; Recogn. | TTGGCCACTGGATCTATGCGCACTCGCTCGCTCGGTGGGGCCTGGCG |
| NO: 109 | Site: Nt.AlwI; Format: tl | ACCAAAGGTCGCCAGACGGACGTGCTTTGCACGTCCGGCCCCACCGA |
| | | GCGAGCGAGTGCGCATAGATCCAGTGGCCAA |
| SEQ ID | source: AAV4 left; | TTGGCCACTCCCTCGATCCGCGCTCGCTCACTCACTCGGCCCTGGAG |
| NO: 110 | Recogn. Site: Nt.AlwI; | ACCAAAGGTCTCCAGACTGCCGGCCTCTGGCCGGCAGGGCCGAGTGA |
| | Format: bl | GTGAGCGAGCGCGGATCGAGGGAGTGGCCAA |
| SEQ ID | source: AAV4 left; | TTGGCCACTGGATCTATGCGCGCTCGCTCACTCACTCGGCCCTGGAG |
| NO: 111 | Recogn. Site: Nt.AlwI; | ACCAAAGGTCTCCAGACTGCCGGCCTCTGGCCGGCAGGGCCGAGTGA |
| | Format: tl | GTGAGCGAGCGCGCATAGATCCAGTGGCCAA |
| SEQ ID | source: AAV4 right; | TTGGCCACATTAGCGATCCGCGCTCGCTCACTCACTCGGCCCTGGAG |
| NO: 112 | Recogn. Site: Nt.AlwI; | ACCAAAGGTCTCCAGACTGCCGGCCTCTGGCCGGCAGGGCCGAGTGA |
| | Format: bl | GTGAGCGAGCGCGGATCGAGGGAGTGGCCAA |
| SEQ ID | source: AAV4 right; | TTGGCCACAGGATCTATGCGCGCTCGCTCACTCACTCGGCCCTGGAG |
| NO: 113 | Recogn. Site: Nt.AlwI; | ACCAAAGGTCTCCAGACTGCCGGCCTCTGGCCGGCAGGGCCGAGTGA |
| | Format: tl | GTGAGCGAGCGCGCATAGATCCAGTGGCCAA |
| SEQ ID | source: AAV5; Recogn. | CTCTCCCCCCTGTCGCGATCCCTCGCTCGCTGGCTCGTTTGGGGGGG |
| NO: 114 | Site: Nt.AlwI; Format: bl | TGGCAGCTCAAAGAGCTGCCAGACGACGGCCCTCTGGCCGTCGCCCC |
| | | CCCAAACGAGCCAGCGAGCGAGGGATCGCGACAGGGGGGAGAG |
| SEQ ID | source: AAV5; Recogn. | CTCTCCCCCGGATCGCGTTCGCTCGCTCGCTGGCTCGTTTGGGGGGG |
| NO: 115 | Site: Nt.AlwI; Format: tl | TGGCAGCTCAAAGAGCTGCCAGACGACGGCCCTCTGGCCGTCGCCCC |
| | | CCCAAACGAGCCAGCGAGCGAGCGAACGCGATCCGGGGGAGAG |
| SEQ ID | source: AAV7; Recogn. | TTGGCCACTCCCTCGATCCGCGCTCGCTCGCTCGGTGGGGCCTGCGG |
| NO: 116 | Site: Nt.AlwI; Format: bl | ACCAAAGGTCCGCAGACGGCAGAGCTCTGCTCTGCCGGCCCCACCGA |
| | | GCGAGCGAGCGCGGATCGAGGGAGTGGCCAA |
| SEQ ID | source: AAV7; Recogn. | TTGGCCACTGGATCTATGCGCGCTCGCTCGCTCGGTGGGGCCTGCGG |
| NO: 117 | Site: Nt.AlwI; Format: tl | ACCAAAGGTCCGCAGACGGCAGAGCTCTGCTCTGCCGGCCCCACCGA |
| | | GCGAGCGAGCGCGCATAGATCCAGTGGCCAA |

TABLE 10
Exemplary AAV derived ITRs harboring antiparallel recognition sites for nicking
endonuclease Nt.BbvCI:

| SEQ ID NO: | Name | Full Sequence |
|---|---|---|
| SEQ ID | source: AAV1; Recogn. | TTGCCCACTCCCGCTGAGGGCGCTCGCTCGCTCGGTGGGGCCTGCGGA |
| NO: 118 | Site: Nt.BbvCI; Format: | CCAAAGGTCCGCAGACGGCAGAGCTCTGCTCTGCCGGCCCCACCGAGC |
| | bl | GAGCGAGCGCCCTCAGCGGGAGTGGGCAA |
| SEQ ID | source: AAV1; Recogn. | TTGCCCACTCCCCCTCAGCGCGCTCGCTCGCTCGGTGGGGCCTGCGGA |
| NO: 119 | Site: Nt.BbvCI; Format: tl | CCAAAGGTCCGCAGACGGCAGAGCTCTGCTCTGCCGGCCCCACCGAGC |
| | | GAGCGAGCGCGCTGAGGGGGAGTGGGCAA |
| SEQ ID | source: AAV2; Recogn. | TTGGCCACTCCCGCTGAGGGCGCTCGCTCGCTCACTGAGGCCGGGCGA |
| NO: 120 | Site: Nt.BbvCI; Format: | CCAAAGGTCGCCCGACGCCCGGGCTTTGCCCGGGCGGCCTCAGTGAGC |
| | bl | GAGCGAGCGCCCTCAGCGGGAGTGGCCAA |
| SEQ ID | source: AAV2; Recogn. | TTGGCCACTCCCCCTCAGCGCGCTCGCTCGCTCACTGAGGCCGGGCGA |
| NO: 121 | Site: Nt.BbvCI; Format: tl | CCAAAGGTCGCCCGACGCCCGGGCTTTGCCCGGGCGGCCTCAGTGAGC |
| | | GAGCGAGCGCGCTGAGGGGGAGTGGCCAA |
| SEQ ID | source: AAV3; Recogn. | TTGGCCACTCCCGCTGAGGGCACTCGCTCGCTCGGTGGGGCCTGGCGA |
| NO: 122 | Site: Nt.BbvCI; Format: | CCAAAGGTCGCCAGACGGACGTGCTTTGCACGTCCGGCCCCACCGAGC |
| | bl | GAGCGAGTGCCCTCAGCGGGAGTGGCCAA |
| SEQ ID | source: AAV3; Recogn. | TTGGCCACTCCCCCTCAGCGCACTCGCTCGCTCGGTGGGGCCTGGCGA |
| NO: 123 | Site: Nt.BbvCI; Format: tl | CCAAAGGTCGCCAGACGGACGTGCTTTGCACGTCCGGCCCCACCGAGC |
| | | GAGCGAGTGCGCTGAGGGGGAGTGGCCAA |
| SEQ ID | source: AAV4 left; | TTGGCCACTCCCGCTGAGGGCGCTCGCTCACTCACTCGGCCCTGGAGA |
| NO: 124 | Recogn. Site: Nt.BbvCI; | CCAAAGGTCTCCAGACTGCCGGCCTCTGGCCGGCAGGGCCGAGTGAGT |
| | Format: bl | GAGCGAGCGCCCTCAGCGGGAGTGGCCAA |
| SEQ ID | source: AAV4 left; | TTGGCCACTCCCCCTCAGCGCGCTCGCTCACTCACTCGGCCCTGGAGA |
| NO: 125 | Recogn. Site: Nt.BbvCI; | CCAAAGGTCTCCAGACTGCCGGCCTCTGGCCGGCAGGGCCGAGTGAGT |
| | Format: tl | GAGCGAGCGCGCTGAGGGGGAGTGGCCAA |
| SEQ ID | source: AAV4 right; | TTGGCCACATTAGCTGAGGGCGCTCGCTCACTCACTCGGCCCTGGAGA |
| NO: 126 | Recogn. Site: Nt.BbvCI; | CCAAAGGTCTCCAGACTGCCGGCCTCTGGCCGGCAGGGCCGAGTGAGT |
| | Format: bl | GAGCGAGCGCCCTCAGCGGGAGTGGCCAA |
| SEQ ID | source: AAV4 right; | TTGGCCACATTACCTCAGCGCGCTCGCTCACTCACTCGGCCCTGGAGA |
| NO: 127 | Recogn. Site: Nt.BbvCI; | CCAAAGGTCTCCAGACTGCCGGCCTCTGGCCGGCAGGGCCGAGTGAGT |
| | Format: tl | GAGCGAGCGCGCTGAGGGGGAGTGGCCAA |
| SEQ ID | source: AAV5; Recogn. | CTCTCCCCGCTGAGGCGTTCGCTCGCTCGCTGGCTCGTTTGGGGGGGT |
| NO: 128 | Site: Nt.BbvCI; Format: | GGCAGCTCAAAGAGCTGCCAGACGACGGCCCTCTGGCCGTCGCCCCCC |
| | bl | CAAACGAGCCAGCGAGCGAGCGAACGCCTCAGCGGGGAGAG |
| SEQ ID | source: AAV5; Recogn. | CTCTCCCCTCAGCCGCGTTCGCTCGCTCGCTGGCTCGTTTGGGGGGGT |
| NO: 129 | Site: Nt.BbvCI; Format: tl | GGCAGCTCAAAGAGCTGCCAGACGACGGCCCTCTGGCCGTCGCCCCCC |
| | | CAAACGAGCCAGCGAGCGAGCGAACGCGGCTGAGGGGAGAG |
| SEQ ID | source: AAV7; Recogn. | TTGGCCACTCCCGCTGAGGGCGCTCGCTCGCTCGGTGGGGCCTGCGGA |
| NO: 130 | Site: Nt.BbvCI; Format: | CCAAAGGTCCGCAGACGGCAGAGCTCTGCTCTGCCGGCCCCACCGAGC |
| | bl | GAGCGAGCGCCCTCAGCGGGAGTGGCCAA |
| SEQ ID | source: AAV7; Recogn. | TTGGCCACTCCCCCTCAGCGCGCTCGCTCGCTCGGTGGGGCCTGCGGA |
| NO: 131 | Site: Nt.BbvCI; Format: tl | CCAAAGGTCCGCAGACGGCAGAGCTCTGCTCTGCCGGCCCCACCGAGC |
| | | GAGCGAGCGCGCTGAGGGGGAGTGGCCAA |

TABLE 11
Exemplary AAV derived ITRs harboring antiparallel recognition sites for nicking
endonuclease Nt.BsmAI:

| SEQ ID NO: | Name | Full Sequence |
|---|---|---|
| SEQ ID | source: AAV1; Recogn. | TTGCCCACTGAGACTCTGCGCGCTCGCTCGCTCGGTGGGGCCTGCGGA |
| NO: 132 | Site: Nt.BsmAI; Format: | CCAAAGGTCCGCAGACGGCAGAGCTCTGCTCTGCCGGCCCCACCGAGC |
| | bl | GAGCGAGCGCGCAGAGTCTCAGTGGGCAA |
| SEQ ID | source: AAV1; Recogn. | TTGCCCACTCCGTCTCTGCGCGCTCGCTCGCTCGGTGGGGCCTGCGGA |
| NO: 133 | Site: Nt.BsmAI; Format: | CCAAAGGTCCGCAGACGGCAGAGCTCTGCTCTGCCGGCCCCACCGAGC |
| | t1 | GAGCGAGCGCGCAGAGACGGAGTGGGCAA |
| SEQ ID | source: AAV2; Recogn. | TTGGCCACTGAGACTCTGCGCGCTCGCTCGCTCACTGAGGCCGGGCGA |
| NO: 134 | Site: Nt.BsmAI; Format: | CCAAAGGTCGCCCGACGCCCGGGCTTTGCCCGGGCGGCCTCAGTGAGC |
| | bl | GAGCGAGCGCGCAGAGTCTCAGTGGCCAA |
| SEQ ID | source: AAV2; Recogn. | TTGGCCACTCCGTCTCTGCGCGCTCGCTCGCTCACTGAGGCCGGGCGA |
| NO: 135 | Site: Nt.BsmAI; Format: | CCAAAGGTCGCCCGACGCCCGGGCTTTGCCCGGGCGGCCTCAGTGAGC |
| | tl | GAGCGAGCGCGCAGAGACGGAGTGGCCAA |
| SEQ ID | source: AAV3; Recogn. | TTGGCCACTGAGACTATGCGCACTCGCTCGCTCGGTGGGGCCTGGCGA |
| NO: 136 | Site: Nt.BsmAI; Format: | CCAAAGGTCGCCAGACGGACGTGCTTTGCACGTCCGGCCCCACCGAGC |
| | bl | GAGCGAGTGCGCATAGTCTCAGTGGCCAA |
| SEQ ID | source: AAV3; Recogn. | TTGGCCACTCCGTCTCTGCGCACTCGCTCGCTCGGTGGGGCCTGGCGA |
| NO: 137 | Site: Nt.BsmAI; Format: | CCAAAGGTCGCCAGACGGACGTGCTTTGCACGTCCGGCCCCACCGAGC |
| | tl | GAGCGAGTGCGCAGAGACGGAGTGGCCAA |
| SEQ ID | source: AAV4 left; | TTGGCCACTGAGACTATGCGCGCTCGCTCACTCACTCGGCCCTGGAGA |
| NO: 138 | Recogn. Site: Nt.BsmAI; | CCAAAGGTCTCCAGACTGCCGGCCTCTGGCCGGCAGGGCCGAGTGAGT |
| | Format: bl | GAGCGAGCGCGCATAGTCTCAGTGGCCAA |
| SEQ ID | source: AAV4 left; | TTGGCCACTCCGTCTCTGCGCGCTCGCTCACTCACTCGGCCCTGGAGA |
| NO: 139 | Recogn. Site: Nt.BsmAI; | CCAAAGGTCTCCAGACTGCCGGCCTCTGGCCGGCAGGGCCGAGTGAGT |
| | Format: tl | GAGCGAGCGCGCAGAGACGGAGTGGCCAA |
| SEQ ID | source: AAV4 right; | TTGGCCACAGAGACTATGCGCGCTCGCTCACTCACTCGGCCCTGGAGA |
| NO: 140 | Recogn. Site: Nt.BsmAI; | CCAAAGGTCTCCAGACTGCCGGCCTCTGGCCGGCAGGGCCGAGTGAGT |
| | Format: bl | GAGCGAGCGCGCATAGTCTCAGTGGCCAA |
| SEQ ID | source: AAV4 right; | TTGGCCACATTGTCTCTGCGCGCTCGCTCACTCACTCGGCCCTGGAGA |
| NO: 141 | Recogn. Site: Nt.BsmAI; | CCAAAGGTCTCCAGACTGCCGGCCTCTGGCCGGCAGGGCCGAGTGAGT |
| | Format: tl | GAGCGAGCGCGCAGAGACGGAGTGGCCAA |
| SEQ ID | source: AAV5; Recogn. | CTCTCCCCCGAGACGCGTTCGCTCGCTCGCTGGCTCGTTTGGGGGGGT |
| NO: 142 | Site: Nt.BsmAI; Format: | GGCAGCTCAAAGAGCTGCCAGACGACGGCCCTCTGGCCGTCGCCCCCC |
| | bl | CAAACGAGCCAGCGAGCGAGCGAACGCGTCTCGGGGGAGAG |
| SEQ ID | source: AAV5; Recogn. | CTCTCCCCCGTCTCGCGTTCGCTCGCTCGCTGGCTCGTTTGGGGGGGT |
| NO: 143 | Site: Nt.BsmAI; Format: | GGCAGCTCAAAGAGCTGCCAGACGACGGCCCTCTGGCCGTCGCCCCCC |
| | tl | CAAACGAGCCAGCGAGCGAGCGAACGCGAGACGGGGGAGAG |
| SEQ ID | source: AAV7; Recogn. | TTGGCCACTGAGACTATGCGCGCTCGCTCGCTCGGTGGGGCCTGCGGA |
| NO: 144 | Site: Nt.BsmAI; Format: | CCAAAGGTCCGCAGACGGCAGAGCTCTGCTCTGCCGGCCCCACCGAGC |
| | bl | GAGCGAGCGCGCATAGTCTCAGTGGCCAA |
| SEQ ID | source: AAV7; Recogn. | TTGGCCACTCCGTCTCTGCGCGCTCGCTCGCTCGGTGGGGCCTGCGGA |
| NO: 145 | Site: Nt.BsmAI; Format: | CCAAAGGTCCGCAGACGGCAGAGCTCTGCTCTGCCGGCCCCACCGAGC |
| | tl | GAGCGAGCGCGCAGAGACGGAGTGGCCAA |

TABLE 12
Exemplary AAV derived ITRs harboring antiparallel recognition sites for nicking
endonuclease Nt.BspQI:

| SEQ ID NO: | Name | Full Sequence |
|---|---|---|
| SEQ ID | source: AAV1; Recogn. | TTGCCCACTCCCGAAGAGCGCGCTCGCTCGCTCGGTGGGGCCTGCGGA |
| NO: 146 | Site: Nt.BspQI; Format: | CCAAAGGTCCGCAGACGGCAGAGCTCTGCTCTGCCGGCCCCACCGAGC |
| | bl | GAGCGAGCGCGCTCTTCGGGAGTGGGCAA |
| SEQ ID | source: AAV1; Recogn. | TTGCCCACTCCCGCTCTTCGCGCTCGCTCGCTCGGTGGGGCCTGCGGA |
| NO: 147 | Site: Nt.BspQI; Format: tl | CCAAAGGTCCGCAGACGGCAGAGCTCTGCTCTGCCGGCCCCACCGAGC |
| | | GAGCGAGCGCGAAGAGCGGGAGTGGGCAA |
| SEQ ID | source: AAV2; Recogn. | TTGGCCACTCCCGAAGAGCGCGCTCGCTCGCTCACTGAGGCCGGGCGA |
| NO: 148 | Site: Nt.BspQI; Format: | CCAAAGGTCGCCCGACGCCCGGGCTTTGCCCGGGCGGCCTCAGTGAGC |
| | bl | GAGCGAGCGCGCTCTTCGGGAGTGGCCAA |
| SEQ ID | source: AAV2; Recogn. | TTGGCCACTCCCGCTCTTCGCGCTCGCTCGCTCACTGAGGCCGGGCGA |
| NO: 149 | Site: Nt.BspQI; Format: tl | CCAAAGGTCGCCCGACGCCCGGGCTTTGCCCGGGCGGCCTCAGTGAGC |
| | | GAGCGAGCGCGAAGAGCGGGAGTGGCCAA |
| SEQ ID | source: AAV3; Recogn. | TTGGCCACTCCCGAAGAGCGCACTCGCTCGCTCGGTGGGGCCTGGCGA |
| NO: 150 | Site: Nt.BspQI; Format: | CCAAAGGTCGCCAGACGGACGTGCTTTGCACGTCCGGCCCCACCGAGC |
| | bl | GAGCGAGTGCGCTCTTCGGGAGTGGCCAA |
| SEQ ID | source: AAV3; Recogn. | TTGGCCACTCCCGCTCTTCGCACTCGCTCGCTCGGTGGGGCCTGGCGA |
| NO: 151 | Site: Nt.BspQI; Format: tl | CCAAAGGTCGCCAGACGGACGTGCTTTGCACGTCCGGCCCCACCGAGC |
| | | GAGCGAGTGCGAAGAGCGGGAGTGGCCAA |
| SEQ ID | source: AAV4 left; | TTGGCCACTCCCGAAGAGCGCGCTCGCTCACTCACTCGGCCCTGGAGA |
| NO: 152 | Recogn. Site: Nt.BspQI; | CCAAAGGTCTCCAGACTGCCGGCCTCTGGCCGGCAGGGCCGAGTGAGT |
| | Format: bl | GAGCGAGCGCGCTCTTCGGGAGTGGCCAA |
| SEQ ID | source: AAV4 left; | TTGGCCACTCCCGCTCTTCGCGCTCGCTCACTCACTCGGCCCTGGAGA |
| NO: 153 | Recogn. Site: Nt.BspQI; | CCAAAGGTCTCCAGACTGCCGGCCTCTGGCCGGCAGGGCCGAGTGAGT |
| | Format: tl | GAGCGAGCGCGAAGAGCGGGAGTGGCCAA |
| SEQ ID | source: AAV4 right; | TTGGCCACATTAGAAGAGCGCGCTCGCTCACTCACTCGGCCCTGGAGA |
| NO: 154 | Recogn. Site: Nt.BspQI; | CCAAAGGTCTCCAGACTGCCGGCCTCTGGCCGGCAGGGCCGAGTGAGT |
| | Format: bl | GAGCGAGCGCGCTCTTCGGGAGTGGCCAA |
| SEQ ID | source: AAV4 right; | TTGGCCACATTAGCTCTTCGCGCTCGCTCACTCACTCGGCCCTGGAGA |
| NO: 155 | Recogn. Site: Nt.BspQI; | CCAAAGGTCTCCAGACTGCCGGCCTCTGGCCGGCAGGGCCGAGTGAGT |
| | Format: tl | GAGCGAGCGCGAAGAGCGGGAGTGGCCAA |
| SEQ ID | source: AAV5; Recogn. | CTCTCCCGAAGAGCGCGTTCGCTCGCTCGCTGGCTCGTTTGGGGGGGT |
| NO: 156 | Site: Nt.BspQI; Format: | GGCAGCTCAAAGAGCTGCCAGACGACGGCCCTCTGGCCGTCGCCCCCC |
| | bl | CAAACGAGCCAGCGAGCGAGCGAACGCGCTCTTCGGGAGAG |
| SEQ ID | source: AAV5; Recogn. | CTCTCCCGCTCTTCGCGTTCGCTCGCTCGCTGGCTCGTTTGGGGGGGT |
| NO: 157 | Site: Nt.BspQI; Format: tl | GGCAGCTCAAAGAGCTGCCAGACGACGGCCCTCTGGCCGTCGCCCCCC |
| | | CAAACGAGCCAGCGAGCGAGCGAACGCGAAGAGCGGGAGAG |
| SEQ ID | source: AAV7; Recogn. | TTGGCCACTCCCGAAGAGCGCGCTCGCTCGCTCGGTGGGGCCTGCGGA |
| NO: 158 | Site: Nt.BspQI; Format: | CCAAAGGTCCGCAGACGGCAGAGCTCTGCTCTGCCGGCCCCACCGAGC |
| | bl | GAGCGAGCGCGCTCTTCGGGAGTGGCCAA |
| SEQ ID | source: AAV7; Recogn. | TTGGCCACTCCCGCTCTTCGCGCTCGCTCGCTCGGTGGGGCCTGCGGA |
| NO: 159 | Site: Nt.BspQI; Format: tl | CCAAAGGTCCGCAGACGGCAGAGCTCTGCTCTGCCGGCCCCACCGAGC |
| | | GAGCGAGCGCGAAGAGCGGGAGTGGCCAA |

TABLE 13
Exemplary AAV derived ITRs harboring antiparallel recognition sites for nicking
endonuclease Nt.BstNBI:

| SEQ ID NO: | Name | Full Sequence |
|---|---|---|
| SEQ ID | source: AAV1; Recogn. | TTGCCCACTCCCTCTCTGCGCGACTCGCTCGCTCGGTGGGGCCTGCGG |
| NO: 160 | Site: Nt.BstNBI; Format: | ACCAAAGGTCCGCAGACGGCAGAGCTCTGCTCTGCCGGCCCCACCGAG |
| | bl | CGAGCGAGTCGCGCAGAGAGGGAGTGGGCAA |
| SEQ ID | source: AAV1; Recogn. | TTGCCGAGTCCCTCTCTGCGCGCTCGCTCGCTCGGTGGGGCCTGCGGA |
| NO: 161 | Site: Nt.BstNBI; Format: | CCAAAGGTCCGCAGACGGCAGAGCTCTGCTCTGCCGGCCCCACCGAGC |
| | tl | GAGCGAGCGCGCAGAGAGGGACTCGGCAA |
| SEQ ID | source: AAV2; Recogn. | TTGGCCACTCCCTCTCTGCGCGACTCGCTCGCTCACTGAGGCCGGGCG |
| NO: 162 | Site: Nt.BstNBI; Format: | ACCAAAGGTCGCCCGACGCCCGGGCTTTGCCCGGGCGGCCTCAGTGAG |
| | bl | CGAGCGAGTCGCGCAGAGAGGGAGTGGCCAA |
| SEQ ID | source: AAV2; Recogn. | TTGGCGAGTCCCTCTCTGCGCGCTCGCTCGCTCACTGAGGCCGGGCGA |
| NO: 163 | Site: Nt.BstNBI; Format: | CCAAAGGTCGCCCGACGCCCGGGCTTTGCCCGGGCGGCCTCAGTGAGC |
| | tl | GAGCGAGCGCGCAGAGAGGGACTCGCCAA |
| SEQ ID | source: AAV3; Recogn. | TTGGCCACTCCCTCTATGCGCGACTCGCTCGCTCGGTGGGGCCTGGCG |
| NO: 164 | Site: Nt.BstNBI; Format: | ACCAAAGGTCGCCAGACGGACGTGCTTTGCACGTCCGGCCCCACCGAG |
| | bl | CGAGCGAGTCGCGCATAGAGGGAGTGGCCAA |
| SEQ ID | source: AAV3; Recogn. | TTGGCGAGTCCCTCTATGCGCACTCGCTCGCTCGGTGGGGCCTGGCGA |
| NO: 165 | Site: Nt.BstNBI; Format: | CCAAAGGTCGCCAGACGGACGTGCTTTGCACGTCCGGCCCCACCGAGC |
| | t1 | GAGCGAGTGCGCATAGAGGGACTCGCCAA |
| SEQ ID | source: AAV4 left; | TTGGCCACTCCCTCTATGCGCGACTCGCTCACTCACTCGGCCCTGGAG |
| NO: 166 | Recogn. Site: Nt.BstNBI; | ACCAAAGGTCTCCAGACTGCCGGCCTCTGGCCGGCAGGGCCGAGTGAG |
| | Format: bl | TGAGCGAGTCGCGCATAGAGGGAGTGGCCAA |
| SEQ ID | source: AAV4 left; | TTGGCGAGTCCCTCTATGCGCGCTCGCTCACTCACTCGGCCCTGGAGA |
| NO: 167 | Recogn. Site: Nt.BstNBI; | CCAAAGGTCTCCAGACTGCCGGCCTCTGGCCGGCAGGGCCGAGTGAGT |
| | Format: tl | GAGCGAGCGCGCATAGAGGGACTCGCCAA |
| SEQ ID | source: AAV4 right; | TTGGCCACATTAGCTATGCGCGACTCGCTCACTCACTCGGCCCTGGAG |
| NO: 168 | Recogn. Site: Nt.BstNBI; | ACCAAAGGTCTCCAGACTGCCGGCCTCTGGCCGGCAGGGCCGAGTGAG |
| | Format: bl | TGAGCGAGTCGCGCATAGAGGGAGTGGCCAA |
| SEQ ID | source: AAV4 right; | TTGGCCAGAGTCGCTATGCGCGCTCGCTCACTCACTCGGCCCTGGAGA |
| NO: 169 | Recogn. Site: Nt.BstNBI; | CCAAAGGTCTCCAGACTGCCGGCCTCTGGCCGGCAGGGCCGAGTGAGT |
| | Format: tl | GAGCGAGCGCGCATAGAGACTCTGGCCAA |
| SEQ ID | source: AAV5; Recogn. | CTCTCCCCCCTGTCGCGACTCGCTCGCTCGCTGGCTCGTTTGGGGGGG |
| NO: 170 | Site: Nt.BstNBI; Format: | TGGCAGCTCAAAGAGCTGCCAGACGACGGCCCTCTGGCCGTCGCCCCC |
| | bl | CCAAACGAGCCAGCGAGCGAGCGAGTCGCGACAGGGGGGAGAG |
| SEQ ID | source: AAV5; Recogn. | CTCTCCCCCGAGTCGCGTTCGCTCGCTCGCTGGCTCGTTTGGGGGGGT |
| NO: 171 | Site: Nt.BstNBI; Format: | GGCAGCTCAAAGAGCTGCCAGACGACGGCCCTCTGGCCGTCGCCCCCC |
| | tl | CAAACGAGCCAGCGAGCGAGCGAACGCGACTCGGGGGAGAG |
| SEQ ID | source: AAV7; Recogn. | TTGGCCACTCCCTCTATGCGCGACTCGCTCGCTCGGTGGGGCCTGCGG |
| NO: 172 | Site: Nt.BstNBI; Format: | ACCAAAGGTCCGCAGACGGCAGAGCTCTGCTCTGCCGGCCCCACCGAG |
| | bl | CGAGCGAGTCGCGCATAGAGGGAGTGGCCAA |
| SEQ ID | source: AAV7; Recogn. | TTGGCGAGTCCCTCTATGCGCGCTCGCTCGCTCGGTGGGGCCTGCGGA |
| NO: 173 | Site: Nt.BstNBI; Format: | CCAAAGGTCCGCAGACGGCAGAGCTCTGCTCTGCCGGCCCCACCGAGC |
| | tl | GAGCGAGCGCGCATAGAGGGACTCGCCAA |

TABLE 14
Reverse Complement of Nicking Enzyme Targets

| SEQ ID NO | Name | Sequence |
|---|---|---|
| SEQ ID | wt_AAV1 | AACGGGTGAGGGAGAGACGCGCGAGCGAGCGAGCCACCCCGGACGCCT |
| NO: 174 | | GGTTTCCAGGCGTCTGCCGTCTCGAGACGAGACGGCCGGGGTGGCTCG |
| | | CTC |
| SEQ ID | AAV1_Nb.BbvCI_BL | AACGGGTGAGGGGGAGTCGCGCGAGCGAGCGAGCCACCCCGGACGCCT |
| NO: 175 | | GGTTTCCAGGCGTCTGCCGTCTCGAGACGAGACGGCCGGGGTGGCTCG |
| | | CTC |
| SEQ ID | AAV1_Nb.BbvCI_TL | AACGGGTGAGGGCGACTCCCGCGAGCGAGCGAGCCACCCCGGACGCCT |
| NO: 176 | | GGTTTCCAGGCGTCTGCCGTCTCGAGACGAGACGGCCGGGGTGGCTCG |
| | | CTC |
| SEQ ID | AAV1_Nb.BsmI_BL | AACGGGTGAGGGACTTACGCGCGAGCGAGCGAGCCACCCCGGACGCCT |
| NO: 177 | | GGTTTCCAGGCGTCTGCCGTCTCGAGACGAGACGGCCGGGGTGGCTCG |
| | | CTC |
| SEQ ID | AAV1_Nb.BsmI_TL | AACGGGTGAGGGAGAGACGCGTAAGCGAGCGAGCCACCCCGGACGCCT |
| NO: 178 | | GGTTTCCAGGCGTCTGCCGTCTCGAGACGAGACGGCCGGGGTGGCTCG |
| | | CTC |
| SEQ ID | AAV1_Nb.BsrDI_BL | AACGGGTGAGGGCGTTACGCGCGAGCGAGCGAGCCACCCCGGACGCCT |
| NO: 179 | | GGTTTCCAGGCGTCTGCCGTCTCGAGACGAGACGGCCGGGGTGGCTCG |
| | | CTC |
| SEQ ID | AAV1_Nb.BsrDI_TL | AACGGGTGAGGGAGTAACGCGCGAGCGAGCGAGCCACCCCGGACGCCT |
| NO: 180 | | GGTTTCCAGGCGTCTGCCGTCTCGAGACGAGACGGCCGGGGTGGCTCG |
| | | CTC |
| SEQ ID | AAV1_Nb.BssSI_BL | AACGGGTGCTCGAGAGACGCGCGAGCGAGCGAGCCACCCCGGACGCCT |
| NO: 181 | | GGTTTCCAGGCGTCTGCCGTCTCGAGACGAGACGGCCGGGGTGGCTCG |
| | | CTC |
| SEQ ID | AAV1_Nb.BssSI_TL | AACGGGTGAGGGAGCACCGCGCGAGCGAGCGAGCCACCCCGGACGCCT |
| NO: 182 | | GGTTTCCAGGCGTCTGCCGTCTCGAGACGAGACGGCCGGGGTGGCTCG |
| | | CTC |
| SEQ ID | AAV1_Nb.BtsI_BL | AACGGGTGAGGGCGTCACGCGCGAGCGAGCGAGCCACCCCGGACGCCT |
| NO: 183 | | GGTTTCCAGGCGTCTGCCGTCTCGAGACGAGACGGCCGGGGTGGCTCG |
| | | CTC |
| SEQ ID | AAV1_Nb.BtsI_TL | AACGGGTGAGGGAGTGACGCGCGAGCGAGCGAGCCACCCCGGACGCCT |
| NO: 184 | | GGTTTCCAGGCGTCTGCCGTCTCGAGACGAGACGGCCGGGGTGGCTCG |
| | | CTC |
| SEQ ID | AAV1_Nt.AlwI_BL | AACGGGTGAGGGAGCTAGGCGCGAGCGAGCGAGCCACCCCGGACGCCT |
| NO: 185 | | GGTTTCCAGGCGTCTGCCGTCTCGAGACGAGACGGCCGGGGTGGCTCG |
| | | CTC |
| SEQ ID | AAV1_Nt.AlwI_BL | AACGGGTGACCTAGAGACGCGCGAGCGAGCGAGCCACCCCGGACGCCT |
| NO: 186 | | GGTTTCCAGGCGTCTGCCGTCTCGAGACGAGACGGCCGGGGTGGCTCG |
| | | CTC |
| SEQ ID | AAV1_Nt.BbvCI_TL | AACGGGTGAGGGCGACTCCCGCGAGCGAGCGAGCCACCCCGGACGCCT |
| NO: 187 | | GGTTTCCAGGCGTCTGCCGTCTCGAGACGAGACGGCCGGGGTGGCTCG |
| | | CTC |
| SEQ ID | AAV1_Nt.BbvCI_BL | AACGGGTGAGGGGGAGTCGCGCGAGCGAGCGAGCCACCCCGGACGCCT |
| NO: 188 | | GGTTTCCAGGCGTCTGCCGTCTCGAGACGAGACGGCCGGGGTGGCTCG |
| | | CTC |
| SEQ ID | AAV1_Nt.BsmAI_TL | AACGGGTGACTCTGAGACGCGCGAGCGAGCGAGCCACCCCGGACGCCT |
| NO: 189 | | GGTTTCCAGGCGTCTGCCGTCTCGAGACGAGACGGCCGGGGTGGCTCG |
| | | CTC |
| SEQ ID | AAV1_Nt.BsmAI_BL | AACGGGTGAGGCAGAGACGCGCGAGCGAGCGAGCCACCCCGGACGCCT |
| NO: 190 | | GGTTTCCAGGCGTCTGCCGTCTCGAGACGAGACGGCCGGGGTGGCTCG |
| | | CTC |
| SEQ ID | AAV1_Nt.BspQI_TL | AACGGGTGAGGGCTTCTCGCGCGAGCGAGCGAGCCACCCCGGACGCCT |
| NO: 191 | | GGTTTCCAGGCGTCTGCCGTCTCGAGACGAGACGGCCGGGGTGGCTCG |
| | | CTC |
| SEQ ID | AAV1_Nt. BspQI_BL | AACGGGTGAGGGCGAGAAGCGCGAGCGAGCGAGCCACCCCGGACGCCT |
| NO: 192 | | GGTTTCCAGGCGTCTGCCGTCTCGAGACGAGACGGCCGGGGTGGCTCG |
| | | CTC |
| SEQ ID | AAV1_Nt.BstNBI_TL | AACGGGTGAGGGAGAGACGCGCTGAGCGAGCGAGCCACCCCGGACGCC |
| NO: 193 | | TGGTTTCCAGGCGTCTGCCGTCTCGAGACGAGACGGCCGGGGTGGCTC |
| | | GCTC |
| SEQ ID | AAV1_Nt.BstNBI_BL | AACGGCTCAGGGAGAGACGCGCGAGCGAGCGAGCCACCCCGGACGCCT |
| NO: 194 | | GGTTTCCAGGCGTCTGCCGTCTCGAGACGAGACGGCCGGGGTGGCTCG |
| | | CTC |
| SEQ ID | wt_AAV2 | AACCGGTGAGGGAGAGACGCGCGAGCGAGCGAGTGACTCCGGCCCGCT |
| NO: 195 | | GGTTTCCAGCGGGCTGCGGGCCCGAAACGGGCCCGCCGGAGTCACTCG |
| | | CTC |
| SEQ ID | AAV2_Nb.BbvCI_BL | AACCGGTGAGGGGGAGTCGCGCGAGCGAGCGAGTGACTCCGGCCCGCT |
| NO: 196 | | GGTTTCCAGCGGGCTGCGGGCCCGAAACGGGCCCGCCGGAGTCACTCG |
| | | CTC |
| SEQ ID | AAV2_Nb.BbvCI_TL | AACCGGTGAGGGCGACTCCCGCGAGCGAGCGAGTGACTCCGGCCCGCT |
| NO: 197 | | GGTTTCCAGCGGGCTGCGGGCCCGAAACGGGCCCGCCGGAGTCACTCG |
| | | CTC |
| SEQ ID | AAV2_Nb.BsmI_BL | AACCGGTGAGGGACTTACGCGCGAGCGAGCGAGTGACTCCGGCCCGCT |
| NO: 198 | | GGTTTCCAGCGGGCTGCGGGCCCGAAACGGGCCCGCCGGAGTCACTCG |
| | | CTC |
| SEQ ID | AAV2_Nb.BsmI_TL | AACCGGTGAGGGAGAGACGCGTAAGCGAGCGAGTGACTCCGGCCCGCT |
| NO: 199 | | GGTTTCCAGCGGGCTGCGGGCCCGAAACGGGCCCGCCGGAGTCACTCG |
| | | CTC |
| SEQ ID | AAV2_Nb.BsrDI_BL | AACCGGTGAGGGCGTTACGCGCGAGCGAGCGAGTGACTCCGGCCCGCT |
| NO: 200 | | GGTTTCCAGCGGGCTGCGGGCCCGAAACGGGCCCGCCGGAGTCACTCG |
| | | CTC |
| SEQ ID | AAV2_Nb.BsrDI_TL | AACCGGTGAGGGAGTAACGCGCGAGCGAGCGAGTGACTCCGGCCCGCT |
| NO: 201 | | GGTTTCCAGCGGGCTGCGGGCCCGAAACGGGCCCGCCGGAGTCACTCG |
| | | CTC |
| SEQ ID | AAV2_Nb.BssSI_BL | AACCGGTGCTCGAGAGACGCGCGAGCGAGCGAGTGACTCCGGCCCGCT |
| NO: 202 | | GGTTTCCAGCGGGCTGCGGGCCCGAAACGGGCCCGCCGGAGTCACTCG |
| | | CTC |
| SEQ ID | AAV2_Nb.BssSI_TL | AACCGGTGAGGGAGCACCGCGCGAGCGAGCGAGTGACTCCGGCCCGCT |
| NO: 203 | | GGTTTCCAGCGGGCTGCGGGCCCGAAACGGGCCCGCCGGAGTCACTCG |
| | | CTC |
| SEQ ID | AAV2_Nb.BtsI_BL | AACCGGTGAGGGCGTCACGCGCGAGCGAGCGAGTGACTCCGGCCCGCT |
| NO: 204 | | GGTTTCCAGCGGGCTGCGGGCCCGAAACGGGCCCGCCGGAGTCACTCG |
| | | CTC |
| SEQ ID | AAV2_Nb.BtsI_TL | AACCGGTGAGGGAGTGACGCGCGAGCGAGCGAGTGACTCCGGCCCGCT |
| NO: 205 | | GGTTTCCAGCGGGCTGCGGGCCCGAAACGGGCCCGCCGGAGTCACTCG |
| | | CTC |
| SEQ ID | AAV2_Nt.AlwI_BL | AACCGGTGAGGGAGCTAGGCGCGAGCGAGCGAGTGACTCCGGCCCGCT |
| NO: 206 | | GGTTTCCAGCGGGCTGCGGGCCCGAAACGGGCCCGCCGGAGTCACTCG |
| | | CTC |
| SEQ ID | AAV2_Nt.AlwI_BL | AACCGGTGACCTAGAGACGCGCGAGCGAGCGAGTGACTCCGGCCCGCT |
| NO: 207 | | GGTTTCCAGCGGGCTGCGGGCCCGAAACGGGCCCGCCGGAGTCACTCG |
| | | CTC |
| SEQ ID | AAV2_Nt.BbvCI_TL | AACCGGTGAGGGCGACTCCCGCGAGCGAGCGAGTGACTCCGGCCCGCT |
| NO: 208 | | GGTTTCCAGCGGGCTGCGGGCCCGAAACGGGCCCGCCGGAGTCACTCG |
| | | CTC |
| SEQ ID | AAV2_Nt.BbvCI_BL | AACCGGTGAGGGGGAGTCGCGCGAGCGAGCGAGTGACTCCGGCCCGCT |
| NO: 209 | | GGTTTCCAGCGGGCTGCGGGCCCGAAACGGGCCCGCCGGAGTCACTCG |
| | | CTC |
| SEQ ID | AAV2_Nt.BsmAI_TL | AACCGGTGACTCTGAGACGCGCGAGCGAGCGAGTGACTCCGGCCCGCT |
| NO: 210 | | GGTTTCCAGCGGGCTGCGGGCCCGAAACGGGCCCGCCGGAGTCACTCG |
| | | CTC |
| SEQ ID | AAV2_Nt.BsmAI_BL | AACCGGTGAGGCAGAGACGCGCGAGCGAGCGAGTGACTCCGGCCCGCT |
| NO: 211 | | GGTTTCCAGCGGGCTGCGGGCCCGAAACGGGCCCGCCGGAGTCACTCG |
| | | CTC |
| SEQ ID | AAV2_Nt.BspQI_TL | AACCGGTGAGGGCTTCTCGCGCGAGCGAGCGAGTGACTCCGGCCCGCT |
| NO: 212 | | GGTTTCCAGCGGGCTGCGGGCCCGAAACGGGCCCGCCGGAGTCACTCG |
| | | CTC |
| SEQ ID | AAV2_Nt. BspQI_BL | AACCGGTGAGGGCGAGAAGCGCGAGCGAGCGAGTGACTCCGGCCCGCT |
| NO: 213 | | GGTTTCCAGCGGGCTGCGGGCCCGAAACGGGCCCGCCGGAGTCACTCG |
| | | CTC |
| SEQ ID | AAV2_Nt.BstNBI_TL | AACCGGTGAGGGAGAGACGCGCTGAGCGAGCGAGTGACTCCGGCCCGC |
| NO: 214 | | TGGTTTCCAGCGGGCTGCGGGCCCGAAACGGGCCCGCCGGAGTCACTC |
| | | GCTC |
| SEQ ID | AAV2_Nt.BstNBI_BL | AACCGCTCAGGGAGAGACGCGCGAGCGAGCGAGTGACTCCGGCCCGCT |
| NO: 215 | | GGTTTCCAGCGGGCTGCGGGCCCGAAACGGGCCCGCCGGAGTCACTCG |
| | | CTC |
| SEQ ID | wt_AAV3 | AACCGGTGAGGGAGATACGCGTGAGCGAGCGAGCCACCCCGGACCGCT |
| NO: 216 | | GGTTTCCAGCGGTCTGCCTGCACGAAACGTGCAGGCCGGGGTGGCTCG |
| | | CTC |
| SEQ ID | AAV3_Nb.BbvCI_BL | AACCGGTGAGGGGGAGTCGCGTGAGCGAGCGAGCCACCCCGGACCGCT |
| NO: 217 | | GGTTTCCAGCGGTCTGCCTGCACGAAACGTGCAGGCCGGGGTGGCTCG |
| | | CTC |
| SEQ ID | AAV3_Nb.BbvCI_TL | AACCGGTGAGGGCGACTCCCGTGAGCGAGCGAGCCACCCCGGACCGCT |
| NO: 218 | | GGTTTCCAGCGGTCTGCCTGCACGAAACGTGCAGGCCGGGGTGGCTCG |
| | | CTC |
| SEQ ID | AAV3_Nb.BsmI_BL | AACCGGTGAGGGACTTACGCGTGAGCGAGCGAGCCACCCCGGACCGCT |
| NO: 219 | | GGTTTCCAGCGGTCTGCCTGCACGAAACGTGCAGGCCGGGGTGGCTCG |
| | | CTC |
| SEQ ID | AAV3_Nb.BsmI_TL | AACCGGTGAGGGAGATACGCGTAAGCGAGCGAGCCACCCCGGACCGCT |
| NO: 220 | | GGTTTCCAGCGGTCTGCCTGCACGAAACGTGCAGGCCGGGGTGGCTCG |
| | | CTC |
| SEQ ID | AAV3_Nb.BsrDI_BL | AACCGGTGAGGGCGTTACGCGTGAGCGAGCGAGCCACCCCGGACCGCT |
| NO: 221 | | GGTTTCCAGCGGTCTGCCTGCACGAAACGTGCAGGCCGGGGTGGCTCG |
| | | CTC |
| SEQ ID | AAV3_Nb.BsrDI_TL | AACCGGTGAGGGAGTAACGCGTGAGCGAGCGAGCCACCCCGGACCGCT |
| NO: 222 | | GGTTTCCAGCGGTCTGCCTGCACGAAACGTGCAGGCCGGGGTGGCTCG |
| | | CTC |
| SEQ ID | AAV3_Nb.BssSI_BL | AACCGGTGCTCGAGATACGCGTGAGCGAGCGAGCCACCCCGGACCGCT |
| NO: 223 | | GGTTTCCAGCGGTCTGCCTGCACGAAACGTGCAGGCCGGGGTGGCTCG |
| | | CTC |
| SEQ ID | AAV3_Nb.BssSI_TL | AACCGGTGAGGGAGCACCGCGTGAGCGAGCGAGCCACCCCGGACCGCT |
| NO: 224 | | GGTTTCCAGCGGTCTGCCTGCACGAAACGTGCAGGCCGGGGTGGCTCG |
| | | CTC |
| SEQ ID | AAV3_Nb.BtsI_BL | AACCGGTGAGGGCGTCACGCGTGAGCGAGCGAGCCACCCCGGACCGCT |
| NO: 225 | | GGTTTCCAGCGGTCTGCCTGCACGAAACGTGCAGGCCGGGGTGGCTCG |
| | | CTC |
| SEQ ID | AAV3_Nb.BtsI_TL | AACCGGTGAGGGAGTGACGCGTGAGCGAGCGAGCCACCCCGGACCGCT |
| NO: 226 | | GGTTTCCAGCGGTCTGCCTGCACGAAACGTGCAGGCCGGGGTGGCTCG |
| | | CTC |
| SEQ ID | AAV3_Nt.AlwI_BL | AACCGGTGAGGGAGCTAGGCGTGAGCGAGCGAGCCACCCCGGACCGCT |
| NO: 227 | | GGTTTCCAGCGGTCTGCCTGCACGAAACGTGCAGGCCGGGGTGGCTCG |
| | | CTC |
| SEQ ID | AAV3_Nt.AlwI_BL | AACCGGTGACCTAGATACGCGTGAGCGAGCGAGCCACCCCGGACCGCT |
| NO: 228 | | GGTTTCCAGCGGTCTGCCTGCACGAAACGTGCAGGCCGGGGTGGCTCG |
| | | CTC |
| SEQ ID | AAV3_Nt.BbvCI_TL | AACCGGTGAGGGCGACTCCCGTGAGCGAGCGAGCCACCCCGGACCGCT |
| NO: 229 | | GGTTTCCAGCGGTCTGCCTGCACGAAACGTGCAGGCCGGGGTGGCTCG |
| | | CTC |
| SEQ ID | AAV3_Nt.BbvCI_BL | AACCGGTGAGGGGGAGTCGCGTGAGCGAGCGAGCCACCCCGGACCGCT |
| NO: 230 | | GGTTTCCAGCGGTCTGCCTGCACGAAACGTGCAGGCCGGGGTGGCTCG |
| | | CTC |
| SEQ ID | AAV3_Nt.BsmAI_TL | AACCGGTGACTCTGATACGCGTGAGCGAGCGAGCCACCCCGGACCGCT |
| NO: 231 | | GGTTTCCAGCGGTCTGCCTGCACGAAACGTGCAGGCCGGGGTGGCTCG |
| | | CTC |
| SEQ ID | AAV3_Nt.BsmAI_BL | AACCGGTGAGGCAGAGACGCGTGAGCGAGCGAGCCACCCCGGACCGCT |
| NO: 232 | | GGTTTCCAGCGGTCTGCCTGCACGAAACGTGCAGGCCGGGGTGGCTCG |
| | | CTC |
| SEQ ID | AAV3_Nt.BspQI_TL | AACCGGTGAGGGCTTCTCGCGTGAGCGAGCGAGCCACCCCGGACCGCT |
| NO: 233 | | GGTTTCCAGCGGTCTGCCTGCACGAAACGTGCAGGCCGGGGTGGCTCG |
| | | CTC |
| SEQ ID | AAV3_Nt.BspQI_BL | AACCGGTGAGGGCGAGAAGCGTGAGCGAGCGAGCCACCCCGGACCGCT |
| NO: 234 | | GGTTTCCAGCGGTCTGCCTGCACGAAACGTGCAGGCCGGGGTGGCTCG |
| | | CTC |
| SEQ ID | AAV3_Nt.BstNBI_TL | AACCGGTGAGGGAGATACGCGCTGAGCGAGCGAGCCACCCCGGACCGC |
| NO: 235 | | TGGTTTCCAGCGGTCTGCCTGCACGAAACGTGCAGGCCGGGGTGGCTC |
| | | GCTC |
| SEQ ID | AAV3_Nt.BstNBI_BL | AACCGCTCAGGGAGATACGCGTGAGCGAGCGAGCCACCCCGGACCGCT |
| NO: 236 | | GGTTTCCAGCGGTCTGCCTGCACGAAACGTGCAGGCCGGGGTGGCTCG |
| | | CTC |
| SEQ ID | wt_AAV4_left | AACCGGTGAGGGAGATACGCGCGAGCGAGTGAGTGAGCCGGGACCTCT |
| NO: 237 | | GGTTTCCAGAGGTCTGACGGCCGGAGACCGGCCGTCCCGGCTCACTCA |
| | | CTCGCTCGCGCGTATCTCCCTCACCGGTT |
| SEQ ID | AAV4_left_Nb.BbvCI_BL | AACCGGTGAGGGGGAGTCGCGCGAGCGAGTGAGTGAGCCGGGACCTCT |
| NO: 238 | | GGTTTCCAGAGGTCTGACGGCCGGAGACCGGCCGTCCCGGCTCACTCA |
| | | CTCGCTCGCGCGACTCCCCCTCACCGGTT |
| SEQ ID | AAV4 left_Nb.BbvCI_TL | AACCGGTGAGGGCGACTCCCGCGAGCGAGTGAGTGAGCCGGGACCTCT |
| NO: 239 | | GGTTTCCAGAGGTCTGACGGCCGGAGACCGGCCGTCCCGGCTCACTCA |
| | | CTCGCTCGCGGGAGTCGCCCTCACCGGTT |
| SEQ ID | AAV4_left_Nb.BsmI_BL | AACCGGTGAGGGACTTACGCGCGAGCGAGTGAGTGAGCCGGGACCTCT |
| NO: 240 | | GGTTTCCAGAGGTCTGACGGCCGGAGACCGGCCGTCCCGGCTCACTCA |
| | | CTCGCTCGCGCGTAAGTCCCTCACCGGTT |
| SEQ ID | AAV4_left_Nb.BsmI_TL | AACCGGTGAGGGAGATACGCGTAAGCGAGTGAGTGAGCCGGGACCTCT |
| NO: 241 | | GGTTTCCAGAGGTCTGACGGCCGGAGACCGGCCGTCCCGGCTCACTCA |
| | | CTCGCTTACGCGTATCTCCCTCACCGGTT |
| SEQ ID | AAV4_left_Nb.BsrDI_BL | AACCGGTGAGGGCGTTACGCGCGAGCGAGTGAGTGAGCCGGGACCTCT |
| NO: 242 | | GGTTTCCAGAGGTCTGACGGCCGGAGACCGGCCGTCCCGGCTCACTCA |
| | | CTCGCTCGCGCGTAACGCCCTCACCGGTT |
| SEQ ID | AAV4_left_Nb.BsrDI_TL | AACCGGTGAGGGAGTAACGCGCGAGCGAGTGAGTGAGCCGGGACCTCT |
| NO: 243 | | GGTTTCCAGAGGTCTGACGGCCGGAGACCGGCCGTCCCGGCTCACTCA |
| | | CTCGCTCGCGCGTTACTCCCTCACCGGTT |
| SEQ ID | AAV4_left_Nb.BssSI_BL | AACCGGTGCTCGAGATACGCGCGAGCGAGTGAGTGAGCCGGGACCTCT |
| NO: 244 | | GGTTTCCAGAGGTCTGACGGCCGGAGACCGGCCGTCCCGGCTCACTCA |
| | | CTCGCTCGCGCGTATCTCGAGCACCGGTT |
| SEQ ID | AAV4 left_Nb.BssSI_TL | AACCGGTGAGGGAGCACCGCGCGAGCGAGTGAGTGAGCCGGGACCTCT |
| NO: 245 | | GGTTTCCAGAGGTCTGACGGCCGGAGACCGGCCGTCCCGGCTCACTCA |
| | | CTCGCTCGCGCGGTGCTCCCTCACCGGTT |
| SEQ ID | AAV4_left_Nb.BtsI_BL | AACCGGTGAGGGCGTCACGCGCGAGCGAGTGAGTGAGCCGGGACCTCT |
| NO: 246 | | GGTTTCCAGAGGTCTGACGGCCGGAGACCGGCCGTCCCGGCTCACTCA |
| | | CTCGCTCGCGCGTGACGCCCTCACCGGTT |
| SEQ ID | AAV4_left_Nb.BtsI_TL | AACCGGTGAGGGAGTGACGCGCGAGCGAGTGAGTGAGCCGGGACCTCT |
| NO: 247 | | GGTTTCCAGAGGTCTGACGGCCGGAGACCGGCCGTCCCGGCTCACTCA |
| | | CTCGCTCGCGCGTCACTCCCTCACCGGTT |
| SEQ ID | AAV4_left_Nt.AlwI_BL | AACCGGTGAGGGAGCTAGGCGCGAGCGAGTGAGTGAGCCGGGACCTCT |
| NO: 248 | | GGTTTCCAGAGGTCTGACGGCCGGAGACCGGCCGTCCCGGCTCACTCA |
| | | CTCGCTCGCGCCTAGCTCCCTCACCGGTT |
| SEQ ID | AAV4_left_Nt.AlwI_BL | AACCGGTGACCTAGATACGCGCGAGCGAGTGAGTGAGCCGGGACCTCT |
| NO: 249 | | GGTTTCCAGAGGTCTGACGGCCGGAGACCGGCCGTCCCGGCTCACTCA |
| | | CTCGCTCGCGCGTATCTAGGTCACCGGTT |
| SEQ ID | AAV4 left_Nt.BbvCI_TL | AACCGGTGAGGGCGACTCCCGCGAGCGAGTGAGTGAGCCGGGACCTCT |
| NO: 250 | | GGTTTCCAGAGGTCTGACGGCCGGAGACCGGCCGTCCCGGCTCACTCA |
| | | CTCGCTCGCGGGAGTCGCCCTCACCGGTT |
| SEQ ID | AAV4_left_Nt.BbvCI_BL | AACCGGTGAGGGGGAGTCGCGCGAGCGAGTGAGTGAGCCGGGACCTCT |
| NO: 251 | | GGTTTCCAGAGGTCTGACGGCCGGAGACCGGCCGTCCCGGCTCACTCA |
| | | CTCGCTCGCGCGACTCCCCCTCACCGGTT |
| SEQ ID | AAV4 left_Nt.BsmAI_TL | AACCGGTGACTCTGATACGCGCGAGCGAGTGAGTGAGCCGGGACCTCT |
| NO: 252 | | GGTTTCCAGAGGTCTGACGGCCGGAGACCGGCCGTCCCGGCTCACTCA |
| | | CTCGCTCGCGCGTATCAGAGTCACCGGTT |
| SEQ ID | AAV4 left_Nt.BsmAI_BL | AACCGGTGAGGCAGAGACGCGCGAGCGAGTGAGTGAGCCGGGACCTCT |
| NO: 253 | | GGTTTCCAGAGGTCTGACGGCCGGAGACCGGCCGTCCCGGCTCACTCA |
| | | CTCGCTCGCGCGTCTCTGCCTCACCGGTT |
| SEQ ID | AAV4_left_Nt.BspQI_TL | AACCGGTGAGGGCTTCTCGCGCGAGCGAGTGAGTGAGCCGGGACCTCT |
| NO: 254 | | GGTTTCCAGAGGTCTGACGGCCGGAGACCGGCCGTCCCGGCTCACTCA |
| | | CTCGCTCGCGCGAGAAGCCCTCACCGGTT |
| SEQ ID | AAV4_left_Nt.BspQI_BL | AACCGGTGAGGGCGAGAAGCGCGAGCGAGTGAGTGAGCCGGGACCTCT |
| NO: 255 | | GGTTTCCAGAGGTCTGACGGCCGGAGACCGGCCGTCCCGGCTCACTCA |
| | | CTCGCTCGCGCTTCTCGCCCTCACCGGTT |
| SEQ ID | AAV4_left_Nt.BstNBI_TL | AACCGGTGAGGGAGATACGCGCTGAGCGAGTGAGTGAGCCGGGACCTC |
| NO: 256 | | TGGTTTCCAGAGGTCTGACGGCCGGAGACCGGCCGTCCCGGCTCACTC |
| | | ACTCGCTCAGCGCGTATCTCCCTCACCGGTT |
| SEQ ID | AAV4 left_Nt.BstNBI_BL | AACCGCTCAGGGAGATACGCGCGAGCGAGTGAGTGAGCCGGGACCTCT |
| NO: 257 | | GGTTTCCAGAGGTCTGACGGCCGGAGACCGGCCGTCCCGGCTCACTCA |
| | | CTCGCTCGCGCGTATCTCCCTGAGCGGTT |
| SEQ ID | wt_AAV4_Right | AACCGGTGTAATCGATACGCGCGAGCGAGTGAGTGAGCCGGGACCTCT |
| NO: 258 | | GGTTTCCAGAGGTCTGACGGCCGGAGACCGGCCGTCCCGGCTCACTCA |
| | | CTCGCTCGCGCGTATCTCCCTCACCGGTT |
| SEQ ID | AAV4_Right_Nb.BbvCI_BL | AACCGGTGTAATGGAGTCGCGCGAGCGAGTGAGTGAGCCGGGACCTCT |
| NO: 259 | | GGTTTCCAGAGGTCTGACGGCCGGAGACCGGCCGTCCCGGCTCACTCA |
| | | CTCGCTCGCGCGACTCCCCCTCACCGGTT |
| SEQ ID | AAV4_Right_Nb.BbvCI_TL | AACCGGTGTAATCGACTCCCGCGAGCGAGTGAGTGAGCCGGGACCTCT |
| NO: 260 | | GGTTTCCAGAGGTCTGACGGCCGGAGACCGGCCGTCCCGGCTCACTCA |
| | | CTCGCTCGCGGGAGTCGCCCTCACCGGTT |
| SEQ ID | AAV4_Right_Nb.BsmI_BL | AACCGGTGTAATCCTTACGCGCGAGCGAGTGAGTGAGCCGGGACCTCT |
| NO: 261 | | GGTTTCCAGAGGTCTGACGGCCGGAGACCGGCCGTCCCGGCTCACTCA |
| | | CTCGCTCGCGCGTAAGTCCCTCACCGGTT |
| SEQ ID | AAV4_Right_Nb.BsmI_TL | AACCGGTGTAATCGATACGCGTAAGCGAGTGAGTGAGCCGGGACCTCT |
| NO: 262 | | GGTTTCCAGAGGTCTGACGGCCGGAGACCGGCCGTCCCGGCTCACTCA |
| | | CTCGCTTACGCGTATCTCCCTCACCGGTT |
| SEQ ID | AAV4_Right_Nb.BsrDI_BL | AACCGGTGTAATCGTTACGCGCGAGCGAGTGAGTGAGCCGGGACCTCT |
| NO: 263 | | GGTTTCCAGAGGTCTGACGGCCGGAGACCGGCCGTCCCGGCTCACTCA |
| | | CTCGCTCGCGCGTAACGCCCTCACCGGTT |
| SEQ ID | AAV4_Right_Nb.BsrDI_TL | AACCGGTGTAATCGTAACGCGCGAGCGAGTGAGTGAGCCGGGACCTCT |
| NO: 264 | | GGTTTCCAGAGGTCTGACGGCCGGAGACCGGCCGTCCCGGCTCACTCA |
| | | CTCGCTCGCGCGTTACTCCCTCACCGGTT |
| SEQ ID | AAV4_Right_Nb.BssSI_BL | AACCGGTGCTCTCGATACGCGCGAGCGAGTGAGTGAGCCGGGACCTCT |
| NO: 265 | | GGTTTCCAGAGGTCTGACGGCCGGAGACCGGCCGTCCCGGCTCACTCA |
| | | CTCGCTCGCGCGTATCTCGAGCACCGGTT |
| SEQ ID | AAV4_Right_Nb.BssSI_TL | AACCGGTGTAAGAGCACCGCGCGAGCGAGTGAGTGAGCCGGGACCTCT |
| NO: 266 | | GGTTTCCAGAGGTCTGACGGCCGGAGACCGGCCGTCCCGGCTCACTCA |
| | | CTCGCTCGCGCGGTGCTCCCTCACCGGTT |
| SEQ ID | AAV4_Right_Nb.BtsI_BL | AACCGGTGTAATCGTCACGCGCGAGCGAGTGAGTGAGCCGGGACCTCT |
| NO: 267 | | GGTTTCCAGAGGTCTGACGGCCGGAGACCGGCCGTCCCGGCTCACTCA |
| | | CTCGCTCGCGCGTGACGCCCTCACCGGTT |
| SEQ ID | AAV4_Right_Nb.BtsI_TL | AACCGGTGTAATCGTGACGCGCGAGCGAGTGAGTGAGCCGGGACCTCT |
| NO: 268 | | GGTTTCCAGAGGTCTGACGGCCGGAGACCGGCCGTCCCGGCTCACTCA |
| | | CTCGCTCGCGCGTCACTCCCTCACCGGTT |
| SEQ ID | AAV4_Right_Nt.AlwI_BL | AACCGGTGTAATCGCTAGGCGCGAGCGAGTGAGTGAGCCGGGACCTCT |
| NO: 269 | | GGTTTCCAGAGGTCTGACGGCCGGAGACCGGCCGTCCCGGCTCACTCA |
| | | CTCGCTCGCGCCTAGCTCCCTCACCGGTT |
| SEQ ID | AAV4_Right_Nt.AlwI_BL | AACCGGTGTCCTAGATACGCGCGAGCGAGTGAGTGAGCCGGGACCTCT |
| NO: 270 | | GGTTTCCAGAGGTCTGACGGCCGGAGACCGGCCGTCCCGGCTCACTCA |
| | | CTCGCTCGCGCGTATCTAGGTCACCGGTT |
| SEQ ID | AAV4_Right_Nt.BbvCI_TL | AACCGGTGTAATCGACTCCCGCGAGCGAGTGAGTGAGCCGGGACCTCT |
| NO: 271 | | GGTTTCCAGAGGTCTGACGGCCGGAGACCGGCCGTCCCGGCTCACTCA |
| | | CTCGCTCGCGGGAGTCGCCCTCACCGGTT |
| SEQ ID | AAV4_Right_Nt.BbvCI_BL | AACCGGTGTAATGGAGTCGCGCGAGCGAGTGAGTGAGCCGGGACCTCT |
| NO: 272 | | GGTTTCCAGAGGTCTGACGGCCGGAGACCGGCCGTCCCGGCTCACTCA |
| | | CTCGCTCGCGCGACTCCCCCTCACCGGTT |
| SEQ ID | AAV4_Right_Nt.BsmAI_TL | AACCGGTGTCTCTGATACGCGCGAGCGAGTGAGTGAGCCGGGACCTCT |
| NO: 273 | | GGTTTCCAGAGGTCTGACGGCCGGAGACCGGCCGTCCCGGCTCACTCA |
| | | CTCGCTCGCGCGTATCAGAGTCACCGGTT |
| SEQ ID | AAV4_Right_Nt.BsmAI_BL | AACCGGTGTAACAGAGACGCGCGAGCGAGTGAGTGAGCCGGGACCTCT |
| NO: 274 | | GGTTTCCAGAGGTCTGACGGCCGGAGACCGGCCGTCCCGGCTCACTCA |
| | | CTCGCTCGCGCGTCTCTGCCTCACCGGTT |
| SEQ ID | AAV4_Right_Nt.BspQI_TL | AACCGGTGTAATCTTCTCGCGCGAGCGAGTGAGTGAGCCGGGACCTCT |
| NO: 275 | | GGTTTCCAGAGGTCTGACGGCCGGAGACCGGCCGTCCCGGCTCACTCA |
| | | CTCGCTCGCGCGAGAAGCCCTCACCGGTT |
| SEQ ID | AAV4_Right_Nt.BspQI_BL | AACCGGTGTAATCGAGAAGCGCGAGCGAGTGAGTGAGCCGGGACCTCT |
| NO: 276 | | GGTTTCCAGAGGTCTGACGGCCGGAGACCGGCCGTCCCGGCTCACTCA |
| | | CTCGCTCGCGCTTCTCGCCCTCACCGGTT |
| SEQ ID | AAV4_Right_Nt.BstNBI_TL | AACCGGTGTAATCGATACGCGCTGAGCGAGTGAGTGAGCCGGGACCTC |
| NO: 277 | | TGGTTTCCAGAGGTCTGACGGCCGGAGACCGGCCGTCCCGGCTCACTC |
| | | ACTCGCTCAGCGCGTATCTCCCTCACCGGTT |
| SEQ ID | AAV4_Right_Nt.BstNBI_BL | AACCGGTCTCAGCGATACGCGCGAGCGAGTGAGTGAGCCGGGACCTCT |
| NO: 278 | | GGTTTCCAGAGGTCTGACGGCCGGAGACCGGCCGTCCCGGCTCACTCA |
| | | CTCGCTCGCGCGTATCTCTGAGACCGGTT |
| SEQ ID | wt_AAV5 | GAGAGGGGGGACAGCGCAAGCGAGCGAGCGACCGAGCAAACCCCCCCA |
| NO: 279 | | CCGTCGAGTTTCTCGACGGTCTGCTGCCGGGAGACCGGCAGCGGGGGG |
| | | GTTTGCTCGGTCGCTCGCTCGCTTGCGCTGTCCCCCCTCTC |
| SEQ ID | AAV5_Nb.BbvCI_BL | GAGAGGGGAGTCGGCGCAAGCGAGCGAGCGACCGAGCAAACCCCCCCA |
| NO: 280 | | CCGTCGAGTTTCTCGACGGTCTGCTGCCGGGAGACCGGCAGCGGGGGG |
| | | GTTTGCTCGGTCGCTCGCTCGCTTGCGCCGACTCCCCTCTC |
| SEQ ID | AAV5_Nb.BbvCI_TL | GAGAGGGGCGACTCCGCAAGCGAGCGAGCGACCGAGCAAACCCCCCCA |
| NO: 281 | | CCGTCGAGTTTCTCGACGGTCTGCTGCCGGGAGACCGGCAGCGGGGGG |
| | | GTTTGCTCGGTCGCTCGCTCGCTTGCGGAGTCGCCCCTCTC |
| SEQ ID | AAV5_Nb.BsmI_BL | GAGAGGGGCTTACGCGCAAGCGAGCGAGCGACCGAGCAAACCCCCCCA |
| NO: 282 | | CCGTCGAGTTTCTCGACGGTCTGCTGCCGGGAGACCGGCAGCGGGGGG |
| | | GTTTGCTCGGTCGCTCGCTCGCTTGCGCGTAAGCCCCTCTC |
| SEQ ID | AAV5_Nb.BsmI_TL | GAGAGGGGGGACAGCGTAAGCGAGCGAGCGACCGAGCAAACCCCCCCA |
| NO: 283 | | CCGTCGAGTTTCTCGACGGTCTGCTGCCGGGAGACCGGCAGCGGGGGG |
| | | GTTTGCTCGGTCGCTCGCTCGCTTACGCTGTCCCCCCTCTC |
| SEQ ID | AAV5_Nb.BsrDI_BL | GAGAGGCGTTACAGCGCAAGCGAGCGAGCGACCGAGCAAACCCCCCCA |
| NO: 284 | | CCGTCGAGTTTCTCGACGGTCTGCTGCCGGGAGACCGGCAGCGGGGGG |
| | | GTTTGCTCGGTCGCTCGCTCGCTTGCGCTGTAACGCCTCTC |
| SEQ ID | AAV5_Nb.BsrDI_TL | GAGAGGGGGTAACGCGCAAGCGAGCGAGCGACCGAGCAAACCCCCCCA |
| NO: 285 | | CCGTCGAGTTTCTCGACGGTCTGCTGCCGGGAGACCGGCAGCGGGGGG |
| | | GTTTGCTCGGTCGCTCGCTCGCTTGCGCGTTACCCCCTCTC |
| SEQ ID | AAV5_Nb.BssSI_BL | GAGTGCTCGGACAGCGCAAGCGAGCGAGCGACCGAGCAAACCCCCCCA |
| NO: 286 | | CCGTCGAGTTTCTCGACGGTCTGCTGCCGGGAGACCGGCAGCGGGGGG |
| | | GTTTGCTCGGTCGCTCGCTCGCTTGCGCTGTCCGAGCACTC |
| SEQ ID | AAV5_Nb.BssSI_TL | GAGAGGGAGCACAGCGCAAGCGAGCGAGCGACCGAGCAAACCCCCCCA |
| NO: 287 | | CCGTCGAGTTTCTCGACGGTCTGCTGCCGGGAGACCGGCAGCGGGGGG |
| | | GTTTGCTCGGTCGCTCGCTCGCTTGCGCTGTGCTCCCTCTC |
| SEQ ID | AAV5_Nb.BtsI_BL | GAGAGGCGTCACAGCGCAAGCGAGCGAGCGACCGAGCAAACCCCCCCA |
| NO: 288 | | CCGTCGAGTTTCTCGACGGTCTGCTGCCGGGAGACCGGCAGCGGGGGG |
| | | GTTTGCTCGGTCGCTCGCTCGCTTGCGCTGTGACGCCTCTC |
| SEQ ID | AAV5_Nb.BtsI_TL | GAGAGGGGTGACGGCGCAAGCGAGCGAGCGACCGAGCAAACCCCCCCA |
| NO: 289 | | CCGTCGAGTTTCTCGACGGTCTGCTGCCGGGAGACCGGCAGCGGGGGG |
| | | GTTTGCTCGGTCGCTCGCTCGCTTGCGCCGTCACCCCTCTC |
| SEQ ID | AAV5_Nt.AlwI_BL | GAGAGGGGGGACAGCGCTAGGGAGCGAGCGACCGAGCAAACCCCCCCA |
| NO: 290 | | CCGTCGAGTTTCTCGACGGTCTGCTGCCGGGAGACCGGCAGCGGGGGG |
| | | GTTTGCTCGGTCGCTCGCTCCCTAGCGCTGTCCCCCCTCTC |
| SEQ ID | AAV5_Nt.AlwI_BL | GAGAGGGGGCCTAGCGCAAGCGAGCGAGCGACCGAGCAAACCCCCCCA |
| NO: 291 | | CCGTCGAGTTTCTCGACGGTCTGCTGCCGGGAGACCGGCAGCGGGGGG |
| | | GTTTGCTCGGTCGCTCGCTCGCTTGCGCTAGGCCCCCTCTC |
| SEQ ID | AAV5_Nt.BbvCI_TL | GAGAGGGGCGACTCCGCAAGCGAGCGAGCGACCGAGCAAACCCCCCCA |
| NO: 292 | | CCGTCGAGTTTCTCGACGGTCTGCTGCCGGGAGACCGGCAGCGGGGGG |
| | | GTTTGCTCGGTCGCTCGCTCGCTTGCGGAGTCGCCCCTCTC |
| SEQ ID | AAV5_Nt.BbvCI_BL | GAGAGGGGAGTCGGCGCAAGCGAGCGAGCGACCGAGCAAACCCCCCCA |
| NO: 293 | | CCGTCGAGTTTCTCGACGGTCTGCTGCCGGGAGACCGGCAGCGGGGGG |
| | | GTTTGCTCGGTCGCTCGCTCGCTTGCGCCGACTCCCCTCTC |
| SEQ ID | AAV5_Nt.BsmAI_TL | GAGAGGGGGCTCTGCGCAAGCGAGCGAGCGACCGAGCAAACCCCCCCA |
| NO: 294 | | CCGTCGAGTTTCTCGACGGTCTGCTGCCGGGAGACCGGCAGCGGGGGG |
| | | GTTTGCTCGGTCGCTCGCTCGCTTGCGCAGAGCCCCCTCTC |
| SEQ ID | AAV5_Nt.BsmAI_BL | GAGAGGGGGCAGAGCGCAAGCGAGCGAGCGACCGAGCAAACCCCCCCA |
| NO: 295 | | CCGTCGAGTTTCTCGACGGTCTGCTGCCGGGAGACCGGCAGCGGGGGG |
| | | GTTTGCTCGGTCGCTCGCTCGCTTGCGCTCTGCCCCCTCTC |
| SEQ ID | AAV5_Nt.BspQI_TL | GAGAGGGCTTCTCGCGCAAGCGAGCGAGCGACCGAGCAAACCCCCCCA |
| NO: 296 | | CCGTCGAGTTTCTCGACGGTCTGCTGCCGGGAGACCGGCAGCGGGGGG |
| | | GTTTGCTCGGTCGCTCGCTCGCTTGCGCGAGAAGCCCTCTC |
| SEQ ID | AAV5_Nt.BspQI_BL | GAGAGGGCGAGAAGCGCAAGCGAGCGAGCGACCGAGCAAACCCCCCCA |
| NO: 297 | | CCGTCGAGTTTCTCGACGGTCTGCTGCCGGGAGACCGGCAGCGGGGGG |
| | | GTTTGCTCGGTCGCTCGCTCGCTTGCGCTTCTCGCCCTCTC |
| SEQ ID | AAV5_Nt.BstNBI_TL | GAGAGGGGGGACAGCGCTGAGCGAGCGAGCGACCGAGCAAACCCCCCC |
| NO: 298 | | ACCGTCGAGTTTCTCGACGGTCTGCTGCCGGGAGACCGGCAGCGGGGG |
| | | GGTTTGCTCGGTCGCTCGCTCGCTCAGCGCTGTCCCCCCTCTC |
| SEQ ID | AAV5_Nt.BstNBI_BL | GAGAGGGGGCTCAGCGCAAGCGAGCGAGCGACCGAGCAAACCCCCCCA |
| NO: 299 | | CCGTCGAGTTTCTCGACGGTCTGCTGCCGGGAGACCGGCAGCGGGGGG |
| | | GTTTGCTCGGTCGCTCGCTCGCTTGCGCTGAGCCCCCTCTC |
| SEQ ID | wt_AAV7 | AACCGGTGAGGGAGATACGCGCGAGCGAGCGAGCCACCCCGGACGCCT |
| NO: 300 | | GGTTTCCAGGCGTCTGCCGTCTCGAGACGAGACGGCCGGGGTGGCTCG |
| | | CTCGCTCGCGCGTATCTCCCTCACCGGTT |
| SEQ ID | AAV7_Nb.BbvCI_BL | AACCGGTGAGGGGGAGTCGCGCGAGCGAGCGAGCCACCCCGGACGCCT |
| NO: 301 | | GGTTTCCAGGCGTCTGCCGTCTCGAGACGAGACGGCCGGGGTGGCTCG |
| | | CTCGCTCGCGCGACTCCCCCTCACCGGTT |
| SEQ ID | AAV7_Nb.BbvCI_TL | AACCGGTGAGGGCGACTCCCGCGAGCGAGCGAGCCACCCCGGACGCCT |
| NO: 302 | | GGTTTCCAGGCGTCTGCCGTCTCGAGACGAGACGGCCGGGGTGGCTCG |
| | | CTCGCTCGCGGGAGTCGCCCTCACCGGTT |
| SEQ ID | AAV7_Nb.BsmI_BL | AACCGGTGAGGGACTTACGCGCGAGCGAGCGAGCCACCCCGGACGCCT |
| NO: 303 | | GGTTTCCAGGCGTCTGCCGTCTCGAGACGAGACGGCCGGGGTGGCTCG |
| | | CTCGCTCGCGCGTAAGTCCCTCACCGGTT |
| SEQ ID | AAV7_Nb.BsmI_TL | AACCGGTGAGGGAGATACGCGTAAGCGAGCGAGCCACCCCGGACGCCT |
| NO: 304 | | GGTTTCCAGGCGTCTGCCGTCTCGAGACGAGACGGCCGGGGTGGCTCG |
| | | CTCGCTTACGCGTATCTCCCTCACCGGTT |
| SEQ ID | AAV7_Nb.BsrDI_BL | AACCGGTGAGGGCGTTACGCGCGAGCGAGCGAGCCACCCCGGACGCCT |
| NO: 305 | | GGTTTCCAGGCGTCTGCCGTCTCGAGACGAGACGGCCGGGGTGGCTCG |
| | | CTCGCTCGCGCGTAACGCCCTCACCGGTT |
| SEQ ID | AAV7_Nb.BsrDI_TL | AACCGGTGAGGGAGTAACGCGCGAGCGAGCGAGCCACCCCGGACGCCT |
| NO: 306 | | GGTTTCCAGGCGTCTGCCGTCTCGAGACGAGACGGCCGGGGTGGCTCG |
| | | CTCGCTCGCGCGTTACTCCCTCACCGGTT |
| SEQ ID | AAV7_Nb.BssSI_BL | AACCGGTGCTCGAGATACGCGCGAGCGAGCGAGCCACCCCGGACGCCT |
| NO: 307 | | GGTTTCCAGGCGTCTGCCGTCTCGAGACGAGACGGCCGGGGTGGCTCG |
| | | CTCGCTCGCGCGTATCTCGAGCACCGGTT |
| SEQ ID | AAV7_Nb.BssSI_TL | AACCGGTGAGGGAGCACCGCGCGAGCGAGCGAGCCACCCCGGACGCCT |
| NO: 308 | | GGTTTCCAGGCGTCTGCCGTCTCGAGACGAGACGGCCGGGGTGGCTCG |
| | | CTCGCTCGCGCGGTGCTCCCTCACCGGTT |
| SEQ ID | AAV7_Nb.BtsI_BL | AACCGGTGAGGGCGTCACGCGCGAGCGAGCGAGCCACCCCGGACGCCT |
| NO: 309 | | GGTTTCCAGGCGTCTGCCGTCTCGAGACGAGACGGCCGGGGTGGCTCG |
| | | CTCGCTCGCGCGTGACGCCCTCACCGGTT |
| SEQ ID | AAV7_Nb.BtsI_TL | AACCGGTGAGGGAGTGACGCGCGAGCGAGCGAGCCACCCCGGACGCCT |
| NO: 310 | | GGTTTCCAGGCGTCTGCCGTCTCGAGACGAGACGGCCGGGGTGGCTCG |
| | | CTCGCTCGCGCGTCACTCCCTCACCGGTT |
| SEQ ID | AAV7_Nt.AlwI_BL | AACCGGTGAGGGAGCTAGGCGCGAGCGAGCGAGCCACCCCGGACGCCT |
| NO: 311 | | GGTTTCCAGGCGTCTGCCGTCTCGAGACGAGACGGCCGGGGTGGCTCG |
| | | CTCGCTCGCGCCTAGCTCCCTCACCGGTT |
| SEQ ID | AAV7_Nt.AlwI_BL | AACCGGTGACCTAGATACGCGCGAGCGAGCGAGCCACCCCGGACGCCT |
| NO: 312 | | GGTTTCCAGGCGTCTGCCGTCTCGAGACGAGACGGCCGGGGTGGCTCG |
| | | CTCGCTCGCGCGTATCTAGGTCACCGGTT |
| SEQ ID | AAV7_Nt.BbvCI_TL | AACCGGTGAGGGCGACTCCCGCGAGCGAGCGAGCCACCCCGGACGCCT |
| NO: 313 | | GGTTTCCAGGCGTCTGCCGTCTCGAGACGAGACGGCCGGGGTGGCTCG |
| | | CTCGCTCGCGGGAGTCGCCCTCACCGGTT |
| SEQ ID | AAV7_Nt.BbvCI_BL | AACCGGTGAGGGGGAGTCGCGCGAGCGAGCGAGCCACCCCGGACGCCT |
| NO: 314 | | GGTTTCCAGGCGTCTGCCGTCTCGAGACGAGACGGCCGGGGTGGCTCG |
| | | CTCGCTCGCGCGACTCCCCCTCACCGGTT |
| SEQ ID | AAV7_Nt.BsmAI_TL | AACCGGTGACTCTGATACGCGCGAGCGAGCGAGCCACCCCGGACGCCT |
| NO: 315 | | GGTTTCCAGGCGTCTGCCGTCTCGAGACGAGACGGCCGGGGTGGCTCG |
| | | CTCGCTCGCGCGTATCAGAGTCACCGGTT |
| SEQ ID | AAV7_Nt.BsmAI_BL | AACCGGTGAGGCAGAGACGCGCGAGCGAGCGAGCCACCCCGGACGCCT |
| NO: 316 | | GGTTTCCAGGCGTCTGCCGTCTCGAGACGAGACGGCCGGGGTGGCTCG |
| | | CTCGCTCGCGCGTCTCTGCCTCACCGGTT |
| SEQ ID | AAV7_Nt.BspQI_TL | AACCGGTGAGGGCTTCTCGCGCGAGCGAGCGAGCCACCCCGGACGCCT |
| NO: 317 | | GGTTTCCAGGCGTCTGCCGTCTCGAGACGAGACGGCCGGGGTGGCTCG |
| | | CTCGCTCGCGCGAGAAGCCCTCACCGGTT |
| SEQ ID | AAV7_Nt.BspQI_BL | AACCGGTGAGGGCGAGAAGCGCGAGCGAGCGAGCCACCCCGGACGCCT |
| NO: 318 | | GGTTTCCAGGCGTCTGCCGTCTCGAGACGAGACGGCCGGGGTGGCTCG |
| | | CTCGCTCGCGCTTCTCGCCCTCACCGGTT |
| SEQ ID | AAV7_Nt.BstNBI_TL | AACCGGTGAGGGAGATACGCGCTGAGCGAGCGAGCCACCCCGGACGCC |
| NO: 319 | | TGGTTTCCAGGCGTCTGCCGTCTCGAGACGAGACGGCCGGGGTGGCTC |
| | | GCTCGCTCAGCGCGTATCTCCCTCACCGGTT |
| SEQ ID | AAV7_Nt.BstNBI_BL | AACCGCTCAGGGAGATACGCGCGAGCGAGCGAGCCACCCCGGACGCCT |
| NO: 320 | | GGTTTCCAGGCGTCTGCCGTCTCGAGACGAGACGGCCGGGGTGGCTCG |
| | | CTCGCTCGCGCGTATCTCCCTGAGCGGTT |

The first, second, third, and fourth restriction sites for nicking endonuclease can be arranged in various configurations. In certain embodiments, the first and the second restriction sites for nicking endonuclease are at least 10, at least 15, at least 20, at least 25, at least 30, at least 35, at least 40, at least 45, at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, at least 99, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, or at least 200 nucleotides apart. In certain embodiments, the first and the second restriction sites for nicking endonuclease are about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 105, about 110, about 115, about 120, about 125, about 130, about 135, about 140, about 145, about 150, about 155, about 160, about 165, about 170, about 175, about 180, about 185, about 190, about 195, or about 200 nucleotides apart.

Similarly, in certain embodiments, the third and the fourth restriction sites for nicking endonuclease are at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, or at least 200 nucleotides apart. In certain embodiments, the third and the fourth restriction sites for nicking endonuclease are about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 105, about 110, about 115, about 120, about 125, about 130, about 135, about 140, about 145, about 150, about 155, about 160, about 165, about 170, about 175, about 180, about 185, about 190, about 195, or about 200 nucleotides apart.

Overhangs described in Sections 5.3 (including 5.3.3), and 5.1.1 (including 5.1.1(a)) can result from the nicking at the first and second restriction sites by nicking endonucleases and denaturing as described in Sections 5.3 (including 5.3.3). Thus, in certain embodiments, the overhang resulted from the nicking at the first and second restriction sites can be the same length as the first and second restriction sites are apart (in number of nucleotides) as described in the preceding paragraphs of this Section (Section 5.1.1(b)). As the nicking endonucleases can cut the DNA within or outside the restriction sites for the nicking endonucleases, in certain embodiments, the overhang resulted from the nicking at the first and second restriction sites can be longer or shorter than the first and second restriction sites are apart by at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides. In certain embodiments, the overhang resulted from the nicking at the first and second restriction sites can be longer or shorter than the first and second restriction sites are apart by about 10, about 11, about 12, about 13, about 14, about 15, about 16, about 17, about 18, about 19, about 20, about 21, about 22, about 23, about 24, about 25, about 26, about 27, about 28, about 29, or about 30 nucleotides.

Similarly, overhangs described in Sections 5.3 (including Section 5.3.3), and 5.1.1 (including Section 5.1.1(a)) can be the result of the nicking at the third and fourth restriction sites by nicking endonucleases and denaturing as described in Sections 5.3 (including Section 5.3.3). Thus, in certain embodiments, the overhang resulted from the nicking at the third and fourth restriction sites can be the same length as the third and fourth restriction sites are apart (in number of nucleotides) as described in the preceding paragraphs of this Section (Section 5.1.1(b)). As the nicking endonucleases can cut the DNA within or outside the restriction sites for the nicking endonucleases, in certain embodiments, the overhang resulted from the nicking at the third and fourth restriction sites can be longer or shorter than the third and fourth restriction sites are apart by at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides. In certain embodiments, the overhang resulted from the nicking at the third and fourth restriction sites can be longer or shorter than the third and fourth restriction sites are apart by about 10, about 11, about 12, about 13, about 14, about 15, about 16, about 17, about 18, about 19, about 20, about 21, about 22, about 23, about 24, about 25, about 26, about 27, about 28, about 29, or about 30 nucleotides.

In certain embodiments, the hairpin-ended DNA molecules provided herein comprise a sequence of interest (Section 5.1.1(c)). In certain embodiments, the sequence of interest is located in the segment where the first and second restriction sites for nicking endonuclease(s) at one end and the third and fourth restriction sites for nicking endonuclease(s) at the other end. In certain embodiments, the sequence of interest is located within the dsDNA segment of the DNA molecules produced by performing the steps (e.g., denaturing step) described in Section 5.3 (including Section 5.3.3) to produce two ssDNA overhangs. In certain embodiments, the first, second, third, and fourth restriction sites for the nicking endonucleases are arranged such that the length of the dsDNA segment described in this paragraph is at least 0.2 kb, at least 0.3 kb, at least 0.4 kb, at least 0.5 kb, at least 0.6, at least kb, at least 0.7 kb, at least 0.8 kb, at least 0.9 kb, at least 1 kb, at least 1.5 kb, at least 2 kb, at least 2.5 kb, at least 3 kb, at least 3.5 kb, at least 4 kb, at least 4.5 kb, at least 5 kb, at least 5.5 kb, at least 6 kb, at least 6.5 kb, at least 7 kb, at least 7.5 kb, at least 8 kb, at least 8.5 kb, at least 9 kb, at least 9.5 kb, or at least 10 kb. In certain embodiments, the first, second, third, and fourth restriction sites for the nicking endonucleases are arranged such that the length of the dsDNA segment described in this paragraph is about 0.2 kb, about 0.3 kb, about 0.4 kb, about 0.5 kb, about 0.6, about 0.7 kb, about 0.8 kb, about 0.9 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, or about 10 kb.

As described in Section 5.3.2, incubation with nicking endonucleases will result in a first nick corresponding to the first restriction site for the nicking endonuclease, a second nick corresponding to the second restriction site for the nicking endonuclease, a third nick corresponding to the third restriction site for the nicking endonuclease, and/or a fourth nick corresponding to the fourth restriction site for the nicking endonuclease. The first, second, third, and/or fourth nicks can be at various positions relative to the inverted repeat. In certain embodiments, the first nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 5′ nucleotide of the ITR closing base pair of the first inverted repeat. In certain embodiments, the first nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 3′ nucleotide of the ITR closing base pair of the first inverted repeat. In certain embodiments, the second nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 5′ nucleotide of the ITR closing base pair of the first inverted repeat. In certain embodiments, the second nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 3′ nucleotide of the ITR closing base pair of the first inverted repeat. In certain embodiments, the third nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 5′ nucleotide of the ITR closing base pair of the second inverted repeat. In certain embodiments, the third nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 3′ nucleotide of the ITR closing base pair of the second inverted repeat. In certain embodiments, the fourth nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 5′ nucleotide of the ITR closing base pair of the second inverted repeat. In certain embodiments, the fourth nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 3′ nucleotide of the ITR closing base pair of the second inverted repeat. In certain embodiments, any combinations of the first, second, third, and fourth nicks are inside the inverted repeat. In certain embodiments, any combinations of the first, second, third, and fourth nicks are outside the inverted repeat. In certain embodiments, the first, second, third, and fourth nicks can have any relative positions amongst themselves, between any of them and the inverted repeat, and/or between any of them and the sequence of interest, in any combination or permutation. In some certain embodiments, the first, second, third, and fourth restriction sites for nicking endonucleases can have any relative positions amongst themselves, between any of them and the inverted repeat, and/or between any of them and the sequence of interest, in any combination or permutation.

### (c) Sequence of Interest

In certain embodiments, the DNA templates disclosed herein, amplification products and hairpin-ended DNA molecules produced therefrom comprise a sequence of interest. Any sequence of interest can be included in the DNA molecules disclosed herein. In certain embodiments, the sequence of interest can be a therapeutic or a diagnostic sequence. In certain embodiments, the sequence of interest is flanked by the hairpin ends on either side of the sequence of interest. Examples of the sequences of interest are provided in the sections below.

In certain embodiments, the sequence of interest encodes a peptide or protein that is itself diagnostic or therapeutic. In certain embodiments, the sequence of interest encodes a diagnostic or therapeutic RNA molecule that is transcribed from the DNA sequence of interest. In certain embodiments, the sequence of interest encodes a Rep and/or a Cap of an AAV vector. In certain embodiments, the sequence of interest encodes a component of a helper plasmid. In certain embodiments, the sequence of interest encodes a component of a CRISPR/Cas system. In certain embodiments, the sequence of interest comprises a gene promoter (e.g., a T7 promoter), an AAV ITR, or a synthetic DNA template to be integrated into a genome by a gene engineering technique (e.g., CRISPR/Cas system, transposase). In certain embodiments, the non-sequence of interest comprises a gene promoter (e.g., a T7 promoter), an AAV ITR, or a synthetic DNA template to be integrated into a genome by a gene engineering technique (e.g., CRISPR/Cas system, transposase). In certain embodiments, the sequence of interest encodes a promoter operably linked to a transgene flanked by 5′ and 3′ LTRs such that the hairpin-ended DNA is suitable as a lentivirus transfer vector.

In certain embodiments, the sequence of interest encodes at least one component of a viral genome. In certain embodiments, the sequence of interest encodes at least one component of an AAV genome, a lentiviral genome, or an adenoviral genome.

In certain embodiments, the sequence of interest encodes a synthetic DNA template to be integrated into a genome by a gene engineering technique. In certain embodiments, the synthetic DNA template comprises an expression cassette described in Section 5.1.1(c). In some embodiments, the expression cassette encodes a ORF operably linked to a promoter. In some embodiments, the expression cassette encodes a ORF operably linked to an intron (e.g., to exploit the expression of the targeted locus). In some embodiments, the expression cassette encodes a ORF operably linked to a fragment of an intron that includes the splicing acceptor. In some embodiments, the expression cassette encodes a ORF operably linked to a IRES and/or self-cleaving peptide, such as a 2A peptide (e.g., to exploit the expression of the targeted locus).

In certain embodiments, the sequence of interest (e.g., a sequence as exemplified in the Expression Cassette, CRISPR, AAV Vector, Lentivirus Vector, and RNA sections below) is at least 0.2 kb, at least 0.3 kb, at least 0.4 kb, at least 0.5 kb, at least 0.6, at least kb, at least 0.7 kb, at least 0.8 kb, at least 0.9 kb, at least 1 kb, at least 1.5 kb, at least 2 kb, at least 2.5 kb, at least 3 kb, at least 3.5 kb, at least 4 kb, at least 4.5 kb, at least 5 kb, at least 5.5 kb, at least 6 kb, at least 6.5 kb, at least 7 kb, at least 7.5 kb, at least 8 kb, at least 8.5 kb, at least 9 kb, at least 9.5 kb, at least 10 kb, at least 15 kb, at least 20 kb, at least 25 kb, at least 30 kb, at least 35 kb, at least 40 kb, at least 45 kb, at least 50 kb, at least 55 kb, at least 60 kb, at least 65 kb, at least 70 kb, at least 75 kb, or at least 80 kb. In certain embodiments, the size of the sequence of interest is about 0.2 kb, about 0.3 kb, about 0.4 kb, about 0.5 kb, about 0.6 kb, about 0.7 kb, about 0.8 kb, about 0.9 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, about 10 kb, about 15 kb, about 20 kb, about 25 kb, about 30 kb, about 35 kb, about 40 kb, about 45 kb, about 50 kb, about 55 kb, about 60 kb, about 65 kb, about 70 kb, about 75 kb, or about 80 kb.

The sequence of interest (e.g., a sequence as exemplified in the Expression Cassette, CRISPR, AAV Vector, Lentivirus Vector, and RNA sections below) can have various positions relative to the inverted repeats that flank the sequence of interest. In certain embodiments, the sequence of interest is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 nucleotides apart from one or from both inverted repeats. In certain embodiments, the sequence of interest is at least 0.2 kb, at least 0.3 kb, at least 0.4 kb, at least 0.5 kb, at least 0.6, at least 0.7 kb, at least 0.8 kb, at least 0.9 kb, at least 1 kb, at least 1.5 kb, at least 2 kb, at least 3 kb, at least 4 kb, or at least 5 kb apart from one or both inverted repeats.

In certain embodiments, the sequence of interest (e.g., a sequence as exemplified in the Expression Cassette, CRISPR, AAV Vector, Lentivirus Vector, and RNA sections below) is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 nucleotides apart from one or both inverted repeats. In further embodiments, the sequence of interest is about 0.2 kb, about 0.3 kb, about 0.4 kb, about 0.5 kb, about 0.6 kb, about 0.7 kb, about 0.8 kb, about 0.9 kb, about 1 kb, about 1.5 kb, about 2 kb, about 3 kb, about 4 kb, or about 5 kb apart from the inverted repeat. The distances specified in this paragraph can be independently chosen for the 5′ located and/or the 3′ located inverted repeat. In certain embodiments, both distances are about (i.e., within +/−10%) the same.

In certain embodiments, the sequence of interest is at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, or at most 100 nucleotides apart from one or from both inverted repeats. In certain embodiments, the sequence of interest is at most 0.2 kb, at most 0.3 kb, at most 0.4 kb, at most 0.5 kb, at most 0.6, at most 0.7 kb, at most 0.8 kb, at most 0.9 kb, at most 1 kb, at most 1.5 kb, at most 2 kb, at most 3 kb, at most 4 kb, or at most 5 kb apart from one or both inverted repeats. The distances specified in this paragraph can be independently chosen for the 5′ located and/or the 3′ located inverted repeat. In certain embodiments, both distances are about (i.e., within +/−10%) the same.

In certain embodiments, the inverted repeat is the first inverted repeat as described in Section 5.1.1(a). In certain embodiments, the inverted repeat is the second inverted repeat as described in Section 5.1.1(a). In certain embodiments, the inverted repeat is both the first and the second inverted repeat as described in Section 5.1.1(a).

In certain embodiments, one or more nucleotide sequence(s) located between the sequence of interest and the 5′ or 3′ inverted repeats is a “spacer” comprising one or more non-coding sequence(s). In one embodiment, the spacer sequence has a desired secondary structure. In one embodiment, the spacer sequence has a desired CG content. In one embodiment, the spacer sequence lacks any CpG motifs. In one embodiment, the spacer sequence comprises the nucleotides separating the 5′ or 3′ inverted repeats and the sequence of interest. In one embodiment, the hairpin-ended DNA comprises two spacer sequences separated by a multiple cloning site. In one embodiment, the sequence of a first spacer between the first inverted repeat (e.g. the 5′ inverted repeat) and the sequence of interest is different than the sequence of a second spacer between the sequence of interest and the second inverted repeat (e.g. the 3′ inverted repeat). In one embodiment, the hairpin-ended DNA comprises at least one spacer of natural origin. In one embodiment, the hairpin-ended DNA comprises at least one fully synthetic spacer. In one embodiment, the hairpin-ended DNA comprises at least one spacer that is a chimera between sequences from natural and synthetic origin.

In one embodiment, the hairpin-ended DNA comprises one or more spacers suitable for use as homology arms for CRISPR-mediated HDR of a genome. In one embodiment, the sequence of one or more of the spacers is designed as homology arms for CRISPR-mediated HDR. In one embodiment, each spacer is a homology arm targeting two different sites in the genome. In one embodiment, the two targeting sites are adjacent to each other in the genome and the gRNA target is removed after homologous direct repair. In some embodiments, the two targeting sites are are separated by about 1-20, 20-100, 100-500, 500-1000, 1000-2000, 2000-5000, or more than 5000 nucleotides in the genome. In one embodiment, the homology arm comprises 300-1000 nucleotides. In a preferred embodiment, the homology arm comprises between 300-800 nucleotides. In one embodiment, the sequence of the spacers can be targeted by a gRNA to make CRISPR-mediated double stranded DNA break. In one embodiment, the sequence of the spacers is selected as a non-coding sequence having a desired secondary structure and/or homology arms.

The various embodiments described herein with nicking endonucleases and/or restriction sites, nicking endonucleases are additionally provided with nicking endonucleases replaced by programmable nicking enzyme and restriction sites replaced by targeting sites for programmable nicking enzyme. Exemplary programmable nicking enzymes and their targeting sites have been provided in Section 5.3.2.

In some embodiments, the hairpin-ended DNA molecules do not have the size limitations of encapsidated AAV vectors, thus enabling delivery of a large-size sequences of interest (e.g., a sequence as exemplified in the Expression Cassette, CRISPR, RNA, and Additional Sequences sections below). In certain embodiments, the hairpin-ended DNA molecules comprise a sequence of interest equal to or larger than the size of any natural AAV genome.

### (i) Expression Cassettes

In certain embodiments, a hairpin-ended provided herein can comprise an expression cassette. An “expression cassette” is a nucleic acid molecule or a part of nucleic acid molecule containing sequences or other information that directs the cellular machinery to make RNA, which, in certain embodiments, can be translated into protein. In certain embodiments, the expression cassette comprises a transcription unit. In certain embodiments, the expression cassette comprises two or more transcription units (e.g., 2, 3, 4, 5, 6, 7, 8, 9, 10, 11, 12, 13, 14, 15, 16, 17, 18, 19, or 20 transcription units).

In certain embodiments, the transcription unit comprises a promoter sequence. In certain embodiments, the transcription unit comprises an open reading frame (ORF). In certain embodiments, the transcription unit comprises a promoter operatively linked to an ORF. Embodiments for ORFs for use with the methods and compositions provided herein are further described at the end of the instant Section 5.1.1(c)(i). The transcription unit can further comprise features to direct the cellular machinery to make RNA and protein. In certain embodiments, the transcription unit comprises a posttranscriptional regulatory element. In certain embodiments, the transcription unit further comprises a polyadenylation and/or termination signal. In certain embodiments, the poly-adenylation is directly encoded in the transcription unit such that the transcript is directly synthesized with a polyadenylation sequence. In certain embodiments, the transcription unit comprises regulatory elements known and used in the art to regulate (e.g., promote, inhibit and/or turn on/off the expression of the ORF). Such regulatory elements include, for example, 5′-untranslated region (UTR), 3′-UTR, or both the 5′UTR and the 3′UTR. In some further embodiments, the transcription unit comprises any one or more features provided in the instant Section 5.1.1(c)(i) in any combination or permutation. The ORF (sense strand) can comprise a protein coding sequence. Alternatively, the transcription unit can comprise the complementary sequence of the protein coding ORF (anti-sense strand) and the regulatory components and/or other signals for the cellular machinery to produce a sense strand DNA/RNA and the corresponding protein. In certain embodiments, the transcription unit comprises a protein-encoding sequence without intron sequence. In certain embodiments, the transcription unit comprises a protein-encoding sequence with intron. In certain embodiments, the intron is removed upon transcription and splicing. In certain embodiments, the ORF comprises at least two protein-encoding sequences operably linked by a self-cleaving peptide (such as a 2A peptide). In certain embodiments, the intron is not removed upon transcription, wherein the transcription unit encodes a non-coding RNA (ncRNA). In certain embodiments, the transcription unit comprises any combination of components disclosed in this paragraph (e.g., ORFs, promoters, regulatory elements, poly-adenylation, terminal signal, etc.). The transcription unit can also comprise various numbers of ORFs. The transcription unit can have at least one promoter operably linked to a multicistronic or bicistronic sequence for the co-expression of multiple ORFs and/or ncRNA from a single transcript. In certain embodiments, the multicistronic or bicistronic sequence comprises an internal ribosome entry site (IRES) between each ORF.

The transcription unit can also comprise one or more regulatory elements, one or more transcriptional regulatory elements, one or more posttranscriptional regulatory elements, or any combinations thereof. Such regulatory elements are any sequences that allow, contribute or modulate the functional regulation of the nucleic acid molecule, including replication, duplication, transcription, splicing, translation, stability and/or transport of the nucleic acid or one of its derivatives (e.g., mRNA) into the host cell or organism. Such regulatory elements include, but are not limited to, a promoter, an enhancer, a polyadenylation signal, a translation stop codon, a ribosome binding element, a transcription terminator, selection markers, origin of replication, etc.

In certain embodiments, the transcription unit comprises an enhancer. Any enhancer sequence known to those skilled in the art in view of the present disclosure can be used. In certain embodiments, an enhancer sequence can be human actin, human myosin, human hemoglobin, human muscle creatine, or a viral enhancer, such as one from CMV, HA, RSV, or EBV. In certain specific embodiments, the enhancer sequence can be Woodchuck HBV Posttranscriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit β-globin intron, a P5 promoter of an AAV, or any combination thereof. In certain embodiments, the enhancer sequence is from mouse. In certain embodiments, the enhancer sequence is from human.

As described above, the transcription unit can comprise a promoter to control expression of a protein of interest. Promoters include any nucleotide sequence that initiates the transcription of an operably linked nucleotide sequence. Promoters can be a constitutive, inducible, or repressible. A promoter can be derived from sources including viral, bacterial, fungal, plants, insects, and animals. A promoter can be a homologous promoter (e.g., derived from the same genetic source as the operably linked nucleotide sequence) or a heterologous promoter (e.g., derived from a different genetic source from the operably linked nucleotide sequence). In certain embodiments, a promoter can be a promoter from simian virus (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), an Epstein Barr virus (EBV) promoter, or a Rous sarcoma virus (RSV) promoter. In certain embodiments, a promoter can be a promoter from a human gene such as human actin, human myosin, human hemoglobin, human muscle creatine, or human metallothionein. In further embodiments, a promoter can also be a tissue specific promoter, such as a muscle or skin specific promoter, natural or synthetic.

As described above, the transcription unit can comprise a polyadenylation, termination signal, or both a polyadenylation and termination signal. Any polyadenylation signal known to those skilled in the art in view of the present disclosure can be used. In certain embodiments, the polyadenylation signal can be a SV40 polyadenylation signal, AAV2 polyadenylation signal (bp 4411-4466, NC_001401), a polyadenylation signal from the Herpes Simplex Virus Thymidine Kinase Gene, LTR polyadenylation signal, bovine growth hormone (bGH) polyadenylation signal, human growth hormone (hGH) polyadenylation signal, or human β-globin polyadenylation signal. In certain embodiments, the polyadenylation sequence is directly encoded by the expression cassette such that the primary transcript comprises a polyadenylation sequence without the need for further processing.

In some embodiments, the polyadenylation sequence is a homopolymeric sequence comprising an uninterrupted polyA sequence. In certain embodiments, the homopolymeric sequence is between 30-200 nucleotides in length. In certain embodiments, the homopolymeric sequence is at least 200 nucleotides in length. In some embodiments, the polyadenylation sequence is a homopolymeric sequence comprising two or more polyA sequences interrupted by at least one non-polyA sequence. In certain embodiments, each segment of polyA sequences is between 30-100 nucleotides in length. In certain embodiments, the segment of non-polyA sequences is between 1-15 nucleotides in length.

The expression cassette can have various sizes to accommodate one or more ORFs of various lengths. In certain embodiments, the size of expression cassette is at least 0.2 kb, at least 0.3 kb, at least 0.4 kb, at least 0.5 kb, at least 0.6, at least kb, at least 0.7 kb, at least 0.8 kb, at least 0.9 kb, at least 1 kb, at least 1.5 kb, at least 2 kb, at least 2.5 kb, at least 3 kb, at least 3.5 kb, at least 4 kb, at least 4.5 kb, at least 5 kb, at least 5.5 kb, at least 6 kb, at least 6.5 kb, at least 7 kb, at least 7.5 kb, at least 8 kb, at least 8.5 kb, at least 9 kb, at least 9.5 kb, at least 10 kb, at least 15 kb, at least 20 kb, at least 25 kb, at least 30 kb, at least 35 kb, at least 40 kb, at least 45 kb, at least 50 kb, at least 55 kb, at least 60 kb, at least 65 kb, at least 70 kb, at least 75 kb, or at least 80 kb. In certain embodiments, the expression cassette is at least 4.5 kb. In certain embodiments, the expression cassette is at least 4.6 kb. In yet certain embodiments, the expression cassette is at least 4.7 kb. In certain embodiments, the expression cassette is at least 4.8 kb. In certain embodiments, the expression cassette is at least 4.9 kb. In certain embodiments, the expression cassette is at least 5 kb. In certain embodiments, the size of the expression cassette is about 0.2 kb, about 0.3 kb, about 0.4 kb, about 0.5 kb, about 0.6 kb, about 0.7 kb, about 0.8 kb, about 0.9 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, about 10 kb, about 15 kb, about 20 kb, about 25 kb, about 30 kb, about 35 kb, about 40 kb, about 45 kb, about 50 kb, about 55 kb, about 60 kb, about 65 kb, about 70 kb, about 75 kb, or about 80 kb. In certain embodiments, the expression cassette is about 4.5 kb. In certain embodiments, the expression cassette is about 4.6 kb. In yet certain embodiments, the expression cassette is about 4.7 kb. In certain embodiments, the expression cassette is about 4.8 kb. In certain embodiments, the expression cassette is about 4.9 kb. In certain embodiments, the expression cassette is about 5 kb. The expression cassette can also comprise various numbers of genes of interest (“transgenes”). In certain embodiments, the expression cassette comprises 1, 2, 3, 4, 5, 6, 7, 8, 9, 10, 11, 12, 13, 14, 15, 16, 17, 18, 19, or 20 transgenes. In a specific embodiment, the expression cassette comprises one transgene. In certain embodiments, the transgenes are recombinant genes. In some further embodiments, the transgenes comprise cDNA sequences (e.g., no introns in the transgenes).

In certain embodiments, the expression cassette can comprise a transgene in the range of from about 500 to about 50,000 nucleotides in length. In certain embodiments, the expression cassette can comprise a transgene in the range of from about 500 to about 75,000 nucleotides in length. In certain embodiments, the expression cassette can comprise a transgene that is in the range of from about 500 to about 10,000 nucleotides in length. In certain embodiments, the expression cassette can comprise a transgene that is in the range of from about 1000 to about 10,000 nucleotides in length. In certain embodiments, the expression cassette can comprise a transgene that is in the range of from about 500 to about 5,000 nucleotides in length. In some embodiments, the hairpin-ended DNA molecules do not have the size limitations of encapsidated AAV vectors, thus enabling delivery of a large-size expression cassette to provide efficient transgene expression. In certain embodiments, the hairpin-ended DNA molecules comprise an expression cassette equal to or larger than the size of any natural AAV genome.

The expression cassette can have various positions relative to the inverted repeats that flank the expression cassette. In certain embodiments, the expression cassette is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 nucleotides apart from one or from both inverted repeats. In certain embodiments, the expression cassette is at least 0.2 kb, at least 0.3 kb, at least 0.4 kb, at least 0.5 kb, at least 0.6, at least 0.7 kb, at least 0.8 kb, at least 0.9 kb, at least 1 kb, at least 1.5 kb, or at least 2 kb apart from one or both inverted repeats.

In certain embodiments, the expression cassette is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 nucleotides apart from one or both inverted repeats. In further embodiments, the expression cassette is about 0.2 kb, about 0.3 kb, about 0.4 kb, about 0.5 kb, about 0.6 kb, about 0.7 kb, about 0.8 kb, about 0.9 kb, about 1 kb, about 1.5 kb, or about 2 kb apart from the inverted repeat. The distances specified in this paragraph can be independently chosen for the 5′ located and/or the 3′ located inverted repeat (referring to the open reading frame in the expression cassette in sense direction). In certain embodiments, both distances are about (i.e., within +/−10%) the same.

The expression cassette can have various positions relative to the inverted repeats that flank the expression cassette. In certain embodiments, the expression cassette is at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, or at most 100 nucleotides apart from one or from both inverted repeats. In certain embodiments, the expression cassette is at most 0.2 kb, at most 0.3 kb, at most 0.4 kb, at most 0.5 kb, at most 0.6, at most 0.7 kb, at most 0.8 kb, at most 0.9 kb, at most 1 kb, at most 1.5 kb, or at most 2 kb apart from one or both inverted repeats. The distances specified in this paragraph can be independently chosen for the 5′ located and/or the 3′ located inverted repeat (referring to the open reading frame in the expression cassette in sense direction). In certain embodiments, both distances are about (i.e., within +/−10%) the same.

In certain embodiments, the inverted repeat is the first inverted repeat as described in Section 5.1.1(a). In certain embodiments, the inverted repeat is the second inverted repeat as described in Section 5.1.1(a). In certain embodiments, the inverted repeat is both the first and the second inverted repeat as described in Section 5.1.1(a).

The expression cassette can comprise one or more ORFs. In certain embodiments, the ORF is an ORF of a human gene wherein genetic mutations in the human gene are known to cause a disease. In certain embodiments, the ORF is an ORF of a human gene wherein genetic mutations in the human gene are known to cause a hereditary disease. In certain embodiments, the ORF encodes a therapeutic protein. In certain embodiments, the ORF encodes an enzyme. In certain embodiments, the ORF encodes a metabolic enzyme. In certain embodiments, the ORF encodes an enzyme, wherein the enzyme replaces or supplements the function of a defective enzyme in human. In certain embodiments, the ORF encodes an antibody. In certain embodiments, the ORF encodes a therapeutic antibody. In certain embodiments, the ORF encodes a cytokine. In certain embodiments, the ORF encodes a RNA. In certain embodiments, the ORF encodes a regulatory RNA. In certain embodiments, the ORF encodes an anti-sense RNA. In certain embodiments, the ORF encodes a siRNA. In certain embodiments, the ORF encodes a shRNA. In certain embodiments, the ORF encodes a miRNA. In certain embodiments, the ORF encodes a piRNA (PIWI-interacting RNA). In certain embodiments, the ORF is an ORF of a non-human gene. In certain embodiments, the expression cassette comprises any one or more features described in the instant Section 5.1.1(c)(i) in various permutations and combinations.

In some embodiments, the hairpin-ended DNA molecules do not have the size limitations of encapsidated AAV vectors, thus enabling delivery of a large-size expression cassette to provide efficient transgene expression. In certain embodiments, the hairpin-ended DNA molecules comprise an expression cassette equal to or larger than the size of any natural AAV genome.

### (ii) CRISPR/Cas System

In certain embodiments, the sequence of interest that is comprised within a hairpin-ended DNA molecule provided herein encodes an element for use with CRISPR/Cas system. In certain embodiments, the DNA of interest comprises the ORF for the CRISPR-associated endonuclease Cas9 protein. Expression of the Cas9 protein from a DNA of interest can be under the control of regulatory elements as described above. The Cas9 open reading frame can be part of an expression cassette as described above. In certain embodiments, a guide RNA can be transcribed from the DNA of interest that is comprised by the hairpin-ended DNA molecule provided herein. Transcription of such a guide RNA can be under the control of regulatory elements as described above. In certain embodiments, a Cas9 open reading frame and a guide RNA are comprised by the same hairpin-ended DNA molecule. In certain embodiments, a Cas9 open reading frame and a guide RNA are comprised by different hairpin-ended DNA molecules.

In certain embodiments, the sequence of interest comprises a DNA sequence to be integrated into a target site by the CRISPR/Cas system or other gene engineering system known in the art. In certain embodiments, the DNA sequence is a synthetic DNA template.

Size and location of the sequence of the element for use with CRISPR can be as described above.

(iii) RNA

In certain embodiments, an RNA molecule (e.g., therapeutic or diagnostic RNA molecules) can be transcribed from a sequence of interest that is comprised by a hairpin-ended DNA molecule provided herein. In certain embodiments, the RNA molecule is designed to achieve RNA interference (or RNAi). In certain embodiments, the RNA molecule can be an anti-sense RNA, a small interfering RNA (siRNA), or a small hairpin RNA (shRNA). In certain embodiments, a mimic of a microRNA or an anti-microRNA can be transcribed from a DNA sequence of interest.

In certain embodiments, the RNA molecule is a self-replicating RNA (sr-RNA). Exemplary sr-RNAs are disclosed in Aliahmad et al., Cancer Gene Ther 2022 Feb. 22; 1-9, which is incorporated by reference herein in its entirety.

In certain embodiments, the sequence of interest comprises a nucleotide sequence encoding an mRNA for in-vitro transcription (IVT). In certain embodiments, the sequence of interest comprises a posttranscriptional regulatory element. In certain embodiments, the sequence of interest further comprises a polyadenylation and/or termination signal. In certain embodiments, the poly-adenylation is directly encoded in the sequence of interest such that the mRNA is directly synthesized with a polyadenylation sequence.

In some embodiments, the polyadenylation sequence is a homopolymeric sequence comprising an uninterrupted polyA sequence. In certain embodiments, the homopolymeric sequence is between 30-200 nucleotides in length. In certain embodiments, the homopolymeric sequence is at least 200 nucleotides in length. In some embodiments, the polyadenylation sequence is a homopolymeric sequence comprising two or more polyA sequences interrupted by at least one non-polyA sequence. In certain embodiments, each segment of polyA sequences is between 30-100 nucleotides in length. In certain embodiments, the segment of non-polyA sequences is between 1-15 nucleotides in length.

Size and location of the sequence of the RNA sequence of interest can be as described above.

### (iv) AAV Vectors

In certain embodiments, the sequence of interest comprises an expression cassette described in Section 5.1.1(c) flanked by 5′ and 3′ AAV ITRs described in Section 5.1.1(a) such that the hairpin-ended DNA is suitable as an AAV vector. In certain embodiments, the sequence of interest comprises a transcription unit described in Section 5.1.1(c) flanked by 5′ and 3′ AAV ITRs described in Section 5.1.1(a) such that the hairpin-ended DNA is suitable as an AAV vector. In certain embodiments, the sequence of interest encodes a promoter operably linked to an ORF flanked by AAV ITRs described in Section 5.1.1(a) such that the hairpin-ended DNA is suitable as an AAV vector. In certain embodiments, the sequence of interest further comprises other elements known in the art from AAV vectors, including but not limited to: posttranscriptional regulatory elements, polyadenylation and/or termination signals, 5′UTRs and/or 3′UTRs, and introns. In certain embodiments, these other elements may be located 3′ of the 5′ AAV ITR. In certain embodiments, the promoter is operably linked to a multicistronic or bicistronic sequence for the co-expression of multiple genes from a single transcript. In certain embodiments, the multicistronic or bicistronic sequence comprises IRES and/or 2A peptide between each gene. In certain embodiments, the sequence of interest encodes viral packaging and/or replication genes that include, but are not limited to Rep, Cap, and helper plasmids.

### (v) Lentivirus Vectors

In certain embodiments, the sequence of interest comprises an expression cassette described in Section 5.1.1(c) flanked by 5′ and 3′ LTRs such that the hairpin-ended DNA is suitable as a lentivirus transfer vector. In certain embodiments, the sequence of interest comprises a transcription unit described in Section 5.1.1(c) flanked by 5′ and 3′ LTRs such that the hairpin-ended DNA is suitable as a lentivirus transfer vector. In certain embodiments, the sequence of interest encodes a promoter operably linked to an ORF flanked by 5′ and 3′ LTRs such that the hairpin-ended DNA is suitable as a lentivirus transfer vector. In certain embodiments, the LTRs are derived from a known lentivirus. In certain embodiments, both LTRs are modified from wild-type LTR sequences. In certain embodiments, the 5′ LTR is a hybrid sequence, wherein said 5′ LTR is modified, optionally by replacing all or part of the U3 region with a heterologous promoter. In certain embodiments, the 3′ LTR is also modified, such that the LVVs produced are self-inactivating (SIN). In certain embodiments, the sequence of interest further comprises other elements known in the art from a lentivirus transfer vector, including but not limited to: the Psi packaging signal, the Rev response element (RRE) and/or the central polypurine tract (cPPT). In certain embodiments, these other elements may be located 3′ of the 5′ LTR. In certain embodiments, the promoter is operably linked to a multicistronic or bicistronic sequence for the co-expression of multiple ORF and/or ncRNAs from a single transcript. In certain embodiments, the multicistronic or bicistronic sequence comprises IRES between each ORF. In certain embodiments, a polyA signal is located downstream of the 3′ LTR. In certain embodiments, the sequence of interest encodes viral packaging genes that include, but are not limited to: Gag, Pol, Rev and/or Tat. In certain embodiments, the Gag gene and Pol gene may be encoded by a single hairpin-ended DNA molecule. In certain embodiments, the sequence of interest encodes one or more viral envelope gene from other viruses to improve the stability of the viral particle and confer either a broad tissue tropism, or specificity for target cells to Lentivirus vectors. In certain embodiments, the sequence of interest encodes a viral envelope gene comprising Vesicular Stomatitis Virus Glycoprotein (VSV-G).

### (vi) Additional Sequences

In certain embodiments, the sequence of interest does not encode a functional RNA or protein.

In certain embodiments, the sequence of interest comprises a gene promoter. In certain embodiments, the gene promoter is selected from the one disclosed in Section 5.1.1(c)(i). In certain embodiments, the sequence of interest comprises a T7 promoter.

In certain embodiments, the sequence of interest comprises an AAV ITR. In certain embodiments, the sequence of interest comprises at least one nucleotide sequence encoding an AAV ITR.

In certain embodiments, the sequence of interest comprises a synthetic DNA template to be integrated into a genome by a gene engineering technique (e.g., CRISPR/Cas system, transposase, prime editing).

### (d) Exemplary Hairpin-Ended Molecules

Exemplary hairpin-ended molecules made by the methods disclosed herein (see Sections 5.2-5.4.2) include the hairpin-ended DNA molecules disclosed in International Patent Publication No. WO 2022/023284, the content of which is incorporated by reference herein.

The hairpin-ended DNA molecules produced by the methods disclosed herein can comprise the inverted repeats (e.g., IRs and ITRs) that can form hairpins (e.g., hairpins disclosed in Section 5.1.1(a) and Section 5.1.1(d)), specific sequences, origins, and identities of IRs or ITRs as described in Sections 5.1.1(a) and 5.1.1(d), sequence of interest as described in 5.1.1(c), restriction sites for nicking endonucleases as described in Sections 5.1.1(b) and 5.3.2, and the targeting sites for programmable nicking enzymes as described in Section 5.3.2, and/or lacks the RABS and/or TRS sequences as described in Section 5.1.1(a).

The ITRs or the hairpinned ITRs in the hairpin-ended DNA molecules can be formed from the ITRs or IRs provided above in Sections 3 and 5.1.1(a), for example upon performing the method steps described in Sections 3 and 5.3.2-5.3.4. Accordingly, in certain embodiments, the two ITRs or the two hairpinned ITRs in the hairpin-ended DNA molecules disclosed herein can comprise any embodiments of the IRs or ITRs provided in Sections 3 and 5.1.1(a) and additional embodiments provided in Section 5.1.1(d), in any combination.

In one aspect, provided herein is a double strand DNA molecule comprising in 5′ to 3′ direction of the top strand: a) a first hairpinned inverted repeat (e.g., as described in Sections 5.1.1(a) and 5.1.1(d)); b) a nick of the bottom strand (e.g., as described in Sections 5.3.2, 5.1.1(b), and 5.1.1(d)); c) a sequence of interest (e.g., as described Sections 5.1.1(c) and 5.1.1(d)); d) a nick of the bottom strand (e.g., as described in Sections 5.3.2, 5.1.1(b), and 5.1.1(d)); and e) a second hairpinned inverted repeat (e.g., as described in Sections 5.1.1(a) and 5.1.1(d)).

In another aspect, provided herein is a double strand DNA molecule comprising in 5′ to 3′ direction of the top strand: a) a first hairpinned inverted repeat (e.g., as described in Sections 5.1.1(a) and 5.1.1(d)); b) a nick of the top strand (e.g., as described in Sections 5.3.2, 5.1.1(b), and 5.1.1(d)); c) a sequence of interest (e.g., as described Sections 5.1.1(c) and 5.1.1(d)); d.) a nick of the top strand (e.g., as described in Sections 5.3.2, 5.1.1(b), and 5.1.1(d)); and e) a second hairpinned inverted repeat (e.g., as described in Sections 5.1.1(a) and 5.1.1(d)).

In another aspect, provided herein is a double strand DNA molecule comprising in 5′ to 3′ direction of the top strand: a) a first hairpinned inverted repeat (e.g., as described in Sections 5.1.1(a) and 5.1.1(d)); b) a nick of the bottom strand (e.g., as described in Sections 5.3.2, 5.1.1(b), and 5.1.1(d)); c) a sequence of interest (e.g., as described Sections 5.1.1(c) and 5.1.1(d)); d.) a nick of the top strand (e.g., as described in Sections 5.3.2, 5.1.1(b), and 5.1.1(d)); and e) a second hairpinned inverted repeat (e.g., as described in Sections 5.1.1(a) and 5.1.1(d)).

In another aspect, provided herein is a double strand DNA molecule comprising in 5′ to 3′ direction of the top strand: a) a first hairpinned inverted repeat (e.g., as described in Sections 5.1.1(a) and 5.1.1(d)); b) a nick of the top strand (e.g., as described in Sections 5.3.2, 5.1.1(b), and 5.1.1(d)); c) a sequence of interest (e.g., as described Sections 5.1.1(c) and 5.1.1(d)); d.) a nick of the bottom strand (e.g., as described in Sections 5.3.2, 5.1.1(b), and 5.1.1(d)); and e) a second hairpinned inverted repeat (e.g., as described in Sections 5.1.1(a) and 5.1.1(d)).

The secondary structure is formed based on conformations (e.g., domains) that include base pair stacking, stems, hairpins, bulges, internal loops and multi-branch loops. A domain-level description of IRs represents the strand and formed complexes in terms of domains rather than specific nucleotide sequences. At the sequence level, each domain is assigned a particular nucleotide sequence or motif, and its complement's sequence is determined by Watson-Crick base pairing. This spans the full range of binding between any pair of complementary nucleotides, including G-T wobble base pairs. The overall set of bound (e.g., base paired) and unbound domains form a unimolecular complex and exhibit various secondary structures. In certain embodiments, hairpins can have a base-paired stem and a small loop of unpaired bases. In certain embodiments, the presence of interweaved non-palindromic polynucleotides sections in the polynucleotide sequence can lead to unpaired nucleotides known as bulges. Bulges can have one or more nucleotides and are classified in different types depending on their location: in the top strand (bulge), in both strands (internal loop), or at a junction. The collection of these base pairs constitutes the secondary structure of DNA which occurs in its three-dimensional structure.

A domain-level description for the DNA molecules provided herein are also provided to represent multiple strands and their complexes in terms of domains rather than specific nucleotide sequences. In certain embodiments, domains (e.g., sequences motifs) of interacting single stranded DNA strands can exhibit particular secondary structures on a single strand level that can interact with other DNA strands and, in some cases, take on a hybridized structure when a first strand is bound to a complementary domain on a second strand to form a duplex. Interactions of different DNA strands that generate new complexes or changes in secondary structure can be viewed as “reactions.” Additional unimolecular and bimolecular reactions are also possible at the sequence level. Poor sequence design can lead to sequence-level structures or interactions (e.g., multiple domains of complimentary in the expression cassette) that interfere with the intended reactions of a system comprising one or more DNA molecules provided herein. Undesired interactions can be avoided by design, resulting in reliable and predictable secondary structure formation.

The present disclosure provides that the underlying forces leading to the secondary structure of DNA are governed by hydrophobic interactions that underlie thermodynamic laws and the overall conformation may be influenced by physicochemical conditions. An exemplary list of factors determining equilibrium state include the type of solvent, chemical agents crowding, salt concentrations, pH and temperature. While free energy change parameters and enthalpy change parameters derived from experimental literature allow for a prediction of conformation stability, the overall three-dimensional structures of the hairpin formed from the IR sequences, as usual in statistical mechanics, corresponds to an ensemble of molecular conformations, not just one conformation. Predominant conformations can transition as the physical or chemical conditions (e.g., salts, pH or temperature) are permutated.

“Stem domain” or “stem” refers to a self-complementary nucleotide sequence of the overhang strand that will form Watson-Crick base pairs. The stem comprises primarily Watson-Crick base pairs formed between the two antiparallel stretches of DNA pairs and can be a right-handed helix. In certain embodiments, the stem comprises the stretch of self-complimentary DNA sequence in a palindromic sequence.

“Primary stem domain” or “primary stem” refers to the part of self-complementary or reverse complement nucleotide sequences of the ITR that is most proximal to the expression cassette or the non-ITR sequences of the DNA molecule. In certain embodiments, the primary stem domain is the self-complimentary stretch of a palindromic sequence that forms the termini of the DNA molecules provided herein and is covalently linked to the non-ITR sequences flanked by the ITRs. The primary stem encompasses both the start as well as the end of an IR sequence. In certain embodiments, the primary stems range in length from 1 to 100 or more base pairs (bp). The lengths of primary stem regions have an effect on denature/renature kinetics. In certain embodiments, the primary stem region can have at least approximately between 4 and 25 nucleotides to ensure thermal stability. In certain embodiments, the primary stem region can have between about 4 and 25 nucleotides to ensure thermal stability. On the other hand, the inverted repeat domains may be of any length sufficient to maintain an approximate three dimensional structure at physiological conditions.

“Loop” or “loop domain” refers to the region of unpaired nucleotides in an IR or ITR that is not a turning point and not in a stem. In certain embodiments, a loop domain is found at the apex of the IR structure. The loop domain can serve as the region in which the local directionality of the DNA strand is reversed to afford the two antiparallel strands of the originating stem. Because of steric repulsion, in certain embodiments, a loop comprises a minimum of two nucleotides to make a turn in a DNA hairpin. In certain embodiments, a loop comprises four nucleotides or more. In certain embodiments, a loop comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides. In some certain embodiments, a loop comprises about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 nucleotides. The loop follows a self-complementary sequence of a stem and serves to connect the further nucleotides to the stem domain. In certain embodiments, a loop can comprise a sequence of oligonucleotides that does not form a contiguous duplex structure with other nucleotides in the loop sequence or other elements of the ITR (e.g., the loop remains in flexible, single-stranded form). In certain embodiments, the loop sequence that does not form a duplex with other nucleotides in the loop sequence is a series of identical bases (e.g., AAAAAAAA (SEQ ID NO:321), CCCCCCCC (SEQ ID NO:322), GGGGGGG (SEQ ID NO: 323) or TTTTTTTT (SEQ ID NO:324)). In certain embodiments, the loop contains between 2 and 30 nucleotides. In certain embodiments, the loop domain contains between 2 and 15 nucleotides. In certain embodiments, the loop comprises a mixture of nucleotides.

As used herein, the term “hairpin” refers to any DNA structure as well as the overall DNA structure, including secondary or tertiary structure, formed from an IR or ITR sequence. As used herein, a “hairpinned” DNA molecule refers to a DNA molecule wherein one or more hairpins has formed in the DNA molecule. In certain embodiments, a hairpin comprises a complementary stem and a loop. A hairpin in its simplest form consists of a complementary stem and a loop. A structure encompassing stems and loops are referred to as “stem-loop,” “stem loop,” or “SL.” In certain embodiments, a hairpin consists of a complementary stem and a loop. “Branched hairpin” refers to a subset of hairpin that has multiple stem-loops that form branch structures. An IR or ITR after forming hairpin can be referred to as hairpinned ITR or IR. A “hairpin-ended” DNA molecule refers to a DNA molecule wherein a hairpin has formed at one end of the DNA molecule or a hairpin has formed at each of the 2 end of the DNA molecule.

“Turning point” or “apex” refers to the region of unpaired nucleotides at the spatial end of the ITR. The turning point serves as the region in which the global directionality of the DNA strand is reversed to afford the two antiparallel strands of the originating stem. The turning point also marks the point at which the IR or ITR sequence becomes inverted or the reverse compliment.

In certain embodiments, the part of ITR following the primary stem domain can encode a nucleotide sequence, which in contrast to regular double-stranded DNA, can form non-Watson-Crick-based structural elements when folding on itself, including wobbles and mismatches, and structural defects or imperfections, such as bulges and internal loops. A “bulge” contains one or more unpaired nucleotides on one strand, whereas “internal loops” contain one or more unpaired nucleotides on both top and bottom strands. Symmetric internal loops tend to distort the helix less than bulges and asymmetric internal loops, which can kink or bend the helix. In certain embodiments, the unpaired nucleotides in a stem can engage in diverse structural interactions, such as noncanonical hydrogen bonding and stacking, which lend themselves to additional thermodynamic stability and functional diversity.

In certain embodiments, a hairpin for the hairpin-ended DNA molecule comprises a primary stem. In certain embodiments, a hairpin for the hairpin-ended DNA molecule comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 stems. In certain embodiments, a hairpin for the hairpin-ended DNA molecule comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 loops. In certain embodiments, a hairpin for the hairpin-ended DNA molecule comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 internal loops. In certain embodiments, a hairpin for the hairpin-ended DNA molecule comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bulges. In certain embodiments, a hairpin for the hairpin-ended DNA molecule comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 branched hairpins. In certain embodiments, a hairpin for the hairpin-ended DNA molecule comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 apexes. In a certain embodiments, a hairpin for the hairpin-ended DNA molecule comprise any number of stems, branched hairpins, loops, bulges, apexes, and/or internal loops, in any combination.

In certain embodiments, the hairpin structure in the DNA molecules provided herein is formed by a symmetrical overhang. In order to obtain a symmetrical overhang, the modification in the 5′ stem region will require a cognate 3′ modification at the corresponding position in the stem region so that the modified 5′ position(s) can form base pair(s) with the modified 3′ position(s). Such modification to form a symmetrical overhang can be performed as described in the present disclosure in combination with the state of the art at the time of filing. For example, by generating a BstNBI restriction site for nicking endonuclease by an insertion of an A at position 23 will require an insertion of T at position 105 with respect to the wt AAV2 ITR (e.g.,

TTGGCCACTCCCTCTCTGCGCGACTCGCTCGCTCACTGAGGCCGGGCGACCAAAGGTCGCCCGA CGCCCGGGCTTTGCCCGGGCGGCCTCAGTGAGCGAGCGAGTCGCGCAGAGAGGGAGTGGCCAA (SEQ ID NO:162)).

In certain embodiments, the 5′ and 3′ hairpinned ITRs from a hairpinned ITR pair can have different reverse complement nucleotide sequences to harbor the antiparallel restriction sites for nicking endonuclease (e.g., 5′ ITR such that nicking results in a bottom strand 5′ overhang and the 3′ ITR such that nicking results in a bottom strand 3′ overhang) but still have the same three-dimensional spatial organization such that both ITRs have mutations that result in the same overall 3D shape.

In certain embodiments, hairpinned ITRs for use herein can comprise a modification (e.g., deletion, substitution or addition) of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in any one or more of the regions selected from: the primary stem domain, a stem, a branched hairpin, a loop, a bulge or an internal loop. In certain embodiments, the nucleotide in a right hairpinned ITR can be substituted from an A to a G, C or T or deleted or one or more nucleotides added; a nucleotide in a left hairpinned ITR can be changed from a T to a G, C or A, or deleted or one or more nucleotides added.

In certain embodiments, hairpinned ITRs for use herein can comprise a modification (e.g., deletion, substitution or addition) of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in any one or more of the regions selected from a primary stem domain, a stem, a branched hairpin, a loop, a bulge or an internal loop, in order to replace or deplete the occurrence of CpG motifs, thereby: (i) reducing or eliminating the binding of such modified hairpinned ITRs to toll like family of receptors (TLRs) (e.g., TLR9) compared to viral wild type ITRs, and/or (ii) reducing or diminishing ITR transcriptional activity by removing transcriptionally active CpG islands. Transcriptionally active CpG islands are commonly defined as sequences with a C+G ratio of greater than 50% and observed-to-expected CpG dinucleotides at 60% or higher as described in Gardiner-Garden M, Frommer M. CpG Islands in vertebrate genomes. J Mol Biol 1987; 196:261-282. In certain embodiments, the nucleotide in a right hairpinned ITR can be substituted from an Gor C to a A or T or deleted or one or more nucleotides added between a C and G or a G and C. In certain embodiments, a nucleotide in a left hairpinned ITR can be changed from a C or G to a T or A, or deleted or one or more nucleotides added between a C and G or a G and C. In certain embodiments the hairpinned ITRs comprise a CpG depleted sequence of TTGGTCACTCCCTCTCTGTACACTCACTCACTCACTGATCCCTGGATACCAAAGGTATCCAGACA CCCAGTCTTTGACTGGGTGGGATCAGTGAGTGAGTGAGTGTACAGAGAGGGAGTGACCAA (SEQ ID NO:325).

In certain embodiments, the hairpinned ITR of the DNA molecules provided herein can comprise a primary stem wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more complementary base pairs are removed from each of the primary stem domains such that the primary stem domain is shorter and has a lower free energy of folding. Briefly, in such embodiments, if a base is removed in the portion of the primary stem domain, the complementary base pair in the primary stem domain is also removed, thereby shortening the overall primary stem domain.

In certain embodiments, the hairpinned ITR of the DNA molecules provided herein can comprise a primary stem wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more complementary base pairs are introduced from each of the primary stem domains such that the primary stem domain is longer and has a higher free energy of folding. Briefly, in such embodiments, if a base is introduced in the portion of the primary stem domain, the complementary base pair in the primary stem domain is also introduced, thereby lengthening the overall primary stem domain.

In certain embodiments, the hairpinned ITR of the DNA molecules provided herein can comprise a primary stem wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more complementary base pairs are substituted from A or T to G or C from each of the primary stem domains such that the primary stem domain is more G/C rich and has a higher free energy of folding. Briefly, in such embodiments, if a base is substituted (e.g., T to G) in the portion of the primary stem domain, the complementary base pair in the primary stem domain is also substituted (e.g., A to C), thereby increasing the overall G/C content in the primary stem domain.

In certain embodiments, the hairpinned ITR of the DNA molecules provided herein can comprise a primary stem wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more complementary base pairs are substituted from G or C to A or T, or deleted or one or more nucleotides added between a C and G or a G and C, from each of the primary stem domains such that the primary stem domain contains less or no CpG motifs and has a lower TLR9 binding propensity than a viral ITR and/or fewer transcriptionally active CpG islands compared to a reference DNA (e.g., the same DNA molecule but with a unmodified primary stem sequence comprising CpG motifs).

In certain embodiments, the hairpinned ITR of the DNA molecules provided herein can comprise a primary stem wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more complementary base pairs are substituted from G or C to A or T from each of the primary stem domains such that RAPs (e.g., Rep) can no longer efficiently bind to the primary stem domain.

In certain embodiments, the hairpinned ITR of the DNA molecules provided herein can comprise a primary stem wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more complementary base pairs are substituted from A or T to G or C from each of the primary stem domains such that the primary stem domain is more G/C rich and has a higher free energy of folding such that RAPs (e.g., Rep or NS1) can no longer efficiently bind to the primary stem domain.

In certain embodiments, a hairpinned ITR sequence in the DNA molecules provided herein can have between 1 and 40 nucleotide deletions relative to a full-length wild-type (wt) viral ITR sequence while the whole wt ITR sequence is still present in the vector. For example, in a symmetric ITR such as the AAV2 ITR, if restriction sites for nicking endonuclease are each 25 bases away from the Apex, the portion after the restriction site for nicking endonuclease of the overhang does not need to be the wt IR sequence as it will be removed from the DNA molecules after incubation with nicking endonuclease (or nicking endonuclease and restriction enzymes) and denatured as described in Sections 5.3.3 and 5.3.2. In certain embodiments, a hairpinned ITR sequence in the DNA molecules provided herein can have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotide deletions relative to a full-length wt viral ITR sequence while the whole wt ITR sequence is still present in the vector.

In certain embodiments, the restriction site for nicking endonuclease is chosen based on the predicted melting temperature of the isolated nucleotide sequence present in the ITR stem region. In certain embodiments, the predicted melting temperature is between 40° C.-95° C. Certain embodiments are for the restriction site for nicking endonuclease and the embodiments factoring in melting temperature are described in Sections 5.3.2-5.3.4 and 5.1.1(b).

In certain embodiments, the length and GC content of the nucleotide sequence encompassing stem region of a hairpinned ITR in a DNA molecule provided herein is further modified by a deletion, insertion, and/or substitution so that a hairpin forms when the temperature is maintained at approximately 4° C. For example, the nucleotide sequence of the structural element can be modified as compared to the wild-type sequence of a viral ITR. In certain embodiments, the length and GC content of the stem is designed so that a hairpin forms when the temperature is maintained at approximately 10° C. or more below the melting temperature of the total ITR. The hairpin's melting temperature can be designed by changing the GC content, the distance between restriction sites for nicking endonuclease and the junction closest to the primary stem, or sequence mismatch or loop, so that the melting temperature is high enough to allow the hairpinned ITR to remain folded above 50° C. to ensure stable storage. The actual optimal length of the stem can vary with the sequence of the ITR and micro domains such as branches, loops and arms of the ITR, which can be determined according to the present disclosure in combination of the state of the art.

In certain embodiments, the stem region of the hairpinned ITR encode a restriction site for Class II nicking endonuclease (e.g., NNNN (SEQ ID NO: 326) downstream of 5′). In certain embodiments, the stem region does not contain a restriction site for Class II nicking endonuclease.

In certain embodiments, the stem region of the hairpinned ITR encode a restriction site for Class I nicking endonuclease. In certain embodiments, the stem region of the hairpinned ITR encode a restriction site for Class III, IV or V nicking endonuclease.

In certain embodiments, the sequence of interest in the hairpin-ended DNA molecules can be any embodiments of the expression cassette described in Section 5.1.1(c). In certain embodiments, the ITRs in the hairpin-ended DNA molecules can be any embodiments of the IR or ITR described in Section 5.1.1(a). In certain embodiments, the arrangement among the ITR, the expression cassette, and the restriction sites for nicking endonuclease or restriction enzymes can be any arrangement as described in Sections 5.3.2-5.3.4 and 5.1.1(a)-5.1.1(c).

In certain embodiments, the hairpin-ended DNA comprises a top strand that is covalently linked to the 3′ ITR as well as 5′ ITR and once the ITR is folded, the bottom strand is flanked by two nicks (a first and a second nick) at either end of the bottom strand such that the expression cassette is in between the first nick and the second nick, wherein the first nick is formed between the 3′ end of the bottom strand and the juxtaposed 5′ end of the top strand as a result of top strand 5′ ITR hairpin and the second nick is formed between the 5′ end of the bottom strand and the juxtaposed 3′ end of the top strand as a result of top strand 3′ ITR hairpin.

In certain embodiments, the hairpin-ended DNA comprises a bottom strand that is covalently linked to the 3′ ITR as well as 5′ ITR and once the ITR is folded, the top strand is flanked by two nicks (a first nick and a second nick) at either end of the top strand such that the expression cassette is in between the first nick and the second nick, wherein the first nick is formed between the 5′ end of the top strand and the juxtaposed 3′ end of the bottom strand as a result of bottom strand 3′ ITR hairpin and the second nick is formed between the 3′ end of the top strand and the juxtaposed 5′ end of the bottom strand as a result of bottom strand 3′ ITR hairpin.

In certain embodiments, the hairpin-ended DNA comprises a top strand that is covalently linked to the 5′ ITR and the bottom strand is covalently linked to the 5′ ITR so that when the ITRs are folded, the first nick is formed adjacent to the bottom strand between the 3′ end of the bottom strand and the juxtaposed 5′ end of the top strand as a result of top strand 5′ ITR hairpin and the second nick is formed adjacent to the top strand between the 3′ end of the top strand and the juxtaposed 5′ end of the bottom strand as a result of bottom strand 5′ ITR hairpin, with the expression cassette being flanked by the first and second nicks.

In certain embodiments, the hairpin-ended DNA comprises a top strand that is covalently linked to the 3′ ITR and the bottom strand is covalently linked to the 3′ ITR so that when the ITRs are folded, the first nick is formed adjacent to the top strand between the 5′ end of the top strand and the juxtaposed 3′ end of the bottom strand as a result of bottom strand 3′ ITR hairpin and the second nick is formed adjacent to the bottom strand between the 5′ end of the bottom strand and the juxtaposed 3′ end of the top strand as a result of top strand 3′ ITR hairpin, with the expression cassette being flanked by the first and second nicks.

In certain embodiments, the hairpin-ended DNA comprising the two nicks as described in Section 5.1.1(d) and the preceding 4 paragraphs can be ligated to repair the nicks by forming a covalent bond between the two nucleotides flanking the nick. In certain embodiments, one of the two nicks described in Section 5.1.1(d) and the preceding 4 paragraphs can be ligated and repaired such that when denatured, the DNA molecule becomes a linear single stranded DNA molecule. In certain embodiments, the two nicks described in Section 5.1.1(d) and the preceding 4 paragraphs can be ligated and repaired such that when denatured, the DNA molecule becomes a circular single stranded DNA molecule.

In certain embodiments, the two flanking ITR pairs in the hairpin-ended DNA molecule comprise identical DNA sequence. In certain embodiments, the two flanking ITR pairs in the hairpin-ended DNA molecule comprise different DNA sequences. In certain embodiments, one of the ITRs in the hairpin-ended DNA molecule is modified by deletion, insertion, and/or substitution as compared to the other ITR in the same hairpin-ended DNA molecule. In certain embodiments, the first ITR and the second ITR in the hairpin-ended DNA molecule are both modified, e.g., by deletion, insertion, and/or substitution. In certain embodiments, the first ITR and the second ITR in the hairpin-ended DNA molecule comprise different DNA sequences and are both modified. In certain embodiments, the first ITR and the second ITR in the hairpin-ended DNA molecule comprise different DNA sequences and are both modified, wherein the modifications for the two ITRs are different. In certain embodiments, the first ITR and the second ITR in the hairpin-ended DNA molecule comprise different DNA sequences and are both modified, wherein the modifications for the two ITRs are identical. In certain embodiments, the first ITR and the second ITR in the hairpin-ended DNA molecule comprise identical DNA sequence and are both modified, wherein the modifications for the two ITRs are different. In certain embodiments, the first ITR and the second ITR in the hairpin-ended DNA molecule comprise identical DNA sequence and are both modified, wherein the modifications for the two ITRs are identical. In certain embodiments, the first ITR and the second ITR in the hairpin-ended DNA are both modified ITRs and the two modified ITRs are not identical. In certain embodiments, the hairpin-ended DNA molecules comprise two ITRs that are asymmetric, wherein the asymmetry can be a result of any changes in one ITR that are not reflected in the other ITR. In certain embodiments, the hairpin-ended DNA molecules comprise two ITRs that are asymmetric, wherein the ITRs are different with respect to each other in any way. In certain embodiments, the modifications provided in this paragraph, including deletion, insertion, and/or substitution, can be any such modifications described above in Section 5.1.1(d).

In certain embodiments a hairpin-ended DNA molecule provided herein comprises, in the 5′ to 3′ direction: a first IR, a sequence of interest (e.g., as described in Sections 5.1.1(c)) and a second IR.

In certain embodiments, a hairpin-ended DNA molecule provided herein comprises, in the 5′ to 3′ direction: a first IR (e.g. a 5′ ITR), a promoter, an ORF, a polyadenylation and/or termination signal, and a second IR (e.g. a 3′ ITR).

In certain embodiments, a hairpin-ended DNA molecule provided herein comprises, in the 5′ to 3′ direction of the top strand: a) a first hairpinned inverted repeat (e.g., a 5′ ITR); b) a nick of the bottom strand; c) a promoter, an ORF, and a polyadenylation and/or termination signal; d) a nick of the bottom strand; and e) a second hairpinned inverted repeat (e.g., 3′ ITR). In certain embodiments, a hairpin-ended DNA molecule provided herein comprises, in the 5′ to 3′ direction of the top strand: a) a first hairpinned inverted repeat (e.g., a 5′ ITR); b) a nick of the top strand; c) a promoter, an ORF, and a polyadenylation and/or termination signal; d) a nick of the top strand; and e) a second hairpinned inverted repeat (e.g., 3′ ITR). In certain embodiments, a hairpin-ended DNA molecule provided herein comprises, in the 5′ to 3′ direction of the top strand: a) a first hairpinned inverted repeat (e.g., a 5′ ITR); b) a nick of the bottom strand; c) a promoter, an ORF, and a polyadenylation and/or termination signal; d) a nick of the top strand; and e) a second hairpinned inverted repeat (e.g., 3′ ITR). In certain embodiments, a hairpin-ended DNA molecule provided herein comprises, in the 5′ to 3′ direction of the top strand: a) a first hairpinned inverted repeat (e.g., a 5′ ITR); b) a nick of the top strand; c) a promoter, an ORF, and a polyadenylation and/or termination signal; d) a nick of the bottom strand; and e) a second hairpinned inverted repeat (e.g., 3′ ITR).

In certain embodiments, a hairpin-ended DNA molecule provided herein comprises, in the 5′ to 3′ direction: a first IR (e.g. a 5′ ITR), a promoter, an ORF, a polyadenylation and/or termination signal, and a second IR (e.g. a 3′ ITR). In certain embodiments, a hairpin-ended DNA molecule provided herein comprises, in the 5′ to 3′ direction: a first IR (e.g. a 5′ ITR), a promoter, a UTR (e.g. a 5′ UTR), an ORF, a polyadenylation and/or termination signal, and a second IR (e.g. a 3′ ITR). In certain embodiments, a hairpin-ended DNA molecule provided herein comprises, in the 5′ to 3′ direction: a first IR (e.g. a 5′ ITR), a promoter, a UTR (e.g. a 5′ UTR), an ORF comprising two protein-encoding sequences operably linked by a self-cleaving peptide, a polyadenylation and/or termination signal, and a second IR (e.g. a 3′ ITR). In certain embodiments, a hairpin-ended DNA molecule provided herein comprises, in the 5′ to 3′ direction: a first IR (e.g. a 5′ ITR), a promoter, a UTR (e.g. a 5′ UTR), an ORF, a spacer, a polyadenylation and/or a termination signal, and a second IR (e.g. a 3′ ITR). In certain embodiments, a hairpin-ended DNA molecule provided herein comprises, in the 5′ to 3′ direction: a first IR (e.g. a 5′ ITR), a promoter, a UTR (e.g. a 5′ UTR), an ORF comprising two protein-encoding sequences operably linked by a self-cleaving peptide, a spacer, a polyadenylation and/or a termination signal, and a second IR (e.g. a 3′ ITR). In certain embodiments, a hairpin-ended DNA molecule provided herein comprises, in the 5′ to 3′ direction: a first IR (e.g. a 5′ ITR), a promoter, a UTR (e.g. a 5′ UTR), an ORF, a first spacer, a polyadenylation and/or a termination signal, a second spacer, and a second IR (e.g. a 3′ ITR). In certain embodiments, a hairpin-ended DNA molecule provided herein comprises, in the 5′ to 3′ direction: a first IR (e.g. a 5′ ITR), a promoter, a UTR (e.g. a 5′ UTR), an ORF comprising two protein-encoding sequences operably linked by a self-cleaving peptide, a first spacer, a polyadenylation and/or a termination signal, a second spacer, and a second IR (e.g. a 3′ ITR). In certain embodiments, a hairpin-ended DNA molecule provided herein comprises, in the 5′ to 3′ direction: a first IR (e.g. a 5′ ITR), a promoter, a UTR (e.g. a 5′ UTR), an ORF, a first spacer, a polyadenylation signal, a second spacer, and a second IR (e.g. a 3′ ITR). In certain embodiments, a hairpin-ended DNA molecule provided herein comprises, in the 5′ to 3′ direction: a first IR (e.g. a 5′ ITR), a promoter, a UTR (e.g. a 5′ UTR), an ORF comprising two protein-encoding sequences operably linked by a self-cleaving peptide, a first spacer, a polyadenylation signal, a second spacer, and a second IR (e.g. a 3′ ITR). In certain embodiments, a hairpin-ended DNA molecule provided herein comprises, in the 5′ to 3′ direction: a first IR (e.g. a 5′ ITR), a promoter, a UTR (e.g. a 5′ UTR), an ORF, a first spacer, a polyadenylation and a termination signal, a second spacer, and a second IR (e.g. a 3′ ITR). In certain embodiments, a hairpin-ended DNA molecule provided herein comprises, in the 5′ to 3′ direction: a first IR (e.g. a 5′ ITR), a promoter, a UTR (e.g. a 5′ UTR), an ORF comprising two protein-encoding sequences operably linked by a self-cleaving peptide, a first spacer, a polyadenylation and a termination signal, a second spacer, and a second IR (e.g. a 3′ ITR). In one embodiment, the ORF encodes a transgene product. In one embodiment, the ORF encodes an AAV vector genome, wherein the AAV vector genome comprises a 5′ ITR, an expression cassette, and a 3′ ITR.

In certain embodiments, a hairpin-ended DNA molecule provided herein comprises, in the 5′ to 3′ direction: a first IR (e.g. a 5′ ITR), a spacer, a promoter, an ORF, a polyadenylation and/or termination signal, and a second IR (e.g. a 3′ ITR). In certain embodiments, a hairpin-ended DNA molecule provided herein comprises, in the 5′ to 3′ direction: a first IR (e.g. a 5′ ITR), a spacer, a promoter, an ORF comprising two protein-encoding sequences operably linked by a self-cleaving peptide, a polyadenylation and/or termination signal, and a second IR (e.g. a 3′ ITR). In certain embodiments, a hairpin-ended DNA molecule provided herein comprises, in the 5′ to 3′ direction: a first IR (e.g. a 5′ ITR), a first spacer, a promoter, an ORF, a second space, a polyadenylation and/or termination signal, a third spacer, and a second IR (e.g. a 3′ ITR). In certain embodiments, a hairpin-ended DNA molecule provided herein comprises, in the 5′ to 3′ direction: a first IR (e.g. a 5′ ITR), a first spacer, a promoter, an ORF comprising two protein-encoding sequences operably linked by a self-cleaving peptide, a second space, a polyadenylation and/or termination signal, a third spacer, and a second IR (e.g. a 3′ ITR). In certain embodiments, a hairpin-ended DNA molecule provided herein comprises, in the 5′ to 3′ direction: a first IR (e.g. a 5′ ITR), a first spacer, a promoter, an ORF, a second space, a polyadenylation signal, a third spacer, and a second IR (e.g. a 3′ ITR). In certain embodiments, a hairpin-ended DNA molecule provided herein comprises, in the 5′ to 3′ direction: a first IR (e.g. a 5′ ITR), a first spacer, a promoter, an ORF comprising two protein-encoding sequences operably linked by a self-cleaving peptide, a second space, a polyadenylation signal, a third spacer, and a second IR (e.g. a 3′ ITR). In other embodiments, the promoter is a CMV-IE promoter. In certain embodiments, the self-cleaving peptide is a T2A self-cleaving peptide. In one embodiment, the ORF encodes a transgene product. In one embodiment, the ORF encodes an AAV vector genome, wherein the AAV vector genome comprises a 5′ ITR, an expression cassette, and a 3′ ITR.

In certain embodiments, a hairpin-ended DNA molecule provided herein comprises, in the 5′ to 3′ direction: a first IR (e.g. a 5′ ITR), a sequence of interest as described in Section 5.1.1(c), and a second IR (e.g. a 3′ ITR). In certain embodiments, a hairpin-ended DNA molecule provided herein comprises, in the 5′ to 3′ direction: a first IR (e.g. a 5′ ITR), a spacer, a sequence of interest as described in Section 5.1.1(c), and a second IR (e.g. a 3′ ITR). In certain embodiments, a hairpin-ended DNA molecule provided herein comprises, in the 5′ to 3′ direction: a first IR (e.g. a 5′ ITR), a first spacer, a sequence of interest as described in Section 5.1.1(c), a second spacer, and a second IR (e.g. a 3′ ITR). In one embodiment, the first IR (e.g. the 5′ ITR) is an AAV2 ITR. In one embodiment, the second IR (e.g. the 3′ ITR) is an AAV2 ITR. In one embodiment, at least one of the inverted repeats comprises a minimal required origin of replication. In one embodiment, at least one of the inverted repeats comprises at least one viral replication-associated protein binding sequence (“RABS”). In one embodiment, the first IR comprises the nucleotide sequence of SEQ ID NO: 529. In one embodiment, the second IR comprises the nucleotide sequence of SEQ ID NO: 530. In one embodiment, the sequence of interest encodes a recombinant AAV vector genome. In one embodiment, the sequence of interest comprises at least one ORF. In one embodiment, the sequence of interest comprises at least one ORF encoding a transgene product. In one embodiment, the sequence of interest encodes an AAV Rep protein and/or AAV capsid proteins. In one embodiment, the sequence of interest encodes an AAV Rep protein and/or AAV capsid proteins, and the first IR and second IR do not contain a RABS. In one embodiment, the sequence of interest encodes at least one component of a helper plasmid. In one embodiment, the sequence of interest encodes at least one component of a helper plasmid, and the first IR and second IR do not contain a RABS.

In certain embodiments, a hairpin-ended DNA molecule provided herein comprises, in the 5′ to 3′ direction: a first IR (e.g. a 5′ ITR), a 5′ LTR, an ORF, a 3′ LTR, and a second IR (e.g. a 3′ ITR). In certain embodiments, a hairpin-ended DNA molecule provided herein comprises, in the 5′ to 3′ direction: a first IR (e.g. a 5′ ITR), a 5′ LTR, a promoter, an ORF, a 3′ LTR, and a second IR (e.g. a 3′ ITR). In certain embodiments, a hairpin-ended DNA molecule provided herein comprises, in the 5′ to 3′ direction: a first IR (e.g. a 5′ ITR), a 5′ LTR, a promoter, an ORF, a 3′ LTR, a polyadenylation and/or termination signal, and a second IR (e.g. a 3′ ITR). In certain embodiments, a hairpin-ended DNA molecule provided herein comprises, in the 5′ to 3′ direction: a first IR (e.g. a 5′ ITR), a 5′ LTR, a promoter, an ORF, a 3′ LTR, a 3′ UTR, a polyadenylation and/or termination signal, and a second IR (e.g. a 3′ ITR). In certain embodiments, a hairpin-ended DNA molecule provided herein comprises, in the 5′ to 3′ direction: a first IR (e.g. a 5′ ITR), a spacer, a 5′ LTR, a promoter, an ORF, a 3′ LTR, a 3′ UTR, a polyadenylation and/or termination signal, and a second IR (e.g. a 3′ ITR). In certain embodiments, a hairpin-ended DNA molecule provided herein comprises, in the 5′ to 3′ direction: a first IR (e.g. a 5′ ITR), a first spacer, a 5′ LTR, a promoter, an ORF, a 3′ LTR, a 3′ UTR, a polyadenylation and/or termination signal, a second spacer, and a second IR (e.g. a 3′ ITR). In certain embodiments, a hairpin-ended DNA molecule provided herein comprises, in the 5′ to 3′ direction: a first IR (e.g. a 5′ ITR), a first spacer, a 5′ LTR, a promoter, an ORF, a IRES sequence, a 3′ LTR, a 3′ UTR, a polyadenylation and/or termination signal, a second spacer, and a second IR (e.g. a 3′ ITR). In certain embodiments, a hairpin-ended DNA molecule provided herein comprises, in the 5′ to 3′ direction: a first IR (e.g. a 5′ ITR), a first spacer, a first promoter, a 5′ LTR, a Psi sequence, a RRE sequence, a cPPT/CTS sequence, a second promoter, an ORF, a IRES sequence, a WPRE sequence, a 3′ LTR, a 3′ UTR, a polyadenylation and/or termination signal, a second spacer, and a second IR (e.g. a 3′ ITR). In certain embodiments, the ORF encodes a transgene product.

In certain embodiments, a hairpin-ended DNA molecule provided herein comprises, in the 5′ to 3′ direction: a first IR (e.g. a 5′ ITR), a promoter, an ORF, a 3′ UTR, a polyadenylation and/or termination signal, and a second IR (e.g. a 3′ ITR). In certain embodiments, a hairpin-ended DNA molecule provided herein comprises, in the 5′ to 3′ direction: a first IR (e.g. a 5′ ITR), a spacer, a promoter, an ORF, a 3′ UTR, a polyadenylation and/or termination signal, and a second IR (e.g. a 3′ ITR). In certain embodiments, a hairpin-ended DNA molecule provided herein comprises, in the 5′ to 3′ direction: a first IR (e.g. a 5′ ITR), a first spacer, a promoter, an ORF, a 3′ UTR, a polyadenylation and/or termination signal, a second spacer, and a second IR (e.g. a 3′ ITR). In certain embodiments, the ORF encodes a transgene product. In certain embodiments, the ORF encodes Gag and/or Pol proteins. In certain embodiments, the ORF encodes a VSV-G protein.

In certain embodiments, a hairpin-ended DNA molecule provided herein comprises, in the 5′ to 3′ direction: a first IR (e.g. a 5′ ITR), a promoter, a 5′ UTR, an ORF, a 3′ UTR, a polyadenylation and/or termination signal, and a second IR (e.g. a 3′ ITR). In certain embodiments, a hairpin-ended DNA molecule provided herein comprises, in the 5′ to 3′ direction: a first IR (e.g. a 5′ ITR), a spacer, a promoter, a 5′ UTR, an ORF, a 3′ UTR, a polyadenylation and/or termination signal, and a second IR (e.g. a 3′ ITR). In certain embodiments, a hairpin-ended DNA molecule provided herein comprises, in the 5′ to 3′ direction: a first IR (e.g. a 5′ ITR), a first spacer, a promoter, a 5′ UTR, an ORF, a 3′ UTR, a polyadenylation and/or termination signal, a second spacer, and a second IR (e.g. a 3′ ITR). In certain embodiments, the ORF encodes a transgene product. In certain embodiments, the ORF encodes a Rev protein. In certain embodiments, the ORF encodes Gag and/or Pol proteins. In certain embodiments, the ORF encodes VSV-G protein.

In certain embodiments, a hairpin-ended DNA molecule provided herein comprises, in the 5′ to 3′ direction: a first IR (e.g. a 5′ ITR), a promoter (suitable for IVT), an ORF, and a second IR (e.g. a 3′ ITR). In certain embodiments, a hairpin-ended DNA molecule provided herein comprises, in the 5′ to 3′ direction: a first IR (e.g. a 5′ ITR), a promoter (suitable for IVT), a UTR (e.g. a 5′ UTR), an ORF, and a second IR (e.g. a 3′ ITR). In certain embodiments, the hairpin-ended DNA molecule comprises a restriction site immediately downstream of the ORF. In certain embodiments, a hairpin-ended DNA molecule provided herein comprises, in the 5′ to 3′ direction: a first IR (e.g. a 5′ ITR), a promoter (suitable for IVT), a UTR (e.g. a 5′ UTR), an ORF, a polyadenylation sequence, and a second IR (e.g. a 3′ ITR). In certain embodiments, a hairpin-ended DNA molecule provided herein comprises, in the 5′ to 3′ direction: a first IR (e.g. a 5′ ITR), a promoter (suitable for IVT), a first UTR (e.g. a 5′ UTR), an ORF, a second UTR (e.g. a 3′ UTR), a polyadenylation sequence, and a second IR (e.g. a 3′ ITR). In certain embodiments, a hairpin-ended DNA molecule provided herein comprises, in the 5′ to 3′ direction: a first IR (e.g. a 5′ ITR), a promoter (suitable for IVT), a first UTR (e.g. a 5′ UTR), an ORF, a second UTR (e.g. a 3′ UTR), a polyadenylation sequence, a spacer, and a second IR (e.g. a 3′ ITR). In certain embodiments, a hairpin-ended DNA molecule provided herein comprises, in the 5′ to 3′ direction: a first IR (e.g. a 5′ ITR), a first spacer, a promoter (suitable for IVT), a first UTR (e.g. a 5′ UTR), an ORF, a second UTR (e.g. a 3′ UTR), a polyadenylation sequence, a second spacer, and a second IR (e.g. a 3′ ITR). In certain embodiments, the hairpin-ended DNA molecule comprises a restriction site immediately downstream of the polyadenylation sequence. In certain embodiments, the hairpin-ended DNA molecule comprises a restriction site upstream of the promoter.

The hairpin-ended DNA molecules can comprise a combination of dsDNA and ssDNA. In certain embodiments, certain portion of the hairpin-ended DNA molecules disclosed herein is dsDNA. In certain embodiments, the dsDNA portion of the hairpin-ended DNA molecules comprises the sequence of interest, a stem region of the ITR, or both. In certain embodiments, certain portion of the hairpin-ended DNA molecules is ssDNA. In certain embodiments, the dsDNA portion of the hairpin-ended DNA molecules accounts for over 90% of the hairpin-ended DNA molecules. In certain embodiments, the dsDNA portion of the hairpin-ended DNA molecules accounts for at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the hairpin-ended DNA molecules. In certain embodiments, the dsDNA portion of the hairpin-ended DNA molecules accounts for about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of the hairpin-ended DNA molecules.

In certain embodiments, the hairpin-ended DNA molecule provided herein can be efficiently targeted or transported to the nucleus of a cell. In certain embodiments, the hairpin-ended DNA molecule provided herein can be efficiently targeted or transported to the nucleus of a cell by the binding between the aptamer formed at the ITR and a nucleus protein. In certain embodiments, the hairpin-ended DNA molecule provided herein can be efficiently targeted or transported to the nucleus of a cell, such that the abundance of the hairpin-ended DNA molecules in the nucleus is 10%, 20%, 30%, 40%, 50%, 60%, 70%, 80%, 90%, or 100% higher than that in the cytoplasm. In certain embodiments, the hairpin-ended DNA molecule provided herein can be efficiently targeted or transported to the nucleus of a cell, such that the abundance of the hairpin-ended DNA molecules in the nucleus is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 fold higher than that in the cytoplasm.

In various embodiments of the hairpin-ended DNA molecules provided herein, the hairpin-ended DNA molecule lacks the RABS and/or TRS sequences as described in Section 5.1.5. In other embodiments of the hairpin-ended DNA molecule provided herein, the hairpin-ended DNA molecule lacks any or any combination of the DNA sequences, elements, or features as described in Section 5.1.5.

In certain embodiments, the hairpin-ended DNA molecules can be in any embodiment with respect to purity as described in Section 5.1.1(f).

### (e) Functional Properties of the Hairpin-Ended DNA Molecules

In certain embodiments, the ITR promotes the long-term survival of the nucleic acid molecule in the nucleus of a cell. In certain embodiments, the ITR promotes the permanent survival of the nucleic acid molecule in the nucleus of a cell (e.g., for the entire life-span of the cell). In certain embodiments, the ITR promotes the stability of the nucleic acid molecule in the nucleus of a cell. In certain embodiments, the ITR inhibits or prevents the degradation of the nucleic acid molecule in the nucleus of a cell.

In certain embodiments, when the ITR assumes its folded state, it is resistant to exonuclease digestion (e.g., exonuclease V), e.g., for over an hour at 37° C. In certain embodiments, the hairpin-ended DNA molecule is resistant to exonuclease digestion (e.g., digestion by exonuclease V). In certain embodiments, the hairpin-ended DNA molecule is resistant to exonuclease digestion (e.g., digestion by exonuclease V) for at least 1, at least 2, at least 3, at least 4, at least 5, at least 6, at least 7, at least 8, at least 9, at least 10 or more hours. In certain embodiments, the hairpin-ended DNA molecule is resistant to exonuclease digestion (e.g., digestion by exonuclease V) for about 1, about 2, about 3, about 4, about 5, about 6, about 7, about 8, about 9, or about 10 hours.

Hairpin-ended DNA molecules similar to viral ITRs can be produced without the need for RAPs and consequently independent of the RABS or TRS sequence for genome replication. Accordingly, the RABS and TRS can optionally be encoded in the nucleotide sequence disclosed herein but are not required and offer flexibility with regard to designing the ITRs. In certain embodiments, the DNA molecules provided herein comprise ITRs that do not comprise RABS. In certain embodiments, the DNA molecules provided herein comprise ITRs that do not comprise TRS. In certain embodiments, the DNA molecules provided herein comprise ITRs that do not comprise either RABS or TRS. In certain embodiments, the DNA molecules provided herein comprise ITRs that comprise RABS, TRS, or both RABS and TRS.

In certain embodiments, the hairpin-ended DNA molecules provided herein are stable in the host cell. In certain embodiments, the hairpin-ended DNA molecules provided herein are stable in the host cell for long term culture. In certain embodiments, the hairpin-ended DNA molecules provided herein can be efficiently delivered to a host cell.

The DNA molecules provided herein have superior stability, not just for their resistance to exonuclease digestion described above, but also with respect to their structure. In certain embodiments, the structure of the DNA molecules remains the same after storage at room temperature for 1 day, 2 days, 3 days, 4 days, 5 days, 6 days, 7 days, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, or 12 months. In certain embodiments, the ensemble structure of the DNA molecules remains the same after storage at room temperature for 1 day, 2 days, 3 days, 4 days, 5 days, 6 days, 7 days, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, or 12 months. In certain embodiments, the structure of the DNA molecules provided herein is the same after 2, 3, 4, 5, 10 or 20 cycles of denaturing/renaturing (e.g., denaturing as described in Section 5.3.3 and re-annealing as described in Section 5.3.4). DNA structures can be described by an ensemble of structures at or around the energy minimum. In certain embodiments, the ensemble DNA structure is the same after 2, 3, 4, 5, 10 or 20 cycles of denaturing/renaturing. In certain embodiments, the folded hairpin structure formed from the ITR or IR provided herein is the same after 2, 3, 4, 5, 10 or 20 cycles of denaturing/renaturing. In certain embodiments, the ensemble structure of the folded hairpin is the same after 2, 3, 4, 5, 10 or 20 cycles of denaturing/renaturing.

### (f) Compositions Comprising Hairpin-Ended DNA Molecules

One advantage of the methods disclosed herein is that the produced hairpin-ended DNA molecules are resistant to exonuclease or other DNA digestion enzymes digestion as described in Section 5.4.2, and thus the undesirable DNA molecules and DNA contaminants that are susceptible to such treatment can be removed from the compositions and reaction mixtures. Therefore, compositions produced by the methods disclosed herein comprise a high purity hairpin-ended DNA molecules of interest (i.e., hairpin-ended DNA molecules comprising a sequence of interest), and thus the hairpin-ended DNA molecules and compositions comprising thereof are transfection ready and translation ready (e.g., the sequence of interest encodes an in vitro transcribed (IVT) mRNA). Compositions disclosed herein can have various purity of hairpin-ended DNA molecules of interest. Furthermore, a person of ordinary skill in the art would understand that the compositions disclosed herein can be free of certain general DNA contaminants, free of certain specific DNA contaminants, or free of both.

Accordingly, in certain embodiments, the compositions disclosed herein are free of undesired DNA molecules. In certain embodiments, the undesired DNA molecules comprise hairpin-ended or non-hairpin-ended DNA molecules that do not comprise the sequence of interest.

In certain embodiments, the composition disclosed herein comprises the undesired DNA molecules at a level of no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, or no more than 50% of the hairpin-ended DNA molecules. In certain embodiments, the composition disclosed herein comprises the undesired DNA molecules at a level of less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, or less than 50% of the hairpin-ended DNA molecules. In certain embodiments, the composition disclosed herein comprises the undesired DNA molecules at a level of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% of the hairpin-ended DNA molecules.

### 5.1.2 Primer-binding Sites for Amplification

In certain embodiments, methods disclosed herein comprise incubating the DNA templates with polymerase and primers for amplification of the DNA template ((e.g., isothermal amplification, e.g., RCA, MDA)) (see Section 5.2). In certain embodiments, site-specific primers can be used with the methods and compositions provided herein. Random primers can also be used (such as random hexamers).

In certain embodiments, DNA molecules disclosed herein (e.g., DNA templates, amplification products) comprise site-specific primer binding sites. In certain embodiments, site-specific primers used with the methods disclosed herein comprise a primer pair. One primer in such a primer pair is complementary to and thus hybridizes to one strand of the DNA template thereby initiating synthesis of the complementary strand of DNA; the other primer is complementary to and thus hybridizes to that complementary strand of DNA thereby initiating the synthesis of the second strand of DNA resulting in the synthesis of double-stranded DNA (e.g., amplification products disclosed herein). The template can be single-stranded or double-stranded. In certain embodiments, two or more (e.g., 2, 3, 4, 5, 6, 7, 8, 9, 10, or more) site-specific primers can be used for the synthesis of the first strand and/or for the synthesis of the second strand. The sections below describe the primer-binding sites that can be used with the methods and compositions provided herein primarily for a primer pair of a first primer and a second primer that are designed to result in the synthesis of double stranded DNA (e.g., amplification products disclosed herein) from either a single stranded or a double stranded template. The skilled artisan would know, however, based in this guidance how to design primers and primer-binding sites for such methods if more than a primer pair of two primers is being used.

### (a) Size

The size of both primer binding sites in a primer pair can be, independently from each other, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 60, 65, 70, 75, 80, 85, 90, 95, 100 or more nucleotides long to allow production of an amplification product. In certain embodiments, the size of both primer binding sites in a primer pair is, independently from each other up to 10, up to 25, up to 30, up to 35, up to 40, up to 45, up to 50, up to 55, up to 60, up to 65, up to 70, up to 75, up to 75, up to 80, up to 85, up to 90, up to 95, or up to 100 nucleotides long. In certain embodiments, the size of both primer binding sites in a primer pair is, independently from each other up to 100 nucleotides long.

In certain embodiments, the DNA polymerase is a DNA polymerase mutant (e.g., a DNA polymerase mutant disclosed in WO2020234200, the content of which is incorporated by reference in its entirety), which can recognize primers that have less than 5 nucleotides long (e.g., 3 nucleotides long). In certain embodiments, the size of both primer binding sites in a primer pair is, independently from each other less than 5 nucleotides long (e.g., 3 nucleotides long).

In certain embodiments, both primer binding sites have about, i.e., +/−10%, the same length. In certain embodiments, both primer sites have the same length.

### (b) G/C Content

In certain embodiments, the GC content of the first and the second primer binding sites are, independent from each other, at least 30%, at least 35%, at least 40%, at least 45%, at least 50%, at least 55%, at least 60%, at least 65%, at least 70%, at least 75%, or at least 80%. In certain embodiments, the GC content of the first and the second primer sites are, independent from each other, at most 30%, at most 35%, at most 40%, at most 45%, at most 50%, at most 55%, at most 60%, at most 65%, at most 70%, at most 75%, or at most 80%.

### (c) Sequence and Location

In certain embodiments, the sequences of the first and the second primers are, independent from each, at least 80%, at least 85%, at least 90%, at least 95%, or is 100% complementary to the template strand and thus to their respective primer binding sites.

In certain embodiments, the 3′ end of a primer and thus the 5′ end of the respective primer binding site to be used with the methods and compositions provided herein is a Guanosine (“G”) or a Cytosine (“C”), two G/Cs, or three G/Cs. In certain embodiments, the primers comprise other bases. Non-limiting examples of primers that can be used with the present disclosure are described in Section 5.2.2(b). Exemplary primers and methods of use thereof are demonstrated in Section 7.4.

In certain embodiments, the binding site of the first primer and/or the second primer is located outside the segment comprising the inverted repeat-flanked sequence of interest. In other words, the primer binding site is located on the opposite side of both inverted repeats relative to the sequence of interest. In certain embodiments, the primer binding site is not in the inverted repeat and/or is not in the sequence of interest. Due to N-1 impurity, having the primer binding site located outside the segment comprising the inverted repeat-flanked sequence of interest improves the purity of the amplified DNA products and results in controlled DNA amplification. In certain embodiments, the primer binding sites for the primer pair are flanking each inverted repeat in a way that the 3′ end of the first and the second primers point towards the inverted repeats and the sequence of interest. The distance between the 3′ end of the first and the second primers can vary at a wide range, such that the location of the first primer binding site is independent from the second primer binding site. The distance between the primer binding sites and the downstream restriction enzyme sites, MSRE sites, and MSNE sites, which are located outside the segment comprising the inverted repeat-flanked sequence of interest, can also vary at a wide range

In certain embodiments, the binding site of the first primer and/or the second primer is located inside the segment comprising the inverted repeat-flanked sequence of interest. In certain embodiments, the binding site is located in an ITR, backbone sequence, or the sequence of interest.

In certain embodiments, the binding site is not in the MSRE or MSNE site. In certain embodiments, the binding site is in the MSRE or MSNE site, and the primers are modified to allow protection of the formed double strand upon primer binding.

In certain embodiments, the binding sites for the first and second primers are at different locations in the DNA template. In certain embodiments, the binding sites for the first and second primers are not complementary to each other. In certain embodiments, the binding sites for the first and second primers are less than 50%, less than 40%, less than 30%, less than 20%, less than 10%, less than 5%, or less 1% complementary to each other.

In certain embodiments, the binding sites for the first and second primers are 100% complementary to each other.

### 5.1.3 Restriction Enzyme Sites

In certain embodiments, DNA templates disclosed herein and/or amplification products produced therefrom comprise a restriction enzyme site. Such a restriction enzyme site is not present in the hairpin-ended DNA molecules of interest (i.e., hairpin-ended DNA molecules comprising a sequence of interest). The inclusion of such a restriction enzyme site in the amplification products serves several purposes:
1) cleaving amplification products at the restriction enzyme site to produce precursors of hairpin-ended DNA molecules (see Section 5.3.1), which are further processed to produce hairpin-ended DNA molecules (see Section 5.3);
2) producing non-hairpin-ended DNA molecules for exonuclease digestion (see Section 5.4.1 and Section 5.4.2) after hairpin-ended DNA molecules have been generated, which assists the removal of undesired DNA molecules; and
3) cleaving amplification products at the unmethylated MSRE site while maintaining the DNA template intact because the MSRE site in the DNA template is methylated (see Section 5.2.4). Such process reduces viscosity of the amplification products and improves fidelity of the amplification (e.g., isothermal amplification, e.g., RCA, MDA).

In certain embodiments, the restriction enzyme site is located at a site outside of the inverted-repeat-flanked sequence of interest. In other words, the restriction enzyme site is located on the opposite side of both inverted repeats relative to the sequence of interest. In certain embodiments, the restriction enzyme site is not in the inverted repeat and/or is not in the sequence of interest and/or is not in any of the two regions between inverted repeat and sequence of interest. As a result, the hairpin-ended DNA molecule of interest does not comprise the restriction enzyme site.

In certain embodiments, the restriction enzyme site is unique, i.e., it is present only once in the DNA template. In certain embodiments, one DNA template comprises 1, 2, 3, 4, 5, 6, 7, 8, 9, 10 or more copies of the same restriction enzyme site. In certain embodiments, all copies of the same restriction enzyme site are located at a site outside of the inverted-repeat-flanked sequence of interest. In other words, all copies of the same restriction enzyme site are located on the opposite side of both inverted repeats relative to the sequence of interest. In certain embodiments, the restriction enzyme site is not in the inverted repeat and/or is not in the sequence of interest and/or is not in any of the two regions between inverted repeat and sequence of interest.

In certain embodiments, DNA templates disclosed herein and/or amplification products produced therefrom comprise at least two different restriction enzymes sites which are recognized by at least two different restriction enzymes.

In certain embodiments, the restriction enzyme sites are located outside of the inverted-repeat-flanked sequence of interest in a way that cleavage by the restriction enzyme(s) results in fragments of between 100 bp and 1,500 bp long. Generation of DNA fragments at this size range can be efficiently removed by size exclusion type of chromatography or digested by endonucleases, thus improving the process of removing undesired DNA molecules and increasing the purity of the hairpin-ended DNA molecules.

In certain embodiments, DNA templates disclosed herein and/or amplification products produced therefrom comprise an antibiotic resistant gene. In certain embodiments, at least one restriction enzyme site is located inside the antibiotic resistant gene and/or at least one restriction enzyme site is located inside the origin of replication. In certain embodiments, the resulting precursors of hairpin-ended DNA molecules and/or the non-hairpin-ended molecules after restriction enzyme digestion do not have a functional antibiotic resistance gene and/or an origin of replication.

In certain embodiments, the restriction site is symmetric with the two half-sites being adjacent. In certain embodiments, the restriction site is symmetric with the two half-sites are separated. In some embodiments, the restriction sites are asymmetric. In certain embodiments, the restriction enzyme cleaves within the recognition site. In certain embodiments, the restriction enzyme cleaves outside of the recognition site. In certain embodiments, the cleavage by the restriction enzyme results in blunt ends. In certain embodiments, the cleavage by the restriction enzyme results in a 5′ overhang. In certain embodiments, the cleavage by the restriction enzyme results in a 3′ overhang.

In certain embodiments, the recognition site for a restriction enzyme for the use with the methods and compositions provided herein is 6 or 8 nucleotides in length. In certain embodiments, the recognition site for a restriction enzyme is 12 to 45 nucleotides in length.

Illustrative restriction enzymes that can be used with the methods and compositions provided herein include Type II enzymes, Type IIS enzymes, Type IIb enzymes, Type Ile enzymes, and enzymes listed in Table 15. In certain embodiments, the restriction enzyme is an intron or an intein encoded (also known as homing endonuclease). In certain embodiments, the restriction enzyme is a fusion protein of two or more restriction enzymes. In certain embodiments, the restriction enzyme is an isoschizomer of Table 15. In certain embodiments, the restriction enzyme is a neoschizomer of Table 15. In certain embodiments, the precursor of hairpin-ended DNA molecule or the non-hairpin-ended molecules resulting from the cleavage by the restriction enzyme contain the restriction enzyme site. In certain embodiments, the precursor of hairpin-ended DNA molecule or the non-hairpin-ended molecules resulting from the cleavage by the restriction enzyme destroy the restriction enzyme site.

TABLE 15
Exemplary Restriction Enzymes and their Recognition Sites

| | | SEQ ID |
|---|---|---|
| Name | Recognition site | NO. |
| AatII | G↑ACGT↓C | 327 |
| AclI | AA↓CG↑TT | 328 |
| AcuI | CTGAAGNNNNNNNNNNNNNN↑NN↓ | 329 |
| AfeI | AGC↑↓GCT | 330 |
| Af1II | C↓TAA↑G | 331 |
| AgeI | A↓CCGG↑T | 332 |
| ApaLI | G↓TGCA↑C | 333 |
| AscI | GG↓CGCG↑CC | 334 |
| AseI | AT↓TA↑AT | 335 |
| AsiSI | GCG↑AT↓CGC | 336 |
| AvrII | C↓CTAG↑G | 337 |
| BamHI | G↓GATC↑C | 338 |
| BbsI | GAAGACNN↓NNNN↑ | 339 |
| BbvCI | CC↓TCA↑GC | 340 |
| BciVI | GTATCCNNNN↑N↓ | 341 |
| BfuAI | ACCTGCNNNN↓NNNN↑ | 342 |
| BgIII | A↓GATC↑T | 343 |
| BmgBI | CAC↑↓GTC | 344 |
| BmrI | ACTGGGNNNN↑N↓ | 345 |
| BmtI | G↑CTAG↓C | 346 |
| BpmI | CTGGAGNNNNNNNNNNNNNN↑NN↓ | 347 |
| BpuEI | CTTGAGNNNNNNNNNNNNNN↑NN↓ | 348 |
| BseRI | GAGGAGNNNNNNNN↑NN↓N | 349 |
| BseYI | C↓CCAG↑C | 350 |
| BsgI | GTGCAGNNNNNNNNNNNNNN↑NN↓ | 351 |
| BsiWI | C↓GTAC↑G | 352 |
| BsmBI | CGTCTCN↓NNNN↑ | 353 |
| BsmI | GAATG↑CN↓ | 354 |
| BspMI | ACCTGCNNNN↓NNNN↑ | 355 |
| BspQI | GCTCTTCN↓NNN↑ | 356 |
| BsrBI | CCG↑↓CTC | 357 |
| BsrDI | GCAATG↑NN↓ | 358 |
| BsrGI | T↓GTAC↑A | 359 |
| BssHII | G↓CGCG↑C | 360 |
| BssSI | C↓ACGA↑G | 361 |
| BstBI | TT↓CG↑AA | 362 |
| BstZ17I | GTA↑↓TAC | 363 |
| BtgZI | GCGATGNNNNNNNNNN↓NNNN↑ | 364 |
| BtsI | GCAGTG↑NN↓ | 365 |
| DraI | TTT↑↓AAA | 366 |
| EagI | C↓GGCC↑G | 367 |
| EarI | CTCTTCN↓NNN↑ | 368 |
| EciI | GGCGGANNNNNNNNN↑NN↓ | 369 |
| Eco53kI | GAG↑↓CTC | 370 |
| EcoP15I | CAGCAGNNNNNNNNNNNNNNNNNNNNNNNNN↓NN↑ | 371 |
| EcoRI | G↓AATT↑C | 372 |
| EcoRV | GAT↑↓ATC | 373 |
| HindIII | A↓AGCT↑T | 374 |
| HpaI | GTT↑↓AAC | 375 |
| I-CeuI | TAACTATAACGGTC↑CTAA↓GGTAGCGAA | 376 |
| I-SceI | TAGGG↑ATAA↓CAGGGTAAT | 377 |
| I-PpoI | CTCTC↑TTAA↓GGTAGC | 378 |
| KasI | G↓GCGC↑C | 379 |
| KpnI | G↓GTAC↑C | 380 |
| MfeI | C↓AATT↑G | 381 |
| MluI | A↓CGCG↑T | 382 |
| Nael | GCC↑↓GGC | 383 |
| NcoI | C↓CATG↑G | 384 |
| NdeI | CA↓TAN↑G | 385 |
| NgoMIV | G↓CCGG↑C | 386 |
| NheI | G↓CTAG↑C | 387 |
| NmeAIII | GCCGAGNNNNNNNNNNNNNNNNNNN↑N↓ | 388 |
| NotI | GC↓GGCC↑GC | 389 |
| NsiI | A↑TGCA↓T | 390 |
| PacI | TTA↑AT↓TAA | 391 |
| PaeR7I | C↓TCGA↑G | 392 |
| PaqCI | CACCTGCNNNN↓NNNN↑ | 393 |
| PciI | A↓CATG↑T | 394 |
| PluTI | G↑GCGC↓C | 395 |
| PmeI | GTTT↑↓AAAC | 396 |
| PmlI | CAC↑↓GTG | 397 |
| PsiI | TTA↑↓TAA | 398 |
| PstI | C↑TGCA↓G | 399 |
| PvuI | CG↑AT↓CG | 400 |
| PvuII | CAG↑↓CTG | 401 |
| SacI | G↑AGCT↓C | 402 |
| SacII | CC↑GC↓GG | 403 |
| SalI | G↓TCGA↑C | 404 |
| SapI | GCTCTTCN↓NNN↑ | 405 |
| SbfI | CC↑TGCA↓GG | 406 |
| ScaI | AGT↑↓ACT | 407 |
| SmaI | CCC↑↓GGG | 408 |
| SnaBI | TAC↑↓GTA | 409 |
| SpeI | A↓CTAG↑T | 410 |
| SphI | G↑CATG↓C | 411 |
| SrfI | GCCC↑↓GGGC | 412 |
| SspI | AAT↑↓ATT | 413 |
| SwaI | ATTT↑↓AAAT | 414 |
| TspMI | C↓CCGG↑G | 415 |
| XhoI | C↓TCGA↑G | 416 |
| XmaI | C↓CCGG↑G | 417 |
| ZraI | GAC↑↓GTC | 418 |
| Esp3I | CGTCTCN↓NNNN↑ | 419 |
| FseI | GG↑CCGG↓CC | 420 |
| FspI | TGC↑↓GCA | 421 |

Downward pointing arrows indicate the positions of cuts in the sequence shown. Upward pointing arrows indicate the positions of cuts in the complementary sequence. Double arrows (up/down) indicate a blunt cut site with the same position in both strands. N = A or C or G or T (i.e., any nt).

In certain embodiments, the restriction enzyme and/or its buffer are chosen to reduce or eliminate hydrolysis of DNA outside the specific target sequence for the restriction enzyme.

Instead of a restriction enzyme site, it is also possible to use two restriction sites recognized by nicking endonuclease (i.e., nicking endonuclease sites) on opposite strands such that digestion with the nicking endonuclease results in a double strand break similar to digestion with a restriction enzyme (see Section 5.1.4).

### (a) Methylation Sensitive Restriction Enzyme (MSRE) Sites

In certain embodiments, DNA templates disclosed herein and/or amplification products produced therefrom comprise a methylation sensitive restriction enzyme (MSRE) site. The MSRE site is methylated in the DNA template, and thus protects the DNA template from being cleaved by the MSRE. By contrast, the amplification product amplified from the DNA template comprises an unmethylated MSRE site, which is cleaved by the MSRE, and thus reduces the viscosity of the amplification product and improves the fidelity of the amplification (e.g., isothermal amplification, e.g., RCA, MDA).

Exemplary MSRE sites that can be used with presently disclosed subject matter are disclosed in Table 16. Exemplary MSRE that can be used for cleaving the MSRE sites are disclosed in Section 5.2.4.

TABLE 16
Exemplary MSRE and their Recognition Sites

| | | SEQ ID |
|---|---|---|
| MSRE | Recognition Site | NO. |
| SexAI | A↓CCWGG↑T | 422 |
| EcoRII | ↓CCWGG↑ | 423 |
| PspGI | ↓CCWGG↑ | 424 |
| GsuI | CTGGAGN₁₆↓ | 425 |
| SfoI | GGC↑↓GCC | 426 |
| NlaIV | GGN↑↓NCC | 427 |
| Asp718I | G↓GTAC↑C | 428 |
| Acc65I | G↓GTAC↑C | 429 |
| AvalI | G↓GWC↑C | 430 |
| BsaI | GGTCTCN₁↓N₄↑ | 431 |
| Eco47I | G↓GWC↑C | 432 |
| VpaK11BI | G↓GWC↑C | 433 |
| AlwNI | CAG↑NNN↓CTG | 434 |
| MscI | TGG↑↓CCA | 435 |
| NarI | GG↓CG↑CC | 436 |
| PflMI | CCAN↑NNN↓NTGG | 437 |
| PspOMI | G↓GGCC↑C | 438 |
| ApaI | G↑GGCC↓C | 439 |
| StuI | AGG↑↓CCT | 440 |
| BanI | G↓GYRC↑C | 441 |
| BsaHI | GR↓CG↑YC | 442 |
| BstXI | CCAN↑NNNN↓NTGG | 443 |
| EaeI | Y↓GGCC↑R | 444 |
| XbaI | T↓CTAG↑A | 445 |
| Eco0109I | RG↓GNC↑CY | 446 |
| PpuMI | RG↓GWC↑CY | 447 |
| Sau96I | G↓GNC↑C | 448 |
| ScrFI | CC↓N↑GG | 449 |
| StyD4I | ↓CCNGG↑ | 450 |
| BstKTI | G↑AT↓C | 451 |
| DpnII | ↓GATC↑ | 452 |
| MboI | ↓GATC↑ | 453 |
| MflI | R↓GATC↑Y | 454 |
| BspHI | T↓CATG↑A | 455 |
| AccIII | T↓CCGG↑A | 456 |
| BseAI | T↓CCGG↑A | 457 |
| BspEI | T↓CCGG↑A | 458 |
| AlwI | GGATC(4/5) | 459 |
| BclI | T↓GATC↑A | 460 |
| BsaBI | GATNN↑↓NNATC | 461 |
| BspDI | AT↓CG↑AT | 462 |
| ClaI | AT↓CG↑AT | 463 |
| HphI | GGTGAN₇↑N↓ | 464 |
| Hpy188I | TC↑N↓GA | 465 |
| Hpy188III | TC↓NN↑GA | 466 |
| MboII | GAAGAN₇↑N↓ | 467 |
| NruI | TCG↑↓CGA | 468 |
| TaqI | T↓CG↑A | 469 |

Downward pointing arrows indicate the positions of cuts in the sequence shown. Upward pointing arrows indicate the positions of cuts in the complementary sequence. Double arrows (up/down) indicate a blunt cut site with the same position in both strands. N = A or C or G or T (i.e., any nt); R = A or G (purine); W = A or T (weak); Y = C or T (pyrimidine). Runs of Ns outside the recognition sequence is shown as a single N with a subscript numeral.

In certain embodiments, the MSRE site is located outside of the inverted-repeat-flanked sequence of interest. In other words, the MSRE site is located on the opposite side of both inverted repeats relative to the sequence of interest. In certain embodiments, the MSRE site is not in the inverted repeat and/or is not in the sequence of interest and/or is not in any of the two regions between inverted repeat and sequence of interest. As a result, the hairpin-ended DNA molecule of interest does not comprise the MSRE site.

In certain embodiments, such an MSRE site is unique, i.e., it is present only once in the DNA template. In certain embodiments, one DNA template comprises 1, 2, 3, 4, 5, 6, 7, 8, 9, 10 or more copies of the same MSRE site. In certain embodiments, all copies of such MSRE site(s) are located outside of the inverted-repeat-flanked sequence of interest (in other words, they are located on the opposite side of both inverted repeats relative to the sequence of interest).

In certain embodiments, the MSRE site(s) as described in this section does/do not overlap with any of the primer binding sites described in Section 5.1.2.

In certain embodiments, the MSRE site in the DNA template is methylated. In certain embodiments, the DNA template is a double-stranded DNA molecule, and the MSRE sites in both strands are methylated. In certain embodiments, the DNA template is a double-stranded DNA molecule, and the MSRE site is methylated in one strand and unmethylated in the other strand (hemi-methylation). Any techniques known in the art can be used for methylating the MSRE site in the DNA template. In certain embodiments, the MSRE site is methylated by a DNA methyltransferase. In certain embodiments, the DNA methyltransferase is selected from the group consisting of dam methyltransferase, dem methyltransferase, CpG methyltransferase, SssI, EcoKI, and any combinations thereof. In certain embodiments, the methylation is a N4-methylcytosine, a 5-methylcytosine, a 5-hydroxymethylcytosine, a 6-methyladenine, a glucosylated-hydroxymethylcytosine, or a combination thereof. In certain embodiments, methods disclosed herein use a modification that could have the same effect as methylation, such as, 5-formylcytosine, queuosine, deoxyarchaeosine, or a 7-deazaguanine. Enzymes sensitive to other types of methylations (or modifications) can be found, for example, in rebase.neb.com.

In certain embodiments, the DNA template is methylated using an E. coli-based method. In certain embodiments, the method comprises co-transforming a plasmid, to be used as the double-stranded circular DNA, with a plasmid expressing a DNA methyltransferase to protect a restriction site from any restriction enzymes or MSREs. In certain embodiments, the method comprises using a strain engineered to express the DNA methyltransferase to protect a restriction site from any restriction enzymes or MSREs. In certain embodiments, the method comprises using a commercially available E. coli strain that expresses the DNA methyltransferase to protect a restriction site from any restriction enzymes or MSREs.

In certain embodiments, the DNA template is methylated using a cell-free system. In certain embodiments, the method comprises inserting a methylated DNA fragment into the DNA template. In certain embodiments, the methylated DNA fragment is synthesized artificially without the use of cells. In certain embodiments, the synthetic DNA fragment comprises additional nucleotide modifications (e.g., nucleotide modifications disclosed in paragraphs [0332]-[0337] of the present disclosure).

### 5.1.4 Additional Nicking Endonuclease Sites

In certain embodiments, DNA templates disclosed herein and/or amplification products produced therefrom comprise two additional nicking endonuclease sites (e.g., the fifth and the sixth nicking endonuclease sites) on the opposite strands such that digestion with the nicking endonuclease results in a double strand break similar to digestion with a restriction enzyme. In certain embodiments, a single-stranded DNA template comprises one additional nicking endonuclease site (e.g., the fifth or the sixth nicking endonuclease site). In certain embodiments, a double-stranded DNA template comprises two additional nicking endonuclease sites (e.g., the fifth and the sixth nicking endonuclease sites) on the opposite strands. The inclusion of additional nicking endonuclease sites in the amplification products serve similar purposes as the restriction enzyme site (see Section 5.1.3):
1) producing precursors of the hairpin-ended DNA molecules (see Section 5.3.1),
2) producing non-hairpin-ended DNA molecules for exonuclease digestion (see Section 5.4.1 and Section 5.4.2), and/or
3) reducing viscosity and improving fidelity of the amplification (e.g., isothermal amplification, e.g., RCA, MDA) with the inclusion of methylation-sensitive nicking endonuclease (MSNE) sites (see Section 5.2.4).

In certain embodiments, the pair of nicking endonuclease sites is located at a site outside of the inverted-repeat-flanked sequence of interest. In other words, the pair of nicking endonuclease sites is located on the opposite side of both inverted repeats relative to the sequence of interest. In certain embodiments, the pair of nicking endonuclease sites is not in the inverted repeat and/or is not in the sequence of interest and/or is not in any of the two regions between inverted repeat and sequence of interest. As a result, the hairpin-ended DNA molecule of interest does not comprise the pair of nicking endonuclease sites.

In certain embodiments, the pair of nicking endonuclease sites is unique, i.e., present only once in the DNA template. In certain embodiments, one DNA template comprises 1, 2, 3, 4, 5, 6, 7, 8, 9, 10 or more copies of the same pair of nicking endonuclease sites. In certain embodiments, all copies of the same pair of nicking endonuclease sites are located at a site outside of the inverted-repeat-flanked sequence of interest. In other words, all copies of the same embodiments, one DNA template comprises 1, 2, 3, 4, 5, 6, 7, 8, 9, 10 or more copies of the same pair of nicking endonuclease sites. In certain embodiments, all copies of the same pair of nicking nuclease sites are located on opposite sides of both inverted repeats relative to the sequence of interest. In certain embodiments, the pair of nicking nuclease sites are not in the inverted repeat and/or are not in the sequence of interest and/or is not in any of the two regions between the inverted repeat and sequence of interest.

In certain embodiments, the fifth and sixth nicking endonuclease sites comprised by the amplification products can be targeted and nicked by the same nicking endonuclease. In certain embodiments, the fifth and sixth nicking endonuclease sites comprised by the amplification products can be targeted and nicked by two different nicking endonucleases.

The fifth and sixth nicking endonuclease sites can be arranged in various configurations. In certain embodiments, the fifth and sixth nicking endonuclease sites are at least 10, at least 15, at least 20, at least 25, at least 30, at least 35, at least 40, at least 45, at least 50, at least 55, at least 60, at least 65, at least 70, at least 75, at least 80, at least 85, at least 86, at least 87, at least 88, at least 89, at least 90, at least 91, at least 92, at least 93, at least 94, at least 95, at least 96, at least 97, at least 98, at least 99, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, or at least 200 nucleotides apart. In certain embodiments, the fifth and sixth nicking endonuclease sites are about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 105, about 110, about 115, about 120, about 125, about 130, about 135, about 140, about 145, about 150, about 155, about 160, about 165, about 170, about 175, about 180, about 185, about 190, about 195, or about 200 nucleotides apart.

In certain embodiments, each nicking endonuclease site for the use with the presently disclosed subject matter is 6, 7, or 8 nucleotides in length. In certain embodiments, the nicking endonuclease site is between about 5 and about 20, between about 5 and about 15, between about 5 and about 10, between about 10 and about 20, between about 10 and about 15, or between about 15 and about 20 nucleotides long. In certain embodiments, the nicking endonuclease site is about 5, about 10, about 15, about 20, or more nucleotides in length.

Illustrative nicking endonucleases that can be used with the methods and compositions provided herein are disclosed in Section 5.3.2 (e.g., Table 21). In certain embodiments, the nicking endonuclease and/or its buffer are chosen to reduce or eliminate hydrolysis of DNA outside the specific target sequence for the nicking endonuclease.

### (a) Methylation Sensitive Nicking Endonuclease (MSNE) Sites

In certain embodiments, DNA templates disclosed herein and/or amplification products produced therefrom comprise two methylation sensitive nicking endonuclease (MSNE) sites on the opposite strands such that digestion with the nicking endonuclease results in a double strand break similar to digestion with a restriction enzyme. In certain embodiments, a single-stranded DNA template comprises one MSNE. In certain embodiments, a double-stranded DNA template comprises two MSNE sites on the opposite strands. The MSNE sites are methylated in the DNA template, and thus protect the DNA template from being cleaved by the MSNE. By contrast, the amplification product amplified from the DNA template comprises unmethylated MSNE sites, which are recognized and nicked by the MSNE, and thus reduces the viscosity of the amplification product and improves the fidelity of the amplification (e.g., isothermal amplification, e.g., RCA, MDA).

Exemplary MSNE sites that can be used with presently disclosed subject matter is provided in Table 17. Exemplary MSNE that can be used for nicking at the MSNE sites are disclosed in Section 5.2.4.

TABLE 17
Exemplary MSNEs and their Recognition Sites

| MSNE | Recognition Sites | SEQ ID NO |
|---|---|---|
| Nt.AlwI | GGATCNNNN↓ | 470 |
| Nt.BsaI | GGTCTCN↓ | 471 |

Where, in Table 17, downward pointing arrows indicate the positions of cuts in the sequence shown and the absence of an upward pointing arrow indicates that the complementary sequence is not cut.

In certain embodiments, the pair of MSNE sites is located outside of the inverted-repeat-flanked sequence of interest. In other words, the pair of MSNE sites is located on the opposite side of both inverted repeats relative to the sequence of interest. In certain embodiments, the pair of MSNE sites is not in the inverted repeat and/or is not in the sequence of interest and/or is not in any of the two regions between the inverted repeat and sequence of interest. As a result, the hairpin-ended DNA molecule of interest (i.e., the hairpin-ended DNA molecule comprising the sequence of interest) does not comprise the MSNE sites.

In certain embodiments, one DNA template comprises 1, 2, 3, 4, 5, 6, 7, 8, 9, 10 or more copies of the same MSNE site. In certain embodiments, all copies of such MSNE site(s) are located outside of the inverted-repeat-flanked sequence of interest (in other words, they are located on the opposite side of both inverted repeats relative to the sequence of interest).

In certain embodiments, the MSNE site(s) as described in this section does/do not overlap with any of the primer binding sites described in Section 5.1.2.

In certain embodiments, the MSNE site in the DNA template is methylated. In certain embodiments, the DNA template is a double-stranded DNA molecule, and the MSNE sites in both strands are methylated. In certain embodiments, the DNA template is a double-stranded DNA molecule, and the MSNE site is methylated in one strand and unmethylated in the other strand (hemi-methylation). Any techniques known in the art can be used for methylating the MSNE site in the DNA template. In certain embodiments, the MSNE site is methylated by a DNA methyltransferase. In certain embodiments, the DNA methyltransferase is selected from the group consisting of dam methyltransferase, dem methyltransferase, CpG methyltransferase, SssI, EcoKI, and any combinations thereof. In certain embodiments, the methylation is a 4-methylcytosine, a 5-methylcytosine, a 5-hydroxymethylcytosine, a 6-methyladenine, a glucosylated-hydroxymethylcytosine, or a combination thereof. In certain embodiments, methods disclosed herein use a modification that could have the same effect as methylation such as 5-formylcytosine, queuosine, deoxyarchaeosine, or 7-deazaguanine. Enzymes sensitive to other types of methylations (or modifications) can be found, in rebase.neb.com.

In certain embodiments, the DNA template is methylated using an E. coli-based method. In certain embodiments, the method comprises co-transforming a plasmid, to be used as the double-stranded circular DNA, with a plasmid expressing a DNA methyltransferase. In certain embodiments, the method comprises using a strain engineered to express the DNA methyltransferase. In certain embodiments, the method comprises using a commercially available E. coli strain that express the DNA methyltransferase.

In certain embodiments, the DNA template is methylated using a cell-free system. In certain embodiments, the method comprises inserting a methylated DNA fragment into the DNA template. In certain embodiments, the methylated DNA fragment is synthesized artificially without the use of cells. In certain embodiments, the synthetic DNA fragment comprises additional nucleotide modifications (e.g., nucleotide modifications disclosed in paragraphs [0332]-[0337] of the present disclosure).

### 5.1.5 Viral DNA Sequence Features Absent in the DNA Molecules Provided Herein

Certain suitable and desired sequence features or elements can be included in the DNA molecules provided herein (e.g., DNA templates, amplification products, and hairpin-ended DNA molecules) or excluded from the DNA molecules provided herein.

DNA sequence elements or features that can be excluded from the DNA molecules provided herein can be a viral replication-associated protein binding sequence (“RABS”), which refers to a DNA sequence to which a viral DNA replication-associated protein (“RAP”) or an isoform thereof, encoded by the Parvoviridae gene Rep or NS1 can bind. In certain embodiments, the RABS is a Rep binding sequence (“RBS”). Rep can bind to two elements within the ITR. It can bind to a nucleotide sequence in the stem structure of the ITR (i.e., the nucleotide sequence recognized by a Rep protein for replication of viral nucleic acid molecules). Such a RBS is also referred to as RBE (Rep-binding element). Rep can also bind to a nucleotide sequence which forms a small palindrome comprising a single tip of an internal hairpin within the ITR, thereby stabilizing the association between Rep and the ITR. Such a RBS is also referred to as RBE′. In certain embodiments, the RABS is an NS1-binding element (“NSBE”) to which replication-associated viral protein NS1 can bind. In certain embodiments, Rep can bind to a nucleotide sequence in the stem structure of the ITR (i.e., the nucleotide sequence recognized by a Rep or NS1 protein (for replication of viral nucleic acid molecules)) and/or the site of specific interaction between the Rep and/or NS1 protein and the nucleotide sequence. A RABS can be a sequence of 5 nucleotides to 300 nucleotides. In certain embodiments of the DNA molecules provided herein (e.g., DNA molecules of Sections 3 and 5.1), the RABS can be a sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, or at least 400 nucleotides. In certain embodiments, the RABS can be a sequence of about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, or about 400 nucleotides.

Alternatively, the DNA molecules provided herein (e.g., DNA molecules of Sections 3 and 5.1) can lack a functional RABS by functionally inactivating the RABS sequence present in the DNA molecules with mutations, insertions, and/or deletions (including partial deletions or truncations), such that the RABS can no longer serve as a recognition and/or binding site for the Rep protein or NS1 protein. As such, in certain embodiments of the DNA molecules provided herein (e.g., DNA molecules of Sections 3 and 5.1), the DNA molecule can comprise a functionally inactivated RABS. Such functional inactivation can be assessed by measuring and comparing the binding between the Rep or NS1 protein and the DNA molecules comprising the functionally inactivated RABS with that between the Rep or NS1 proteins and a reference molecule comprising the wild type (wt) RBS or NSBE sequences (e.g., the same DNA molecule but with wt RBS or wt NSBE sequences). Such binding can be determined by any binding measurements known and used in the field of molecular biology, for example, chromatin immunoprecipitation (ChIP) assays, DNA electrophoretic mobility shift assay (EMSA), DNA pull-down assays, or microplate capture and detection assays, as further described in Matthew J. Guille & G. Geoff Kneale, Molecular Biotechnology 8:35-52 (1997); Bipasha Dey et al., Mol Cell Biochem. 2012 June; 365 (1-2): 279-99, both of which are hereby incorporated in their entireties by reference. In certain embodiments, the binding between the RAPs and the functionally inactivated RABS in the DNA molecule is at most 0.001%, at most 0.01%, at most 0.1%, at most 1%, at most 1.5%, at most 2%, at most 2.5%, at most 3%, at most 3.5%, at most 4%, at most 4.5%, at most 5%, at most 5.5%, at most 6%, at most 6.5%, at most 7%, at most 7.5%, at most 8%, at most 8.5%, at most 9%, at most 9.5%, or at most 10%, compared to the binding between the RAPs and the wild type RBS or NSBE in a reference DNA molecule (e.g., the same DNA molecule but with a wild type RBS or NSBE sequence). In certain embodiments, the binding between the RAPs and the functionally inactivated RABS in the DNA molecule is about 0.001%, about 0.01%, about 0.1%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10%, compared to the binding between the RAPs and the wild type RABS in a reference DNA molecule (e.g., the same DNA molecule but with a wt RBS or NSBE sequence). In certain embodiments, the binding between the RAPs and the functionally inactivated RABS in the DNA molecule is 0.001%, 0.01%, 0.1%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%, compared to the binding between the RAPs and the wild type RABS in a reference DNA molecule (e.g., the same DNA molecule but with a wt RBS or NSBE sequence).

Furthermore, the DNA molecules provided herein (e.g., DNA molecules of Sections 3 and 5.1) can lack a functional RAPs or viral capsid encoding sequence by functionally inactivating the Rep protein, NS1 or viral capsid encoding sequence present in the DNA molecules with mutations, insertions, and/or deletions (including partial deletions or truncations), such that the RAPs or viral capsid encoding sequence can no longer functionally express the Rep protein, NS1 protein, or viral capsid protein. Such functional inactivating mutations, insertions, or deletions can be achieved, for example: by using mutations, insertions, and/or deletions to shift the open reading frame of Rep protein, NS1 protein, or viral capsid encoding sequence; by using mutations, insertions, and/or deletions to remove the start codon; by using mutations, insertions, and/or deletions to remove the promoter or transcription initiation site; by using mutations, insertions, and/or deletions to remove the RNA polymerase binding sites; by using mutations, insertions, and/or deletions to remove the ribosome recognition or binding sites; or by other means known and used in the field.

In certain embodiments, the DNA molecules provided herein (e.g., DNA molecules of Sections 3 and 5.1) can comprise an RBS inactivated by mutation. In certain embodiments, the DNA molecules can comprise an RBS inactivated by a mutation of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in the RBS. In certain embodiments, the DNA molecule comprises an RBS inactivated by a mutation of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% of the nucleotides in the RBS. In certain embodiments, the DNA molecule comprises an RBS inactivated by a deletion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in the RBS. In certain embodiments, the DNA molecule comprises an RBS inactivated by a deletion of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% of the nucleotides in the RBS. In certain embodiments, the deletion of the preceding sentence is an internal deletion, a deletion from the 5′ end, or a deletion from the 3′ end. In certain embodiments, the deletion of this paragraph can be any combination of internal deletions, deletions from the 5′ end, and/or deletions from the 3′ end. In certain embodiments, the DNA molecule comprises an RBS inactivated by a deletion of the entire RBS sequences. In some additional embodiments, the DNA molecule comprises an RBS inactivated by a partial deletion of the RBS sequences.

In certain embodiments, the DNA molecule comprises an NBSE inactivated by mutation. In certain embodiments, the DNA molecule comprises an NSBE inactivated by a mutation of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in the NSBE. In certain embodiments, the DNA molecule comprises an NSBE inactivated by a mutation of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% of the nucleotides in the NSBE. In certain embodiments, the DNA molecule comprises an NSBE inactivated by a deletion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in the NSBE. In certain embodiments, the DNA molecule comprises an NSBE inactivated by a deletion of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% of the nucleotides in the NSBE. In certain embodiments, the deletion of the preceding sentence is an internal deletion, a deletion from the 5′ end, or a deletion from the 3′ end. In certain embodiments, the deletion of this paragraph can be any combination of internal deletions, deletions from the 5′ end, and/or deletions from the 3′ end. In certain embodiments, the DNA molecule comprises an NSBE inactivated by a deletion of the entire NSBE sequences. In some additional embodiments, the DNA molecule comprises an NSBE inactivated by a partial deletion of the NSBE sequences.

Similarly, DNA sequence elements or features can be included or excluded from any specific regions of the DNA molecules provided herein (see Section 5.1) or any specific regions of the DNA molecules used in the methods provided herein (see Sections 5.2-5.4). In certain embodiments, the DNA molecule lacks a Rep protein encoding sequence. In certain embodiments, the DNA molecule lacks a NS1 protein encoding sequence. In certain embodiments, the DNA molecule lacks a viral capsid protein encoding sequence. In certain embodiments, the expression cassette lacks a Rep protein encoding sequence. In certain embodiments, the expression cassette lacks a NS1 protein encoding sequence. In certain embodiments, the expression cassette lacks a viral capsid protein encoding sequence. In certain embodiments, the DNA molecule lacks an RABS. In certain embodiments, the first inverted repeat lacks an RABS. In certain embodiments, the second inverted repeat lacks an RABS. In certain embodiments, the DNA sequence between the ITR closing base pair of the first inverted repeat and the ITR closing base pair of the second inverted repeat lacks an RABS. The lack of an RABS can be the lack of one RABS, the lack of two RABSs, the lack of more than two RABs, or the lack of any RABS. In certain embodiments, the DNA molecule comprises a functionally inactivated Rep protein encoding sequence. In certain embodiments, the DNA molecule comprises a functionally inactivated Rep protein encoding sequence. In certain embodiments, the DNA molecule comprises a functionally inactivated NS1 protein recognition sequence. In certain embodiments, the DNA molecule comprises a functionally inactivated NS1 protein encoding sequence. In certain embodiments, the DNA molecule comprises a functionally inactivated viral capsid protein encoding sequence. In certain embodiments, the expression cassette comprises a functionally inactivated Rep protein encoding sequence. In certain embodiments, the expression cassette comprises a functionally inactivated NS1 protein encoding sequence. In certain embodiments, the expression cassette comprises a functionally inactivated viral capsid protein encoding sequence. In certain embodiments, the DNA molecule comprises a functionally inactivated RABS. In certain embodiments, the first inverted repeat comprises a functionally inactivated RABS. In certain embodiments, the second inverted repeat comprises a functionally inactivated RABS. In certain embodiments, the DNA sequence between the ITR closing base pair of the first inverted repeat and the ITR closing base pair of the second inverted repeat comprises a functionally inactivated RABS. It is contemplated that one, two, or more RABS or all RABSs can be functionally inactivated.

Additionally, DNA sequence elements or features can be functionally inactivated from any specific regions of the DNA molecules provided herein (see Section 5.1) or any specific regions of the DNA molecules used in the methods provided herein (see Sections 5.2-5.4). In certain embodiments, the first inverted repeat comprises a functionally inactivated RABS and the second inverted repeat comprises a functionally inactivated RABS. In certain embodiments, the first inverted repeat comprises a functionally inactivated RABS and the DNA sequence between the ITR closing base pair of the first inverted repeat and the ITR closing base pair of the second inverted repeat comprises a functionally inactivated RABS. In certain embodiments, the second inverted repeat comprises a functionally inactivated RABS and the DNA sequence between the ITR closing base pair of the first inverted repeat and the ITR closing base pair of the second inverted repeat comprises a functionally inactivated RABS. In certain embodiments, the first inverted repeat comprises a functionally inactivated RABS, the second inverted repeat comprises a functionally inactivated RABS, and the DNA sequence between the ITR closing base pair of the first inverted repeat and the ITR closing base pair of the second inverted repeat comprises a functionally inactivated RABS. It is contemplated that one, two, or more RABS or all RABSs can be functionally inactivated.

As described in Sections 3, 5.1.1(a), and 5.1.1(e), such DNA sequence elements or features that can be excluded from the DNA molecules provided herein can be a terminal resolution site (“TRS”). A TRS refers to a nucleotide sequence in the inverted repeat of the DNA molecules that is recognized by a RAP (for replication of viral nucleic acid molecules) and is the site of strand-specific cleavage by the endonuclease activity of the RAP protein. The TRS is also the site of specific interaction between the RAP and the nucleotide sequence. Nucleotide sequences of the conserved sites of specific cleavage by the endonuclease activity of the RAP proteins can be determined by any DNA nicking assay known and used in the field of molecular biology, for example, gel electrophoresis, fluorophore-based in vitro nicking assays, radioactive in vitro nicking assay, as further described in Xu P, et al 2019. Antimicrob Agents Chemother 63: e01879-18.; US20190203229A; both of which are hereby incorporated in their entireties by reference. In certain embodiments, a TRS can be a nucleotide sequence in the inverted repeat of the DNA molecules that is recognized by a Rep protein (for replication of viral nucleic acid molecules) and is the site of strand specific nicking by the endonuclease activity of the Rep protein. The TRS can also be the site of specific cleavage by the endonuclease activity of the Rep protein. In certain embodiments, a TRS can be a nucleotide sequence in the inverted repeat of the DNA molecules that is recognized by a NS1 protein (for replication of viral nucleic acid molecules) and is the site of strand specific nicking by the endonuclease activity of the NS1 protein. In certain embodiments, the TRS can also include the site of specific interaction between the NS1 protein and the nucleotide sequence. TRS can be a sequence of 5 nucleotides to 300 nucleotides. In certain embodiments, the TRS can be a sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, or at least 400 nucleotides. In certain embodiments, the TRS can be a sequence of about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, or about 400 nucleotides.

Alternatively, the DNA molecules provided herein can lack a functional TRS by functionally inactivating the TRS sequence present in the DNA molecules with mutations, insertions, and/or deletions (including partial deletions or truncations), such that the TRS can no longer serve as a recognition and/or binding site for the RAP (i.e., Rep and NS1). As such, in certain embodiments, the DNA molecules provided herein comprise a functionally inactivated TRS. Such functional inactivation can be assessed by measuring and comparing the binding between the RAP (i.e., Rep and NS1) and the DNA molecules comprising the functionally inactivated TRS with that between the RAP and a reference molecule comprising the wild type (wt) TRS sequences (e.g., the same DNA molecule but with a wt TRS sequence). Such binding can be determined by any binding measurements known and used in the field of molecular biology, for example, chromatin immunoprecipitation (ChIP) assays, DNA electrophoretic mobility shift assay (EMSA), DNA pull-down assays, or microplate capture and detection assays, as further described in Matthew J. Guille & G. Geoff Kneale, Molecular Biotechnology 8:35-52 (1997); Bipasha Dey et al., Mol Cell Biochem. 2012 June; 365(1-2):279-99, both of which are hereby incorporated in their entireties by reference. In certain embodiments, the binding between the RAP (i.e., Rep and NS1) and the functionally inactivated TRS in the DNA molecule is at most 0.001%, at most 0.01%, at most 0.1%, at most 1%, at most 1.5%, at most 2%, at most 2.5%, at most 3%, at most 3.5%, at most 4%, at most 4.5%, at most 5%, at most 5.5%, at most 6%, at most 6.5%, at most 7%, at most 7.5%, at most 8%, at most 8.5%, at most 9%, at most 9.5%, or at most 10%, compared to the binding between the RAP (i.e., Rep and NS1) and the wild type TRS in a reference DNA molecule (e.g., the same DNA molecule but with a wt TRS sequence). In certain embodiments, the binding between the RAP (i.e., Rep and NS1) and the functionally inactivated TRS in the DNA molecule is about 0.001%, about 0.01%, about 0.1%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10%, compared to the binding between the RAP (i.e., Rep and NS1) and the wild type TRS in a reference DNA molecule (e.g., the same DNA molecule but with a wt TRS sequence). In certain embodiments, the binding between the RAP (i.e., Rep and NS1) and the functionally inactivated TRS in the DNA molecule is 0.001%, 0.01%, 0.1%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%, compared to the binding between the RAP (i.e., Rep and NS1) and the wild type TRS in a reference DNA molecule (e.g., the same DNA molecule but with a wt TRS sequence).

In certain embodiments, the DNA molecule comprises a TRS inactivated by mutation. In certain embodiments, the DNA molecule comprises a TRS inactivated by a mutation of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in the TRS. In certain embodiments, the DNA molecule comprises a TRS inactivated by a mutation of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% of the nucleotides in the TRS. In certain embodiments, the DNA molecule comprises a TRS inactivated by a deletion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in the TRS. In certain embodiments, the DNA molecule comprises a TRS inactivated by a deletion of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% of the nucleotides in the TRS. In certain embodiments, the deletion of the preceding sentence is an internal deletion, a deletion from the 5′ end, or a deletion from the 3′ end. In certain embodiments, the deletion of this paragraph can be any combination of internal deletions, deletions from the 5′ end, and/or deletions from the 3′ end. In certain embodiments, the DNA molecule comprises a TRS inactivated by a deletion of the entire TRS sequences. In some additional embodiments, the DNA molecule comprises a TRS inactivated by a partial deletion of the TRS sequences.

Similarly, DNA sequence elements or features can be included or excluded from any specific regions of the DNA molecules provided herein (see Section 5.1) or any specific regions of the DNA molecules used in the methods provided herein (see Sections 5.2-5.4). In certain embodiments, the DNA molecule lacks a TRS. In certain embodiments, the first inverted repeat lacks a TRS. In certain embodiments, the second inverted repeat lacks a TRS. In certain embodiments, the first inverted repeat lacks a TRS and the second inverted repeat lacks a TRS.

Alternatively, TRS sequence elements or features can be functionally inactivated from any specific regions of the DNA molecules provided herein (see Section 5.1) or any specific regions of the DNA molecules used in the methods provided herein (see Sections 5.2-5.4). In certain embodiments, the DNA molecule comprises a functionally inactivated TRS. In certain embodiments, the first inverted repeat comprises a functionally inactivated TRS. In certain embodiments, the second inverted repeat comprises a functionally inactivated TRS. In certain embodiments, the first inverted repeat comprises a functionally inactivated TRS and the second inverted repeat comprises a functionally inactivated TRS.

In certain embodiments, the RABS excluded or functionally inactivated in the DNA molecules provided herein can be any, or any combination of any number, or all of the RABS sequences listed in Table 18 and their reverse complementary sequences. Further non-limiting examples of terminal repeats for DNA molecules lacking RBS sequences provided in SEQ ID NOs: 1, 2, 3, 5, 6, and 7.

TABLE 18
Exemplary RAPs

| | SEQ ID | Corresponding RABS | SEQ ID | Reverse Complement |
|---|---|---|---|---|
| RAPS | NO | Sequences | NO | RABS |
| Rep | 472 | GCTCGCTCGCTC | 473 | GAGCGAGCGAGC |
| (AAV1, 2, 7) | | | | |
| Rep (AAV3) | 474 | GATCGCTCGCTC | 475 | GAGCGAGCGATC |
| Rep (AAV4) | 476 | GCTCGCTCACTCACTC | 477 | GAGTGAGTGAGCGAGC |
| Rep (AAV5) | 478 | GCTCGCTCGCTGGCTC | 479 | GAGCCAGCGAGCGAGC |
| NS1-NSBEI | 480 | GCCGCCGG | 481 | CCGGCGGC |
| (B19V) | | | | |
| NS1-NSBE2 | 482 | GGCGGGAC | 483 | GTCCCGCC |
| (B19V) | | | | |
| NS1-NSBE3 | 484 | TTCCGGTACA | 485 | TGTACCGGAA |
| (B19V) | | | | |

In certain embodiments, the DNA molecules can lack encoding sequences for any one, or any combination of any number, or all of the RAPs described in Table 18. In certain embodiments, the DNA molecules comprise functionally inactivated sequences encoding for any one, or any combination of any number, or all of the RAPs described in Table 18. In certain embodiments, the DNA molecules comprise functionally inactivated sequences encoding for any one, or any combination of any number, or all of the RAPs described in Table 18.

In certain embodiments, the DNA molecule comprises functionally inactivated recognition sequences for any one, or any combination of any number, or all of the RAPs described in Table 18. In certain embodiments, one or both hairpinned inverted repeats lack the RAPS recognition sequence:

(SEQ ID NO: 486)
GGCCACTCCCGAAGAGCGCGCTCGCTATCTCACTGAGGCCGGGCG
ACCAAAGGTCGCCCGACGCCCGGGCTTTGCCCGGGCGGCCTCAGT
GAGATAGCGAGCGCGCTCTTCGGGAGTGGCC

In certain embodiments, the TRS excluded or functionally inactivated in the DNA molecules provided herein can be any, or any combination of any number, or all of the TRS sequences listed in the following Table 19.

TABLE 19
Exemplary RAPs

| | Corresponding | SEQ ID |
|---|---|---|
| RAP (Virus) | TRS sequences | NO |
| Rep (AAV2, | AGTTGG | 487 |
| AAV3, AAV4) | | |
| Rep (AAV1, | AGTTGC | 488 |
| AAV6) | | |
| Rep(AAV5) | AGTGTGGC | 489 |
| NS1 (B19) | GACACC | 490 |
| NS1 (HBOV) | CTATATCT | 491 |
| NS1 (MVM) | CTWWTCA (W = A/T) | 492 |

As the methods provided herein do not need a viral replication step and the DNA molecules provided herein do not need to be produced or replicated in a virus life cycle, the disclosure provides and a person reading the disclosure would understand that the DNA molecules provided herein can lack various DNA sequences or features, including those sequences or features provided in Section 5.1.5. DNA molecules lacking RABS and/or TRS and DNA molecules comprising functionally inactivated RABS and/or functionally inactivated TRS as provided in Section 5.1.5 provide at least a major advantage in that the DNA molecules would have no or significantly lower risk of mobilization or replication once administered to a patient when compared with DNA molecules including such RABS and/or TRS sequences. Risk of mobilization or mobilization risk refers to the risk of the replication-defective DNA molecules reverting to replication or production of viral particles in the host that has been administered the DNA molecules. Such mobilization risk can result from the presence of viral proteins (e.g., Rep proteins, NS1 proteins or viral capsid proteins) expressed by viruses that have infected the same host that has been administered the DNA molecules. Mobilization risk poses a significant safety concern for using the replication defective viral genome as gene therapy vectors, as described for example in Liujiang Song, Hum Gene Ther, 2020 October; 31(19-20):1054-1067 (incorporated herein in its entirety by reference). Such DNA molecules lacking RABS and/or TRS would have no binding site for viral RAP to initiate the replication even if other helper viruses are present in the same host to provide RAPs. Without being bound by theory, it is thought that replication initiation may require helper factors, which are provided by coinfections of the host by auxiliary viruses, referred to “helper viruses” which can include viruses from the herpesvirus family, adenoviruses, and papillomaviruses.

Accordingly, in certain embodiments of the DNA molecules provided herein including those in Section 5.1.5, the DNA molecules without RABS and/or without TRS have less mobilization risk after administration to a subject or a patient when compared with DNA molecules with RABS and/or with TRS. In certain embodiments of the DNA molecules provided herein including those in Section 5.1.5, the DNA molecules comprising functionally inactivated RABS and/or functionally inactivated TRS have less mobilization risk after administration to a subject or a patient when compared with DNA molecules with RABS and/or with TRS. Such reduction of mobilization risk can be determined as (Pm−Po)/Pm, wherein Pm is the number of viral particles produced from the control DNA molecules with RABS when RAPs are present (e.g., due to the infection of any virus comprising RAPs or engineered expression of RAPs in the same host) and Po is the number of viral particles produced from DNA molecules lacking RABS or comprising functionally inactivated RABS as provided herein under comparable conditions in the same host used for the control DNA molecules. Alternatively, such reduction of mobilization risk can be determined as (Pm−Po)/Pm, wherein Pm is the number of viral particles produced from the control DNA molecules with TRS when RAPs are present (e.g., due to the infection of any virus comprising RAPs or engineered expression of RAPs in the same host) and Po is the number of viral particles produced from DNA molecules lacking TRS or comprising functionally inactivated TRS as provided herein under comparable conditions in the same host used for the control DNA molecules. Additionally, such reduction of mobilization risk can be determined as (Pm−Po)/Pm, wherein Pm is the number of viral particles produced from the control DNA molecules with RABS and with TRS when RAPs are present (e.g., due to the infection of any virus comprising Rep proteins or NS1 proteins or engineered expression of Rep proteins or NS1 proteins in the same host) and Po is the number of viral particles produced from DNA molecules (i) lacking RABS or comprising functionally inactivated RABS and (ii) lacking TRS or comprising functionally inactivated TRS as provided herein under comparable conditions in the same host used for the control DNA molecules. As described in Liujiang Song, Hum Gene Ther, 2020 October; 31(19-20):1054-1067 (incorporated herein in its entirety by reference), the host used for determining the particle numbers produced can be cells, animals (e.g., mouse, hamster, rate, dog, rabbit, guinea pig, and other suitable mammals), or human. The disclosure further provides and a person of ordinary skill in the art reading the disclosure would understand that Pm and Po, each as described in this paragraph, can be used also to determine the absolute or relative levels of mobilization. Briefly, in such an assay, the DNA molecules are transfected into the host cells (e.g., HEK293 cells) or transduced into the host cells by infecting with a viral particle comprising DNA molecules. The host cells are further transfected with Rep protein, NS1 protein, or co-infected with another virus expressing the Rep protein or NS1 protein (for example wild type viruses). The host cells are then cultured to produce and release viral particles. Virions are then harvested by collecting both the host cell and the culture media after culturing 48 to 72 hours (e.g., 65 hours). The titer for the viral particles (proxy for Pm and Po) can be determined by a probe-based quantitative PCR (qPCR) analysis following benzonase treatment to eliminate nonencapsidated DNA, as described in Song et al., Cytotherapy 2013; 15:986-998, which is incorporated in its entirety by reference. An exemplary implementation of such assay is provided in Liujiang Song, Hum Gene Ther, 2020 October; 31(19-20):1054-1067, which is incorporated herein in its entirety by reference.

Based on the determination of the reduction of mobilization risk and the mobilization risk levels, in certain embodiments of the DNA molecules provided herein including in Section 5.1.5, the mobilization risk of the DNA molecules when administered to a host is lower than control DNA molecules with RABS and/or with TRS by 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, or 20%. In certain embodiments, the mobilization risk of the DNA molecules when administered to a host is lower than control DNA molecules with RABS and/or with TRS by at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 79%, at least 78%, at least 77%, at least 76%, at least 75%, at least 74%, at least 73%, at least 72%, at least 71%, at least 70%, at least 69%, at least 68%, at least 67%, at least 66%, at least 65%, at least 64%, at least 63%, at least 62%, at least 61%, at least 60%, at least 59%, at least 58%, at least 57%, at least 56%, at least 55%, at least 54%, at least 53%, at least 52%, at least 51%, at least 50%, at least 49%, at least 48%, at least 47%, at least 46%, at least 45%, at least 44%, at least 43%, at least 42%, at least 41%, at least 40%, at least 39%, at least 38%, at least 37%, at least 36%, at least 35%, at least 34%, at least 33%, at least 32%, at least 31%, at least 30%, at least 29%, at least 28%, at least 27%, at least 26%, at least 25%, at least 24%, at least 23%, at least 22%, at least 21%, or at least 20%. In certain embodiments, the mobilization risk of the DNA molecules when administered to a host is lower than control DNA molecules with RABS and/or with TRS by about 100%, about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, or about 20%.

Alternatively, in certain embodiments, the DNA molecules provided herein including in Section 5.1.5, result in no detectable mobilization (e.g., based on the measurement of Po provided in Section 5.1.5). In certain embodiments, the DNA molecules provided herein in Section 5.1.5 result in mobilization of no more than 0.0001%, no more than 0.001%, no more than 0.01%, no more than 0.1%, no more than 1%, no more than 1.5%, no more than 2%, no more than 2.5%, no more than 3%, no more than 3.5%, no more than 4%, no more than 4.5%, no more than 5%, no more than 5.5%, no more than 6%, no more than 6.5%, no more than 7%, no more than 7.5%, no more than 8%, no more than 8.5%, no more than 9%, no more than 9.5%, or no more than 10% of the mobilization resulted from a reference DNA molecule (e.g., the same DNA molecule but with a wild type RABS and/or with wild type TRS sequence). In certain embodiments, the DNA molecules provided herein result in mobilization of about 0.0001%, about 0.001%, about 0.01%, about 0.1%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% of the mobilization resulted from a reference DNA molecule (e.g., the same DNA molecule but with a wild type RABS and/or with wild type TRS sequence). In a certain embodiments, the DNA molecules provided herein result in mobilization of 0.0001%, 0.001%, 0.01%, 0.1%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the mobilization resulted from a reference DNA molecule (e.g., the same DNA molecule but with a wild type RABS and/or with wild type TRS sequence). Such percentage of mobilization can be determined by using the Pm and Po determined as further described in the preceding paragraphs (including the preceding 2 paragraphs).

In certain embodiments, the DNA molecules provided herein (for example, as in Section 5.1.5) comprise ITRs that have functionally inactivated RABS and/or functionally inactivated TRS. In certain embodiments, the DNA molecules provided herein comprise ITRs that lack an RABS and/or a TRS. In certain embodiments, the DNA molecules provided herein have less mobilization risk after being administered to a subject or a patient when compared with DNA molecules comprising a functional RABS and/or functional TRS. ITRs that have functionally inactivated RABS and/or functionally inactivated TRS are referred to as “viral replication deficient inverted repeats” or “viral replication deficient inverted terminal repeats”, interchangeably.

In certain embodiments, the methods provided herein do not require any RABS. In certain embodiments, the DNA molecules provided herein do not need to be produced and/or replicated in a virus life cycle. The person of skill in the art would understand that the DNA molecules provided herein can lack additional features traditionally associated with RABS and/or viral production or replication, including those sequences or features discussed, for example, in Section 5.1.5. In certain embodiments, the DNA molecules lacking RBS and/or DNA molecules comprising functionally inactivated RBS provided herein (for example, as in Section 5.1.5) provide at least a further advantage in that the terminal repeat sequences of DNA molecules may have no or diminished endogenous promoter and/or transcriptional activity (e.g., the P5 AAV promoter, which shares a homolog sequence with the RBS) once in a host cell when compared with DNA molecules with wild type viral ITR sequences. Transcriptional activity or endogenous promoter activity refers to the ability of hairpin ended DNA molecules to promote transgene expression starting from the folded hairpin overhang sequence (e.g., when these sequences contain one or more transcription start sites (TSSs)). Such transcriptional activity can result from the presence of viral proteins (e.g., Rep proteins or NS1 proteins) expressed by viruses that have infected the same host that has been administered the DNA molecules or from binding of endogenous transcription factors expressed in the host cell. The presence of TSSs and promoter sequences or fragments there of (e.g., the P5 promoter) may confound intended transgene expression in therapeutic applications or influence transgene expression cassettes independent of promoter selection, wherein tight control of (e.g., tissue specific) transgene expression by appropriate control elements (e.g., tissue specific promoters) is highly desirable. In certain embodiments, the presence or level of transcriptional activity and/or endogenous promoter activity arising from the folded hairpin overhang DNA sequence of hairpin ended DNA molecules (referred to as “ITR transcriptional activity”) can be determined by measuring the ability of such sequences to promote transgene expression in a host cell (e.g., by detecting report gene expression, qPCR of mRNA transcripts, western blot, etc.) by hairpin ended DNA molecules provided herein that lack a cis-regulatory element (e.g., promoter as described in Section 5.1.1(c)) upstream of the ORF (e.g., by deleting or inactivating the promoter sequence of an expression cassette as described in Section 5.1.1(c)).

In certain embodiments, ITR transcriptional activity can be determined by measuring the residual ability of hairpin ended DNA molecules comprising an expression cassette comprising a tissue specific cis-regulatory element (e.g., tissue specific promoter as described in Section 5.1.1(c)) to promote transgene expression in a host cell not derived from said tissue (e.g., by detecting report genes expression, qPCR of mRNA transcripts, western blot, etc.).

Based on the determination of the reduction ITR transcriptional activity and the ITR transcriptional activity levels, in certain embodiments of the DNA molecules provided herein including in this Section 5.1.5, the ITR transcriptional activity of the DNA molecules when administered to a host is lower than control DNA molecules with wild type viral ITRs and/or RABS by 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, or 20%. In certain embodiments, the ITR transcriptional activity of the DNA molecules when administered to a host is lower than control DNA molecules with RABS and/or with wild type viral ITRs by at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 79%, at least 78%, at least 77%, at least 76%, at least 75%, at least 74%, at least 73%, at least 72%, at least 71%, at least 70%, at least 69%, at least 68%, at least 67%, at least 66%, at least 65%, at least 64%, at least 63%, at least 62%, at least 61%, at least 60%, at least 59%, at least 58%, at least 57%, at least 56%, at least 55%, at least 54%, at least 53%, at least 52%, at least 51%, at least 50%, at least 49%, at least 48%, at least 47%, at least 46%, at least 45%, at least 44%, at least 43%, at least 42%, at least 41%, at least 40%, at least 39%, at least 38%, at least 37%, at least 36%, at least 35%, at least 34%, at least 33%, at least 32%, at least 31%, at least 30%, at least 29%, at least 28%, at least 27%, at least 26%, at least 25%, at least 24%, at least 23%, at least 22%, at least 21%, or at least 20%. In certain embodiments, the ITR transcriptional activity of the DNA molecules when administered to a host is lower than control DNA molecules with RABS and/or with wild type viral ITRs by about 100%, about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, or about 20%.

In a certain embodiment, the DNA molecules provided herein including Section 5.1.1(c), result in no detectable ITR transcriptional activity (e.g., based on the measurement of transgene expression method Section 5.1.1(c)). In certain embodiments, the DNA molecules provided herein including Section 5.1.1(c) result in ITR transcriptional activity of no more than 0.0001%, no more than 0.001%, no more than 0.01%, no more than 0.1%, no more than 1%, no more than 1.5%, no more than 2%, no more than 2.5%, no more than 3%, no more than 3.5%, no more than 4%, no more than 4.5%, no more than 5%, no more than 5.5%, no more than 6%, no more than 6.5%, no more than 7%, no more than 7.5%, no more than 8%, no more than 8.5%, no more than 9%, no more than 9.5%, or no more than 10% of the ITR transcriptional activity resulted from a reference DNA molecule (e.g., the same DNA molecule but with a wild type RABS and/or with wild type ITR sequence). In certain embodiments, the DNA molecules provided herein including Section 5.1.1(c) result in ITR transcriptional activity of about 0.0001%, about 0.001%, about 0.01%, about 0.1%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% of the ITR transcriptional activity resulted from a reference DNA molecule (e.g., the same DNA molecule but with a wild type RABS and/or with wild type ITR sequence). In a certain embodiments, the DNA molecules provided herein result in ITR transcriptional activity of 0.0001%, 0.001%, 0.01%, 0.1%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the mobilization ITR transcriptional activity from a reference DNA molecule (e.g., the same DNA molecule but with a wild type RABS and/or with wild type ITR sequence). Such percentage of ITR transcriptional activity can be determined by using the transgene expression determined as further described in the preceding paragraphs (including the preceding 2 paragraphs).

The DNA sequences or features excluded in the DNA molecules provided herein can be combined in any way with any of the methods provided herein (including in Sections 3, and 5.2-5.4.2), and any of the DNA molecules provided herein (including Sections 3 and 5.1), and contribute to the functional properties of the DNA molecules as provided herein (including Sections 3 and 5.1.1(e)).

### 5.2 Methods of Amplifying DNA Templates

DNA templates provided herein (see Section 5.1, e.g., single-strand circular DNA molecules, double-strand circular DNA molecules) are amplified (e.g., isothermal amplification, e.g., RCA and MDA) according to the method steps described in this Section (Section 5.2) to produce amplification products, which are further processed to generate the hairpin-ended DNA molecules disclosed herein (see Section 5.1.1). Such amplification assists the production of transfection/transcription-ready DNA molecules.

In certain embodiments, the DNA template is amplified by a non-PCR based amplification method to produce the amplification product. In certain embodiments, the DNA template is amplified by isothermal amplification to produce the amplification product. Isothermal amplification has several advantages over PCR, including the ability to amplify DNA at a constant temperature, faster reaction times, and the ability to amplify DNA in a wide range of sample types, including those that may contain contaminants or inhibitory substances that can interfere with PCR.

Isothermal amplification amplifies nucleic acids at a single temperature, without the need for change of reaction temperatures using a thermal cycler (e.g., PCR-based methods). In certain embodiments, the isothermal amplification comprises rolling cycle amplification (RCA). In certain embodiments, the isothermal amplification comprises multiple displacement amplification (MDA). In certain embodiments, the DNA template is amplified by RCA and/or MDA. In certain embodiments, the DNA template is amplified by RCA and MDA to produce the amplification product.

Rolling cycle amplification (RCA) is an isothermal amplification method that uses a circular DNA molecule as a template for a DNA polymerase, which produces long repeating product strands that serve as amplified copies of the circular sequence. Multiple displacement amplification (MDA) is another isothermal amplification method that primes and extends from a template to produce single-stranded DNA chains, which can be continuously re-primed and copied by strand-displacement synthesis. As DNA synthesis can be continuously primed and extended from many positions in the amplified molecules, without required further rounds of denaturation, the MDA can produce a network of hyper-branched DNA structures (Lizardi et al., Nature Genetics (1998); 19:225-232; Dean et al., Proc. Natl. Acad. Sci. U.S.A. (2002); 99:5261-5266).

In certain embodiments, the DNA template (e.g., circular DNA molecule of Section 5.1) disclosed herein is amplified by RCA to produce long ssDNA strands that comprise repeating copies of the DNA template. Such long ssDNA strands serve as further templates for MDA to produce dsDNA molecules (e.g., amplification products).

Amplification products disclosed herein refer to double-stranded DNA molecules that are produced by amplifying the DNA templates (e.g., RCA and MDA). The presently disclosed amplification products comprise sequences that are identical or reverse complementary to the DNA template. In certain embodiments, the amplification products comprise two or more copies of the sequences that are identical or reverse complementary to the DNA template. In certain embodiments, the amplification products are branched dsDNA molecules.

### 5.2.1 Preparing DNA Templates for Amplification

In certain embodiments, the DNA template can be a circular DNA. In certain embodiments, the DNA template is a single-stranded circular DNA or a double-stranded circular DNA. In certain embodiments, the single-stranded circular DNA can be prepared from a double-stranded circular DNA.

### (a) Single-Stranded Circular DNA

In certain embodiments, the DNA template can be a single-stranded circular DNA. In certain embodiments, the single-stranded circular DNA can be created synthetically, e.g., prior to the amplification. In certain embodiments, the single-stranded circular DNA can be created from linear single-stranded DNA (ssDNA) fragments that are generated synthetically and subsequently ligated or otherwise circularized together to form a circular construct. Non-limiting examples of methods that can be used to generate single-stranded circular DNA and/or ssDNA fragments include chemical synthesis, enzyme synthesis, and bacteria-based synthesis.

In certain embodiments, ssDNA fragments are produced by chemical synthesis. Chemical synthesis of ssDNA can be performed by synthesis of oligonucleotides. Synthesis of such oligonucleotides can be performed by one or more phosphoramidite chemistry methods known in the art, using either traditional column-based synthesizers and/or microarray-based synthesizers. In certain embodiments, oligonucleotides for use in producing the ssDNA fragments provided in the present disclosure can yield a ssDNA fragment having a length ranging from about 5 nucleotides (nt) to about 600 nt, from about 10 nt to about 500 nt, from about 15 nt to about 400 nt, from about 20 nt to about 300 nt, from about 25 nt to about 200 nt, or from about 30 nt to about 100 nt. In certain embodiments, oligonucleotides for use in producing the ssDNA fragments of the present disclosure can yield a ssDNA fragment having a length of about 5 nt, about 10 nt, about 15 nt, about 20 nt, about 25 nt, about 30 nt, about 35 nt, about 40 nt, about 45 nt, about 50 nt, about 55 nt, about 60 nt, about 65 nt, about 70 nt, about 75 nt, about 80 nt, about 85 nt, about 90 nt, about 95 nt, about 100 nt, about 150 nt, about 200 nt, about 250 nt, about 300 nt, about 350 nt, about 400 nt, about 450 nt, about 500 nt, about 550 nt, or about 600 nt.

Methods of circularizing a linear ssDNA fragment can be performed using any suitable approach known in the art. In certain embodiments, circularizing a linear ssDNA fragment can be performed by ligating the two ends of the linear ssDNA fragment to each other using a suitable ligase, e.g., a ligase suitable for blunt end ligation (e.g., T4 ligase, T3 ligase, Taq DNA ligase) or sticky end ligation (e.g., T4 ligase, T3 ligase, T7 ligase). Blunt end ligation can be employed by providing a blunt end at one end of the linear ssDNA fragment and a blunt end at the other end of the linear ssDNA fragment. Sticky end ligation can be employed by providing a sticky end at one end of the linear ssDNA fragment and a complementary sticky end at the other end of the linear ssDNA fragment. Typically, circularizing a linear ssDNA fragment can be performed by ligating the two ends of the linear ssDNA fragment to each other using a T4 ligase or a variant thereof. In certain embodiments, circularizing the linear ssDNA fragment provided herein can be attained by splint ligation. In certain embodiments, the circularized DNA may be produced from a linear ssDNA fragment that includes a first sequence at a first end and a second sequence at the end opposite the first end, where circularization is achieved using a splint oligonucleotide that includes sequences complementary to the first and second sequences. According to certain embodiments, a Gibson assembly approach or modified version thereof (e.g., NEBuilder Hifi DNA assembly) is used to join the ends of the linear nucleic acid using the splint oligonucleotide. In certain embodiments, a splint oligonucleotide can be a single-stranded multimer of nucleotides from about 5 nt to about 500 nt, about 5 nt to about 100 nt, or about 5 nt to about 50 nt in length. In certain embodiments, splint oligonucleotides may be about 5 nt, about 10 nt, about 20 nt, about 30 nt, about 40 nt, about 50 nt, about 60 nt, about 70 nt, about 80 nt, about 90 nt, about 100 nt, about 110 nt, about 120 nt, about 130 nt, about 140 nt, about 150 nt, about 160 nt, about 170 nt, about 180 nt, about 190 nt, about 200 nt, about 250 nt, about 300 nt, about 350 nt, about 400 nt, about 450 nt, or about 500 nt in length.

In certain embodiments, a single-stranded circular DNA template provided herein can be created from purified linear ssDNA fragments that are ligated or otherwise circularized together to form a circular construct. Typically, purified linear ssDNA fragments provided herein can be derived from a double-stranded species of DNA (e.g., plasmid DNA, genomic DNA). In certain embodiments, a single-stranded circular DNA template can be prepared from a double-stranded circular DNA. Methods of preparing and purifying linear ssDNA fragments from double-stranded DNA (dsDNA) are described in detail below.

### (b) Double-Stranded Circular DNA

In certain embodiments, the DNA template provided herein is a double-stranded circular DNA. In certain embodiments, the DNA template can be a double-stranded circular DNA plasmid generated using standard molecular biology methods know in the art (See, e.g., Sambrook J. 1991. MOLECULAR CLONING: A LABORATORY MANUAL. New York: Cold Spring Harbor).

Where a double-stranded circular DNA is used as the DNA template herein, the double-stranded circular DNA is denatured prior to performing the amplification in order to form single-stranded circular DNA. Denaturation of the double-stranded circular DNA template can employ any method known in the art for breaking the hydrogen bonds that hold the two DNA strands together. Non-limiting examples of methods of DNA denaturation include heating the double-stranded DNA (dsDNA) to its melting temperature (Tm), incubating dsDNA in an organic solvent (e.g., DMSO), increasing the salt concentration, and subjecting dsDNA to a high pH.

In certain embodiments, the dsDNA templates are denatured at a temperature of at least 70° C., at least 71° C., at least 72° C., at least 73° C., at least 74° C., at least 75° C., at least 76° C., at least 77° C., at least 78° C., at least 79° C., at least 80° C., at least 81° C., at least 82° C., at least 83° C., at least 84° C., at least 85° C., at least 86° C., at least 87° C., at least 88° C., at least 89° C., at least 90° C., at least 91° C., at least 92° C., at least 93° C., at least 94° C., or at least 95° C. In certain embodiments, the dsDNA templates are denatured at a temperature of about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., or about 95° C. In certain embodiments, the dsDNA templates are denatured at a temperature of about 90° C.

In certain embodiments, the dsDNA templates are denatured at a pH of at least 10, at least 10.1, at least 10.2, at least 10.3, at least 10.4, at least 10.5, at least 10.6, at least 10.7, at least 10.8, at least 10.9, at least 11, at least 11.1, at least 11.2, at least 11.3, at least 11.4, at least 11.5, at least 11.6, at least 11.7, at least 11.8, at least 11.9, at least 12, at least 12.1, at least 12.2, at least 12.3, at least 12.4, at least 12.5, at least 13, at least 13.5, or at least 14. In certain embodiments, the dsDNA templates are denatured at a pH of about 10, about 10.1, about 10.2, about 10.3, about 10.4, about 10.5, about 10.6, about 10.7, about 10.8, about 10.9, about 11, about 11.1, about 11.2, about 11.3, about 11.4, about 11.5, about 11.6, about 11.7, about 11.8, about 11.9, about 12, about 12.1, about 12.2, about 12.3, about 12.4, about 12.5, about 13, about 13.5, or about 14.

In certain embodiments, the dsDNA templates are denatured at a salt concentration of at least 1 M, at least 1.5 M, at least 2 M, at least 2.5 M, at least 3 M, at least 3.5 M, or at least 4 M of salt. In some embodiments, the DNA molecules are denatured at a salt concentration of about 1 M, about 1.5 M, about 2 M, about 2.5 M, about 3 M, about 3.5 M, or about 4 M of salt.

In certain embodiments, the dsDNA templates are subject to the denaturing condition for at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 minutes. In certain embodiments, the dsDNA templates are subject to the denaturing condition for about 1, about 2, about 3, about 4, about 5, about 6, about 7, about 8, about 9, about 10, about 11, about 12, about 13, about 14, about 15, about 16, about 17, about 18, about 19, or about 20 minutes. In certain embodiments, the dsDNA templates can be denatured by any combination of denaturing conditions and duration of denaturing as provided herein.

In certain embodiments, the dsDNA templates are denatured prior to adding DNA polymerase to the reaction mixture. In certain embodiments, the dsDNA templates are denatured after adding the primers to the reaction mixture.

In certain embodiments, the DNA templates disclosed herein (e.g., double-stranded or single-stranded circular DNA molecules) are methylated at the MSRE or MSNE sites (Sections 5.1.3(a) and 5.1.4(a)) using an E. coli-based method, where the DNA templates are synthesized and methylated in E. coli. In certain embodiments, the DNA templates are methylated using a cell-free system. In certain embodiments, the methylation is a 4-methylcytosine, a 5-methylcytosine, a 5-hydroxymethylcytosine, a 6-methyladenine, or a combination thereof.

### 5.2.2 Conditions for Producing Amplification Products

DNA templates provided herein are amplified under conditions suitable for nucleic acid amplification (e.g., isothermal amplification, e.g., RCA and MDA) to produce amplification products.

### (a) Polymerase

In certain embodiments, the DNA template disclosed herein (see Section 5.1) is incubated with a DNA polymerase capable of isothermal amplification. In certain embodiments, the DNA template disclosed herein (see Section 5.1) is incubated with a DNA polymerase capable of RCA and/or MDA.

Any suitable DNA polymerase from any polymerase family can be used with the present disclosure, including any commercially available DNA polymerase. In certain embodiments, two, three, four, five or more different DNA polymerases may be used. In certain embodiments, at least one DNA polymerase in the reaction mixture has a proofreading function and at least one DNA polymerase in the reaction mixture does not. DNA polymerases having different replication mechanisms may be used together in the same reaction, e.g., strand displacement types and non-strand displacement types.

In certain embodiments, the DNA polymerase is a strand displacement-type polymerase. In certain embodiments, the strand displacement-type polymerase is Phi29, Deep Vent, Bst DNA polymerase I, or variants of any thereof. The amplification reaction initiates when a primer or the 3′ free end of a single stranded template anneals to a complementary sequence on the DNA template. During DNA synthesis, if it encounters a further primer or other strand annealed to the template, the polymerase displaces and continues its strand elongation. The strand displacement can release single stranded DNA, which can serve as the template for additional priming events. The priming of the newly released DNA can lead to hyper branching, which can be reduced by MSRE and MSNE disclosed herein. Unlike PCR-based methods, strand displacement amplification methods do not require cycles of denaturation for efficient DNA amplification, as double-stranded DNA does not prohibit continued synthesis of new DNA strands. Strand displacement amplification only requires one initial round of heating, to denature the initial template if it is double stranded, to allow the primer to anneal to the primer binding site. Such amplification is isothermal, since no further heating or cooling is required.

In certain embodiments, the strand displacement-type polymerase has a processivity of at least about 20 kb, at least about 30 kb, at least about 50 kb, at least about 70 kb or more. In certain embodiments, the strand displacement-type polymerase has a processivity that is comparable to, or greater than phi29 DNA polymerase.

Suitable polymerases for use with the present disclosure include, but are not limited to, phi29 DNA polymerase, vent exo-DNA polymerase, Bst DNA polymerase, M2 polymerase, or modified versions thereof. In certain embodiments, the polymerase is a modified M2 polymerase. Additional polymerases suitable for the methods disclosed herein are disclosed in U.S. Pat. No. 10,934,533 and WO2023283092, the content of each of which is incorporated by reference herein. Template-independent polymerases may be used, such as terminal transferases.

In certain embodiments, the polymerase is a polymerase of Phi29 family. In certain embodiments, the polymerase is a polymerase from bacteriophage Phi29, B103, M2 (Y), or Nf. These polymerases are suitable for RCA and MDA as they do not need accessory proteins and possess a number of distinctive biochemical properties including a strong binding capacity for single stranded DNA, strand displacement activity, high processivity, and a proofreading activity.

In certain embodiments, the polymerase is highly stable, such that its activity is not substantially reduced by prolonged incubation under process conditions. In certain embodiments, the polymerase has a long half-life under a range of process conditions including but not limited to temperature and pH. In certain embodiments, the polymerase has one or more characteristics suitable for a manufacturing process. In certain embodiments, the polymerase has high fidelity, for example through having proofreading activity. In certain embodiments, the polymerase displays one or more of: high processivity, high strand-displacement activity, and a low Kₘ for dNTPs and DNA. In certain embodiments, the polymerase can use circular and/or linear DNA as templates. In certain embodiments, the polymerase can use dsDNA or ssDNA as templates. In certain embodiments, the polymerase does not display DNA exonuclease activity that is not related to its proofreading activity. In certain embodiments, the polymerase can use an alternative nucleic acid as a template.

In certain embodiments, the polymerase is a wildtype polymerase. In certain embodiments, the polymerase is a mutated polymerase, wherein the mutated polymerase comprises one or more amino acid modifications (e.g., deletion, substitution, and/or addition) relative to the wildtype polymerase. In certain embodiments, the polymerase is a Phi mutant. Suitable Phi mutants that can be used with the methods disclosed herein are disclosed in International Patent Publication No. WO2021163052, which is incorporated by reference herein. In certain embodiments, the polymerase is a M2 mutant. Suitable M2 mutants that can be used with the methods disclosed herein are disclosed in U.S. Pat. No. 10,934,533, which is incorporated by reference herein. In certain embodiments, the polymerase is a Phi29 mutant. Suitable Phi29 mutants that can be used with the methods disclosed herein are disclosed in International Patent Publication No. WO2019019222, which is incorporated by reference herein.

In certain embodiments, the polymerase is a fusion protein construct comprising a SSB Protein to enhance ssDNA binding and stability.

Template DNA and polymerase are incubated in a suitable buffer, which can be chosen based on the nature of the polymerase.

### (b) Primers for Initiating Amplification and Producing Amplification Products

In certain embodiments, the DNA template disclosed herein (see Section 5.1) is further incubated with primer(s) for amplification of DNA templates and for producing of amplification products (e.g., double-stranded linear DNA molecules). Binding sites for sequence-specific primers are described in Section 5.1.2. Suitable primers based on these binding sites are incubated with the DNA template and the polymerase. In certain embodiments, the primer(s) consist entirely of a complementary sequence to the primer binding site sequence. In certain embodiments, primer(s) can comprise additional, non-complementary sequences or other modifications at the 5′ end.

In certain embodiments, site-specific primers can be used with the methods and compositions provided herein. Random primers can also be used (such as random hexamers). In certain embodiments, the use of sequence-specific primers is preferred over the use of random primers.

The primer binding sites described in this section are for site-specific primers. In certain embodiments, site specific primers are provided as a primer pair. One primer in such a primer pair is complementary to, and thus hybridizes to, one strand of the template thereby initiating synthesis of the complementary strand of DNA; the other primer is complementary to, and thus hybridizes to, that newly-synthesized strand of DNA thereby initiating the synthesis of the second strand of DNA resulting in the synthesis of double-stranded DNA, e.g., the amplification products. The DNA template can be single-stranded or double-stranded. In certain embodiments, multiple site-specific primers can be used for the synthesis of the first strand and/or for the synthesis of the second strand. The sections below describe the primers that can be used with the methods and compositions provided herein primarily for a primer pair of a first primer and a second primer that are designed to result in the synthesis of double stranded DNA from either a single stranded or a double stranded template.

In certain embodiments, more than two primers are used with the present disclosure. The skilled artisan would know, however, based in this guidance how to design primers if more than two primers are being used.

In certain embodiments, the primers are independent from each other to avoid the formation of primer dimers.

In certain embodiments, the primers are DNA molecules, RNA molecules, or nucleic acid molecules having DNA or RNA characteristics. In certain embodiments, the primers are resistant to exonuclease digestion. In certain embodiments, the primers comprise two or more nucleotides (e.g., three or more nucleotides) that are linked by a phosphorothioate linkage, and thus are resistant to exonuclease digestion.

In certain embodiments, the primers comprise modified nucleotides. In certain embodiments, the nucleotide-modified primers (e.g., RNA/2′-O-methyl RNA chimeric primers) have a higher melting temperature (Tm) than DNA primers. Such features increase the stability of primer hybridization and increase strand invasion by the primers, which leads to more efficient priming.

In certain embodiments, the primers are chimeric primers, which comprise at least two types of nucleotides. In certain embodiments, the chimeric primers comprise deoxyribonucleotides and ribonucleotides, ribonucleotides and modified nucleotides, or two different types of modified nucleotides. In certain embodiments, the chimeric primers are peptide nucleic acid/nucleic acid primers (e.g., 5′-PNA-DNA-3′ and 5′-PNA-RNA-3′ primers). Such peptide nucleic acid/nucleic acid primers can be used for more efficient strand invasion and polymerization invasion. In certain embodiments, the DNA and RNA portions of such primers comprise random or degenerate sequences. Additional exemplary forms of chimeric primers include, for example, 5′-(2′-O-Methyl)RNA-RNA-3′ or 5′-(2′-O-Methyl)RNA-DNA-3′.

Modified nucleotides, such as nucleotide analogs, are known in the art and can be used with the presently disclosed primers. A nucleotide analog is a nucleotide which comprises modification to either the base, sugar, or phosphate moieties. Modifications to the base moiety include natural and synthetic modifications of A, C, G and T/U as well as different purine or pyrimidine bases, such as uracil-5-yl, hypoxanthin-9-yl (I)₅ and 2-aminoadenin-9-yl. Exemplary modified bases that can be used with the present disclosure include but are not limited to 5-methylcytosine (5-mC), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional exemplary base modifications that can be used with the present disclosure are disclosed in U.S. Pat. No. 3,687,808; Englisch et al. (1991) ANGEWANDTE CHEMIE, International Edition 30, 613; and Sanghvi, Y. S., Chapter 15, ANTISENSE RESEARCH AND APPLICATIONS, pages 289-302, Crooke, S. T. and Lebleu, B. ed., CRC Press, 1993, the contents of which are incorporated by reference in their entireties. Nucleotide analogs, such as 5-substituted pyrimidines, 6-azapyrimidines and N-2, N-6 and O-6 substituted purines, including 2-aminopropyladenine, 5-propynyluracil, 5-propynylcytosine, and 5-methylcytosine can increase the stability of duplex formation. In certain embodiments, the modified bases function as universal bases. Exemplary universal bases include 3-nitropyrrole and 5-nitroindole. Universal bases can substitute for normal bases but have no bias in base pairing and can base pair with any other base. Primers comprising nucleotides with universal bases are useful for reducing or eliminating amplification bias against repeated sequences in a target sample. In certain embodiments, base modifications are combined with for example a sugar modification, such as 2′-O-methoxyethyl, to achieve properties such as increased duplex stability. Exemplary base modifications are described in U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, the contents of which are incorporated by reference in their entireties.

In certain embodiments, nucleotide analogs comprise modified sugar moiety. In certain embodiments, the modified sugar moiety comprises a natural and/or synthetic modification to the ribose and/or deoxyribose. Exemplary sugar modifications include but are not limited to the modifications at the 2′ position, including OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10, alkyl or C2 to C10 alkenyl and alkynyl. Exemplary 2′ sugar modifications also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙCH3)₂ where n and m are from 1 to about 10. Additional exemplary modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂, CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications can be made at other positions on the sugar, such as the 3′ position of the sugar on the 3′ terminal nucleotide or in 2′-5′ linked oligonucleotides and the 5′ position of 5′ terminal nucleotide. Exemplary modified sugars also include those that contain modifications at the bridging ring oxygen, such as CH₂ and S. Exemplary nucleotide sugar analogs include sugar mimetics such as cyclobutyl moieties in place of the pentofuranosyl sugar. Preparation of such modified sugar structures is described in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

In certain embodiments, nucleotide analogs comprise modified phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkyl-phosphonates, thionoalkylphosphotriesters, and boranophosphates. The phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Methods of making and use of nucleotides containing modified phosphates are described in U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference in its entirety. It is understood that nucleotide analogs disclosed herein can comprise a single modification, or multiple modifications within one of the moieties or between different moieties.

Nucleotide substitutes are nucleotides or nucleotide analogs having phosphate moiety and/or sugar moieties replaced. In certain embodiments, the nucleotide substitutes are molecules having similar functional properties to nucleotides, but do not contain a phosphate moiety, such as peptide nucleic acid (PNA). In certain embodiments, the nucleotide substitutes include molecules that recognize and hybridize to complementary nucleic acids in a Watson-Crick or Hoogsteen manner, but are linked together through a moiety other than a phosphate moiety. In certain embodiments, the nucleotide substitutes conform to a double helix type structure when interacting with the appropriate target nucleic acid.

Substitutes for the phosphate include short chain alkyl or cycloalkyl internucleoside linkages, mixed heteroatom and alkyl or cycloalkyl internucleoside linkages, or one or more short chain heteroatomic or heterocyclic internucleoside linkages. Substitutes for the phosphate further include morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Methods of making and use of phosphate replacements are described in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference in its entirety.

In certain embodiments, a nucleotide substitute that comprises both the sugar and the phosphate moieties of the nucleotide can be replaced, by for example an amide type linkage (aminoethylglycine) (PNA). U.S. Pat. Nos. 5,539,082; 5,714,331; and 5,719,262 describe how to make and use PNA molecules (see also Nielsen et al. (1991) Science 254, 1497-1500), each of which is herein incorporated by reference in its entirety.

In certain embodiments, primers disclosed herein comprise different or the same types of nucleotides. In certain embodiments, the primers comprise at least one ribonucleotide, at least one 2′-O-methyl ribonucleotide, or a mixture thereof. In some embodiments, primers disclosed herein are comprised of about 10% to about 50%, about 50% or more, or 100% ribonucleotides, 2′-O-methyl ribonucleotides, or a mixture of thereof. In certain embodiments, the nucleotides comprise different types of bases. In certain embodiments, the nucleotides comprise universal bases, such as 3-nitropyrrole or 5-nitroindole universal bases. In some embodiments, primers disclosed herein are comprised of about 10% to about 50%, about 50% or more, or 100% universal bases.

In certain embodiments, the primer comprises a non-complementary portion, which includes an additional sequence at the 5′ end of the primer that is not complementary to the primer binding site. In certain embodiments, the non-complementary portion of the primer can facilitate strand displacement during DNA replication. In certain embodiments, the non-complementary portion is from 1 to 100 nucleotides long. In certain embodiments, the non-complementary portion is from preferably from 3 to 10 nucleotides long.

The size of the primers can be, independently from each other, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 60, 65, 70, 75, 80, 85, 90, 95, 100 or more nucleotides to allow production of an amplification product. In certain embodiments, the size of the primers is, independently from each other, up to 10, up to 25, up to 30, up to 35, up to 40, up to 45, up to 50, up to 55, up to 60, up to 65, up to 70, up to 75, up to 75, up to 80, up to 85, up to 90, up to 95, or up to 100 nucleotides long. In certain embodiments, the primers have about, i.e., +/−10%, the same length. In certain embodiments, the primers have the same length.

In certain embodiments, the GC content of the primers are, independently from each other, at least 30%, at least 35%, at least 40%, at least 45%, at least 50%, at least 55%, at least 60%, at least 65%, at least 70%, at least 75%, or at least 80%. In certain embodiments, the GC content of the primers are, independently from each other, at most 30%, at most 35%, at most 40%, at most 45%, at most 50%, at most 55%, at most 60%, at most 65%, at most 70%, at most 75%, or at most 80%.

In certain embodiments, the sequence of the primer is independently at least 80%, 85%, 90%, 95%, or is 100% complementary to the template strand and thus to their respective primer binding sites.

In certain embodiments, the 3′ end of a primer and thus the 5′ end of the respective primer binding site to be used with the methods and compositions provided herein is a Guanosine (“G”) or a Cytosine (“C”), two G/Cs, or three G/Cs.

Melting temperatures of primers depend on G/C contents, size of the sequences, and composition of the primers. Melting temperatures of the primers disclosed herein can be adjusted to minimize and eliminate non-specific bindings of the primers.

In certain embodiments, the binding site of the primer is located outside the inverted repeat flanked sequence of interest. In other words, the primer binding site is located on the opposite side of both inverted repeats relative to the sequence of interest. In certain embodiments, the primer binding site is not in the inverted repeat and/or is not in the sequence of interest. In certain embodiments, the primer binding sites for the primer pair are flanking each inverted repeat in a way that the 3′ end of the first and the second primers point towards the inverted repeats and the sequence of interest.

In certain embodiments, the binding site for the first primer and for the second primer are at different locations in the DNA template. The distance between the two primer binding sites can vary at a wide range, such that the location of the first primer binding site is independent from the second primer binding site. In certain embodiments, the primers are at least 10, 50, 100, 150, 200, 250, 500, 750, 1000, 1500, 2000, 2500, or at least 5000 nucleotides apart from each other. In certain embodiments, the two primers are at most 10, 50, 100, 150, 200, 250, 500, 750, 1000, 1500, 2000, 2500, or at most 5000 nucleotides apart from each other. The distance between the primer binding sites and the downstream restriction enzyme sites, MSRE sites, and MSNE sites, which are located outside the segment comprising the inverted repeat-flanked sequence of interest, can also vary at a wide range. In certain embodiments, the binding site of the first primer and/or the second primer is located inside the segment comprising the inverted repeat-flanked sequence of interest. In certain embodiments, the binding site is located in ITR, backbone sequence, or the sequence of interest.

In certain embodiments, the binding site is not in the MSRE or MSNE site. In certain embodiments, the binding site is in the MSRE or MSNE site, and the primers are modified to allow protection of the formed double strand upon primer binding.

In certain embodiments, the primers incubated with the DNA template are a set of random primers. The random primers randomly prime the DNA template and/or the ssDNA strands amplified therefrom. The random primers in the set are collectively, and randomly, complementary to nucleic acid sequences distributed throughout the DNA template and the ssDNA strands amplified therefrom. Amplification proceeds by replication initiating at each primer and continuing so that the growing strands encounter and displace adjacent replicated strands. In certain embodiments, the size of the random primers is at least 15 nucleotides long. In certain embodiments, the size of the random primers is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 60, 65, 70, 75, 80, 85, 90, 95, or 100 nucleotides long.

In certain embodiments, the molar ratio between primer and template is at least 1:1; 5:1; 10:1; 100:1; 1,000:1; 2,000:1; 3,000:1; 4,000:1; 5,000:1; 10,000:1; or at least 20,000:1 (i.e., at least 1, 5, 10, 100, or at least 1,000, 2,000, 3,000, 4,000, 5,000, 10,000, or at least 20,000 molecules of each primer per molecule of template). In certain embodiments, the first primer (for the synthesis of the first strand of DNA) is added at the same molar ratio to the template as the second primer (for the synthesis of the second strand to generate double stranded DNA). In certain embodiments, the second primer is added at a higher ratio than the first primer. In certain embodiments, the second primer is added at an at least 2-fold; 5-fold; 10-fold; 50-fold; 100-fold; 500-fold; or at least 1000-fold higher excess to the template as compared to the first primer. For example, the first primer is added at a ratio of 10:1 (i.e., ten molecules of first primer per molecule of template) and the second primer is added at 10-fold higher excess, i.e., at a ratio of 100:1 (i.e., hundred molecules of second primer per molecule of template).

In the case of a primer pair (one for each of the two strands of the to-be-synthesized double stranded DNA, i.e., amplification product), both primers are added to the amplification reaction together at the beginning of the reaction. In certain embodiments, a first primer is added first to initiate the synthesis of a first strand of DNA. The second primer is added later on in the reaction to initiate the synthesis of the second strand. In certain embodiments, the second primer is added after at least 2, 3, 4, 5, 6, 7, 8, 9, 10, 15, 20, 25, 30, 35, 40, 45, or at least 50 single stranded copies of the template have been synthesized.

In certain embodiments, the primers are RNA primers. In certain embodiments, the RNA primers are used for the initiation of DNA replication. In certain embodiments, the RNA primers are used with M2 polymerase for amplification of the DNA template.

In certain embodiments, the primers are phosphate-based DNA, i.e., the primers do not comprise phosphorothioates or other chemically modified nucleotides.

(c) dNTPS

Any suitable nucleotide may be used with the present disclosure for amplifying a DNA template. In certain embodiments, the nitrogenous bases are adenine (A), guanine (G), thymine (T), cytosine (C), and uracil (U). In certain embodiments, the nitrogenous bases comprise modified bases, such as 5-methylcytosine (m5C), pseudouridine (Y), dihydrouridine (D), inosine (I), and 7-methylguanosine (m7G). In certain embodiments, the nitrogenous bases comprise artificial bases. In certain embodiments, a five-carbon sugar is a deoxyribose, such that the nucleotide is a deoxynucleotide.

In certain embodiments, the nucleotides are deoxynucleoside triphosphate (dNTP). In certain embodiments, the dNTPs are unmodified deoxynucleoside triphosphates (dNTPs), alpha phosphate modified dNTPs, sugar modified dNTPs, base modified dNTPs, and/or labeled dNTPS (e.g., biotin-labeled dNTPs). Suitable dNTPs that can be used with the present disclosure include dATP (deoxyadenosine triphosphate), dGTP (deoxyguanosine triphosphate), dTTP (deoxythymidine triphosphate), dUTP (deoxyuridine triphosphate), dCTP (deoxycytidine triphosphate), dITP (deoxyinosine triphosphate), dXTP (deoxyxanthosine triphosphate), and derivatives and modifications thereof. In certain embodiments, the dNTPs comprise one or more of dATP, dGTP, dTTP, dCTP, modifications thereof, and/or derivatives thereof. In certain embodiments, the dNTPs comprise a mixture of dATP, dGTP, dTTP, and dCTP, or modifications thereof. Any suitable ratios of these dNTPs can be used, according to the needs of the reaction.

In certain embodiments, the nucleotide complexes are added to the reaction mixture prior to the addition of nucleotidyltransferase. In certain embodiments, the nucleotide complexes comprise modified nucleotides. In certain embodiments, the nucleotide complexes are supplied as a mixture of one or more suitable bases, e.g., one or more (e.g., two, three or four) of adenine (A), guanine (G), thymine (T), and/or cytosine (C). In certain embodiments, the nucleotides are natural nucleotides (i.e., unmodified nucleotides), or modified nucleotides that act like natural nucleotides and are biologically active (i.e., LNA nucleotides-locked nucleic acid), or modified and biologically inactive nucleotides, or a mixture of unmodified and modified nucleotides, or a mixture of biologically active and biologically inactive nucleotides. In certain embodiments, the nucleotides used in the amplification reaction comprise modified nucleotides (e.g., modified nucleotides disclosed in paragraphs [0332]-[0337] of the present disclosure).

In certain embodiments, the nucleotides are associated with one or more types of counter ion (e.g., a mixture of divalent and monovalent cations) and form a nucleotide complex in the reaction mixture. In certain embodiments, the monovalent cations are metal ions or a polyatomic ions, such as an oxonium ion. In certain embodiments, the divalent cations are metal ions or polyatomic ions. In certain embodiments, the monovalent cations are metal ions. Exemplary metal ions that can be used with present disclosure include but are not limited to alkali metals, such as lithium (Li⁺), sodium (Na⁺), potassium (K⁺), rubidium (Rb⁺), caesium (Cs⁺) or francium (Fr⁺); and transition metals such as copper (Cu⁺), silver (Ag⁺), gold (Au⁺) or roentgenium (Rg⁺). In certain embodiments, the nucleotides are associated with lithium (Li⁺), sodium (Na⁺), potassium (K⁺), rubidium (Rb⁺), caesium (Cs⁺), and/or francium (Fr⁺).

In certain embodiments, the monovalent cations are polyatomic ions. In certain embodiments, the monovalent cations are oxonium ions. Exemplary oxonium ions include hydronium ion (H₃O⁺), ammonium (NH₄₊), and ionic derivatives thereof (e.g., monoalkyl ammonium, dialkyl ammonium, trialkyl ammonium, choline, quaternary ammonium and imidazolium).

In certain embodiments, the divalent cation is a metal ion, such as Mg²⁺, Be²⁺, Ca²⁺, Sr²⁺, Mn²⁺ or Zn²⁺. In certain embodiments, the divalent cation is Mg²⁺ or Mn²⁺. In certain embodiments, the ratio between the divalent metal cations and the nucleotide (nucleotide ion or nucleotide ionic species) is between 0.2:1 and 2:1, between 0.5:1 and 1.5:1, or about 1:1 in the reaction mixture. Ratios lower than 1:1 are desirable and are preferable in DNA synthesis since ratios higher than 1:1 may lead to some infidelity in DNA synthesis.

Between 0.2 and 2 divalent cations may be associated with the nucleotide complex. This range includes 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2 divalent cations per nucleotide complex. Those skilled in the art will appreciate the non-whole numbers represent a sharing of the divalent ion between nucleotide free acids.

In certain embodiments, sodium, potassium or ammonium cation or a mixture of sodium, potassium ammonium cations are associated with the nucleotide complex (dNTP complex). In certain embodiments, the magnesium:dNTP complex concentration ratio varies based on the counter ion in the nucleotide complex. In certain embodiments, the magnesium:dNTP complex concentration ratio is at least 0.5:1, at least 0.6:1, at least 0.7:1, at least 0.8:1, at least 0.9:1, at least 1.0:1, at least 1.1:1, at least 1.2:1, at least 1.3:1, at least 1.4:1, at least 1.5:1, at least 1.6:1, at least 1.7:1, at least 1.8:1, at least 1.9:1, or at least 2.0:1. In certain embodiments, preferred magnesium:dNTP complex concentration ratios when a sodium, potassium or ammonium dNTP complex or a mixture thereof is used are in the range of 0.8:1-1.5:1.

In certain embodiments, the total nucleotides or nucleotide complexes, including the sum of each type of nucleotide, are present at a concentration of at least 1 mM at the start of the reaction. It will be understood that nucleotides supplied as complexes may dissociate in water and other solvents to form an anionic nucleotide entity (nucleotide ion, nucleotide ionic species) and the associated cations.

### (d) Additional Enzymes

Additional proteins can be included in the reaction mixture to facilitate the amplification reaction. In certain embodiments, the DNA template is incubated with at least one pyrophosphatase. In certain embodiments, the pyrophosphatase is a yeast inorganic pyrophosphatase. In certain embodiments, the DNA template is incubated with two, three, four, five or more different pyrophosphatases. The pyrophosphatases can degrade pyrophosphate that is produced by DNA polymerase from dNTPs during strand replication. Buildup of pyrophosphate in the reaction mixture can inhibit the activity of DNA polymerases and reduce the speed and efficiency of DNA amplification. Pyrophosphatases can break down pyrophosphate into non-inhibitory phosphate. Any suitable pyrophosphatases known in the art can be used with the presently disclosed subject matter. In certain embodiments, the DNA template is incubated with a fusion construct containing the DNA Polymerase and a pyrophosphatase covalently linked together.

In certain embodiments, the DNA template is incubated with a DNA helicase. DNA helicase can facilitate the separation of double-stranded DNA into single strands, and thus allowing each strand to be replicated. Any suitable helicases known in the art can be used with the presently disclosed subject matter. In certain embodiments, the DNA template is incubated with a fusion construct containing a DNA polymerase and a helicase covalently linked together.

In certain embodiments, the DNA template is incubated with a single stranded DNA binding protein (SSB). During DNA replication, the SSB can wrap single-stranded DNA (ssDNA) with high affinity to protect it from degradation and prevent secondary structure formation. Any suitable SSBs known in the art can be used with the presently disclosed subject matter. In certain embodiments, the DNA template is incubated with a fusion construct containing a helicase and a SSB protein covalently linked together.

In certain embodiments, the DNA template is incubated with a PrimPol. PrimPol is a eukaryotic protein with both DNA polymerase and DNA Primase activities. PrimPol can initiate replication without the need of an RNA primer and can extend from primers produced by PrimPol. Any suitable PrimPols known in the art can be used with the presently disclosed subject matter.

### (e) Amplification Reaction

The amplification reaction employs conditions that promote annealing of primers to the template. Such conditions include providing a single-stranded nucleic acid allowing for hybridization of the primers, and/or using a temperature and buffer that allow for annealing of the primer to the template. Appropriate annealing/hybridization conditions may be selected depending on the nature of the primer(s). In certain embodiments, the annealing is carried out following denaturation using heat followed by gradual cooling to the desired reaction temperature. In certain embodiments, the annealing is carried out by chemical denaturation using denaturing agents such NaOH or KOH, which can elevate the pH followed by reduction of the pH below denaturation conditions.

An appropriate temperature for the amplification reaction disclosed herein is selected based on the temperature at which a specific polymerase has optimal activity. In certain embodiments, the amplification reaction uses a phi29 DNA polymerase, and the reaction temperature is from about 25 to about 35, or about 30 degrees centigrade (° C.). A thermostable phi29 may operate at a higher constant temperature than the phi29 DNA polymerase. The skilled person would be able to identify a suitable temperature for efficient amplification. For example, the amplification reaction could be carried out at a range of temperatures, and yields of amplified DNA are monitored to identify an optimal temperature range for a given polymerase. In certain embodiments, the amplification is carried out at a constant temperature and is isothermal.

Suitable buffering agents and pH are also employed for enzyme performance or stability. In certain embodiments, the pH of the reaction mixture is maintained within the range of from 3 to 10, from 5 to 8, or about 7 or 7.5. In certain embodiments, at least one buffering agent (also called pH buffering agent) is used to maintain the pH. Exemplary buffers include but are not limited to MES, Bis-Tris, ADA, ACES, PIPES, MOBS, MOPS, MOPSO, Bis-Tris Propane, BES, TES, HEPES, DIPSO, TAPSO, Trizma, HEPPSO, POPSO, TEA, EPPS, Tricine, Gly-Gly, Bicine, HEPBS, TAPS, AM PD, TABS, AMPSO, CHES, CAPSO, AMP, CAPS, CABS, phosphate, citric acid-sodium hydrogen phosphate, citric acid-sodium citrate, sodium acetate-acetic acid, imidazole and sodium carbonate-sodium bicarbonate. Suitable buffering agents do not provide further cations to the reaction mixture, nor complex with metal cations present in the reaction mixture. A buffering agent can include one or more additional salts composed of a cationic and anionic species (i.e., sodium chloride, potassium chloride) and/or detergents to ensure the optimal activity or stability of the enzymes. A minimal buffer is composed of only a buffering reagent with no additional salts or detergents provided, with the proviso that small amounts of cationic species may be present for DNA synthesis in which chemical denaturation is required.

Approaches other than heat can be used to denature double stranded DNA. Double stranded DNA can be denatured by exposure to a high or low pH environment or where cations are absent or present in very low concentrations, such as in deionized water. The polymerase requires the binding of a short oligonucleotide primer sequence to a single stranded region of the DNA template to initiate its replication. The stability of this interaction and therefore the efficiency of DNA synthesis may particularly be influenced by the concentration of metal cations and particularly divalent cations such as magnesium (Mg²⁺) ions, which may be seen as an integral part of the process.

In certain embodiments, additional divalent metal ions, namely divalent cations that are supplied externally to the nucleotide complex are used in the amplification reaction. In certain embodiments, the additional divalent metal ions include salts of divalent metal ions: magnesium (Mg²⁺), manganese (Mn²⁺), calcium (Ca²⁺), beryllium (Be²⁺), zinc (Zn²⁺) and strontium (Sr²⁺). In certain embodiments, the additional divalent metal ions are magnesium or manganese, which act as a cofactor in DNA synthesis. Any suitable anion may be utilized in such salts, and the effect on the pH of the reaction mixture should be suitably accounted for.

In certain embodiments, the reaction mixture includes detergents. Exemplary suitable detergents include Triton X-100™, Tween 20™, and derivatives thereof. In certain embodiments, the reaction mixture includes stabilizing agents. Exemplary suitable stabilizing agents include bovine serum albumin (BSA), other stabilizing proteins, sugars or sugar derivatives. In certain embodiments, the reaction mixture includes sucrose. Reaction conditions may also be improved by adding agents that relax DNA and ease template denaturation. Such agents include, for example, dimethyl sulphoxide (DMSO), formamide, glycerol and betaine. DNA condensing agents may also be included in the reaction mixture. Such agents include, for example, polyethylene glycol or cationic lipid or cationic polymers.

In certain embodiments, these components may be reduced or removed from the reaction mixture, for example in the minimal or no added buffering agent systems.

The skilled person is able to modify and optimize synthesis conditions disclosed herein using additional components and conditions, and selecting concentrations of particular agents based on knowledge in the art.

Amplification factor disclosed herein is calculated as the ratio between the amount of amplified DNA products at the end of the amplification reaction and the amount of DNA template at the start of the amplification reaction. In certain embodiments, the amplification is terminated after an amplification factor of at least 2, at least 5, at least 10, at least 15, at least 20, at least 30, at least 40, at least 50, at least 60, at least 70, at least 80, at least 90, at least 100, at least 250, at least 500, least 1000, at least 2000, at least 4000, at least 6000, at least 8000, at least 10000, at least 20000, at least 40000, at least 50000, at least 60000, at least 80000, or at least 100000 is reached. In certain embodiments, the amplification is terminated after an amplification factor of at most 2, at most 5, at most 10, at most 15, at most 20, at most 30, at most 40, at most 50, at most 60, at most 70, at most 80, at most 90, at most 100, at most 250, at most 500, at most 750, at most 1000, at most 2000, at most 4000, at most 6000, at most 8000, at most 10000, at most 20000, at most 40000, at most 50000, at most 60000, at most 80000, or at most 100000 is reached.

In certain embodiments, generation of precursors of hairpin-ended DNA molecules from the amplification product (see Section 5.3.1) and/or generation of hairpin-ended DNA from the amplification product (see Section 5.3.2) is initiated after an amplification factor of at least 2, least 5, least 10, least 15, at least 20, at least 30, at least 40, at least 50, at least 60, at least 70, at least 80, at least 90, at least 100, at least 250, at least 500, at least 750, least 1000, at least 2000, at least 4000, at least 6000, at least 8000, at least 10000, at least 20000, at least 40000, at least 50000, at least 60000, at least 80000, or at least 100000 is reached. In certain embodiments, generation of precursors of hairpin-ended DNA molecules from the amplification product and/or generation of hairpin-ended DNA from the amplification product is initiated after an amplification factor of at most 2, least 5, at most 10, at most 15, at most 20, at most 30, at most 40, at most 50, at most 60, at most 70, at most 80, at most 90, at most 100, at most 250, at most 500, at most 750, at most 1000, at most 2000, at most 4000, at most 6000, at most 8000, at most 10000, at most 20000, at most 40000, at most 50000, at most 60000, at most 80000, or at most 100000 is reached. In certain embodiments, the amplification is still ongoing while generation of precursors of hairpin-ended DNA molecules and/or hairpin-ended DNA is initiated. In certain embodiments, the amplification has been terminated at the time the generation of precursors of hairpin-ended DNA molecules and/or hairpin-ended DNA is initiated.

In certain embodiments, the amplification reaction is carried out in minimal buffering agents, where no additional salts that have been shown to enhance DNA synthesis or assist in primer binding, or detergents, are added. In certain embodiments, the minimal buffer comprises a buffering agent to stabilize the pH. In certain embodiments, the minimal buffering agent contains cations for chemically denaturing the template, such as sodium, potassium, or ammonium hydroxide.

As disclosed in Section 5.2.4, inclusion of MSRE or MSNE during the amplification reaction allows the continuous digestion of nascent amplification products, which reduces the viscosity, improves fidelity, and reduces extensive amplification of already amplified product.

In certain embodiments, the amplification reaction is in an industrial scale, for example, scaled up into several or tens of liters. In certain embodiments, the amplification reaction allows the efficient incorporation of nucleotides into the DNA product.

### (f) Characteristics of Amplification Products

In certain embodiments, the amplification product has the following properties at the time the amplification reaction is terminated and/or at the time precursors of hairpin-ended DNA molecules and/or hairpin-ended DNA is initiated. These properties can be adjusted by the use of an MSRE or an MSNE as described in Section 5.2.4. Amplification products as disclosed herein, especially with the use of MSRE or MSNE, have low viscosity and high fidelity, which make them suitable for subsequence processing into transfection/transcription-ready DNA molecules.

In certain embodiments, the viscosity of a population of amplification products after the amplification reaction disclosed herein is at most 500 millipascal-seconds (mPa·s), at most 1000 mPa·s, at most 2500 mPa·s, at most 5000 mPa's, at most 10,000 mPa·s, at most 20,000 mPa·s, at most 25,000 mPa·s, at most 30,000 mPa's, at most 40,000 mPa·s, or at most 50,000 mPa·s, where no restriction enzymes or MSRE or MSNE are used during or at the termination of the amplification reaction. In certain embodiments, the viscosity of the amplification products during and after termination is at most 5 mPa's, at most 10 mPa·s, at most 50 mPa·s, at most 100 mPa·s, at most 200 mPa·s, at most 400 mPa·s, at most 600 mPa·s, or at most 1000 mPa·s.

In certain embodiments, the average error rate in a population of amplification products after an amplification reaction disclosed herein is at most 1, at most 2, at most 3, at most 4, at most 5, at most 6, at most 7, at most 8, at most 9, at most 10, at most 15, at most 20, or at most 25 mutations per 1000 nucleotides polymerized. In certain embodiments, the average error rate in a population of amplification product after an amplification reaction disclosed herein is at most 1 error per 10³ nucleotides, at most 1 error per 10⁴ nucleotides, at most 1 error per 10⁵ nucleotides, at most 1 error per 10⁶ nucleotides, at most 2 errors per 10⁶ nucleotides, at most 3 errors per 10⁶ nucleotides, at most 4 errors per 10⁶ nucleotides, at most 5 errors per 10⁶ nucleotides, at most 6 errors per 10⁶ nucleotides, at most 7 errors per 10⁶ nucleotides, at most 8 errors per 10⁶ nucleotides, at most 9 errors per 10⁶ nucleotides, or at most 1 error per 10⁷ nucleotides polymerized. Polymerase errors include, but are not limited to, nucleotide substitutions, nucleotide deletions, and nucleotide insertions identified in the amplification product and/or in the precursors of hairpin-ended DNA molecules and/or in the hairpin-ended DNA molecule that are not present in the circular DNA template. The polymerase error rate can be determined by methods known in the art, such as in vitro forward mutation assays (e.g., lacZ-based assays), gel-based assays (e.g., denaturing gradient gel electrophoresis or DGGE), sanger sequencing (e.g., colony sequencing), or high-throughput assays based on next-generation sequencing (e.g., NGS).

The sequence of interest can have any sequence composition or a sequence composition that amplifies the naturally low DNA polymerase error rates. The sequence of interest can codify a reporter protein, such as LacZ. The amplification product and/or the precursors of hairpin-ended DNA molecules and/or the hairpin-ended DNA molecule can be sequenced using diverse NGS platforms, such as Illumina NGS platforms, Oxford Nanopore Technologies sequencing platforms, or PacBio sequencing platforms. A person skilled in the art will appreciate that DNA molecules require library preparation before NGS and that the preparation differs depending on the sequencing platform. The person skilled in the art will also understand that the amount of sequenced reads is at least as many reads as the inverse of the error rate that is being measured. Unique Molecular Identifiers (also known as UMIs) can be added to the library preparation to identify different reads coming from the same original molecule.

### 5.2.3 Termination of the Amplification Reaction

In certain embodiments, methods disclosed herein further comprise terminating the amplification reaction described in Section 5.2.2. In certain embodiments, the amplification reaction is terminated once the desired amount of amplification products is amplified. In certain embodiments, the amplification is terminated after an amplification factor of at least 2, at least 5, at least 10, at least 15, at least 20, at least 30, at least 40, at least 50, at least 60, at least 70, at least 80, at least 90, at least 100, at least 250, at least 500, at least 750, least 1000, at least 2000, at least 4000, at least 6000, at least 8000, at least 10000, at least 20000, at least 40000, at least 50000, at least 60000, at least 80000, or at least 100000 is reached. In certain embodiments, the amplification is terminated after an amplification factor of at most 2, at most 5, at most 10, at most 15, at most 20, at most 30, at most 40, at most 50, at most 60, at most 70, at most 80, at most 90, at most 100, at most 250, at most 500, at most 750, at most 1000, at most 2000, at most 4000, at most 6000, at most 8000, at most 10000, at most 20000, at most 40000, at most 50000, at most 60000, at most 80000, or at most 100000 is reached. As the use of MSRE or MSNE reduces or eliminates the production of hyper branching products, in certain embodiments wherein the MSRE or MSNE is used as disclosed in Section 5.2.4, the need to control the amplification factor is mitigated to a certain degree.

In certain embodiments, the amplification reaction is terminated after incubating with the DNA polymerase for a period of time. In certain embodiments, the amplification reaction is terminated after incubating with the DNA polymerase for at least 5 minutes, at least 15 minutes, at least 30 minutes, at least 1 hour, at least 2 hours, at least 3 hours, at least 4 hours, at least 5 hours, at least 6 hours, at least 8 hours, at least 10 hours, at least 12 hours, at least 18 hours, or at least 24 hours. In certain embodiments, the amplification reaction is terminated after incubating with the DNA polymerase for about 5 minutes, about 15 minutes, about 30 minutes, about 1 hour, about 2 hours, about 3 hours, about 4 hours, about 5 hours, about 6 hours, about 8 hours, about 10 hours, about 12 hours, about 18 hours, or about 24 hours.

In certain embodiments, the amplification action is actively terminated. In certain embodiments, actively terminating an amplification reaction comprises actively performing one or more steps to stop the amplification reaction. In certain embodiments, the amplification reaction can be actively terminated by heat inactivating the DNA polymerase of the RCA. One skilled in the art would appreciate that the inactivating temperature of the heat inactivation depends on the specific DNA polymerase used in the amplification reaction.

In certain embodiments, the amplification reaction is actively terminated by incubating the reaction mixture at an inactivating temperature of from about 65° C. to about 80° C., from about 70° C. to about 80° C., from about 75° C. to about 80° C., from about 65° C. to about 75° C., or from about 65° C. to about 70° C. In certain embodiments, the amplification reaction is actively terminated by increasing the incubation temperature to about 65° C., about 70° C., about 75° C., or about 80° C. In certain embodiments, the amplification reaction is actively terminated by increasing the incubation temperature to about 70° C.

In certain embodiments, the DNA polymerase (e.g., Deep Vent® DNA Polymerase) used herein cannot be heat inactivated. As such, the amplification reaction is terminated by digestion of the template and the amplification products.

In certain embodiments, the amplification reaction is actively terminated by incubating the reaction mixture at the inactivating temperature for from about 10 minutes to about 60 minutes. In certain embodiments, the amplification reaction is actively terminated by incubating the reaction mixture at the inactivating temperature for about 1 minute, about 5 minutes, about 10 minutes, about 15 minutes, about 20 minutes, about 25 minutes, about 30 minutes, about 45 minutes, or about 60 minutes. In certain embodiments, the amplification reaction is actively terminated by incubating the reaction mixture at the inactivating temperature for about 10 minutes.

In certain embodiments, the amplification reaction is actively terminated by incubating the reaction mixture at an inactivating temperature of about 55° C., about 60° C., about 65° C., about 70° C., about 75° C., about 80° C., about 85° C., about 90° C., about 95° C., or about 100° C. for about 1 minute, about 5 minutes, about 10 minutes, about 15 minutes, about 20 minutes, about 25 minutes, about 30 minutes, about 45 minutes, about 60 minutes, about 90 minutes, or about 120 minutes. In certain embodiments, the amplification reaction is actively terminated by incubating the reaction mixture at an inactivating temperature of from about 65° C. to about 80° C. for from about 10 minutes to about 60 minutes. In certain embodiments, the amplification reaction is actively terminated by incubating the reaction mixture at an inactivating temperature of about 70° C. for about 10 minutes.

In certain embodiments, the amplification reaction is actively terminated by adjusting the pH of the reaction mixture to a level outside the working pH range of the DNA polymerase. In certain embodiments, the amplification reaction is actively terminated by adjusting the pH of the amplification reaction mixture to at least 1, at least 2, at least 3 or more pH units higher than the highest working pH of the DNA polymerase. In certain embodiments, the amplification reaction is actively terminated by adjusting the pH of the amplification reaction mixture to about 10, about 11, about 12 or more. Non-limiting examples of buffers that can be used to increase the pH include phosphate buffers, carbonate buffers, NaOH, KOH, and NH₄OH. In certain embodiments, the amplification reaction is actively terminated by adjusting the pH of the amplification reaction mixture to at least 1, at least 2, at least 3 or more pH units lower than the lowest working pH of the DNA polymerase. In certain embodiments, the amplification reaction is actively terminated by adjusting the pH of the amplification reaction mixture to about 5, about 4, or about 3. Non-limiting examples of buffers that can be used to decrease the pH include citrate buffers, acetate buffers, and MES buffers.

In certain embodiments, the amplification reaction is actively terminated by removing one or more components in the amplification reaction required for DNA polymerase activity. In certain embodiments, the component required for DNA polymerase activity is a cation (e.g., a magnesium ion). In certain embodiments, the amplification reaction is actively terminated by adding one or more cationic chelating agents to the amplification reaction mixture to remove the cation. Non-limiting examples of cationic chelating agents that can be used with the present disclosure include nitrilotriacetic acid (NTA), ethylenediaminetetraacetic acid (EDTA), 1-(4,5-dimethoxy-2-nitrophenyl)-1,2-diaminoethane-N,N,N′,N′-tetraacetic acid (DMNP-EDTA), 1-hydroxy ethylidene-1,1-diphosphonic acid (HEDP), ethylenediamine tetra(methylene phosphonic acid) (EDTMPS), diethylenetriamine penta(methylene phosphonic acid) (DTPMPA), ethylenediamine-N,N′-bis(2-hydroxyphenylacetic acid) (EDDHA), sodium tripolyphosphate (STPP), sodium dextrose, and sodium metasilicate.

In certain embodiments, the amplification reaction is terminated by increasing the salt content of the reaction above the working range of the DNA polymerase. In certain embodiments, salts can be sodium chloride, potassium chloride, sodium acetate, potassium acetate, etc.

In certain embodiments, the amplification reaction is not actively terminated. In certain embodiments, the amplification reaction is terminated (i.e., additional amplification of the template DNA sequence is stopped) by proceeding to the next step(s) for the generation of hairpin-ended DNA molecules (see Section 5.3). In certain embodiments, where the next step toward generating hairpin-ended DNA molecules is creating single strand DNA overhangs using nicking endonuclease (see Section 5.3.2), the amplification reaction disclosed herein is not actively terminated before proceeding to the nicking step. In certain embodiments, adding the buffer for nicking endonuclease to the reaction mixture stops the amplification reaction. In certain embodiments, adding buffers for additional restriction enzymes can also terminate the reaction.

### 5.2.4 Digestion by MSRE or MSNE

MDA is an isothermal amplification method that primes and extends from a template to produce single-stranded DNA chains, which can be continuously re-primed and copies by strand-displacement synthesis. As DNA synthesis can be continuously primed and extended from many positions in the amplified molecules without required further rounds of denaturation, MDA can produce a network of hyper-branched DNA structures that has high viscosity. High viscosity of the amplification products creates a hydrogel of the reaction mixture that makes it difficult for the downstream restriction enzymes or nicking endonucleases to recognize and nick/cleave the amplification products (see Section 5.3.1 and Section 5.3.2). High viscosity and branching of the DNA products also reduces the fidelity of DNA polymerase for synthesis of the second strand of DNA resulting in the synthesis of double-stranded DNA. In addition, increased viscosity reduces the diffusion coefficient of components in the reaction, which slows down reaction rate and deceases enzyme activities. Adding MSRE or MSNE to the reaction mixture can reduce the viscosity and branching of the amplified DNA products, and thus improve the fidelity of the replication and increase the efficiency of the subsequence processes.

In certain embodiments, the DNA template and the amplification product produced therefrom comprise an MSRE site as disclosed in Section 5.1.3(a). As the MSRE site in the DNA template is methylated, and the MSRE site in the amplified amplification product is unmethylated, incubating the DNA template and the amplification product with an MSRE results in the cleavage of the amplification product at the unmethylated MSRE site while leaving the DNA template intact for further amplification. Such MSRE-mediated cleavage reduces the branching during the amplification as compared to the amplification without MSRE.

Alternatively, MSNE can be used in replace of the MSRE. In certain embodiments, the DNA template and the amplification product produced therefrom comprises MSNE sites as disclosed in Section 5.1.4(a). Similar to MSRE, as the MSNE site(s) in the DNA template is methylated, and the MSNE site(s) in the amplified amplification product is unmethylated, incubating the DNA template and the amplification product with an MSNE results in the cleavage of the amplification product at the unmethylated MSNE site while leaving the DNA template intact for further amplification.

As a result, the amplification products have reduced branching, reduced viscosity, and increased fidelity during the amplification as compared to the amplification performed in the absence of an MSRE or an MSNE. The location and number of the relevant restriction sites are described above in Sections 5.1.3(a) and 5.1.4(a).

One or more MSRE or MSNE can be added to the reaction mixture after the initiation of the amplification of the DNA template. In certain embodiments, MSRE or MSNE is added to the reaction mixture at the amplification reaction start or after the amplification factor reaches at least 1, at least 2, at least 5, at least 10, at least 15, at least 20, at least 30, at least 40, at least 50, at least 60, at least 70, at least 80, at least 90, or at least 100. In certain embodiments, one or more MSRE or MSNE is added to the reaction mixture 0 hours, 1 hour, 2 hours, 3 hours, 4 hours, 5 hours, 6 hours, 8 hours, 10 hours, 12 hours or more after the initiation of the amplification of the DNA template. In certain embodiments, MSRE or MSNE is added to the reaction mixture two or more times during the amplification reaction. In certain embodiments, digestion of the amplification products by MSRE or MSNE occur simultaneously with the amplification of the DNA template.

Exemplary MSRE that can be used with the presently disclosed subject matter include AlwNI, MscI, PflMI, SexAI, StuI, Acc65I, Asp718I, GsuI, SfoI, BsaBI, BspDI, BspEI, ClaI, NruI, AcIII, AccIII, and BseAI. Exemplary MSNE that can be used with the presently disclosed subject matter include Nt.BsaI and Nt.AlwI.

In certain embodiments, at least one MSRE and/or MSNE is added to the reaction mixture during the amplification reaction (i.e., before the termination of the amplification reaction) to prevent the reaction mixture from (i) becoming too viscous to allow proper mixing and/or (ii) showing decreased mass transfer, heat transfer and/or diffusion coefficients, which can impede the activities of the amplification enzymes and therefore undermine the fidelity and reaction rate of the amplification reaction. This is achieved by MSRE/MSNE of digesting de novo synthesized amplification products where the MSRE/MSNE sites are not methylated, while leaving the DNA template intact where the MSRE/MSNE sites are methylated. MSRE/MSNE digestion of de novo synthesized amplification products can reduce the propagation of errors.

Those skilled in the art understand that viscosity can be measured by various methods and devices like, for example, with glass capillary viscometers.

Concentrations of MSRE and/or MSNE in the reaction mixture can be determined by the skilled artisan based on the type of MSRE and/or MSNE used and the condition of the amplification. Ideally, MSRE or MSNE in the reaction mixture is at a suitable concentration that not all MSRE or MSNE sites are cleaved to ensure efficient DNA amplification.

In certain embodiments, the at least one MSRE and/or MSNE is added at a concentration (e.g., in mg/mL or molarity) or at an activity (e.g., in U/mL) having a specific ratio to the concentration of the DNA polymerase in the amplification reaction. In certain embodiments, the at least one MSRE and/or MSNE is added at an DNA polymerase:MSRE and/or MSNE ratio of about 1:1, about 1:0.8, about 1:0.6, about 1:0.4, about 1:0.2, about 1:0.05, about 1:0.01, about 1:0.005, about 1:0.001, about 1:0.0005, about 1:0.0001, about 1:0.00001, or less than 1:0.00001.

In certain embodiments, the at least one MSRE and/or MSNE is added to the reaction mixture at the initiation of the amplification. In certain embodiments, the at least one MSRE and/or MSNE is added to the reaction mixture after the initiation of the amplification. In certain embodiments, the at least one MSRE and/or MSNE is added to the reaction mixture about 0.5 hours, about 1.0 hour, about 2.0 hours, about 3.0 hours, about 4.0 hours, about 6.0 hours, about 10.0 hours, about 15.0 hours, about 20.0 hours, or about 24 hours after the initiation of the amplification reaction by the DNA polymerase.

In certain embodiments, the at least one MSRE and/or MSNE is added to the reaction mixture at an amount having a specific ratio to the amount of DNA template in the reaction mixture at the initiation of the amplification reaction. In certain embodiments, the at least one MSRE and/or MSNE is added to the reaction mixture at a DNA template:MSRE and/or MSNE weight to weight ratio of about 1:1, about 1:0.8, about 1:0.6, about 1:0.4, about 1:0.2, about 1:0.05, about 1:0.01, about 1:0.005, about 1:0.001, about 1:0.0005, about 1:0.0001, about 1:0.00001, or less than about 1:0.00001. In certain embodiments, the at least one MSRE and/or MSNE is added to the reaction mixture at a molar ratio of DNA template:MSRE and/or MSNE, where every copy, every second, every third, every fourth, every sixth, every tenth, every hundred, or every thousand copies of the nascent amplification product copied from the DNA template is digested by the MSRE and/or MSNE.

In certain embodiments, the at least one MSRE and/or MSNE is added to the reaction mixture when the viscosity measurement of the reaction mixture reaches a certain level, which is caused by the hyper-branching of the amplified product. In certain embodiments, at least one MSRE and/or MSNE is added to the reaction mixture when the reaction mixture has a viscosity of at most 5 mPa·s, at most 10 mPa's, at most 50 mPa·s, at most 100 mPa·s, at most 200 mPa·s, at most 400 mPa·s, at most 500 mPa·s, at most 1000 mPa·s, at most 2500 mPa·s, at most 5000 mPa's, or at most 10,000 mPa·s. In certain embodiments, at least one MSRE and/or MSNE is added to the reaction mixture to control the viscosity of the reaction mixture to be below a critical viscosity value.

In certain embodiments, the amplified amplification product has reduced branching as compared to the amplification performed in the absence of an MSRE or an MSNE. In certain embodiments, the branching of the amplified amplification products is reduced by the MSRE or the MSNE to at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, or at most about 5% of the branching of the amplified amplification products in the absence of an MSRE or an MSNE. In certain embodiments, the branching of the amplified amplification products is reduced by the MSRE or the MSNE to about 80%, about 75%, about 70%, about 65%, about 60%, about 55%, about 50%, about 45%, about 40%, about 35%, about 30%, about 25%, about 20%, about 15%, about 10%, or about 5% of the branching of the amplified amplification products in the absence of an MSRE or an MSNE.

In certain embodiments, the amplified amplification product has reduced viscosity as compared to amplification performed in the absence of an MSRE or an MSNE. In certain embodiments, the viscosity of the amplified amplification products is reduced by the MSRE or the MSNE to at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, or at most about 5% of the viscosity of the amplified amplification products in the absence of an MSRE or an MSNE. In certain embodiments, the viscosity of the amplified amplification products is reduced by the MSRE or the MSNE to about 80%, about 75%, about 70%, about 65%, about 60%, about 55%, about 50%, about 45%, about 40%, about 35%, about 30%, about 25%, about 20%, about 15%, about 10%, or about 5% of the viscosity of the amplified amplification products in the absence of an MSRE or an MSNE.

In certain embodiments, the viscosity of the amplified amplification products digested with the MSRE or MSNE is at most 5 mPa·s, at most 10 mPa·s, at most 50 mPa·s, at most 100 mPa·s, at most 200 mPa·s, at most 400 mPa·s, at most 500 mPa·s, at most 1000 mPa·s, at most 2500 mPa·s, at most 5000 mPa·s, or at most 10,000 mPa·s.

### 5.2.5 Additional Steps

Additional steps can be performed after the conclusion of the amplification (see Sections 5.2.1-5.2.4) and prior to the initiation of making hairpin-ended DNA molecules (see Section 5.3). In certain embodiments, an additional step is performed to prepare the reaction mixture and optimize the reaction conditions for the subsequent step of making hairpin-ended DNA molecules (e.g., the step of making precursors of hairpin-ended DNA molecules as described in Section 5.3.1, or the step of incubating with nicking endonucleases to create single strand DNA overhangs as described in Section 5.3.2).

In certain embodiments, the additional steps comprise buffer exchange, which replaces the buffer for amplification with a buffer for the enzymes used in the next step (e.g., the step of making precursors of hairpin-ended DNA molecules as described in Section 5.3.1, or the step of incubating with nicking endonucleases to create single strand DNA overhangs as described in Section 5.3.2). In certain embodiments, the additional steps comprise concentrating the amplification product. In certain embodiments, the additional steps comprise removing the DNA template, DNA polymerase and/or primers from the reaction mixture. In certain embodiments, the additional steps are performed using tangential flow filtration (TFF), attenuated tangential flow-filtration (ATF), or TFDFR (Tangential Flow Depth Filtration). In certain embodiments, an additional step is added for industrial scale production of the hairpin-ended DNA molecules. In certain embodiments, the amplification reaction is a batch process or a continuous flow process. In certain embodiments, further components (e.g., enzymes, polymerase, primers, dNTPs or buffers) can be supplied to the reaction as required during the process. Should further additions be required, it will dilute the concentration of the nucleotide or nucleotide complexes, unless further nucleotide complexes are added to replenish or increase the concentration. In certain embodiments, a continuous flow process can be used to adapt or change reaction components based on the progression of the reaction.

In certain embodiments, both steps of the amplification of the DNA template and making hairpin-ended DNA molecules are carried out in the same container or reaction mixture (e.g., one pot reaction). In certain embodiments, a buffer or a reagent(s) is added to the reaction mixture at the end of the amplification to provide conditions for the nicking endonuclease to act.

In certain embodiments, the reactions are carried out continuously, where no additional steps (e.g., buffer exchange, removing molecules from the reaction mixture, concentrating DNA molecules) are included between the conclusion of the amplification of the DNA template and the initiation of making hairpin-ended DNA molecules. In certain embodiments, the reactions are carried out following an additional step to remove certain components or impurities of the reaction mixture by common techniques known in the art (e.g., buffer exchange, selective removal of components by chromatographic methods).

### 5.3 Methods of Making Hairpin-ended DNA Molecules

Amplification products amplified from the DNA template disclosed herein are further processed to produce hairpin-ended DNA molecules of interest (i.e., hairpin-ended DNA molecules comprising a sequence of interest).

### 5.3.1 Making Precursors of Hairpin-ended DNA Molecules

In certain embodiments, the amplification products are first processed to generate precursors of hairpin-ended DNA molecules before being further processed to generate hairpin-ended DNA molecules.

In certain embodiments, the amplification product is incubated with a restriction enzyme, where the restriction enzyme cleaves at the restriction enzyme site as disclosed in Section 5.1.3. In certain embodiments, the amplification product is incubated with an MSRE, wherein the MSRE cleaves at the MSRE site as disclosed in Section 5.1.3(a). In certain embodiments, the amplification product is incubated with at least one nicking endonuclease, where the nicking endonuclease nicks at the nicking endonuclease sites as disclosed in Section 5.1.4. In certain embodiments, the amplification product is incubated with an MSNE, where the MSNE nicks at the MSNE sites as disclosed in Section 5.1.4(a). Such cleaving or nicking creates double-strand breaks in the amplification product, and breaks the amplification product into fragments, where some of the fragments comprise the sequence of interest (i.e., precursor of hairpin-ended DNA molecule). The precursor of hairpin-ended DNA molecule is further processed according to Sections 5.3.2-5.3.4 to produce the hairpin-ended DNA molecule of interest.

Without being bound by the theory, a nicking endonuclease recognizes the restriction sites for the nicking endonuclease (i.e., nicking endonuclease sites) in the DNA molecule and cuts only on one strand (e.g., hydrolyzes the phosphodiester bond of a single DNA strand) of the dsDNA at a site that is either within or outside the restriction sites for the nicking endonuclease, thereby creating a nick in the dsDNA. A restriction enzyme, on the other hand, recognizes the restriction sites for the restriction enzyme and cuts both strands of the dsDNA, thereby cleaving DNA molecules at or near the specific restriction sites.

Exemplary restriction enzymes and nicking endonuclease and reaction conditions are disclosed in Sections 5.1.3, 5.1.4, 5.3.2, and 5.4.1.

Exemplary MSREs and reaction conditions that can be used with the present disclosure are disclosed in Sections 5.1.3(a) and 5.2.4.

Exemplary MSNEs and reaction conditions that can be used with the present disclosure are disclosed in Sections 5.1.4(a) and 5.2.4.

### 5.3.2 Creating Single Strand DNA Overhangs Using Nicking Endonucleases

In certain embodiments, the amplification product or the precursor of hairpin-ended DNA molecule is incubated with one or more nicking endonucleases, which nick at the nicking endonuclease sites (e.g., nicking endonuclease sites disclosed in Section 5.1.1(b)) to create the DNA molecule comprising single strand DNA overhangs, which are further processed to form the hairpin-ended DNA molecule.

Any suitable nicking endonucleases known and practiced in the art can be used with the presently disclosed subject matter. In certain embodiments, the nicking endonucleases are naturally occurring nicking endonucleases that are not 5-methylcytosine dependent, including Nb.BsmI, Nb.BbvCI, Nb.BsrDI, Nb.BtsI, Nt.BbvCI, Nt.AlwI, Nt. CviPII, Nt. BsmAI, Nt. AlwI, and Nt.BstNBI. Nicking endonucleases used herein can also be engineered from Type IIs restriction enzymes (e.g., AlwI, BpulOI, BbvCI, BsaI, BsmBI, BsmAI, BsmI, BspOJ, MlyI, Mval2691 and SapI, etc.). Methods of making nicking endonucleases can be found, for example in, U.S. Pat. Nos. 7,081,358; 7,011,966; 7,943,303; 7,820,424; and WO201804514, each of which is herein incorporated in its entirety by reference.

Alternatively, a programmable nicking enzyme can be used with the presently disclosed subject matter in place of the nicking endonuclease. Exemplary programmable nicking enzymes that can be used with the presently disclosed subject matter include, Cas9 or a functional equivalent thereof (such as Pyrococcus furiosus Argonaute (PfAgo) or Cpf1). Cas9 contains two catalytic domains, RuvC and HNH, where inactivating one of RuvC and HNH generates a programmable nicking enzyme. The RuvC domain can be inactivated by an amino acid substitution at position D10 (e.g., D10A), and the HNH domain can be inactivated by an amino acid substitution at position H840 (e.g., H840A), or at a position corresponding to those amino acids in other Cas9 equivalent proteins. The programmable nicking enzyme can also be Argonaute or Type II CRISPR/Cas endonucleases that comprise two components: 1) a nicking enzyme (e.g., a D10A Cas9 nicking enzyme or variant or ortholog thereof) that cleaves the target DNA; and 2) a guide nucleic acid, e.g., a guide DNA or RNA (gDNA or gRNA) that targets or programs the nicking enzyme to a specific site in the target DNA (see, e.g., Hsu, et al., Nature Biotechnology 2013 31:827-832, which is herein incorporated in its entirety by reference). A programmable nicking enzyme can also be made by fusing a site specific DNA binding domain (targeting domain) such as the DNA binding domain of a DNA binding protein (e.g., a restriction endonuclease, a transcription factor, a zinc-finger or another domain in that binds to DNA at non-random positions) with a nicking endonuclease so that it acts on a specific, non-random site. The programmable cleavage by a programmable nicking enzyme results from targeting domain within or fused to the nicking enzyme or from guide molecules (gDNA or gRNA) that direct the nicking enzyme to a specific, non-random site, which can be programmed by changing the targeting domain or the guide molecule. Exemplary programmable nicking enzymes that can be used with the present disclosure can be found, for example, in U.S. Pat. No. 7,081,358 and WO2010021692A, which are herein incorporated in their entireties by reference.

Suitable guide nucleic acid (e.g., gDNA or gRNA) sequences and suitable target sites for the guide nucleic acid have been known and widely utilized in the art. The guide nucleic acid (e.g., gDNA or gRNA) is a specific nucleic acid (e.g., gDNA or gRNA) sequence that recognizes the target DNA region of interest and directs the programmable nicking enzyme (e.g., Cas nuclease) there for editing. The guide nucleic acid (e.g., gDNA or gRNA) often comprises two parts: a targeting nucleic acid, a 15-20 nucleotide sequence complementary to the target DNA, and a scaffold nucleic acid, which serves as a binding scaffold for the programmable nicking enzyme (e.g., Cas nuclease). Suitable target sites for the guide nucleic acid comprise two components: a complementary sequence to the targeting nucleic acid in the programmable nicking enzyme and an adjacent Protospacer Adjacent Motif (PAM). The PAM serves as a binding signal for the programmable nicking enzyme (e.g., Cas nuclease). Various PAMs have been known, characterized, and utilized in the art, for example as discussed in Daniel Gleditzsch et al., RNA Biol. 16(4): 504-517 (April 2019); Ryan T. Leenay et al., Mol Cell. 62(1): 137-147 (Apr. 7, 2016), both of which are herein incorporated in their entirety by reference. Exemplary gRNA and gDNA sequences targeting the primary stem sequence of AAV2 ITRs include such listed in Table 20.

TABLE 20
Exemplary gRNA and gDNA Sequences

| | |
|---|---|
| SEQ ID NO: 493 | AGCGAGCGAGCG |
| AAV2 wt gRNA for | CGCAGAGAGGG |
| Nicking Cas9 | |
| SEQ ID NO: 494 | GCTCGCTCGCTC |
| AAV2 wt gDNA for | GGTG |
| PfAgo | |

In certain embodiments, the first, second, third, and fourth restriction sites are targeted and nicked by the same nicking endonuclease. In certain embodiments, the first, second, third, and fourth restriction sites for nicking endonuclease are targeted and nicked by two or more different nicking endonucleases, e.g., two, three or four different nicking endonucleases. In certain embodiments, each of the two or more different nicking endonucleases target one or more different restriction sites of the first, second, third, and fourth restriction sites.

Any suitable nicking endonucleases known in the art can be used with the present disclosure. Exemplary nicking endonucleases and corresponding restriction sites that can be used with the present disclosure are described in Table 21 and The Restriction Enzyme Database (known in the art as REBASE), which is available at www.rebase.neb.com/cgi-bin/azlist?nick and incorporated herein in its entirety by reference.

TABLE 21
Exemplary Nicking Endonuclease and Their
Corresponding Restriction Sites

| | | Corresponding Restriction |
|---|---|---|
| | | Sites for the Nicking |
| | | Endonuclease and |
| | | Position of Nick Relative |
| | | to the Restriction Sites |
| | | (Note: |
| | | 1/none means the nick |
| | | is 1 nucleotide |
| | SEQ | 3′ from the |
| Nicking | ID | restriction sites |
| Endonuclease | NO | on the top strand) |
| Nt. BsmAI | 495 | GTCTC (1/none) |
| Nt. BtsCI | 496 | GGATG (2/none) |
| N. ALwl | 497 | GGATC (4/none) |
| N. BstNBI | 498 | GAGTC (4/none) |
| N. BspD6I | 499 | GAGTC (4/none) |
| Nb. Mva1269I | 500 | GAATGC (none/−1) |
| Nb. BsrDI | 501 | GCAATG (none/0) |
| Nb. BtsI | 502 | GCAGTG (none/0) |
| Nt. BtsI | 503 | GCAGTG (2/none) |
| Nt. Bsal | 504 | GGTCTC (1/none) |
| Nt. Bpu10I | 505 | CCTNAGC (−5/none) |
| Nb.Bpu10I | 506 | CCTNAGC (none/−2) |
| Nt. BsmBI | 507 | CGTCTC (1/none) |
| Nb. BbvCI | 508 | CCTCAGC (none/−2) |
| Nt. BbvCI | 509 | CCTCAGC (−5/none) |
| Nt. BspQI | 510 | GCTCTTC (1/none) |

Conditions for nicking a DNA strand by nicking endonuclease(s) are known in the art, including temperatures, salt concentrations, pH, buffering reagents, the presence or absence of certain detergent, and duration of incubation to achieve the desired percentage of nicked DNA molecules. These conditions are readily available from the websites or catalogs of various vendors of the nicking endonucleases, e.g., New England BioLabs (NEB). Methods disclosed herein comprise incubating the amplification product or precursor of hairpin-ended DNA molecule with one or more nicking endonucleases under conditions known and practiced in the art, or optimized by methods known in the art.

In certain embodiments, conditions for nicking a DNA strand by nicking endonuclease can be determined by one skilled in the art using know methods (e.g., checking the nicking products on an agarose gel after the incubation with the nicking endonuclease).

### 5.3.3 Denaturing Nicked DNA Molecules to Create Single-Strand Overhangs

In certain embodiments, the methods disclosed herein further comprises denaturing the nicked DNA molecule to create a DNA fragment that comprises two single strand DNA overhangs as disclosed in Sections 5.1.1(a), 5.3.3, and 5.1.1(b) upon separation of the top from the bottom strand.

Denaturing of a DNA molecules can be done by various ways as known and practiced in the art. In certain embodiments, the denaturing separates a double strand DNA (dsDNA) into two single strand DNA (ssDNA). In certain embodiments, the denaturing comprises increasing the temperature until the DNA unwinds and the hydrogen bonds that hold the two strands together weaken and finally break. The process of separating double-stranded DNA into single strands is known as DNA denaturation, or DNA denaturing.

In certain embodiments, denaturing the DNA molecule comprises denaturing one or more segments of the dsDNA molecule, while keeping the other segment(s) of the DNA molecule as dsDNA. In certain embodiments, denaturing the DNA molecule comprises denaturing the whole segment of the dsDNA molecule into ssDNA strands.

In certain embodiments, denaturing the DNA molecule can separate the dsDNA molecule into ssDNA at the segment between the first and second restriction sites for nicking endonuclease on the top and bottom strands of the amplification product or precursor of hairpin-ended DNA molecule while keeping the other segments of the DNA molecule as dsDNA, thereby creating an overhang between the first and second restriction sites. In certain embodiments, denaturing the DNA molecule can separate the dsDNA molecule into ssDNA at the segment between the third and fourth restriction sites for nicking endonuclease on the top and bottom strands of the amplification product or precursor of hairpin-ended DNA molecule while keeping the other segments of the DNA molecule as dsDNA, thereby creating an overhang between the third and fourth restriction sites. In certain embodiments, denaturing the DNA molecules can separate the dsDNA into ssDNA at the segments between the first and second restriction sites and between the third and fourth restriction sites for nicking endonuclease on the top and bottom strand of the amplification product or precursor of hairpin-ended DNA molecule, while keeping the other segments of the DNA molecule as dsDNA, thereby (1) breaking the DNA molecule into two daughter DNA molecules and (2) creating an overhang between the first and second restriction sites and an overhang between the third and fourth restriction sites. In certain embodiments, the overhang between the first and second restriction sites for nicking endonuclease can be a top strand 5′ overhang. In certain embodiments, the overhang between the first and second restriction sites for nicking endonuclease can be a bottom strand 3′ overhang. In certain embodiments, the overhang between the third and fourth restriction sites for nicking endonuclease can be a top strand 3′ overhang. In certain embodiments, the overhang between the third and fourth restriction sites for nicking endonuclease can be a bottom strand 5′ overhang. In certain embodiments, the step of denaturing the DNA molecule can separate the DNA molecules in any combinations of the embodiments provided herein.

The overhangs can vary in length depending on the distance between the restriction sites for nicking endonuclease. In certain embodiments, the overhangs are identical in length and/or sequences. In certain embodiments, the overhangs are different in length and/or sequences. In certain embodiments, a top strand 5′ overhang is at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 nucleotides in length. In certain embodiments, a top strand 5′ overhang is about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, or more nucleotides in length. In certain embodiments, a bottom strand 3′ overhang is at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 nucleotides in length. In certain embodiments, a bottom strand 3′ overhang is about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, or more nucleotides in length. In certain embodiments, a top strand 3′ overhang is at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 nucleotides in length. In certain embodiments, a top strand 3′ overhang is about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, or more nucleotides in length. In certain embodiments, a bottom strand 5′ overhang is at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 nucleotides in length. In certain embodiments, a bottom strand 5′ overhang is about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, or more nucleotides in length.

Any suitable methods known in the art can be used for denaturing DNA molecules of the present disclosure, such as denaturing by heat, by changing the pH in the environment of the DNA molecules, by increasing the salt concentration, or by any combination of these and other known means. The present disclosure provides that the DNA molecules can be denatured under conditions that selectively separates the dsDNA into ssDNA at the segments between the first and second restriction sites and/or between the third and fourth restriction sites on the top and bottom strand of the DNA, while keeping the other part of the DNA molecule as dsDNA. Such selective separating of dsDNA to ssDNA can be performed by controlling the denaturing conditions and/or the time the DNA molecules are subjected to the denaturing conditions. In certain embodiments, the DNA molecules are denatured at a temperature of at least 70° C., at least 71° C., at least 72° C., at least 73° C., at least 74° C., at least 75° C., at least 76° C., at least 77° C., at least 78° C., at least 79° C., at least 80° C., at least 81° C., at least 82° C., at least 83° C., at least 84° C., at least 85° C., at least 86° C., at least 87° C., at least 88° C., at least 89° C., at least 90° C., at least 91° C., at least 92° C., at least 93° C., at least 94° C., or at least 95° C. In certain embodiments, the DNA molecules are denatured at a temperature of about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., or about 95° C. In certain embodiments, the DNA molecules are denatured at a temperature of about 90° C.

Segments of or the whole DNA molecule provided herein can undergo the denaturation process by addition of chemical agents, such as guanidine, formamide, sodium salicylate, dimethyl sulfoxide, propylene glycol, and urea. These chemical denaturing agents can lower the melting temperature by competing for hydrogen bond donors and acceptors with pre-existing nitrogenous base pairs and allow for isothermal denaturing. In certain embodiments, chemical agents are able to induce denaturation at room temperature. In certain embodiments, alkaline agents (e.g., NaOH) can be used to denature DNA by changing pH and removing hydrogen-bond contributing protons. In certain embodiments, chemically denaturing the DNA molecules provided herein can be a gentler procedure for DNA stability compared to denaturation induced by heat. In certain embodiments, chemically denaturing and renaturing the DNA molecules (e.g., changing the pH) provided herein can be a quicker than by heating. In certain embodiments, the DNA of the present disclosure can be replicated and nicked in bacteria and denatured simultaneously during the release (e.g., alkali lysis step) from bacteria.

In certain embodiments, the DNA molecules are denatured at a pH of at least 10, at least 10.1, at least 10.2, at least 10.3, at least 10.4, at least 10.5, at least 10.6, at least 10.7, at least 10.8, at least 10.9, at least 11, at least 11.1, at least 11.2, at least 11.3, at least 11.4, at least 11.5, at least 11.6, at least 11.7, at least 11.8, at least 11.9, at least 12, at least 12.1, at least 12.2, at least 12.3, at least 12.4, at least 12.5, at least 13, at least 13.5, or at least 14. In certain embodiments, the DNA molecules are denatured at a pH of about 10, about 10.1, about 10.2, about 10.3, about 10.4, about 10.5, about 10.6, about 10.7, about 10.8, about 10.9, about 11, about 11.1, about 11.2, about 11.3, about 11.4, about 11.5, about 11.6, about 11.7, about 11.8, about 11.9, about 12, about 12.1, about 12.2, about 12.3, about 12.4, about 12.5, about 13, about 13.5, or about 14. In certain embodiments, the DNA molecules are denatured at a salt concentration of at least 1 M, at least 1.5 M, at least 2 M, at least 2.5 M, at least 3 M, at least 3.5 M, or at least 4 M of salt. In certain embodiments, the DNA molecules are denatured at a salt concentration of about 1 M, about 1.5 M, about 2 M, about 2.5 M, about 3 M, about 3.5 M, or about 4 M of salt. In certain embodiments, the DNA molecule is subject to the denaturing condition for at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 minutes. In certain embodiments, the DNA molecule is subject to the denaturing condition for about 1, about 2, about 3, about 4, about 5, about 6, about 7, about 8, about 9, about 10, about 11, about 12, about 13, about 14, about 15, about 16, about 17, about 18, about 19, or about 20 minutes. In certain embodiments, the DNA molecules can be denatured by any combination of denaturing conditions and duration of denaturing as provided herein.

Selectively denaturing some segments the DNA molecule can be achieved by selective denaturing conditions that are determined according to the properties of the DNA segments to be selectively denatured. The stability of the DNA double helix correlates with the length of the DNA segments and the percentage of G/C content. The present disclosure provides that the selective denaturing conditions can be determined by the sequence of the DNA segments to be selectively denatured or the resulting sequence of the overhang. For example, the temperature for selective denaturing can be approximately determined as Tm=2° C.×number of A-T pair+4° C.×number of G-C pair for a DNA sequence to be selectively denatured. Other more precise calculations of the Tm are also known and used in the art, for example, as described in Freier S M, et a., Proc Natl Acad Sci, 83, 9373-9377 (1986); Breslauer K J, et al., Proc Natl Acad Sci, 83, 3746-3750 (1986); Panjkovich, A. and Melo, F. Bioinformatics 21:711-722 (2005); Panjkovich, A., et al. Nucleic Acids Res 33:W570-W572 (2005), all of which are herein incorporated in their entireties by reference.

The overhang can comprise various DNA sequences. In certain embodiments, the overhang comprises an inverted repeat or a fragment thereof (e.g., at least 50%, 60%, 70%, 75%, 80%, 85%, 90%, 95%, or 99% of an inverted repeat). In certain embodiments, the overhang comprises a viral inverted repeat or a fragment thereof (e.g., at least 50%, 60%, 70%, 75%, 80%, 85%, 90%, 95%, or 99% of a viral inverted repeat). In certain embodiments, the overhang comprises or consists of any embodiments of sequences described in Section 5.1.1.

### 5.3.4 Annealing to Form Hairpin-ended DNA Molecules

Methods of making hairpin-ended DNA molecules disclosed herein further comprise selectively annealing intramolecularly the ssDNA overhangs resulting from the denaturing as disclosed in Section 5.3.3. Such annealing creates a hairpinned inverted repeat on each end of the DNA molecule to form the hairpin-ended DNA molecules disclosed herein (e.g., the hairpin-ended DNA molecules of Section 5.1.1).

The present disclosure provides that the ssDNA overhang can be designed to have certain lengths, numbers of complementary nucleotide pairs, and percentage of G-C and A-T pairs, such that the free energy gain (ΔG=ΔH−TΔS) of intramolecular annealing of the overhang is bigger over that of intermolecular annealing, thereby making the intramolecular annealing thermodynamically favored over the intermolecular annealing. As the nucleotides within the ssDNA overhang have a higher probability of contacting each other than contacting the nucleotides of another ssDNA overhang in molecular motion, the kinetics of intramolecular annealing of the ssDNA overhang can be higher than that of intermolecular annealing. Therefore, even if the intramolecular annealing is thermodynamically disfavored over the intermolecular annealing, the superior kinetics of intramolecular annealing of the ssDNA overhang can result in the formation of intramolecularly annealed overhang over intermolecularly annealed overhang.

Suitable annealing temperatures can be adopted to favor the intramolecular annealing over intermolecular annealing. In certain embodiments, the ssDNA overhang is annealed at a temperature of at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., at least 30° C., at least 31° C., at least 32° C., at least 33° C., at least 34° C., at least 35° C., at least 36° C., at least 37° C., at least 38° C., at least 39° C., at least 40° C., at least 41° C., at least 42° C., at least 43° C., at least 44° C., at least 45° C., at least 46° C., at least 47° C., at least 48° C., at least 49° C., at least 50° C., at least 51° C., at least 52° C., at least 53° C., at least 54° C., at least 55° C., at least 56° C., at least 57° C., at least 58° C., at least 59° C., or at least 60° C. In certain embodiments, the ssDNA overhang is annealed at a temperature of about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., or about 60° C. In certain embodiments, the ssDNA overhang is annealed at a temperature of at least 25° C. In certain embodiments, the ssDNA overhang is annealed at a temperature of about 25° C. In certain embodiments, the ssDNA overhang is annealed at room temperature.

Suitable annealing durations can be adopted to favor the intramolecular annealing over intermolecular annealing. In certain embodiments, the ssDNA overhang is annealed for at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40 minutes. In certain embodiments, the ssDNA overhang is annealed for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 minutes. In certain embodiments, the ssDNA overhang is annealed for at least 20 minutes. In certain embodiments, the ssDNA overhang is annealed for about 20 minutes.

In certain embodiments, annealing can be achieved by lowering the temperature below the calculated melting temperatures of the sense and antisense sequence pairs. The melting temperature is dependent upon the specific nucleotide base content and the characteristics of the solution being used, e.g., the salt concentration. Melting temperatures for any given sequence and solution combination are readily calculated as known and practiced in the art.

In certain embodiments, annealing can be accomplished isothermally by reducing the amount of denaturing chemical agents to allow an interaction between the sense and antisense sequence pairs. The minimum concentration of denaturing chemical agents required to denature the DNA sequence can be dependent upon the specific nucleotide base content and the characteristics of the solution being used, e.g., temperature or the salt concentration. The concentration of chemical denaturing agents that do not lead to denaturing for any given sequence and solution combination are readily identified as known and practiced in the art. The concentration of chemical denaturing agents can also be readily modified as known and practiced in the art. For example, the amount of urea can be lowered by dialysis or tangential flow filtration or the pH can be changed by the addition of acids or bases.

The annealing temperature and the annealing duration for intramolecular annealing depend on lengths, the number of complementary nucleotide pairs, and percentage of G-C and A-T pairs of the ssDNA overhang, and the sequence of the ssDNA overhang (the arrangement of the complementary nucleotide pairs). In certain embodiments, an ssDNA overhang provided for the methods provided herein comprises any number of nucleotides in length as described in Section 5.3.3. In certain embodiments, a ssDNA overhang provided for the methods provided herein comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 intramolecularly complementary nucleotide pairs. In certain embodiments, a ssDNA overhang provided for the methods provided herein comprises about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 intramolecularly complementary nucleotide pairs. In certain embodiments, a ssDNA overhang provided for the methods provided herein comprises at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, or at least 90% G-C pairs among intramolecularly complementary nucleotide pairs. In certain embodiments, a ssDNA overhang provided for the methods provided herein comprises about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% G-C pairs among intramolecularly complementary nucleotide pairs.

Additionally, the concentration of the DNA molecules, which correlates with the concentration of the overhangs, can affect the equilibrium and kinetics of the intramolecular annealing and the intermolecular annealing of the overhangs. Without being bound or otherwise limited by the theory, when the concentration of the overhang is too high, the probability of the intermolecular contact among the overhangs increases and the kinetic advantage of the intramolecular contact over intermolecular contact seen at lower concentration as discussed above is then diminished.

In certain embodiments, intramolecular interactions can occur at a faster rate while intermolecular interactions occur at a slower rate. In certain embodiments, base pair interactions involving three or more molecules (e.g., three different strands) occur at the slowest rate. In certain embodiments, the kinetic rate of intramolecular interactions versus intermolecular interactions is governed by the concentration of each molecule. In certain embodiments, the intramolecular interactions are kinetically faster or intramolecular forces are larger when the concentration of DNA strands is lower.

Viewed individually, the absolute free energy of forming each complementary domain of IRs or ITRs, may be different, leading to regions of the IR or ITR that may locally fold earlier as the strand transitions from a denatured to annealed state. The presence of locally folded domains (e.g., a central hairpin or branched hairpin like in AAV2 ITRs as described in Sections 5.1.1 and 5.3) can reduce the amount of bases available for pairing with other strands and thus can reduce the likelihood of intermolecular annealing or hybridization and shift the equilibrium from intermolecular annealing to intramolecular annealing or ITR formation.

Accordingly, the present disclosure provides that the annealing step can be performed at various concentrations to favor the intramolecular annealing over intermolecular annealing. In certain embodiments, the ssDNA overhang is annealed at a concentration of at least 1 ng/μl, at least 5 ng/μl, at least 10 ng/μl, at least 20 ng/μl, at least 30 ng/μl, at least 40 ng/μl, at least 50 ng/μl, at least 60 ng/μl, at least 80 ng/μl, at least 85 ng/μl, at least 100 ng/μl, at least 150 ng/μl, at least 200 ng/μl, at least 300 ng/μl, at least 400 ng/μl, at least 500 ng/μl, at least 600 ng/μl, at least 800 ng/μl, at least 1000 ng/μl, at least 2000 ng/μl, at least 3000 ng/μl, at least 4000 ng/μl, at least 5000 ng/μl, at least 6000 ng/μl, at least 7000 ng/μl, at least 8000 ng/μl, at least 9000 ng/μl, at least 10000 ng/μl or more for the DNA molecules. In certain embodiments, the ssDNA overhang is annealed at a concentration of about 1 ng/μl, about 5 ng/μl, about 10 ng/μl, about 20 ng/μl, about 30 ng/μl, about 40 ng/μl, about 50 ng/μl, about 60 ng/μl, about 80 ng/μl, about 85 ng/μl, about 100 ng/μl, about 150 ng/μl, about 200 ng/μl, about 300 ng/μl, about 400 ng/μl, about 500 ng/μl, about 600 ng/μl, about 800 ng/μl, about 1000 ng/μl, about 2000 ng/μl, about 3000 ng/μl, about 4000 ng/μl, about 5000 ng/μl, about 6000 ng/μl, about 7000 ng/μl, about 8000 ng/μl, about 9000 ng/μl, about 10000 ng/μl or more for the DNA molecules.

Similarly, the present disclosure provides that the annealing step can be performed at various molar concentrations to favor the intramolecular annealing over intermolecular annealing. In certain embodiments, the ssDNA overhang is annealed at a concentration of at least 1 nM, at least 5 nM, at least 10 nM, at least 20 nM, at least 30 nM, at least 40 nM, at least 50 nM, at least 60 nM, at least 80 nM, at least 85 nM, at least 100 nM, at least 150 nM, at least 200 nM, at least 300 nM, at least 400 nM, at least 500 nM, at least 600 nM, at least 800 nM, at least 1000 nM, at least 2000 nM, at least 3000 nM, at least 4000 nM, at least 5000 nM, at least 6000 nM, at least 7000 nM, at least 8000 nM, at least 9000 nM, at least 10000 nM or more for the DNA molecules. In certain embodiments, the ssDNA overhang is annealed at a concentration of about 1 nM, about 5 nM, about 10 nM, about 20 nM, about 30 nM, about 40 nM, about 50 nM, about 60 nM, about 80 nM, about 85 nM, about 100 nM, about 150 nM, about 200 nM, about 300 nM, about 400 nM, about 500 nM, about 600 nM, about 800 nM, about 1000 nM, about 2000 nM, about 3000 nM, about 4000 nM, about 5000 nM, about 6000 nM, about 7000 nM, about 8000 nM, about 9000 nM, about 10000 nM or more for the DNA molecules. In certain embodiments, the ssDNA overhang is annealed at a concentration of at about 3000 ng/μl. In certain embodiments, the ssDNA overhang is annealed at a concentration of at about 3000 nM.

In certain embodiments, an ssDNA overhang provided for the methods provided herein comprises any one of SEQ ID NOs: 1-15.

In certain embodiments, the structure of the DNA molecules provided herein is the same after 2, 3, 4, 5, 10 or 20 cycles of denaturing/renaturing (e.g., denaturing as described in Section 5.3.3 and re-annealing as described in Section 5.3.4). DNA structures can be described by an ensemble of structures at or around the energy minimum. In certain embodiments, the ensemble DNA structure is the same after 2, 3, 4, 5, 10 or 20 cycles of denaturing/renaturing. In certain embodiments, the folded hairpin structure formed from the ITR or IR provided herein is the same after 2, 3, 4, 5, 10 or 20 cycles of denaturing/renaturing. In certain embodiments, the ensemble structure of the folded hairpin is the same after 2, 3, 4, 5, 10 or 20 cycles of denaturing/renaturing.

5.3.5 Repairing the Nicks with a Ligase

Optionally, methods disclosed herein further comprise repairing the nicks with a ligase after the annealing as described in Section 5.3.4.

DNA ligases catalyze the joining of two ends of DNA molecules by forming one or more new covalent bonds. For example, commonly used T4 DNA ligase catalyzes the formation of a phosphodiester bond between juxtaposed 5′ phosphate and 3′ hydroxyl termini in DNA. The formation of new covalent bonds that are catalyzed by ligase to join two DNA molecules is referred to as “ligation.” In certain embodiments, methods disclosed herein further comprise ligating nucleotides with no sequence specificity using a DNA ligase disclosed herein. In certain embodiments, the DNA ligase ligates the two ends at one nick of the hairpin-ended DNA molecule disclosed herein (e.g., hairpin-ended DNA molecule described in Section 5.1.1), thereby repairing said one nick. In certain embodiments, the DNA ligase ligates each pair of two ends at the two nicks of the hairpin-ended DNA molecule (e.g., hairpin-ended DNA molecule described in Section 5.1.1), thereby repairing the two nicks. In certain embodiments, the DNA ligase ligates each pair of two ends at all nicks of the hairpin-ended DNA molecule (e.g., hairpin-ended DNA molecule described in Section 5.1.1), thereby repairing all nicks of the hairpin-ended DNA molecule. In certain embodiments, the hairpin-ended DNA molecule comprises two nicks. In certain embodiments, the hairpin-ended DNA molecule consists of one nick. In certain embodiments, the hairpin-ended DNA molecule comprises one nick.

In certain embodiments, the step of repairing the nicks with a ligase can be performed according to the incubation conditions as known and practiced in the art.

Various ligases known and used in the art can be used in the methods provided herein. An exemplary list of ligases provided as embodiments for the ligases for use in the methods are described in the catalog of New England Biolabs, which is available at neb.com/products/dna-modifying-enzymes-and-cloning-technologies/dna-ligases/dna-ligases and incorporated herein in its entirety by reference. The conditions for the various ligases to digest the DNA molecules are known for the various ligases provided herein, including the temperatures, the salt concentration, the pH, the buffering reagent, the presence or absence of certain detergent, and the duration of incubation to achieve the desired percentage of digestion. These conditions are readily available from the websites or catalogs of various vendors of the restriction enzymes, e.g., New England BioLabs. The ligation conditions also correlates with the freedom of movement of the two DNA ends to be ligated. When the two DNA ends can be brought to proximity or can have a higher probability of coming to proximity of each other, for example by both ends annealing to a common DNA strand, ligation can be enhanced.

In certain embodiments, methods disclosed herein can be used to generate hairpin-ended DNA molecules at high scale, high yield, and/or high purity. In certain embodiments, high scale, high yield, and/or high purity can be accomplished in a single reaction vessel. In certain embodiments, the high scale is at least 1 mg, 10 mg, 100 mg, 1 g, 10 g, 100 g, 1 kg, or at least 10 kg. In certain embodiments, the high yield is at least 50%, 60%, 70%, 80%, 90%, 95%, 98%, or at least 99% yield (comparing number of plasmid copies used as input and number of hairpin-ended DNA molecules as product). In certain embodiments, the high purity is at least 50%, 60%, 70%, 80%, 90%, 95%, 98%, or at least 99% purity of hairpin-ended DNA molecules as product as a result of a method provided herein.

### 5.3.6 Additional Steps

Similar to Section 5.2.5, additional steps can be performed after the conclusion of making hairpin-ended DNA molecules (e.g., after the step of annealing as described in Section 5.3.4 or after the step of repairing the nicks as described in Section 5.3.5) and before the initiation of removing undesired DNA molecules (e.g., before the step of generating double strand breaks for exonuclease as described in Section 5.4.1 or before the step of digestion with exonuclease as described in Section 5.4.2).

In certain embodiments, an additional step is performed to prepare the reaction mixture and optimize the reaction conditions for the subsequent step of removing undesired DNA molecules (e.g., generating double strand breaks for exonuclease as described in Section 5.4.1 or digestion with exonuclease as described in Section 5.4.2).

In certain embodiments, the additional steps comprise buffer exchange, which replaces the buffer with a buffer for the enzymes used in the next step (e.g., the restriction enzymes and nicking endonucleases described in Section 5.4.1, or the exonuclease described in Section 5.4.2). In certain embodiments, the additional steps comprise removing the nicking endonuclease and/or or ligase from the reaction mixture. In certain embodiments, the additional steps are performed using tangential flow filtration (TFF). In certain embodiments, an additional step is added for industrial scale production of the hairpin-ended DNA molecules.

In certain embodiments, both steps of making hairpin-ended DNA molecules and removing undesired DNA molecules are carried out in the same container or reaction mixture (e.g., one pot reaction). In certain embodiments, a buffer or a reagent(s) is added to the reaction mixture at the end of the annealing or ligation to provide conditions for the restriction enzymes and nicking endonucleases described in Section 5.4.1 or the exonuclease described in Section 5.4.2 to work.

In certain embodiments, the reactions are carried out continuously, where no additional steps (e.g., buffer exchange, removing molecules from the reaction mixture, concentrating DNA molecules) are included between the conclusion of the making hairpin-ended DNA molecules and initiation of removing undesired DNA molecules.

### 5.4 Removal of Undesired DNA Molecules

In certain embodiments, methods disclosed herein further comprise removing undesired DNA molecules from the reaction after the hairpin-ended DNA molecules are generated. Such a step improves the purity of the hairpin-ended DNA molecules of interest (i.e., hairpin-ended DNA molecules comprising the sequence of interest) and produces transfection/transcription-ready hairpin-ended DNA molecules of interest.

The remaining undesired DNA molecules can be hydrolyzed using an exonuclease as disclosed in Section 5.4.2 or be cleaved or nicked by restriction enzyme or nicking endonuclease to generate double strand breaks for exonuclease digestion as disclosed in Section 5.4.1.

In certain embodiments, the remaining undesired DNA molecules can be reduced in molecular weight (e.g. fragmented) by cleavage or nicking with a restriction enzyme or nicking endonuclease to generate double strand breaks for separation by molecular weight exonuclease digestion as disclosed in Section 5.4.1.

### 5.4.1 Generating Double Strand Breaks for Exonuclease Digestion

In certain embodiments, the remaining undesired DNA molecules are undesired hairpin-ended DNA molecules, which do not comprise the sequence of interest. In certain embodiments, the remaining undesired DNA molecules do not comprise a double strand break that can serve as a substrate for an exonuclease. Methods disclosed herein further comprises incubating the undesired hairpin-ended DNA molecules with a restriction enzyme or a nicking endonuclease to generate a double strand break for subsequent exonuclease digestion. The restriction enzyme or nicking endonuclease incubation creates double strand breaks and generates non-hairpin-ended DNA molecules (i.e., DNA molecules comprises at least one non-hairpin end) for exonuclease digestion.

In certain embodiments, the undesired hairpin-ended DNA molecule (i.e., hairpin-ended DNA molecules not comprising a sequence of interest) comprise a restriction enzyme site as described in Section 5.1.3. Various restriction enzymes known and used in the art can be used in the methods provided herein. An exemplary list of restriction enzymes provided as embodiments for the restriction enzymes for use in the methods and the corresponding restriction sites for the restriction enzymes are described in the catalog of New England Biolabs, which is available at neb.com/products/restriction-endonucleases and incorporated herein in its entirety by reference. The conditions for the various restriction enzymes to cleave the dsDNA are known for the various restriction enzymes provided herein, including the temperatures, the salt concentration, the pH, the buffering reagent, the presence or absence of certain detergent, and the duration of incubation to achieve the desired percentage of nicked DNA molecules. These conditions are readily available from the websites or catalogs of various vendors of the restriction enzymes, e.g., New England BioLabs. The disclosure provides that the step of incubating the DNA molecule with the restriction enzymes is performed according to the incubation conditions as known and practiced in the art.

In certain embodiments, the undesired hairpin-ended DNA molecule (i.e., hairpin-ended DNA molecules not comprising a sequence of interest) comprises an MSRE site as described in Section 5.1.3(a). Exemplary MSREs and the reaction conditions that can be used with the presently disclosed methods are disclosed in Section 5.2.4.

In certain embodiments, the undesired hairpin-ended DNA molecule (i.e., hairpin-ended DNA molecules not comprising a sequence of interest) comprises two nicking endonuclease sites as described in Section 5.1.4. Exemplary nicking endonucleases (including programmable nicking enzymes) and the reaction conditions that can be used with the presently disclosed methods are disclosed in Section 5.3.2.

In certain embodiments, the undesired hairpin-ended DNA molecule (i.e., hairpin-ended DNA molecules not comprising a sequence of interest) comprises an MSNE site as described in Section 5.1.4(a). Exemplary MSREs and the reaction conditions that can be used with the presently disclosed methods are disclosed in Section 5.2.4.

5.4.2 Incubating with Exonucleases

Methods disclosed herein further comprise incubating with an exonuclease. In general, the desired product is a hairpin-ended DNA molecule. Such hairpin-ended DNA molecules are typically resistant to digestion with an exonuclease. As such, any DNA fragments that are not the desired hairpin-ended DNA molecules, can be eliminated by digestion with an exonuclease because incubation with an exonuclease selectively digests the DNA molecules with a double strand break while leaving hairpin-ended DNA molecules intact. In certain embodiments, the remaining undesired DNA molecules comprise undesired hairpin-ended DNA molecules, which do not comprise correctly annealed inverted repeats and are, thus, sensitive to exonuclease digestion. In certain embodiments, the remaining undesired DNA molecules comprise undesired hairpin-ended DNA molecules, which comprise parts or fragments of the sequence of interest and/or double strand breaks. An advantage is that further purification steps for the separation of DNA molecules from each other can be reduced or avoided. In certain embodiments, upon completion of a method described herein, the resulting hairpin-ended DNA molecule is essentially transfection and transcription-ready without further processing. In certain embodiments, upon completion of a method described herein, the resulting hairpin-ended DNA molecule is essentially transfection and transcription-ready with only a single precipitation followed by reconstitution in transfection buffer.

In certain embodiments, a method as described herein results in hairpin-ended DNA molecules of interest but also other hairpin-ended DNA molecules that would not be substrate for exonuclease digestion. In certain embodiments, the amplification product is a tandem repeat of a DNA sequence. Upon generation of hairpin-ended DNA molecules from that tandem repeat, hairpin-ended DNA molecules of interest are generated but the DNA fragments between the DNA fragments that are processed to become hairpin-ended DNA molecules of interest would also be hairpin-ended. These DNA fragments (i.e., undesired hairpin-ended DNA molecules, which do not comprise a sequence of interest) can become substrates for exonucleases upon digestion with restriction endonucleases whose recognition sites are only present in these undesired hairpin-ended DNA molecules but not in the hairpin-ended DNA molecules of interest (see Section 5.4.1). Similarly, double strand breaks in these undesired hairpin-ended DNA molecules can be generated by two nicking endonucleases on opposite strands (see Section 5.4.1).

Exonucleases cleave nucleotides from the end (exo) of a DNA molecule. Exonucleases can cleave nucleotides along the 5′ to 3′ direction, along the 3′ to 5′ direction, or along both directions. In certain embodiments, an exonuclease for use in the methods provided herein cleaves nucleotides with no sequence specificity. In certain embodiments, an exonuclease for use in the methods provided herein digests the DNA fragments comprising ends created by one or more nicking endonuclease recognizing and nicking the additional nicking endonuclease sites disclosed in Section 5.1.3 (e.g., fifth and sixth restriction sites) or by a restriction enzyme recognizing and cleaving the restriction enzyme sites disclosed in Section 5.1.4 presented in amplification products or undesired DNA molecules.

Various exonucleases known and used in the art can be used in the methods provided herein. An exemplary list of exonucleases provided as embodiments for the restriction enzymes for use in the methods are described in the catalog of New England Biolabs, which is available at neb.com/products/dna-modifying-enzymes-and-cloning-technologies/nucleases and incorporated herein in its entirety by reference. The conditions for the various exonucleases to digest the DNA molecules are known for the various exonucleases provided herein, including the temperatures, the salt concentration, the pH, the buffering reagent, the presence or absence of certain detergent, and the duration of incubation to achieve the desired percentage of digestion. These conditions are readily available from the websites or catalogs of various vendors of the restriction enzymes, e.g. New England BioLabs. The step of incubating the DNA molecule with the restriction enzymes is performed according to the incubation conditions provided by the manufacturer.

In certain embodiments, an exonuclease for use in the methods provided herein can be an exonuclease that selectively digests DNA molecules with one or more double strand breaks, while leaving intact the circular ssDNA/dsDNA molecules or DNA molecules comprising one or more nicks but no ends. In certain embodiments, the exonuclease is Exonuclease V (RecBCD). In certain embodiments, the exonuclease is Exonuclease VIII or truncated Exonuclease VIII. In certain embodiments, the exonuclease is Exonuclease I. Exonuclease V (RecBCD), Exonuclease VIII, Exonuclease I, and truncated Exonuclease VIII comprise the selectivity described in this paragraph. In certain embodiments, two or more different exonucleases are used. In certain embodiments, at least one exonuclease cleaves nucleotides along the 5′ to 3′ direction while at least one exonuclease cleaves nucleotides along the 3′ to 5′ direction. In certain embodiments, both Exonuclease V and truncated Exonuclease VII are used. In certain embodiments, the two or more different exonucleases are added concurrently or sequentially.

In certain embodiments, generation of hairpin-ended DNA molecules with a portion of single strand DNA is desired. To generate such DNA molecules, exonucleases can be used that selectively digest linear segments of DNA molecules, initiating from one or more nicks, but which cannot progress through folded hairpins, terminating the digestion at the hairpin and leaving a ssDNA behind. In certain embodiments, an exonuclease for use to initiate at one or more nicks and/or double strand break can be a T7 exonuclease. Other suitable exonucleases are also known, used in the art, and provided herein, for example, as described on the websites or in the catalogs of various vendors of exonucleases including New England BioLabs.

In certain embodiments, after exonuclease treatment, the hairpin-ended DNA molecules of the present disclosure are substantially free of any sequences of the DNA template that was used for the amplification reaction. Specifically, if the DNA template originated from a prokaryotic plasmid, after exonuclease treatment, the hairpin-ended DNA molecules of the present disclosure are substantially free of any prokaryotic backbone sequences. In certain embodiments, the backbone refers to the plasmid sequence that is not part of the sequence encompassing the sequence of interest in between the two ITRs. In certain embodiments, the backbone refers to the vector sequence that is not part of the sequence encompassing the sequence of interest in between the two ITRs. In certain embodiments, the DNA molecules of the disclosure are 100% free, 99% free, 98% free, 97% free, 96% free, 95% free, 94% free, 93% free, 92% free, 91% free, or 90% free of prokaryotic backbone sequence of the parental plasmid of the DNA template.

In certain embodiments, at least 50%, 60%, 70%, 75%, 80%, 85%, 90%, 95%, 96%, 97%, 98%, or at least 99% of the hairpin-ended DNA molecules remain intact during the step of incubation with an exonuclease. In certain embodiments, 100% of hairpin-ended DNA molecules remain intact during the step of incubation with an exonuclease.

Following digestion with an exonuclease, the hairpin-ended DNA molecules provided herein are essentially transfection ready for further uses (see Section 5.5, e.g., transfection into cells for correcting genes or production of therapeutic proteins or RNAs, in vitro transcription or translation for production of mRNA or viral particles). In some embodiments, the reaction mixture following the incubation with an exonuclease is subjected to a DNA precipitation and/or chromatography to further purify the hairpin-ended DNA molecules of interest. The purified DNA can be washed and reconstituted in appropriate buffer by any method known to the skilled artisan. DNA precipitation can be conducted in ethanol or any other method known to the skilled artisan.

### 5.4.3 Generating Double Strand Breaks for Molecular Weight Based Separation

In certain embodiments, the methods disclosed herein further comprise physically separating hairpin-ended DNA molecules from undesired non-hairpin-ended DNA molecules. The restriction enzyme or nicking endonuclease incubation as outlined in Section 5.4.1 can also create double strand breaks to generate lower molecular weight non-hairpin-ended DNA molecules (i.e., DNA molecules comprising at mostat least one non-hairpin end) for separation by size. In certain embodiments, the double strand breaks are located on the undesired DNA molecules in such a way that cleavage by the restriction enzyme(s) or nicking endonuclease results in fragments comprising between 50 bp and 1,500 bp. DNA fragments within this size range can be efficiently removed by size exclusion chromatography or TFF, thus improving the process of removing undesired DNA molecules and increasing the purity of the hairpin-ended DNA molecules.

In one embodiment, where a reaction mixture comprises desired hairpin-ended DNA molecules and a undesired DNA molecules comprising double strand breaks, or fragments thereof having lower molecular weight, the method comprises purifying the desired hairpin-ended DNA molecules by removing the undesired DNA molecules, or fragments thereof. In one embodiment, the removal of the undesired DNA molecules comprises use of core bead flow-through chromatography. Without wishing to be bound by theory, use of core bead flow-through chromatography allows the hairpin-ended DNA molecules to be recovered after elution and to have as much of the undesired DNA molecules, or fragments thereof, to be retained inside the chromatographic material (such as inside the beads), leading to a separation.

In certain embodiments, after purification by size exclusion chromatography, the purified hairpin-ended DNA molecules are transfection ready (e.g., ready for use as described in Section 5.5). In some embodiments, the material obtained after purifying by size exclusion chromatography is further purified by incubation with an exonuclease, optionally followed by DNA precipitation and/or chromatography. The purified DNA can be washed and reconstituted in appropriate buffer by any method known to the skilled artisan. DNA precipitation can be conducted in ethanol or any other solvent known to the skilled artisan.

### 5.5 Use of Hairpin-ended DNA Molecules

Hairpin-ended DNA molecules made by the methods disclosed herein and compositions comprising thereof have a wide variety of applications. Importantly, the presently disclosed methods and compositions provide a scalable, high-fidelity, high-yield, and high-purity way of generating hairpin-ended DNA molecules. In certain embodiments, the generated hairpin-ended DNA molecules are transfection ready. In other words, once generated according to the methods disclosed herein, the hairpin-ended DNA molecules and compositions comprising thereof can be used to transfect biological cells in vivo (e.g., administered to animals or patients) or in vitro (e.g., added to a cell culture) without further processing (e.g., purification and/or modification. In certain embodiments, hairpin-ended DNA molecules comprising two nicks as described in Section 5.1.1(d) are transfection ready.

The ultimate use of a hairpin-ended DNA is determined by the sequence of interest comprised by the hairpin-ended DNA. Exemplary sequences of interest that can be used with the present disclosure are set forth in Section 5.1.1(c).

In one aspect, provided herein is a method of producing AAV vectors for use in gene therapy. In certain embodiments, the method comprises (a) providing a circular DNA molecule as a template (e.g., as described in Section 5.1); (b) amplifying the template (e.g., as described in Section 5.2) to produce at least one amplification product (as described in Section 5.1); (c) processing the at least one amplification product to generate at least one hairpin-ended DNA molecule (e.g, as described in Section 5.3) encoding a transgene product; (d) transfecting a host cell with the at least one hairpin-ended DNA molecule for production of the transgene product (e.g. according to methods known in the art); and (e) harvesting the transgene product (e.g. according to methods known in the art). In certain embodiments, the template is amplified in step b by rolling circle amplification. In certain embodiments, the method further comprises before step (d) removing undesired DNA molecules (e.g., non-hairpin-ended) from the reaction mixture (e.g., as described in Section 5.4) resulting from step (c). In one embodiment, the transgene product is a component of a viral vector, a replication associated protein, and/or a helper plasmid. In one embodiment, the component of the viral vector is a viral vector genome and/or a capsid protein. In one embodiment, the transgene product is a component of a Cas/CRISPR system. In certain embodiments, the hairpin-ended DNA molecule is an exemplary hairpin-ended DNA molecule described in Section 5.1.1(d).

In certain embodiments, hairpin-ended DNA molecules provided herein can be used for gene therapy. For example, hairpin-ended DNA molecules provided herein can be packaged in a viral vector (such as an AAV capsid) for gene therapy. In another example, hairpin-ended DNA molecules provided herein can be used to produce viral vector genome (such as AAV vector genome) that can be packaged in a viral capsid for gene therapy. In certain embodiments, hairpin-ended DNA molecules comprising two nicks as described in Section 5.1.1(d) are suitable for use in producing viral vector genome (such as AAV vector genome) in a host cell, wherein the viral vector genome can be packaged in a viral capsid for gene therapy. Any genetic material (e.g., sequence of interest disclosed in Section 5.1.1(c)) can be carried by the hairpin-ended DNA molecule and then shuttled via the viral vector into a cell.

In certain embodiments, the hairpin-ended DNA are suitable for AAV vector production. In one aspect, provided herein is a method of producing AAV vectors for use in gene therapy. In certain embodiments, the method comprises (a) providing a circular DNA molecule as a template (e.g., as described in Section 5.1); (b) amplifying the template (e.g., as described in Section 5.2) to produce at least one amplification product (as described in Section 5.1); (c) processing the at least one amplification product to generate at least one hairpin-ended DNA molecule (e.g, as described in Section 5.3) encoding an AAV vector genome, wherein the AAV vector genome comprises a 5′ ITR, an expression cassette, and a 3′ ITR; (d) transfecting a host cell with the at least one hairpin-ended DNA molecule for production of AAV particles; and (e) harvesting the AAV particles. In certain embodiments, the method further comprises before step (d) removing undesired DNA molecules (e.g., non-hairpin-ended) from the reaction mixture (e.g., as described in Section 5.4) resulting from step (c).

In certain embodiments, step (d) comprises co-transfecting the host cell with (i) the hairpin-ended DNA molecule encoding the AAV vector genome and (ii) one or more DNA molecules encoding Rep protein(s), AAV capsid protein(s), and/or helper plasmid(s). In certain embodiments, the DNA molecules of (i) and (ii) only encode the AAV vector genome and helper plasmid, and the Rep and/or AAV capsid proteins are present in the host cell. In certain embodiments, step (d) comprises co-transfecting the host cell with (i) the hairpin-ended DNA molecule encoding the AAV vector genome and (ii) one or more hairpin-ended and/or circular DNA molecules encoding Rep proteins, AAV capsid proteins, and/or helper plasmids. In certain embodiments, step (d) comprises co-transfecting the host cell with (i) the hairpin-ended DNA molecule encoding the AAV vector genome; (ii) a hairpin-ended and/or circular DNA molecule encoding Rep proteins and AAV capsid proteins; and (iii) a hairpin-ended and/or circular DNA molecule encoding helper plasmids. In certain embodiments, step (d) comprises co-transfecting the host cell with (i) the hairpin-ended DNA molecule encoding the AAV vector genome; (ii) a hairpin-ended DNA molecule encoding Rep proteins and AAV capsid proteins; and (iii) a hairpin-ended DNA molecule encoding helper plasmids. In certain embodiments, step (d) comprises co-transfecting the host cell with (i) the hairpin-ended DNA molecule encoding the AAV vector genome; (ii) a plasmid encoding Rep proteins and AAV capsid proteins; and (iii) a plasmid encoding helper plasmids. In certain embodiments, the AAV capsid proteins are capsid proteins of any known AAV serotype, for example AAV2, AAV4, AAV5, AAV6, AAV8, or AAV9. In certain embodiment, the hairpin-ended DNA molecules are one or more exemplary hairpin-ended DNA molecules described in Section 5.1.1(d).

In certain embodiments, hairpin-ended DNA molecules can be used for delivering components of viral genomes (e.g., components of helper plasmids, components of AAV genome, components of lentiviral genome, or components of adenoviral genome, sequences encoding Rep and Cap of AAV vectors) to a host cell for production of viral particles.

In some embodiments, the genetic material (e.g., sequence of interest disclosed in Section 5.1.1(c)) needs to be first transcribed to be packaged into a retrovirus vector (e.g. lentivirus). Thus, the hairpin-ended DNA contains the elements known in the art to make the genetic material suitable for the transcription, processing, and packaging. In certain embodiments, hairpin-ended DNA molecules comprising two nicks as described in Section 5.1.1(d) are suitable for use in producing a retrovirus vector (e.g. lentivirus).

In certain embodiment, the hairpin-ended DNA are suitable for lentiviral vector production. In one aspect, provided herein is a method of producing lentiviral vectors for use in gene therapy. In certain embodiments, the method comprises (a) providing a circular DNA molecule as a template (e.g., as described in Section 5.1); (b) amplifying the template (e.g., as described in Section 5.2) to produce at least one amplification product (as described in Section 5.1); (c) processing the at least one amplification product to generate at least one hairpin-ended DNA molecule (e.g., as described in Section 5.3) encoding a lentiviral transfer vector, wherein the lentiviral transfer vector comprises 5′ long terminal repeat (LTR) sequence, an expression cassette, and a 3′ LTR; (d) transfecting a host cell with the at least one hairpin-ended DNA molecule for production of lentiviral particles; and (e) harvesting the lentiviral particles. In certain embodiments, the method further comprises before step (d) removing undesired DNA molecules (e.g., non-hairpin-ended) from the reaction mixture (e.g., as described in Section 5.4) resulting from step (c). In certain embodiment, the hairpin-ended DNA molecule is one or more exemplary hairpin-ended DNA molecule described in Section 5.1.1(d). In certain embodiments, the lentiviral transfer vector comprises in order from 5′ to 3′: a first IR, a 5′ LTR, an expression cassette, a 3′ LTR, and a second IR. In certain embodiments, the lentiviral transfer vector comprises in order from 5′ to 3′: a first IR, a first nick, a 5′ LTR, an expression cassette, a 3′ LTR, a second nick, and a second IR. In certain embodiments, the lentiviral transfer vector comprises in order from 5′ to 3′: a 5′ LTR, an expression cassette comprising a promoter operably linked to an ORF, a Woodchuck HBV Posttranscriptional regulatory element (WPRE), a 3′ LTR, and a poly (A) signal sequence. In certain embodiments, the lentiviral transfer vector comprises in order from 5′ to 3′: a hybrid 5′ LTR, an expression cassette comprising a promoter operably linked to an ORF, a WPRE, a 3′ self-inactivating (SIN) LTR, and a poly (A) signal sequence. In certain embodiments, the lentiviral transfer vector comprises in order from 5′ to 3′: a 5′ LTR, an expression cassette comprising a promoter operably linked to an ORF, a 3′ LTR, and a poly (A) signal sequence. In certain embodiments, the lentiviral transfer vector comprises in order from 5′ to 3′: a hybrid 5′ LTR, an expression cassette comprising a promoter operably linked to an ORF, a 3′ self-inactivating (SIN) LTR, and a poly (A) signal sequence.

In certain embodiments, step (d) comprises co-transfecting the host cell with (i) the hairpin-ended DNA molecule encoding the lentiviral transfer vector and (ii) one or more DNA molecules encoding packaging and/or envelope proteins (e.g. VSV-G protein(s)), Tat proteins, Rev protein(s), Gag protein(s), and/or Pol protein(s). In certain embodiments, step (d) comprises co-transfecting the host cell with (i) the hairpin-ended DNA molecule encoding the lentiviral transfer vector and (ii) one or more hairpin-ended and/or circular DNA molecules encoding packaging and/or envelope proteins (e.g. VSV-G protein(s)), Tat proteins, Rev protein(s), Gag protein(s), and/or Pol protein(s).

In certain embodiments, step (d) comprises co-transfecting the host cell with (i) the hairpin-ended DNA molecule encoding the lentiviral transfer vector; (ii) a hairpin-ended and/or circular DNA molecule encoding Rev, Gag, Pol, and Tet proteins; and (iii) a hairpin-ended and/or circular DNA molecule encoding VSV-G protein. In certain embodiments, step (d) comprises co-transfecting the host cell with (i) the hairpin-ended DNA molecule encoding the lentiviral transfer vector; (ii) a hairpin-ended and/or circular DNA molecule encoding Rev protein; (iii) a hairpin-ended and/or circular DNA molecule encoding Gag and Pol proteins; and (iv) a hairpin-ended and/or circular DNA molecule encoding VSV-G protein. In certain embodiments, step (d) comprises co-transfecting the host cell with (i) the hairpin-ended DNA molecule encoding the lentiviral transfer vector; (ii) a hairpin-ended DNA molecule encoding Rev protein; (iii) a hairpin-ended DNA molecule encoding Gag and Pol proteins; and (iv) a hairpin-ended DNA molecule encoding VSV-G protein. In certain embodiments, expression of transgene product from the lentiviral transfer vector is Tat dependent. In certain embodiments, expression of transgene product from the lentiviral transfer vector is Tat independent. In certain embodiment, the hairpin-ended DNA molecules are one or more exemplary hairpin-ended DNA molecules described in Section 5.1.1(d).

In some embodiments, the production of lentivirus vectors comprises a set of hairpin-ended DNA molecules that comprise at least one lentiviral transfer vector and at least one lentiviral production vector encoding packaging genes, envelope genes, the Tat gen, and the Rev gene. In some embodiment, the method of producing lentivirus vectors comprises use of a single packaging hairpin-ended DNA molecule encoding the Gag, Pol, Rev, and Tat genes, an envelope hairpin-ended DNA molecule encoding VSVg, and a transfer hairpin-ended DNA molecule where transgene expression from the 5′ wild type LTR is Tat-dependent. In some embodiments, the method of producing lentivirus vectors comprises (i) a transfer hairpin-ended DNA molecule where both LTRs are used (both 5′ and 3′) and the dependence on Tat is removed, (ii) a hairpin-ended DNA molecule encoding an envelope protein, (iii) a hairpin-ended DNA molecule encoding the packaging genes Gag and Pol, and (iv) a hairpin-ended DNA molecule encoding the Rev gene. In a preferred embodiment, the envelope protein is a Vesicular Stomatitis Virus Glycoprotein (VSV-G). In some embodiments, a subset of all vectors co-transfected into the host cell in step (d) are hairpin-ended DNA molecules, and the rest of the vectors are circular DNA molecules. In some embodiments, a subset of all vectors co-transfected into the host cell in step (d) are hairpin-ended DNA molecules, and the rest of the vectors are plasmid vectors or minicircles. In one embodiment, all vectors co-transfected into the host cell in step (d) are hairpin-ended DNA molecules.

The transgene product and/or viral vectors may be produced in producing cells. In certain embodiments, the host cell is a producing cell. In some embodiments, the producing cells are permissive cells. In some embodiments, the producing cells are mammalian cells. In some embodiments, the producing cells are Chinese Hamster Ovary (CHO) cells. In a preferred embodiment, the producing cells are human embryonic kidney cells (HEK293 cells) or a variant thereof. In another preferred embodiment, the producing cells are HEK293T cells, HEK293F or other HEK293 cell for suspension culture. The producing cells can be cultured in a serum-free medium selected with respect to the specific cell used and/or which permits the production of the viral vectors. The person skilled in the art will be able to select the appropriate media with respect to the mammalian host cells. The medium may be supplemented with additives such as a non-ionic surfactant such as Pluronic® F68 (Invitrogen, catalogue No. 24040-032), used for controlling shear forces in suspension cultures, an anti-clumping agent (e.g. from Invitrogen, catalogue No. 0010057AE) and/or L-glutamine or an alternative to L-glutamine such as a L-alanyl-L-glutamine dipeptide, e.g. GlutaMAX™ (Invitrogen, catalogue No 35050-038). In some embodiments, the producing cells are stable cell lines expressing some of the accessory/helper functions required for viral production. In some embodiments, the producing cells are stable cell lines expressing the Adenovirus helper functions for AAV vector production. In some embodiments, the producing cells are stable cell lines expressing the Rep and/or Cap proteins for AAV vector production.

The hairpin-ended DNA molecules used for transgene product and/or viral vector production as described herein may be introduced to the host cells (e.g. producing cells) in step (d) by suitable means, such as transfection or electroporation. In some embodiments, the transfection is a chemical transfection. In some embodiments, the chemical transfection comprises use of calcium phosphate (CaPO₄) as a transfection agent. In some embodiments, the transfection is a liposome-based transfection. In some embodiments, the transfection is a polymer-based transfection. In some embodiments, the polymer-based transfection comprises use of polyethylnimine (PEI) as a transfection agent. In some embodiments, the polymer-based transfection comprises use of polyethylnimine (PEIpro) as a transfection agent. In some embodiments, the transfection comprises use of Fecto VIR-AAV as a transfection agent. In some embodiments, the transfection comprises use of Fecto VIR-LV as a transfection agent. In some embodiments, the transfection comprises use of jetOPTIMUS as a transfection agent. In some embodiments, the transfection comprises use of jetPRIME as a transfection agent.

In certain embodiments, the host cell comprises an inducible system for the production of transgene product or viral vector particles (e.g. AAV or lentiviral particles). In certain embodiments a host cell transfected with the hairpin-ended DNA molecule encoding the transgene or viral vector components (e.g. viral vector genome) may be induced to begin production of the viral vector particle (e.g. AAV or lentiviral vector). Inducible systems are well known in the art, for example Tet-on and Tet-off systems, which are based on the addition or removal, respectively of the tetracycline/doxycycline antibiotic in the culture medium to trigger gene transcription through the tetracycline response element (TRE). Alternative inducible systems include, but are not limited to, Tet-on/cumate inducible system and ecdysone inducible system. In other embodiments, the host cell comprises a constitutive system for the production of viral vector particles (e.g. AAV or lentiviral particles).

In certain embodiments, before harvesting in step (e), the host cell culture of step (d) is allowed to grow for between 24 and 72-hours after transfection. In some embodiments, the host cell culture of step (d) is grown between 48 and 72-hours after transfection. Methods for culturing the transfected host cell are known in the art. Said methods of culturing include but are not limited to the use of various cell culture media, appropriate gas concentration/exchange and/or temperature control means to promote growth of the cells and/or integration of the constructs into the genome of the cell.

In certain embodiments, step (e) comprises harvesting the transgene product or viral vector particles using one or more standard harvesting techniques known in the art. In certain embodiments, after step (e), the harvested transgene product or viral vector particles are purified using standard techniques known in the art. In certain embodiments, the total particle, infectious, and/or genomic titres of the harvested and/or purified viral vector particles can be determined by standard methods known in the art.

In certain embodiments, hairpin-ended DNA molecules can be used for CRISPR-mediated Homology Directed Repair (HDR). In certain embodiments, the sequence of interest comprises a nucleotide sequence encoding a component of the CRISPR/Cas system (e.g., guided RNA, RNA guided endonuclease). In certain embodiments, the sequence of interest comprises a synthetic DNA sequence to be incorporated into the host cell genome by CRISPR-mediated HDR.

In certain embodiments, hairpin-ended DNA molecules are used for RNA therapies. In certain embodiments, the sequence of interest of the hairpin-ended DNA molecule encodes an RNAi, an anti-sense RNA, an siRNA, an shRNA, an sr-RNA, a mimic of a microRNA, an anti-microRNA, and/or a mRNA.

In certain embodiments, hairpin-ended DNA molecules are used for in vitro transcription (IVT) of mRNA. The generated mRNA transcripts can be further used for producing proteins (e.g., therapeutic proteins) in cells. In certain embodiments, the sequence of interest of the hairpin-ended DNA molecule comprises a nucleotide sequence encoding the mRNA. In certain embodiments, the sequence of interest comprises a posttranscriptional regulatory element. In certain embodiments, the sequence of interest further comprises a polyadenylation and/or termination signal. In certain embodiments, the polyadenylation is directly encoded in the sequence of interest as a homopolymer such that the mRNA is directly synthesized with a polyadenylation sequence. In certain embodiments, the hairpin-ended DNA is nicked or digested at the end of the polyadenylation sequence before IVT. In certain embodiments, the hairpin-ended DNA is nicked or digested at the end of IVT to terminate the production of mRNA.

In certain embodiments, the hairpin-ended DNA molecules provided herein may be suitable for the production of RNA (e.g. mRNA, including modified mRNA molecules and self-amplifying RNA (saRNA)). In one aspect, provided herein is a method of producing RNA. In certain embodiments, the method of producing RNA comprises (a) providing a circular DNA molecule as a template (e.g., as described in Section 5.1); (b) amplifying the template (e.g., as described in Section 5.2) to produce at least one amplification product (as described in Section 5.1); (c) processing the at least one amplification product to generate at least one hairpin-ended DNA molecule (e.g, as described in Section 5.3), wherein the hairpin-ended DNA molecule comprises a transcription unit suitable for IVT; (d) transcribing the hairpin-ended DNA molecule, or a fragment thereof, for production of RNA (e.g. according to methods known in the art, including IVT); and (e) harvesting the RNA product (e.g. according to methods known in the art). In certain embodiments, the method further comprises before step (d) removing undesired DNA molecules (e.g., non-hairpin-ended) from the reaction mixture (e.g., as described in Section 5.4) resulting from step (c). In certain embodiments, the hairpin-ended DNA molecule is an exemplary hairpin-ended DNA molecule described in Section 5.1.1(d).

In certain embodiments, the hairpin-ended DNA molecule comprises an expression cassette (e.g., as described in Section 5.1.1(c)(i)) comprising at least one transcription unit suitable for IVT. In certain embodiments, the hairpin-ended DNA molecule comprising the at least one transcription unit suitable for IVT further comprises two nicks as described in Section 5.1.1(d). In certain embodiments, the transcription unit comprises an ORF operably linked to at least one RNA polymerase promoter. In some embodiments, the transcription unit comprise an ORF operably linked to at least one, at least two, or at least three promoters selected from the bacteriophage SP6, T7 or T3 RNA polymerases, or any combination thereof. In some embodiments, at least one of the RNA polymerase promoters has been modified for use with the initiating sequence 5′ AG 3′.

In some embodiments, the transcription unit comprises an RNA polymerase promoter sequence operably linked to a nucleotide sequence encoding an RNA polymerase. In certain embodiments, the nucleotide sequence encoding the RNA polymerase is further operably linked to a subgenomic promoter. In certain embodiments, the subgenomic promoter is further operably linked to an ORF. For example, in a preferred embodiment, the transcription unit comprises an RNA-dependent RNA polymerase (RdRp) promoter sequence operably linked to a nucleotide sequence encoding RdRp, which is operably linked to a subgenomic promoter, which is operably linked to an ORF. In some embodiments, the transcription unit comprises an RNA-dependent RNA polymerase (RdRp) promoter sequence, operably linked to a subgenomic promoter, which is operably linked to an ORF. In some embodiments, the transcription unit is flanked by 5′ and 3′ CSE sequences.

In certain embodiments, the transcription unit further comprises features to direct the cellular machinery to make RNA and protein. In certain embodiments, the transcription unit comprises regulatory elements known and used in the art to regulate (e.g., promote, inhibit and/or turn on/off) the expression of the ORF). Such regulatory elements include, for example, an 5′-untranslated region (UTR), 3′-UTR, or both the 5′UTR and the 3′UTR. In some embodiments, the hairpin-ended DNA molecule comprises an expression cassette (as disclosed in section 5.1.1(c)(i)) comprising a transcriptional unit suitable for IVT, wherein the transcription unit comprises an RNA polymerase promoter sequence operably linked to an ORF, wherein the transcriptional unit further comprises a 5′ untranslated region (UTR) and/or a 3′ UTR. In some embodiments, the hairpin-ended DNA molecule comprises an expression cassette (as disclosed in section 5.1.1(c)(i)) comprising a transcriptional unit suitable for IVT, wherein the transcription unit includes an RNA polymerase promoter sequence operably linked to a nucleotide sequence encoding an RNA polymerase, which is operably linked to a subgenomic promoter, which is operably linked to an ORF, wherein the transcriptional unit further comprises a 5′ untranslated region (UTR) and/or a 3′ UTR.

In some embodiments, the transcription unit further comprises an intron. In certain embodiments, the intron is encoded upstream of the ORF of the hairpin-ended DNA molecule. In some embodiments, the intron is encoded within the ORF.

Without wishing to be bound by theory, cap-dependent translation involves recruitment of the pre-initiation complex (PIC) to the 5′ end of an mRNA followed by scanning to find an AUG initiation codon in an optimum sequence context. AUG recognition promotes scanning cessation, release of most initiation factors, and recruitment of the large ribosomal subunit to initiate elongation. Efficient recognition of an initiation codon depends on its surrounding sequence. In a preferred embodiment, the hairpin-ended DNA molecule comprises an initiation codon GGG. In another preferred embodiment, the hairpin-ended DNA molecule comprises an initiation codon AGA. In some embodiments, the sequence CRCCaugG (R=purine, A or G) may provide optimal context for AUG recognition in eukaryotes.

As disclosed in section 5.1.1(c)(i), in certain embodiments, the transcription unit can comprise a termination signal, a polyadenylation sequence, or both a polyadenylation and termination signal. The polyadenylation sequence may be directly encoded in the transcription unit such that the transcript is directly synthesized with a polyadenylation sequence. Without wishing to be bound by theory, the polyadenylation sequence can prevent degradation of the RNA molecule in a cell. In certain embodiments, the polyadenylation sequence is a homopolymeric sequence comprising an uninterrupted polyA sequence. In certain embodiments, the homopolymeric sequence is between 30-200 nucleotides in length. In some embodiments, the polyadenylation sequence is a homopolymeric sequence comprising polyA sequences interrupted by non-polyA sequences. In some embodiments, the segments of the polyA sequences are between 30-100 nucleotides in length. In some embodiments, the segments of the non-polyA sequences are between 1-15 nucleotides in length. In some embodiments, a polyadenylation sequence is added to the RNA molecule produced by the host cell by enzymatic treatment with a Poly(A) polymerase. In some embodiments, the double-stranded DNA molecule comprises a termination signal capable of signaling the end of transcription. In certain embodiments, the double-stranded DNA molecule comprises a T7 termination signal In some embodiments, the hairpin-ended DNA molecule comprises a recognition site for a restriction enzyme immediately downstream of the encoded polyadenylation sequence and/or the termination signal. In certain embodiments, the method of producing RNA further comprises before step (d), cleaving the hairpin-ended DNA molecule at the downstream restriction site with a restriction enzyme to remove downstream sequences (e.g., the inverted repeat). Without wishing to be bound by theory, removal of the downstream sequences mitigates transcriptional readthrough. In some embodiments, the hairpin-ended DNA molecule further comprises a recognition site for a restriction enzyme upstream of the transcription unit. In some embodiments, the method of producing RNA further comprises before step (d), cleaving the hairpin-ended DNA molecule at the upstream and/or downstream restriction site(s) with one or more restriction enzyme to remove upstream and/or downstream sequences (e.g., the inverted repeats). In some embodiments, the sequence of the upstream and downstream recognition sites are the same.

In some embodiments, the hairpin-ended DNA molecule comprises an expression cassette as described in Section 5.1.1(c)(i) comprising a transcription unit suitable for IVT, wherein the transcription unit comprises an RNA polymerase promoter sequence operably linked to an ORF, a 5′ untranslated region (UTR) and/or a 3′ UTR, a polyadenylation sequence comprising 30-200 nucleotides, and a restriction site. In some embodiments, the hairpin-ended DNA molecule comprises an expression cassette (as described in Section 5.1.1(c)(i)) comprising a transcription unit suitable for IVT, wherein the transcription unit comprises (i) an RNA polymerase promoter sequence operably linked to a nucleotide sequence encoding an RNA polymerase, which is operably linked to a subgenomic promoter, which is operably linked to an ORF; (ii) a 5′ untranslated region (UTR) and/or a 3′ UTR; (iii) a polyadenylation sequence comprising 30-200 nucleotides; (iv) and a restriction site.

In some embodiments, the hairpin-ended DNA molecules may be processed prior to transcribing (e.g. by IVT) in step (d). In certain embodiments, the processing prior to step (d) produces an IVT template. In some embodiments, processing the hairpin-ended DNA molecules comprises digesting the hairpin-ended DNA molecule with a restriction enzyme. In certain embodiments, the digesting is capable of removing DNA sequences downstream of the polyadenylation sequence (e.g. downstream inverted repeat(s)). In some embodiments, processing the hairpin-ended DNA molecules comprises digesting the hairpin-ended DNA molecule with a restriction enzyme to remove downstream and upstream inverted repeats. In certain embodiments, the digesting with the restriction enzyme results in an IVT template comprising blunt ends. In certain embodiments, the digesting with the restriction enzyme results in an IVT template comprising a 3′ overhang. Illustrative restriction enzymes that can be used with the methods and compositions provided herein include Type II enzymes, Type IIS enzymes, Type IIb enzymes, Type Ile enzymes, and enzymes listed in Table 15. In a preferred embodiment, the restriction enzyme is a Type IIS enzyme. In some embodiments, the IVT template is purified before the transcribing in step (d). For example, the IVT template may be purified by PCR cleanup kits, ethanol precipitation, or chromatographically.

In certain embodiments, the transcribing in step (d) comprises contacting the hairpin-ended DNA molecule or IVT template with an in vitro transcription reaction system comprising an RNA polymerase and ribonucleotides. In certain embodiments, the contacting occurs under conditions suitable for in vitro transcription to produce at least one mRNA molecule. In certain embodiments, the hairpin-ended DNA molecule or IVT template are contacted with the in vitro transcription reaction system for between about 120 minutes and about 260 minutes. In certain embodiments, the hairpin-ended DNA molecule or IVT template are contacted with the in vitro transcription reaction system at a temperature between about 25° C. and 45° C. In some embodiments, the hairpin-ended DNA molecule or IVT template are agitated while being contacted with the in vitro transcription reaction system. In certain embodiments, the in vitro transcription reaction system further comprises a buffer, a magnesium ion, and/or a reducing agent. In certain embodiments, the in vitro transcription reaction system further comprises a buffer, a magnesium ion, and a reducing agent. In some embodiments, the buffer is selected from the group consisting of TRIS and HEPES. In some embodiments, the buffer does not contain dithiothreitol (DTT). In certain embodiments the in vitro transcription reaction system lacks a polyamine. In some embodiments, the in vitro transcription reaction system further comprises a ribonuclease inhibitor. In some embodiments, the in vitro transcription reaction further comprises pyrophosphatase. In certain embodiments, the contacting further comprises supplementing the in vitro transcription reaction system with ribonucleotides.

Any RNA polymerase or variants thereof may be used in the methods described here. In certain embodiments, the RNA polymerase is selected from the group consisting of a phage RNA polymerase (e.g., a T7 RNA polymerase, a T3 RNA polymerase, a Syn5 RNA polymerase, or a SP6 RNA polymerase) and mutant polymerases (such as, but not limited to, polymerases capable of incorporating modified nucleic acids, polymerases capable of selectively incorporating cap analogues, or polymerases that are thermostable/thermophilic). In certain embodiments, the RNA polymerase is a T7 RNA polymerase, a T3 RNA polymerase, a Syn5 RNA polymerase, or a SP6 RNA polymerase.

In certain embodiments, the ribonucleotides are selected from the group consisting of any natural ribonucleotides (e.g. A, G, C, and U ribonucleotides) and modified ribonucleotides (such as, for example, N-1-methylpseudouridine triphosphate), or a combination thereof. In some embodiments, the ribonucleotides are Tris buffered. In certain embodiments, the hairpin-ended DNA molecule or IVT template are contacted with an in vitro transcription reaction system comprising a 100 mM aqueous solution of the ribonucleotide, wherein the aqueous solution has been titrated to pH 7.3-7.5 with Tris base. In some embodiments, the ribonucleotides are sodium salts of natural or modified ribonucleotides. Modified nucleobases may be incorporated into modified nucleosides and modified ribonucleotides. In certain embodiments, the in vitro transcription reaction system comprises modified ribonucleotides selected from the group consisting of m5C (5-methylcytidine), m5U (5-methyluridine), m6A (N6-methyladenosine), s2U (2-thiouridine), Um (2′-0-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms216A (2-methylthio-N6isopentenyladenosine); io6A (N6-(cis-hydroxyisopentenyl) adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyl adenosine); ms2hn6A (2-methylthio-N6-hydroxynorvalyl carbamoyladenosine); Ar (p) (2′-0-ribosyladenosine (phosphate)); I (inosine); mil (1-methylinosine); m′lm (1,2′-0-dimethylinosine); m3C (3-methylcytidine); Cm (2T-0-methylcytidine); s2C (2-thiocytidine); ac4C (N4-acetylcytidine); £5C (5-fonnylcytidine); m5Cm (5,2-0-dimethylcytidine); ac4Cm (N4acetyl2TOmethylcytidine); k2C (lysidine); mIG (1-methylguanosine); m2G (N2-methylguanosine); m7G (7-methylguanosine); Gm (2′-0-methylguanosine); m22G (N2,N2-dimethylguanosine); m2Gm (N2,2′-0-dimethylguanosine); m22Gm (N2,N2,2′-0-trimethylguanosine); Gr(p) (2′-0-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-0-dimethyluridine); s4U (4-thiouridine); m5s2U (5-methyl-2-thiouridine); s2Um (2-thio-2′-O-methyluridine); acp3U (3-(3-amino-3-carboxypropyl) uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl) uridine)); mchm5U (5-(carboxyhydroxymethyl) uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-0-methyluridine); cmnm5U (5-carboxymethylaminomethyluridine); cnmm5Um (5-carboxymethy 1 aminomethyl-2-L-Omethyluridine); cmnm5s2U (5-carboxymethylaminomethyl-2-thiouridine); m62A (N6,N6-dimethyladenosine); Tm (2′-0-methylinosine); m4C (N4-methylcytidine); m4Cm (N4,2-0-dimethylcytidine); hm5C (5-hydroxymethylcytidine); m3U (3-methyluridine); cm5U (5-carboxymethyluridine); m6Am (N6,T-0-dimethyladenosine); rn62Am (N6,N6,0-2-trimethyladenosine); m2′7G (N2,7-dimethylguanosine); m2′2′7G (N2,N2,7-trimethylguanosine); m3Um (3,2T-0-dimethyluridine); m5D (5-methyldihydrouridine); f5Cm (5-formyl-2′-0-methylcytidine); mIGm (1,2′-0-dimethylguanosine); m′Am (1,2-0-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(Ci-C6)-alkyluracil, 5-methyluracil, 5-(C2-Ce)-alkenyluracil, 5-(C2-Ce)-alkynyluracil, 5-(hydroxymethyl) uracil, 5-chlorouracil, 5-fluorouracil, 5-bromouracil, 5-hydroxycytosine, 5-(Ci-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, and 2′-0-methyl-U, or combinations thereof. In certain embodiments, the in vitro transcription reaction system comprises modified ribonucleotides selected from the group consisting of N-1-methylpseudouridine; pseudouridine, N6-methyladenosine, 5-methylcytidine, and 5-methyluridine, or combinations thereof. In certain embodiments, the RNA product harvested in step (e) comprises phosphoramidate, phosphorothioate, and/or methylphosphonate linkages. In some embodiments, the RNA product harvested in step (e) does not comprise modified nucleotides (e.g., does not include modified nucleobases, and all of the nucleotides in the RNA molecule are conventional standard ribonucleotides A, U, G and C). In some embodiments, the RNA product harvested in step (e) comprises only natural ribonucleotides (i.e. A, U, G, and C) except for an optional 5′ cap comprising one modified ribonucleotide. In certain embodiments, the 5′ cap comprises 7-methylguanosine. In certain embodiments, the RNA product harvested in step (e) comprises natural ribonucleotides (i.e. A, U, G, and C) that are methylated at the 2′ position of the ribose. In certain embodiments, any or all of the first one, two, or three ribonucleotides on the 5′ end of the RNA product are methylated at the 2′ position of the ribose. In certain embodiments, the RNA product harvested in step (e) comprises only natural ribonucleotides (i.e. A, U, G, and C) except for a 5′ cap comprising a 7′-methylguanosine, and wherein the first one, two, or three ribonucleotides on the 5′ end of the RNA product are methylated at the 2′ position of the ribose.

In some embodiments, the in vitro transcription reaction system (e.g. after mixing with the hairpin-ended DNA molecule) comprises ribonucleotides in an amount of between about 16 and about 50 mM. In some embodiments, the in vitro transcription reaction system comprises the RNA polymerase in an amount of between about 4000 and about 12000 U/mL. In some embodiments, the in vitro transcription reaction system (e.g. after mixing with the hairpin-ended DNA molecule) comprises pyrophosphatase in an amount of between about 0.02 and about 8.0 U/mL. In some embodiments, the in vitro transcription reaction system comprises a buffer having a starting pH between about 7.5 and about 8.5. In some embodiments, the in vitro transcription reaction system comprises magnesium ion in an amount of between about 6 and about 110 mM. In some embodiments, the in vitro transcription reaction system comprises magnesium ion and the ribonucleotides in a magnesium:ribonucleotide ratio of about 0.8 to about 2.2 (mM/mM).

In some embodiments, the transcribing in step (d) comprises co-transcriptional capping. In certain embodiments, the co-transcriptional capping comprises 5′ capping of the RNA product. In some embodiments, the co-transcriptional capping comprises use of Anti-Reverse Cap Analogs (ARCA). In some embodiments, the co-transcriptional capping comprises enzymatic capping of the 5′ end of the RNA product. In some embodiments, the transcribing in step (d) comprises enzymatic polyadenylation of the RNA product after transcription.

In some embodiments, the harvesting in step (e) comprises use of a capture substrate selected from the group consisting of an oligo (dT) magnetic bead, a resin, and a monolith. In some embodiments, the harvesting comprises capturing the RNA product on a capture substrate followed by washing the captured substrate (e.g. the oligo (dT) magnetic bead, the resin, or the monolith) and collecting the RNA product. In some embodiments, the washing comprises contacting the capture substrate with a solution comprising a aqueous buffer selected from the group consisting of Tris buffer, HEPES buffer, NaPi buffer, KCl buffer, NaCl buffer, Urea buffer, Arginine buffer, and EDTA buffer.

In certain embodiments, hairpin-ended DNA molecules provided herein can be used for non-viral gene therapy. Any non-viral DNA delivery system can be used. Such delivery systems include transfection of naked DNA, lipid-based delivery systems, and polymer-based delivery systems. In certain embodiments, hybridosomes can be used as the delivery systems (e.g., hybridosomes disclosed in WO2015/110957, the content of which is incorporated by reference). Other DNA delivery systems that can be used to deliver hairpin-ended DNA molecules as generated by the methods provided herein to cells are described in Sung & Kim. Biomater Res. (2019); 23:8.

### 5.6 Kits

The present disclosure further provides kits for making the presently disclosed hairpin-ended DNA molecules or the intermediate molecules thereof (e.g., amplification products, precursors of the hairpin-ended DNA molecules).

### 5.6.1 Kits for Amplifying DNA Templates

In one aspect, the present disclosure provides a kit comprising means for amplifying (e.g., isothermal amplification, e.g., RCA, MDA) a DNA template (e.g., the DNA templates disclosed in Section 5.1) to make at least one amplification product (e.g., the amplification product disclosed in Section 5.1).

In certain embodiments, the kit comprises a DNA template, a DNA polymerase, and a primer pair. In certain embodiments, the kit further comprises dNTPs and a buffer. In certain embodiments, the kit further comprises a reagent capable of terminating the amplification reaction. In certain embodiments, the kit further comprises an MSRE or an MSNE.

In certain embodiments, the DNA template comprised in the kit disclosed herein is a circular DNA. In certain embodiments, the DNA template is a double-stranded circular DNA. In certain embodiments, the DNA template is a single-stranded circular DNA. In certain embodiments, the DNA template comprises inverted repeats as described in Section 5.1.1(a), nicking endonuclease sites as described in Section 5.1.1(b), and a sequence of interest as described in Section 5.1.1(c). In certain embodiments, the DNA template further comprises a restriction enzyme site as described in 5.1.3 and/or additional nicking endonuclease sites as described in Section 5.1.4. In certain embodiments, the DNA template comprises an MSRE site as described in Section 5.1.3(a), wherein the MSRE is methylated. In certain embodiments, the DNA template comprises an MSNE site as described in Section 5.1.4(a), wherein the MSRE is methylated.

In certain embodiments, the DNA polymerase comprised in the kit disclosed herein is a DNA polymerase as described in Section 5.2.2(a). In certain embodiments, the DNA polymerase is an M2 polymerase.

In certain embodiments, the primers comprised in the kit disclosed herein are primers as described in Section 5.2.2 (b). In certain embodiments, the primers are selected from the group consisting of sequence-specific primers, random primers, and combinations thereof. In certain embodiments, the primers are site specific primers. In certain embodiments, the site specific primers are a primer-pair. Binding sites for sequence-specific primers are described in Section 5.1.2.

In certain embodiments, the kit further comprises dNTPs as described in Section 5.2.2 (c).

In certain embodiments, the kit further comprises a buffer suitable for the amplification reaction (e.g., isothermal amplification, e.g., RCA, MDA). One of skill in the art will appreciate that the optimal amplification reaction buffer can depend on the DNA polymerase, the ratio of primers, the amount of DNA polymerase, primers, and/or dNTPs used in the reaction, or any combination thereof. Non-limiting examples of reaction buffer reagents that can be included in the kits provided herein include reducing agents (e.g., dithiothreitol (DTT), dithioerythritol (DTE), tris(2-carboxyethyl)phosphine (TCEP), β-mercaptoethanol (BME)), pyrophosphatases, metals and/or salts thereof (e.g., Mg, MgCl₂, Mn, MnCl), albumin, and buffering reagents (e.g., Tris-acetate (Tris-OAc), magnesium acetate (Mg(OAc)₂), potassium acetate (CH₃COOK), Tris-HCl).

In certain embodiments, the kit further comprises a reagent capable of terminating the amplification reaction. Reagents capable of terminating the amplification reaction are described in Section 5.2.3. In certain embodiments, the kit comprises a buffer for adjusting pH of the amplification reaction mixture (e.g., phosphate buffer, carbonate buffer, citrate buffer, acetate buffer, Tris hydrochloride buffer). In certain embodiments, the kit comprises a cationic chelating agent (e.g., NTA, EDTA, DMNP-EDTA, HEDP, EDTMPS, DTPMPA, EDDHA, STPP, sodium dextrose, sodium metasilicate).

In certain embodiments, the kit further comprises an MSRE and/or an MSNE. In certain embodiments, the kit comprises an MSRE as described in Section 5.2.4. In certain embodiments, the kit comprises an MSNE as described in Section 5.2.4.

In certain embodiments, the kit further comprises an additional enzyme disclosed in Section 5.2.2 (d). In certain embodiments, the additional enzyme is selected from the group consisting of pyrophosphatase, helicase, SSBs, PrimPol, and combinations thereof.

In certain embodiments, the kit further comprises instruction(s) for performing the methods of amplifying the DNA template as described in Section 5.2.

### 5.6.2 Kits for Making Hairpin-ended DNA Molecules and Precursors Thereof

In one aspect, the present disclosure provides a kit comprising means for making precursors of hairpin-ended DNA molecules. In certain embodiments, the kit comprises at least one amplification product (e.g., amplification products as described in Section 5.1), and at least one of a restriction enzyme, a nicking endonuclease, an MSNE, and an MSRE as described in Section 5.3.1.

In another aspect, the present disclosure provides a kit comprising means for making hairpin-ended DNA molecules. In certain embodiments, the kit comprises at least one amplification product (e.g., amplification products as described in Section 5.1) and a nicking endonuclease as described in Section 5.3.2. In certain embodiments, the kit further comprises a ligase as described in Section 5.3.5. In certain embodiments, the kit further comprises at least one of a restriction enzyme, a nicking endonuclease, an MSNE, and an MSRE as described in Section 5.3.1 for making precursors of hairpin-ended DNA molecules.

In certain embodiments, the amplification product comprises inverted repeats as described in Section 5.1.1(a), nicking endonuclease sites as described in Section 5.1.1(b), and a sequence of interest as described in Section 5.1.1(c). In certain embodiments, the amplification product further comprises a restriction enzyme site as described in 5.1.3 and/or additional nicking endonuclease sites as described in Section 5.1.4. In certain embodiments, the amplification product comprises an MSRE site as described in Section 5.1.3(a), wherein the MSRE site is unmethylated. In certain embodiments, the amplification product comprises an MSNE site as described in Section 5.1.4(a), wherein the MSNE site is unmethylated.

In certain embodiments, kits disclosed herein further comprise reagents for making hairpin-ended DNA molecules having a high purity. In certain embodiments, the kit comprises an exonuclease as described in Section 5.4.2 for digesting undesired DNA molecules. In certain embodiments, the kit further comprises at least one of a restriction enzyme, a nicking endonuclease, an MSNE, and an MSRE as described in Section 5.4.1 for generating double strand breaks for endonuclease digestion.

In certain embodiments, the kit further comprises instruction(s) for making precursors of hairpin-ended DNA molecules as described in Section 5.3.1. In certain embodiments, the kit further comprises instruction(s) for making hairpin-ended DNA molecules as described in Sections 5.3.2, 5.3.3, 5.3.4, and 5.3.5. In certain embodiments, the kit further comprises instruction(s) for removing undesired DNA molecules as described in Section 5.4.

In certain embodiments, kits disclosed in Sections 5.6.1 and 5.6.2 can be combined with each other. In certain embodiments, kits disclosed in Sections 5.6.1 and 5.6.2 can include instructions for practicing any of the methods described herein (e.g., methods toward generating the invention). In certain embodiments, the reagents of the kits disclosed in Sections 5.6.1 and 5.6.2 can be packaged in a reaction container suitable for performing any of the methods described herein. In certain embodiments, a reaction container suitable for packaging the reagents of the kits described herein can include a tube (e.g., a capillary tube), a vial, a capsule, an ampule, or another suitable vessel. Typically, a reaction container can have a removable component and/or a replaceable component wherein said component can seal the reaction container such that the reaction container is water-tight and/or air-tight. In certain embodiments, any of the kits disclosed in Sections 5.6.1 and 5.6.2 can comprise a solid support or matrix to which reagents of the kits can be attached, immobilized, or impregnated within.

### 6. EQUIVALENTS

All patents and publications mentioned in this specification are incorporated herein by reference in their entireties. From the foregoing description, it will be apparent that variations and modifications can be made to the invention described herein to adopt it to various uses and conditions. Such embodiments are also within the scope of the following claims. Although the disclosed subject matter has been described with reference to the examples provided below, it should be understood that various modifications can be made without departing from the spirit of the disclosed subject matter. Many variations will become apparent to those skilled in the art upon review of this specification.

### 7. EXAMPLES

### 7.1 Example 1: Generation of Hairpin-Ended DNA after DNA Amplification with Primer Pairs Binding at Various Locations in the DNA Template

A nucleic acid sequence, encoding an expression cassette flanked by a 3′ ITR and a 5′ ITR was designed in silico and cloned into a pUC57 backbone. The final ~8 kilobase (kb) circular template DNA was transformed and amplified overnight in a NEB stable Escherichia coli strain followed by plasmid isolation using a commercial plasmid isolation kit (Nucleobond Xtra Maxi Plus EF (Macherey Nagel)). The isolated plasmid was dissolved in 5 mM TRIS, 0.1 mM EDTA, pH 8.0.

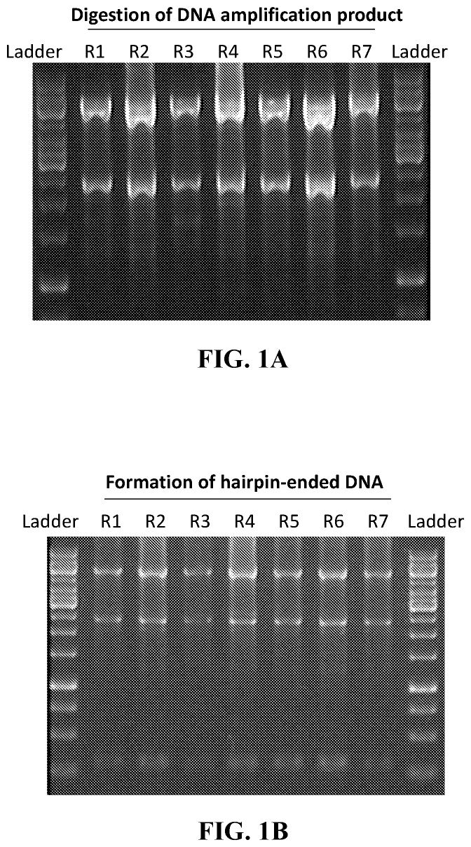

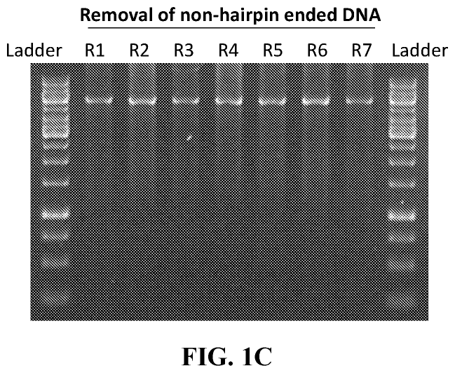

Each amplification reaction (R1-R7) was prepared by annealing the respective primer pairs listed in Table 22 to the circular DNA template prepared above. Each amplification reaction had a final concentration of 2 ng/μL circular DNA template, 1 μM forward primer, and 1 μM reverse primer. Primer annealing was performed by mixing DNAse-free water, the circular DNA template, and the primer pair together before adding 1 M NaOH to increase the pH to ~pH 12. The resulting mixture was neutralized with 10× amplification buffer (500 mM TRIS, 300 mM KCl, pH 8.0) to yield a 1× amplification buffer in the final amplification reaction volume. Further, a dNTP mixture (dATP, dCTP, dTTP, dGTP), MgCl₂, and recombinant inorganic pyrophosphatase were added to the reaction to bring their final concentration to 15 mM, 15 mM, and 1500 ng/mL, respectively. The amplification reaction was initialized by adding recombinant M2-phage DNA polymerase (0.03 mg/mL). DNA amplification was performed for 5.5 hours at 30° C. and 800 rpm orbital shaking in a 1.5 mL polypropylene tube. The amplification reactions were terminated by: diluting the reaction 1:1 with nicking reaction buffer (300 mM TRIS, 270 mM NaCl, 10 mM MgCl₂, pH 9.0); adding recombinant Nt.BspQI (0.015 mg/mL); and incubating for 2 hours at 37° C. under orbital shaking with 800 rpm. Quenching of the reaction was performed by adding an equimolar ratio of EDTA to magnesium (EDTA:Mg) present in the reaction. Digestion of the reactions (R1-R7) was confirmed by agarose gel analysis (FIG. 1A). The gel showed two main bands resulting from the two antiparallel double nicking sites adjacent to the 3′ ITR and 5′ ITR in the backbone sequence of the amplified DNA. The upper band (around 6 kB) fragment also contained additional nicks to allow for later formation of hairpin-ended DNA.

TABLE 22
Primer Pairs for Amplification Reactions R1-R7

| | | | Distance of | Direction of |
|---|---|---|---|---|
| | | | primer | isothermal |
| | | Primer sequence | annealing sites | amplification |
| | | *marks phosphorothioate (PTO) | in template | from |
| Reaction | Primer | modification) | backbone | each primer |
| R1 | Forward | 5′-GAAACCCGACAG*G*A*C-3′ | 0 bp | Away from each other |
| | primer | (SEQ ID NO: 511) | | |
| | Reverse | 5′-AGAATGACTTGG*T*T*G-3′ | | |
| | primer | (SEQ ID NO: 512) | | |
| R2 | Forward | 5′-AGATCCAGTTCD*A*T*G-3′ | ~100 bp | Towards each other |
| | primer | (SEQ ID NO: 513) | | |
| | Reverse | 5′-GTGTCGCCCTTA*T*T*C-3′ | | |
| | primer | (SEQ ID NO: 514) | | |
| R3 | Forward | 5′-GCTACAGAGTTC*T*T*G-3′ | ~1,000 bp | Towards each other |
| | primer | (SEQ ID NO: 515) | | |
| | Reverse | 5′-AGAATGACTTGG*T*T*G-3′ | | |
| | primer | (SEQ ID NO: 516) | | |
| R4 | Forward | 5′-GAAACCCGACAG*G*A*C-3′ | ~1,500 bp | Towards each other |
| | primer | (SEQ ID NO: 517) | | |
| | Reverse | 5′-GTGTCGCCCTTA*T*T*C-3′ | | |
| | primer | (SEQ ID NO: 518) | | |
| R5 | Forward | 5′-GAATAAGGGCGA*C*A*C-3′ | ~1,500 bp | Away from each other |
| | primer | (SEQ ID NO: 519) | | |
| | Reverse | 5′-GTCCTGTCGGGT*T*T*C-3′ | | |
| | primer | (SEQ ID NO: 520) | | |
| R6 | Forward | 5′-CAACCAAGTCAT*T*C*T-3′ | ~1,000 bp | Away from each other |
| | primer | (SEQ ID NO: 521) | | |
| | Reverse | 5′-CAAGAACTCTGT*A*G*C-3′ | | |
| | primer | (SEQ ID NO: 522) | | |
| R7 | Forward | 5′-AGATCCAGTTCG*A*T*G-3′ | ~150 bp | Away from each other |
| | primer | (SEQ ID NO: 523) | | |
| | Reverse | 5′-AGAATGACTTGG*T*T*G-3′ | | |
| | primer | (SEQ ID NO: 524) | | |

Nt.BspQI-digested amplified DNA products were then used for formation of hairpin-ended DNA constructs. Strand separation was performed by increasing the pH with NaOH to around pH 12, followed by neutralization by addition of TRIS, pH 6.9 to bring the pH below pH 9. Reduction of pH resulted in re-annealing of the separated DNA strands, but with formation of hairpin ends due to intramolecular forces. FIG. 1B shows formation of hairpin ends, appearing as low molecular DNA fragments (~200-300 bp) that are displaced through intramolecular forces that drive ITR formation.

Exonuclease digestion was then performed by diluting the mixture containing the hairpin-ended DNA with 1× digestion buffer (20 mM TRIS, 100 mM NaCl, 10 mM MgCl₂, pH 8.4) and adding a truncated exonuclease VIII to a final DNA to exonuclease ratio of 20:1-40:1. Removal of non-hairpin-ended DNA and resistance of hairpin-ended DNA to exonuclease digestion was checked by agarose gel after 3 hours at 37° C. and 800 rpm orbital shaking. As shown in FIG. 1C, only one clear band, being the hairpin-ended DNA, remained after exonuclease digest. Quenching of the reactions was performed by adding EDTA in an equimolar ratio to magnesium in the reactions in order to the chelate the magnesium.

FIGS. 1A-1C demonstrate that amplification and generation of hairpin-ended DNA molecules is feasible when different primer pairs having varying binding loci in the template are used. FIGS. 1A-1C show 1% agarose gels run in 1×TAE buffer (40 mM TRIS, 40 mM acetate, 1 mM EDTA, PH~8.3). FIG. 1A shows an agarose gel of a reaction sample after the nicking of the amplified DNA product. FIG. 1B shows an agarose gel of a reaction sample after the formation of hairpin-ended DNA through processing of the amplification digestion product. FIG. 1C shows an agarose gel demonstrating the removal of non-hairpin ended DNA by an exonuclease.

### 7.2 Example 2: Impact of Mg:dNTP:KCl Ratio and its Impact on Final DNA Yield and Amplification Factor of Amplification Reactions

The ratio of Mg:dNTP:KCl affected the reaction rate of the DNA amplification. Therefore, fine-tuning the ratio of Mg:dNTP:KCl was used to increase the reaction rate of amplification reactions.

Amplification reactions, termination of reactions, digestion of amplification products and quenching of digestions by EDTA were performed according to the methods described in Example 1. However, in this example, the isothermal amplification was performed for 14 hours at 30° C. with M2-phage DNA polymerase in reactions containing 30 mM dNTP mix (dATP, dCTP, dGTP, dTTP) and varying concentrations of MgCl₂.

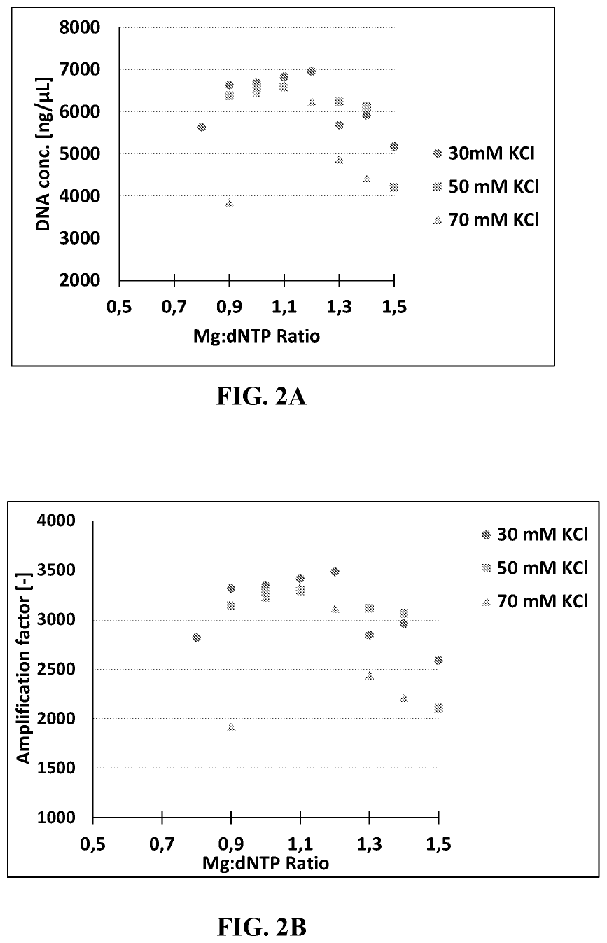

After digestion, reactions were quenched using a reverse phase ion-pairing chromatography column according to HPLC methods known in the art to separate the DNA from protein, unused primers, and/or unused dNTPs. DNA concentrations were measured after digestion and quenching (FIG. 2A). The amplification reaction yielded a DNA synthesis rate around 460 ng/μL per hour at a Mg:dNTP ratio between 1.0-1.2 and a KCl concentration between 30-70 mM. Amplification factors were calculated by taking an initial starting concentration of 2 ng/μL template into account. For example, starting with a template amount of 2 ng/μL in an amplification reaction results in around 6000 ng/μL amplified DNA with an amplification factor of 3000. Amplification factors around 3500 were achieved as shown in FIG. 2B.

### 7.3 Example 3: Transfection of Amplified Hairpin Ended DNA

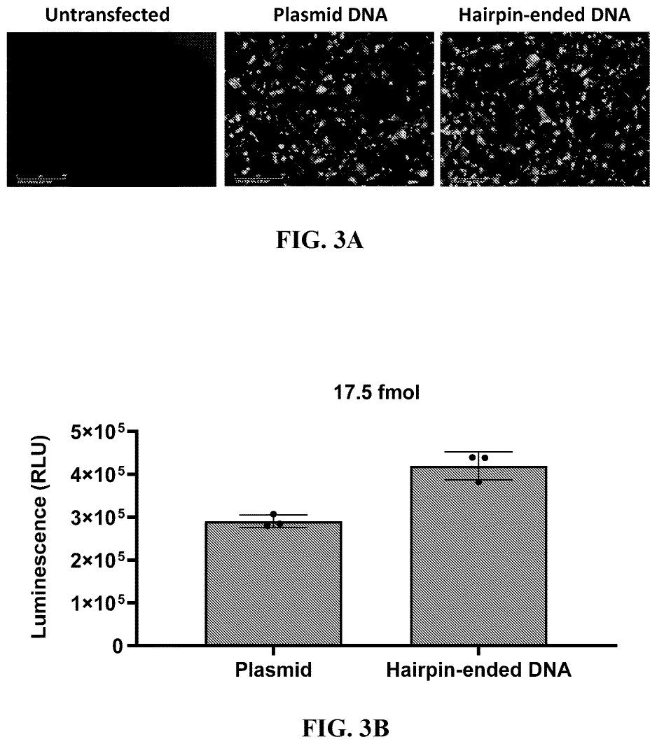

Huh-7 cells were seeded in 96-well plates at a density of 20,000 cells/well in standard growth media supplemented with 10% fetal bovine serum and left to adhere overnight. After 24 hours, media was changed and cells were transfected with amplified hairpin-ended DNA (prepared as described in Example 1) or its corresponding plasmid DNA template encoding firefly luciferase (fLuc)-T2A-enhanced green fluorescent protein (EGFP). Amplified hairpin-ended DNA or plasmid DNA template were formulated in Lipofectamine 3000 (ThermoFisher Scientific; Cat No. L3000008) following the manufacturer's protocol and the resulting transfection mixture was added to the cells, in triplicates, at a concentration of 17.5 fmol. At 48 hours post-transfection, cells were imaged on Incucyte SX5 for EGFP detection (FIG. 3A). Firefly luciferase activity was determined using the Bio-Glo Luciferase assay (Promega; Cat No. G7941). In brief, cell culture media was removed, and cells were briefly preserved in Dulbecco's phosphate-buffered saline (DPBS) supplemented with 1 mM MgCl₂, followed by the addition of Bio-Glo reagent at a ratio of 1:1 (vol/vol). Cells were incubated for 5 minutes while shaking, protected from light, and luminescence was then measured using a Biotek Cytation 5 plate reader. Relative luminescence units (RLU) were corrected for the background RLU levels of untransfected cells (FIG. 3B). FIGS. 3A and 3B demonstrate that amplified hairpin-ended DNA is readily used for transfection and is functional upon delivery in vitro.

### 7.4 Example 4: Generation of Amplified Hairpin-Ended DNA for mRNA Production from IVT

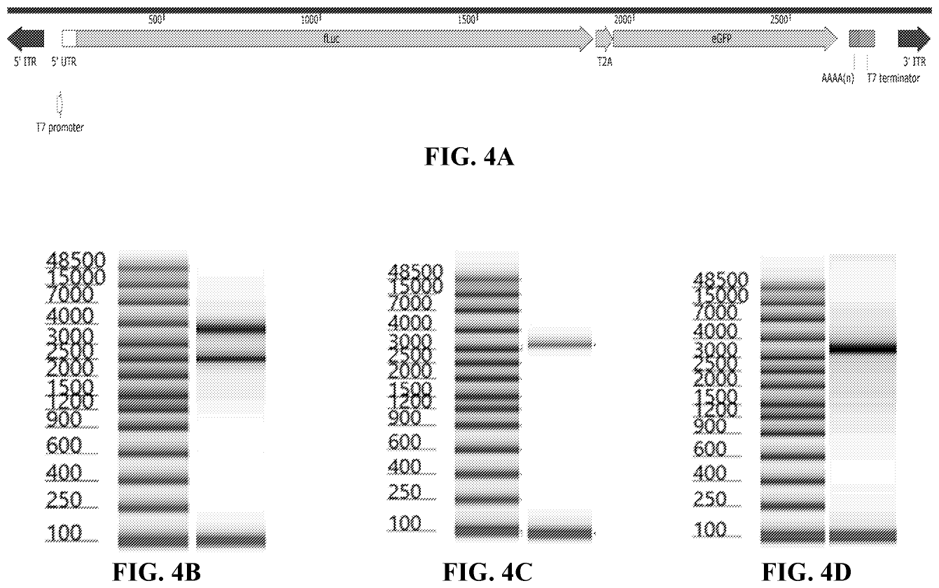

A plasmid DNA template, comprising between the first and fourth restriction sites for nicking endonuclease and in the 5′ to 3′ direction of the top strand: a 5′ ITR; a T7 promoter; a 5′ UTR, a gene codifying firefly luciferase (fLuc), T2A self-cleaving peptide, and eGFP; a spacer; a codifying polyA; a T7 terminator; a spacer; and a 3′ ITR (FIG. 4A and Table 23), was prepared as starting material for the generation of transcription ready hairpin-ended DNA. The initial DNA amplification was performed according to the methods described in Example 1 but with a circular DNA template of around 6 kB. Digestion of the amplification product (FIG. 4B), formation of hairpin-ended DNA, and subsequent exonuclease digest of non-hairpin-ended DNA (FIG. 4C) were performed comparably to that described in Example 1. Finally, amplified hairpin-ended DNA was purified by using a commercially available plasmid purification kit (Qiagen Plasmid Midi Kit) (FIG. 4D).

TABLE 23
Sequence of Hairpin-ended DNA for mRNA Production

| SEQ ID NO | Sequence |
|---|---|
| SEQ ID | AGCAGAAGAGCTCTCTGTACACTCACTCACTCACTGATCCCTGGATACCAAAGGTATCCAGACA |
| NO: 525 | CCCAGTCTTTGACTGGGTGGGATCAGTGAGTGAGTGAGTGTACAGAGAGCTCTTCTGGAGGGAG |
| | TGGCCAAAACCAACTAGACAACTTTGTATACTAATAATACGACTCACTATAGGGAAATAAGAGA |
| | GAAAAGAAGAGTAAGAAGAAATATAAGAGCCACCATGGAAGATGCCAAAAACATTAAGAAGGGC |
| | CCAGCGCCATTCTACCCACTCGAAGACGGGACCGCCGGCGAGCAGCTCCACAAAGCCATGAAGC |
| | GCTACGCCCTGGTGCCCGGCACCATCGCCTTTACCGACGCACATATCGAGGTGGACATTACCTA |
| | CGCCGAGTACTTCGAGATGAGCGTTCGGCTGGCAGAAGCTATGAAGCGCTATGGGCTGAATACA |
| | AACCATCGGATCGTGGTGTGCAGCGAGAATAGCTTGCAGTTCTTCATGCCCGTGTTGGGTGCCC |
| | TGTTCATCGGTGTGGCTGTGGCCCCAGCTAACGACATCTACAACGAGCGCGAGCTGCTGAACAG |
| | CATGGGCATCAGCCAGCCCACCGTCGTATTCGTGAGCAAGAAAGGGCTGCAAAAGATCCTCAAC |
| | GTGCAAAAGAAGCTACCGATCATACAAAAGATCATCATCATGGATAGCAAGACCGACTACCAGG |
| | GCTTCCAAAGCATGTACACCTTCGTGACTTCCCATTTGCCACCCGGCTTCAACGAGTACGACTT |
| | CGTGCCCGAGAGCTTCGACCGGGACAAAACCATCGCCCTGATCATGAACAGTAGTGGCAGTACC |
| | GGATTGCCCAAGGGCGTAGCCCTACCGCACCGCACCGCTTGTGTCCGATTCAGTCATGCCCGCG |
| | ACCCCATCTTCGGCAACCAGATCATCCCCGACACCGCTATCCTGAGCGTGGTGCCATTTCACCA |
| | CGGCTTCGGCATGTTCACCACGCTGGGCTACTTGATCTGCGGCTTTCGGGTCGTGCTCATGTAC |
| | CGCTTCGAGGAGGAGCTATTCTTGCGCAGCTTGCAAGACTATAAGATTCAATCTGCCCTGCTGG |
| | TGCCCACACTATTTAGCTTCTTCGCTAAGAGCACTCTCATCGACAAGTACGACCTAAGCAACTT |
| | GCACGAGATCGCCAGCGGCGGGGCGCCGCTCAGCAAGGAGGTAGGTGAGGCCGTGGCCAAACGC |
| | TTCCACCTACCAGGCATCCGCCAGGGCTACGGCCTGACAGAAACAACCAGCGCCATTCTGATCA |
| | CCCCCGAAGGGGACGACAAGCCTGGCGCAGTAGGCAAGGTGGTGCCCTTCTTCGAGGCTAAGGT |
| | GGTGGACTTGGACACCGGTAAGACACTGGGTGTGAACCAGCGCGGCGAGCTGTGCGTCCGTGGC |
| | CCCATGATCATGAGCGGCTACGTTAACAACCCCGAGGCTACAAACGCTCTCATCGACAAGGACG |
| | GCTGGCTGCACAGCGGCGACATCGCCTACTGGGACGAGGACGAGCACTTCTTCATCGTGGACCG |
| | GCTGAAGTCTCTGATCAAATACAAGGGCTACCAGGTAGCCCCAGCCGAACTGGAGAGCATCCTG |
| | CTGCAACACCCCAACATCTTCGACGCCGGGGTCGCCGGCCTGCCCGACGACGATGCCGGCGAGC |
| | TGCCCGCCGCAGTCGTCGTGCTGGAACACGGTAAAACCATGACCGAGAAGGAGATCGTGGACTA |
| | TGTGGCCAGCCAGGTTACAACCGCCAAGAAGCTGCGCGGTGGTGTTGTGTTCGTGGACGAGGTG |
| | CCTAAAGGACTGACCGGCAAGTTGGACGCCCGCAAGATCCGCGAGATTCTCATTAAGGCCAAGA |
| | AGGGCGGCAAGATCGCCGTGGGATCCGGAGAGGGCAGAGGAAGTCTTCTAACATGCGGTGACGT |
| | GGAGGAGAATCCCGGCCCTATGGTGAGCAAGGGCGAGGAGCTGTTCACCGGGGTGGTGCCCATC |
| | CTGGTCGAGCTGGACGGCGACGTAAACGGCCACAAGTTCAGCGTGTCCGGCGAGGGCGAGGGCG |
| | ATGCCACCTACGGCAAGCTGACCCTGAAGTTCATCTGCACCACCGGCAAGCTGCCCGTGCCCTG |
| | GCCCACCCTCGTGACCACCCTGACCTACGGCGTGCAGTGCTTCAGCCGCTACCCCGACCACATG |
| | AAGCAGCACGACTTCTTCAAGTCCGCCATGCCCGAAGGCTACGTCCAGGAGCGCACCATCTTCT |
| | TCAAGGACGACGGCAACTACAAGACCCGCGCCGAGGTGAAGTTCGAGGGCGACACCCTGGTGAA |
| | CCGCATCGAGCTGAAGGGCATCGACTTCAAGGAGGACGGCAACATCCTGGGGCACAAGCTGGAG |
| | TACAACTACAACAGCCACAACGTCTATATCATGGCCGACAAGCAGAAGAACGGCATCAAGGTGA |
| | ACTTCAAGATCCGCCACAACATCGAGGACGGCAGCGTGCAGCTCGCCGACCACTACCAGCAGAA |
| | CACCCCCATCGGCGACGGCCCCGTGCTGCTGCCCGACAACCACTACCTGAGCACCCAGTCCAAG |
| | CTGAGCAAAGACCCCAACGAGAAGCGCGATCACATGGTCCTGCTGGAGTTCGTGACCGCCGCCG |
| | GGATCACTCTCGGCATGGACGAGCTGTACAAGTAGTGAGGATCCCCACAAAGTGACCTAGAGCT |
| | GGCAGCAAAAAAAAAAAAAAAAAAAAAAAAAAAAAAAACTAGCATAACCCCTTGGGGCCTCTAA |
| | ACGGGTCTTGAGGGGTTTTTTGAACTTCAGGAACCCCTTTTAAGAGTAACATAACTGCTGGAGT |
| | AGATGAAGATGGAGGGCCACTTTACTGGCTCTTCTTCTCTGTACACTCACTCACTCACTGATCC |
| | CACCCAGTCAAAGACTGGGTGTCTGGATACCTTTGGTATCCAGGGATCAGTGAGTGAGTGAGTG |
| | TACAGAGAAGAAGAGC |

### 7.5 Example 5: In Vitro Transcription of Hairpin-Ended DNA Molecules

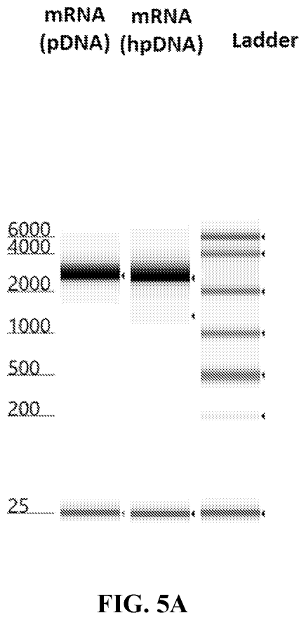

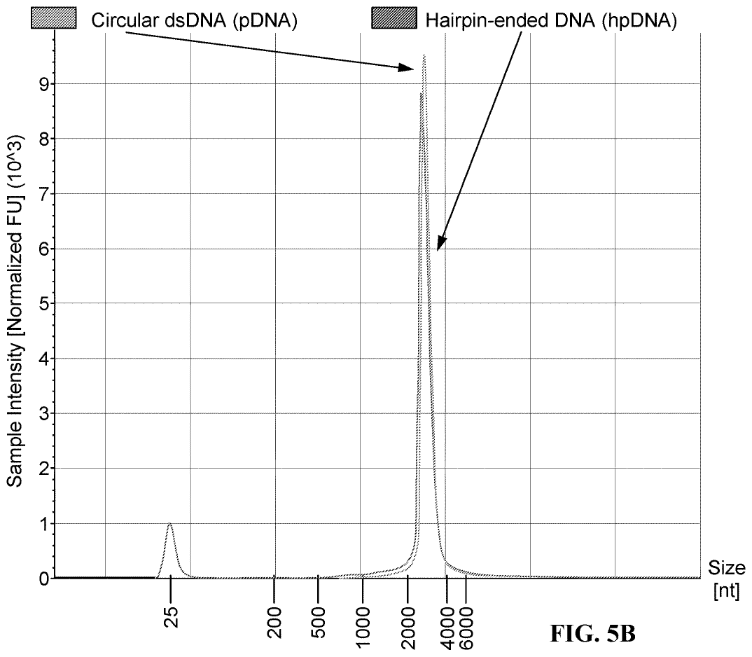

Five (5) μg of amplified hairpin-ended DNA molecules (from Example 4) and the corresponding plasmid DNA template were linearized by adding 1 μl of the BspQI restriction enzyme (NEB; Cat No. R0712S) and 5 μl of NEB 3.1 buffer 10× in water for a final volume of 50 μl. Gel purification was performed from a 1% agarose gel to isolate the desired-size DNA fragment using the NucleoSpin Gel and PCR Clean-up kit from Takara (Cat No. 740609.50). An IVT reaction mixture was prepared for each DNA sample by combining 27 μmol of DNA, 10 μl of 2×ARCA/NTP Mix, 1 μl of RNasin Ribonuclease Inhibitor (Promega; Cat No. N251A), 2 μl of T7 RNA polymerase mix (HiScribe™ T7 ARCA mRNA Kit; Cat No. E2060S), and nuclease free water up to final volume of 20 μl. The reaction was incubated at 37° C. for 40 minutes. Then, 4 μl of DNAse (T5 Exonuclease; Cat No. M0663L) was added and incubated at 37° C. for 15 minutes. Finally, for the IVT reaction, 5 μl of PolyA Polymerase buffer 10× and 5 μl of the PolyA polymerase were added to nuclease free water for a final volume of 50 μl. The reaction was incubated at 37° C. for 30 minutes. Next, mRNA purification was performed with Dynabeads Oligo (dT) 25 (ThermoFisher Scientific; Cat No. 61002) following the manufacturer's protocol. Quality and quantification of the final mRNA produced from linearized plasmid DNA (pDNA) and amplified hairpin ended DNA (hpDNA) templates were analyzed using an Agilent 4200 TapeStation System and an RNA Assay (Cat Nos. 5067-5576, 5067-5577) following the manufacturer's instructions (FIGS. 5A-5B).

### 7.6 Example 6: Transfection of IVT mRNA

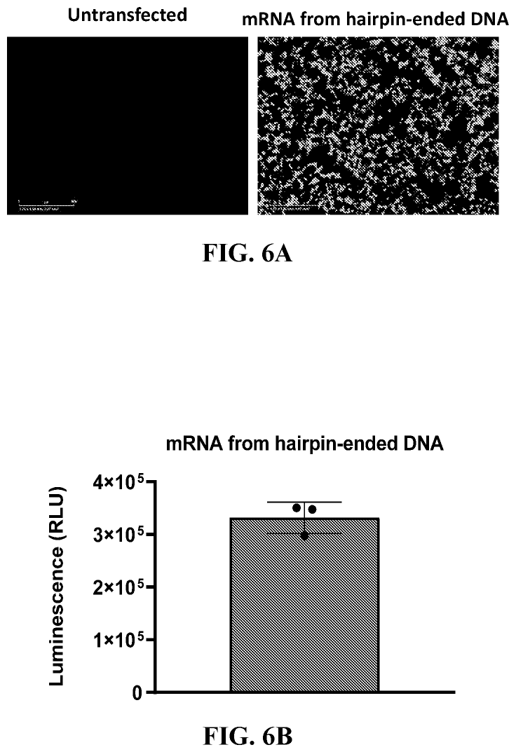

HEK293T cells were seeded in 96-well plates at a density of 20,000 cells/well in standard growth media supplemented with 10% fetal bovine serum and left to adhere overnight. After 24 hours, media was exchanged, and cells were transfected with the amplified hairpin-ended-derived mRNA from Example 5. The mRNA was formulated in Lipofectamine MessengerMAX (ThermoFisher Scientific, Cat No. LMRNA001) following the manufacturer's protocol and the resulting transfection mix was added to the cells, in triplicates, at concentrations of 58.83 ng. After 24 hours post-transfection, cells were imaged on an Incucyte SX5 for EGFP detection (FIG. 6A). Firefly luciferase activity was determined using the Bio-Glo Luciferase assay (Promega; Cat No. G7941) as described herein (FIG. 6B). FIGS. 6A and 6B demonstrate that mRNA synthesized using an amplified hairpin-ended DNA template is readily used for transfection and is functional upon delivery in vitro.

### 7.7 Example 7: Exonuclease Digestion

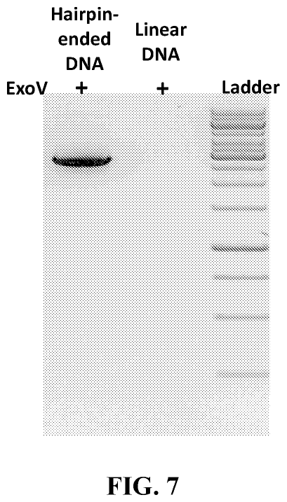

Amplified hairpin-ended DNA, produced according to the methods described in Example 1, was tested for resistance to exonuclease V digestion compared to linearized plasmid. Five hundred (500) ng of hairpin-ended DNA molecules or linearized plasmid molecules were added to a reaction mixture (50 μl total volume) with 1.5 μl of Exonuclease V (NEB; Cat No. M0345S), 5 μl of 10 mM ATP (NEB; Cat No. P0756S), and 4 μl of NEB 4 buffer 10×. The reactions were incubated for 90 minutes at 37° C. The reactions were inactivated at 70° C. for 30 minutes. Ten (10) μl of each reaction was loaded into an 1% agarose gel together with 2 μl of NEB's Gel Loading Dye, Purple (6×) (NEB; Cat No. B7024S) (FIG. 7).

### 7.8 Example 8: Illumina NGS of Hairpin-Ended DNA Molecules

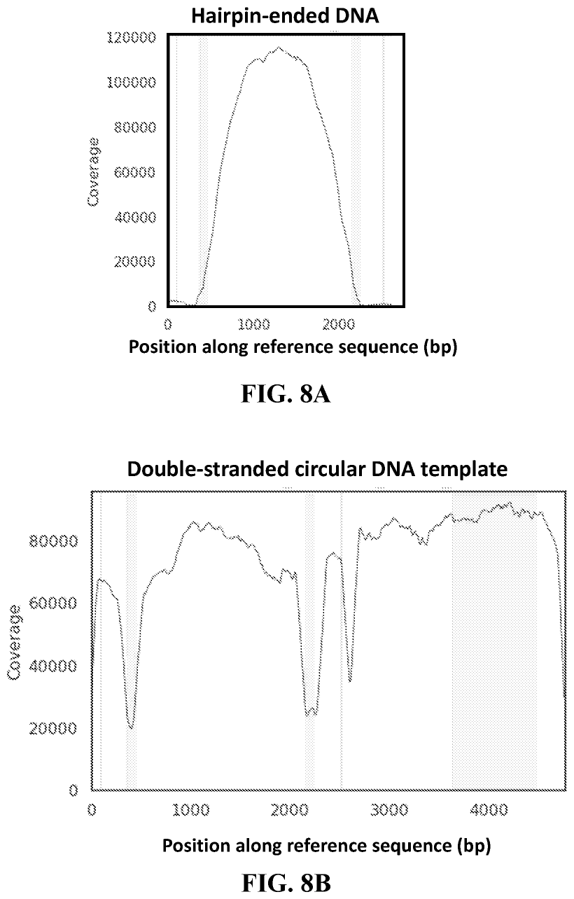

The nicks in the amplified hairpin-ended DNA molecules were repaired by adding ~7 μg of DNA, 2 μl of NEB Hi-T4 DNA ligase, and 5 μl of 10× T4 DNA ligase buffer to a final volume of 50 μl. The reaction mix was incubated for 120 minutes at 25° C. To remove the remaining nicked hairpin-ended DNA molecules, 6 μl of NEBuffer 4 and 2 μl of T5 Exonuclease were added to the reaction which was then incubated for 60 minutes at 37° C. The sample was cleaned up using the NucleoSpin Gel and PCR Clean-up kit from Takara (Cat No. 740609.50). Approximately 0.5 μg of the nick-repaired hairpin-ended DNA and its corresponding unmodified plasmid DNA template were fragmented by mechanical shearing to obtain fragments ~700 bp long. NGS adapters were ligated to the fragments. Sequencing was done on an Illumina NovaSeq (S1 flow cell, 2×100 bp PE read format) generating 1 gigabase of sequencing data (+/−20%) per sample. The sequencing reads were demultiplexed and Illumina adapters were trimmed. Sequencing reads were aligned to the reference sequence of the double-stranded circular DNA template (FIG. 8B) or the hairpin-ended DNA (FIG. 8A). Duplicate reads and those with poor quality or mapping scores were filtered out.

### 7.9 Example 9: Digestions with MSRE

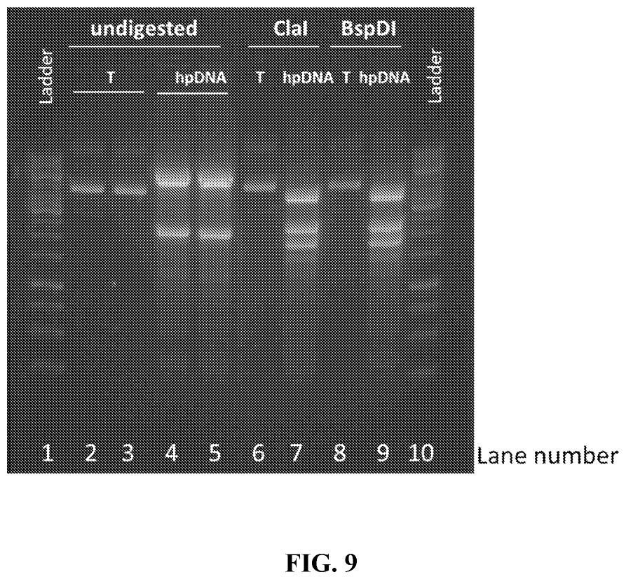

Amplification-product selective digestion with MSREs ClaI and BspDI was assessed. In brief, precursor hairpin-ended DNA (hpDNA) was first prepared by amplifying, nicking, and de/renaturing as described in Example 1 to form the hairpin ended DNA; however, the exonuclease cleanup step of Example 1 was omitted, leaving the backbone intact. Either 500 ng of hpDNA or 500 ng of dam-methylated plasmid DNA template isolated from E. coli were added to a digestion reaction containing ClaI and BspDI (5-10 units of enzyme per μg DNA). The final volume for each digestion reaction was 15 μl. The digestion reactions were incubated at an optimum temperature for enzyme activity as indicated by NEB's protocol (~37° C.) for 1 hour in a thermocycler (VWR XT-96). Then, 3 μl of NEB's Gel Loading Dye, Purple (6×) (Cat No. B7024S) were added to each digestion reaction and all 18 μl were loaded into a 1% agarose gel (FIG. 9). As a control, 500 ng of undigested plasmid DNA template and 500 ng of undigested hpDNA were also loaded into the agarose gel. FIG. 9 shows that only ClaI and BspDI were able to digest the unmethylated hpDNA leaving the methylated double-stranded circular DNA template intact.

### 7.10 Example 10: Using MSRE During Amplification

The nucleic acid sequence, encoding an expression cassette flanked by a 3′ ITR and 5′ ITR is designed in silico and cloned into a pUC57 backbone having specific sites that can be methylated upon production in E. coli NEB stable cells. The final ~6 kB circular template DNA is transformed and amplified overnight in the NEB stable E. coli strain. The plasmid is then isolated using a commercial plasmid isolation kit (Nucleobond Xtra Maxi Plus EF (Macherey Nagel)) and dissolved in 5 mM TRIS, 0.1 mM EDTA, pH 8.0.

Each amplification reaction (R1-R10) is prepared by annealing 25 nucleotide (nt) long 3′ PTO-protected forward and reverse primers to the circular DNA template. Each amplification reaction has a final concentration of 1 ng/μl of plasmid, 1 μM of forward primer, and 1 μM of reverse primer. Primer annealing is performed by mixing DNAse-free water, DNA template, primer pair, and adding 1 M NaOH to increase the pH to ~pH 12 followed by neutralization with 10× amplification buffer (500 mM TRIS, 300 mM KCl, pH 8.0) to yield 1× amplification buffer in the final amplification reaction volume. Further, 10 mM of a dNTP mixture (dATP, dCTP, dTTP, dGTP), 10 mM of MgCl₂, and 3000 ng/ml of recombinant inorganic pyrophosphatase is added to the amplification reaction. The amplification reaction is initialized by adding recombinant M2-phage DNA polymerase (0.015 mg/ml) to reactions R1-R5 and recombinant Phi29-phage DNA polymerase to reactions R6-R10. Five (5) minutes after the addition of the respective DNA polymerases, BspDI, a MSRE, is added to the reactions R1-R4 and R6-R9. Reactions R5 and R10 do not contain BspDI. Reactions R1-R4, and equally, reactions R6-R9 contain different DNA polymerase:BspDI ratios (mg/mL:mg/mL) of 1:1 (R1, R6), 1:0.1(R2, R7), 1:0.05 (R3, R8), and 1:0.01 (R4, R9) to show the impact on final viscosity, DNA yield, and fidelity of the amplification reaction. The reactions are run for 16 hours at 30° C. under 800 rpm orbital shaking in a 1.5 mL polypropylene tube. After 16 hours, a sample is taken of each reaction and the dynamic viscosity is measured at 30° C. with a LOVIS 2000 M microviscometer (Anton Paar). The amplification reactions are then terminated by diluting the reaction 1:1 with nicking reaction buffer (300 mM TRIS, 270 mM NaCl, 10 mM MgCl₂, pH 9.0) and adding recombinant Nt.BspQI (0.015 mg/mL). The reaction is then incubated for 2 hours at 37° C. under orbital shaking at 800 rpm. Quenching of the reaction is performed by adding EDTA in an equimolar ratio to the magnesium present in the reaction. Digestion of the reactions (R1-R10) is confirmed by agarose gel analysis and DNA yields are determined by HPLC using an ion-pairing reverse phase chromatography column. The digested reactions are then prepared for next-generation sequencing to investigate polymerase derived error rates of the amplification reaction products.

### 7.11 Example 11: Generation of Amplified Hairpin-Ended DNA for AAV Production

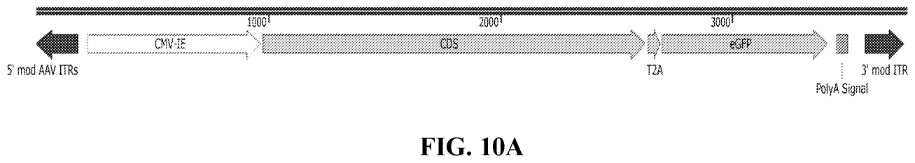

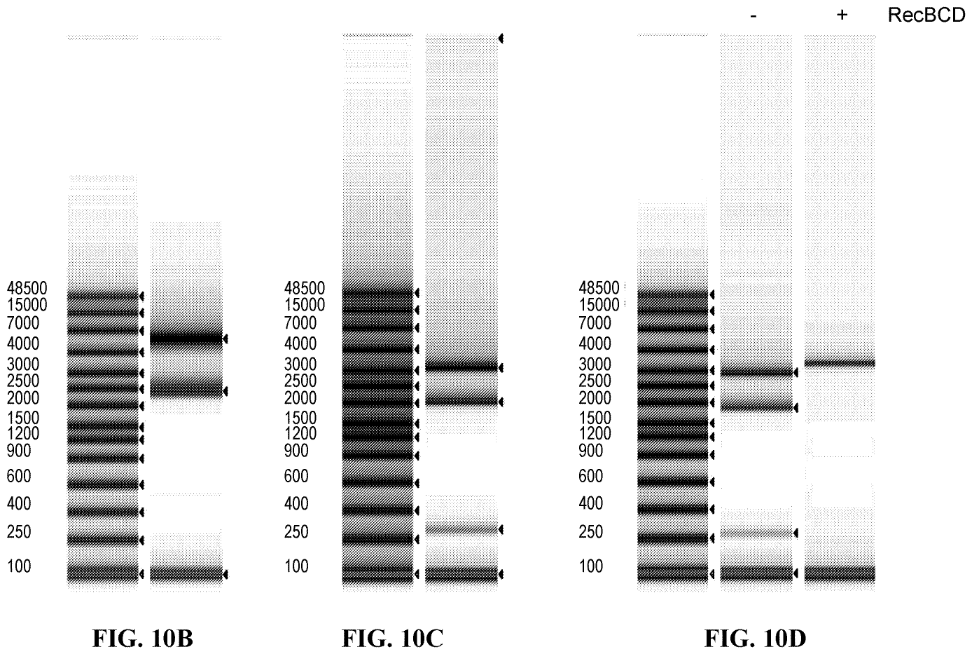

A plasmid DNA template, comprising between the first and fourth restriction sites for nicking endonuclease and in the 5′ to 3′ direction of the top strand: a 5′ ITR derived from AAV2 ITR; a CMV-IE promoter; a 5′ UTR; a gene codifying firefly luciferase (fLuc), T2A self-cleaving peptide, and eGFP; a spacer; a polyA signal; a spacer; and a 3′ ITR derived from AAV2 ITR (FIG. 10A and Table 24) was prepared as starting material for generation of an amplified hairpin-ended DNA that can be used for AAV production. The initial DNA amplification was performed as described in Example 1 but with a circular DNA template of around 6 kB. Digestion of the amplification product (FIG. 10B), formation of hairpin-ended DNA (FIG. 10C), and subsequent exonuclease digest of non-hairpin-ended DNA (FIG. 10D) were performed according to the methods described in Example 1.

Recombinant AAV5 encoding CMV-fLuc-T2A-eGFP is produced as follows. A suspension HEK293 cell line is expanded in a shake flask. After sufficient cell numbers are reached, the production vessel is inoculated. Then, cells are subjected to a triple transfection system which is comprised of amplified hairpin-ended DNA, pRep2Cap5 plasmid, and pHelper plasmid in a 1:1:1 ratio. After 48-72 hours of viral production, the cells are lysed by a chemical reaction. The AAVs in the crude cell lysate are iodixanol-gradient purified and further purified by an ion-exchange chromatography and buffer exchange. Viral genome titer is calculated by TaqMan qPCR and the viral capsid titers are determined by ELISA.

TABLE 24
Sequence of Hairpin-ended DNA for AAV Production

| SEQ ID NO | Sequence |
|---|---|
| SEQ ID | AGAAGAGCAGGAACCCCTAGTGATGGAGTTGGCCACTCCCTCTCTGCGCGCTCGCTCGCTCACT |
| NO: 526 | GAGGCCGGGCGACCAAAGGTCGCCCGACGCCCGGGCTTTGCCCGGGCGGCCTCAGTGAGCGAGC |
| | GAGCGCGCAGAGAGGGAGTGGCCAACTCCATCACTAGGGGTTCCTGCTCTTCTGGAGGGAGTGG |
| | CCAAAACCAACTAGACAACTTTGTATATGAATCAATATTGGCCATTAGCCATATTATTCATTGG |
| | TTATATAGCATAAATCAATATTGGCTATTGGCCATTGCATACGTTGTATCTATATCATAATATG |
| | TACATTTATATTGGCTCATGTCCAATATGACCGCCATGTTGGCATTGATTATTGACTAGTTATT |
| | AATAGTAATCAATTACGGGGTCATTAGTTCATAGCCCATATATGGAGTTCCGCGTTACATAACT |
| | TACGGTAAATGGCCCGCCTGGCTGACCGCCCAACGACCCCCGCCCATTGACGTCAATAATGACG |
| | TATGTTCCCATAGTAACGCCAATAGGGACTTTCCATTGACGTCAATGGGTGGAGTATTTACGGT |
| | AAACTGCCCACTTGGCAGTACATCAAGTGTATCATATGCCAAGTCCGCCCCCTATTGACGTCAA |
| | TGACGGTAAATGGCCCGCCTGGCATTATGCCCAGTACATGACCTTACGGGACTTTCCTACTTGG |
| | CAGTACATCTACGTATTAGTCATCGCTATTACCATGGTGATGCGGTTTTGGCAGTACACCAATG |
| | GGCGTGGATAGCGGTTTGACTCACGGGGATTTCCAAGTCTCCACCCCATTGACGTCAATGGGAG |
| | TTTGTTTTGGCACCAAAATCAACGGGACTTTCCAAAATGTCGTAACAACTGCGATCGCCCGCCC |
| | CGTTGACGCAAATGGGCGGTAGGCGTGTACGGTGGGAGGTCTATATAAGCAGAGCTCGTTTAGT |
| | GAACCGTCAGATCGCCACCATGGAAGATGCCAAAAACATTAAGAAGGGCCCAGCGCCATTCTAC |
| | CCACTCGAAGACGGGACCGCCGGCGAGCAGCTCCACAAAGCCATGAAGCGCTACGCCCTGGTGC |
| | CCGGCACCATCGCCTTTACCGACGCACATATCGAGGTGGACATTACCTACGCCGAGTACTTCGA |
| | GATGAGCGTTCGGCTGGCAGAAGCTATGAAGCGCTATGGGCTGAATACAAACCATCGGATCGTG |
| | GTGTGCAGCGAGAATAGCTTGCAGTTCTTCATGCCCGTGTTGGGTGCCCTGTTCATCGGTGTGG |
| | CTGTGGCCCCAGCTAACGACATCTACAACGAGCGCGAGCTGCTGAACAGCATGGGCATCAGCCA |
| | GCCCACCGTCGTATTCGTGAGCAAGAAAGGGCTGCAAAAGATCCTCAACGTGCAAAAGAAGCTA |
| | CCGATCATACAAAAGATCATCATCATGGATAGCAAGACCGACTACCAGGGCTTCCAAAGCATGT |
| | ACACCTTCGTGACTTCCCATTTGCCACCCGGCTTCAACGAGTACGACTTCGTGCCCGAGAGCTT |
| | CGACCGGGACAAAACCATCGCCCTGATCATGAACAGTAGTGGCAGTACCGGATTGCCCAAGGGC |
| | GTAGCCCTACCGCACCGCACCGCTTGTGTCCGATTCAGTCATGCCCGCGACCCCATCTTCGGCA |
| | ACCAGATCATCCCCGACACCGCTATCCTGAGCGTGGTGCCATTTCACCACGGCTTCGGCATGTT |
| | CACCACGCTGGGCTACTTGATCTGCGGCTTTCGGGTCGTGCTCATGTACCGCTTCGAGGAGGAG |
| | CTATTCTTGCGCAGCTTGCAAGACTATAAGATTCAATCTGCCCTGCTGGTGCCCACACTATTTA |
| | GCTTCTTCGCTAAGAGCACTCTCATCGACAAGTACGACCTAAGCAACTTGCACGAGATCGCCAG |
| | CGGCGGGGCGCCGCTCAGCAAGGAGGTAGGTGAGGCCGTGGCCAAACGCTTCCACCTACCAGGC |
| | ATCCGCCAGGGCTACGGCCTGACAGAAACAACCAGCGCCATTCTGATCACCCCCGAAGGGGACG |
| | ACAAGCCTGGCGCAGTAGGCAAGGTGGTGCCCTTCTTCGAGGCTAAGGTGGTGGACTTGGACAC |
| | CGGTAAGACACTGGGTGTGAACCAGCGCGGCGAGCTGTGCGTCCGTGGCCCCATGATCATGAGC |
| | GGCTACGTTAACAACCCCGAGGCTACAAACGCTCTCATCGACAAGGACGGCTGGCTGCACAGCG |
| | GCGACATCGCCTACTGGGACGAGGACGAGCACTTCTTCATCGTGGACCGGCTGAAGTCTCTGAT |
| | CAAATACAAGGGCTACCAGGTAGCCCCAGCCGAACTGGAGAGCATCCTGCTGCAACACCCCAAC |
| | ATCTTCGACGCCGGGGTCGCCGGCCTGCCCGACGACGATGCCGGCGAGCTGCCCGCCGCAGTCG |
| | TCGTGCTGGAACACGGTAAAACCATGACCGAGAAGGAGATCGTGGACTATGTGGCCAGCCAGGT |
| | TACAACCGCCAAGAAGCTGCGCGGTGGTGTTGTGTTCGTGGACGAGGTGCCTAAAGGACTGACC |
| | GGCAAGTTGGACGCCCGCAAGATCCGCGAGATTCTCATTAAGGCCAAGAAGGGCGGCAAGATCG |
| | CCGTGGGATCCGGAGAGGGCAGAGGAAGTCTTCTAACATGCGGTGACGTGGAGGAGAATCCCGG |
| | CCCTATGGTGAGCAAGGGCGAGGAGCTGTTCACCGGGGTGGTGCCCATCCTGGTCGAGCTGGAC |
| | GGCGACGTAAACGGCCACAAGTTCAGCGTGTCCGGCGAGGGCGAGGGCGATGCCACCTACGGCA |
| | AGCTGACCCTGAAGTTCATCTGCACCACCGGCAAGCTGCCCGTGCCCTGGCCCACCCTCGTGAC |
| | CACCCTGACCTACGGCGTGCAGTGCTTCAGCCGCTACCCCGACCACATGAAGCAGCACGACTTC |
| | TTCAAGTCCGCCATGCCCGAAGGCTACGTCCAGGAGCGCACCATCTTCTTCAAGGACGACGGCA |
| | ACTACAAGACCCGCGCCGAGGTGAAGTTCGAGGGCGACACCCTGGTGAACCGCATCGAGCTGAA |
| | GGGCATCGACTTCAAGGAGGACGGCAACATCCTGGGGCACAAGCTGGAGTACAACTACAACAGC |
| | CACAACGTCTATATCATGGCCGACAAGCAGAAGAACGGCATCAAGGTGAACTTCAAGATCCGCC |
| | ACAACATCGAGGACGGCAGCGTGCAGCTCGCCGACCACTACCAGCAGAACACCCCCATCGGCGA |
| | CGGCCCCGTGCTGCTGCCCGACAACCACTACCTGAGCACCCAGTCCAAGCTGAGCAAAGACCCC |
| | AACGAGAAGCGCGATCACATGGTCCTGCTGGAGTTCGTGACCGCCGCCGGGATCACTCTCGGCA |
| | TGGACGAGCTGTACAAGTAGTGAGGATCCCCACAAAGTGACCTAGAGCTGGCAGCAAAATAAAA |
| | TATCTTTATTTTCATTACATCTGTGTGTTGGTTTTTTGTGTGAACTTCAGGAACCCCTTTTAAG |
| | AGTAACATAACTGCTGGAGTAGATGAAGATGGAGGGCCACTTTACTGGCTCTTCTAGGAACCCC |
| | TAGTGATGGAGTTGGCCACTCCCTCTCTGCGCGCTCGCTCGCTCACTGAGGCCGGGCGACCAAA |
| | GGTCGCCCGACGCCCGGGCTTTGCCCGGGCGGCCTCAGTGAGCGAGCGAGCGCGCAGAGAGGGA |
| | GTGGCCAACTCCATCACTAGGGGTTCCTAGAAGAGC |

### 7.12 Example 12. Production of AAV9 from Amplified Hairpin-Ended DNA

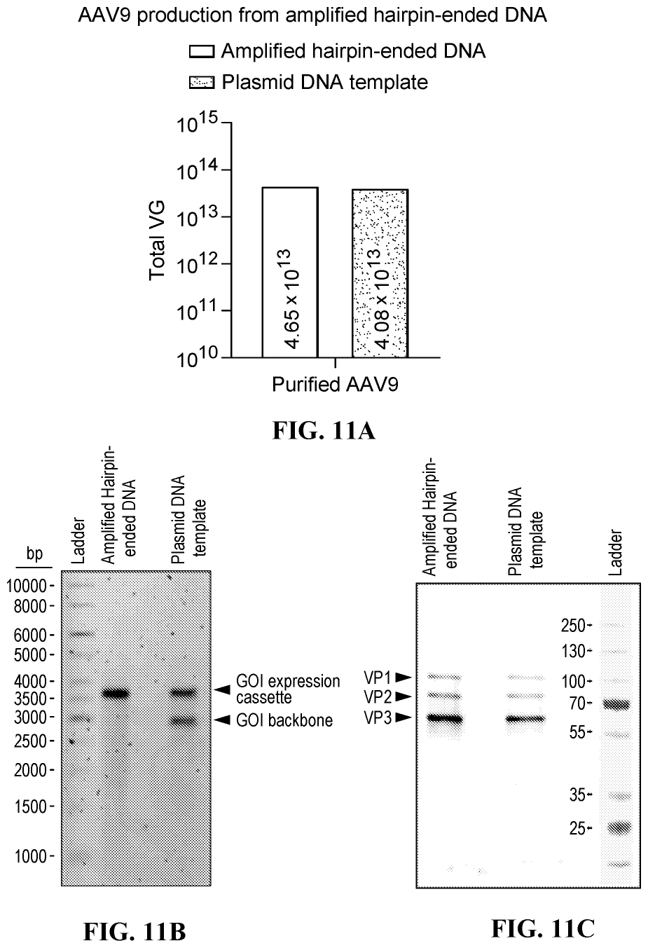

A 500 ml culture of HEK293 derived suspension cells (2.6×10⁶ cells/mL) was transfected with triple transfection system comprising amplified hairpin-ended DNA (or the corresponding plasmid DNA template), pRep2Cap9 plasmid, and pHelper plasmid as described in Example 11. 1 ug of total DNA per million cells was complexed with FectoVIR®-AAV (Polyplus; Cat No. 101000044) at a 1:1 ratio before being added to the cells. Cells and media were harvested after 72 h and AAV was purified by Affinity Chromatography and Cesium Chloride gradient. AAV-packaged viral genomes were extracted from the purified AAV stock and quantified by real time PCR with a Taqman assay against ITR sequences. AAV genome copy number within each well was calculated against an internal standard and the concentration of each stock was determined taking into consideration all the sample dilutions performed during the procedure. FIG. 11A demonstrates that amplified hairpin-ended DNA is readily used in combination with conventional RepCap of AAV9 and Helper plasmids for the production of AAV and its purification.

The AAV genome integrity and identity was assessed by running the AAV sample on an agarose gel in denaturing conditions. In brief, an alkaline agarose gel was prepared by adding 1 g agarose to 98 ml water and dissolving it in a microwave. Once the solution had cooled down, 2 ml of a 50× Alkaline Buffer (2.5 M sodium hydroxide, 50 mM EDTA) was added and the gel was poured and left to solidify. 25 μl AAV sample or diluted DNA ladder (500 ng) were mixed with 8.5 μl 4× alkaline sample loading buffer (4× Alkaline Buffer, 1.2% SDS, 20% Glycerol, 0.01% Xylene Cyanol), heated to 95° C. for 3 min, and cooled on ice prior to loading on the gel. The gel was run overnight at 20 V in a cold room in an electrophoresis apparatus filled with 1× Alkaline Running Buffer. The gel was then washed with 0.1 M Tris-HCl pH 8.5 for 1 h, before being stained in 0.1 M NaCl with 4× GelRed for 2 h. The gel is then rinsed with tap water and visualized with a UV transilluminator. The visualization is shown in FIG. 11B.

The AAV capsid proteins in each AAV sample were evaluated by western blot analysis. The samples were prepared with NuPAGE LDS sample buffer (Thermo Fisher, Cat. No NP0007) and NuPAGE Reducing Agent (Thermo Fisher, Cat No NP0004) before being loaded and run on a 4-12% (v/v) gradient, Bis-Tris, polyacrylamide gel (Thermo Fisher, Cat No NP0322BOX) with NuPAGE MOPS SDS running buffer (Thermo Fisher, Cat No NP000102). The separated proteins are transferred to a PVDF membrane from the gel using a semi dry transfer system (Trans-Blot Turbo Midi PVDF Transfer Packs, Bio-Rad, Cat. No 1704157) and detected using anti-AAV VP1/VP2/VP3 mouse antibody (clone B1, Progen, Cat No 690058) and Amersham ECL Mouse IgG, HRP-linked whole Ab (Cytiva, Cat. No NA931-1ML). The western blot analysis is shown in FIG. 11C.

### 7.13 Example 13. Characterization of DNA Content of rAAV by Next Generation Sequencing

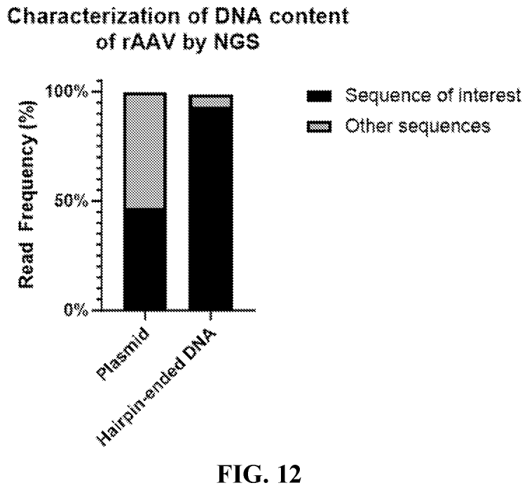

To determine the DNA content of rAAV samples (from Example 12) the Ligation Sequencing gDNA V14-Adeno-associated Virus Sequencing protocol, recommended by Oxford Nanopore Technologies (ONT) was followed (See https://community.nanoporetech.com/knowledge/know-how/AAV-seq). Briefly, viral ssDNA was extracted from rAAV particles (prepared as described in Example 12) using the PureLink Viral RNA/DNA Mini Kit (ThermoFischer #12280050) according to the manufacturer's instructions. Isolated DNA was denatured at 95° C. for 5 minutes following which plus- and minus-stranded ssDNA genomes were annealed to each other by slowly ramping the temperature down to 21° C. The resulting double-stranded DNA was purified using AMPure XP Beads (Beckman Coulter #A63881) with a ratio of 0.8× and eluted in nuclease-free water. Long-read sequencing libraries were prepared using the Native Barcoding Sequencing Kit 24 V14 (Oxford Nanopore Technologies #SQK-NBD114.24) according to the manufacturer's instructions. First, DNA ends were prepared for adaptor attachment using the NEBNext Ultra II End Repair/dA Tailing Module (NEB #E7546S). Sample-specific barcodes were then ligated to the DNA ends after which samples were pooled for multiplexing. Finally, ONT-compatible sequencing adapters were ligated to the barcoded DNA fragments. The resulting libraries were sequenced on a R10.4.1 flow cell (ONT, FLO-MIN114) using a MinION Mk1C device (ONT, MIN-101C) and MinKNOW software version 23.07.12. Raw signal data was basecalled and demultiplexed using MinKNOW selecting the “high-accuracy” model. Minimap2 was subsequently used to align sequencing reads to a concatenated reference genome that includes sequences of the human genome and of the transfection plasmids used in AAV production. Reads that passed quality thresholds based on length, basecalling and mapping qualities were then classified into the categories of sequence of interest or other sequences (including backbone, pRepCap, pHelper) (FIG. 12).

### 7.14 Example 14. Generation of Amplified Hairpin-Ended DNA for AAV Production

Three plasmid DNA templates were prepared as starting material for the generation of rAAV by triple transfection. The first plasmid DNA template comprised between the first and fourth restriction sites for nicking endonuclease and in the 5′ to 3′ direction of the top strand: a 5′ ITR; a spacer, a CMV-IE promoter; an expression cassette encoding firefly luciferase (fLuc), T2A self-cleaving peptide, and eGFP; a spacer; a polyA signal; a spacer; and a 3′ ITR (SEQ ID NO: 526. The second plasmid DNA template comprised between the first and fourth restriction sites for nicking endonuclease and in the 5′ to 3′ direction of the top strand: a 5′ ITR; a spacer, an expression cassette encoding RepCap for AAV9, a spacer; and a 3′ ITR (SEQ ID NO: 527). The third plasmid DNA template comprised between the first and fourth restriction sites for nicking endonuclease and in the 5′ to 3′ direction of the top strand: a 5′ ITR; a spacer, an expression cassette encoding Helper; a spacer; and a 3′ ITR (SEQ ID NO: 528). DNA amplification of the three DNA plasmid templates was performed according to the methods described in Example 1, but with DNA plasmid templates from between 6.4 kB to 12.1 kB. Digestion of the amplification products, formation of hairpin-ended DNA, and subsequent exonuclease digest of non-hairpin-ended DNA were performed as described in Example 1. Finally, amplified hairpin-ended DNA was purified by using a commercially available plasmid purification kit (Qiagen Plasmid Midi Kit).

TABLE 25
Sequence of Hairpin-ended DNA for AAV Production

| SEQ ID NO | Sequence |
|---|---|
| SEQ ID NO: 527 | AGAAGAGCTCTCTGTACACTCACTCACTCACTGATCCCTGGATACCAAAGGTATCCAGACAC |
| | CCAGTCTTTGACTGGGTGGGATCAGTGAGTGAGTGAGTGTACAGAGAGCTCTTCTGGAGGGA |
| | GTGGCCAAAACCAACTAGACAACTTTGTATATGAAGGCGCGCCGCAGGGTCTCCATTTTGAA |
| | GCGGGAGGTTTGAACGCGCAGCCACCATGCCGGGGTTTTACGAGATTGTGATTAAGGTCCCC |
| | AGCGACCTTGACGGGCATCTGCCCGGCATTTCTGACAGCTTTGTGAACTGGGTGGCCGAGAA |
| | GGAATGGGAGTTGCCGCCAGATTCTGACATGGATCTGAATCTGATTGAGCAGGCACCCCTGA |
| | CCGTGGCCGAGAAGCTGCAGCGCGACTTTCTGACGGAATGGCGCCGTGTGAGTAAGGCCCCG |
| | GAGGCCCTTTTCTTTGTGCAATTTGAGAAGGGAGAGAGCTACTTCCACATGCACGTGCTCGT |
| | GGAAACCACCGGGGTGAAATCCATGGTTTTGGGACGTTTCCTGAGTCAGATTCGCGAAAAAC |
| | TGATTCAGAGAATTTACCGCGGGATCGAGCCGACTTTGCCAAACTGGTTCGCGGTCACAAAG |
| | ACCAGAAATGGCGCCGGAGGCGGGAACAAGGTGGTGGATGAGTGCTACATCCCCAATTACTT |
| | GCTCCCCAAAACCCAGCCTGAGCTCCAGTGGGCGTGGACTAATATGGAACAGTATTTAAGCG |
| | CCTGTTTGAATCTCACGGAGCGTAAACGGTTGGTGGCGCAGCATCTGACGCACGTGTCGCAG |
| | ACGCAGGAGCAGAACAAAGAGAATCAGAATCCCAATTCTGATGCGCCGGTGATCAGATCAAA |
| | AACTTCAGCCAGGTACATGGAGCTGGTCGGGTGGCTCGTGGACAAGGGGATTACCTCGGAGA |
| | AGCAGTGGATCCAGGAGGACCAGGCTTCATACATCTCCTTCAATGCGGCCTCCAACTCGCGG |
| | TCCCAAATCAAGGCTGCCTTGGACAATGCGGGAAAGATTATGAGCCTGACTAAAACCGCCCC |
| | CGACTACCTGGTGGGCCAGCAGCCCGTGGAGGACATTTCCAGCAATCGGATTTATAAAATTT |
| | TGGAACTAAACGGGTACGATCCCCAATATGCGGCTTCCGTCTTTCTGGGATGGGCCACGAAA |
| | AAGTTCGGCAAGAGGAACACCATCTGGCTGTTTGGGCCTGCAACTACCGGGAAGACCAACAT |
| | CGCGGAGGCCATAGCCCACACTGTGCCCTTCTACGGGTGCGTAAACTGGACCAATGAGAACT |
| | TTCCCTTCAACGACTGTGTCGACAAGATGGTGATCTGGTGGGAGGAGGGGAAGATGACCGCC |
| | AAGGTCGTGGAGTCGGCCAAAGCCATTCTCGGAGGAAGCAAGGTGCGCGTGGACCAGAAATG |
| | CAAGTCCTCGGCCCAGATAGACCCGACTCCCGTGATCGTCACCTCCAACACCAACATGTGCG |
| | CCGTGATTGACGGGAACTCAACGACCTTCGAACACCAGCAGCCGTTGCAAGACCGGATGTTC |
| | AAATTTGAACTCACCCGCCGTCTGGATCATGACTTTGGGAAGGTCACCAAGCAGGAAGTCAA |
| | AGACTTTTTCCGGTGGGCAAAGGATCACGTGGTTGAGGTGGAGCATGAATTCTACGTCAAAA |
| | AGGGTGGAGCCAAGAAAAGACCCGCCCCCAGTGACGCAGATATAAGTGAGCCCAAACGGGTG |
| | CGCGAGTCAGTTGCGCAGCCATCGACGTCAGACGCGGAAGCTTCGATCAACTACGCAGACAG |
| | GTACCAAAACAAATGTTCTCGTCACGTGGGCATGAATCTGATGCTGTTTCCCTGCAGACAAT |
| | GCGAGAGAATGAATCAGAATTCAAATATCTGCTTCACTCACGGACAGAAAGACTGTTTAGAG |
| | TGCTTTCCCGTGTCAGAATCTCAACCCGTTTCTGTCGTCAAAAAGGCGTATCAGAAACTGTG |
| | CTACATTCATCATATCATGGGAAAGGTGCCAGACGCTTGCACTGCCTGCGATCTGGTCAATG |
| | TGGATTTGGATGACTGCATCTTTGAACAATAAATGATTTAAATCAGGTATGGCTGCCGATGG |
| | TTATCTTCCAGATTGGCTCGAGGACAACCTTAGTGAAGGAATTCGCGAGTGGTGGGCTTTGA |
| | AACCTGGAGCCCCTCAACCCAAGGCAAATCAACAACATCAAGACAACGCTCGAGGTCTTGTG |
| | CTTCCGGGTTACAAATACCTTGGACCCGGCAACGGACTCGACAAGGGGGAGCCGGTCAACGC |
| | AGCAGACGCGGCGGCCCTCGAGCACGACAAGGCTTACGACCAGCAGCTCAAGGCCGGAGACA |
| | ACCCGTACCTCAAGTACAACCACGCCGACGCCGAGTTCCAGGAGCGGCTCAAAGAAGATACG |
| | TCTTTTGGGGGCAACCTCGGGCGAGCAGTCTTCCAGGCCAAAAAGAGGCTTCTTGAACCTCT |
| | TGGTCTGGTTGAGGAAGCGGCTAAGACGGCTCCTGGAAAGAAGAGACCTGTAGAGCAGTCTC |
| | CTCAGGAACCGGACTCCTCCGCGGGTATTGGCAAATCGGGTGCACAGCCCGCTAAAAAGAGA |
| | CTCAATTTCGGTCAGACTGGCGACACAGAGTCAGTCCCAGACCCTCAACCAATCGGAGAACC |
| | TCCCGCAGCCCCCTCAGGTGTGGGATCTCTTACAATGGCTTCAGGTGGTGGCGCACCAGTGG |
| | CAGACAATAACGAAGGTGCCGATGGAGTGGGTAGTTCCTCGGGAAATTGGCATTGCGATTCC |
| | CAATGGCTGGGGGACAGAGTCATCACCACCAGCACCCGAACCTGGGCCCTGCCCACCTACAA |
| | CAATCACCTCTACAAGCAAATCTCCAACAGCACATCTGGAGGATCTTCAAATGACAACGCCT |
| | ACTTCGGCTACAGCACCCCCTGGGGGTATTTTGACTTCAACAGATTCCACTGCCACTTCTCA |
| | CCACGTGACTGGCAGCGACTCATCAACAACAACTGGGGATTCCGGCCTAAGCGACTCAACTT |
| | CAAGCTCTTTAACATTCAGGTCAAAGAGGTTACGGACAACAATGGAGTCAAGACCATCGCCA |
| | ATAACCTTACCAGCACGGTCCAGGTCTTCACGGACTCAGACTATCAGCTCCCGTACGTGCTC |
| | GGGTCGGCTCACGAGGGCTGCCTCCCGCCGTTCCCAGCGGACGTTTTCATGATTCCTCAGTA |
| | CGGGTATCTGACGCTTAATGATGGAAGCCAGGCCGTGGGTCGTTCGTCCTTTTACTGCCTGG |
| | AATATTTCCCGTCGCAAATGCTAAGAACGGGTAACAACTTCCAGTTCAGCTACGAGTTTGAG |
| | AACGTACCTTTCCATAGCAGCTACGCTCACAGCCAAAGCCTGGACCGACTAATGAATCCACT |
| | CATCGACCAATACTTGTACTATCTCTCAAAGACTATTAACGGTTCTGGACAGAATCAACAAA |
| | CGCTAAAATTCAGTGTGGCCGGACCCAGCAACATGGCTGTCCAGGGAAGAAACTACATACCT |
| | GGACCCAGCTACCGACAACAACGTGTCTCAACCACTGTGACTCAAAACAACAACAGCGAATT |
| | TGCTTGGCCTGGAGCTTCTTCTTGGGCTCTCAATGGACGTAATAGCTTGATGAATCCTGGAC |
| | CTGCTATGGCCAGCCACAAAGAAGGAGAGGACCGTTTCTTTCCTTTGTCTGGATCTTTAATT |
| | TTTGGCAAACAAGGAACTGGAAGAGACAACGTGGATGCGGACAAAGTCATGATAACCAACGA |
| | AGAAGAAATTAAAACTACTAACCCGGTAGCAACGGAGTCCTATGGACAAGTGGCCACAAACC |
| | ACCAGAGTGCCCAAGCACAGGCGCAGACCGGCTGGGTTCAAAACCAAGGAATACTTCCGGGT |
| | ATGGTTTGGCAGGACAGAGATGTGTACCTGCAAGGACCCATTTGGGCCAAAATTCCTCACAC |
| | GGACGGCAACTTTCACCCTTCTCCGCTGATGGGAGGGTTTGGAATGAAGCACCCGCCTCCTC |
| | AGATCCTCATCAAAAACACACCTGTACCTGCGGATCCTCCAACGGCCTTCAACAAGGACAAG |
| | CTGAACTCTTTCATCACCCAGTATTCTACTGGCCAAGTCAGCGTGGAGATCGAGTGGGAGCT |
| | GCAGAAGGAAAACAGCAAGCGCTGGAACCCGGAGATCCAGTACACTTCCAACTATTACAAGT |
| | CTAATAATGTTGAATTTGCTGTTAATACTGAAGGTGTATATAGTGAACCCCGCCCCATTGGC |
| | ACCAGATACCTGACTCGTAATCTGTAATTTGCTTGTTAATCAATAAACCGTTTAATTCGTTT |
| | CAGTTGAACTTTGGTCTCGCGGCCGCGTTTAAACGAGGTCCTGTATTAGAGGTCACGTGAGT |
| | GTTTTGCGACATTTTGCGACACCATGTGGTCACGCTGGGTATTTAAGCCCGAGTGAGCACGC |
| | AGGGTCTCCATTTTGAAGCGGGAGGTTTGAACGCGCATTAATTAAAACTTCAGGAACCCCTT |
| | TTAAGAGTAACATAACTGCTGGAGTAGATGAAGATGGAGGGCCACTTTACTGGCTCTTCTTC |
| | TCTGTACACTCACTCACTCACTGATCCCACCCAGTCAAAGACTGGGTGTCTGGATACCTTTG |
| | GTATCCAGGGATCAGTGAGTGAGTGAGTGTACAGAGA |
| SEQ ID NO 528 | AGAAGAGCTCTCTGTACACTCACTCACTCACTGATCCCTGGATACCAAAGGTATCCAGACAC |
| | CCAGTCTTTGACTGGGTGGGATCAGTGAGTGAGTGAGTGTACAGAGAGCTCTTCTGGAGGGA |
| | GTGGCCAAAACCAACTAGACAACTTTGTATATGAATGTACACTTAAGGGTACCCAACTCCAT |
| | GCTTAACAGTCCCCAGGTACAGCCCACCCTGCGTCGCAACCAGGAACAGCTCTACAGCTTCC |
| | TGGAGCGCCACTCGCCCTACTTCCGCAGCCACAGTGCGCAGATTAGGAGCGCCACTTCTTTT |
| | TGTCACTTGAAAAACATGTAAAAATAATGTACTAGGAGACACTTTCAATAAAGGCAAATGTT |
| | TTTATTTGTACACTCTCGGGTGATTATTTACCCCCCACCCTTGCCGTCTGCGCCGTTTAAAA |
| | ATCAAAGGGGTTCTGCCGCGCATCGCTATGCGCCACTGGCAGGGACACGTTGCGATACTGGT |
| | GTTTAGTGCTCCACTTAAACTCAGGCACAACCATCCGCGGCAGCTCGGTGAAGTTTTCACTC |
| | CACAGGCTGCGCACCATCACCAACGCGTTTAGCAGGTCGGGCGCCGATATCTTGAAGTCGCA |
| | GTTGGGGCCTCCGCCCTGCGCGCGCGAGTTGCGATACACAGGGTTGCAGCACTGGAACACTA |
| | TCAGCGCCGGGTGGTGCACGCTGGCCAGCACGCTCTTGTCGGAGATCAGATCCGCGTCCAGG |
| | TCCTCCGCGTTGCTCAGGGCGAACGGAGTCAACTTTGGTAGCTGCCTTCCCAAAAAGGGTGC |
| | ATGCCCAGGCTTTGAGTTGCACTCGCACCGTAGTGGCATCAGAAGGTGACCGTGCCCGGTCT |
| | GGGCGTTAGGATACAGCGCCTGCATGAAAGCCTTGATCTGCTTAAAAGCCACCTGAGCCTTT |
| | GCGCCTTCAGAGAAGAACATGCCGCAAGACTTGCCGGAAAACTGATTGGCCGGACAGGCCGC |
| | GTCATGCACGCAGCACCTTGCGTCGGTGTTGGAGATCTGCACCACATTTCGGCCCCACCGGT |
| | TCTTCACGATCTTGGCCTTGCTAGACTGCTCCTTCAGCGCGCGCTGCCCGTTTTCGCTCGTC |
| | ACATCCATTTCAATCACGTGCTCCTTATTTATCATAATGCTCCCGTGTAGACACTTAAGCTC |
| | GCCTTCGATCTCAGCGCAGCGGTGCAGCCACAACGCGCAGCCCGTGGGCTCGTGGTGCTTGT |
| | AGGTTACCTCTGCAAACGACTGCAGGTACGCCTGCAGGAATCGCCCCATCATCGTCACAAAG |
| | GTCTTGTTGCTGGTGAAGGTCAGCTGCAACCCGCGGTGCTCCTCGTTTAGCCAGGTCTTGCA |
| | TACGGCCGCCAGAGCTTCCACTTGGTCAGGCAGTAGCTTGAAGTTTGCCTTTAGATCGTTAT |
| | CCACGTGGTACTTGTCCATCAACGCGCGCGCAGCCTCCATGCCCTTCTCCCACGCAGACACG |
| | ATCGGCAGGCTCAGCGGGTTTATCACCGTGCTTTCACTTTCCGCTTCACTGGACTCTTCCTT |
| | TTCCTCTTGCGTCCGCATACCCCGCGCCACTGGGTCGTCTTCATTCAGCCGCCGCACCGTGC |
| | GCTTACCTCCCTTGCCGTGCTTGATTAGCACCGGTGGGTTGCTGAAACCCACCATTTGTAGC |
| | GCCACATCTTCTCTTTCTTCCTCGCTGTCCACGATCACCTCTGGGGATGGCGGGCGCTCGGG |
| | CTTGGGAGAGGGGCGCTTCTTTTTCTTTTTGGACGCAATGGCCAAATCCGCCGTCGAGGTCG |
| | ATGGCCGCGGGCTGGGTGTGCGCGGCACCAGCGCATCTTGTGACGAGTCTTCTTCGTCCTCG |
| | GACTCCAGACGCCGCCTCAGCCGCTTTTTTGGGGGCGCGCGGGGAGGCGGCGGCGACGGCGA |
| | CGGGGACGACACGTCCTCCATGGTTGGTGGACGTCGCGCCGCACCGCGTCCGCGCTCGGGGG |
| | TGGTTTCGCGCTGCTCCTCTTCCCGACTGGCCATTTCCTTCTCCTATAGGCAGAAAAAGATC |
| | ATGGAGTCAGTCGAGAAGGAGGACAGCCTAACCGCCCCCTTTGAGTTCGCCACCACCGCCTC |
| | CACCGATGCCGCCAACGCGCCTACCACCTTCCCCGTCGAGGCACCCCCGCTTGAGGAGGAGG |
| | AAGTGATTATCGAGCAGGACCCAGGTTTTGTAAGCGAAGACGACGAGGATCGCTCAGTACCA |
| | ACAGAGGATAAAAAGCAAGACCAGGACGACGCAGAGGCAAACGAGGAACAAGTCGGGGGGGG |
| | GGACCAAAGGCATGGCGACTACCTAGATGTGGGCGACGACGTGCTGTTGAAGCATCTGCAGC |
| | GCCAGTGCGCCATTATCTGCGACGCGTTGCAAGAGCGCAGCGATGTGCCCCTCGCCATAGCG |
| | GATGTCAGCCTTGCCTACGAACGCCACCTGTTCTCACCGCGCGTACCCCCCAAACGCCAAGA |
| | AAACGGCACATGCGAGCCCAACCCGCGCCTCAACTTCTACCCCGTATTTGCCGTGCCAGAGG |
| | TGCTTGCCACCTATCACATCTTTTTCCAAAACTGCAAGATACCCCTATCCTGCCGTGCCAAC |
| | CGCAGCCGAGCGGACAAGCAGCTGGCCTTGCGGCAGGGCGCTGTCATACCTGATATCGCCTC |
| | GCTCGACGAAGTGCCAAAAATCTTTGAGGGTCTTGGACGCGACGAGAAACGCGCGGCAAACG |
| | CTCTGCAACAAGAAAACAGCGAAAATGAAAGTCACTGTGGAGTGCTGGTGGAACTTGAGGGT |
| | GACAACGCGCGCCTAGCCGTGCTGAAACGCAGCATCGAGGTCACCCACTTTGCCTACCCGGC |
| | ACTTAACCTACCCCCCAAGGTTATGAGCACAGTCATGAGCGAGCTGATCGTGCGCCGTGCAC |
| | GACCCCTGGAGAGGGATGCAAACTTGCAAGAACAAACCGAGGAGGGCCTACCCGCAGTTGGC |
| | GATGAGCAGCTGGCGCGCTGGCTTGAGACCCGCGAGCCTGCCGACTTGGAGGAGCGACGCAA |
| | GCTAATGATGGCCGCAGTGCTTGTTACCGTGGAGCTTGAGTGCATGCAGCGGTTCTTTGCTG |
| | ACCCGGAGATGCAGCGCAAGCTAGAGGAAACGTTGCACTACACCTTTCGCCAGGGCTACGTG |
| | CGCCAGGCTTGCAAAATTTCCAACGTGGAGCTCTGCAACCTGGTCTCCTACCTTGGAATTTT |
| | GCACGAAAACCGCCTCGGGCAAAACGTGCTTCATTCCACGCTCAAGGGCGAGGCGCGCCGCG |
| | ACTACGTCCGCGACTGCGTTTACTTATTTCTGTGCTACACCTGGCAAACGGCCATGGGCGTG |
| | TGGCAGCAATGCCTGGAGGAGCGCAACCTAAAGGAGCTGCAGAAGCTGCTAAAGCAAAACTT |
| | GAAGGACCTATGGACGGCCTTCAACGAGCGCTCCGTGGCCGCGCACCTGGCGGACATTATCT |
| | TCCCCGAACGCCTGCTTAAAACCCTGCAACAGGGTCTGCCAGACTTCACCAGTCAAAGCATG |
| | TTGCAAAACTTTAGGAACTTTATCCTAGAGCGTTCAGGAATTCTGCCCGCCACCTGCTGTGC |
| | GCTTCCTAGCGACTTTGTGCCCATTAAGTACCGTGAATGCCCTCCGCCGCTTTGGGGTCACT |
| | GCTACCTTCTGCAGCTAGCCAACTACCTTGCCTACCACTCCGACATCATGGAAGACGTGAGC |
| | GGTGACGGCCTACTGGAGTGTCACTGTCGCTGCAACCTATGCACCCCGCACCGCTCCCTGGT |
| | CTGCAATTCGCAACTGCTTAGCGAAAGTCAAATTATCGGTACCTTTGAGCTGCAGGGTCCCT |
| | CGCCTGACGAAAAGTCCGCGGCTCCGGGGTTGAAACTCACTCCGGGGCTGTGGACGTCGGCT |
| | TACCTTCGCAAATTTGTACCTGAGGACTACCACGCCCACGAGATTAGGTTCTACGAAGACCA |
| | ATCCCGCCCGCCAAATGCGGAGCTTACCGCCTGCGTCATTACCCAGGGCCACATCCTTGGCC |
| | AATTGCAAGCCATCAACAAAGCCCGCCAAGAGTTTCTGCTACGAAAGGGACGGGGGGTTTAC |
| | CTGGACCCCCAGTCCGGCGAGGAGCTCAACCCAATCCCCCCGCCGCCGCAGCCCTATCAGCA |
| | GCCGCGGGCCCTTGCTTCCCAGGATGGCACCCAAAAAGAAGCTGCAGCTGCCGCCGCCGCCA |
| | CCCACGGACGAGGAGGAATACTGGGACAGTCAGGCAGAGGAGGTTTTGGACGAGGAGGAGGA |
| | GATGATGGAAGACTGGGACAGCCTAGACGAAGCTTCCGAGGCCGAAGAGGTGTCAGACGAAA |
| | CACCGTCACCCTCGGTCGCATTCCCCTCGCCGGCGCCCCAGAAATTGGCAACCGTTCCCAGC |
| | ATCGCTACAACCTCCGCTCCTCAGGCGCCGCCGGCACTGCCTGTTCGCCGACCCAACCGTAG |
| | ATGGGACACCACTGGAACCAGGGCCGGTAAGTCTAAGCAGCCGCCGCCGTTAGCCCAAGAGC |
| | AACAACAGCGCCAAGGCTACCGCTCGTGGCGCGGGCACAAGAACGCCATAGTTGCTTGCTTG |
| | CAAGACTGTGGGGGCAACATCTCCTTCGCCCGCCGCTTTCTTCTCTACCATCACGGCGTGGC |
| | CTTCCCCCGTAACATCCTGCATTACTACCGTCATCTCTACAGCCCCTACTGCACCGGCGGCA |
| | GCGGCAGCGGCAGCAACAGCAGCGGTCACACAGAAGCAAAGGCGACCGGATAGCAAGACTCT |
| | GACAAAGCCCAAGAAATCCACAGCGGCGGCAGCAGCAGGAGGAGGAGCGCTGCGTCTGGCGC |
| | CCAACGAACCCGTATCGACCCGCGAGCTTAGAAATAGGATTTTTCCCACTCTGTATGCTATA |
| | TTTCAACAAAGCAGGGGCCAAGAACAAGAGCTGAAAATAAAAAACAGGTCTCTGCGCTCCCT |
| | CACCCGCAGCTGCCTGTATCACAAAAGCGAAGATCAGCTTCGGCGCACGCTGGAAGACGCGG |
| | AGGCTCTCTTCAGCAAATACTGCGCGCTGACTCTTAAGGACTAGTTTCGCGCCCTTTCTCAA |
| | ATTTAAGCGCGAAAACTACGTCATCTCCAGCGGCCACACCCGGCGCCAGCACCTGTCGTCAG |
| | CGCCATTATGAGCAAGGAAATTCCCACGCCCTACATGTGGAGTTACCAGCCACAAATGGGAC |
| | TTGCGGCTGGAGCTGCCCAAGACTACTCAACCCGAATAAACTACATGAGCGCGGGACCCCAC |
| | ATGATATCCCGGGTCAACGGAATCCGCGCCCACCGAAACCGAATTCTCCTCGAACAGGCGGC |
| | TATTACCACCACACCTCGTAATAACCTTAATCCCCGTAGTTGGCCCGCTGCCCTGGTGTACC |
| | AGGAAAGTCCCGCTCCCACCACTGTGGTACTTCCCAGACACGCCCAGGCCGAAGTTCAGATG |
| | ACTAACTCAGGGGCGCAGCTTGCGGGCGGCTTTCGTCACAGGGTGCGGTCGCCCGGGCGTTT |
| | TAGGGCGGAGTAACTTGCATGTATTGGGAATTGTAGTTTTTTTAAAATGGGAAGTGACGTAT |
| | CGTGGGAAAACGGAAGTGAAGATTTGAGGAAGTTGTGGGTTTTTTGGCTTTCGTTTCTGGGC |
| | GTAGGTTCGCGTGCGGTTTTCTGGGTGTTTTTTGTGGACTTTAACCGTTACGTCATTTTTTA |
| | GTCCTATATATACTCGCTCTGTACTTGGCCCTTTTTACACTGTGACTGATTGAGCTGGTGCC |
| | GTGTCGAGTGGTGTTTTTTAATAGGTTTTTTTACTGGTAAGGCTGACTGTTATGGCTGCCGC |
| | TGTGGAAGCGCTGTATGTTGTTCTGGAGCGGGAGGGTGCTATTTTGCCTAGGCAGGAGGGTT |
| | TTTCAGGTGTTTATGTGTTTTTCTCTCCTATTAATTTTGTTATACCTCCTATGGGGGCTGTA |
| | ATGTTGTCTCTACGCCTGCGGGTATGTATTCCCCCGGGCTATTTCGGTCGCTTTTTAGCACT |
| | GACCGATGTTAACCAACCTGATGTGTTTACCGAGTCTTACATTATGACTCCGGACATGACCG |
| | AGGAACTGTCGGTGGTGCTTTTTAATCACGGTGACCAGTTTTTTTACGGTCACGCCGGCATG |
| | GCCGTAGTCCGTCTTATGCTTATAAGGGTTGTTTTTCCTGTTGTAAGACAGGCTTCTAATGT |
| | TTAAATGTTTTTTTTTTTGTTATTTTATTTTGTGTTTAATGCAGGAACCCGCAGACATGTTT |
| | GAGAGAAAAATGGTGTCTTTTTCTGTGGTGGTTCCGGAACTTACCTGCCTTTATCTGCATGA |
| | GCATGACTACGATGTGCTTGCTTTTTTGCGCGAGGCTTTGCCTGATTTTTTGAGCAGCACCT |
| | TGCATTTTATATCGCCGCCCATGCAACAAGCTTACATAGGGGCTACGCTGGTTAGCATAGCT |
| | CCGAGTATGCGTGTCATAATCAGTGTGGGTTCTTTTGTCATGGTTCCTGGCGGGGAAGTGGC |
| | CGCGCTGGTCCGTGCAGACCTGCACGATTATGTTCAGCTGGCCCTGCGAAGGGACCTACGGG |
| | ATCGCGGTATTTTTGTTAATGTTCCGCTTTTGAATCTTATACAGGTCTGTGAGGAACCTGAA |
| | TTTTTGCAATCATGATTCGCTGCTTGAGGCTGAAGGTGGAGGGCGCTCTGGAGCAGATTTTT |
| | ACAATGGCCGGACTTAATATTCGGGATTTGCTTAGAGACATATTGATAAGGTGGCGAGATGA |
| | AAATTATTTGGGCATGGTTGAAGGTGCTGGAATGTTTATAGAGGAGATTCACCCTGAAGGGT |
| | TTAGCCTTTACGTCCACTTGGACGTGAGGGCAGTTTGCCTTTTGGAAGCCATTGTGCAACAT |
| | CTTACAAATGCCATTATCTGTTCTTTGGCTGTAGAGTTTGACCACGCCACCGGAGGGGAGCG |
| | CGTTCACTTAATAGATCTTCATTTTGAGGTTTTGGATAATCTTTTGGAATAAAAAAAAAAAA |
| | ACATGGTTCTTCCAGCTTCTCCCGCTCCTCCCGTGTGTGACTCGCAGAACGAATGTGTAGGT |
| | TGGCTGGGTGTGGCTTATTCTGCGGTGGTGGATGTTATCAGGGCAGCGGCGCATGAAGGAGT |
| | TTACATAGAACCCGAAGCCAGGGGGCGCCTGGATGCTTTGAGAGAGTGGATATACTACAACT |
| | ACTACACAGAGCGAGCTAAGCGACGAGACCGGAGATGCAGATCTGTTTGTCACGCCCGCACC |
| | TGGTTTTGCTTCAGGAAATATGACTACGTCCGGCGTTCCATTTGGCATGACACTACGACCAA |
| | CACGATCTCGGTTGTCTCGGCGCACTCCGTACAGTAGGGATCGCCTACCTCCTTTTGAGACA |
| | GAGACCCGCGCTACCATACTGGAGGATCATCCGCTGCTGCCCGAATGTAACACTTTGACAAT |
| | GCACAACGTGAGTTACGTGCGAGGTCTTCCCTGCAGTGTGGGATTTACGCTGATTCAGGAAT |
| | GGGTTGTTCCCTGGGATATGGTTCTGACGCGGGAGGAGCTTGTAATCCTGAGGAAGTGTATG |
| | CACGTGTGCCTGTGTTGTGCCAACATTGATATCATGACGAGCATGATGATCCATGGTTACGA |
| | GTCCTGGGCTCTCCACTGTCATTGTTCCAGTCCCGGTTCCCTGCAGTGCATAGCCGGCGGGC |
| | AGGTTTTGGCCAGCTGGTTTAGGATGGTGGTGGATGGCGCCATGTTTAATCAGAGGTTTATA |
| | TGGTACCGGGAGGTGGTGAATTACAACATGCCAAAAGAGGTAATGTTTATGTCCAGCGTGTT |
| | TATGAGGGGTCGCCACTTAATCTACCTGCGCTTGTGGTATGATGGCCACGTGGGTTCTGTGG |
| | TCCCCGCCATGAGCTTTGGATACAGCGCCTTGCACTGTGGGATTTTGAACAATATTGTGGTG |
| | CTGTGCTGCAGTTACTGTGCTGATTTAAGTGAGATCAGGGTGCGCTGCTGTGCCCGGAGGAC |
| | AAGGAGACTCATGCTGCGGGCGGTGCGAATCATCGCTGAGGAGACCACTGCCATGTTGTATT |
| | CCTGCAGGACGGAGCGGCGGCGGCAGCAGTTTATTCGCGCGCTGCTGCAGCACCACCGCCCT |
| | ATCCTGATGCACGATTATGACTCTACCCCCATGTAGGCGTGGACTTCCCCTTCGCCGCCCGT |
| | TGAGCAACCGCAAGTTGGACAGCAGCCTGTGGCTCAGCAGCTGGACAGCGACATGAACTTAA |
| | GCGAGCTGCCCGGGGAGTTTATTAATATCACTGATGAGCGTTTGGCTCGACAGGAAACCGTG |
| | TGGAATATAACACCTAAGAATATGTCTGTTACCCATGATATGATGCTTTTTAAGGCCAGCCG |
| | GGGAGAAAGGACTGTGTACTCTGTGTGTTGGGAGGGAGGTGGCAGGTTGAATACTAGGGTTC |
| | TGTGAGTTTGATTAAGGTACGGTGATCAATATAAGCTATGTGGTGGTGGGGCTATACTACTG |
| | AATGAAAAATGACTTGAAATTTTCTGCAATTGAAAAATAAACACGTTGAAACATAACATGCA |
| | ACAGGTTCACGATTCTTTATTCCTGGGCAATGTAGGAGAAGGTGTAAGAGTTGGTAGCAAAA |
| | GTTTCAGTGGTGTATTTTCCACTTTCCCAGGACCATGTAAAAGACATAGAGTAAGTGCTTAC |
| | CTCGCTAGTTTCTGTGGATTCACTAGAATGGATGTAGGATGTTGCCCCTCCTGACGCGGTAG |
| | GAGAAGGGGAGGGTGCCCTGCATGTCTGCCGCTGCTCTTGCTCTTGCCGCTGCTGAGGAGGG |
| | GGGCGCATCTGCCGCAGCACCGGATGCATCTGGGAAAAGCAAAAAAGGGGCTCGTCCCTGTT |
| | TCCGGAGGAATTTGCAAGCGGGGTCTTGCATGACGGGGAGGCAAACCCCCGTTCGCCGCAGT |
| | CCGGCCGGCCCGAGACTCGAACCGGGGGTCCTGCGACTCAACCCTTGGAAAATAACCCTCCG |
| | GCTACAGGGAGCGAGCCACTTAATGCTTTCGCTTTCCAGCCTAACCGCTTACGCCGCGCGCG |
| | GCCAGTGGCCAAAAAAGCTAGCGCAGCAGCCGCCGCGCCTGGAAGGAAGCCAAAAGGAGCGC |
| | TCCCCCGTTGTCTGACGTCGCACACCTGGGTTCGACACGCGGGCGGTAACCGCATGGATCAC |
| | GGCGGACGGCCGGATACGGGGCTCGAACCCCGGTCGTCCGCCATGATACCCTTGCGAATTTA |
| | TCCACCAGACCACGGAAGAGTGCCCGCTTACAGGCTCTCCTTTTGCACGGTCTAGAGCGTCA |
| | ACGACTGCGCACGCCTCACCGGCCAGAGCGTCCCGACCATGGAGCACTTTTTGCCGCTGCGC |
| | AACATCTGGAACCGCGTCCGCGACTTTCCGCGCGCCTCCACCACCGCCGCCGGCATCACCTG |
| | GATGTCCAGGTACATCTACGGATTACGTGCTAGCAGATCTACTAGTAACTTCAGGAACCCCT |
| | TTTAAGAGTAACATAACTGCTGGAGTAGATGAAGATGGAGGGCCACTTTACTGGCTCTTCTT |
| | CTCTGTACACTCACTCACTCACTGATCCCACCCAGTCAAAGACTGGGTGTCTGGATACCTTT |
| | GGTATCCAGGGATCAGTGAGTGAGTGAGTGTACAGAGA |
| SEQ ID NO 529 | AGGAACCCCTAGTGATGGAGTTGGCCACTCCCTCTCTGCGCGCTCGCTCGCTCACTGAGGCC |
| | GGGCGACCAAAGGTCGCCCGACGCCCGGGCTTTGCCCGGGCGGCCTCAGTGAGCGAGCGAGC |
| | GCGCAGAGAGGGAGTGGCCAACTCCATCACTAGGGGTTCCT |
| SEQ ID NO 530 | AGGAACCCCTAGTGATGGAGTTGGCCACTCCCTCTCTGCGCGCTCGCTCGCTCACTGAGGCC |
| | GGGCGACCAAAGGTCGCCCGACGCCCGGGCTTTGCCCGGGCGGCCTCAGTGAGCGAGCGAGC |
| | GCGCAGAGAGGGAGTGGCCAACTCCATCACTAGGGGTTCCT |

### 7.15 Example 15. AAV9 Production by Triple Transfection with Amplified Hairpin-Ended DNA

HEK293T cells were seeded in 6-well plates at a density of 670,000 cells/well in standard growth media supplemented with 10% fetal bovine serum (FBS) and left to adhere overnight at 37° C., 5% CO₂. After 24 hours, the media was changed to low serum media (2% FBS) and cells were transfected with three amplified hairpin-ended DNA constructs prepared in Example 14 (SEQ ID NOs: 526, 527, and 528) at an optimized molar ratio (1:4:1), or the three corresponding plasmid DNA templates at a molar ratio of 1:1:1. Transfection complexes were added to the cells after incubation of 1 ug total DNA with 1 ul of Fecto VIR®-AAV (Polyplus; Cat No. 101000044) for 30 min at room temperature. At 24 hours post-transfection, the media was changed to standard growth media and cells incubated at 37° C. for another 48 h before being harvested. In brief, cells and their culture media were collected from each well into a 2 ml tube, and centrifuged at 300 g for 5 min. Without disturbing the cell pellet, the media was transferred into a new tube and filtered with a 0.45 um PES filter (Cytiva, Cat No. 6780-2504) before being stored.

AAV-packaged viral genomes were extracted from collected media and quantified by real time PCR with a Taqman assay against the reporter sequence. In brief, the media was digested with DNAse I (Roche, Cat No. 04716728001), in triplicate, following the manufacturer's instruction. Upon DNAse I inactivation at 70° C. for 10 min, the reaction mix was subjected to Proteinase K (Roche, Cat No. 3115887001) treatment at 50° C. for 2 h before enzyme inactivation at 95° C. in 10 min. Each Proteinase K-treated sample was used as template in a titration qPCR where the AAV genome copy number within each well was calculated against an internal standard of known quantity. The viral genome concentration for each stock was determined taking into consideration all the sample dilutions performed during the procedure.

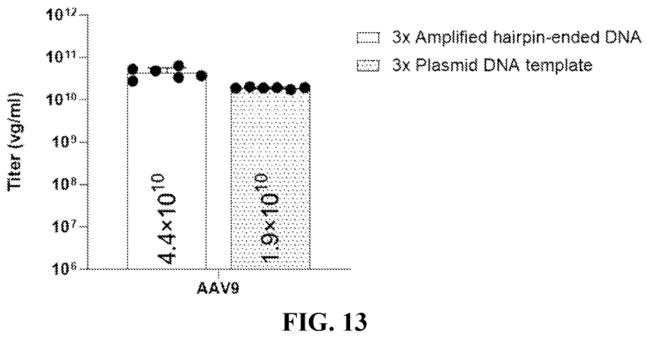

FIG. 13 demonstrates that amplified hairpin-ended DNA is readily used with Fecto VIR®-AAV for the production of AAV, that is generated at higher titer than conventional methods.

### 7.16 Example 16. Generation of Amplified Hairpin-Ended DNA for Cap1-mRNA Production by IVT

A plasmid DNA template, comprising between the first and fourth restriction sites for nicking endonuclease and in the 5′ to 3′ direction of the top strand: a 5′ ITR; a T7 Cap1 promoter; an expression cassette encoding firefly luciferase (fLuc), T2A self-cleaving peptide, and eGFP; a spacer; a sequence encoding polyA; a T7 terminator; a spacer; and a 3′ ITR (SEQ ID NO: 531), was prepared as starting material for the generation of transcription ready hairpin-ended DNA. DNA amplification was performed according to the methods described in Example 1, but with a circular DNA template of around 5.4 kB. Digestion of the amplification product, formation of hairpin-ended DNA, and subsequent exonuclease digestion of non-hairpin-ended DNA were performed as described in Example 1. Finally, amplified hairpin-ended DNA was purified by using a commercially available plasmid purification kit (Qiagen Plasmid Midi Kit).

TABLE 26
Sequence of Hairpin-ended DNA for mRNA Production

| SEQ ID NO | Sequence |
|---|---|
| SEQ ID NO: 531 | TCTCTGTACACTCACTCACTCACTGATCCCTGGATACCAAAGGTATCCAGACACCCAGTCTTTG |
| | ACTGGGTGGGATCAGTGAGTGAGTGAGTGTACAGAGAAGAAGAGCCAGTGGAGTGGCCAAAACC |
| | AACTAGACAGGTGAGATATCTAATAATACGACTCACTATAAGGAAATAAGAGAGAAAAGAAGAG |
| | TAAGAAGAAATATAAGAGCCACCATGGAAGATGCCAAAAACATTAAGAAGGGCCCAGCGCCATT |
| | CTACCCACTCGAAGACGGGACCGCCGGCGAGCAGCTCCACAAAGCCATGAAGCGCTACGCCCTG |
| | GTGCCCGGCACCATCGCCTTTACCGACGCACATATCGAGGTGGACATTACCTACGCCGAGTACT |
| | TCGAGATGAGCGTTCGGCTGGCAGAAGCTATGAAGCGCTATGGGCTGAATACAAACCATCGGAT |
| | CGTGGTGTGCAGCGAGAATAGCTTGCAGTTCTTCATGCCCGTGTTGGGTGCCCTGTTCATCGGT |
| | GTGGCTGTGGCCCCAGCTAACGACATCTACAACGAGCGCGAGCTGCTGAACAGCATGGGCATCA |
| | GCCAGCCCACCGTCGTATTCGTGAGCAAGAAAGGGCTGCAAAAGATCCTCAACGTGCAAAAGAA |
| | GCTACCGATCATACAAAAGATCATCATCATGGATAGCAAGACCGACTACCAGGGCTTCCAAAGC |
| | ATGTACACCTTCGTGACTTCCCATTTGCCACCCGGCTTCAACGAGTACGACTTCGTGCCCGAGA |
| | GCTTCGACCGGGACAAAACCATCGCCCTGATCATGAACAGTAGTGGCAGTACCGGATTGCCCAA |
| | GGGCGTAGCCCTACCGCACCGCACCGCTTGTGTCCGATTCAGTCATGCCCGCGACCCCATCTTC |
| | GGCAACCAGATCATCCCCGACACCGCTATCCTGAGCGTGGTGCCATTTCACCACGGCTTCGGCA |
| | TGTTCACCACGCTGGGCTACTTGATCTGCGGCTTTCGGGTCGTGCTCATGTACCGCTTCGAGGA |
| | GGAGCTATTCTTGCGCAGCTTGCAAGACTATAAGATTCAATCTGCCCTGCTGGTGCCCACACTA |
| | TTTAGCTTCTTCGCTAAGAGCACTCTCATCGACAAGTACGACCTAAGCAACTTGCACGAGATCG |
| | CCAGCGGCGGGGCGCCGCTCAGCAAGGAGGTAGGTGAGGCCGTGGCCAAACGCTTCCACCTACC |
| | AGGCATCCGCCAGGGCTACGGCCTGACAGAAACAACCAGCGCCATTCTGATCACCCCCGAAGGG |
| | GACGACAAGCCTGGCGCAGTAGGCAAGGTGGTGCCCTTCTTCGAGGCTAAGGTGGTGGACTTGG |
| | ACACCGGTAAGACACTGGGTGTGAACCAGCGCGGCGAGCTGTGCGTCCGTGGCCCCATGATCAT |
| | GAGCGGCTACGTTAACAACCCCGAGGCTACAAACGCTCTCATCGACAAGGACGGCTGGCTGCAC |
| | AGCGGCGACATCGCCTACTGGGACGAGGACGAGCACTTCTTCATCGTGGACCGGCTGAAGTCTC |
| | TGATCAAATACAAGGGCTACCAGGTAGCCCCAGCCGAACTGGAGAGCATCCTGCTGCAACACCC |
| | CAACATCTTCGACGCCGGGGTCGCCGGCCTGCCCGACGACGATGCCGGCGAGCTGCCCGCCGCA |
| | GTCGTCGTGCTGGAACACGGTAAAACCATGACCGAGAAGGAGATCGTGGACTATGTGGCCAGCC |
| | AGGTTACAACCGCCAAGAAGCTGCGCGGTGGTGTTGTGTTCGTGGACGAGGTGCCTAAAGGACT |
| | GACCGGCAAGTTGGACGCCCGCAAGATCCGCGAGATTCTCATTAAGGCCAAGAAGGGCGGCAAG |
| | ATCGCCGTGGGATCCGGAGAGGGCAGAGGAAGTCTTCTAACATGCGGTGACGTGGAGGAGAATC |
| | CCGGCCCTATGGTGAGCAAGGGCGAGGAGCTGTTCACCGGGGTGGTGCCCATCCTGGTCGAGCT |
| | GGACGGCGACGTAAACGGCCACAAGTTCAGCGTGTCCGGCGAGGGCGAGGGCGATGCCACCTAC |
| | GGCAAGCTGACCCTGAAGTTCATCTGCACCACCGGCAAGCTGCCCGTGCCCTGGCCCACCCTCG |
| | TGACCACCCTGACCTACGGCGTGCAGTGCTTCAGCCGCTACCCCGACCACATGAAGCAGCACGA |
| | CTTCTTCAAGTCCGCCATGCCCGAAGGCTACGTCCAGGAGCGCACCATCTTCTTCAAGGACGAC |
| | GGCAACTACAAGACCCGCGCCGAGGTGAAGTTCGAGGGCGACACCCTGGTGAACCGCATCGAGC |
| | TGAAGGGCATCGACTTCAAGGAGGACGGCAACATCCTGGGGCACAAGCTGGAGTACAACTACAA |
| | CAGCCACAACGTCTATATCATGGCCGACAAGCAGAAGAACGGCATCAAGGTGAACTTCAAGATC |
| | CGCCACAACATCGAGGACGGCAGCGTGCAGCTCGCCGACCACTACCAGCAGAACACCCCCATCG |
| | GCGACGGCCCCGTGCTGCTGCCCGACAACCACTACCTGAGCACCCAGTCCAAGCTGAGCAAAGA |
| | CCCCAACGAGAAGCGCGATCACATGGTCCTGCTGGAGTTCGTGACCGCCGCCGGGATCACTCTC |
| | GGCATGGACGAGCTGTACAAGTAGTGAGGATCCCCACAAAGTGACCTAGAGCTGGCAGCAAAAA |
| | AAAAAAAAAAAAAAAAAAAAAAAAAAAGAGACCCTAGCATAACCCCTTGGGGCCTCTAAACGGG |
| | TCTTGAGGGGTTTTTTGAACTGATATCACCCCTTTTAAGAGTAACATAACTGCTGGAGTAGATG |
| | AAGATGGAGGGCCACTTTCTCCAGAAGAGCTCTCTGTACACTCACTCACTCACTGATCCCACCC |
| | AGTCAAAGACTGGGTGTCTGGATACCTTTGGTATCCAGGGATCAGTGAGTGAGTGAGTGTACAG |
| | AGAGCTCTTCT |

### 7.17 Example 17. IVT of Amplified Hairpin-Ended DNA to Produce Cap1-mRNA with Modified and Unmodified Nucleotides

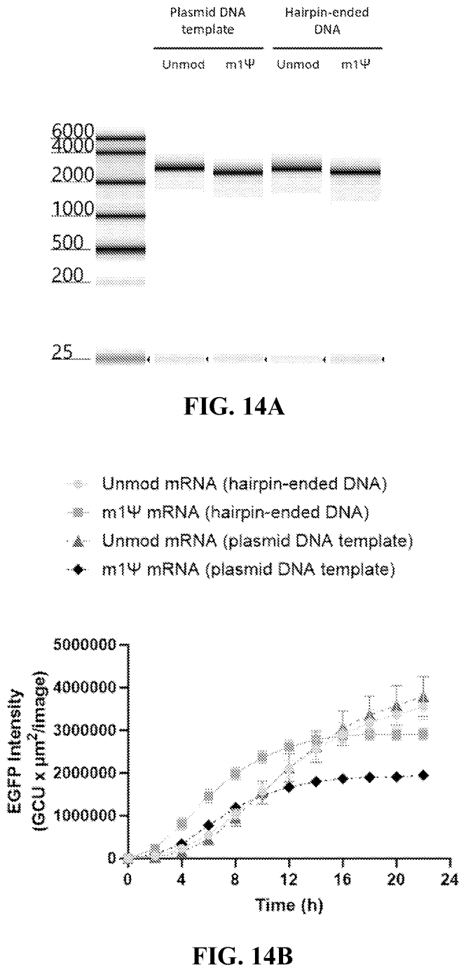

Twelve (12) μg of amplified hairpin-ended DNA molecules produced as in Example 16, or the corresponding plasmid DNA template, were linearized with the restriction enzyme EcoRV-HF (NEB; Cat No. R3195L) in 5 μl of NEB 3.1 buffer 10× in water at a final volume of 50 μl. Digested DNA templates were purified with NucleoSpin Gel and PCR Clean-up kit from Takara (Cat No. 740609.50). An IVT reaction (20 μL) was prepared for each DNA sample using the HiScribe® T7 High Yield RNA Synthesis Kit (NEB; Cat No. E2040S). In brief, a mixture was prepared for each DNA sample by combining 0.29 μmol of digested DNA template, 3.75 mM CleanCap AG (Trilink, Cat No. N-7113), 7.5 mM ATP, 7.5 mM GTP, 7.5 mM CTP, 7.5 mM of either UTP or m1ΨTP, 0.5 μl of RNasin Ribonuclease Inhibitor (Promega; Cat No. N251A) and 2 μl of T7 RNA polymerase mix. The reaction was incubated at 37° C. for 2 hours. Then, 2 μL of Dnase I (NEB; Cat no. M0303S), 10 μL DNase I reaction buffer (NEB; Cat No. B0303S) and 70 μL of nuclease-free water were added to the mixture and incubated at 37° C. for 15 minutes. Product mRNA was subsequently purified by lithium chloride (LiCl) precipitation. 50 μL of LiCl precipitation solution (ThermoFisher; Cat No. AM9480) were added to the mix. The mixture was incubated for 30 minutes at −20° C. Samples were washed twice by centrifugation for 15 minutes at 4° C. followed by a wash with ice-cold 70% ethanol (500 μL). Finally, the mRNA pellets were air-dried and resuspended in RNA buffer. The quality and quantification of the final mRNA produced from linearized plasmid DNA and amplified hairpin ended DNA templates were analyzed using an Agilent 4200 TapeStation System and an RNA Assay (Cat Nos. 5067-5576, 5067-5577) following the manufacturer's instructions (FIG. 14A). For functional validations, HEK293T cells were seeded in 96-well plates at a density of 20,000 cells/well in standard growth media supplemented with 10% fetal bovine serum and left to adhere overnight. After 24 hours, media was exchanged, and cells were transfected with the mRNAs either from hairpin-ended DNA or from plasmid DNA. The mRNA was mixed with in Lipofectamine MessengerMAX (ThermoFisher Scientific, Cat No. LMRNA001) following manufacturer's protocol and added to the cells, in triplicates, at 100 ng/well. EGFP expression was monitored over 22 hours post-transfection in an Incucyte SX5 (Sartorius) (FIG. 14B)

### 7.18 Example 18. Methods of Digesting Amplified Hairpin-Ended DNA for IVT to Produce Cap1-mRNA

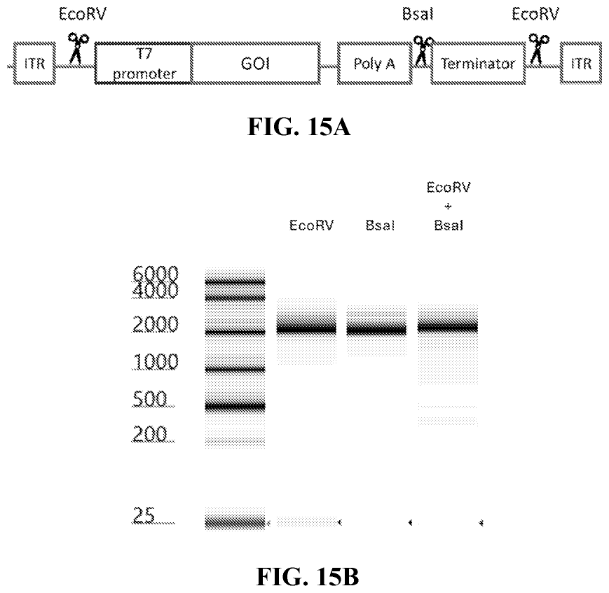

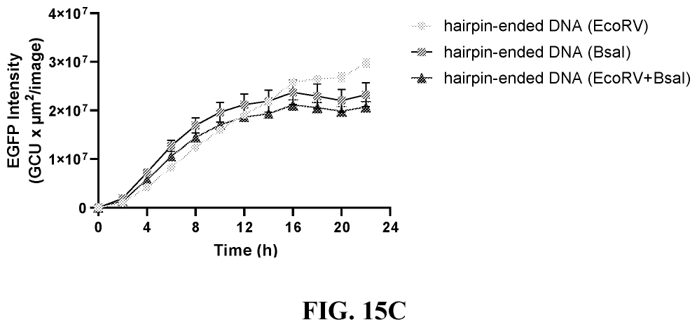

Twelve (12) μg of amplified hairpin-ended DNA molecules produced as in Example 16 was linearized with 2 μL of either EcoRV-HF (NEB; Cat No. R3195L), BsaI-HF v2 (NEB; Cat No. R3733L) or a combination of both (2 μL of each enzyme) in 5 μl of NEB 3.1 buffer 10× in water at a final volume of 50 μl. The cutting sites within the hairpin-ended DNA are shown in FIG. 15A. Digested DNA templates were purified with NucleoSpin Gel and PCR Clean-up kit from Takara (Cat No. 740609.50). An IVT reaction (20 μL) was prepared for each DNA sample using the HiScribe® T7 High Yield RNA Synthesis Kit (NEB; Cat No. E2040S). In brief, a mixture was prepared for each DNA sample by combining 0.29 μmol of digested DNA template, 3.75 mM CleanCap AG (Trilink, Cat No. N-7113), 7.5 mM ATP, 7.5 mM GTP, 7.5 mM CTP, 5 mM of UTP and 2.5 mM m1ΨTP, 0.5 μl of RNasin Ribonuclease Inhibitor (Promega; Cat No. N251A) and 2 μl of T7 RNA polymerase mix, The reaction was incubated at 37° C. for 2 hours. Then, 2 μL of DNase I (NEB; Cat no. M0303S), 10 μL DNase I reaction buffer (NEB; Cat No. B0303S) and 70 μL were added to the mix and incubated at 37° C. for 15 minutes. mRNA was purified with Dynabeads Oligo (dT) 25 (ThermoFisher Scientific; Cat No. 61002) following the manufacturer's instructions. The quality and quantification of the final mRNA produced from linearized amplified hairpin ended DNA was analyzed using an Agilent 4200 TapeStation System and an RNA Assay (Cat Nos. 5067-5576, 5067-5577) following the manufacturer's instructions (FIG. 15B). For functional validations, HEK293T cells were seeded in 96-well plates at a density of 20,000 cells/well in standard growth media supplemented with 10% fetal bovine serum and left to adhere overnight. After 24 hours, media was exchanged, and cells were transfected with the mRNAs. The mRNA was mixed with in Lipofectamine MessengerMAX (ThermoFisher Scientific, Cat No. LMRNA001) following manufacturer's protocol and added to the cells, in triplicates, at 100 ng/well. EGFP expression was monitored over 22 hours post-transfection in an Incucyte SX5 (Sartorius) (FIG. 15C)

### 7.19 Example 19. Generation of Amplified Hairpin-Ended DNA for LV Production

Four plasmid DNA templates were prepared as starting material for the generation of amplified hairpin-ended DNA for lentivirus vector production by quadruple transfection. The first plasmid DNA template comprised between the first and fourth restriction sites for nicking endonuclease, and in the 5′ to 3′ direction of the top strand: a 5′ITR; a spacer; a chimeric 5′ LTR with a CMV-IE promoter, a Psi sequence, a RRE sequence, a cPPT/CTS sequence, an EF-la promoter, an expression cassette encoding anti-CD19 Chimeric Antigen Receptor, a IRES sequence, a nucleotide sequence encoding puromycin resistance, a WPRE sequence, a 3′ LTR, a 3′ UTR and a polyA signal; a spacer; and a 3′ ITR (SEQ ID NO 532). The second plasmid DNA template comprised between the first and fourth restriction sites for nicking endonuclease, and in the 5′ to 3′ direction of the top strand: a 5′ ITR; a spacer; RSV promoter, a 5′ UTR, an expression cassette encoding Rev protein, a 3′ UTR and a polyA signal; a spacer; and a 3′ ITR (SEQ ID NO 533). The third plasmid DNA template comprised between the first and fourth restriction sites for nicking endonuclease, and in the 5′ to 3′ direction of the top strand: a 5′ ITR; a spacer; a CMV-IE promoter, an expression cassette encoding Gag and Pol proteins, a 3′ UTR and a polyA signal; a spacer; and a 3′ ITR (SEQ ID NO 534). The fourth plasmid DNA template comprised between the first and fourth restriction sites for nicking endonuclease, and in the 5′ to 3′ direction of the top strand: a 5′ ITR; a spacer; CMV-IE promoter, an expression cassette encoding VSV-G protein, a 3′ UTR and a polyA signal; a spacer; and a 3′ ITR (SEQ ID NO 535). The initial DNA amplification was performed as described in Example 1 but with a circular DNA templates of between 4.1 kB-11 kB. Digestion of the amplification products, formation of hairpin-ended DNA, and subsequent exonuclease digestion of non-hairpin-ended DNA were performed according to the methods described in Example 1.

TABLE 27
Sequence of Hairpin-ended DNA for Lentiviral Vector Production

| SEQ ID NO | Sequence |
|---|---|
| SEQ ID NO. | AGAAGAGCTCTCTGTACACTCACTCACTCACTGATCCCTGGATACCAAAGGTATCCAGACAC |
| 532 | CCAGTCTTTGACTGGGTGGGATCAGTGAGTGAGTGAGTGTACAGAGAGCTCTTCTGGAGTTA |
| | AGGGCTAGATCTTAGCTTACGTCACTAGAGGGTCCACGTTTAGTTTTTAAGATCCATTGATC |
| | TCCTAAACGCTGCAAGATTCGCAACCTGGTATACTTAGCGCTAGGTCCTAGTGCAGCGGGAC |
| | TTTTTTTCTAAAGTCGTTGAGAGGAGGAGTCGTCAGACCAGATAGCTTTGATGTCCTGATCG |
| | GAAGGATCGTTGGCCCCTTAAGAATACCGATTCTAGGTGCATTGGCGCAGAAAAAAATGCCT |
| | GATGCGACGCTGCGCGTCTTATACTCCCACATATGCCAGATTCAGCAACGGATACGGCTTCC |
| | CCAACTTGCCCACTTCCATACGTGTCCTCCTTACCAGAAATTTATCCTTAAGATCCCGAATC |
| | GTTTAAACGCGATCGCAGTAATCAATTACGGGGTCATTAGTTCATAGCCCATATATGGAGTT |
| | CCGCGTTACATAACTTACGGTAAATGGCCCGCCTGGCTGACCGCCCAACGACCCCCGCCCAT |
| | TGACGTCAATAATGACGTATGTTCCCATAGTAACGCCAATAGGGACTTTCCATTGACGTCAA |
| | TGGGTGGAGTATTTACGGTAAACTGCCCACTTGGCAGTACATCAAGTGTATCATATGCCAAG |
| | TACGCCCCCTATTGACGTCAATGACGGTAAATGGCCCGCCTGGCATTATGCCCAGTACATGA |
| | CCTTATGGGACTTTCCTACTTGGCAGTACATCTACGTATTAGTCATCGCTATTACCATGGTG |
| | ATGCGGTTTTGGCAGTACATCAATGGGCGTGGATAGCGGTTTGACTCACGGGGATTTCCAAG |
| | TCTCCACCCCATTGACGTCAATGGGAGTTTGTTTTGGCACCAAAATCAACGGGACTTTCCAA |
| | AATGTCGTAACAACTCCGCCCCATTGACGCAAATGGGCGGTAGGCGTGTACGGTGGGAGGTC |
| | TATATAAGCAGAGCTCGTTTAGTGAACCGGGGTCTCTCTGGTTAGACCAGATCTGAGCCTGG |
| | GAGCTCTCTGGCTAACTAGGGAACCCACTGCTTAAGCCTCAATAAAGCTTGCCTTGAGTGCT |
| | TCAAGTAGTGTGTGCCCGTCTGTTGTGTGACTCTGGTAACTAGAGATCCCTCAGACCCTTTT |
| | AGTCAGTGTGGAAAATCTCTAGCAGTGGCGCCCGAACAGGGACCTGAAAGCGAAAGGGAAAC |
| | CAGAGCTCTCTCGACGCAGGACTCGGCTTGCTGAAGCGCGCACGGCAAGAGGCGAGGGGCGG |
| | CGACTGGTGAGTACGCCAAAAATTTTGACTAGCGGAGGCTAGAAGGAGAGAGATGGGTGCGA |
| | GAGCGTCAGTATTAAGCGGGGGAGAATTAGATCGCGATGGGAAAAAATTCGGTTAAGGCCAG |
| | GGGGAAAGAAAAAATATAAATTAAAACATATAGTATGGGCAAGCAGGGAGCTAGAACGATTC |
| | GCAGTTAATCCTGGCCTGTTAGAAACATCAGAAGGCTGTAGACAAATACTGGGACAGCTACA |
| | ACCATCCCTTCAGACAGGATCAGAAGAACTTAGATCATTATATAATACAGTAGCAACCCTCT |
| | ATTGTGTGCATCAAAGGATAGAGATAAAAGACACCAAGGAAGCTTTAGACAAGATAGAGGAT |
| | GAGCAAAACAAAAGTAAGACCACCGCACAGCAAGCGGCCGCTGATCTTCAGACCTGGAGGAG |
| | GAGATATGAGGGACAATTGGAGAAGTGAATTATATAAATATAAAGTAGTAAAAATTGAACCA |
| | TTAGGAGTAGCACCCACCAAGGCAAAGAGAAGAGTGGTGCAGAGAGAAAAAAGAGCAGTGGG |
| | AATAGGAGCTTTGTTCCTTGGGTTCTTGGGAGCAGCAGGAAGCACTATGGGCGCAGCCTCAA |
| | TGACGCTGACGGTACAGGCCAGACAATTATTGTCTGGTATAGTGCAGCAGCAGAACAATTTG |
| | CTGAGGGCTATTGAGGCGCAACAGCATCTGTTGCAACTCACAGTCTGGGGCATCAAGCAGCT |
| | CCAGGCAAGAATCCTGGCTGTGGAAAGATACCTAAAGGATCAACAGCTCCTGGGGATTTGGG |
| | GTTGCTCTGGAAAACTCATTTGCACCACTGCTGTGCCTTGGAATGCTAGTTGGAGTAATAAA |
| | TCTCTGGAACAGATTTGGAATCACACGACCTGGATGGAGTGGGACAGAGAAATTAACAATTA |
| | CACAAGCTTAATACACTCCTTAATTGAAGAATCGCAAAACCAGCAAGAAAAGAATGAACAAG |
| | AATTATTGGAATTAGATAAATGGGCAAGTTTGTGGAATTGGTTTAACATAACAAATTGGCTG |
| | TGGTATATAAAATTATTCATAATGATAGTAGGAGGCTTGGTAGGTTTAAGAATAGTTTTTGC |
| | TGTACTTTCTATAGTGAATAGAGTTAGGCAGGGATATTCACCATTATCGTTTCAGACCCACC |
| | TCCCAACCCCGAGGGGACCCGACAGGCCCGAAGGAATAGAAGAAGAAGGTGGAGAGAGAGAC |
| | AGAGACAGATCCATTCGATTAGTGAACGGATCTCGACGGTATCGGTTAACTTTTAAAAGAAA |
| | AGGGGGGATTGGGGGGTACAGTGCAGGGGAAAGAATAGTAGACATAATAGCAACAGACATAC |
| | AAACTAAAGAATTACAAAAACAAATTACAAAAATTCAAAATTTTGGCTCCCGATCGTTGCGT |
| | TACACACACAATTACTGCTGATCGAGTGTAGCCTTCGAATGAAGCGTGAGGCTCCGGTGCCC |
| | GTCAGTGGGCAGAGCGCACATCGCCCACAGTCCCCGAGAAGTTGGGGGGAGGGGTCGGCAAT |
| | TGAACCGGTGCCTAGAGAAGGTGGCGCGGGGTAAACTGGGAAAGTGATGTCGTGTACTGGCT |
| | CCGCCTTTTTCCCGAGGGTGGGGGAGAACCGTATATAAGTGCAGTAGTCGCCGTGAACGTTC |
| | TTTTTCGCAACGGGTTTGCCGCCAGAACACAGGTAAGTGCCGTGTGTGGTTCCCGCGGGCCT |
| | GGCCTCTTTACGGGTTATGGCCCTTGCGTGCCTTGAATTACTTCCACGCCCCTGGCTGCAGT |
| | ACGTGATTCTTGATCCCGAGCTTCGGGTTGGAAGTGGGTGGGAGAGTTCGAGGCCTTGCGCT |
| | TAAGGAGCCCCTTCGCCTCGTGCTTGAGTTGAGGCCTGGCCTGGGCGCTGGGGCCGCCGCGT |
| | GCGAATCTGGTGGCACCTTCGCGCCTGTCTCGCTGCTTTCGATAAGTCTCTAGCCATTTAAA |
| | ATTTTTGATGACCTGCTGCGACGCTTTTTTTCTGGCAAGATAGTCTTGTAAATGCGGGCCAA |
| | GATCTGCACACTGGTATTTCGGTTTTTGGGGCCGCGGGCGGCGACGGGGCCCGTGCGTCCCA |
| | GCGCACATGTTCGGCGAGGCGGGGCCTGCGAGCGCGGCCACCGAGAATCGGACGGGGGTAGT |
| | CTCAAGCTGGCCGGCCTGCTCTGGTGCCTGGCCTCGCGCCGCCGTGTATCGCCCCGCCCTGG |
| | GCGGCAAGGCTGGCCCGGTCGGCACCAGTTGCGTGAGCGGAAAGATGGCCGCTTCCCGGCCC |
| | TGCTGCAGGGAGCTCAAAATGGAGGACGCGGCGCTCGGGAGAGCGGGCGGGTGAGTCACCCA |
| | CACAAAGGAAAAGGGCCTTTCCGTCCTCAGCCGTCGCTTCATGTGACTCCACGGAGTACCGG |
| | GCGCCGTCCAGGCACCTCGATTAGTTCTCGAGCTTTTGGAGTACGTCGTCTTTAGGTTGGGG |
| | GGAGGGGTTTTATGCGATGGAGTTTCCCCACACTGAGTGGGTGGAGACTGAAGTTAGGCCAG |
| | CTTGGCACTTGATGTAATTCTCCTTGGAATTTGCCCTTTTTGAGTTTGGATCTTGGTTCATT |
| | CTCAAGCCTCAGACAGTGGTTCAAAGTTTTTTTCTTCCATTTCAGGTGTCGTGAGCCACCAT |
| | GGCCTTACCAGTGACCGCCTTGCTCCTGCCGCTGGCCTTGCTGCTCCACGCCGCCAGGCCGG |
| | ACATCCAGATGACACAGACTACATCCTCCCTGTCTGCCTCTCTGGGAGACAGAGTCACCATC |
| | AGTTGCAGGGCAAGTCAGGACATTAGTAAATATTTAAATTGGTATCAGCAGAAACCAGATGG |
| | AACTGTTAAACTCCTGATCTACCATACATCAAGATTACACTCAGGAGTCCCATCAAGGTTCA |
| | GTGGCAGTGGGTCTGGAACAGATTATTCTCTCACCATTAGCAACCTGGAGCAAGAAGATATT |
| | GCCACTTACTTTTGCCAACAGGGTAATACGCTTCCGTACACGTTCGGAGGGGGGACCAAGCT |
| | GGAGATCACAGGTGGCGGTGGCTCGGGCGGTGGTGGGTCGGGTGGCGGCGGATCTGAGGTGA |
| | AACTGCAGGAGTCAGGACCTGGCCTGGTGGCGCCCTCACAGAGCCTGTCCGTCACATGCACT |
| | GTCTCAGGGGTCTCATTACCCGACTATGGTGTAAGCTGGATTCGCCAGCCTCCACGAAAGGG |
| | TCTGGAGTGGCTGGGAGTAATATGGGGTAGTGAAACCACATACTATAATTCAGCTCTCAAAT |
| | CCAGACTGACCATCATCAAGGACAACTCCAAGAGCCAAGTTTTCTTAAAAATGAACAGTCTG |
| | CAAACTGATGACACAGCCATTTACTACTGTGCCAAACATTATTACTACGGTGGTAGCTATGC |
| | TATGGACTACTGGGGCCAAGGAACCTCAGTCACCGTGTCCTCAACCACGACGCCAGCGCCGC |
| | GACCACCAACACCGGCGCCCACCATCGCGTCGCAGCCCCTGTCCCTGCGCCCAGAGGCGTGC |
| | CGGCCAGCGGCGGGGGGCGCAGTGCACACGAGGGGGCTGGACTTCGCCTGTGATATCTACAT |
| | CTGGGCGCCCTTGGCCGGGACTTGTGGGGTCCTTCTCCTGTCACTGGTTATCACCCTTTACT |
| | GCAAACGGGGCAGAAAGAAACTCCTGTATATATTCAAACAACCATTTATGAGACCAGTACAA |
| | ACTACTCAAGAGGAAGATGGCTGTAGCTGCCGATTTCCAGAAGAAGAAGAAGGAGGATGTGA |
| | ACTGAGAGTGAAGTTCAGCAGGAGCGCAGACGCCCCCGCGTACAAGCAGGGCCAGAACCAGC |
| | TCTATAACGAGCTCAATCTAGGACGAAGAGAGGAGTACGATGTTTTGGACAAGAGGCGTGGC |
| | CGGGACCCTGAGATGGGGGGAAAGCCGAGAAGGAAGAACCCTCAGGAAGGCCTGTACAATGA |
| | ACTGCAGAAAGATAAGATGGCGGAGGCCTACAGTGAGATTGGGATGAAAGGCGAGCGCCGGA |
| | GGGGCAAGGGGCACGATGGCCTTTACCAGGGTCTCAGTACAGCCACCAAGGACACCTACGAC |
| | GCCCTTCACATGCAGGCCCTGCCCCCTCGCTAGTGAAAGTTGTCTCCTCCTGCACTGACTGA |
| | CTGATACAATGGATATGCCAAAAGCAAAGCGCTATCGCGCCTTACGTTACTGGCCGAAGCCG |
| | CTTGGAATAAGGCCGGTGTGCGTTTGTCTATATGTTATTTTCCACCATATTGCCGTCTTTTG |
| | GCAATGTGAGGGCCCGGAAACCTGGCCCTGTCTTCTTGACGAGCATTCCTAGGGGTCTTTCC |
| | CCTCTCGCCAAAGGAATGCAAGGTCTGTTGAATGTCGTGAAGGAAGCAGTTCCTCTGGAAGC |
| | TTCTTGAAGACAAACAACGTCTGTAGCGACCCTTTGCAGGCAGCGGAACCCCCCACCTGGCG |
| | ACAGGTGCCTCTGCGGCCAAAAGCCACGTGTATAAGATACACCTGCAAAGGCGGCACAACCC |
| | CAGTGCCACGTTGTGAGTTGGATAGTTGTGGAAAGAGTCAAATGGCTCCCCTCAAGCGTATT |
| | CAACAAGGGGCTGAAGGATGCCCAGAAGGTACCCCATTGTATGGGATCTGATCTGGGGCCTC |
| | GGTGCACATGCTTTTCATGTGTTTAGTCGAGGTTAAAAAACGTCTAGGCCCCCCGAACCACG |
| | GGGACGTGGTTTTCCTTTGAAAAACACGATGATAATAACATGACCGAGTACAAGCCCACGGT |
| | GCGCCTCGCCACCCGCGACGACGTCCCCAGGGCCGTACGCACCCTCGCCGCCGCGTTCGCCG |
| | ACTACCCCGCCACGCGCCACACCGTCGATCCGGACCGCCACATCGAGCGGGTCACCGAGCTG |
| | CAAGAACTCTTCCTCACGCGCGTCGGGCTCGACATCGGCAAGGTGTGGGTCGCGGACGACGG |
| | CGCCGCGGTGGCGGTCTGGACCACGCCGGAGAGCGTCGAAGCGGGGGCGGTGTTCGCCGAGA |
| | TCGGCCCGCGCATGGCCGAGTTGAGCGGTTCCCGGCTGGCCGCGCAGCAACAGATGGAGGGC |
| | CTCCTGGCGCCGCACCGGCCCAAGGAGCCCGCGTGGTTCCTGGCCACCGTCGGCGTGTCGCC |
| | CGACCACCAGGGCAAGGGTCTGGGCAGCGCCGTCGTGCTCCCCGGAGTGGAGGCCGCCGAGC |
| | GCGCCGGGGTGCCCGCCTTCCTGGAGACCTCCGCGCCCCGCAACCTCCCCTTCTACGAGCGG |
| | CTCGGCTTCACCGTCACCGCCGACGTCGAGGTGCCCGAAGGACCGCGCACCTGGTGCATGAC |
| | CCGCAAGCCCGGTGCCTAGAACTGCTAGCTTGACTGACTGAGTCGACAATCAACCTCTGGAT |
| | TACAAAATTTGTGAAAGATTGACTGGTATTCTTAACTATGTTGCTCCTTTTACGCTATGTGG |
| | ATACGCTGCTTTAATGCCTTTGTATCATGCTATTGCTTCCCGTATGGCTTTCATTTTCTCCT |
| | CCTTGTATAAATCCTGGTTGCTGTCTCTTTATGAGGAGTTGTGGCCCGTTGTCAGGCAACGT |
| | GGCGTGGTGTGCACTGTGTTTGCTGACGCAACCCCCACTGGTTGGGGCATTGCCACCACCTG |
| | TCAGCTCCTTTCCGGGACTTTCGCTTTCCCCCTCCCTATTGCCACGGCGGAACTCATCGCCG |
| | CCTGCCTTGCCCGCTGCTGGACAGGGGCTCGGCTGTTGGGCACTGACAATTCCGTGGTGTTG |
| | TCGGGGAAGCTGACGTCCTTTCCATGGCTGCTCGCCTGTGTTGCCACCTGGATTCTGCGCGG |
| | GACGTCCTTCTGCTACGTCCCTTCGGCCCTCAATCCAGCGGACCTTCCTTCCCGCGGCCTGC |
| | TGCCGGCTCTGCGGCCTCTTCCGCGTCTTCGCCTTCGCCCTCAGACGAGTCGGATCTCCCTT |
| | TGGGCCGCCTCCCCGCCTGGAATTCGAGCTCGGTACCTTTAAGACCAATGACTTACAAGGCA |
| | GCTGTAGATCTTAGCCACTTTTTAAAAGAAAAGGGGGGACTGGAAGGGCTAATTCACTCCCA |
| | ACGAAGACAAGATCTGCTTTTTGCTTGTACTGGGTCTCTCTGGTTAGACCAGATCTGAGCCT |
| | GGGAGCTCTCTGGCTAACTAGGGAACCCACTGCTTAAGCCTCAATAAAGCTTGCCTTGAGTG |
| | CTTCAAGTAGTGTGTGCCCGTCTGTTGTGTGACTCTGGTAACTAGAGATCCCTCAGACCCTT |
| | TTAGTCAGTGTGGAAAATCTCTAGCAGTCCTGGCCAACGTGAGCACCGTGCTGACCTCCAAA |
| | TATCGTTAAGCTGGAGCCTGGGAGCCGGCCTGGCCCTCCGCCCCCCCCACCCCCGCAGCCCA |
| | CCCCTGGTCTTTGAATAAAGTCTGAGTGAGTGGCCGACAGTGCCCGTGGAGTTCTCGTGACC |
| | TGAGGTGCAGGGCCGGCGCTAGGGACACGTCCGTGCACGTGCCGAGGCCCCCTGTGCAGCTG |
| | CAAGGGACAGGCCTAGCCCTGCAGGCCTAACTCCGCCCATCCCGCCCCTAACTCCGCCCAGT |
| | TCCGCCCATTCTCCGCCTCATGGCTGACTAATTTTTTTTATTTATGCAGAGGCCGAGGCCGC |
| | CTCGGCCTCTGAGCTATTCCAGAAGTAGTGAGGACGCTTTTTTGGAGGCCGAGGCTTTTGCA |
| | AAGATCGAACAAGAGACAGGACCTGCAGGTTAATTAAATTTAAATCATGTGAGCAAAAGGCC |
| | AGCAAAAGGCCAGGAACCGTAAAAAGGCCGCGTTGCTGGCGTTCCGAAAGAACCGGAGATAT |
| | TGGAACGCTCGACGCCGCTACAAGCAGTCGTCTAAGCGCCAAGCTGTCTACCAGGCGGACGC |
| | GCAGTTCGGTCGCGTGAGAAGTACCACAACTGTCTCTATCTTAATCGCAAGTTAACGTGACC |
| | TACTCTAGTCGACGTAGTCAGTAGTCTGCGTGTCACGCGTCAACAAGAAGCCTGACAGCACA |
| | GCCGGCTGTCCGGCACAAGGCACACCAGACTCCGACCTGCTCGCAACAAGTCCACTCGAGTT |
| | GGTCACCAAGGTCGCACTGGCTCTTCTTCTCTGTACACTCACTCACTCACTGATCCCACCCA |
| | GTCAAAGACTGGGTGTCTGGATACCTTTGGTATCCAGGGATCAGTGAGTGAGTGAGTGTACA |
| | GAGA |
| SEQ ID NO. | AGAAGAGCTCTCTGTACACTCACTCACTCACTGATCCCTGGATACCAAAGGTATCCAGACAC |
| 533 | CCAGTCTTTGACTGGGTGGGATCAGTGAGTGAGTGAGTGTACAGAGAGCTCTTCTGGAGGGA |
| | GTGGCCAAAACCAACTAGACAACTTTGTATATGAATTACGAATTCGATGTACGGGCCAGATA |
| | TACGCGTATCTGAGGGGACTAGGGTGTGTTTAGGCGAAAAGCGGGGCTTCGGTTGTACGCGG |
| | TTAGGAGTCCCCTCAGGATATAGTAGTTTCGCTTTTGCATAGGGAGGGGGAAATGTAGTCTT |
| | ATGCAATACTCTTGTAGTCTTGCAACATGGTAACGATGAGTTAGCAACATGCCTTACAAGGA |
| | GAGAAAAAGCACCGTGCATGCCGATTGGTGGAAGTAAGGTGGTACGATCGTGCCTTATTAGG |
| | AAGGCAACAGACGGGTCTGACATGGATTGGACGAACCACTGAATTCCGCATTGCAGAGATAT |
| | TGTATTTAAGTGCCTAGCTCGATACAATAAACGCCATTTGACCATTCACCACATTGGTGTGC |
| | ACCTCCAAGCTCGAGCTCGTTTAGTGAACCGTCAGATCGCCTGGAGATGCCATCCACGCTGT |
| | TTTGACCTCCATAGAAGACACCGGGACCGATCCAGCCTCCCCTCGAAGCTAGTCGATTAGGC |
| | ATCTCCTATGGCAGGAAGAAGCGGAGACAGCGACGAAGACCTCCTCAAGGCAGTCAGACTCA |
| | TCAAGTTTCTCTATCAAAGCAACCCACCTCCCAATCCCGAGGGGACCCGACAGGCCCGAAGG |
| | AATAGAAGAAGAAGGTGGAGAGAGAGACAGAGACAGATCCATTCGATTAGTGAACGGATCCT |
| | TAGCACTTATCTGGGACGATCTGCGGAGCCTGTGCCTCTTCAGCTACCACCGCTTGAGAGAC |
| | TTACTCTTGATTGTAACGAGGATTGTGGAACTTCTGGGACGCAGGGGGTGGGAAGCCCTCAA |
| | ATATTGGTGGAATCTCCTACAATATTGGAGTCAGGAGCTAAAGAATAGTGCTGTTAGCTTGC |
| | TCAATGCCACAGCTATAGCAGTAGCTGAGGGGACAGATAGGGTTATAGAAGTAGTACAAGAA |
| | GCTTGGCACTGGCCGTCGTTTTACAACGTCGTGATCTGAGCCTGGGAGATCTCTGGCTAACT |
| | AGGGAACCCACTGCTTAAGCCTCAATAAAGCTTGCCTTGAGTGCTTCAAGTAGTGTGTGCCC |
| | GTCTGTTGTGTGACTCTGGTAACTAGAGATCAGGAAAACCCTGGCGTTACCCAACTTAATCG |
| | CCTTGCAGCACATCCCCCTTTCGCCAGCTGGCGTAATAGCGAAGAGGCCCGCACCGATCGCC |
| | CTTCCCAACAGTTGCGCAGCCTGAATGGCGAATGGCGCCTGATGCGGTATTTTCTCCTTACG |
| | CATCTGTGCGGTATTTCACACCGCATACGTCAAAGCAACCATAGAACTTCAGGAACCCCTTT |
| | TAAGAGTAACATAACTGCTGGAGTAGATGAAGATGGAGGGCCACTTTACTGGCTCTTCTTCT |
| | CTGTACACTCACTCACTCACTGATCCCACCCAGTCAAAGACTGGGTGTCTGGATACCTTTGG |
| | TATCCAGGGATCAGTGAGTGAGTGAGTGTACAGAGA |
| SEQ ID NO. | AGAAGAGCTCTCTGTACACTCACTCACTCACTGATCCCTGGATACCAAAGGTATCCAGACAC |
| 534 | CCAGTCTTTGACTGGGTGGGATCAGTGAGTGAGTGAGTGTACAGAGAGCTCTTCTGGAGGGA |
| | GTGGCCAAAACCAACTAGACAACTTTGTATATGAATCTGCGGCCGCACTAGTGCTAGACTGC |
| | CATGTCGAGGGATTCCGGGTCACTGTGAGTGGGGGAGGCAGGGAAGAAGGGCTCACAGGACA |
| | GTCAAACCATGCCCCCTGTTTTTCCTTCTTCAAGTAGACCTCTATAAGACAACAGAGACAAC |
| | TAAGGCTGAGTGGCCAGGCGAGGAGAAACCATCTCGCCGTAAAACATGGAAGGAACACTTCA |
| | GGGGAAAGGTGGTATCTCTAAGCAAGAGAACTGAGTGGAGTCAAGGCTGAGAGATGCAGGAT |
| | AAGCAAATGGGTAGTGAAAAGACATTCATGAGGACAGCTAAAACAATAAGTAATGTAAAATA |
| | CAGCATAGCAAAACTTTAACCTCCAAATCAAGCCTCTACTTGAATCCTTTTCTGAGGGATGA |
| | ATAAGGCATAGGCATCAGGGGCTGTTGCCAATGTGCATTAGCTGTTTGCAGCCTCACCTTCT |
| | TTCATGGAGTTTAAGATATAGTGTATTTTCCCAAGGTTTGAACTAGCTCATCATTTCTTTAT |
| | GTTTTAAATGCACTGACCTCCCACATTCCCTTTTTAGTAAAATATTCAGAAATAATTTAAAT |
| | ACATCATTGCAATGAAAATAAATGTTTTTTATTAGGCAGAATCCAGATGCTCAAGGCCCTTC |
| | ATAATATCCCCCAGTTTAGTAGTTGGACTTAGGGAACAAAGGAACCTTTAATAGAAATTGGA |
| | CAGCAAGAAAGCGAGCTTAGTGATACTTGTGGGCCAGGGCATTAGCCACACCAGCCACCACT |
| | TTCTGATAGGCAGCCTGCACTGGTGGGGTGAATTCCGCGGAAGCTTGTGTAATTGTTAATTT |
| | CTCTGTCCCACTCCATCCAGGTCGTGTGATTCCAAATCTGTTCCAGAGATTTATTACTCCAA |
| | CTAGCATTCCAAGGCACAGCAGTGGTGCAAATGAGTTTTCCAGAGCAACCCCAAATCCCCAG |
| | GAGCTGTTGATCCTTTAGGTATCTTTCCACAGCCAGGATTCTTGCCTGGAGCTGCTTGATGC |
| | CCCAGACTGTGAGTTGCAACAGATGCTGTTGCGCCTCAATAGCCCTCAGCAAATTGTTCTGC |
| | TGCTGCACTATACCAGACAATAATTGTCTGGCCTGTACCGTCAGCGTCATTGACGCTGCGCC |
| | CATAGTGCTTCCTGCTGCTCCCAAGAACCCAAGGAACAAAGCTCCTGCGGCCGCTCCGGAAT |
| | TCCATGTGTTAATCCTCATCCTGTCTACTTGCCACACAATCATCACCTGCCATCTGTTTTCC |
| | ATAATCCCTGATGATCTTTGCTTTTCTTCTTGGCACTACTTTTATGTCACTATTATCTTGTA |
| | TTACTACTGCCCCTTCACCTTTCCAGAGGAGCTTTGCTGGTCCTTTCCAAACTGGATCTCTG |
| | CTGTCCCTGTAATAAACCCGAAAATTTTGAATTTTTGTAATTTGTTTTTGTAATTCTTTAGT |
| | TTGTATGTCTGTTGCTATTATGTCTACTATTCTTTCCCCTGCACTGTACCCCCCAATCCCCC |
| | CTTTTCTTTTAAAATTGTGGATGAATACTGCCATTTGTACTGCTGTCTTAAGATGTTCAGCC |
| | TGATCTCTTACCTGTCCTATAATTTTCTTTAATTCTTTATTCATAGATTCTATTACTCCTTG |
| | ACTTTGGGGATTGTAGGGAATGCCAAATTCCTGCTTGATCCCCGCCCACCAACAGGCGGCCT |
| | TAACTGTAGTACTGGTGAAATTGCTGCCATTGTCTGTATGTACTGTTTTTACTGGCCATCTT |
| | CCTGCTAATTTTAAGAGGAAGTATGCTGTTTCTTGCCCTGTCTCTGCTGGAATTACTTCTGC |
| | TTCTATATATCCACTGGCTACATGAACTGCTACCAAGATAACTTTTCCTTCTAAATGTGTAC |
| | AATCTAGCTGCCATATTCCTGGGCTACAGTCTACTTGTCCATGCATGGCTTCCCCTTTTAGC |
| | TGACATTTATCACAGCTGGCTACTATTTCTTTTGCTACTACAGGTGGTAGGTTAAAATCACT |
| | AGCCATTGCTCTCCAATTACTGTGATATTTCTCATGTTCTTCTTGGGCCTTATCTATTCCAT |
| | CTAAAAATAGTACTTTCCTGATTCCAGCACTGACCAATTTATCTACTTGTTCATTTCCTCCA |
| | ATTCCTTTGTGTGCTGGCACCCATGCCAGGTAGACTTTTTCCTTTTTTATTAACTGCTCTAT |
| | TATTTGACTGACTAACTCTGATTCACTCTTATCTGGTTGTGCTTGAATGATTCCCAATGCAT |
| | ATTGTGAGTCTGTCACTATGTTTACTTCTAATCCCGAATCCTGCAAAGCTAGATGAATTGCT |
| | TGTAACTCAGTCTTCTGATTTGTTGTGTCCGTTAGGGGGACAACTTTTTGTCTTCCTCTGTC |
| | AGTTACATATCCTGCTTTTCCTAATTTAGTTTCCCTATTGGCTGCCCCATCTACATAGAAAG |
| | TTTCTGCTCCTATTATGGGTTCTTTCTCTAACTGATACCATAACTTCACTAAGGGAGGGGTA |
| | TTGACAAACTCCCACTCAGGAATCCAGGTGGCTTGCCAATACTCTGTCCACCATGCTTCCCA |
| | TGTTTCCTTTTGTATGGGTAATTTAAATTTAGGAGTCTTTCCCCATATTACTATGCTTTCTG |
| | TGGCTATTTTTTGTACTGCCTCTGTTAATTGTTTCACATCATTAGTGTGGGCACCCTTCATT |
| | CTTGCATACTTTCCTGTTTTCAGATTTTTAAATGGCTCTTGATAAATTTGATATGTCCATTG |
| | GCCTTGCCCCTGCTTCTGTATTTCTGCTATTAAGTCTTTTGATGGGTCATAATACACTCCAT |
| | GTACCGGTTCTTTTAGAATCTCCCTGTTTTCTGCCAGTTCTAGCTCTGCTTCTTCTGTTAGT |
| | GGTACTACTTCTGTTAGTGCTTTGGTTCCCCTAAGAAGTTTACATAATTGCCTTACTTTAAT |
| | CCCTGCATAAATCTGACTTGCCCAATTCAATTTTCCCACTAATTTCTGTATGTCATTGACAG |
| | TCCAGCTGTCCTTTTCTGGCAGCACTATAGGCTGTACTGTCCATTTATCAGGATGGAGTTCA |
| | TAACCCATCCAAAGGAATGGAGGTTCTTTCTGATGTTTTTTGTCTGGTGTGGTAAATCCCCA |
| | CCTCAACAGATGTTGTCTCAGTTCCTCTATTTTTGTTCTATGCTGCCCTATTTCTAAGTCAG |
| | ATCCTACATACAAATCATCCATGTATTGATAGATGACTATGTCTGGATTTTGTTTTCTAAAA |
| | GGCTCTAAGATTTTTGTCATGCTACACTGGAATATTGCTGGTGATCCTTTCCATCCCTGTGG |
| | AAGCACATTGTACTGATATCTAATCCCTGGTGTCTCATTGTTTATACTAGGTATGGTAAATG |
| | CAGTATACTTCCTGAAGTCTTTATCTAAGGGAACTGAAAAATATGCATCGCCCACATCCAGT |
| | ACTGTTACTGATTTTTTCTGTTTTAACCCTGCAGGATGTGGTATTCCTAATTGAACTTCCCA |
| | GAAATCTTGAGTTCTCTTATTAAGTTCTCTGAAATCTACTAATTTTCTCCATTTAGTACTGT |
| | CTTTTTTCTTTATGGCAAATACTGGAGTATTGTATGGATTTTCAGGCCCAATTTTTGAAATT |
| | TTTCCTTCCTTTTCCATTTCTGTACAAATTTCTACTAATGCTTTTATTTTTTCTTCTGTCAA |
| | TGGCCATTGTTTAACTTTTGGGCCATCCATTCCTGGCTTTAATTTTACTGGTACAGTCTCAA |
| | TAGGACTAATGGGAAAATTTAAAGTGCAGCCAATCTGAGTCAACAGATTTCTTCCAATTATG |
| | TTGACAGGTGTAGGTCCTACTAATACTGTACCTATAGCTTTATGTCCGCAGATTTCTATGAG |
| | TATCTGATCATACTGTCTTACTTTGATAAAACCTCCAATTCCCCCTATCATTTTTGGTTTCC |
| | ATCTTCCTGGCAAATTCATTTCTTCTAATACTGTATCATCTGCTCCTGTATCTAATAGAGCT |
| | TCCTTTAATTGCCCCCCTATCTTTATTGTGACGAGGGGTCGCTGCCAAAGAGTGATCTGAGG |
| | GAAGCTAAAGGATACAGTTCCTTGTCTATCGGCTCCTGCTTCTGAGAGGGAGTTGTTGTCTC |
| | TTCCCCAAACCTGAAGCTCTCTTCTGGTGGGGCTGTTGGCTCTGGTCTGCTCTGAAGAAAAT |
| | TCCCTGGCCTTCCCTTGTGGGAAGGCCAGATCTTCCCTAAAAAATTAGCCTGTCTCTCAGTA |
| | CAATCTTTCATTTGGTGTCCTTCCTTTCCACATTTCCAACAGCCCTTTTTCCTAGGGGCCCT |
| | GCAATTTTTGGCTATGTGCCCTTCTTTGCCACAATTGAAACACTTAACAGTCTTTCTTTGGT |
| | TCCTAAAATTGCCTTTCTGTATCATTATGGTAGCTGGATTTGTTACTTGGCTCATTGCTTCA |
| | GCCAAAACTCTTGCTTTATGGCCGGGTCCCCCCACTCCCTGACATGCTGTCATCATTTCTTC |
| | TAGTGTCGCTCCTGGTCCCAATGCTTTTAAAATAGTCTTACAATCTGGGTTCGCATTTTGGA |
| | CCAACAAGGTTTCTGTCATCCAATTTTTTACCTCTTGTGAAGCTTGCTCGGCTCTTAGAGTT |
| | TTATAGAATCGGTCTACATAGTCTCTAAAGGGTTCCTTTGGTCCTTGTCTTATGTCCAGAAT |
| | GCTGGTAGGGCTATACATTCTTACTATTTTATTTAATCCCAGGATTATCCATCTTTTATAGA |
| | TTTCTCCTACTGGGATAGGTGGATTATGTGTCATCCATCCTATTTGTTCCTGAAGGGTACTA |
| | GTAGTTCCTGCTATGTCACTTCCCCTTGGTTCTCTCATCTGGCCTGGTGCAATAGGCCCTGC |
| | ATGCACTGGATGCACTCTATCCCATTCTGCAGCTTCCTCATTGATGGTCTCTTTTAACATTT |
| | GCATGGCTGCTTGATGTCCCCCCACTGTGTTTAGCATGGTGTTTAAATCTTGTGGGGTGGCT |
| | CCTTCTGATAATGCTGAAAACATGGGTATCACTTCTGGGCTGAAAGCCTTCTCTTCTACTAC |
| | TTTTACCCATGCATTTAAAGTTCTAGGTGATATGGCCTGATGTACCATTTGCCCCTGGATGT |
| | TCTGCACTATAGGGTAATTTTGGCTGACCTGATTGCTGTGTCCTGTGTCAGCTGCTGCTTGC |
| | TGTGCTTTTTTCTTACTTTTGTTTTGCTCCTCCTCTATCTTGTCTAAAGCTTCCTTGGTGTC |
| | TTTTATCTCTATCCTTTGATGCACACAATAGAGGGTTGCTACTGTATTATATAATGATCTAA |
| | GTTCTTCTGATCCTGTCTGAAGGGATGGTTGTAGCTGTCCCAGTATTTGTCTACAGCCTTCT |
| | GATGTTTCTAACAGGCCAGGATTAACTGCGAATCGTTCTAGCTCCCTGCTTGCCCATACTAT |
| | ATGTTTTAATTTATATTTTTTCTTTCCCCCTGGCCTTAACCGAATTTTTTCCCAGCGATCTA |
| | ATTCTCCCCCGCTTAATACTGACGCTCTCGCACCCATGGCGGCGGCAGATCTCGAATTCAGA |
| | TCTCACGTGCTTTGCCAAAGTGATGGGCCAGCACACAGACCAGCACGTTGCCCAGGAGCTGT |
| | GGGAGGAAGATAAGAGGTATGAACATGATTAGCAAAAGGGCCTAGCTTGGACTCAGAATAAT |
| | CCAGCCTTATCCCAACCATAAAATAAAAGCAGAATGGTAGCTGGATTGTAGCTGCTATTAGC |
| | AATATGAAACCTCTTACATCAGTTACAATTTATATGCAGAAATATTTATATGCAGAAATATT |
| | GCTATTGCCTTAACCCAGAAATTATCACTGTTATTCTTTAGAATGGTGCAAAGAGGCATGAT |
| | ACATTGTATCATTATTGCCCTGAAAGAAAGAGATTAGGGAAAGTATTAGAAATAAGATAAAC |
| | AAAAAAGTATATTAAAAGAAGAAAGCATTTTTTAAAATTACAAATGCAAAATTACCCTGATT |
| | TGGTCAATATGTGTACCCTGTTACTTCTCCCCTTCCTATGACATGAACTTAACCATAGAAAA |
| | GAAGGGGAAAGAAAACATCAAGGGTCCCATAGACTCACCCTGAAGTTCTCAGGATCCGAGCT |
| | CGGGACCACATGTAAGCTTCGAGGGGAGGCTGGATCGGTCCCGGTGTCTTCTATGGAGGTCA |
| | AAACAGCGTGGATGGCATCTCCAGGCGATCTGACGGTTCACTAAACGAGCTCTGCTTATATA |
| | GACCTCCCACCGTACACGCCTACCGCCCATTTGCGTCAATGGGGCGGAGTTGTTACGACATT |
| | TTGGAAAGTCCCGTTGATTTTGGTGCCAAAACAAACTCCCATTGACGTCAATGGGGTGGAGA |
| | CTTGGAAATCCCCGTGAGTCAAACCGCTATCCACGCCCATTGATGTACTGCCAAAACCGCAT |
| | CACCATGGTAATAGCGATGACTAATACGTAGATGTACTGCCAAGTAGGAAAGTCCCATAAGG |
| | TCATGTACTGGGCATAATGCCAGGCGGGCCATTTACCGTCATTGACGTCAATAGGGGGCGTA |
| | CTTGGCATATGATACACTTGATGTACTGCCAAGTGGGCAGTTTACCGTAAATACTCCACCCA |
| | TTGACGTCAATGGAAAGTCCCTATTGGCGTTACTATGGGAACATACGTCATTATTGACGTCA |
| | ATGGGGGGGGGTCGTTGGGCGGTCAGCCAGGCGGGCCATTTACCGTAAGTTATGTAACGCGG |
| | AACTCCATATATGGGCTATGAACTAATGACCCCGTAATTGATTACTATTAATAACTAGTCAA |
| | TAATCAATGTCAACATGGCGGTAATGTTGGACATGAGCCAATATAAATGTACATATTATGAT |
| | ATGGATACAACGTATGCAATAACTTCAGGAACCCCTTTTAAGAGTAACATAACTGCTGGAGT |
| | AGATGAAGATGGAGGGCCACTTTACTGGCTCTTCTTCTCTGTACACTCACTCACTCACTGAT |
| | CCCACCCAGTCAAAGACTGGGTGTCTGGATACCTTTGGTATCCAGGGATCAGTGAGTGAGTG |
| | AGTGTACAGAGA |
| SEQ ID NO. | AGAAGAGCTCTCTGTACACTCACTCACTCACTGATCCCTGGATACCAAAGGTATCCAGACAC |
| 535 | CCAGTCTTTGACTGGGTGGGATCAGTGAGTGAGTGAGTGTACAGAGAGCTCTTCTGGAGGGA |
| | GTGGCCAAAACCAACTAGACAACTTTGTATATGAAGCCCATTGCATACGTTGTATCCATATC |
| | ATAATATGTACATTTATATTGGCTCATGTCCAACATTACCGCCATGTTGACATTGATTATTG |
| | ACTAGTTATTAATAGTAATCAATTACGGGGTCATTAGTTCATAGCCCATATATGGAGTTCCG |
| | CGTTACATAACTTACGGTAAATGGCCCGCCTGGCTGACCGCCCAACGACCCCCGCCCATTGA |
| | CGTCAATAATGACGTATGTTCCCATAGTAACGCCAATAGGGACTTTCCATTGACGTCAATGG |
| | GTGGAGTATTTACGGTAAACTGCCCACTTGGCAGTACATCAAGTGTATCATATGCCAAGTAC |
| | GCCCCCTATTGACGTCAATGACGGTAAATGGCCCGCCTGGCATTATGCCCAGTACATGACCT |
| | TATGGGACTTTCCTACTTGGCAGTACATCTACGTATTAGTCATCGCTATTACCATGGTGATG |
| | CGGTTTTGGCAGTACATCAATGGGCGTGGATAGCGGTTTGACTCACGGGGATTTCCAAGTCT |
| | CCACCCCATTGACGTCAATGGGAGTTTGTTTTGGCACCAAAATCAACGGGACTTTCCAAAAT |
| | GTCGTAACAACTCCGCCCCATTGACGCAAATGGGCGGTAGGCGTGTACGGTGGGAGGTCTAT |
| | ATAAGCAGAGCTCGTTTAGTGAACCGTCAGATCGCCTGGAGATGCCATCCACGCTGTTTTGA |
| | CCTCCATAGAAGACACCGGGACCGATCCAGCCTCCGGTCGACCGATCCTGAGAACTTCAGGG |
| | TGAGTTTGGGGACCCTTGATTGTTCTTTCTTTTTCGCTATTGTAAAATTCATGTTATATGGA |
| | GGGGGCAAAGTTTTCAGGGTGTTGTTTAGAATGGGAAGATGTCCCTTGTATCACCATGGACC |
| | CTCATGATAATTTTGTTTCTTTCACTTTCTACTCTGTTGACAACCATTGTCTCCTCTTATTT |
| | TCTTTTCATTTTCTGTAACTTTTTCGTTAAACTTTAGCTTGCATTTGTAACGAATTTTTAAA |
| | TTCACTTTTGTTTATTTGTCAGATTGTAAGTACTTTCTCTAATCACTTTTTTTTCAAGGCAA |
| | TCAGGGTATATTATATTGTACTTCAGCACAGTTTTAGAGAACAATTGTTATAATTAAATGAT |
| | AAGGTAGAATATTTCTGCATATAAATTCTGGCTGGCGTGGAAATATTCTTATTGGTAGAAAC |
| | AACTACACCCTGGTCATCATCCTGCCTTTCTCTTTATGGTTACAATGATATACACTGTTTGA |
| | GATGAGGATAAAATACTCTGAGTCCAAACCGGGCCCCTCTGCTAACCATGTTCATGCCTTCT |
| | TCTCTTTCCTACAGCTCCTGGGCAACGTGCTGGTTGTTGTGCTGTCTCATCATTTTGGCAAA |
| | GAATTCCTCGACGGATCCCTCGAGGAATTCTGACACTATGAAGTGCCTTTTGTACTTAGCCT |
| | TTTTATTCATTGGGGTGAATTGCAAGTTCACCATAGTTTTTCCACACAACCAAAAAGGAAAC |
| | TGGAAAAATGTTCCTTCTAATTACCATTATTGCCCGTCAAGCTCAGATTTAAATTGGCATAA |
| | TGACTTAATAGGCACAGCCTTACAAGTCAAAATGCCCAAGAGTCACAAGGCTATTCAAGCAG |
| | ACGGTTGGATGTGTCATGCTTCCAAATGGGTCACTACTTGTGATTTCCGCTGGTATGGACCG |
| | AAGTATATAACACATTCCATCCGATCCTTCACTCCATCTGTAGAACAATGCAAGGAAAGCAT |
| | TGAACAAACGAAACAAGGAACTTGGCTGAATCCAGGCTTCCCTCCTCAAAGTTGTGGATATG |
| | CAACTGTGACGGATGCCGAAGCAGTGATTGTCCAGGTGACTCCTCACCATGTGCTGGTTGAT |
| | GAATACACAGGAGAATGGGTTGATTCACAGTTCATCAACGGAAAATGCAGCAATTACATATG |
| | CCCCACTGTCCATAACTCTACAACCTGGCATTCTGACTATAAGGTCAAAGGGCTATGTGATT |
| | CTAACCTCATTTCCATGGACATCACCTTCTTCTCAGAGGACGGAGAGCTATCATCCCTGGGA |
| | AAGGAGGGCACAGGGTTCAGAAGTAACTACTTTGCTTATGAAACTGGAGGCAAGGCCTGCAA |
| | AATGCAATACTGCAAGCATTGGGGAGTCAGACTCCCATCAGGTGTCTGGTTCGAGATGGCTG |
| | ATAAGGATCTCTTTGCTGCAGCCAGATTCCCTGAATGCCCAGAAGGGTCAAGTATCTCTGCT |
| | CCATCTCAGACCTCAGTGGATGTAAGTCTAATTCAGGACGTTGAGAGGATCTTGGATTATTC |
| | CCTCTGCCAAGAAACCTGGAGCAAAATCAGAGCGGGTCTTCCAATCTCTCCAGTGGATCTCA |
| | GCTATCTTGCTCCTAAAAACCCAGGAACCGGTCCTGCTTTCACCATAATCAATGGCACCCTA |
| | AAATACTTTGAGACCAGATACATCAGAGTCGATATTGCTGCTCCAATCCTCTCAAGAATGGT |
| | CGGAATGATCAGTGGAACTACCACAGAAAGGGAACTGTGGGATGACTGGGCACCATATGAAG |
| | ACGTGGAAATTGGACCCAATGGAGTTCTGAGGACCAGTTCAGGATATAAGTTTCCTTTATAC |
| | ATGATTGGACATGGTATGTTGGACTCCGATCTTCATCTTAGCTCAAAGGCTCAGGTGTTCGA |
| | ACATCCTCACATTCAAGACGCTGCTTCGCAACTTCCTGATGATGAGAGTTTATTTTTTGGTG |
| | ATACTGGGCTATCCAAAAATCCAATCGAGCTTGTAGAAGGTTGGTTCAGTAGTTGGAAAAGC |
| | TCTATTGCCTCTTTTTTCTTTATCATAGGGTTAATCATTGGACTATTCTTGGTTCTCCGAGT |
| | TGGTATCCATCTTTGCATTAAATTAAAGCACACCAAGAAAAGACAGATTTATACAGACATAG |
| | AGATGAACCGACTTGGAAAGTAACTCAAATCCTGCACAACAGATTCTTCATGTTTGGACCAA |
| | ATCAACTTGTGATACCATGCTCAAAGAGGCCTCAATTATATTTGAGTTTTTAATTTTTATGA |
| | AAAAAAAAAAAAAAAACGGAATTCCTCGAGGGATCCGTCGAGGAATTCACTCCTCAGGTGCA |
| | GGCTGCCTATCAGAAGGTGGTGGCTGGTGTGGCCAATGCCCTGGCTCACAAATACCACTGAG |
| | ATCTTTTTCCCTCTGCCAAAAATTATGGGGACATCATGAAGCCCCTTGAGCATCTGACTTCT |
| | GGCTAATAAAGGAAATTTATTTTCATTGCAATAGTGTGTTGGAATTTTTTGTGTCTCTCACT |
| | CGGAAGGACATATGGGAGGGCAAATCATTTAAAACATCAGAATGAGTATTTGGTTTAGAGTT |
| | TGGCAACATATGCCCATATGCTGGCTGCCATGAACAAAGGTTGGCTATAAAGAGGTCATCAG |
| | TATATGAAACAGCCCCCTGCTGTCCATTCCTTATTCCATAGAAAAGCCTTGACTTGAGGTTA |
| | GATTTTTTTTATATTTTGTTTTGTGTTATTTTTTTCTTTAACATCCCTAAAATTTTCCTTAC |
| | ATGTTTTACTAGCCAGATTTTTCCTCCTCTCCTGACTACTCCCAGTCATAGCTGTCCCTCTT |
| | CTCTTATGGAGATCCCTCGACGGATCGGCCGCAATTCGTAATCATAACTTCAGGAACCCCTT |
| | TTAAGAGTAACATAACTGCTGGAGTAGATGAAGATGGAGGGCCACTTTACTGGCTCTTCTTC |
| | TCTGTACACTCACTCACTCACTGATCCCACCCAGTCAAAGACTGGGTGTCTGGATACCTTTG |
| | GTATCCAGGGATCAGTGAGTGAGTGAGTGTACAGAGA |

### 7.20 Example 20. Lentiviral Vector Production from Hairpin-Ended DNA Molecules

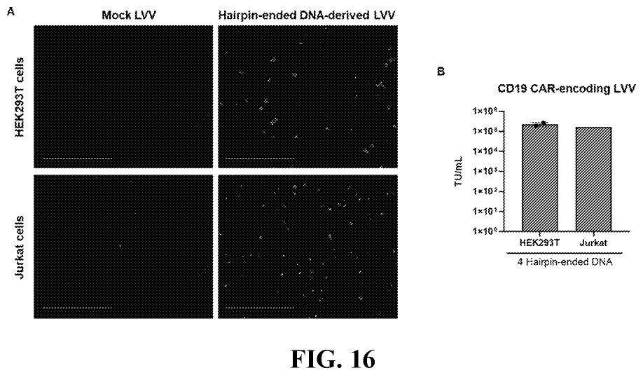

Lentiviral vector production was performed in HEK293T cells by transfection of hairpin-ended DNA. Cells were seeded at a density of 120,000 cells/cm² in standard growth media supplemented with 10% fetal bovine serum. Next day, a quadruple transfection of the cells was performed by formulating the four plasmid DNA templates produced in Example 19 (SEQ ID NOs. 532. 533, 534, and 535) in jetPrime transfection reagent (PolyPlus, Cat. No. 101000046), at a ratio of 3:2:1:1, for a final amount of 2 μg DNA per million cells. Media of transfected cells was changed at 5 h post-transfection to serum-free Opti-MEM media (ThermoFisher Scientific, Cat. No. 51985-026). At 48 h post-transfection, lentiviral vector conditioned media was harvested and clarified by low-speed centrifugation, followed by supernatant collection. Clarified lentiviral vector conditioned media was serially diluted in Opti-MEM, and HEK293T cells were reverse transduced with each viral dilution prepared at a ratio of 20 μL per 20,000 cells, whereas Jurkat cells were transduced at a ratio of 20 μL per 30,000 cells, in a final volume of 120 μL. At 72 h post-transduction, transduced cells were harvested, washed with Dulbecco's phosphate buffered saline, incubated for 1 h with recombinant CD19-PE (ACRO Biosystems, Cat. No. CD9-HP2H3) and washed for removal of unbound recombinant protein. Stained cells were imaged on Incucyte SX5 to verify CD19-PE binding to the surface of the transduced cells. Stained cells were also analysed by flow cytometry and the percentage of PE-positive cells was determined, in order to calculate the infectious viral titer as the concentration of TU/mL. Produced CD19 CAR lentiviral vectors were functional, effectively transducing both HEK293T and Jurkat cells (FIG. 16.A) at an infectious titer of 2.05×105±4.32×104 TU/mL (FIG. 16.B).

### 7.21 Example 21. ClaI Digestion of RCA and not Digestion on Plasmid

Methylation-sensitive restriction enzymes (MSRE) can be blocked by bacterial DNA methylation and therefore cannot cut methylated circular DNA templates originating from bacterial cells, which can used for DNA amplification and production of hairpin-ended DNA molecules as described herein. Without wishing to be bound by theory, MSREs are not able to cut DNA resulting from an enzymatic DNA amplification process. This fact can be used to digest amplified DNA resulting from a DNA amplification process described herein in situ without inhibiting amplification of the circular DNA template.

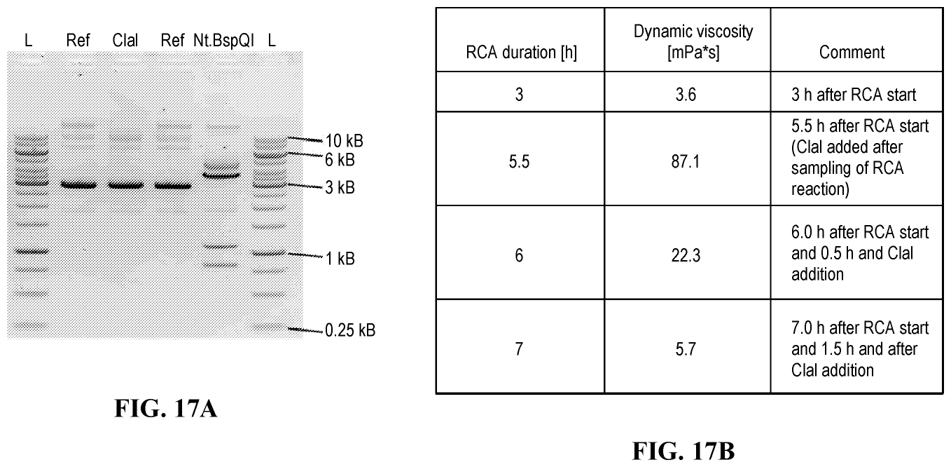

Preparation of circular template DNA: A nucleic acid sequence, encoding an expression cassette flanked by a 3′ ITR and a 5′ ITR was designed in silico and cloned into a pUC57 backbone containing additional ClaI (a MSRE) cutting sites that undergo methylation within the bacterial cell. Single and double nicking sites for Nt.BspQI, which facilitate later formation of hairpin ended DNA molecules from amplified DNA were also present on the circular DNA construct. The final ~5 kilobase (kb) circular template DNA was transformed and amplified overnight in a NEB stable Escherichia coli strain followed by plasmid isolation using a commercial plasmid isolation kit (Nucleobond Xtra Maxi Plus EF (Macherey Nagel)). The isolated plasmid was dissolved in 5 mM TRIS, 0.1 mM EDTA, pH 8.0. Digestion check: The isolated plasmid was digested selectively with 0.1 U/uL ClaI (New England Biolabs; Cat. No R0197S) or with 0.1 U/uL Nt.BspQI (New England Biolabs; Cat. No R0644S) at 37° C. for 1 h. Digests and non-treated plasmid references were mixed with 6×DNA loading dye containing EDTA to stop the reaction and run on an 1% agarose gel. Due to methylation of the ClaI cutting sites, the ClaI exposed plasmid did not show digestion by ClaI. When digested with Nt.BspQI, however, the plasmid clearly shows digestion compared to the non-treated plasmid sample (FIG. 17A).

MSRE within the DNA amplification process: DNA amplification of the plasmid described in this example was performed according to the methods detailed in Example 1. Samples were collected after 3 and 5.5 hrs of amplification, after addition of 0.1 U/uL ClaI enzyme, and after incubation with ClaI for 0.5 h and 1.5 hrs (=6 or 7 hr of total amplification reaction, respectively). After collection, each sample was mixed with EDTA to stop the reaction and the dynamic viscosity of the sample was measured at 20° C. with a Lovis 2000 M/ME viscometer (Anton Paar GmbH). FIG. 17B shows the corresponding dynamic viscosity of the samples. It is shown that addition of ClaI to the DNA amplification reaction decreases the dynamic viscosity of the reaction over time.

What is claimed is:

1. A method for preparing hairpin-ended DNA molecules, wherein the method comprises:
a. providing a circular DNA molecule as a template;
b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand;
c. incubating the amplification product with one or more nicking endonucleases recognizing the four restriction sites;
d. denaturing and thereby creating a DNA fragment that comprises the two single strand DNA overhangs as specified in step b upon separation of the top from the bottom strand; and
e. annealing the single strand DNA overhangs and thereby creating a hairpinned inverted repeat on each end of the DNA fragment resulting from the denaturing step to produce the hairpin-ended DNA molecule.

2. A method for amplifying precursors of hairpin-ended DNA molecules, wherein the method comprises:
a. providing a circular DNA molecule as a template; and
b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand.

3. The method of claim 2, further comprising
a. incubating the amplification product with one or more nicking endonucleases recognizing the four restriction sites;
b. denaturing and thereby creating a DNA fragment that comprises the two single strand DNA overhangs as specified in step b; and
c. annealing the single strand DNA overhangs and thereby creating a hairpinned inverted repeat on each end of the DNA fragment resulting from the denaturing step to produce the hairpin-ended DNA molecule.

4. A method for preparing precursors of hairpin-ended DNA molecules, wherein the method comprises:
a. providing a circular DNA molecule as a template;
b. incubating the template with a polymerase and a primer pair under conditions suitable for amplification to produce at least one amplification product and suitable for at least 2-fold amplification of the template, wherein the amplification product comprises
i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
(1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
(2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
(3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
(4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
ii. a restriction enzyme site wherein the restriction enzyme site is located outside a segment comprising the first inverted repeat at one end, the sequence of interest, and the second inverted repeat at the other end; and
c. incubating the amplification product with a restriction enzyme that cleaves the restriction enzyme site to produce the precursor of the hairpin-ended DNA molecule comprising the first inverted repeat, the sequence of interest, and the second inverted repeat.

5. The method of claim 4, wherein the template comprises no more than one type of restriction enzyme site, wherein the restriction enzyme site is present 1, 2, 3, 4, 5, 6, 7, 8, 9, 10, or more times in the template.

6. A method for preparing precursors of a hairpin-ended DNA molecules, wherein the method comprises:
a. providing a circular DNA molecule as a template;
b. incubating the template with a polymerase and a primer pair under conditions suitable for amplification to produce at least one amplification product and suitable for at least 2-fold amplification of the template, wherein the amplification product comprises
i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
(1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
(2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
(3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
(4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
ii. a fifth and a sixth restriction sites for nicking endonuclease arranged on opposite strands, wherein the fifth and sixth restriction sites are located outside a segment comprising the first inverted repeat at one end, the sequence of interest, and the second inverted repeat at the other end; and
c. incubating the amplification product with a nicking endonuclease that nicks the fifth and sixth restriction sites to produce the precursor of the hairpin-ended DNA molecule comprising the first inverted repeat, the sequence of interest, and the second inverted repeat.

7. The method of claim 6, wherein the template comprises no additional restriction sites for nicking endonuclease, optionally wherein each of the fifth and sixth restriction sites for nicking endonuclease is present 1, 2, 3, 4, 5, 6, 7, 8, 9, 10 or more times in the template.

8. The method of claim 6 or 7, wherein (i) nicks created by nicking at the fifth and sixth restriction sites are 1, 2, 3, 4, 5, 6, 7, 8, 9, 10, 11, 12, 13, 14, 15, 16, 17, 18, 19, or 20 nucleotides apart; and (ii) single strand DNA overhangs created by nicking at the fifth and sixth restriction sites do not anneal at detectable levels inter- or intramolecularly under conditions that favor annealing of the first and/or second inverted repeat.

9. The method of any one of claims 4 to 8, further comprising:
d. incubating the precursor of the hairpin-ended DNA molecule with one or more nicking endonucleases recognizing the first, second, third, and fourth restriction site;
e. denaturing and thereby creating a DNA fragment that comprises the two single strand DNA overhangs as specified in step b upon separation of the top from the bottom strand; and
f. annealing the single strand DNA overhangs of the DNA fragment and thereby creating a hairpinned inverted repeat on each end of the DNA fragment resulting from the denaturing step to produce the hairpin-ended DNA molecule.

10. The method of claim 9, wherein the method produces non-hairpin-ended DNA molecules comprising at least one non-hairpin end, and the method further comprises digesting the non-hairpin-ended DNA molecules with an exonuclease, wherein the hairpin-ended DNA molecule is resistant to digestion by the exonuclease.

11. The method of any one of claims 4-10, wherein the amplification product comprises an additional restriction enzyme site and/or additional restriction sites for nicking endonuclease located outside a segment comprising the first inverted repeat at one end, the sequence of interest, and the second inverted repeat at the other end, and the method further comprises creating additional non-hairpin-ended DNA molecules by cleaving the additional restriction enzyme site and/or nicking the additional restriction sites for nicking endonuclease.

12. A method for preparing a composition comprising pure hairpin-ended DNA molecules, wherein the method comprises:
a. providing a circular DNA molecule as a template;
b. incubating the template with a polymerase and a primer pair under conditions suitable for amplification to produce at least one amplification product and suitable for at least 2-fold amplification of the template, wherein the amplification product comprises
i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
(1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
(2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
(3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
(4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
ii. a restriction enzyme site wherein the restriction enzyme site is located outside a segment comprising the first inverted repeat at one end, the sequence of interest, and the second inverted repeat at the other end;
c. incubating the amplification product with one or more nicking endonucleases recognizing the four restriction sites;
d. denaturing and thereby creating a DNA fragment that comprises the two single strand DNA overhangs as specified in step b upon separation of the top from the bottom strand;
e. annealing the single strand DNA overhangs and thereby creating a hairpinned inverted repeat on each end of the DNA fragment resulting from the denaturing step to produce a hairpin-ended DNA molecule comprising the sequence of interest and a hairpin-ended DNA molecule comprising the restriction enzyme site;
f. incubating the hairpin-ended DNA molecule comprising the restriction enzyme site with a restriction enzyme that cleaves at the restriction enzyme site to produce a non-hairpin-ended DNA molecule comprising at least one non-hairpin end; and
g. digesting the non-hairpin-ended DNA molecules with an exonuclease, wherein the hairpin-ended DNA molecule comprising the sequence of interest is resistant to digestion by the exonuclease.

13. The method of claim 12, wherein the template comprises no more than one of the restriction enzyme site.

14. A method for preparing a composition comprising pure hairpin-ended DNA molecules, wherein the method comprises:
a. providing a circular DNA molecule as a template;
b. incubating the template with a polymerase and a primer pair under conditions suitable for amplification to produce at least one amplification product and suitable for at least 2-fold amplification of the template, wherein the amplification product comprises
i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
(1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
(2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
(3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
(4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
ii. a fifth and a sixth restriction sites for nicking endonuclease arranged on opposite strands, wherein the fifth and sixth restriction sites are located outside a segment comprising the first inverted repeat at one end, the sequence of interest, and the second inverted repeat at the other end; and
c. incubating the amplification product with one or more nicking endonucleases recognizing the four restriction sites;
d. denaturing and thereby creating a DNA fragment that comprises the two single strand DNA overhangs as specified in step b upon separation of the top from the bottom strand;
e. annealing the single strand DNA overhangs and thereby creating a hairpinned inverted repeat on each end of the DNA fragment resulting from the denaturing step to produce a hairpin-ended DNA molecule comprising the sequence of interest and a hairpin-ended DNA molecule comprising the fifth and sixth restriction site;
f. incubating the hairpin-ended DNA molecule comprising the fifth and sixth restriction sites with a nicking endonuclease that nicks the fifth and sixth restriction sites to produce a non-hairpin-ended DNA molecule comprising at least one non-hairpin end; and
g. digesting the non-hairpin-ended DNA molecules with an exonuclease, wherein the hairpin-ended DNA molecule comprising the sequence of interest is resistant to digestion by the exonuclease.

15. The method of claim 14, wherein (i) the nicks created by nicking at the fifth and sixth restriction sites are 1, 2, 3, 4, 5, 6, 7, 8, 9, 10, 11, 12, 13, 14, 15, 16, 17, 18, 19, or 20 nucleotides apart; and (ii) the single strand DNA overhangs created by nicking at the fifth and sixth restriction sites do not anneal at detectable levels inter- or intramolecularly under conditions that favor annealing of the first and/or second inverted repeat.

16. The method of any one of claims 12-15, wherein the amplification product comprises an additional restriction enzyme site and/or additional restriction sites for nicking endonuclease located outside a segment comprising the first inverted repeat at one end, the sequence of interest, and the second inverted repeat at the other end, and the method further comprises creating additional non-hairpin-ended DNA molecules by cleaving the additional restriction enzyme site and/or nicking the additional restriction sites for nicking endonuclease.

17. A method for amplifying precursors of hairpin-ended DNA molecules, wherein the method comprises:
a. providing a circular DNA molecule as a template comprising a methylated methylation-sensitive restriction enzyme (MSRE)-recognition site; and
b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
c. incubating the template with an MSRE, wherein the amplification product comprises an unmethylated MSRE-recognition site located outside a segment comprising the first inverted repeat at one end, the sequence of interest, and the second inverted repeat at the other end, and the MSRE cleaves the amplification product at the unmethylated MSRE-recognition site.

18. The method of claim 17, wherein the circular DNA molecule is incubated with the polymerase and the MSRE concurrently, or the circular DNA molecule is incubated with the polymerase prior to the MSRE.

19. The method of claim 17 or 18, further comprising:
d. incubating the MSRE-cleaved amplification products with one or more nicking endonucleases recognizing the four restriction sites, thereby creating the two single strand DNA overhangs as specified in step b upon separation of the top from the bottom strand;
e. denaturing and thereby creating a DNA fragment that comprises the two single strand DNA overhangs; and
f. annealing the single strand DNA overhangs and thereby creating a hairpinned inverted repeat on each end of the DNA fragment resulting from the denaturing step to produce a hairpin-ended DNA molecule.

20. A method for preparing hairpin-ended DNA molecules, wherein the method comprises:
a. providing a circular DNA molecule comprising a methylated methylation-sensitive nicking endonuclease (MSNE)-restriction site;
b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
c. incubating the template with an MSNE, wherein the amplification product comprises two unmethylated MSNE-recognition sites located outside a segment comprising the first inverted repeat at one end, the sequence of interest, and the second inverted repeat at the other end, and the MSNE cleaves the amplification product at the two unmethylated MSNE-recognition sites.

21. The method of claim 20, wherein the circular DNA molecule is incubated with the polymerase and the MSNE concurrently, or the circular DNA molecule is incubated with the polymerase prior to the MSNE.

22. The method of claim 20 or 21, further comprising:
d. incubating the MSNE-cleaved amplification products with one or more nicking endonucleases recognizing the four restriction sites, thereby creating the two single strand DNA overhangs as specified in step b upon separation of the top from the bottom strand;
e. denaturing and thereby creating a DNA fragment that comprises the two single strand DNA overhangs; and
f. annealing the single strand DNA overhangs and thereby creating a hairpinned inverted repeat on each end of the DNA fragment resulting from the denaturing step to produce a hairpin-ended DNA molecule.

23. A method for preparing hairpin-ended DNA molecules, wherein the method comprises:
a. providing a circular DNA molecule as a template comprising a methylated methylation-sensitive restriction enzyme (MSRE)-recognition site; and
b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand;
c. incubating the template with an MSRE, wherein the amplification product comprises an unmethylated MSRE-recognition site located outside the segment comprising the first inverted repeat at one end, the sequence of interest, and the second inverted repeat at the other end, and the MSRE cleaves the amplification product at the unmethylated MSRE-recognition site;
d. incubating the MSRE-cleaved amplification products with one or more nicking endonucleases recognizing the four restriction sites, thereby creating the two single strand DNA overhangs as specified in step b upon separation of the top from the bottom strand;
e. denaturing and thereby creating a DNA fragment that comprises the two single strand DNA overhangs; and
f. annealing the single strand DNA overhangs and thereby creating a hairpinned inverted repeat on each end of the DNA fragment resulting from the denaturing step to produce a hairpin-ended DNA molecule.

24. The method of claim 23, wherein the method produces non-hairpin-ended DNA molecules comprising at least one non-hairpin end, and the method further comprises digesting the non-hairpin-ended DNA molecules with an exonuclease, wherein the hairpin-ended DNA molecule is resistant to digestion by the exonuclease.

25. A method for preparing hairpin-ended DNA molecules, wherein the method comprises:
a. providing a circular DNA molecule comprising a methylated methylation-sensitive nicking endonuclease (MSNE)-restriction site;
b. incubating the template with a polymerase and a primer pair under conditions suitable for amplification to produce at least one amplification product and suitable for at least 2-fold amplification of the template, wherein the amplification product comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
c. incubating the template with an MSNE, wherein the amplification product comprises two unmethylated MSNE-recognition sites located outside the segment comprising the first inverted repeat at one end, the sequence of interest, and the second inverted repeat at the other end, and the MSNE cleaves the amplification product at the two unmethylated MSNE-recognition sites;
d. incubating the MSNE-cleaved amplification products with one or more nicking endonucleases recognizing the four restriction sites, thereby creating the two single strand DNA overhangs as specified in step b upon separation of the top from the bottom strand;
e. denaturing and thereby creating a DNA fragment that comprises the two single strand DNA overhangs; and
f. annealing the single strand DNA overhangs and thereby creating a hairpinned inverted repeat on each end of the DNA fragment resulting from the denaturing step to produce a hairpin-ended DNA molecule.

26. The method of claim 25, wherein (i) the nicks created by nicking at the two unmethylated restriction sites are 1, 2, 3, 4, 5, 6, 7, 8, 9, 10, 11, 12, 13, 14, 15, 16, 17, 18, 19, or 20 nucleotides apart; and (ii) the single strand DNA overhangs created by nicking at the two unmethylated restriction sites do not anneal at detectable levels inter- or intramolecularly under conditions that favor annealing of the first and/or second inverted repeat.

27. The method of claim 25 or 26, wherein the method further produces non-hairpin-ended DNA molecules comprising at least one non-hairpin end, and the method further comprises digesting the non-hairpin-ended DNA molecules with one or more exonucleases, wherein the hairpin-ended DNA molecule is resistant to digestion by the one or more exonucleases.

28. The method of any one of claims 1 to 16, further comprising exchanging a buffer, concentrating the amplification product, and/or removing the circular DNA molecule, polymerase, and/or primer pair, after completion of step b and before initiation of step c.

29. The method of any one of claims 17 to 27, further comprising exchanging a buffer, concentrating the amplification product, and/or removing the circular DNA molecule, polymerase, and/or primer pair, after completion of step c and before initiation of step d.

30. The method of claim any one of claims 1 to 29, wherein the sequence of interest comprises a transcription unit encoding a therapeutic protein.

31. The method of any one of claims 1 to 29, wherein the sequence of interest comprises a transcription unit encoding an RNA for in vitro transcription (IVT).

32. The method of any one of claims 1 to 29, wherein the sequence of interest comprises a gene promoter, an AAV ITR, or a synthetic DNA template to be integrated into a genome.

33. The method of any one of claims 1-32, wherein the circular DNA molecule is a single-stranded circular DNA molecule or a double stranded circular DNA molecule.

34. The method of any one of claims 1-33, wherein the amplification is an isothermal amplification.

35. The method of claim 34, wherein the isothermal amplification is rolling circle amplification (RCA) and/or multiple displacement amplification (MDA).

36. A kit for preparing hairpin-ended DNA molecules, comprising:
a. a circular DNA molecule as a template, wherein an amplification product amplified from the template comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand;
b. a DNA polymerase suitable for amplification;
c. a primer pair; and
d. one or more nicking endonucleases recognizing the four restriction sites in the amplification product.

37. A kit for amplifying precursors of hairpin-ended DNA molecules, comprising:
a. a circular DNA molecule as a template, wherein an amplification product amplified from the template comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand;
b. a DNA polymerase suitable for amplification; and
c. a primer pair.

38. The kit of claim 37, further comprising one or more nicking endonucleases recognizing the four restriction sites in the amplification product.

39. A kit for preparing precursors of hairpin-ended DNA molecules, comprising:
a. a circular DNA molecule as a template, wherein an amplification product amplified from the template comprises:
i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
(1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
(2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
(3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
(4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
ii. a restriction enzyme site wherein the restriction enzyme site is located outside a segment comprising the first inverted repeat at one end, the sequence of interest, and the second inverted repeat at the other end;
b. a DNA polymerase suitable for amplification;
c. a primer pair; and
d. a restriction enzyme that recognizes the restriction enzyme site.

40. A kit for preparing precursors of hairpin-ended DNA molecules, comprising:
a. a circular DNA molecule as a template, wherein an amplification product amplified from the template comprises:
i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
(1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
(2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
(3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
(4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
ii. a fifth and a sixth restriction sites for nicking endonuclease arranged on opposite strands, wherein the fifth and sixth restriction sites are located outside a segment comprising the first inverted repeat at one end, the sequence of interest, and the second inverted repeat at the other end;
b. a DNA polymerase suitable for amplification;
c. a primer pair; and
d. a nicking endonuclease that recognizes the fifth and a sixth restriction site.

41. The kit of claim 39 or 40, further comprising one or more nicking endonucleases recognizing the first, second, third, and forth restriction sites in the amplification product.

42. A kit for preparing a composition comprising pure hairpin-ended DNA molecules, comprising:
a. a circular DNA molecule as a template, wherein an amplification product amplified from the template comprises:
i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
(1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
(2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
(3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
(4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
ii. a restriction enzyme site wherein the restriction enzyme site is located outside a segment comprising the first inverted repeat at one end, the sequence of interest, and the second inverted repeat at the other end;
b. a DNA polymerase suitable for amplification;
c. a primer pair;
d. a restriction enzyme that recognizes the restriction enzyme site;
e. one or more nicking endonucleases that recognizes the first, second, third, and forth restriction sites in the amplification product; and
f. an exonuclease.

43. A kit for preparing a composition comprising pure hairpin-ended DNA molecules, comprising:
a. a circular DNA molecule as a template, wherein an amplification product amplified from the template comprises:
i. a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
(1) the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
(2) the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
(3) the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
(4) the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
ii. a fifth and a sixth restriction sites for nicking endonuclease arranged on opposite strands, wherein the fifth and sixth restriction sites are located outside a segment comprising the first inverted repeat at one end, the sequence of interest, and the second inverted repeat at the other end;
b. a DNA polymerase suitable for amplification;
c. a primer pair;
d. a nicking endonuclease that recognizes the fifth and a sixth restriction site;
e. one or more nicking endonucleases that recognizes the first, second, third, and forth restriction sites in the amplification product; and
f. an exonuclease.

44. A kit for amplifying precursors of hairpin-ended DNA molecules, comprising:
a. a circular DNA molecule as a template comprising a methylated methylation-sensitive restriction enzyme (MSRE)-recognition site, wherein an amplification product amplified from the template comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
b. an MSRE that recognizes and cleaves the amplification product at an unmethylated MSRE-recognition site located outside a segment comprising the first inverted repeat at one end, the sequence of interest, and the second inverted repeat at the other end;
c. a DNA polymerase suitable for amplification; and
d. a primer pair.

45. A kit for preparing hairpin-ended DNA molecules, comprising:
a. a circular DNA molecule comprising a methylated methylation-sensitive nicking endonuclease (MSNE)-restriction site, wherein an amplification product amplified from the template comprises a top strand and a bottom strand and, in 5′ to 3′ direction of the top strand, a first inverted repeat, a sequence of interest, and a second inverted repeat, wherein a first and a second restriction sites for nicking endonuclease are arranged on opposite strands in proximity of the first inverted repeat and a third and a fourth restriction sites for nicking endonuclease are arranged on opposite strands in proximity of the second inverted repeat, and wherein:
i. the first, second, third, and fourth restriction sites are arranged such that nicking results in a top strand 5′ overhang comprising the first inverted repeat or a fragment thereof and a top strand 3′ overhang comprising the second inverted repeat or a fragment thereof upon separation of the top from the bottom strand;
ii. the first, second, third, and fourth restriction sites are arranged such that nicking results in a bottom strand 3′ overhang comprising the first inverted repeat or a fragment thereof and a bottom strand 5′ overhang comprising the second inverted repeat or a fragment thereof upon separation of the top from the bottom strand;
iii. the first, second, third, and fourth restriction sites are arranged such that nicking results in a top strand 5′ overhang comprising the first inverted repeat or a fragment thereof and a bottom strand 5′ overhang comprising the second inverted repeat or a fragment thereof upon separation of the top from the bottom strand; or
iv. the first, second, third, and fourth restriction sites are arranged such that nicking results in a bottom strand 3′ overhang comprising the first inverted repeat or a fragment thereof and a top strand 3′ overhang comprising the second inverted repeat or a fragment thereof upon separation of the top from the bottom strand; and
b. an MSNE that recognizes and nicks the amplification product at the two unmethylated MSNE-recognition sites located outside a segment comprising the first inverted repeat at one end, the sequence of interest, and the second inverted repeat at the other end;
c. a DNA polymerase suitable for amplification; and
d. a primer pair.

46. The kit of claim 44 or 45, further comprising one or more nicking endonucleases that recognize the first, second, third, and forth restriction sites in the amplification product.

47. The kit of claim 46, further comprising an exonuclease.

48. The kit of any one of claims 39-47, wherein the amplification is an isothermal amplification.

49. The kit of claim 48, wherein the isothermal amplification is rolling circle amplification (RCA) and/or multiple displacement amplification (MDA).

50. A method of producing AAV vectors for use in gene therapy comprising:
a. transfecting a host cell with at least one hairpin-ended DNA molecule for production of AAV particles, wherein the hairpin-ended DNA molecule has been produced according to the method of any one of claims 1 to 36 and/or using the kit of any one of claims 36 to 49; and
b. harvesting the AAV particles.

51. The method of claim 50, wherein step a comprises co-transfecting the host cell with (i) a hairpin-ended DNA molecule encoding an AAV vector genome and (ii) one or more DNA molecules encoding Rep protein(s), AAV capsid protein(s), and/or helper plasmid(s).

52. The method of claim 50, wherein step a comprises co-transfecting the host cell with (i) a hairpin-ended DNA molecule encoding an AAV vector genome; (ii) a hairpin-ended DNA molecule encoding Rep proteins and AAV capsid proteins; and (iii) a hairpin-ended DNA molecule encoding helper plasmids.

53. A method of producing lentiviral vectors for use in gene therapy comprising:
a. transfecting a host cell with at least one hairpin-ended DNA molecule for production of lentiviral particles, wherein the hairpin-ended DNA molecule has been produced according to the method of any one of claims 1 to 36 and/or using the kit of any one of claims 36 to 49; and
b. harvesting the lentiviral particles.

54. The method of claim 53, wherein step a comprises co-transfecting the host cell with (i) a hairpin-ended DNA molecule encoding a lentiviral transfer vector and (ii) one or more DNA molecules encoding packaging and/or envelope proteins selected from the group consisting of VSV-G protein(s)), Tat proteins, Rev protein(s), Gag protein(s), and Pol protein(s).

55. The method of claim 53, wherein step a comprises co-transfecting the host cell with (i) a hairpin-ended DNA molecule encoding a lentiviral transfer vector; (ii) a hairpin-ended DNA molecule encoding Rev protein; (iii) a hairpin-ended DNA molecule encoding Gag and Pol proteins; and (iv) a hairpin-ended DNA molecule encoding VSV-G protein.

56. A method of producing RNA comprising:
a. transcribing a hairpin-ended DNA molecule, or a fragment thereof, for production of RNA, wherein the hairpin-ended DNA molecule comprises a transcription unit suitable for in vitro transcription (IVT) and has been produced according to the method of any one of claims 1 to 36;
b. harvesting the RNA product.

57. The method of claim 56, wherein the transcribing comprises the contacting the hairpin-ended DNA molecule, or fragment thereof, with an in vitro transcription reaction system comprising an RNA polymerase and ribonucleotides.


SEQUENCE LISTING (535 sequences)

SEQ ID NO: 1 - DNA, 85 bp, Synthetic construct
  source 1..85: mol_type = other DNA
gctcgactcgctcactgaggccgggcgaccaaaggtcgcccgacgcccgggctttgcccgggcggcctcagtgagcgagtcgagc

SEQ ID NO: 2 - DNA, 77 bp, Synthetic construct
  source 1..77: mol_type = other DNA
gctcgactcactgaggccgggcgaccaaaggtcgcccgacgcccgggctttgcccgggcggcctcagtgagtcgagc

SEQ ID NO: 3 - DNA, 69 bp, Synthetic construct
  source 1..69: mol_type = other DNA
cgctgactcaggccgggcgaccaaaggtcgcccgacgcccgggctttgcccgggcggcctgagtcagcg

SEQ ID NO: 4 - DNA, 89 bp, Synthetic construct
  source 1..89: mol_type = other DNA
cgcgctcgctcgctcactgaggccgggcgaccaaaggtcgcccgacgcccgggctttgcccgggcggcctcagtgagcgagcgagcgcg

SEQ ID NO: 5 - DNA, 71 bp, Synthetic construct
  source 1..71: mol_type = other DNA
tcgctcactgaggccgggcgaccaaaggtcgcccgacgcccgggctttgcccgggcggcctcagtgagcga

SEQ ID NO: 6 - DNA, 59 bp, Synthetic construct
  source 1..59: mol_type = other DNA
actgaggccgggcgaccaaaggtcgcccgacgcccgggctttgcccgggcggcctcagt

SEQ ID NO: 7 - DNA, 51 bp, Synthetic construct
  source 1..51: mol_type = other DNA
aggccgggcgaccaaaggtcgcccgacgcccgggctttgcccgggcggcct

SEQ ID NO: 8 - DNA, 70 bp, Synthetic construct
  source 1..70: mol_type = other DNA
ccatgcatccggctttaaacgggcaactgcgtctcattcacgttagagactacaaccgtcggatgcatgg

SEQ ID NO: 9 - DNA, 102 bp, Synthetic construct
  source 1..102: mol_type = other DNA
ttcaaacctgccgggggagaagcggcgttttttcccggccgccgcttctcttcttctcccgccgccgggaaaaaaggcgggagaagccccggcaggtttgaa

SEQ ID NO: 10 - DNA, 92 bp, Synthetic construct
  source 1..92: mol_type = other DNA
gtccgggccatgcttcaaacctgccggggcttctcccgccttttttcccggcggcgggagaagtagatttctcgtacctgcatggcccggac

SEQ ID NO: 11 - DNA, 87 bp, Synthetic construct
  source 1..87: mol_type = other DNA
ccagcgcttggggttgacgtgccactaagatcaagcggcgcgcgcgcgccgcttgtcttagtgtcaaggcaaccccaagcaagctgg

SEQ ID NO: 12 - DNA, 113 bp, Synthetic construct
  source 1..113: mol_type = other DNA
ggttgactctgggccagcttgcttggggttgccttgacactaagacaagcggcgcgcgcgcgccgcttgatcttagtggcacgtcaaccccaagcgctggcccagagtcaacc

SEQ ID NO: 13 - DNA, 83 bp, Synthetic construct
  source 1..83: mol_type = other DNA
cgcgctcgctcgctcactgaggccgggccaaaggcccgacgcccgggctttgcccgggcggcctcagtgagcgagcgagcgcg

SEQ ID NO: 14 - DNA, 83 bp, Synthetic construct
  source 1..83: mol_type = other DNA
cgcgctcgctcgctcactgaggccgggcgaccaaaggtcgcccgacgcccgtttcgggcggcctcagtgagcgagcgagcgcg

SEQ ID NO: 15 - DNA, 67 bp, Synthetic construct
  source 1..67: mol_type = other DNA
cgcgctcgctcgctcactgaggccgcccgggctttgcccgggcggcctcagtgagcgagcgagcgcg

SEQ ID NO: 16 - DNA, 125 bp, Adeno-associated virus
  source 1..125: mol_type = genomic DNA; note = AAV1 Left ITR
ttgcccactccctctctgcgcgctcgctcgctcggtggggcctgcggaccaaaggtccgcagacggcagagctctgctctgccggccccaccgagcgagcgagcgcgcagagagggagtgggcaa

SEQ ID NO: 17 - DNA, 125 bp, Adeno-associated virus; identical to SEQ ID NO: 16
  source 1..125: mol_type = genomic DNA; note = AAV1 Right ITR

SEQ ID NO: 18 - DNA, 125 bp, Adeno-associated virus
  source 1..125: mol_type = genomic DNA; note = AAV2 Left ITR
ttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 19 - DNA, 125 bp, Adeno-associated virus; identical to SEQ ID NO: 18
  source 1..125: mol_type = genomic DNA; note = AAV2 Right ITR

SEQ ID NO: 20 - DNA, 125 bp, Adeno-associated virus
  source 1..125: mol_type = genomic DNA; note = AAV3 Left ITR
ttggccactccctctatgcgcactcgctcgctcggtggggcctggcgaccaaaggtcgccagacggacgtgctttgcacgtccggccccaccgagcgagcgagtgcgcatagagggagtggccaa

SEQ ID NO: 21 - DNA, 125 bp, Adeno-associated virus; identical to SEQ ID NO: 20
  source 1..125: mol_type = genomic DNA; note = AAV3 Right ITR

SEQ ID NO: 22 - DNA, 125 bp, Adeno-associated virus
  source 1..125: mol_type = genomic DNA; note = AAV4 Left ITR
ttggccactccctctatgcgcgctcgctcactcactcggccctggagaccaaaggtctccagactgccggcctctggccggcagggccgagtgagtgagcgagcgcgcatagagggagtggccaa

SEQ ID NO: 23 - DNA, 112 bp, Adeno-associated virus
  source 1..112: mol_type = genomic DNA; note = AAV4 Right ITR
ctatgcgcgctcgctcactcactcggccctggagaccaaaggtctccagactgccggcctctggccggcagggccgagtgagtgagcgagcgcgcatagagggagtggccaa

SEQ ID NO: 24 - DNA, 137 bp, Adeno-associated virus
  source 1..137: mol_type = genomic DNA; note = AAV5 (NC_006152) Left ITR
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 25 - DNA, 137 bp, Adeno-associated virus; identical to SEQ ID NO: 24
  source 1..137: mol_type = genomic DNA; note = AAV5 (NC_006152) Right ITR

SEQ ID NO: 26 - DNA, 125 bp, Adeno-associated virus
  source 1..125: mol_type = genomic DNA; note = AAV7 (NC_006260) Left ITR
ttggccactccctctatgcgcgctcgctcgctcggtggggcctgcggaccaaaggtccgcagacggcagagctctgctctgccggccccaccgagcgagcgagcgcgcatagagggagtggccaa

SEQ ID NO: 27 - DNA, 125 bp, Adeno-associated virus; identical to SEQ ID NO: 26
  source 1..125: mol_type = genomic DNA; note = AAV7 (NC_006260) Right ITR

SEQ ID NO: 28 - DNA, 140 bp, Adeno-associated virus
  source 1..140: mol_type = genomic DNA; note = HBOV (JQ923422) Left ITR
gtggttgtacagacgccatcttggaatccaatatgtctgccggctcagtcatgcctgcgctgcgcgcagcgcgctgcgcgcgcgcatgatctaatcgccggcagacatattggattccaagatggcgtctgtacaaccac

SEQ ID NO: 29 - DNA, 200 bp, Adeno-associated virus
  source 1..200: mol_type = genomic DNA; note = HBOV (JQ923422) Right ITR
ttgcttatgcaatcgcgaaactctatatcttttaatgtgttgttgttgtacatgcgccatcttagttttatatcagctggcgccttagttatataacatgcatgttatataactaaggcgccagctgatataaaactaagatggcgcatgtacaacaacaacacattaaaagatatagagtttcgcgattgcataagcaa

SEQ ID NO: 30 - DNA, 89 bp, Adeno-associated virus
  source 1..89: mol_type = genomic DNA; note = hB19 (AY386330) Left ITR
tgggccagcttgcttggggttgccttgacactaagacaagcggcgcgccgcttgatcttagtggcacgtcaaccccaagcgctggccca

SEQ ID NO: 31 - DNA, 89 bp, Adeno-associated virus
  source 1..89: mol_type = genomic DNA; note = hB19 (AY386330) Right ITR
tgggccagcgcttggggttgacgtgccactaagatcaagcggcgcgccgcttgtcttagtgtcaaggcaaccccaagcaagctggccca

SEQ ID NO: 32 - DNA, 50 bp, Synthetic construct
  source 1..50: mol_type = other DNA; note = OriL hairpin sequence
cttctcccgccgccgggaaaaaaggcgggagaagccccggcaggtttgaa

SEQ ID NO: 33 - skipped

SEQ ID NO: 34 - DNA, 125 bp, Synthetic construct
  source 1..125: mol_type = other DNA; note = source: AAV1; Recogn. Site: Nb.BbvCI; Format: bl
ttgcccactccccctcagcgcgctcgctcgctcggtggggcctgcggaccaaaggtccgcagacggcagagctctgctctgccggccccaccgagcgagcgagcgcgctgagggggagtgggcaa

SEQ ID NO: 35 - DNA, 125 bp, Synthetic construct
  source 1..125: mol_type = other DNA; note = source: AAV1; Recogn. Site: Nb.BbvCI; Format: tl
ttgcccactcccgctgagggcgctcgctcgctcggtggggcctgcggaccaaaggtccgcagacggcagagctctgctctgccggccccaccgagcgagcgagcgccctcagcgggagtgggcaa

SEQ ID NO: 36 - DNA, 125 bp, Synthetic construct
  source 1..125: mol_type = other DNA; note = source: AAV2; Recogn. Site: Nb.BbvCI; Format: bl
ttggccactccccctcagcgcgctcgctcgctcactgaggccgggcgaccaaaggtcgcccgacgcccgggctttgcccgggcggcctcagtgagcgagcgagcgcgctgagggggagtggccaa

SEQ ID NO: 37 - DNA, 125 bp, Synthetic construct
  source 1..125: mol_type = other DNA; note = source: AAV2; Recogn. Site: Nb.BbvCI; Format: tl
ttggccactcccgctgagggcgctcgctcgctcactgaggccgggcgaccaaaggtcgcccgacgcccgggctttgcccgggcggcctcagtgagcgagcgagcgccctcagcgggagtggccaa

SEQ ID NO: 38 - DNA, 125 bp, Synthetic construct
  source 1..125: mol_type = other DNA; note = source: AAV3; Recogn. Site: Nb.BbvCI; Format: bl
ttggccactccccctcagcgcactcgctcgctcggtggggcctggcgaccaaaggtcgccagacggacgtgctttgcacgtccggccccaccgagcgagcgagtgcgctgagggggagtggccaa

SEQ ID NO: 39 - DNA, 125 bp, Synthetic construct
  source 1..125: mol_type = other DNA; note = source: AAV3; Recogn. Site: Nb.BbvCI; Format: tl
ttggccactcccgctgagggcactcgctcgctcggtggggcctggcgaccaaaggtcgccagacggacgtgctttgcacgtccggccccaccgagcgagcgagtgccctcagcgggagtggccaa

SEQ ID NO: 40 - DNA, 125 bp, Synthetic construct
  source 1..125: mol_type = other DNA; note = source: AAV4 left; Recogn. Site: Nb.BbvCI; Format: bl
ttggccactccccctcagcgcgctcgctcactcactcggccctggagaccaaaggtctccagactgccggcctctggccggcagggccgagtgagtgagcgagcgcgctgagggggagtggccaa

SEQ ID NO: 41 - DNA, 125 bp, Synthetic construct
  source 1..125: mol_type = other DNA; note = source: AAV4 left; Recogn. Site: Nb.BbvCI; Format: tl
ttggccactcccgctgagggcgctcgctcactcactcggccctggagaccaaaggtctccagactgccggcctctggccggcagggccgagtgagtgagcgagcgccctcagcgggagtggccaa

SEQ ID NO: 42 - DNA, 125 bp, Synthetic construct
  source 1..125: mol_type = other DNA; note = source: AAV4 right; Recogn. Site: Nb.BbvCI; Format: bl
ttggccacattacctcagcgcgctcgctcactcactcggccctggagaccaaaggtctccagactgccggcctctggccggcagggccgagtgagtgagcgagcgcgctgagggggagtggccaa

SEQ ID NO: 43 - DNA, 125 bp, Synthetic construct
  source 1..125: mol_type = other DNA; note = source: AAV4 right; Recogn. Site: Nb.BbvCI; Format: tl
ttggccacattagctgagggcgctcgctcactcactcggccctggagaccaaaggtctccagactgccggcctctggccggcagggccgagtgagtgagcgagcgccctcagcgggagtggccaa

SEQ ID NO: 44 - DNA, 137 bp, Synthetic construct
  source 1..137: mol_type = other DNA; note = source: AAV5; Recogn. Site: Nb.BbvCI; Format: bl
ctctcccctcagccgcgttcgctcgctcgctggctcgtttgggggggtggcagctcaaagagctgccagacgacggccctctggccgtcgcccccccaaacgagccagcgagcgagcgaacgcggctgaggggagag

SEQ ID NO: 45 - DNA, 137 bp, Synthetic construct
  source 1..137: mol_type = other DNA; note = source: AAV5; Recogn. Site: Nb.BbvCI; Format: tl
ctctccccgctgaggcgttcgctcgctcgctggctcgtttgggggggtggcagctcaaagagctgccagacgacggccctctggccgtcgcccccccaaacgagccagcgagcgagcgaacgcctcagcggggagag

SEQ ID NO: 46 - DNA, 125 bp, Synthetic construct
  source 1..125: mol_type = other DNA; note = source: AAV7; Recogn. Site: Nb.BbvCI; Format: bl
ttggccactccccctcagcgcgctcgctcgctcggtggggcctgcggaccaaaggtccgcagacggcagagctctgctctgccggccccaccgagcgagcgagcgcgctgagggggagtggccaa

SEQ ID NO: 47 - DNA, 125 bp, Synthetic construct
  source 1..125: mol_type = other DNA; note = source: AAV7; Recogn. Site: Nb.BbvCI; Format: tl
ttggccactcccgctgagggcgctcgctcgctcggtggggcctgcggaccaaaggtccgcagacggcagagctctgctctgccggccccaccgagcgagcgagcgccctcagcgggagtggccaa

SEQ ID NO: 48 - DNA, 125 bp, Synthetic construct
  source 1..125: mol_type = other DNA; note = source: AAV1; Recogn. Site: Nb.BsmI; Format: bl
ttgcccactccctgaatgcgcgctcgctcgctcggtggggcctgcggaccaaaggtccgcagacggcagagctctgctctgccggccccaccgagcgagcgagcgcgcattcagggagtgggcaa

SEQ ID NO: 49 - DNA, 125 bp, Synthetic construct
  source 1..125: mol_type = other DNA; note = source: AAV1; Recogn. Site: Nb.BsmI; Format: tl
ttgcccactccctctctgcgcattcgctcgctcggtggggcctgcggaccaaaggtccgcagacggcagagctctgctctgccggccccaccgagcgagcgaatgcgcagagagggagtgggcaa

SEQ ID NO: 50 - DNA, 125 bp, Synthetic construct
  source 1..125: mol_type = other DNA; note = source: AAV2; Recogn. Site: Nb.BsmI; Format: bl
ttggccactccctgaatgcgcgctcgctcgctcactgaggccgggcgaccaaaggtcgcccgacgcccgggctttgcccgggcggcctcagtgagcgagcgagcgcgcattcagggagtggccaa

SEQ ID NO: 51 - DNA, 125 bp, Synthetic construct
  source 1..125: mol_type = other DNA; note = source: AAV2; Recogn. Site: Nb.BsmI; Format: tl
ttggccactccctctctgcgcattcgctcgctcactgaggccgggcgaccaaaggtcgcccgacgcccgggctttgcccgggcggcctcagtgagcgagcgaatgcgcagagagggagtggccaa

SEQ ID NO: 52 - DNA, 125 bp, Synthetic construct
  source 1..125: mol_type = other DNA; note = source: AAV3; Recogn. Site: Nb.BsmI; Format: bl
ttggccactccctgaatgcgcactcgctcgctcggtggggcctggcgaccaaaggtcgccagacggacgtgctttgcacgtccggccccaccgagcgagcgagtgcgcattcagggagtggccaa

SEQ ID NO: 53 - DNA, 125 bp, Synthetic construct
  source 1..125: mol_type = other DNA; note = source: AAV3; Recogn. Site: Nb.BsmI; Format: tl
ttggccactccctctatgcgcattcgctcgctcggtggggcctggcgaccaaaggtcgccagacggacgtgctttgcacgtccggccccaccgagcgagcgaatgcgcatagagggagtggccaa

SEQ ID NO: 54 - DNA, 125 bp, Synthetic construct
  source 1..125: mol_type = other DNA; note = source: AAV4 left; Recogn. Site: Nb.BsmI; Format: bl
ttggccactccctgaatgcgcgctcgctcactcactcggccctggagaccaaaggtctccagactgccggcctctggccggcagggccgagtgagtgagcgagcgcgcattcagggagtggccaa

SEQ ID NO: 55 - DNA, 125 bp, Synthetic construct
  source 1..125: mol_type = other DNA; note = source: AAV4 left; Recogn. Site: Nb.BsmI; Format: tl
ttggccactccctctatgcgcattcgctcactcactcggccctggagaccaaaggtctccagactgccggcctctggccggcagggccgagtgagtgagcgaatgcgcatagagggagtggccaa

SEQ ID NO: 56 - DNA, 125 bp, Synthetic construct
  source 1..125: mol_type = other DNA; note = source: AAV4 right; Recogn. Site: Nb.BsmI; Format: bl
ttggccacattaggaatgcgcgctcgctcactcactcggccctggagaccaaaggtctccagactgccggcctctggccggcagggccgagtgagtgagcgagcgcgcattcagggagtggccaa

SEQ ID NO: 57 - DNA, 125 bp, Synthetic construct
  source 1..125: mol_type = other DNA; note = source: AAV4 right; Recogn. Site: Nb.BsmI; Format: tl
ttggccacattagctatgcgcattcgctcactcactcggccctggagaccaaaggtctccagactgccggcctctggccggcagggccgagtgagtgagcgaatgcgcatagagggagtggccaa

SEQ ID NO: 58 - DNA, 137 bp, Synthetic construct
  source 1..137: mol_type = other DNA; note = source: AAV5; Recogn. Site: Nb.BsmI; Format: bl
ctctccccgaatgcgcgttcgctcgctcgctggctcgtttgggggggtggcagctcaaagagctgccagacgacggccctctggccgtcgcccccccaaacgagccagcgagcgagcgaacgcgcattcggggagag

SEQ ID NO: 59 - DNA, 137 bp, Synthetic construct
  source 1..137: mol_type = other DNA; note = source: AAV5; Recogn. Site: Nb.BsmI; Format: tl
ctctcccccctgtcgcattcgctcgctcgctggctcgtttgggggggtggcagctcaaagagctgccagacgacggccctctggccgtcgcccccccaaacgagccagcgagcgagcgaatgcgacaggggggagag

SEQ ID NO: 60 - DNA, 125 bp, Synthetic construct
  source 1..125: mol_type = other DNA; note = source: AAV7; Recogn. Site: Nb.BsmI; Format: bl
ttggccactccctgaatgcgcgctcgctcgctcggtggggcctgcggaccaaaggtccgcagacggcagagctctgctctgccggccccaccgagcgagcgagcgcgcattcagggagtggccaa

SEQ ID NO: 61 - DNA, 125 bp, Synthetic construct
  source 1..125: mol_type = other DNA; note = source: AAV7; Recogn. Site: Nb.BsmI; Format: tl
ttggccactccctctatgcgcattcgctcgctcggtggggcctgcggaccaaaggtccgcagacggcagagctctgctctgccggccccaccgagcgagcgaatgcgcatagagggagtggccaa

SEQ ID NO: 62 - DNA, 125 bp, Synthetic construct
  source 1..125: mol_type = other DNA; note = source: AAV1; Recogn. Site: Nb.BsrDI; Format: bl
ttgcccactcccgcaatgcgcgctcgctcgctcggtggggcctgcggaccaaaggtccgcagacggcagagctctgctctgccggccccaccgagcgagcgagcgcgcattgcgggagtgggcaa

SEQ ID NO: 63 - DNA, 125 bp, Synthetic construct
  source 1..125: mol_type = other DNA; note = source: AAV1; Recogn. Site: Nb.BsrDI; Format: tl
ttgcccactccctcattgcgcgctcgctcgctcggtggggcctgcggaccaaaggtccgcagacggcagagctctgctctgccggccccaccgagcgagcgagcgcgcaatgagggagtgggcaa

SEQ ID NO: 64 - DNA, 125 bp, Synthetic construct
  source 1..125: mol_type = other DNA; note = source: AAV2; Recogn. Site: Nb.BsrDI; Format: bl
ttggccactcccgcaatgcgcgctcgctcgctcactgaggccgggcgaccaaaggtcgcccgacgcccgggctttgcccgggcggcctcagtgagcgagcgagcgcgcattgcgggagtggccaa

SEQ ID NO: 65 - DNA, 125 bp, Synthetic construct
  source 1..125: mol_type = other DNA; note = source: AAV2; Recogn. Site: Nb.BsrDI; Format: tl
ttggccactccctcattgcgcgctcgctcgctcactgaggccgggcgaccaaaggtcgcccgacgcccgggctttgcccgggcggcctcagtgagcgagcgagcgcgcaatgagggagtggccaa

SEQ ID NO: 66 - DNA, 125 bp, Synthetic construct
  source 1..125: mol_type = other DNA; note = source: AAV3; Recogn. Site: Nb.BsrDI; Format: bl
ttggccactcccgcaatgcgcactcgctcgctcggtggggcctggcgaccaaaggtcgccagacggacgtgctttgcacgtccggccccaccgagcgagcgagtgcgcattgcgggagtggccaa

SEQ ID NO: 67 - DNA, 125 bp, Synthetic construct
  source 1..125: mol_type = other DNA; note = source: AAV3; Recogn. Site: Nb.BsrDI; Format: tl
ttggccactccctcattgcgcactcgctcgctcggtggggcctggcgaccaaaggtcgccagacggacgtgctttgcacgtccggccccaccgagcgagcgagtgcgcaatgagggagtggccaa

SEQ ID NO: 68 - DNA, 125 bp, Synthetic construct
  source 1..125: mol_type = other DNA; note = source: AAV4 left; Recogn. Site: Nb.BsrDI; Format: bl
ttggccactcccgcaatgcgcgctcgctcactcactcggccctggagaccaaaggtctccagactgccggcctctggccggcagggccgagtgagtgagcgagcgcgcattgcgggagtggccaa

SEQ ID NO: 69 - DNA, 125 bp, Synthetic construct
  source 1..125: mol_type = other DNA; note = source: AAV4 left; Recogn. Site: Nb.BsrDI; Format: tl
ttggccactccctcattgcgcgctcgctcactcactcggccctggagaccaaaggtctccagactgccggcctctggccggcagggccgagtgagtgagcgagcgcgcaatgagggagtggccaa

SEQ ID NO: 70 - DNA, 125 bp, Synthetic construct
  source 1..125: mol_type = other DNA; note = source: AAV4 right; Recogn. Site: Nb.BsrDI; Format: bl
ttggccacattagcaatgcgcgctcgctcactcactcggccctggagaccaaaggtctccagactgccggcctctggccggcagggccgagtgagtgagcgagcgcgcattgcgggagtggccaa

SEQ ID NO: 71 - DNA, 125 bp, Synthetic construct
  source 1..125: mol_type = other DNA; note = source: AAV4 right; Recogn. Site: Nb.BsrDI; Format: tl
ttggccacattagcattgcgcgctcgctcactcactcggccctggagaccaaaggtctccagactgccggcctctggccggcagggccgagtgagtgagcgagcgcgcaatgagggagtggccaa

SEQ ID NO: 72 - DNA, 137 bp, Synthetic construct
  source 1..137: mol_type = other DNA; note = source: AAV5; Recogn. Site: Nb.BsrDI; Format: bl
ctctccgcaatgtcgcgttcgctcgctcgctggctcgtttgggggggtggcagctcaaagagctgccagacgacggccctctggccgtcgcccccccaaacgagccagcgagcgagcgaacgcgacattgcggagag

SEQ ID NO: 73 - DNA, 137 bp, Synthetic construct
  source 1..137: mol_type = other DNA; note = source: AAV5; Recogn. Site: Nb.BsrDI; Format: tl
ctctcccccattgcgcgttcgctcgctcgctggctcgtttgggggggtggcagctcaaagagctgccagacgacggccctctggccgtcgcccccccaaacgagccagcgagcgagcgaacgcgcaatgggggagag

SEQ ID NO: 74 - DNA, 125 bp, Synthetic construct
  source 1..125: mol_type = other DNA; note = source: AAV7; Recogn. Site: Nb.BsrDI; Format: bl
ttggccactcccgcaatgcgcgctcgctcgctcggtggggcctgcggaccaaaggtccgcagacggcagagctctgctctgccggccccaccgagcgagcgagcgcgcattgcgggagtggccaa

SEQ ID NO: 75 - DNA, 125 bp, Synthetic construct
  source 1..125: mol_type = other DNA; note = source: AAV7; Recogn. Site: Nb.BsrDI; Format: tl
ttggccactccctcattgcgcgctcgctcgctcggtggggcctgcggaccaaaggtccgcagacggcagagctctgctctgccggccccaccgagcgagcgagcgcgcaatgagggagtggccaa

SEQ ID NO: 76 - DNA, 125 bp, Synthetic construct
  source 1..125: mol_type = other DNA; note = source: AAV1; Recogn. Site: Nb.BssSI; Format: bl
ttgcccacgagctctctgcgcgctcgctcgctcggtggggcctgcggaccaaaggtccgcagacggcagagctctgctctgccggccccaccgagcgagcgagcgcgcagagagctcgtgggcaa

SEQ ID NO: 77 - DNA, 125 bp, Synthetic construct
  source 1..125: mol_type = other DNA; note = source: AAV1; Recogn. Site: Nb.BssSI; Format: tl
ttgcccactccctcgtggcgcgctcgctcgctcggtggggcctgcggaccaaaggtccgcagacggcagagctctgctctgccggccccaccgagcgagcgagcgcgccacgagggagtgggcaa

SEQ ID NO: 78 - DNA, 125 bp, Synthetic construct
  source 1..125: mol_type = other DNA; note = source: AAV2; Recogn. Site: Nb.BssSI; Format: bl
ttggccacgagctctctgcgcgctcgctcgctcactgaggccgggcgaccaaaggtcgcccgacgcccgggctttgcccgggcggcctcagtgagcgagcgagcgcgcagagagctcgtggccaa

SEQ ID NO: 79 - DNA, 125 bp, Synthetic construct
  source 1..125: mol_type = other DNA; note = source: AAV2; Recogn. Site: Nb.BssSI; Format: tl
ttggccactccctcgtggcgcgctcgctcgctcactgaggccgggcgaccaaaggtcgcccgacgcccgggctttgcccgggcggcctcagtgagcgagcgagcgcgccacgagggagtggccaa

SEQ ID NO: 80 - DNA, 125 bp, Synthetic construct
  source 1..125: mol_type = other DNA; note = source: AAV3; Recogn. Site: Nb.BssSI; Format: bl
ttggccacgagctctatgcgcactcgctcgctcggtggggcctggcgaccaaaggtcgccagacggacgtgctttgcacgtccggccccaccgagcgagcgagtgcgcatagagctcgtggccaa

SEQ ID NO: 81 - DNA, 125 bp, Synthetic construct
  source 1..125: mol_type = other DNA; note = source: AAV3; Recogn. Site: Nb.BssSI; Format: tl
ttggccactccctcgtggcgcactcgctcgctcggtggggcctggcgaccaaaggtcgccagacggacgtgctttgcacgtccggccccaccgagcgagcgagtgcgccacgagggagtggccaa

SEQ ID NO: 82 - DNA, 125 bp, Synthetic construct
  source 1..125: mol_type = other DNA; note = source: AAV4 left; Recogn. Site: Nb.BssSI; Format: bl
ttggccacgagctctatgcgcgctcgctcactcactcggccctggagaccaaaggtctccagactgccggcctctggccggcagggccgagtgagtgagcgagcgcgcatagagctcgtggccaa

SEQ ID NO: 83 - DNA, 125 bp, Synthetic construct
  source 1..125: mol_type = other DNA; note = source: AAV4 left; Recogn. Site: Nb.BssSI; Format: tl
ttggccactccctcgtggcgcgctcgctcactcactcggccctggagaccaaaggtctccagactgccggcctctggccggcagggccgagtgagtgagcgagcgcgccacgagggagtggccaa

SEQ ID NO: 84 - DNA, 125 bp, Synthetic construct
  source 1..125: mol_type = other DNA; note = source: AAV4 right; Recogn. Site: Nb.BssSI; Format: bl
ttggccacgagagctatgcgcgctcgctcactcactcggccctggagaccaaaggtctccagactgccggcctctggccggcagggccgagtgagtgagcgagcgcgcatagagctcgtggccaa

SEQ ID NO: 85 - DNA, 125 bp, Synthetic construct
  source 1..125: mol_type = other DNA; note = source: AAV4 right; Recogn. Site: Nb.BssSI; Format: tl
ttggccacattctcgtggcgcgctcgctcactcactcggccctggagaccaaaggtctccagactgccggcctctggccggcagggccgagtgagtgagcgagcgcgccacgagggagtggccaa

SEQ ID NO: 86 - DNA, 137 bp, Synthetic construct
  source 1..137: mol_type = other DNA; note = source: AAV5; Recogn. Site: Nb.BssSI; Format: bl
ctcacgagcctgtcgcgttcgctcgctcgctggctcgtttgggggggtggcagctcaaagagctgccagacgacggccctctggccgtcgcccccccaaacgagccagcgagcgagcgaacgcgacaggctcgtgag

SEQ ID NO: 87 - DNA, 137 bp, Synthetic construct
  source 1..137: mol_type = other DNA; note = source: AAV5; Recogn. Site: Nb.BssSI; Format: tl
ctctccctcgtgtcgcgttcgctcgctcgctggctcgtttgggggggtggcagctcaaagagctgccagacgacggccctctggccgtcgcccccccaaacgagccagcgagcgagcgaacgcgacacgagggagag

SEQ ID NO: 88 - DNA, 125 bp, Synthetic construct
  source 1..125: mol_type = other DNA; note = source: AAV7; Recogn. Site: Nb.BssSI; Format: bl
ttggccacgagctctatgcgcgctcgctcgctcggtggggcctgcggaccaaaggtccgcagacggcagagctctgctctgccggccccaccgagcgagcgagcgcgcatagagctcgtggccaa

SEQ ID NO: 89 - DNA, 125 bp, Synthetic construct
  source 1..125: mol_type = other DNA; note = source: AAV7; Recogn. Site: Nb.BssSI; Format: tl
ttggccactccctcgtggcgcgctcgctcgctcggtggggcctgcggaccaaaggtccgcagacggcagagctctgctctgccggccccaccgagcgagcgagcgcgccacgagggagtggccaa

SEQ ID NO: 90 - DNA, 125 bp, Synthetic construct
  source 1..125: mol_type = other DNA; note = source: AAV1; Recogn. Site: Nb.BtsI; Format: bl
ttgcccactcccgcagtgcgcgctcgctcgctcggtggggcctgcggaccaaaggtccgcagacggcagagctctgctctgccggccccaccgagcgagcgagcgcgcactgcgggagtgggcaa

SEQ ID NO: 91 - DNA, 125 bp, Synthetic construct
  source 1..125: mol_type = other DNA; note = source: AAV1; Recogn. Site: Nb.BtsI; Format: tl
ttgcccactccctcactgcgcgctcgctcgctcggtggggcctgcggaccaaaggtccgcagacggcagagctctgctctgccggccccaccgagcgagcgagcgcgcagtgagggagtgggcaa

SEQ ID NO: 92 - DNA, 125 bp, Synthetic construct
  source 1..125: mol_type = other DNA; note = source: AAV2; Recogn. Site: Nb.BtsI; Format: bl
ttggccactcccgcagtgcgcgctcgctcgctcactgaggccgggcgaccaaaggtcgcccgacgcccgggctttgcccgggcggcctcagtgagcgagcgagcgcgcactgcgggagtggccaa

SEQ ID NO: 93 - DNA, 125 bp, Synthetic construct
  source 1..125: mol_type = other DNA; note = source: AAV2; Recogn. Site: Nb.BtsI; Format: tl
ttggccactccctcactgcgcgctcgctcgctcactgaggccgggcgaccaaaggtcgcccgacgcccgggctttgcccgggcggcctcagtgagcgagcgagcgcgcagtgagggagtggccaa

SEQ ID NO: 94 - DNA, 125 bp, Synthetic construct
  source 1..125: mol_type = other DNA; note = source: AAV3; Recogn. Site: Nb.BtsI; Format: bl
ttggccactcccgcagtgcgcactcgctcgctcggtggggcctggcgaccaaaggtcgccagacggacgtgctttgcacgtccggccccaccgagcgagcgagtgcgcactgcgggagtggccaa

SEQ ID NO: 95 - DNA, 125 bp, Synthetic construct
  source 1..125: mol_type = other DNA; note = source: AAV3; Recogn. Site: Nb.BtsI; Format: tl
ttggccactccctcactgcgcactcgctcgctcggtggggcctggcgaccaaaggtcgccagacggacgtgctttgcacgtccggccccaccgagcgagcgagtgcgcagtgagggagtggccaa

SEQ ID NO: 96 - DNA, 125 bp, Synthetic construct
  source 1..125: mol_type = other DNA; note = source: AAV4 left; Recogn. Site: Nb.BtsI; Format: bl
ttggccactcccgcagtgcgcgctcgctcactcactcggccctggagaccaaaggtctccagactgccggcctctggccggcagggccgagtgagtgagcgagcgcgcactgcgggagtggccaa

SEQ ID NO: 97 - DNA, 125 bp, Synthetic construct
  source 1..125: mol_type = other DNA; note = source: AAV4 left; Recogn. Site: Nb.BtsI; Format: tl
ttggccactccctcactgcgcgctcgctcactcactcggccctggagaccaaaggtctccagactgccggcctctggccggcagggccgagtgagtgagcgagcgcgcagtgagggagtggccaa

SEQ ID NO: 98 - DNA, 125 bp, Synthetic construct
  source 1..125: mol_type = other DNA; note = source: AAV4 right; Recogn. Site: Nb.BtsI; Format: bl
ttggccacattagcagtgcgcgctcgctcactcactcggccctggagaccaaaggtctccagactgccggcctctggccggcagggccgagtgagtgagcgagcgcgcactgcgggagtggccaa

SEQ ID NO: 99 - DNA, 125 bp, Synthetic construct
  source 1..125: mol_type = other DNA; note = source: AAV4 right; Recogn. Site: Nb.BtsI; Format: tl
ttggccacattagcactgcgcgctcgctcactcactcggccctggagaccaaaggtctccagactgccggcctctggccggcagggccgagtgagtgagcgagcgcgcagtgagggagtggccaa

SEQ ID NO: 100 - DNA, 137 bp, Synthetic construct
  source 1..137: mol_type = other DNA; note = source: AAV5; Recogn. Site: Nb.BtsI; Format: bl
ctctccgcagtgtcgcgttcgctcgctcgctggctcgtttgggggggtggcagctcaaagagctgccagacgacggccctctggccgtcgcccccccaaacgagccagcgagcgagcgaacgcgacactgcggagag

SEQ ID NO: 101 - DNA, 137 bp, Synthetic construct
  source 1..137: mol_type = other DNA; note = source: AAV5; Recogn. Site: Nb.BtsI; Format: tl
ctctccccactgccgcgttcgctcgctcgctggctcgtttgggggggtggcagctcaaagagctgccagacgacggccctctggccgtcgcccccccaaacgagccagcgagcgagcgaacgcggcagtggggagag

SEQ ID NO: 102 - DNA, 125 bp, Synthetic construct
  source 1..125: mol_type = other DNA; note = source: AAV7; Recogn. Site: Nb.BtsI; Format: bl
ttggccactcccgcagtgcgcgctcgctcgctcggtggggcctgcggaccaaaggtccgcagacggcagagctctgctctgccggccccaccgagcgagcgagcgcgcactgcgggagtggccaa

SEQ ID NO: 103 - DNA, 125 bp, Synthetic construct
  source 1..125: mol_type = other DNA; note = source: AAV7; Recogn. Site: Nb.BtsI; Format: tl
ttggccactccctcactgcgcgctcgctcgctcggtggggcctgcggaccaaaggtccgcagacggcagagctctgctctgccggccccaccgagcgagcgagcgcgcagtgagggagtggccaa

SEQ ID NO: 104 - DNA, 125 bp, Synthetic construct
  source 1..125: mol_type = other DNA; note = source: AAV1; Recogn. Site: Nt.AlwI; Format: bl
ttgcccactccctcgatccgcgctcgctcgctcggtggggcctgcggaccaaaggtccgcagacggcagagctctgctctgccggccccaccgagcgagcgagcgcggatcgagggagtgggcaa

SEQ ID NO: 105 - DNA, 125 bp, Synthetic construct
  source 1..125: mol_type = other DNA; note = source: AAV1; Recogn. Site: Nt.AlwI; Format: tl
ttgcccactggatctctgcgcgctcgctcgctcggtggggcctgcggaccaaaggtccgcagacggcagagctctgctctgccggccccaccgagcgagcgagcgcgcagagatccagtgggcaa

SEQ ID NO: 106 - DNA, 125 bp, Synthetic construct
  source 1..125: mol_type = other DNA; note = source: AAV2; Recogn. Site: Nt.AlwI; Format: bl
ttggccactccctcgatccgcgctcgctcgctcactgaggccgggcgaccaaaggtcgcccgacgcccgggctttgcccgggcggcctcagtgagcgagcgagcgcggatcgagggagtggccaa

SEQ ID NO: 107 - DNA, 125 bp, Synthetic construct
  source 1..125: mol_type = other DNA; note = source: AAV2; Recogn. Site: Nt.AlwI; Format: tl
ttggccactggatctctgcgcgctcgctcgctcactgaggccgggcgaccaaaggtcgcccgacgcccgggctttgcccgggcggcctcagtgagcgagcgagcgcgcagagatccagtggccaa

SEQ ID NO: 108 - DNA, 125 bp, Synthetic construct
  source 1..125: mol_type = other DNA; note = source: AAV3; Recogn. Site: Nt.AlwI; Format: bl
ttggccactccctcgatccgcactcgctcgctcggtggggcctggcgaccaaaggtcgccagacggacgtgctttgcacgtccggccccaccgagcgagcgagtgcggatcgagggagtggccaa

SEQ ID NO: 109 - DNA, 125 bp, Synthetic construct
  source 1..125: mol_type = other DNA; note = source: AAV3; Recogn. Site: Nt.AlwI; Format: tl
ttggccactggatctatgcgcactcgctcgctcggtggggcctggcgaccaaaggtcgccagacggacgtgctttgcacgtccggccccaccgagcgagcgagtgcgcatagatccagtggccaa

SEQ ID NO: 110 - DNA, 125 bp, Synthetic construct
  source 1..125: mol_type = other DNA; note = source: AAV4 left; Recogn. Site: Nt.AlwI; Format: bl
ttggccactccctcgatccgcgctcgctcactcactcggccctggagaccaaaggtctccagactgccggcctctggccggcagggccgagtgagtgagcgagcgcggatcgagggagtggccaa

SEQ ID NO: 111 - DNA, 125 bp, Synthetic construct
  source 1..125: mol_type = other DNA; note = source: AAV4 left; Recogn. Site: Nt.AlwI; Format: tl
ttggccactggatctatgcgcgctcgctcactcactcggccctggagaccaaaggtctccagactgccggcctctggccggcagggccgagtgagtgagcgagcgcgcatagatccagtggccaa

SEQ ID NO: 112 - DNA, 125 bp, Synthetic construct
  source 1..125: mol_type = other DNA; note = source: AAV4 right; Recogn. Site: Nt.AlwI; Format: bl
ttggccacattagcgatccgcgctcgctcactcactcggccctggagaccaaaggtctccagactgccggcctctggccggcagggccgagtgagtgagcgagcgcggatcgagggagtggccaa

SEQ ID NO: 113 - DNA, 125 bp, Synthetic construct
  source 1..125: mol_type = other DNA; note = source: AAV4 right; Recogn. Site: Nt.AlwI; Format: tl
ttggccacaggatctatgcgcgctcgctcactcactcggccctggagaccaaaggtctccagactgccggcctctggccggcagggccgagtgagtgagcgagcgcgcatagatccagtggccaa

SEQ ID NO: 114 - DNA, 137 bp, Synthetic construct
  source 1..137: mol_type = other DNA; note = source: AAV5; Recogn. Site: Nt.AlwI; Format: bl
ctctcccccctgtcgcgatccctcgctcgctggctcgtttgggggggtggcagctcaaagagctgccagacgacggccctctggccgtcgcccccccaaacgagccagcgagcgagggatcgcgacaggggggagag

SEQ ID NO: 115 - DNA, 137 bp, Synthetic construct
  source 1..137: mol_type = other DNA; note = source: AAV5; Recogn. Site: Nt.AlwI; Format: tl
ctctcccccggatcgcgttcgctcgctcgctggctcgtttgggggggtggcagctcaaagagctgccagacgacggccctctggccgtcgcccccccaaacgagccagcgagcgagcgaacgcgatccgggggagag

SEQ ID NO: 116 - DNA, 125 bp, Synthetic construct
  source 1..125: mol_type = other DNA; note = source: AAV7; Recogn. Site: Nt.AlwI; Format: bl
ttggccactccctcgatccgcgctcgctcgctcggtggggcctgcggaccaaaggtccgcagacggcagagctctgctctgccggccccaccgagcgagcgagcgcggatcgagggagtggccaa

SEQ ID NO: 117 - DNA, 125 bp, Synthetic construct
  source 1..125: mol_type = other DNA; note = source: AAV7; Recogn. Site: Nt.AlwI; Format: tl
ttggccactggatctatgcgcgctcgctcgctcggtggggcctgcggaccaaaggtccgcagacggcagagctctgctctgccggccccaccgagcgagcgagcgcgcatagatccagtggccaa

SEQ ID NO: 118 - DNA, 125 bp, Synthetic construct; identical to SEQ ID NO: 35
  source 1..125: mol_type = other DNA; note = source: AAV1; Recogn. Site: Nt.BbvCI; Format: bl

SEQ ID NO: 119 - DNA, 125 bp, Synthetic construct; identical to SEQ ID NO: 34
  source 1..125: mol_type = other DNA; note = source: AAV1; Recogn. Site: Nt.BbvCI; Format: tl

SEQ ID NO: 120 - DNA, 125 bp, Synthetic construct; identical to SEQ ID NO: 37
  source 1..125: mol_type = other DNA; note = source: AAV2; Recogn. Site: Nt.BbvCI; Format: bl

SEQ ID NO: 121 - DNA, 125 bp, Synthetic construct; identical to SEQ ID NO: 36
  source 1..125: mol_type = other DNA; note = source: AAV2; Recogn. Site: Nt.BbvCI; Format: tl

SEQ ID NO: 122 - DNA, 125 bp, Synthetic construct; identical to SEQ ID NO: 39
  source 1..125: mol_type = other DNA; note = source: AAV3; Recogn. Site: Nt.BbvCI; Format: bl

SEQ ID NO: 123 - DNA, 125 bp, Synthetic construct; identical to SEQ ID NO: 38
  source 1..125: mol_type = other DNA; note = source: AAV3; Recogn. Site: Nt.BbvCI; Format: tl

SEQ ID NO: 124 - DNA, 125 bp, Synthetic construct; identical to SEQ ID NO: 41
  source 1..125: mol_type = other DNA; note = source: AAV4 left; Recogn. Site: Nt.BbvCI; Format: bl

SEQ ID NO: 125 - DNA, 125 bp, Synthetic construct; identical to SEQ ID NO: 40
  source 1..125: mol_type = other DNA; note = source: AAV4 left; Recogn. Site: Nt.BbvCI; Format: tl

SEQ ID NO: 126 - DNA, 125 bp, Synthetic construct; identical to SEQ ID NO: 43
  source 1..125: mol_type = other DNA; note = source: AAV4 right; Recogn. Site: Nt.BbvCI; Format: bl

SEQ ID NO: 127 - DNA, 125 bp, Synthetic construct; identical to SEQ ID NO: 42
  source 1..125: mol_type = other DNA; note = source: AAV4 right; Recogn. Site: Nt.BbvCI; Format: tl

SEQ ID NO: 128 - DNA, 137 bp, Synthetic construct; identical to SEQ ID NO: 45
  source 1..137: mol_type = other DNA; note = source: AAV5; Recogn. Site: Nt.BbvCI; Format: bl

SEQ ID NO: 129 - DNA, 137 bp, Synthetic construct; identical to SEQ ID NO: 44
  source 1..137: mol_type = other DNA; note = source: AAV5; Recogn. Site: Nt.BbvCI; Format: tl

SEQ ID NO: 130 - DNA, 125 bp, Synthetic construct; identical to SEQ ID NO: 47
  source 1..125: mol_type = other DNA; note = source: AAV7; Recogn. Site: Nt.BbvCI; Format: bl

SEQ ID NO: 131 - DNA, 125 bp, Synthetic construct; identical to SEQ ID NO: 46
  source 1..125: mol_type = other DNA; note = source: AAV7; Recogn. Site: Nt.BbvCI; Format: tl

SEQ ID NO: 132 - DNA, 125 bp, Synthetic construct
  source 1..125: mol_type = other DNA; note = source: AAV1; Recogn. Site: Nt.BsmAI; Format: bl
ttgcccactgagactctgcgcgctcgctcgctcggtggggcctgcggaccaaaggtccgcagacggcagagctctgctctgccggccccaccgagcgagcgagcgcgcagagtctcagtgggcaa

SEQ ID NO: 133 - DNA, 125 bp, Synthetic construct
  source 1..125: mol_type = other DNA; note = source: AAV1; Recogn. Site: Nt.BsmAI; Format: tl
ttgcccactccgtctctgcgcgctcgctcgctcggtggggcctgcggaccaaaggtccgcagacggcagagctctgctctgccggccccaccgagcgagcgagcgcgcagagacggagtgggcaa

SEQ ID NO: 134 - DNA, 125 bp, Synthetic construct
  source 1..125: mol_type = other DNA; note = source: AAV2; Recogn. Site: Nt.BsmAI; Format: bl
ttggccactgagactctgcgcgctcgctcgctcactgaggccgggcgaccaaaggtcgcccgacgcccgggctttgcccgggcggcctcagtgagcgagcgagcgcgcagagtctcagtggccaa

SEQ ID NO: 135 - DNA, 125 bp, Synthetic construct
  source 1..125: mol_type = other DNA; note = source: AAV2; Recogn. Site: Nt.BsmAI; Format: tl
ttggccactccgtctctgcgcgctcgctcgctcactgaggccgggcgaccaaaggtcgcccgacgcccgggctttgcccgggcggcctcagtgagcgagcgagcgcgcagagacggagtggccaa

SEQ ID NO: 136 - DNA, 125 bp, Synthetic construct
  source 1..125: mol_type = other DNA; note = source: AAV3; Recogn. Site: Nt.BsmAI; Format: bl
ttggccactgagactatgcgcactcgctcgctcggtggggcctggcgaccaaaggtcgccagacggacgtgctttgcacgtccggccccaccgagcgagcgagtgcgcatagtctcagtggccaa

SEQ ID NO: 137 - DNA, 125 bp, Synthetic construct
  source 1..125: mol_type = other DNA; note = source: AAV3; Recogn. Site: Nt.BsmAI; Format: tl
ttggccactccgtctctgcgcactcgctcgctcggtggggcctggcgaccaaaggtcgccagacggacgtgctttgcacgtccggccccaccgagcgagcgagtgcgcagagacggagtggccaa

SEQ ID NO: 138 - DNA, 125 bp, Synthetic construct
  source 1..125: mol_type = other DNA; note = source: AAV4 left; Recogn. Site: Nt.BsmAI; Format: bl
ttggccactgagactatgcgcgctcgctcactcactcggccctggagaccaaaggtctccagactgccggcctctggccggcagggccgagtgagtgagcgagcgcgcatagtctcagtggccaa

SEQ ID NO: 139 - DNA, 125 bp, Synthetic construct
  source 1..125: mol_type = other DNA; note = source: AAV4 left; Recogn. Site: Nt.BsmAI; Format: tl
ttggccactccgtctctgcgcgctcgctcactcactcggccctggagaccaaaggtctccagactgccggcctctggccggcagggccgagtgagtgagcgagcgcgcagagacggagtggccaa

SEQ ID NO: 140 - DNA, 125 bp, Synthetic construct
  source 1..125: mol_type = other DNA; note = source: AAV4 right; Recogn. Site: Nt.BsmAI; Format: bl
ttggccacagagactatgcgcgctcgctcactcactcggccctggagaccaaaggtctccagactgccggcctctggccggcagggccgagtgagtgagcgagcgcgcatagtctcagtggccaa

SEQ ID NO: 141 - DNA, 125 bp, Synthetic construct
  source 1..125: mol_type = other DNA; note = source: AAV4 right; Recogn. Site: Nt.BsmAI; Format: tl
ttggccacattgtctctgcgcgctcgctcactcactcggccctggagaccaaaggtctccagactgccggcctctggccggcagggccgagtgagtgagcgagcgcgcagagacggagtggccaa

SEQ ID NO: 142 - DNA, 137 bp, Synthetic construct
  source 1..137: mol_type = other DNA; note = source: AAV5; Recogn. Site: Nt.BsmAI; Format: bl
ctctcccccgagacgcgttcgctcgctcgctggctcgtttgggggggtggcagctcaaagagctgccagacgacggccctctggccgtcgcccccccaaacgagccagcgagcgagcgaacgcgtctcgggggagag

SEQ ID NO: 143 - DNA, 137 bp, Synthetic construct
  source 1..137: mol_type = other DNA; note = source: AAV5; Recogn. Site: Nt.BsmAI; Format: tl
ctctcccccgtctcgcgttcgctcgctcgctggctcgtttgggggggtggcagctcaaagagctgccagacgacggccctctggccgtcgcccccccaaacgagccagcgagcgagcgaacgcgagacgggggagag

SEQ ID NO: 144 - DNA, 125 bp, Synthetic construct
  source 1..125: mol_type = other DNA; note = source: AAV7; Recogn. Site: Nt.BsmAI; Format: bl
ttggccactgagactatgcgcgctcgctcgctcggtggggcctgcggaccaaaggtccgcagacggcagagctctgctctgccggccccaccgagcgagcgagcgcgcatagtctcagtggccaa

SEQ ID NO: 145 - DNA, 125 bp, Synthetic construct
  source 1..125: mol_type = other DNA; note = source: AAV7; Recogn. Site: Nt.BsmAI; Format: tl
ttggccactccgtctctgcgcgctcgctcgctcggtggggcctgcggaccaaaggtccgcagacggcagagctctgctctgccggccccaccgagcgagcgagcgcgcagagacggagtggccaa

SEQ ID NO: 146 - DNA, 125 bp, Synthetic construct
  source 1..125: mol_type = other DNA; note = source: AAV1; Recogn. Site: Nt.BspQI; Format: bl
ttgcccactcccgaagagcgcgctcgctcgctcggtggggcctgcggaccaaaggtccgcagacggcagagctctgctctgccggccccaccgagcgagcgagcgcgctcttcgggagtgggcaa

SEQ ID NO: 147 - DNA, 125 bp, Synthetic construct
  source 1..125: mol_type = other DNA; note = source: AAV1; Recogn. Site: Nt.BspQI; Format: tl
ttgcccactcccgctcttcgcgctcgctcgctcggtggggcctgcggaccaaaggtccgcagacggcagagctctgctctgccggccccaccgagcgagcgagcgcgaagagcgggagtgggcaa

SEQ ID NO: 148 - DNA, 125 bp, Synthetic construct
  source 1..125: mol_type = other DNA; note = source: AAV2; Recogn. Site: Nt.BspQI; Format: bl
ttggccactcccgaagagcgcgctcgctcgctcactgaggccgggcgaccaaaggtcgcccgacgcccgggctttgcccgggcggcctcagtgagcgagcgagcgcgctcttcgggagtggccaa

SEQ ID NO: 149 - DNA, 125 bp, Synthetic construct
  source 1..125: mol_type = other DNA; note = source: AAV2; Recogn. Site: Nt.BspQI; Format: tl
ttggccactcccgctcttcgcgctcgctcgctcactgaggccgggcgaccaaaggtcgcccgacgcccgggctttgcccgggcggcctcagtgagcgagcgagcgcgaagagcgggagtggccaa

SEQ ID NO: 150 - DNA, 125 bp, Synthetic construct
  source 1..125: mol_type = other DNA; note = source: AAV3; Recogn. Site: Nt.BspQI; Format: bl
ttggccactcccgaagagcgcactcgctcgctcggtggggcctggcgaccaaaggtcgccagacggacgtgctttgcacgtccggccccaccgagcgagcgagtgcgctcttcgggagtggccaa

SEQ ID NO: 151 - DNA, 125 bp, Synthetic construct
  source 1..125: mol_type = other DNA; note = source: AAV3; Recogn. Site: Nt.BspQI; Format: tl
ttggccactcccgctcttcgcactcgctcgctcggtggggcctggcgaccaaaggtcgccagacggacgtgctttgcacgtccggccccaccgagcgagcgagtgcgaagagcgggagtggccaa

SEQ ID NO: 152 - DNA, 125 bp, Synthetic construct
  source 1..125: mol_type = other DNA; note = source: AAV4 left; Recogn. Site: Nt.BspQI; Format: bl
ttggccactcccgaagagcgcgctcgctcactcactcggccctggagaccaaaggtctccagactgccggcctctggccggcagggccgagtgagtgagcgagcgcgctcttcgggagtggccaa

SEQ ID NO: 153 - DNA, 125 bp, Synthetic construct
  source 1..125: mol_type = other DNA; note = source: AAV4 left; Recogn. Site: Nt.BspQI; Format: tl
ttggccactcccgctcttcgcgctcgctcactcactcggccctggagaccaaaggtctccagactgccggcctctggccggcagggccgagtgagtgagcgagcgcgaagagcgggagtggccaa

SEQ ID NO: 154 - DNA, 125 bp, Synthetic construct
  source 1..125: mol_type = other DNA; note = source: AAV4 right; Recogn. Site: Nt.BspQI; Format: bl
ttggccacattagaagagcgcgctcgctcactcactcggccctggagaccaaaggtctccagactgccggcctctggccggcagggccgagtgagtgagcgagcgcgctcttcgggagtggccaa

SEQ ID NO: 155 - DNA, 125 bp, Synthetic construct
  source 1..125: mol_type = other DNA; note = source: AAV4 right; Recogn. Site: Nt.BspQI; Format: tl
ttggccacattagctcttcgcgctcgctcactcactcggccctggagaccaaaggtctccagactgccggcctctggccggcagggccgagtgagtgagcgagcgcgaagagcgggagtggccaa

SEQ ID NO: 156 - DNA, 137 bp, Synthetic construct
  source 1..137: mol_type = other DNA; note = source: AAV5; Recogn. Site: Nt.BspQI; Format: bl
ctctcccgaagagcgcgttcgctcgctcgctggctcgtttgggggggtggcagctcaaagagctgccagacgacggccctctggccgtcgcccccccaaacgagccagcgagcgagcgaacgcgctcttcgggagag

SEQ ID NO: 157 - DNA, 137 bp, Synthetic construct
  source 1..137: mol_type = other DNA; note = source: AAV5; Recogn. Site: Nt.BspQI; Format: tl
ctctcccgctcttcgcgttcgctcgctcgctggctcgtttgggggggtggcagctcaaagagctgccagacgacggccctctggccgtcgcccccccaaacgagccagcgagcgagcgaacgcgaagagcgggagag

SEQ ID NO: 158 - DNA, 125 bp, Synthetic construct
  source 1..125: mol_type = other DNA; note = source: AAV7; Recogn. Site: Nt.BspQI; Format: bl
ttggccactcccgaagagcgcgctcgctcgctcggtggggcctgcggaccaaaggtccgcagacggcagagctctgctctgccggccccaccgagcgagcgagcgcgctcttcgggagtggccaa

SEQ ID NO: 159 - DNA, 125 bp, Synthetic construct
  source 1..125: mol_type = other DNA; note = source: AAV7; Recogn. Site: Nt.BspQI; Format: tl
ttggccactcccgctcttcgcgctcgctcgctcggtggggcctgcggaccaaaggtccgcagacggcagagctctgctctgccggccccaccgagcgagcgagcgcgaagagcgggagtggccaa

SEQ ID NO: 160 - DNA, 127 bp, Synthetic construct
  source 1..127: mol_type = other DNA; note = source: AAV1; Recogn. Site: Nt.BstNBI; Format: bl
ttgcccactccctctctgcgcgactcgctcgctcggtggggcctgcggaccaaaggtccgcagacggcagagctctgctctgccggccccaccgagcgagcgagtcgcgcagagagggagtgggcaa

SEQ ID NO: 161 - DNA, 125 bp, Synthetic construct
  source 1..125: mol_type = other DNA; note = source: AAV1; Recogn. Site: Nt.BstNBI; Format: tl
ttgccgagtccctctctgcgcgctcgctcgctcggtggggcctgcggaccaaaggtccgcagacggcagagctctgctctgccggccccaccgagcgagcgagcgcgcagagagggactcggcaa

SEQ ID NO: 162 - DNA, 127 bp, Synthetic construct
  source 1..127: mol_type = other DNA; note = source: AAV2; Recogn. Site: Nt.BstNBI; Format: bl
ttggccactccctctctgcgcgactcgctcgctcactgaggccgggcgaccaaaggtcgcccgacgcccgggctttgcccgggcggcctcagtgagcgagcgagtcgcgcagagagggagtggccaa

SEQ ID NO: 163 - DNA, 125 bp, Synthetic construct
  source 1..125: mol_type = other DNA; note = source: AAV2; Recogn. Site: Nt.BstNBI; Format: tl
ttggcgagtccctctctgcgcgctcgctcgctcactgaggccgggcgaccaaaggtcgcccgacgcccgggctttgcccgggcggcctcagtgagcgagcgagcgcgcagagagggactcgccaa

SEQ ID NO: 164 - DNA, 127 bp, Synthetic construct
  source 1..127: mol_type = other DNA; note = source: AAV3; Recogn. Site: Nt.BstNBI; Format: bl
ttggccactccctctatgcgcgactcgctcgctcggtggggcctggcgaccaaaggtcgccagacggacgtgctttgcacgtccggccccaccgagcgagcgagtcgcgcatagagggagtggccaa

SEQ ID NO: 165 - DNA, 125 bp, Synthetic construct
  source 1..125: mol_type = other DNA; note = source: AAV3; Recogn. Site: Nt.BstNBI; Format: tl
ttggcgagtccctctatgcgcactcgctcgctcggtggggcctggcgaccaaaggtcgccagacggacgtgctttgcacgtccggccccaccgagcgagcgagtgcgcatagagggactcgccaa

SEQ ID NO: 166 - DNA, 127 bp, Synthetic construct
  source 1..127: mol_type = other DNA; note = source: AAV4 left; Recogn. Site: Nt.BstNBI; Format: bl
ttggccactccctctatgcgcgactcgctcactcactcggccctggagaccaaaggtctccagactgccggcctctggccggcagggccgagtgagtgagcgagtcgcgcatagagggagtggccaa

SEQ ID NO: 167 - DNA, 125 bp, Synthetic construct
  source 1..125: mol_type = other DNA; note = source: AAV4 left; Recogn. Site: Nt.BstNBI; Format: tl
ttggcgagtccctctatgcgcgctcgctcactcactcggccctggagaccaaaggtctccagactgccggcctctggccggcagggccgagtgagtgagcgagcgcgcatagagggactcgccaa

SEQ ID NO: 168 - DNA, 127 bp, Synthetic construct
  source 1..127: mol_type = other DNA; note = source: AAV4 right; Recogn. Site: Nt.BstNBI; Format: bl
ttggccacattagctatgcgcgactcgctcactcactcggccctggagaccaaaggtctccagactgccggcctctggccggcagggccgagtgagtgagcgagtcgcgcatagagggagtggccaa

SEQ ID NO: 169 - DNA, 125 bp, Synthetic construct
  source 1..125: mol_type = other DNA; note = source: AAV4 right; Recogn. Site: Nt.BstNBI; Format: tl
ttggccagagtcgctatgcgcgctcgctcactcactcggccctggagaccaaaggtctccagactgccggcctctggccggcagggccgagtgagtgagcgagcgcgcatagagactctggccaa

SEQ ID NO: 170 - DNA, 139 bp, Synthetic construct
  source 1..139: mol_type = other DNA; note = source: AAV5; Recogn. Site: Nt.BstNBI; Format: bl
ctctcccccctgtcgcgactcgctcgctcgctggctcgtttgggggggtggcagctcaaagagctgccagacgacggccctctggccgtcgcccccccaaacgagccagcgagcgagcgagtcgcgacaggggggagag

SEQ ID NO: 171 - DNA, 137 bp, Synthetic construct
  source 1..137: mol_type = other DNA; note = source: AAV5; Recogn. Site: Nt.BstNBI; Format: tl
ctctcccccgagtcgcgttcgctcgctcgctggctcgtttgggggggtggcagctcaaagagctgccagacgacggccctctggccgtcgcccccccaaacgagccagcgagcgagcgaacgcgactcgggggagag

SEQ ID NO: 172 - DNA, 127 bp, Synthetic construct
  source 1..127: mol_type = other DNA; note = source: AAV7; Recogn. Site: Nt.BstNBI; Format: bl
ttggccactccctctatgcgcgactcgctcgctcggtggggcctgcggaccaaaggtccgcagacggcagagctctgctctgccggccccaccgagcgagcgagtcgcgcatagagggagtggccaa

SEQ ID NO: 173 - DNA, 125 bp, Synthetic construct
  source 1..125: mol_type = other DNA; note = source: AAV7; Recogn. Site: Nt.BstNBI; Format: tl
ttggcgagtccctctatgcgcgctcgctcgctcggtggggcctgcggaccaaaggtccgcagacggcagagctctgctctgccggccccaccgagcgagcgagcgcgcatagagggactcgccaa

SEQ ID NO: 174 - DNA, 99 bp, Synthetic construct
  source 1..99: mol_type = other DNA; note = wt_AAV1
aacgggtgagggagagacgcgcgagcgagcgagccaccccggacgcctggtttccaggcgtctgccgtctcgagacgagacggccggggtggctcgctc

SEQ ID NO: 175 - DNA, 99 bp, Synthetic construct
  source 1..99: mol_type = other DNA; note = AAV1_Nb.BbvCI_BL
aacgggtgagggggagtcgcgcgagcgagcgagccaccccggacgcctggtttccaggcgtctgccgtctcgagacgagacggccggggtggctcgctc

SEQ ID NO: 176 - DNA, 99 bp, Synthetic construct
  source 1..99: mol_type = other DNA; note = AAV1_Nb.BbvCI_TL
aacgggtgagggcgactcccgcgagcgagcgagccaccccggacgcctggtttccaggcgtctgccgtctcgagacgagacggccggggtggctcgctc

SEQ ID NO: 177 - DNA, 99 bp, Synthetic construct
  source 1..99: mol_type = other DNA; note = AAV1_Nb.BsmI_BL
aacgggtgagggacttacgcgcgagcgagcgagccaccccggacgcctggtttccaggcgtctgccgtctcgagacgagacggccggggtggctcgctc

SEQ ID NO: 178 - DNA, 99 bp, Synthetic construct
  source 1..99: mol_type = other DNA; note = AAV1_Nb.BsmI_TL
aacgggtgagggagagacgcgtaagcgagcgagccaccccggacgcctggtttccaggcgtctgccgtctcgagacgagacggccggggtggctcgctc

SEQ ID NO: 179 - DNA, 99 bp, Synthetic construct
  source 1..99: mol_type = other DNA; note = AAV1_Nb.BsrDI_BL
aacgggtgagggcgttacgcgcgagcgagcgagccaccccggacgcctggtttccaggcgtctgccgtctcgagacgagacggccggggtggctcgctc

SEQ ID NO: 180 - DNA, 99 bp, Synthetic construct
  source 1..99: mol_type = other DNA; note = AAV1_Nb.BsrDI_TL
aacgggtgagggagtaacgcgcgagcgagcgagccaccccggacgcctggtttccaggcgtctgccgtctcgagacgagacggccggggtggctcgctc

SEQ ID NO: 181 - DNA, 99 bp, Synthetic construct
  source 1..99: mol_type = other DNA; note = AAV1_Nb.BssSI_BL
aacgggtgctcgagagacgcgcgagcgagcgagccaccccggacgcctggtttccaggcgtctgccgtctcgagacgagacggccggggtggctcgctc

SEQ ID NO: 182 - DNA, 99 bp, Synthetic construct
  source 1..99: mol_type = other DNA; note = AAV1_Nb.BssSI_TL
aacgggtgagggagcaccgcgcgagcgagcgagccaccccggacgcctggtttccaggcgtctgccgtctcgagacgagacggccggggtggctcgctc

SEQ ID NO: 183 - DNA, 99 bp, Synthetic construct
  source 1..99: mol_type = other DNA; note = AAV1_Nb.BtsI_BL
aacgggtgagggcgtcacgcgcgagcgagcgagccaccccggacgcctggtttccaggcgtctgccgtctcgagacgagacggccggggtggctcgctc

SEQ ID NO: 184 - DNA, 99 bp, Synthetic construct
  source 1..99: mol_type = other DNA; note = AAV1_Nb.BtsI_TL
aacgggtgagggagtgacgcgcgagcgagcgagccaccccggacgcctggtttccaggcgtctgccgtctcgagacgagacggccggggtggctcgctc

SEQ ID NO: 185 - DNA, 99 bp, Synthetic construct
  source 1..99: mol_type = other DNA; note = AAV1_Nt.AlwI_BL
aacgggtgagggagctaggcgcgagcgagcgagccaccccggacgcctggtttccaggcgtctgccgtctcgagacgagacggccggggtggctcgctc

SEQ ID NO: 186 - DNA, 99 bp, Synthetic construct
  source 1..99: mol_type = other DNA; note = AAV1_Nt.AlwI_BL
aacgggtgacctagagacgcgcgagcgagcgagccaccccggacgcctggtttccaggcgtctgccgtctcgagacgagacggccggggtggctcgctc

SEQ ID NO: 187 - DNA, 99 bp, Synthetic construct; identical to SEQ ID NO: 176
  source 1..99: mol_type = other DNA; note = AAV1_Nt.BbvCI_TL

SEQ ID NO: 188 - DNA, 99 bp, Synthetic construct; identical to SEQ ID NO: 175
  source 1..99: mol_type = other DNA; note = AAV1_Nt.BbvCI_BL

SEQ ID NO: 189 - DNA, 99 bp, Synthetic construct
  source 1..99: mol_type = other DNA; note = AAV1_Nt.BsmAI_TL
aacgggtgactctgagacgcgcgagcgagcgagccaccccggacgcctggtttccaggcgtctgccgtctcgagacgagacggccggggtggctcgctc

SEQ ID NO: 190 - DNA, 99 bp, Synthetic construct
  source 1..99: mol_type = other DNA; note = AAV1_Nt.BsmAI_BL
aacgggtgaggcagagacgcgcgagcgagcgagccaccccggacgcctggtttccaggcgtctgccgtctcgagacgagacggccggggtggctcgctc

SEQ ID NO: 191 - DNA, 99 bp, Synthetic construct
  source 1..99: mol_type = other DNA; note = AAV1_Nt.BspQI_TL
aacgggtgagggcttctcgcgcgagcgagcgagccaccccggacgcctggtttccaggcgtctgccgtctcgagacgagacggccggggtggctcgctc

SEQ ID NO: 192 - DNA, 99 bp, Synthetic construct
  source 1..99: mol_type = other DNA; note = AAV1_Nt.BspQI_BL
aacgggtgagggcgagaagcgcgagcgagcgagccaccccggacgcctggtttccaggcgtctgccgtctcgagacgagacggccggggtggctcgctc

SEQ ID NO: 193 - DNA, 100 bp, Synthetic construct
  source 1..100: mol_type = other DNA; note = AAV1_Nt.BstNBI_TL
aacgggtgagggagagacgcgctgagcgagcgagccaccccggacgcctggtttccaggcgtctgccgtctcgagacgagacggccggggtggctcgctc

SEQ ID NO: 194 - DNA, 99 bp, Synthetic construct
  source 1..99: mol_type = other DNA; note = AAV1_Nt.BstNBI_BL
aacggctcagggagagacgcgcgagcgagcgagccaccccggacgcctggtttccaggcgtctgccgtctcgagacgagacggccggggtggctcgctc

SEQ ID NO: 195 - DNA, 99 bp, Synthetic construct
  source 1..99: mol_type = other DNA; note = wt_AAV2
aaccggtgagggagagacgcgcgagcgagcgagtgactccggcccgctggtttccagcgggctgcgggcccgaaacgggcccgccggagtcactcgctc

SEQ ID NO: 196 - DNA, 99 bp, Synthetic construct
  source 1..99: mol_type = other DNA; note = AAV2_Nb.BbvCI_BL
aaccggtgagggggagtcgcgcgagcgagcgagtgactccggcccgctggtttccagcgggctgcgggcccgaaacgggcccgccggagtcactcgctc

SEQ ID NO: 197 - DNA, 99 bp, Synthetic construct
  source 1..99: mol_type = other DNA; note = AAV2_Nb.BbvCI_TL
aaccggtgagggcgactcccgcgagcgagcgagtgactccggcccgctggtttccagcgggctgcgggcccgaaacgggcccgccggagtcactcgctc

SEQ ID NO: 198 - DNA, 99 bp, Synthetic construct
  source 1..99: mol_type = other DNA; note = AAV2_Nb.BsmI_BL
aaccggtgagggacttacgcgcgagcgagcgagtgactccggcccgctggtttccagcgggctgcgggcccgaaacgggcccgccggagtcactcgctc

SEQ ID NO: 199 - DNA, 99 bp, Synthetic construct
  source 1..99: mol_type = other DNA; note = AAV2_Nb.BsmI_TL
aaccggtgagggagagacgcgtaagcgagcgagtgactccggcccgctggtttccagcgggctgcgggcccgaaacgggcccgccggagtcactcgctc

SEQ ID NO: 200 - DNA, 99 bp, Synthetic construct
  source 1..99: mol_type = other DNA; note = AAV2_Nb.BsrDI_BL
aaccggtgagggcgttacgcgcgagcgagcgagtgactccggcccgctggtttccagcgggctgcgggcccgaaacgggcccgccggagtcactcgctc

SEQ ID NO: 201 - DNA, 99 bp, Synthetic construct
  source 1..99: mol_type = other DNA; note = AAV2_Nb.BsrDI_TL
aaccggtgagggagtaacgcgcgagcgagcgagtgactccggcccgctggtttccagcgggctgcgggcccgaaacgggcccgccggagtcactcgctc

SEQ ID NO: 202 - DNA, 99 bp, Synthetic construct
  source 1..99: mol_type = other DNA; note = AAV2_Nb.BssSI_BL
aaccggtgctcgagagacgcgcgagcgagcgagtgactccggcccgctggtttccagcgggctgcgggcccgaaacgggcccgccggagtcactcgctc

SEQ ID NO: 203 - DNA, 99 bp, Synthetic construct
  source 1..99: mol_type = other DNA; note = AAV2_Nb.BssSI_TL
aaccggtgagggagcaccgcgcgagcgagcgagtgactccggcccgctggtttccagcgggctgcgggcccgaaacgggcccgccggagtcactcgctc

SEQ ID NO: 204 - DNA, 99 bp, Synthetic construct
  source 1..99: mol_type = other DNA; note = AAV2_Nb.BtsI_BL
aaccggtgagggcgtcacgcgcgagcgagcgagtgactccggcccgctggtttccagcgggctgcgggcccgaaacgggcccgccggagtcactcgctc

SEQ ID NO: 205 - DNA, 99 bp, Synthetic construct
  source 1..99: mol_type = other DNA; note = AAV2_Nb.BtsI_TL
aaccggtgagggagtgacgcgcgagcgagcgagtgactccggcccgctggtttccagcgggctgcgggcccgaaacgggcccgccggagtcactcgctc

SEQ ID NO: 206 - DNA, 99 bp, Synthetic construct
  source 1..99: mol_type = other DNA; note = AAV2_Nt.AlwI_BL
aaccggtgagggagctaggcgcgagcgagcgagtgactccggcccgctggtttccagcgggctgcgggcccgaaacgggcccgccggagtcactcgctc

SEQ ID NO: 207 - DNA, 99 bp, Synthetic construct
  source 1..99: mol_type = other DNA; note = AAV2_Nt.AlwI_BL
aaccggtgacctagagacgcgcgagcgagcgagtgactccggcccgctggtttccagcgggctgcgggcccgaaacgggcccgccggagtcactcgctc

SEQ ID NO: 208 - DNA, 99 bp, Synthetic construct; identical to SEQ ID NO: 197
  source 1..99: mol_type = other DNA; note = AAV2_Nt.BbvCI_TL

SEQ ID NO: 209 - DNA, 99 bp, Synthetic construct; identical to SEQ ID NO: 196
  source 1..99: mol_type = other DNA; note = AAV2_Nt.BbvCI_BL

SEQ ID NO: 210 - DNA, 99 bp, Synthetic construct
  source 1..99: mol_type = other DNA; note = AAV2_Nt.BsmAI_TL
aaccggtgactctgagacgcgcgagcgagcgagtgactccggcccgctggtttccagcgggctgcgggcccgaaacgggcccgccggagtcactcgctc

SEQ ID NO: 211 - DNA, 99 bp, Synthetic construct
  source 1..99: mol_type = other DNA; note = AAV2_Nt.BsmAI_BL
aaccggtgaggcagagacgcgcgagcgagcgagtgactccggcccgctggtttccagcgggctgcgggcccgaaacgggcccgccggagtcactcgctc

SEQ ID NO: 212 - DNA, 99 bp, Synthetic construct
  source 1..99: mol_type = other DNA; note = AAV2_Nt.BspQI_TL
aaccggtgagggcttctcgcgcgagcgagcgagtgactccggcccgctggtttccagcgggctgcgggcccgaaacgggcccgccggagtcactcgctc

SEQ ID NO: 213 - DNA, 99 bp, Synthetic construct
  source 1..99: mol_type = other DNA; note = AAV2_Nt.BspQI_BL
aaccggtgagggcgagaagcgcgagcgagcgagtgactccggcccgctggtttccagcgggctgcgggcccgaaacgggcccgccggagtcactcgctc

SEQ ID NO: 214 - DNA, 100 bp, Synthetic construct
  source 1..100: mol_type = other DNA; note = AAV2_Nt.BstNBI_TL
aaccggtgagggagagacgcgctgagcgagcgagtgactccggcccgctggtttccagcgggctgcgggcccgaaacgggcccgccggagtcactcgctc

SEQ ID NO: 215 - DNA, 99 bp, Synthetic construct
  source 1..99: mol_type = other DNA; note = AAV2_Nt.BstNBI_BL
aaccgctcagggagagacgcgcgagcgagcgagtgactccggcccgctggtttccagcgggctgcgggcccgaaacgggcccgccggagtcactcgctc

SEQ ID NO: 216 - DNA, 99 bp, Synthetic construct
  source 1..99: mol_type = other DNA; note = wt_AAV3
aaccggtgagggagatacgcgtgagcgagcgagccaccccggaccgctggtttccagcggtctgcctgcacgaaacgtgcaggccggggtggctcgctc

SEQ ID NO: 217 - DNA, 99 bp, Synthetic construct
  source 1..99: mol_type = other DNA; note = AAV3_Nb.BbvCI_BL
aaccggtgagggggagtcgcgtgagcgagcgagccaccccggaccgctggtttccagcggtctgcctgcacgaaacgtgcaggccggggtggctcgctc

SEQ ID NO: 218 - DNA, 99 bp, Synthetic construct
  source 1..99: mol_type = other DNA; note = AAV3_Nb.BbvCI_TL
aaccggtgagggcgactcccgtgagcgagcgagccaccccggaccgctggtttccagcggtctgcctgcacgaaacgtgcaggccggggtggctcgctc

SEQ ID NO: 219 - DNA, 99 bp, Synthetic construct
  source 1..99: mol_type = other DNA; note = AAV3_Nb.BsmI_BL
aaccggtgagggacttacgcgtgagcgagcgagccaccccggaccgctggtttccagcggtctgcctgcacgaaacgtgcaggccggggtggctcgctc

SEQ ID NO: 220 - DNA, 99 bp, Synthetic construct
  source 1..99: mol_type = other DNA; note = AAV3_Nb.BsmI_TL
aaccggtgagggagatacgcgtaagcgagcgagccaccccggaccgctggtttccagcggtctgcctgcacgaaacgtgcaggccggggtggctcgctc

SEQ ID NO: 221 - DNA, 99 bp, Synthetic construct
  source 1..99: mol_type = other DNA; note = AAV3_Nb.BsrDI_BL
aaccggtgagggcgttacgcgtgagcgagcgagccaccccggaccgctggtttccagcggtctgcctgcacgaaacgtgcaggccggggtggctcgctc

SEQ ID NO: 222 - DNA, 99 bp, Synthetic construct
  source 1..99: mol_type = other DNA; note = AAV3_Nb.BsrDI_TL
aaccggtgagggagtaacgcgtgagcgagcgagccaccccggaccgctggtttccagcggtctgcctgcacgaaacgtgcaggccggggtggctcgctc

SEQ ID NO: 223 - DNA, 99 bp, Synthetic construct
  source 1..99: mol_type = other DNA; note = AAV3_Nb.BssSI_BL
aaccggtgctcgagatacgcgtgagcgagcgagccaccccggaccgctggtttccagcggtctgcctgcacgaaacgtgcaggccggggtggctcgctc

SEQ ID NO: 224 - DNA, 99 bp, Synthetic construct
  source 1..99: mol_type = other DNA; note = AAV3_Nb.BssSI_TL
aaccggtgagggagcaccgcgtgagcgagcgagccaccccggaccgctggtttccagcggtctgcctgcacgaaacgtgcaggccggggtggctcgctc

SEQ ID NO: 225 - DNA, 99 bp, Synthetic construct
  source 1..99: mol_type = other DNA; note = AAV3_Nb.BtsI_BL
aaccggtgagggcgtcacgcgtgagcgagcgagccaccccggaccgctggtttccagcggtctgcctgcacgaaacgtgcaggccggggtggctcgctc

SEQ ID NO: 226 - DNA, 99 bp, Synthetic construct
  source 1..99: mol_type = other DNA; note = AAV3_Nb.BtsI_TL
aaccggtgagggagtgacgcgtgagcgagcgagccaccccggaccgctggtttccagcggtctgcctgcacgaaacgtgcaggccggggtggctcgctc

SEQ ID NO: 227 - DNA, 99 bp, Synthetic construct
  source 1..99: mol_type = other DNA; note = AAV3_Nt.AlwI_BL
aaccggtgagggagctaggcgtgagcgagcgagccaccccggaccgctggtttccagcggtctgcctgcacgaaacgtgcaggccggggtggctcgctc

SEQ ID NO: 228 - DNA, 99 bp, Synthetic construct
  source 1..99: mol_type = other DNA; note = AAV3_Nt.AlwI_BL
aaccggtgacctagatacgcgtgagcgagcgagccaccccggaccgctggtttccagcggtctgcctgcacgaaacgtgcaggccggggtggctcgctc

SEQ ID NO: 229 - DNA, 99 bp, Synthetic construct; identical to SEQ ID NO: 218
  source 1..99: mol_type = other DNA; note = AAV3_Nt.BbvCI_TL

SEQ ID NO: 230 - DNA, 99 bp, Synthetic construct; identical to SEQ ID NO: 217
  source 1..99: mol_type = other DNA; note = AAV3_Nt.BbvCI_BL

SEQ ID NO: 231 - DNA, 99 bp, Synthetic construct
  source 1..99: mol_type = other DNA; note = AAV3_Nt.BsmAI_TL
aaccggtgactctgatacgcgtgagcgagcgagccaccccggaccgctggtttccagcggtctgcctgcacgaaacgtgcaggccggggtggctcgctc

SEQ ID NO: 232 - DNA, 99 bp, Synthetic construct
  source 1..99: mol_type = other DNA; note = AAV3_Nt.BsmAI_BL
aaccggtgaggcagagacgcgtgagcgagcgagccaccccggaccgctggtttccagcggtctgcctgcacgaaacgtgcaggccggggtggctcgctc

SEQ ID NO: 233 - DNA, 99 bp, Synthetic construct
  source 1..99: mol_type = other DNA; note = AAV3_Nt.BspQI_TL
aaccggtgagggcttctcgcgtgagcgagcgagccaccccggaccgctggtttccagcggtctgcctgcacgaaacgtgcaggccggggtggctcgctc

SEQ ID NO: 234 - DNA, 99 bp, Synthetic construct
  source 1..99: mol_type = other DNA; note = AAV3_Nt.BspQI_BL
aaccggtgagggcgagaagcgtgagcgagcgagccaccccggaccgctggtttccagcggtctgcctgcacgaaacgtgcaggccggggtggctcgctc

SEQ ID NO: 235 - DNA, 100 bp, Synthetic construct
  source 1..100: mol_type = other DNA; note = AAV3_Nt.BstNBI_TL
aaccggtgagggagatacgcgctgagcgagcgagccaccccggaccgctggtttccagcggtctgcctgcacgaaacgtgcaggccggggtggctcgctc

SEQ ID NO: 236 - DNA, 99 bp, Synthetic construct
  source 1..99: mol_type = other DNA; note = AAV3_Nt.BstNBI_BL
aaccgctcagggagatacgcgtgagcgagcgagccaccccggaccgctggtttccagcggtctgcctgcacgaaacgtgcaggccggggtggctcgctc

SEQ ID NO: 237 - DNA, 125 bp, Synthetic construct
  source 1..125: mol_type = other DNA; note = wt_AAV4_left
aaccggtgagggagatacgcgcgagcgagtgagtgagccgggacctctggtttccagaggtctgacggccggagaccggccgtcccggctcactcactcgctcgcgcgtatctccctcaccggtt

SEQ ID NO: 238 - DNA, 125 bp, Synthetic construct
  source 1..125: mol_type = other DNA; note = AAV4_left_Nb.BbvCI_BL
aaccggtgagggggagtcgcgcgagcgagtgagtgagccgggacctctggtttccagaggtctgacggccggagaccggccgtcccggctcactcactcgctcgcgcgactccccctcaccggtt

SEQ ID NO: 239 - DNA, 125 bp, Synthetic construct
  source 1..125: mol_type = other DNA; note = AAV4_left_Nb.BbvCI_TL
aaccggtgagggcgactcccgcgagcgagtgagtgagccgggacctctggtttccagaggtctgacggccggagaccggccgtcccggctcactcactcgctcgcgggagtcgccctcaccggtt

SEQ ID NO: 240 - DNA, 125 bp, Synthetic construct
  source 1..125: mol_type = other DNA; note = AAV4_left_Nb.BsmI_BL
aaccggtgagggacttacgcgcgagcgagtgagtgagccgggacctctggtttccagaggtctgacggccggagaccggccgtcccggctcactcactcgctcgcgcgtaagtccctcaccggtt

SEQ ID NO: 241 - DNA, 125 bp, Synthetic construct
  source 1..125: mol_type = other DNA; note = AAV4_left_Nb.BsmI_TL
aaccggtgagggagatacgcgtaagcgagtgagtgagccgggacctctggtttccagaggtctgacggccggagaccggccgtcccggctcactcactcgcttacgcgtatctccctcaccggtt

SEQ ID NO: 242 - DNA, 125 bp, Synthetic construct
  source 1..125: mol_type = other DNA; note = AAV4_left_Nb.BsrDI_BL
aaccggtgagggcgttacgcgcgagcgagtgagtgagccgggacctctggtttccagaggtctgacggccggagaccggccgtcccggctcactcactcgctcgcgcgtaacgccctcaccggtt

SEQ ID NO: 243 - DNA, 125 bp, Synthetic construct
  source 1..125: mol_type = other DNA; note = AAV4_left_Nb.BsrDI_TL
aaccggtgagggagtaacgcgcgagcgagtgagtgagccgggacctctggtttccagaggtctgacggccggagaccggccgtcccggctcactcactcgctcgcgcgttactccctcaccggtt

SEQ ID NO: 244 - DNA, 125 bp, Synthetic construct
  source 1..125: mol_type = other DNA; note = AAV4_left_Nb.BssSI_BL
aaccggtgctcgagatacgcgcgagcgagtgagtgagccgggacctctggtttccagaggtctgacggccggagaccggccgtcccggctcactcactcgctcgcgcgtatctcgagcaccggtt

SEQ ID NO: 245 - DNA, 125 bp, Synthetic construct
  source 1..125: mol_type = other DNA; note = AAV4_left_Nb.BssSI_TL
aaccggtgagggagcaccgcgcgagcgagtgagtgagccgggacctctggtttccagaggtctgacggccggagaccggccgtcccggctcactcactcgctcgcgcggtgctccctcaccggtt

SEQ ID NO: 246 - DNA, 125 bp, Synthetic construct
  source 1..125: mol_type = other DNA; note = AAV4_left_Nb.BtsI_BL
aaccggtgagggcgtcacgcgcgagcgagtgagtgagccgggacctctggtttccagaggtctgacggccggagaccggccgtcccggctcactcactcgctcgcgcgtgacgccctcaccggtt

SEQ ID NO: 247 - DNA, 125 bp, Synthetic construct
  source 1..125: mol_type = other DNA; note = AAV4_left_Nb.BtsI_TL
aaccggtgagggagtgacgcgcgagcgagtgagtgagccgggacctctggtttccagaggtctgacggccggagaccggccgtcccggctcactcactcgctcgcgcgtcactccctcaccggtt

SEQ ID NO: 248 - DNA, 125 bp, Synthetic construct
  source 1..125: mol_type = other DNA; note = AAV4_left_Nt.AlwI_BL
aaccggtgagggagctaggcgcgagcgagtgagtgagccgggacctctggtttccagaggtctgacggccggagaccggccgtcccggctcactcactcgctcgcgcctagctccctcaccggtt

SEQ ID NO: 249 - DNA, 125 bp, Synthetic construct
  source 1..125: mol_type = other DNA; note = AAV4_left_Nt.AlwI_BL
aaccggtgacctagatacgcgcgagcgagtgagtgagccgggacctctggtttccagaggtctgacggccggagaccggccgtcccggctcactcactcgctcgcgcgtatctaggtcaccggtt

SEQ ID NO: 250 - DNA, 125 bp, Synthetic construct; identical to SEQ ID NO: 239
  source 1..125: mol_type = other DNA; note = AAV4_left_Nt.BbvCI_TL

SEQ ID NO: 251 - DNA, 125 bp, Synthetic construct; identical to SEQ ID NO: 238
  source 1..125: mol_type = other DNA; note = AAV4_left_Nt.BbvCI_BL

SEQ ID NO: 252 - DNA, 125 bp, Synthetic construct
  source 1..125: mol_type = other DNA; note = AAV4_left_Nt.BsmAI_TL
aaccggtgactctgatacgcgcgagcgagtgagtgagccgggacctctggtttccagaggtctgacggccggagaccggccgtcccggctcactcactcgctcgcgcgtatcagagtcaccggtt

SEQ ID NO: 253 - DNA, 125 bp, Synthetic construct
  source 1..125: mol_type = other DNA; note = AAV4_left_Nt.BsmAI_BL
aaccggtgaggcagagacgcgcgagcgagtgagtgagccgggacctctggtttccagaggtctgacggccggagaccggccgtcccggctcactcactcgctcgcgcgtctctgcctcaccggtt

SEQ ID NO: 254 - DNA, 125 bp, Synthetic construct
  source 1..125: mol_type = other DNA; note = AAV4_left_Nt.BspQI_TL
aaccggtgagggcttctcgcgcgagcgagtgagtgagccgggacctctggtttccagaggtctgacggccggagaccggccgtcccggctcactcactcgctcgcgcgagaagccctcaccggtt

SEQ ID NO: 255 - DNA, 125 bp, Synthetic construct
  source 1..125: mol_type = other DNA; note = AAV4_left_Nt.BspQI_BL
aaccggtgagggcgagaagcgcgagcgagtgagtgagccgggacctctggtttccagaggtctgacggccggagaccggccgtcccggctcactcactcgctcgcgcttctcgccctcaccggtt

SEQ ID NO: 256 - DNA, 127 bp, Synthetic construct
  source 1..127: mol_type = other DNA; note = AAV4_left_Nt.BstNBI_TL
aaccggtgagggagatacgcgctgagcgagtgagtgagccgggacctctggtttccagaggtctgacggccggagaccggccgtcccggctcactcactcgctcagcgcgtatctccctcaccggtt

SEQ ID NO: 257 - DNA, 125 bp, Synthetic construct
  source 1..125: mol_type = other DNA; note = AAV4_left_Nt.BstNBI_BL
aaccgctcagggagatacgcgcgagcgagtgagtgagccgggacctctggtttccagaggtctgacggccggagaccggccgtcccggctcactcactcgctcgcgcgtatctccctgagcggtt

SEQ ID NO: 258 - DNA, 125 bp, Synthetic construct
  source 1..125: mol_type = other DNA; note = wt_AAV4_Right
aaccggtgtaatcgatacgcgcgagcgagtgagtgagccgggacctctggtttccagaggtctgacggccggagaccggccgtcccggctcactcactcgctcgcgcgtatctccctcaccggtt

SEQ ID NO: 259 - DNA, 125 bp, Synthetic construct
  source 1..125: mol_type = other DNA; note = AAV4_Right_Nb.BbvCI_BL
aaccggtgtaatggagtcgcgcgagcgagtgagtgagccgggacctctggtttccagaggtctgacggccggagaccggccgtcccggctcactcactcgctcgcgcgactccccctcaccggtt

SEQ ID NO: 260 - DNA, 125 bp, Synthetic construct
  source 1..125: mol_type = other DNA; note = AAV4_Right_Nb.BbvCI_TL
aaccggtgtaatcgactcccgcgagcgagtgagtgagccgggacctctggtttccagaggtctgacggccggagaccggccgtcccggctcactcactcgctcgcgggagtcgccctcaccggtt

SEQ ID NO: 261 - DNA, 125 bp, Synthetic construct
  source 1..125: mol_type = other DNA; note = AAV4_Right_Nb.BsmI_BL
aaccggtgtaatccttacgcgcgagcgagtgagtgagccgggacctctggtttccagaggtctgacggccggagaccggccgtcccggctcactcactcgctcgcgcgtaagtccctcaccggtt

SEQ ID NO: 262 - DNA, 125 bp, Synthetic construct
  source 1..125: mol_type = other DNA; note = AAV4_Right_Nb.BsmI_TL
aaccggtgtaatcgatacgcgtaagcgagtgagtgagccgggacctctggtttccagaggtctgacggccggagaccggccgtcccggctcactcactcgcttacgcgtatctccctcaccggtt

SEQ ID NO: 263 - DNA, 125 bp, Synthetic construct
  source 1..125: mol_type = other DNA; note = AAV4_Right_Nb.BsrDI_BL
aaccggtgtaatcgttacgcgcgagcgagtgagtgagccgggacctctggtttccagaggtctgacggccggagaccggccgtcccggctcactcactcgctcgcgcgtaacgccctcaccggtt

SEQ ID NO: 264 - DNA, 125 bp, Synthetic construct
  source 1..125: mol_type = other DNA; note = AAV4_Right_Nb.BsrDI_TL
aaccggtgtaatcgtaacgcgcgagcgagtgagtgagccgggacctctggtttccagaggtctgacggccggagaccggccgtcccggctcactcactcgctcgcgcgttactccctcaccggtt

SEQ ID NO: 265 - DNA, 125 bp, Synthetic construct
  source 1..125: mol_type = other DNA; note = AAV4_Right_Nb.BssSI_BL
aaccggtgctctcgatacgcgcgagcgagtgagtgagccgggacctctggtttccagaggtctgacggccggagaccggccgtcccggctcactcactcgctcgcgcgtatctcgagcaccggtt

SEQ ID NO: 266 - DNA, 125 bp, Synthetic construct
  source 1..125: mol_type = other DNA; note = AAV4_Right_Nb.BssSI_TL
aaccggtgtaagagcaccgcgcgagcgagtgagtgagccgggacctctggtttccagaggtctgacggccggagaccggccgtcccggctcactcactcgctcgcgcggtgctccctcaccggtt

SEQ ID NO: 267 - DNA, 125 bp, Synthetic construct
  source 1..125: mol_type = other DNA; note = AAV4_Right_Nb.BtsI_BL
aaccggtgtaatcgtcacgcgcgagcgagtgagtgagccgggacctctggtttccagaggtctgacggccggagaccggccgtcccggctcactcactcgctcgcgcgtgacgccctcaccggtt

SEQ ID NO: 268 - DNA, 125 bp, Synthetic construct
  source 1..125: mol_type = other DNA; note = AAV4_Right_Nb.BtsI_TL
aaccggtgtaatcgtgacgcgcgagcgagtgagtgagccgggacctctggtttccagaggtctgacggccggagaccggccgtcccggctcactcactcgctcgcgcgtcactccctcaccggtt

SEQ ID NO: 269 - DNA, 125 bp, Synthetic construct
  source 1..125: mol_type = other DNA; note = AAV4_Right_Nt.AlwI_BL
aaccggtgtaatcgctaggcgcgagcgagtgagtgagccgggacctctggtttccagaggtctgacggccggagaccggccgtcccggctcactcactcgctcgcgcctagctccctcaccggtt

SEQ ID NO: 270 - DNA, 125 bp, Synthetic construct
  source 1..125: mol_type = other DNA; note = AAV4_Right_Nt.AlwI_BL
aaccggtgtcctagatacgcgcgagcgagtgagtgagccgggacctctggtttccagaggtctgacggccggagaccggccgtcccggctcactcactcgctcgcgcgtatctaggtcaccggtt

SEQ ID NO: 271 - DNA, 125 bp, Synthetic construct; identical to SEQ ID NO: 260
  source 1..125: mol_type = other DNA; note = AAV4_Right_Nt.BbvCI_TL

SEQ ID NO: 272 - DNA, 125 bp, Synthetic construct; identical to SEQ ID NO: 259
  source 1..125: mol_type = other DNA; note = AAV4_Right_Nt.BbvCI_BL

SEQ ID NO: 273 - DNA, 125 bp, Synthetic construct
  source 1..125: mol_type = other DNA; note = AAV4_Right_Nt.BsmAI_TL
aaccggtgtctctgatacgcgcgagcgagtgagtgagccgggacctctggtttccagaggtctgacggccggagaccggccgtcccggctcactcactcgctcgcgcgtatcagagtcaccggtt

SEQ ID NO: 274 - DNA, 125 bp, Synthetic construct
  source 1..125: mol_type = other DNA; note = AAV4_Right_Nt.BsmAI_BL
aaccggtgtaacagagacgcgcgagcgagtgagtgagccgggacctctggtttccagaggtctgacggccggagaccggccgtcccggctcactcactcgctcgcgcgtctctgcctcaccggtt

SEQ ID NO: 275 - DNA, 125 bp, Synthetic construct
  source 1..125: mol_type = other DNA; note = AAV4_Right_Nt.BspQI_TL
aaccggtgtaatcttctcgcgcgagcgagtgagtgagccgggacctctggtttccagaggtctgacggccggagaccggccgtcccggctcactcactcgctcgcgcgagaagccctcaccggtt

SEQ ID NO: 276 - DNA, 125 bp, Synthetic construct
  source 1..125: mol_type = other DNA; note = AAV4_Right_Nt.BspQI_BL
aaccggtgtaatcgagaagcgcgagcgagtgagtgagccgggacctctggtttccagaggtctgacggccggagaccggccgtcccggctcactcactcgctcgcgcttctcgccctcaccggtt

SEQ ID NO: 277 - DNA, 127 bp, Synthetic construct
  source 1..127: mol_type = other DNA; note = AAV4_Right_Nt.BstNBI_TL
aaccggtgtaatcgatacgcgctgagcgagtgagtgagccgggacctctggtttccagaggtctgacggccggagaccggccgtcccggctcactcactcgctcagcgcgtatctccctcaccggtt

SEQ ID NO: 278 - DNA, 125 bp, Synthetic construct
  source 1..125: mol_type = other DNA; note = AAV4_Right_Nt.BstNBI_BL
aaccggtctcagcgatacgcgcgagcgagtgagtgagccgggacctctggtttccagaggtctgacggccggagaccggccgtcccggctcactcactcgctcgcgcgtatctctgagaccggtt

SEQ ID NO: 279 - DNA, 137 bp, Synthetic construct
  source 1..137: mol_type = other DNA; note = wt_AAV5
gagaggggggacagcgcaagcgagcgagcgaccgagcaaacccccccaccgtcgagtttctcgacggtctgctgccgggagaccggcagcgggggggtttgctcggtcgctcgctcgcttgcgctgtcccccctctc

SEQ ID NO: 280 - DNA, 137 bp, Synthetic construct
  source 1..137: mol_type = other DNA; note = AAV5_Nb.BbvCI_BL
gagaggggagtcggcgcaagcgagcgagcgaccgagcaaacccccccaccgtcgagtttctcgacggtctgctgccgggagaccggcagcgggggggtttgctcggtcgctcgctcgcttgcgccgactcccctctc

SEQ ID NO: 281 - DNA, 137 bp, Synthetic construct
  source 1..137: mol_type = other DNA; note = AAV5_Nb.BbvCI_TL
gagaggggcgactccgcaagcgagcgagcgaccgagcaaacccccccaccgtcgagtttctcgacggtctgctgccgggagaccggcagcgggggggtttgctcggtcgctcgctcgcttgcggagtcgcccctctc

SEQ ID NO: 282 - DNA, 137 bp, Synthetic construct
  source 1..137: mol_type = other DNA; note = AAV5_Nb.BsmI_BL
gagaggggcttacgcgcaagcgagcgagcgaccgagcaaacccccccaccgtcgagtttctcgacggtctgctgccgggagaccggcagcgggggggtttgctcggtcgctcgctcgcttgcgcgtaagcccctctc

SEQ ID NO: 283 - DNA, 137 bp, Synthetic construct
  source 1..137: mol_type = other DNA; note = AAV5_Nb.BsmI_TL
gagaggggggacagcgtaagcgagcgagcgaccgagcaaacccccccaccgtcgagtttctcgacggtctgctgccgggagaccggcagcgggggggtttgctcggtcgctcgctcgcttacgctgtcccccctctc

SEQ ID NO: 284 - DNA, 137 bp, Synthetic construct
  source 1..137: mol_type = other DNA; note = AAV5_Nb.BsrDI_BL
gagaggcgttacagcgcaagcgagcgagcgaccgagcaaacccccccaccgtcgagtttctcgacggtctgctgccgggagaccggcagcgggggggtttgctcggtcgctcgctcgcttgcgctgtaacgcctctc

SEQ ID NO: 285 - DNA, 137 bp, Synthetic construct
  source 1..137: mol_type = other DNA; note = AAV5_Nb.BsrDI_TL
gagagggggtaacgcgcaagcgagcgagcgaccgagcaaacccccccaccgtcgagtttctcgacggtctgctgccgggagaccggcagcgggggggtttgctcggtcgctcgctcgcttgcgcgttaccccctctc

SEQ ID NO: 286 - DNA, 137 bp, Synthetic construct
  source 1..137: mol_type = other DNA; note = AAV5_Nb.BssSI_BL
gagtgctcggacagcgcaagcgagcgagcgaccgagcaaacccccccaccgtcgagtttctcgacggtctgctgccgggagaccggcagcgggggggtttgctcggtcgctcgctcgcttgcgctgtccgagcactc

SEQ ID NO: 287 - DNA, 137 bp, Synthetic construct
  source 1..137: mol_type = other DNA; note = AAV5_Nb.BssSI_TL
gagagggagcacagcgcaagcgagcgagcgaccgagcaaacccccccaccgtcgagtttctcgacggtctgctgccgggagaccggcagcgggggggtttgctcggtcgctcgctcgcttgcgctgtgctccctctc

SEQ ID NO: 288 - DNA, 137 bp, Synthetic construct
  source 1..137: mol_type = other DNA; note = AAV5_Nb.BtsI_BL
gagaggcgtcacagcgcaagcgagcgagcgaccgagcaaacccccccaccgtcgagtttctcgacggtctgctgccgggagaccggcagcgggggggtttgctcggtcgctcgctcgcttgcgctgtgacgcctctc

SEQ ID NO: 289 - DNA, 137 bp, Synthetic construct
  source 1..137: mol_type = other DNA; note = AAV5_Nb.BtsI_TL
gagaggggtgacggcgcaagcgagcgagcgaccgagcaaacccccccaccgtcgagtttctcgacggtctgctgccgggagaccggcagcgggggggtttgctcggtcgctcgctcgcttgcgccgtcacccctctc

SEQ ID NO: 290 - DNA, 137 bp, Synthetic construct
  source 1..137: mol_type = other DNA; note = AAV5_Nt.AlwI_BL
gagaggggggacagcgctagggagcgagcgaccgagcaaacccccccaccgtcgagtttctcgacggtctgctgccgggagaccggcagcgggggggtttgctcggtcgctcgctccctagcgctgtcccccctctc

SEQ ID NO: 291 - DNA, 137 bp, Synthetic construct
  source 1..137: mol_type = other DNA; note = AAV5_Nt.AlwI_BL
gagagggggcctagcgcaagcgagcgagcgaccgagcaaacccccccaccgtcgagtttctcgacggtctgctgccgggagaccggcagcgggggggtttgctcggtcgctcgctcgcttgcgctaggccccctctc

SEQ ID NO: 292 - DNA, 137 bp, Synthetic construct; identical to SEQ ID NO: 281
  source 1..137: mol_type = other DNA; note = AAV5_Nt.BbvCI_TL

SEQ ID NO: 293 - DNA, 137 bp, Synthetic construct; identical to SEQ ID NO: 280
  source 1..137: mol_type = other DNA; note = AAV5_Nt.BbvCI_BL

SEQ ID NO: 294 - DNA, 137 bp, Synthetic construct
  source 1..137: mol_type = other DNA; note = AAV5_Nt.BsmAI_TL
gagagggggctctgcgcaagcgagcgagcgaccgagcaaacccccccaccgtcgagtttctcgacggtctgctgccgggagaccggcagcgggggggtttgctcggtcgctcgctcgcttgcgcagagccccctctc

SEQ ID NO: 295 - DNA, 137 bp, Synthetic construct
  source 1..137: mol_type = other DNA; note = AAV5_Nt.BsmAI_BL
gagagggggcagagcgcaagcgagcgagcgaccgagcaaacccccccaccgtcgagtttctcgacggtctgctgccgggagaccggcagcgggggggtttgctcggtcgctcgctcgcttgcgctctgccccctctc

SEQ ID NO: 296 - DNA, 137 bp, Synthetic construct
  source 1..137: mol_type = other DNA; note = AAV5_Nt.BspQI_TL
gagagggcttctcgcgcaagcgagcgagcgaccgagcaaacccccccaccgtcgagtttctcgacggtctgctgccgggagaccggcagcgggggggtttgctcggtcgctcgctcgcttgcgcgagaagccctctc

SEQ ID NO: 297 - DNA, 137 bp, Synthetic construct
  source 1..137: mol_type = other DNA; note = AAV5_Nt.BspQI_BL
gagagggcgagaagcgcaagcgagcgagcgaccgagcaaacccccccaccgtcgagtttctcgacggtctgctgccgggagaccggcagcgggggggtttgctcggtcgctcgctcgcttgcgcttctcgccctctc

SEQ ID NO: 298 - DNA, 139 bp, Synthetic construct
  source 1..139: mol_type = other DNA; note = AAV5_Nt.BstNBI_TL
gagaggggggacagcgctgagcgagcgagcgaccgagcaaacccccccaccgtcgagtttctcgacggtctgctgccgggagaccggcagcgggggggtttgctcggtcgctcgctcgctcagcgctgtcccccctctc

SEQ ID NO: 299 - DNA, 137 bp, Synthetic construct
  source 1..137: mol_type = other DNA; note = AAV5_Nt.BstNBI_BL
gagagggggctcagcgcaagcgagcgagcgaccgagcaaacccccccaccgtcgagtttctcgacggtctgctgccgggagaccggcagcgggggggtttgctcggtcgctcgctcgcttgcgctgagccccctctc

SEQ ID NO: 300 - DNA, 125 bp, Synthetic construct
  source 1..125: mol_type = other DNA; note = wt_AAV7
aaccggtgagggagatacgcgcgagcgagcgagccaccccggacgcctggtttccaggcgtctgccgtctcgagacgagacggccggggtggctcgctcgctcgcgcgtatctccctcaccggtt

SEQ ID NO: 301 - DNA, 125 bp, Synthetic construct
  source 1..125: mol_type = other DNA; note = AAV7_Nb.BbvCI_BL
aaccggtgagggggagtcgcgcgagcgagcgagccaccccggacgcctggtttccaggcgtctgccgtctcgagacgagacggccggggtggctcgctcgctcgcgcgactccccctcaccggtt

SEQ ID NO: 302 - DNA, 125 bp, Synthetic construct
  source 1..125: mol_type = other DNA; note = AAV7_Nb.BbvCI_TL
aaccggtgagggcgactcccgcgagcgagcgagccaccccggacgcctggtttccaggcgtctgccgtctcgagacgagacggccggggtggctcgctcgctcgcgggagtcgccctcaccggtt

SEQ ID NO: 303 - DNA, 125 bp, Synthetic construct
  source 1..125: mol_type = other DNA; note = AAV7_Nb.BsmI_BL
aaccggtgagggacttacgcgcgagcgagcgagccaccccggacgcctggtttccaggcgtctgccgtctcgagacgagacggccggggtggctcgctcgctcgcgcgtaagtccctcaccggtt

SEQ ID NO: 304 - DNA, 125 bp, Synthetic construct
  source 1..125: mol_type = other DNA; note = AAV7_Nb.BsmI_TL
aaccggtgagggagatacgcgtaagcgagcgagccaccccggacgcctggtttccaggcgtctgccgtctcgagacgagacggccggggtggctcgctcgcttacgcgtatctccctcaccggtt

SEQ ID NO: 305 - DNA, 125 bp, Synthetic construct
  source 1..125: mol_type = other DNA; note = AAV7_Nb.BsrDI_BL
aaccggtgagggcgttacgcgcgagcgagcgagccaccccggacgcctggtttccaggcgtctgccgtctcgagacgagacggccggggtggctcgctcgctcgcgcgtaacgccctcaccggtt

SEQ ID NO: 306 - DNA, 125 bp, Synthetic construct
  source 1..125: mol_type = other DNA; note = AAV7_Nb.BsrDI_TL
aaccggtgagggagtaacgcgcgagcgagcgagccaccccggacgcctggtttccaggcgtctgccgtctcgagacgagacggccggggtggctcgctcgctcgcgcgttactccctcaccggtt

SEQ ID NO: 307 - DNA, 125 bp, Synthetic construct
  source 1..125: mol_type = other DNA; note = AAV7_Nb.BssSI_BL
aaccggtgctcgagatacgcgcgagcgagcgagccaccccggacgcctggtttccaggcgtctgccgtctcgagacgagacggccggggtggctcgctcgctcgcgcgtatctcgagcaccggtt

SEQ ID NO: 308 - DNA, 125 bp, Synthetic construct
  source 1..125: mol_type = other DNA; note = AAV7_Nb.BssSI_TL
aaccggtgagggagcaccgcgcgagcgagcgagccaccccggacgcctggtttccaggcgtctgccgtctcgagacgagacggccggggtggctcgctcgctcgcgcggtgctccctcaccggtt

SEQ ID NO: 309 - DNA, 125 bp, Synthetic construct
  source 1..125: mol_type = other DNA; note = AAV7_Nb.BtsI_BL
aaccggtgagggcgtcacgcgcgagcgagcgagccaccccggacgcctggtttccaggcgtctgccgtctcgagacgagacggccggggtggctcgctcgctcgcgcgtgacgccctcaccggtt

SEQ ID NO: 310 - DNA, 125 bp, Synthetic construct
  source 1..125: mol_type = other DNA; note = AAV7_Nb.BtsI_TL
aaccggtgagggagtgacgcgcgagcgagcgagccaccccggacgcctggtttccaggcgtctgccgtctcgagacgagacggccggggtggctcgctcgctcgcgcgtcactccctcaccggtt

SEQ ID NO: 311 - DNA, 125 bp, Synthetic construct
  source 1..125: mol_type = other DNA; note = AAV7_Nt.AlwI_BL
aaccggtgagggagctaggcgcgagcgagcgagccaccccggacgcctggtttccaggcgtctgccgtctcgagacgagacggccggggtggctcgctcgctcgcgcctagctccctcaccggtt

SEQ ID NO: 312 - DNA, 125 bp, Synthetic construct
  source 1..125: mol_type = other DNA; note = AAV7_Nt.AlwI_BL
aaccggtgacctagatacgcgcgagcgagcgagccaccccggacgcctggtttccaggcgtctgccgtctcgagacgagacggccggggtggctcgctcgctcgcgcgtatctaggtcaccggtt

SEQ ID NO: 313 - DNA, 125 bp, Synthetic construct; identical to SEQ ID NO: 302
  source 1..125: mol_type = other DNA; note = AAV7_Nt.BbvCI_TL

SEQ ID NO: 314 - DNA, 125 bp, Synthetic construct; identical to SEQ ID NO: 301
  source 1..125: mol_type = other DNA; note = AAV7_Nt.BbvCI_BL

SEQ ID NO: 315 - DNA, 125 bp, Synthetic construct
  source 1..125: mol_type = other DNA; note = AAV7_Nt.BsmAI_TL
aaccggtgactctgatacgcgcgagcgagcgagccaccccggacgcctggtttccaggcgtctgccgtctcgagacgagacggccggggtggctcgctcgctcgcgcgtatcagagtcaccggtt

SEQ ID NO: 316 - DNA, 125 bp, Synthetic construct
  source 1..125: mol_type = other DNA; note = AAV7_Nt.BsmAI_BL
aaccggtgaggcagagacgcgcgagcgagcgagccaccccggacgcctggtttccaggcgtctgccgtctcgagacgagacggccggggtggctcgctcgctcgcgcgtctctgcctcaccggtt

SEQ ID NO: 317 - DNA, 125 bp, Synthetic construct
  source 1..125: mol_type = other DNA; note = AAV7_Nt.BspQI_TL
aaccggtgagggcttctcgcgcgagcgagcgagccaccccggacgcctggtttccaggcgtctgccgtctcgagacgagacggccggggtggctcgctcgctcgcgcgagaagccctcaccggtt

SEQ ID NO: 318 - DNA, 125 bp, Synthetic construct
  source 1..125: mol_type = other DNA; note = AAV7_Nt.BspQI_BL
aaccggtgagggcgagaagcgcgagcgagcgagccaccccggacgcctggtttccaggcgtctgccgtctcgagacgagacggccggggtggctcgctcgctcgcgcttctcgccctcaccggtt

SEQ ID NO: 319 - DNA, 127 bp, Synthetic construct
  source 1..127: mol_type = other DNA; note = AAV7_Nt.BstNBI_TL
aaccggtgagggagatacgcgctgagcgagcgagccaccccggacgcctggtttccaggcgtctgccgtctcgagacgagacggccggggtggctcgctcgctcagcgcgtatctccctcaccggtt

SEQ ID NO: 320 - DNA, 125 bp, Synthetic construct
  source 1..125: mol_type = other DNA; note = AAV7_Nt.BstNBI_BL
aaccgctcagggagatacgcgcgagcgagcgagccaccccggacgcctggtttccaggcgtctgccgtctcgagacgagacggccggggtggctcgctcgctcgcgcgtatctccctgagcggtt

SEQ ID NO: 321-324 - skipped (4 entries)

SEQ ID NO: 325 - DNA, 125 bp, Synthetic construct
  source 1..125: mol_type = other DNA
ttggtcactccctctctgtacactcactcactcactgatccctggataccaaaggtatccagacacccagtctttgactgggtgggatcagtgagtgagtgagtgtacagagagggagtgaccaa

SEQ ID NO: 326-375 - skipped (50 entries)

SEQ ID NO: 376 - DNA, 27 bp, Synthetic construct
  source 1..27: mol_type = other DNA; note = I-CeuI
taactataacggtcctaaggtagcgaa

SEQ ID NO: 377 - DNA, 18 bp, Synthetic construct
  source 1..18: mol_type = other DNA; note = I-SceI
tagggataacagggtaat

SEQ ID NO: 378 - DNA, 15 bp, Synthetic construct
  source 1..15: mol_type = other DNA; note = I-PpoI
ctctcttaaggtagc

SEQ ID NO: 379-471 - skipped (93 entries)

SEQ ID NO: 472 - DNA, 12 bp, Adeno-associated virus
  source 1..12: mol_type = genomic DNA; note = Corresponding RABS Sequences Rep (AAV1,2,7)
gctcgctcgctc

SEQ ID NO: 473 - DNA, 12 bp, Adeno-associated virus
  source 1..12: mol_type = genomic DNA; note = Reverse Complement RABSRep (AAV1,2,7)
gagcgagcgagc

SEQ ID NO: 474 - DNA, 12 bp, Adeno-associated virus
  source 1..12: mol_type = genomic DNA; note = Corresponding RABS Sequences Rep (AAV3)
gatcgctcgctc

SEQ ID NO: 475 - DNA, 12 bp, Adeno-associated virus
  source 1..12: mol_type = genomic DNA; note = Reverse Complement RABS Rep (AAV3)
gagcgagcgatc

SEQ ID NO: 476 - DNA, 16 bp, Adeno-associated virus
  source 1..16: mol_type = genomic DNA; note = Corresponding RABS Sequences Rep (AAV4)
gctcgctcactcactc

SEQ ID NO: 477 - DNA, 16 bp, Adeno-associated virus
  source 1..16: mol_type = genomic DNA; note = Reverse Complement RABS Rep (AAV4)
gagtgagtgagcgagc

SEQ ID NO: 478 - DNA, 16 bp, Adeno-associated virus
  source 1..16: mol_type = genomic DNA; note = Corresponding RABS Sequences Rep (AAV5)
gctcgctcgctggctc

SEQ ID NO: 479 - DNA, 16 bp, Adeno-associated virus
  source 1..16: mol_type = genomic DNA; note = Reverse Complement RABS Rep (AAV5)
gagccagcgagcgagc

SEQ ID NO: 480-483 - skipped (4 entries)

SEQ ID NO: 484 - DNA, 10 bp, Adeno-associated virus
  source 1..10: mol_type = genomic DNA; note = Corresponding RABS Sequences NS1-NSBE3 (B19V)
ttccggtaca

SEQ ID NO: 485 - DNA, 10 bp, Adeno-associated virus
  source 1..10: mol_type = genomic DNA; note = Reverse Complement RABS NS1-NSBE3 (B19V)
tgtaccggaa

SEQ ID NO: 486 - DNA, 121 bp, Adeno-associated virus
  source 1..121: mol_type = genomic DNA; note = RAPS recognition sequence
ggccactcccgaagagcgcgctcgctatctcactgaggccgggcgaccaaaggtcgcccgacgcccgggctttgcccgggcggcctcagtgagatagcgagcgcgctcttcgggagtggcc

SEQ ID NO: 487-492 - skipped (6 entries)

SEQ ID NO: 493 - DNA, 23 bp, Synthetic construct
  source 1..23: mol_type = other DNA; note = AAV2 wt gRNA for Nicking Cas9
agcgagcgagcgcgcagagaggg

SEQ ID NO: 494 - DNA, 16 bp, Synthetic construct
  source 1..16: mol_type = other DNA; note = AAV2 wt gDNA for PfAgo
gctcgctcgctcggtg

SEQ ID NO: 495-510 - skipped (16 entries)

SEQ ID NO: 511 - DNA, 15 bp, Synthetic construct
  source 1..15: mol_type = other DNA; note = R1 Forward primer
  modified_base 13..15: mod_base = OTHER; note = phosphorothioate
gaaacccgacaggac

SEQ ID NO: 512 - DNA, 15 bp, Synthetic construct
  source 1..15: mol_type = other DNA; note = R1 Reverse primer
  modified_base 13..15: mod_base = OTHER; note = phosphorothioate
agaatgacttggttg

SEQ ID NO: 513 - DNA, 15 bp, Synthetic construct
  source 1..15: mol_type = other DNA; note = R2 Forward primer
  modified_base 13..15: mod_base = OTHER; note = phosphorothioate
agatccagttcdatg

SEQ ID NO: 514 - DNA, 15 bp, Synthetic construct
  source 1..15: mol_type = other DNA; note = R2 Reverse primer
  modified_base 13..15: mod_base = OTHER; note = phosphorothioate
gtgtcgcccttattc

SEQ ID NO: 515 - DNA, 15 bp, Synthetic construct
  source 1..15: mol_type = other DNA; note = R3 Forward primer
  modified_base 13..15: mod_base = OTHER; note = phosphorothioate
gctacagagttcttg

SEQ ID NO: 516 - DNA, 15 bp, Synthetic construct
  source 1..15: mol_type = other DNA; note = R3 Reverse primer
  modified_base 13..15: mod_base = OTHER; note = phosphorothioate
agaatgacttggttg

SEQ ID NO: 517 - DNA, 15 bp, Synthetic construct
  source 1..15: mol_type = other DNA; note = R4 Forward primer
  modified_base 13..15: mod_base = OTHER; note = phosphorothioate
gaaacccgacaggac

SEQ ID NO: 518 - DNA, 15 bp, Synthetic construct
  source 1..15: mol_type = other DNA; note = R4 Reverse primer
  modified_base 13..15: mod_base = OTHER; note = phosphorothioate
gtgtcgcccttattc

SEQ ID NO: 519 - DNA, 15 bp, Synthetic construct
  source 1..15: mol_type = other DNA; note = R5 Forward primer
  modified_base 13..15: mod_base = OTHER; note = phosphorothioate
gaataagggcgacac

SEQ ID NO: 520 - DNA, 15 bp, Synthetic construct
  source 1..15: mol_type = other DNA; note = R5 Reverse primer
  modified_base 13..15: mod_base = OTHER; note = phosphorothioate
gtcctgtcgggtttc

SEQ ID NO: 521 - DNA, 15 bp, Synthetic construct
  source 1..15: mol_type = other DNA; note = R6 Forward primer
  modified_base 13..15: mod_base = OTHER; note = phosphorothioate
caaccaagtcattct

SEQ ID NO: 522 - DNA, 15 bp, Synthetic construct
  source 1..15: mol_type = other DNA; note = R6 Reverse primer
  modified_base 13..15: mod_base = OTHER; note = phosphorothioate
caagaactctgtagc

SEQ ID NO: 523 - DNA, 15 bp, Synthetic construct
  source 1..15: mol_type = other DNA; note = R7 Forward primer
  modified_base 13..15: mod_base = OTHER; note = phosphorothioate
agatccagttcgatg

SEQ ID NO: 524 - DNA, 15 bp, Synthetic construct
  source 1..15: mol_type = other DNA; note = R7 Reverse primer
  modified_base 13..15: mod_base = OTHER; note = phosphorothioate
agaatgacttggttg

SEQ ID NO: 525 - DNA, 2960 bp, Synthetic construct
  source 1..2960: mol_type = other DNA
agcagaagagctctctgtacactcactcactcactgatccctggataccaaaggtatccagacacccagtctttgactgggtgggatcagtgagtgagtgagtgtacagagagctcttctggagggagtggccaaaaccaactagacaactttgtatactaataatacgactcactatagggaaataagagagaaaagaagagtaagaagaaatataagagccaccatggaagatgccaaaaacattaagaagggcccagcgccattctacccactcgaagacgggaccgccggcgagcagctccacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctgagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtctctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgggatccggagagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtagtgaggatccccacaaagtgacctagagctggcagcaaaaaaaaaaaaaaaaaaaaaaaaaaaaaaaactagcataaccccttggggcctctaaacgggtcttgaggggttttttgaacttcaggaaccccttttaagagtaacataactgctggagtagatgaagatggagggccactttactggctcttcttctctgtacactcactcactcactgatcccacccagtcaaagactgggtgtctggatacctttggtatccagggatcagtgagtgagtgagtgtacagagaagaagagc

SEQ ID NO: 526 - DNA, 3748 bp, Synthetic construct
  source 1..3748: mol_type = other DNA
agaagagcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctcttctggagggagtggccaaaaccaactagacaactttgtatatgaatcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcgccaccatggaagatgccaaaaacattaagaagggcccagcgccattctacccactcgaagacgggaccgccggcgagcagctccacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctgagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtctctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgggatccggagagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtagtgaggatccccacaaagtgacctagagctggcagcaaaataaaatatctttattttcattacatctgtgtgttggttttttgtgtgaacttcaggaaccccttttaagagtaacataactgctggagtagatgaagatggagggccactttactggctcttctaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagaagagc

SEQ ID NO: 527 - DNA, 4687 bp, Synthetic construct
  source 1..4687: mol_type = other DNA
agaagagctctctgtacactcactcactcactgatccctggataccaaaggtatccagacacccagtctttgactgggtgggatcagtgagtgagtgagtgtacagagagctcttctggagggagtggccaaaaccaactagacaactttgtatatgaaggcgcgccgcagggtctccattttgaagcgggaggtttgaacgcgcagccaccatgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcttcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcttacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagagacctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaatttgcttgttaatcaataaaccgtttaattcgtttcagttgaactttggtctcgcggccgcgtttaaacgaggtcctgtattagaggtcacgtgagtgttttgcgacattttgcgacaccatgtggtcacgctgggtatttaagcccgagtgagcacgcagggtctccattttgaagcgggaggtttgaacgcgcattaattaaaacttcaggaaccccttttaagagtaacataactgctggagtagatgaagatggagggccactttactggctcttcttctctgtacactcactcactcactgatcccacccagtcaaagactgggtgtctggatacctttggtatccagggatcagtgagtgagtgagtgtacagaga

SEQ ID NO: 528 - DNA, 9648 bp, Synthetic construct
  source 1..9648: mol_type = other DNA
agaagagctctctgtacactcactcactcactgatccctggataccaaaggtatccagacacccagtctttgactgggtgggatcagtgagtgagtgagtgtacagagagctcttctggagggagtggccaaaaccaactagacaactttgtatatgaatgtacacttaagggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactccagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggcgacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacccgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcttgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagacacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagcttctcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagatgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggagactcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatggatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtgctagcagatctactagtaacttcaggaaccccttttaagagtaacataactgctggagtagatgaagatggagggccactttactggctcttcttctctgtacactcactcactcactgatcccacccagtcaaagactgggtgtctggatacctttggtatccagggatcagtgagtgagtgagtgtacagaga

SEQ ID NO: 529 - DNA, 165 bp, Synthetic construct
  source 1..165: mol_type = other DNA; note = AAV2 5' ITR sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 530 - DNA, 165 bp, Synthetic construct; identical to SEQ ID NO: 529
  source 1..165: mol_type = other DNA; note = AAV2 3' ITR sequence

SEQ ID NO: 531 - DNA, 2955 bp, Synthetic construct
  source 1..2955: mol_type = other DNA
tctctgtacactcactcactcactgatccctggataccaaaggtatccagacacccagtctttgactgggtgggatcagtgagtgagtgagtgtacagagaagaagagccagtggagtggccaaaaccaactagacaggtgagatatctaataatacgactcactataaggaaataagagagaaaagaagagtaagaagaaatataagagccaccatggaagatgccaaaaacattaagaagggcccagcgccattctacccactcgaagacgggaccgccggcgagcagctccacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctgagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtctctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgggatccggagagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtagtgaggatccccacaaagtgacctagagctggcagcaaaaaaaaaaaaaaaaaaaaaaaaaaaaaaaagagaccctagcataaccccttggggcctctaaacgggtcttgaggggttttttgaactgatatcaccccttttaagagtaacataactgctggagtagatgaagatggagggccactttctccagaagagctctctgtacactcactcactcactgatcccacccagtcaaagactgggtgtctggatacctttggtatccagggatcagtgagtgagtgagtgtacagagagctcttct

SEQ ID NO: 532 - DNA, 8560 bp, Synthetic construct
  source 1..8560: mol_type = other DNA
agaagagctctctgtacactcactcactcactgatccctggataccaaaggtatccagacacccagtctttgactgggtgggatcagtgagtgagtgagtgtacagagagctcttctggagttaagggctagatcttagcttacgtcactagagggtccacgtttagtttttaagatccattgatctcctaaacgctgcaagattcgcaacctggtatacttagcgctaggtcctagtgcagcgggactttttttctaaagtcgttgagaggaggagtcgtcagaccagatagctttgatgtcctgatcggaaggatcgttggccccttaagaataccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaacgcgatcgcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggatgagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttggctcccgatcgttgcgttacacacacaattactgctgatcgagtgtagccttcgaatgaagcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtgtcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctagtgaaagttgtctcctcctgcactgactgactgatacaatggatatgccaaaagcaaagcgctatcgcgccttacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataataacatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggagggcctcctggcgccgcaccggcccaaggagcccgcgtggttcctggccaccgtcggcgtgtcgcccgaccaccagggcaagggtctgggcagcgccgtcgtgctccccggagtggaggccgccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctagaactgctagcttgactgactgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtcctggccaacgtgagcaccgtgctgacctccaaatatcgttaagctggagcctgggagccggcctggccctccgccccccccacccccgcagcccacccctggtctttgaataaagtctgagtgagtggccgacagtgcccgtggagttctcgtgacctgaggtgcagggccggcgctagggacacgtccgtgcacgtgccgaggccccctgtgcagctgcaagggacaggcctagccctgcaggcctaactccgcccatcccgcccctaactccgcccagttccgcccattctccgcctcatggctgactaattttttttatttatgcagaggccgaggccgcctcggcctctgagctattccagaagtagtgaggacgcttttttggaggccgaggcttttgcaaagatcgaacaagagacaggacctgcaggttaattaaatttaaatcatgtgagcaaaaggccagcaaaaggccaggaaccgtaaaaaggccgcgttgctggcgttccgaaagaaccggagatattggaacgctcgacgccgctacaagcagtcgtctaagcgccaagctgtctaccaggcggacgcgcagttcggtcgcgtgagaagtaccacaactgtctctatcttaatcgcaagttaacgtgacctactctagtcgacgtagtcagtagtctgcgtgtcacgcgtcaacaagaagcctgacagcacagccggctgtccggcacaaggcacaccagactccgacctgctcgcaacaagtccactcgagttggtcaccaaggtcgcactggctcttcttctctgtacactcactcactcactgatcccacccagtcaaagactgggtgtctggatacctttggtatccagggatcagtgagtgagtgagtgtacagaga

SEQ ID NO: 533 - DNA, 1648 bp, Synthetic construct
  source 1..1648: mol_type = other DNA
agaagagctctctgtacactcactcactcactgatccctggataccaaaggtatccagacacccagtctttgactgggtgggatcagtgagtgagtgagtgtacagagagctcttctggagggagtggccaaaaccaactagacaactttgtatatgaattacgaattcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctcgagctcgtttagtgaaccgtcagatcgcctggagatgccatccacgctgttttgacctccatagaagacaccgggaccgatccagcctcccctcgaagctagtcgattaggcatctcctatggcaggaagaagcggagacagcgacgaagacctcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagctatagcagtagctgaggggacagatagggttatagaagtagtacaagaagcttggcactggccgtcgttttacaacgtcgtgatctgagcctgggagatctctggctaactagggaacccactgcttaagcctcaataaagcttgccttgagtgcttcaagtagtgtgtgcccgtctgttgtgtgactctggtaactagagatcaggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagaacttcaggaaccccttttaagagtaacataactgctggagtagatgaagatggagggccactttactggctcttcttctctgtacactcactcactcactgatcccacccagtcaaagactgggtgtctggatacctttggtatccagggatcagtgagtgagtgagtgtacagaga

SEQ ID NO: 534 - DNA, 7204 bp, Synthetic construct
  source 1..7204: mol_type = other DNA
agaagagctctctgtacactcactcactcactgatccctggataccaaaggtatccagacacccagtctttgactgggtgggatcagtgagtgagtgagtgtacagagagctcttctggagggagtggccaaaaccaactagacaactttgtatatgaatctgcggccgcactagtgctagactgccatgtcgagggattccgggtcactgtgagtgggggaggcagggaagaagggctcacaggacagtcaaaccatgccccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcatcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattccgcggaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctgcggccgctccggaattccatgtgttaatcctcatcctgtctacttgccacacaatcatcacctgccatctgttttccataatccctgatgatctttgcttttcttcttggcactacttttatgtcactattatcttgtattactactgccccttcacctttccagaggagctttgctggtcctttccaaactggatctctgctgtccctgtaataaacccgaaaattttgaatttttgtaatttgtttttgtaattctttagtttgtatgtctgttgctattatgtctactattctttcccctgcactgtaccccccaatccccccttttcttttaaaattgtggatgaatactgccatttgtactgctgtcttaagatgttcagcctgatctcttacctgtcctataattttctttaattctttattcatagattctattactccttgactttggggattgtagggaatgccaaattcctgcttgatccccgcccaccaacaggcggccttaactgtagtactggtgaaattgctgccattgtctgtatgtactgtttttactggccatcttcctgctaattttaagaggaagtatgctgtttcttgccctgtctctgctggaattacttctgcttctatatatccactggctacatgaactgctaccaagataacttttccttctaaatgtgtacaatctagctgccatattcctgggctacagtctacttgtccatgcatggcttccccttttagctgacatttatcacagctggctactatttcttttgctactacaggtggtaggttaaaatcactagccattgctctccaattactgtgatatttctcatgttcttcttgggccttatctattccatctaaaaatagtactttcctgattccagcactgaccaatttatctacttgttcatttcctccaattcctttgtgtgctggcacccatgccaggtagactttttccttttttattaactgctctattatttgactgactaactctgattcactcttatctggttgtgcttgaatgattcccaatgcatattgtgagtctgtcactatgtttacttctaatcccgaatcctgcaaagctagatgaattgcttgtaactcagtcttctgatttgttgtgtccgttagggggacaactttttgtcttcctctgtcagttacatatcctgcttttcctaatttagtttccctattggctgccccatctacatagaaagtttctgctcctattatgggttctttctctaactgataccataacttcactaagggaggggtattgacaaactcccactcaggaatccaggtggcttgccaatactctgtccaccatgcttcccatgtttccttttgtatgggtaatttaaatttaggagtctttccccatattactatgctttctgtggctattttttgtactgcctctgttaattgtttcacatcattagtgtgggcacccttcattcttgcatactttcctgttttcagatttttaaatggctcttgataaatttgatatgtccattggccttgcccctgcttctgtatttctgctattaagtcttttgatgggtcataatacactccatgtaccggttcttttagaatctccctgttttctgccagttctagctctgcttcttctgttagtggtactacttctgttagtgctttggttcccctaagaagtttacataattgccttactttaatccctgcataaatctgacttgcccaattcaattttcccactaatttctgtatgtcattgacagtccagctgtccttttctggcagcactataggctgtactgtccatttatcaggatggagttcataacccatccaaaggaatggaggttctttctgatgttttttgtctggtgtggtaaatccccacctcaacagatgttgtctcagttcctctatttttgttctatgctgccctatttctaagtcagatcctacatacaaatcatccatgtattgatagatgactatgtctggattttgttttctaaaaggctctaagatttttgtcatgctacactggaatattgctggtgatcctttccatccctgtggaagcacattgtactgatatctaatccctggtgtctcattgtttatactaggtatggtaaatgcagtatacttcctgaagtctttatctaagggaactgaaaaatatgcatcgcccacatccagtactgttactgattttttctgttttaaccctgcaggatgtggtattcctaattgaacttcccagaaatcttgagttctcttattaagttctctgaaatctactaattttctccatttagtactgtcttttttctttatggcaaatactggagtattgtatggattttcaggcccaatttttgaaatttttccttccttttccatttctgtacaaatttctactaatgcttttattttttcttctgtcaatggccattgtttaacttttgggccatccattcctggctttaattttactggtacagtctcaataggactaatgggaaaatttaaagtgcagccaatctgagtcaacagatttcttccaattatgttgacaggtgtaggtcctactaatactgtacctatagctttatgtccgcagatttctatgagtatctgatcatactgtcttactttgataaaacctccaattccccctatcatttttggtttccatcttcctggcaaattcatttcttctaatactgtatcatctgctcctgtatctaatagagcttcctttaattgcccccctatctttattgtgacgaggggtcgctgccaaagagtgatctgagggaagctaaaggatacagttccttgtctatcggctcctgcttctgagagggagttgttgtctcttccccaaacctgaagctctcttctggtggggctgttggctctggtctgctctgaagaaaattccctggccttcccttgtgggaaggccagatcttccctaaaaaattagcctgtctctcagtacaatctttcatttggtgtccttcctttccacatttccaacagccctttttcctaggggccctgcaatttttggctatgtgcccttctttgccacaattgaaacacttaacagtctttctttggttcctaaaattgcctttctgtatcattatggtagctggatttgttacttggctcattgcttcagccaaaactcttgctttatggccgggtccccccactccctgacatgctgtcatcatttcttctagtgtcgctcctggtcccaatgcttttaaaatagtcttacaatctgggttcgcattttggaccaacaaggtttctgtcatccaattttttacctcttgtgaagcttgctcggctcttagagttttatagaatcggtctacatagtctctaaagggttcctttggtccttgtcttatgtccagaatgctggtagggctatacattcttactattttatttaatcccaggattatccatcttttatagatttctcctactgggataggtggattatgtgtcatccatcctatttgttcctgaagggtactagtagttcctgctatgtcacttccccttggttctctcatctggcctggtgcaataggccctgcatgcactggatgcactctatcccattctgcagcttcctcattgatggtctcttttaacatttgcatggctgcttgatgtccccccactgtgtttagcatggtgtttaaatcttgtggggtggctccttctgataatgctgaaaacatgggtatcacttctgggctgaaagccttctcttctactacttttacccatgcatttaaagttctaggtgatatggcctgatgtaccatttgcccctggatgttctgcactatagggtaattttggctgacctgattgctgtgtcctgtgtcagctgctgcttgctgtgcttttttcttacttttgttttgctcctcctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccagcgatctaattctcccccgcttaatactgacgctctcgcacccatggcggcggcagatctcgaattcagatctcacgtgctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccgagctcgggaccacatgtaagcttcgaggggaggctggatcggtcccggtgtcttctatggaggtcaaaacagcgtggatggcatctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtaatgttggacatgagccaatataaatgtacatattatgatatggatacaacgtatgcaataacttcaggaaccccttttaagagtaacataactgctggagtagatgaagatggagggccactttactggctcttcttctctgtacactcactcactcactgatcccacccagtcaaagactgggtgtctggatacctttggtatccagggatcagtgagtgagtgagtgtacagaga

SEQ ID NO: 535 - DNA, 4005 bp, Synthetic construct
  source 1..4005: mol_type = other DNA
agaagagctctctgtacactcactcactcactgatccctggataccaaaggtatccagacacccagtctttgactgggtgggatcagtgagtgagtgagtgtacagagagctcttctggagggagtggccaaaaccaactagacaactttgtatatgaagcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagatgccatccacgctgttttgacctccatagaagacaccgggaccgatccagcctccggtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattcctcgacggatccctcgaggaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggcaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattcctcgagggatccgtcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacggatcggccgcaattcgtaatcataacttcaggaaccccttttaagagtaacataactgctggagtagatgaagatggagggccactttactggctcttcttctctgtacactcactcactcactgatcccacccagtcaaagactgggtgtctggatacctttggtatccagggatcagtgagtgagtgagtgtacagaga